(19) United States
(12) Patent Application Publication
(10) Pub. No.: US 2024/0110199 A1
(43) Pub. Date: Apr. 4, 2024

(54) NOVEL GENETIC LOCI ASSOCIATED WITH DISEASE RESISTANCE IN SOYBEANS

(71) Applicant: Syngenta Crop Protection AG, Basel (CH)

(72) Inventors: Qingli Liu, Research Triangle Park, NC (US); Thomas Joseph Curley, Jr., Research Triangle Park, NC (US); Becky Welsh Breitinger, Research Triangle Park, NC (US); Andrew David Farmer, Santa Fe, NM (US); Xiaoping Tan, Research Triangle Park, NC (US); Yinping Lucy Qin, Research Triangle Park, NC (US)

(73) Assignee: Syngenta Crop Protection AG, Basel (CH)

(21) Appl. No.: 18/264,857

(22) PCT Filed: Feb. 4, 2022
(86) PCT No.: PCT/US22/15172

§ 371 Date: Aug. 9, 2023

Related U.S. Application Data
(60) Provisional application No. 63/147,849, filed on Feb. 10, 2021.
(60) Provisional application No. 63/209,005, filed on Jun. 10, 2021.

(51) Int. Cl. C12N 15/82; A01H 6/54; C07K 14/415

(52) U.S. Cl. CPC C12N 15/8282; A01H 6/542; C07K 14/415

(57) ABSTRACT

The present invention relates to methods and compositions for identifying, selecting and/or producing a pathogen resistant plant or germplasm (e.g., soybean plant or germplasm) using genes derived from Glycine tomentella. Candidate genes capable of conferring pathogen resistance (e.g., to Asian Soy Rust) are provided. A plant or germplasm that has been identified, selected and/or produced by any of the methods of the present invention is also provided. Pathogen resistant seeds, plants and germplasms are also provided.

### RELATED APPLICATIONS

This application claims priority to US Provisional Patent Application Nos. 63/147,849 filed 10 Feb. 2021 and 63/209,005 filed 10 Jun. 2021, the contents of which are incorporated by reference herein in their entirety.

### FIELD OF THE INVENTION

The present invention relates to compositions and methods for identifying, selecting and producing enhanced disease and/or pathogen resistant plants using novel resistance genes.

### STATEMENT REGARDING ELECTRONIC SUBMISSION OF A SEQUENCE LISTING

A Sequence Listing in ASCII text format, submitted under 37 C.F.R. § 1.821, entitled “82250PCT_ST25.txt”, approximately ˜9.07 MB in size, generated on Jan. 7, 2022 and filed via EFS-Web is provided in lieu of a paper copy. This Sequence Listing is hereby incorporated by reference into the specification in its entirety.

### BACKGROUND

Plant pathogens are known to cause considerable damage to important crops, resulting in significant agricultural losses with widespread consequences for both the food supply and other industries that rely on plant materials. As such, applicant desires to reduce the incidence and/or impact of agricultural pathogens on crop production.

Several pathogens have been associated with damage to soybeans, which individually and collectively have the potential to cause significant yield losses in the United States and throughout the world. Exemplary pathogens include, but are not limited to fungi (e.g., genus Phytophthora and Asian Soybean rust Phakopsora pachyrhizi), nematodes (e.g., genus Meloidogyne, particularly, Meloidogyne javanica), and soybean stem canker. Given the significant threat to global food supplies that these pathogens present as well as the time and expense associated with treating soybean crops to prevent yield loss, new methods for producing pathogen resistant soybean cultivars are needed. What is needed is novel resistance genes (herein, “R-Genes”) that can be introduced into commercial soybean plants to control soybean pathogens.

### SUMMARY OF THE INVENTION

This summary lists several embodiments of the presently disclosed subject matter, and in many cases lists variations of these embodiments. Thus, in embodiments, it is an object of the presently disclosed subject matter to provide methods for conveying pathogen resistance into non-resistant plants and germplasm. Further, the presently disclosed subject matter provides novel Glycine max lines comprising in its genome a chromosome interval, loci, and/or gene that is derived from Glycine tomentella and confers pathogen resistance in said novel Glycine max line, which object is achieved in whole or in part by the presently disclosed subject matter.

In embodiments of the present invention, the plant or germplasm is a soybean plant or germplasm, and the pathogen is a soybean pathogen, particularly a soybean fungal pathogen such as Asian Soybean Rust (e.g., Phakopsora pachyrhizi; herein “ASR”).

The present invention provides for chromosomal intervals derived from Glycine tomentella, particularly accession line PI505267, that when introduced into a plant (e.g., a soybean plant such as Glycine max strain Williams 82 or an elite Glycine max line) are sufficient to confer increased rust resistance, such as increased Asian soybean rust (“ASR”) resistance, as compared to a control plant not comprising said chromosomal interval.

The invention also provides for novel proteins and nucleic acids derived from the chromosomal interval of Glycine tomentella accession line PI505267 that confer rust resistance. In embodiments of the present invention, the novel protein is the protein of SEQ ID NO: 5 or functional variants thereof, such as variants that are substantially similar (e.g., having at least 85%, at least 90% or at least 95% sequence identity) and that confer increased ASR resistance on a plant in which the protein is expressed. In further embodiments, nucleic acids comprising and/or encoding novel R-genes derived from the chromosomal interval of Glycine tomentella accession line PI505267 are provided that, when expressed in a plant, confer rust resistance. In embodiments, the nucleic acid comprises a nucleotide sequence encoding the novel protein of SEQ ID NO: 5, or functional variants thereof. In other embodiments, the nucleic acid comprises the nucleotide sequence of any of SEQ ID NOS: 2-4 and 11-12; or a sequence that is substantially similar and capable of conferring ASR resistance (such as sequences that have at least 85%, at least 90% or at least 95% sequence identity to any one of SEQ ID NOs: 2-4 and 11-12).

The present invention also provides for expression cassettes, vectors, and DNA constructs comprising the novel R-gene and/or encoding the novel protein of the invention. In embodiments, the expression cassette allows for transgenic expression of the novel nucleic acid and/or protein via a promoter operably connected thereto. In other embodiments, the DNA constructs allow for gene editing of the novel nucleic acid and/or protein.

The present invention also encompasses novel plants that have stably incorporated into their genome a novel nucleic acid sequence derived from the chromosomal interval of Glycine tomentella accession line PI505267 (such as a nucleic acid encoding the novel protein of SEQ ID NO: 5, or a substantially similar polypeptide) that confers the novel plant with increased pathogen resistance as compared to control plants not comprising the nucleic acid. In embodiments, the plants are novel Glycine max plants and/or novel elite Glycine max plants that have increased ASR resistance as compared to the control plants. In embodiments, the novel nucleic acid sequence is introduced into the plant through transgenic expression, through introgression, through known breeding methods, or through gene editing.

The present invention also provides for methods of producing plants having increased ASR resistance through the introduction of a nucleic acid sequence encoding the novel R-gene and/or protein of the invention. In particular embodiments, a method of producing a transgenic plant with improved resistance against ASR is provided by introducing a nucleic acid molecule comprising a nucleotide sequence encoding the novel protein (e.g., encoding the protein of SEQ ID NO: 5 or a substantially similar sequence, or comprising the nucleotide sequence of any of SEQ ID NOS: 2-4, 11-12, or a substantially similar sequence). In embodiments, the nucleic acid is introduced through an expression cassette comprising the nucleic acid sequence, the expression cassette introduced to a recipient plant to obtain a transgenic plant, wherein the transgenic plant has increased resistance against ASR compared to the recipient plant.

Compositions and methods for identifying, selecting and producing Glycine plants (including wild Glycines, elite Glycine lines and Glycine max lines) with enhanced pathogen (e.g., rust) resistance are provided. Pathogen resistant plants and germplasm (e.g., soybean plants and germplasms) are also provided. In some embodiments, methods of producing an ASR resistant soybean plant are provided.

In some embodiments, methods of identifying a rust resistant soybean plant or germplasm are provided. Such methods may comprise detecting, in the soybean plant or germplasm, a genetic loci or molecular marker (e.g. SNP or a Quantitative Trait Loci (QTL)) associated with enhanced disease resistance, in particular ASR resistance. In some embodiments the genetic loci or molecular marker associates with the presence of a chromosomal interval comprising the nucleotide sequence of SEQ ID NO 1, 2-4, 11, or 12, or a portion thereof, wherein the portion thereof associates with ASR resistance.

The foregoing and other objects and aspects of the present invention are explained in detail in the drawings and specification set forth below.

### BRIEF DESCRIPTION OF THE DRAWINGS

FIG. 1 is an illustration of binary vector 25845.

FIG. 2 is an illustration of binary vector 25899.

FIG. 3 shows photographs of rust bioassay experiments conducted on leaves collected from primary soybean events generated from constructs 25845 and 25899. The primary soybean events from both constructs (GVG01375963 of construct 25845 and GVG013773804 of construct 25899) show reddish brown lesions against all three rust populations tested (RTP1, BRS and SUL) while leaves from the control (which has the same genetic background without the transgene) show a tan reaction and heavy sporulation.

FIG. 4 is a table of disease resistance ratings of primary soybean events generated from construct 25845 and 25899 relative to the control. TO events, GVG01375963, generated from construct 25845, and GVG01373804, generated from 25899, repeatedly show high levels of disease resistance compared to the control (which comprises the same genetic background without the transgene). The results use standard soy rust rating scales with Reddish-brown (RB) types indicative of being resistant while Tan ratings are indicative of being susceptible. Numbers after the RB ratings are based on a combination of density of lesions or size of the lesions with a 1-4 scale from high to intermediate resistance, and indication of no sporulation (NSP) or very little sporulation (SPL). Numbers after Tan ratings are based on a combination of density of pustules and level of sporulation with 1-5 scale from low to high sporulation.

FIG. 5 is a graph showing relative expression (y-axis) of soybean rust β-tubulin gene on two events constructed from binary vector 25845 and 25899 at 14 days post inoculation with 3 rusts populations (SUL, BRS and RTP1). Levels of resistance were measured molecularly with fungal β-tubulin via qRT-PCR on the events GVG01375963 and GVG013773804 and compared to measurements of the control. The quantitative measurement is consistent with the phenotypic observations that the events showed high levels of resistance.

FIG. 6 is an illustration of binary vector 25992.

FIG. 7 is an illustration of binary vector 26015.

FIG. 8 is an illustration of binary vector 25950.

FIG. 9 shows photographs of rust bioassay experiments conducted on leaves collected from primary soybean events generated from construct 25950. Both the primary soybean events show reddish brown lesions against tested rust populations tested (RTP1, BRS and SUL) while leaves from the control (which has the same genetic background without the transgene) show a tan reaction and heavy sporulation. This indicates the strong resistance of the events to rust populations.

FIG. 10 is a graph showing relative expression (y-axis) of soybean rust β-tubulin gene on both the events constructed from binary vector 25950 at 14 days post inoculation with 3 rusts populations (SUL, BRS and RTP1). Levels of resistance were measured molecularly with fungal 3-tubulin via qRT-PCR on the events and compared to measurements of the control. Leaves from the TO events showed levels that were <1% of the susceptible controls. The quantitative measurement is consistent with the phenotypic observations that the events showed high levels of resistance.

FIG. 11 is a diagram depicting an example method of introgressing a genomic region associated with ASR resistance from G. tomentella into wild Glycine by doubling a G. max line that is ASR susceptible to create a tetraploid G. max line, and then crossing the tetraploid G. max line that is ASR susceptible with a diploid G. tomentella line that is ASR resistant. In some embodiments, the G. max line is an elite G. max line. In some embodiments, the introgressed genomic region is SEQ ID NO: 1 or a functional fragment thereof that confers increased ASR resistance. In some embodiments, the introgressed genomic region is any one of SEQ ID NOS: 2-4, 11-12.

### BRIEF DESCRIPTION OF THE SEQUENCE LISTING

SEQ ID NO: 1 is a chromosomal interval derived from Glycine tomentella line accession number PI505267, herein also referred to as “Contig 0133”. Contig 0133 has been mapped to G. tomentella (genotype D3) at an approximate interval of ˜9.28 MB-16.48 MB (that is was ˜33.8 Mbp in size) on chromosome 3. Genetic population mapping studies for PI505267 indicate that Glycine tomentella Chromosome 3 contains chromosomal intervals highly associated with ASR resistance (e.g., as corresponding to SEQ ID NO: 1). This chromosomal interval or portions thereof may be introduced (e.g., transgenically, through gene editing, and/or introgressed through use of embryo rescue & marker assisted breeding (MAB)) into Glycine max lines to create Glycine max lines resistant to various diseases such as ASR. In embodiments, “Contig 0133” is on Chromosome 3 in the span from position 3004342-36810588 of the reference genome.

Further research into the interval led to the discovery of a plurality of putative causative genes potentially associated with increased pathogen resistance trait (herein also referred to as R-genes). The putative genes from the above interval are located on or corresponding to Glycine tomentella Chromosome 3. Each of the causative genes were identified and isolated, their functions validated and their efficacy in resisting soybean pathogens were assessed. As elaborated in the Examples below, the chromosomal interval of SEQ ID NO: 1, derived from Glycine tomentella accession PI505267, can be used as a source for the R-genes corresponding to SEQ ID NOs: 2-5 and 7-8.

SEQ ID NO: 2 is a genomic DNA sequence of a soy rust resistance candidate gene (herein referred to as “GtoRG30”) from PI505267 encoding a protein containing Toll/Interleukin-1 receptor (TIR), nucleotide-binding site (NBS), and leucine rich-repeat (LRR) domains (herein, a “TNL” R-gene motif). The gene is syntenic to Glyma.05G165800 (Soy_william82_v2). The genomic DNA fragment has been mapped to an approximate interval of ˜11.44 MB-11.46 MB on chromosome 3 of G. tomentella. The genomic DNA sequence includes the gene with its native 5′UTR and 3′UTR and native introns.

SEQ ID NO: 3 is a genomic DNA sequence for soy rust resistance candidate gene GtoRG30 with its native 5′UTR and 3′UTR and with the first native intron replaced with Arabidopsis intron, iAtBAF60-01 (SEQ ID NO: 21).

SEQ ID NO: 4 is a DNA coding sequence for soy rust resistance candidate gene GtoRG30 that encodes a protein containing toll/interleukin receptor-1 (TIR), nucleotide-binding site (NBS), and leucine rich repeat (LRR) domains. The first native intron was replaced with Arabidopsis intron, iAtBAF60-01 (SEQ ID NO: 21).

SEQ ID NO: 5 is the amino acid sequence of the protein encoded by soy rust resistance candidate gene GtoRG30. The protein of SEQ ID NO: 5 is encoded by any of the nucleic acid sequences of SEQ ID NOS: 2-4 and 11-12.

SEQ ID NO: 6 is a genomic DNA sequence for a soy rust resistance candidate gene from PI505267 that encodes a protein comprising coiled-coiled (CC), nucleotide-binding site (NBS), and leucine rich repeat (LRR) domains (herein, a “CNL” R-gene motif).

SEQ ID NO: 7 is a genomic DNA sequence for another soy rust resistance candidate gene from PI505267 that encodes a protein comprising coiled-coiled (CC), nucleotide-binding site (NBS), and leucine rich repeat (LRR) domains. The genes of SEQ ID NOS. 6-7 are syntenic to Glyma.05G165600 (Soy_william82_v2).

SEQ ID NOS: 8-9 is the DNA sequence for a primer pair that can be used for generating an amplicon (such as via PCR) comprising the soy rust resistance candidate gene GtoRG30.

SEQ ID NO: 10 is the DNA sequence for a probe that can be used for detecting a polynucleotide, such as an amplicon, comprising soy rust resistance gene GtoRG30.

SEQ ID NO: 11 is a genomic DNA sequence (gGtoRG30-01) for soy rust resistance candidate gene GtoRG30 with its native promoter (SEQ ID NO: 15) and terminator (SEQ ID NO: 18) and with the first native intron replaced with Arabidopsis intron, iAtBAF60-01 (SEQ ID NO: 21).

SEQ ID NO: 12 is the cDNA sequence for soy rust resistance candidate gene GtoRG30.

SEQ ID NO: 13 is the DNA sequence of a promoter from the Medicago truncatula gene Mt12344. The promoter is active in plant cells and can be used to drive the expression of a heterologous nucleic acid sequence, such as any R-gene.

SEQ ID NO: 14 is the DNA sequence of a promoter from the Medicago truncatula gene Mt51186. The promoter is active in plant cells and can be used to drive the expression of a heterologous nucleic acid sequence, such as any R-gene.

SEQ ID NO: 15 is the DNA sequence of an endogenous promoter (“RG30_promoter”) from the soy rust resistance candidate gene herein referred to as “GtoRG30”. It consists of the 5′-non-transcribed sequence and the 5′ UTR (SEQ ID NO: 19) of the candidate gene.

SEQ ID NO: 16 is the DNA sequence of a terminator from the Medicago truncatula gene Mt12344.

SEQ ID NO: 17 is the DNA sequence of a terminator from the Medicago truncatula gene Mt51186. The promoter is active in plant cells and can be used to drive the expression of a heterologous nucleic acid sequence, such as any R-gene.

SEQ ID NO: 18 is the DNA sequence of an endogenous terminator (“RG30_terminator”) from the soy rust resistance candidate gene herein referred to as “GtoRG30”. It consists of the 3′-non-transcribed sequence and the 3′ UTR (SEQ ID NO: 20) of the candidate gene.

SEQ ID NO: 19 is the DNA sequence of the 5′ UTR from the soy rust resistance candidate gene herein referred to as “GtoRG30”.

SEQ ID NO: 20 is the DNA sequence of the 3′ UTR from the soy rust resistance candidate gene herein referred to as “GtoRG30”.

SEQ ID NO: 21 is the DNA sequence of intron iAtBAF60-01 of Arabidopsis thaliana BAF60 homolog.

ASR resistance can be introduced into G. max plants using a nucleic acid having a sequence encoding a polypeptide that is substantially identical to SEQ ID NO: 5 (e.g., having at least 70% sequence identity to SEQ ID NO: 5). For example, ASR resistance can be introduced through the introduction of a nucleic acid comprising any one of SEQ ID NOs: 2-4, 6-7, 11-12 or a nucleic acid sequence substantially identical to any one of SEQ ID NOS: 2-4, 6-7, and 11-12 (e.g., having at least 70% sequence identity).

SEQ ID NOS: 22-237, listed at Table 6, describe the DNA sequence of example assay components, including primers and probes, that can be used to detect and differentiate between favorable and unfavorable alleles associated with a given SNP position within the chromosomal interval of SEQ ID NO: 1.

### DETAILED DESCRIPTION OF THE INVENTION

The presently disclosed subject matter relates to compositions and methods for introducing novel resistance genes (herein “R-Genes”) encoding novel proteins for pathogen resistance into commercial plants to control plant pathogens. The methods involve transforming organisms with nucleic acid molecules having nucleotide sequences encoding the novel proteins for pathogen resistance of the invention. The nucleotide sequences of the invention are useful for generating plants, particularly soybean plants, that show increased resistance to plant pathogens, particularly fungal pathogens such as Asian Soybean Rust (herein, “ASR”). Thus, transformed plants, plant cells, plant tissues and seeds are provided. Compositions include nucleic acids and proteins relating to pathogen resistant plants as well as transformed plants, plant tissues and seeds. Nucleotide sequences of nucleic acids comprising the novel R-genes and/or encoding the amino acid sequence of the novel resistance proteins are disclosed. The sequences find use in the construction of vectors and expression cassettes for subsequent transformation into plants of interest, as probes and/or primers for the detection and isolation of the R-genes, and the like. In particular embodiments, the compositions and methods are used to introduce novel R-genes into soybean plants to control soybean pathogens, such as fungal pathogens (e.g., ASR) and/or nematodes.

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

### Definitions

Unless defined otherwise, all technical and scientific terms used herein have the same meaning as commonly understood to one of ordinary skill in the art to which the presently disclosed subject matter belongs.

Although the following terms are believed to be well understood by one of ordinary skill in the art, the following definitions are set forth to facilitate understanding of the presently disclosed subject matter.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount. As used herein, phrases such as “between about X and Y” mean “between about X and about Y” and phrases such as “from about X to Y” mean “from about X to about Y.”

As used herein, phrases such as “between about X and Y”, “between about X and about Y”, “from X to Y” and “from about X to about Y” (and similar phrases) should be interpreted to include X and Y, unless the context indicates otherwise.

As used herein, a “coding sequence” or “CDS” is a nucleic acid sequence that is transcribed into RNA such as mRNA, rRNA, tRNA, snRNA, sense RNA or antisense RNA. In embodiments, the RNA is then translated to produce a protein. In example embodiments, the CDS is derived from a cDNA sequence and includes the sequence of spliced exons of a transcript in DNA notation and does not include any intron or 5′ or 3′-untranslated regions (UTRs). In other example embodiments, the CDS is derived from a genomic DNA sequence and includes the sequence of spliced exons of a transcript in DNA notation as well as one or more introns, and 5′ and/or 3′-untranslated regions (UTRs).

As used herein, a “codon optimized” nucleotide sequence means a nucleotide sequence of a recombinant, transgenic, or synthetic polynucleotide wherein the codons are chosen to reflect the particular codon bias that a host cell or organism may have. This is typically done in such a way so as to preserve the amino acid sequence of the polypeptide encoded by the codon optimized nucleotide sequence. In certain embodiments, a nucleotide sequence is codon optimized for the cell (e.g., an animal, plant, fungal or bacterial cell) in which the construct is to be expressed. For example, a construct to be expressed in a plant cell can have all or parts of its sequence codon optimized for expression in a plant. See, for example, U.S. Pat. No. 6,121,014. In embodiments, the polynucleotides of the invention are codon-optimized for expression in a plant cell (e.g., a dicot cell or a monocot cell) or bacterial cell.

The term “comprise”, “comprises” or “comprising,” when used in this specification, indicates the presence of the stated features, integers, steps, operations, elements, or components, but does not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim “and those that do not materially alter the basic and novel characteristic(s)” of the claimed invention. Thus, the term “consisting essentially of” when used in a claim of this invention is not intended to be interpreted to be equivalent to “comprising.”

The term “corresponding to” in the context of nucleic acid sequences or protein sequences means that when the nucleic acid sequences or amino acid sequences of certain sequences are aligned with each other, the nucleic acids or amino acids that “correspond to” certain enumerated positions in the present invention are those that align with these positions in a reference sequence, but that are not necessarily in these exact numerical positions relative to a particular nucleic acid sequence of the invention. Optimal alignment of sequences for comparison can be conducted by computerized implementations of known algorithms or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

Unless otherwise stated, identity and similarity will be calculated by the Needleman-Wunsch global alignment and scoring algorithms (Needleman and Wunsch (1970) J. Mol. Biol. 48(3):443-453) as implemented by the “needle” program, distributed as part of the EMBOSS software package (Rice, P. Longden, and Bleasby, A., EMBOSS: The European Molecular Biology Open Software Suite, 2000, Trends in Genetics 16, (6) pp 276-277, versions 6.3.1 available from EMBnet at embnet.org/resource/emboss and emboss.sourceforge.net, among other sources) using default gap penalties and scoring matrices (EBLOSUM62 for protein and EDNAFULL for DNA). Equivalent programs may also be used. By “equivalent program” is intended any sequence comparison program that, for any two sequences in question, generates an alignment having identical nucleotide residue matches and an identical percent sequence identity when compared to the corresponding alignment generated by needle from EMBOSS version 6.3.1.

Additional mathematical algorithms are known in the art and can be utilized for the comparison of two sequences. See, for example, the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLAST programs of Altschul et al. (1990) J. Mol. Biol. 215:403. BLAST nucleotide searches can be performed with the BLASTN program (nucleotide query searched against nucleotide sequences) to obtain nucleotide sequences homologous to nucleic acid molecules of the invention, or with the BLASTX program (translated nucleotide query searched against protein sequences) to obtain protein sequences homologous to nucleic acid molecules of the invention. BLAST protein searches can be performed with the BLASTP program (protein query searched against protein sequences) to obtain amino acid sequences homologous to protein molecules of the invention, or with the TBLASTN program (protein query searched against translated nucleotide sequences) to obtain nucleotide sequences homologous to protein molecules of the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

“Expression cassette” as used herein means a nucleic acid molecule capable of directing expression of at least one polynucleotide of interest, such as a nucleic acid comprising the sequence of an R-gene polynucleotide that encodes a protein of the invention, the protein conferring increased pathogen resistance when expressed in an appropriate host cell, the expression cassette comprising a promoter operably linked to the polynucleotide of interest which is operably linked to a termination signal. An “expression cassette” also typically comprises additional polynucleotides to facilitate proper translation of the polynucleotide of interest. The expression cassette may also comprise other polynucleotides not related to the expression of a polynucleotide of interest but which are present due to convenient restriction sites for removal of the cassette from an expression vector. In embodiments, at least one of the components in the expression cassette may be heterologous (i.e., foreign) with respect to at least one of the other components (e.g., a heterologous promoter operatively associated with a polynucleotide of interest). The expression cassette may also be one that is naturally occurring but has been obtained in a recombinant form useful for heterologous expression. Typically, however, the expression cassette is heterologous with respect to the host, i.e., the expression cassette (or even the polynucleotide of interest) does not occur naturally in the host cell and has been introduced into the host cell or an ancestor cell thereof by a transformation process or a breeding process. The expression of the polynucleotide(s) of interest in the expression cassette is generally under the control of a promoter. The promoter may be a heterologous promoter or an endogenous (or native) promoter derived from the same source as the nucleic acid of interest. In the case of a multicellular organism, such as a plant, the promoter can also be specific or preferential to a particular tissue, or organ, or stage of development (as described in more detail herein). An expression cassette, or fragment thereof, can also be referred to as “inserted polynucleotide” or “insertion polynucleotide” when transformed into a plant.

The term “introduced” as used herein, in connection to a plant, means accomplished by any manner including but not limited to; introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs).

As used herein, the term “wild glycine” refers to a perennial Glycine plant, for example any one of G. canescens, G. argyrea, G. clandestine, G. latrobeana, G. albicans, G. aphyonota, G. arenaria, G. curvata, G. cyrtoloba, G. dolichocarpa, G. falcate, G. gracei, G. hirticaulis, G. lactovirens, G. latifolia, G. microphylla, G. montis-douglas, G. peratosa, G. pescadrensis, G. pindanica, G. pullenii, G. rubiginosa, G. stenophita, G. syndetika, or G. tomentella.

As used herein, the term “allele” refers to one of two or more different nucleotides or nucleotide sequences that occur at a specific locus.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” refers to a marker whose presence or absence can be used to predict whether and/or to what extent a plant will display a pathogen resistant phenotype. In example embodiments of the present invention, a nucleic acid (e.g., a chromosomal interval) comprising the R-gene of interest and capable of conferring enhanced pathogen resistance may be detected, identified or selected based on the presence of a “favorable” marker, such as any of the favorable markers of Table 1 and/or 2.

A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9: 275 (1993)), an amplified fragment length polymorphism (AFLP) (Vos et al., Nucleic Acids Res. 23: 4407 (1995)), a single nucleotide polymorphism (SNP) (Brookes, Gene 234: 177 (1993)), a sequence-characterized amplified region (SCAR) (Paran and Michelmore, Theor. Appl. Genet. 85: 985 (1993)), a sequence-tagged site (STS) (Onozaki et al., Euphytica 138: 255 (2004)), a single-stranded conformation polymorphism (SSCP) (Orita et al., Proc. Natl. Acad. Sci. USA 86: 2766 (1989)), an inter-simple sequence repeat (ISSR) (Blair et al., Theor. Appl. Genet. 98: 780 (1999)), an inter-retrotransposon amplified polymorphism (IRAP), a retrotransposon-microsatellite amplified polymorphism (REMAP) (Kalendar et al., Theor. Appl. Genet. 98: 704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art (e.g., using PCR). A large number of soybean molecular markers are known in the art, and are published or available from various sources, such as the SoyBase internet resource.

Markers corresponding to genetic polymorphisms between members of a population can be detected by methods well-established in the art. These include, e.g., nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), and/or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele” also described as an “allele of a marker locus” can refer to one of a plurality of polymorphic nucleotide sequences found at a marker locus in a population that is polymorphic for the marker locus.

“Marker-assisted selection” (MAS) is a process by which phenotypes are selected based on marker genotypes. In some embodiments, marker genotypes are used to identify plants that will be selected for a breeding program or for planting. In some embodiments, marker genotypes are used to identify plants that will not be selected for a breeding program or for planting (i.e., counter-selected plants), allowing them to be removed from the breeding/planting population.

As used herein, the terms “marker locus” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the terms “marker probe” and “probe” refer to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides may be used for nucleic acid hybridization. Alternatively, in some aspects, a marker probe refers to a probe of any type that is able to distinguish (i.e., genotype) the particular allele that is present at a marker locus.

As used herein, the terms “molecular marker” or “genetic marker” may be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein are also referred to as hybridization markers when located on an indel region. Thus, the marker need only indicate whether the indel region is present or absent. Any suitable marker detection technology may be used to identify such a hybridization marker, e.g., SNP technology is used in the examples provided herein.

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval defined by and including,” used in reference to particular loci and/or alleles, refers to a chromosomal interval delimited by and encompassing the stated loci/alleles.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “desired allele”, “favorable allele” and “allele of interest” are used interchangeably to refer to an allele associated with a desired trait (e.g. ASR resistance). In example embodiments, the desired allele may be detected or identified via marker based assays, such as using a SNP marker assay.

As used herein, the terms “enhanced pathogen resistance”, “enhanced disease resistance”, and “conferring or enhancing resistance to a pathogen” refers to an improvement, enhancement, or increase in a plant's ability to endure and/or thrive despite being infected with a pathogen or disease (e.g. Asian soybean rust) as compared to one or more control plants (e.g., one or both of the parents, or a plant lacking a nucleic acid comprising an R-gene or marker associated with enhanced pathogen resistance to respective pathogen/disease). An enhanced plant pathogen resistance comprises any statistically significant increase in resistance to the plant pathogen, including, for example, an increase of at least 1%, 5%, 10%, 20%, 30%, 40%, 50%, 60%, 70%, 80%, 90%, 100% or higher. The control plants may be fully susceptible to the pathogen or have limited resistance to the pathogen. Enhanced disease resistance includes any mechanism (other than whole-plant immunity or resistance) that reduces the expression of symptoms indicative of infection for a respective disease such as Asian soybean rust, soybean cyst nematode, Phytophthora, etc. Conferring or enhancing of resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include a reduction in the number, size, and/or density of lesions, change in the color of lesions (such as from a tan coloration to a reddish brown coloration), reduction in number and density of pustule formation, reduction in sporulation, or any combination thereof.

In embodiments, the nucleic acid of the present invention, encoding a protein conferring enhanced pathogen resistance when expressed in a plant cell, herein also referred to as a resistance gene or R-gene, can used to enhance pathogen resistance to a fungal pathogen and/or a nematode. As non-limiting examples, the R-gene of the present invention can be used to enhance resistance to: soy cyst nematode, bacterial pustule, root knot nematode, frog eye leaf spot, phytophthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme.

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean.

An “elite” plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

The terms “agronomically elite” as used herein, means a genotype that has a culmination of many distinguishable traits such as emergence, vigor, vegetative vigor, disease resistance, seed set, standability, yield and threshability which allows a producer to harvest a product of commercial significance.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in, or endogenous to, the organism.

The terms “nucleic acid,” “nucleic acid molecule,” “nucleotide sequence,” “oligonucleotide”, “polynucleic acids” and “polynucleotide” are used interchangeably herein, unless the context indicates otherwise, and refer to a heteropolymer of nucleotides. These terms include without limitation DNA and RNA molecules, including cDNA, genomic DNA, synthetic (e.g., chemically synthesized) DNA and RNA, plasmid DNA, mRNA, anti-sense RNA, and RNA/DNA hybrids, any of which can be linear or branched, single stranded or double stranded, or a combination thereof. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. In embodiments, the “nucleic acid,” “nucleic acid molecule,”, “nucleotide sequence,”, “oligonucleotide” or “polynucleotide” refer to DNA.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence, means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to or “operatively associated” with the nucleotide sequence.

As used herein, the terms “disease tolerance” and “disease resistant” refer to a plant's ability to endure and/or thrive despite being infected with a respective disease. When used in reference to germplasm, the terms refer to the ability of a plant that arises from that germplasm to endure and/or thrive despite being infected with a respective disease. In some embodiments, infected disease resistant soybean plants may yield as well (or nearly as well) as uninfected soybean plants. In general, a plant or germplasm is labeled as “Disease resistant” if it displays “enhanced pathogen resistance.”

As used herein, the term “endogenous” or “native” refers to materials originating from within an organism or cell. In contrast, “heterogenous” or “heterologous” or “exogenous” refers to materials not originating naturally from within the organism or cell, due to modifications being artificially introduced to their endogenous state. This typically applies to nucleic acid molecules used in producing transformed or transgenic host cells and plants. For example, a nucleic acid molecule comprising the R-gene of the present invention is an exogenous nucleic acid used to confer or enhance pathogen resistance in a plant cell transformed with the nucleic acid molecule.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, a “genetic map” is a description of genetic linkage relationships among loci on one or more chromosomes within a given species, generally depicted in a diagrammatic or tabular form. For each genetic map, distances between loci are measured by the recombination frequencies between them. Recombinations between loci can be detected using a variety of markers. A genetic map is a product of the mapping population, types of markers used, and the polymorphic potential of each marker between different populations. The order and genetic distances between loci can differ from one genetic map to another.

As used herein, the term “genome” as it applies to plant cells encompasses not only chromosomal DNA found within the nucleus, but organelle DNA found within subcellular components of the cell.

The term “gene” or “genomic sequence” means a nucleic acid that comprises chromosomal DNA, genomic DNA, plasmid DNA, cDNA, an artificial DNA polynucleotide, or other DNA encoding a polypeptide of interest. In particular embodiments, the nucleic acid sequence of the gene encodes a protein that, when expressed, is responsible, at least in part, for a particular characteristic or trait. In embodiments, the gene may be native, modified (e.g., by directed recombination or site-specific mutation), or synthetic. In example embodiments, the gene is transcribed into an RNA molecule (e.g., an mRNA) in a cell wherein the RNA may encode a peptide, polypeptide, or protein of interest, and in some examples may also encode genetic elements flanking the coding sequence that are involved in the regulation of expression of the mRNA or polypeptide of the present invention. A gene may thus comprise several operably linked sequences, such as a promoter sequence, a 5′ leader sequence comprising, for example, sequences involved in translation initiation, a (protein) coding region (comprising cDNA or genomic DNA), a 3′ non-translated sequence comprising, for example, transcription termination sequence sites, introns (e.g., one or more native, foreign, or modified introns). In example embodiments, the nucleic acid sequence of the isolated gene may include introns, exons, 5′ or 3′-untranslated regions (UTRs), and native regulatory elements (such as native promoters). In other example embodiments, the gene comprises a coding sequence for a polypeptide of interest without including any regulatory elements (e.g., without any native or foreign introns, with some native introns replaced with foreign or modified introns, without any untranslated sequences, or with native regulatory elements replaced with foreign, heterologous or modified regulatory elements).

A “fragment” of a gene or nucleic acid is a portion of a full-length nucleic acid molecule that is of at least a minimum length capable of transcription into an RNA, translation into a peptide, or useful as a probe or primer in a DNA detection method. A “functional fragment” of a gene or nucleic acid is a portion of the full-length nucleic acid molecule that is capable of performing the same function as the full-length nucleic acid molecule. In embodiments, a functional fragment of a chromosomal interval that confers increased pathogen resistance includes a gene derived from the chromosomal interval.

The terms “nucleic acid,” “nucleic acid molecule,” and “polynucleotide” are used interchangeably herein. In embodiments, the gene is a segment of single-stranded, double-stranded or partially double-stranded DNA or RNA, or a hybrid thereof, that can be isolated or synthesized from any source. In the context of the present disclosure, the gene is typically a segment of DNA. In some embodiments, the gene of the disclosure includes isolated nucleic acid molecules. In some embodiments, the gene of the disclosure is comprised within a vector, expression cassette, a plant, or a plant cell.

As used herein, in particular embodiments, “R-gene” or “Resistance gene” refers to a nucleic acid having a nucleotide sequence (e.g., DNA sequence) encoding a polypeptide of interest, R-protein, or Resistance protein, that when expressed in a plant cell, confers to the plant cell, and/or the plant comprising the plant cell, with increased resistance to one or more plant pathogens. For example, in embodiments, the R-gene(s) of the present disclosure encode polypeptides or R-proteins that, when expressed in a soybean plant cell, confer the soybean plant with resistance to at least Asian Soybean Rust. In embodiments, the R-gene may comprise one or more motifs that correlate with one or more domains of the corresponding R-protein. For example, embodiments of the R-gene may comprise a TNL motif comprising a Toll/Interleukin-1 receptor (TIR) motif, a nucleotide-binding site (NBS), and a leucine rich-repeat (LRR) motif. When expressed, the TNL motif encodes a TNL motif in the R-protein comprising a Toll/Interleukin-1 receptor (TIR) domain a nucleotide-binding site (NBS) domain, and a leucine rich-repeat (LRR) domain. In other embodiments, the R-gene may comprise a CNL motif comprising a coiled coil (CC) motif, a nucleotide-binding site (NBS), and a leucine rich-repeat (LRR) motif. When expressed, the CNL motif encodes a CNL motif in the R-protein comprising a coiled coil (CC) domain, a nucleotide-binding site (NBS) domain, and a leucine rich-repeat (LRR) domain. The R-gene may comprise still other domains and motifs, such as a WRKY motif. In embodiments, the nucleic acid sequence of the R-gene is derived from a wild plant exhibiting increased resistance to the pathogen and includes, at least a coding sequence encoding the R-protein. The nucleic acid sequence of the R-gene may further comprise nucleic acid sequences corresponding to one or more native regulatory elements (such as native introns, native promoters, native UTRs), one or more heterologous regulatory elements (such as a heterologous promoter and introns), and combinations thereof. Insertion of the R-gene into a plant that has decreased resistance to the pathogen (e.g., no resistance or partially or fully susceptible), at a chromosomal location (e.g., stably integrated into the plant genome) or extra-chromosomal location (e.g., on a vector or plasmid) results in conferring of the wild plant-derived pathogen resistance to the recipient plant. For example, in representative embodiments, an R-gene of the present invention is derived from Glycine tomentella and can be inserted into Glycine max plants to confer or enhance resistance of G. max plants to Asian Soy Rust.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific molecular makeup that provides a physical foundation for some or all of the hereditary qualities of an organism or cell culture. As used herein, germplasm may refer to seeds, cells (including protoplasts and calli) or tissues from which new plants may be grown, as well as plant parts that can be cultured into a whole plant (e.g., stems, buds, roots, leaves, etc.).

As used herein, a “Heterologous DNA” sequence refers to a polynucleotide sequence that originates from a foreign source or species or, if from the same source, is modified from its original form.

As used herein, a “Homologous DNA” refers to DNA from the same source as that of the recipient cell.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the term “indel” refers to an insertion or deletion in a pair of nucleotide sequences, wherein a first sequence may be referred to as having an insertion relative to a second sequence or the second sequence may be referred to as having a deletion relative to the first sequence.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, an R-gene or marker associated with enhanced ASR tolerance or resistance may be introgressed from a donor into a recurrent parent that is not disease resistant. The resulting offspring could then be repeatedly backcrossed and selected until the progeny possess the ASR tolerance allele(s) in the recurrent parent background.

As used herein, an “isolated” nucleic acid molecule or gene is substantially separated away from other nucleic acid or gene sequences with which the nucleic acid is normally associated, such as, from the chromosomal or extrachromosomal DNA of a cell in which the nucleic acid or gene naturally occurs. A nucleic acid molecule is an isolated nucleic acid molecule when it comprises a transgene or part of a transgene present in the genome of another organism. The term also embraces nucleic acids that are biochemically purified to substantially remove contaminating nucleic acids and other cellular components.

A polypeptide is “isolated” if it has been separated from the cellular components (nucleic acids, lipids, carbohydrates, and other polypeptides) that naturally accompany it or that is chemically synthesized or recombinant. A polypeptide molecule is an isolated polypeptide molecule when it is expressed from a transgene in another organism. A monomeric polypeptide is isolated when at least 60% by weight of a sample is composed of the polypeptide, preferably 90% or more, more preferably 95% or more, and most preferably more than 99%. Protein purity or homogeneity is indicated, for example, by polyacrylamide gel electrophoresis of a protein sample, followed by visualization of a single polypeptide band upon staining the polyacrylamide gel; high pressure liquid chromatography; or other conventional methods. Proteins can be purified by any of the means known in the art, for example as described in Guide to Protein Purification, ed. Deutscher, Meth. Enzymol. 185, Academic Press, San Diego, 1990; and Scopes, Protein Purification: Principles and Practice, Springer Verlag, New York, 1982.

Using well-known methods, the skilled artisan can readily produce nucleotide and amino acid sequence variants of genes and proteins that provide a modified gene product. Chemical synthesis of nucleic acids can be performed, for example, on automated oligonucleotide synthesizers. Such variants preferably do not change the reading frame of the protein-coding region of the nucleic acid. The present invention also encompasses fragments of a protein that lacks at least one residue of a full-length protein, but that substantially maintains activity of the protein.

A “locus” is a position on a chromosome where a gene or marker or allele is located. In some embodiments, a locus may encompass one or more nucleotides.

A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm and crossing a naturally occurring variety of soybean with a non-naturally occurring variety of soybean. In some embodiments, a “non-naturally occurring variety of soybean” may comprise one of more heterologous nucleotide sequences. In some embodiments, a “non-naturally occurring variety of soybean” may comprise one or more non-naturally occurring copies of a naturally occurring nucleotide sequence (i.e., extraneous copies of a gene that naturally occurs in soybean). In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines such as polynucleotides from wild glycine species).

As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, or an electromechanical assay. In some cases, a phenotype or trait is directly controlled by a single gene or genetic locus, i.e., a “single gene trait.” In other cases, a phenotype or trait is the result of several genes. It is noted that, as used herein, the term “pathogen resistant phenotype” or “disease resistant phenotype” takes into account environmental conditions that might affect the respective pathogen or disease such that the effect is real and reproducible.

As used herein, the term “plant” may refer to a whole plant, any part thereof, or a cell or tissue culture derived from a plant. Thus, the term “plant” can refer to any of: whole plants, plant components or organs (e.g., roots, stems, leaves, buds, flowers, pods, etc.), plant tissues, seeds and/or plant cells. A plant cell is a cell of a plant, taken from a plant, or derived through culture from a cell taken from a plant. Thus, the term “soybean plant” may refer to a whole soybean plant, one or more parts of a soybean plant (e.g., roots, root tips, stems, leaves, buds, flowers, pods, seeds, cotyledons, etc.), soybean plant cells, soybean plant protoplasts and/or soybean plant calli.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. In embodiments, the plant cell is non-propagating and/or cannot regenerate a whole plant.

A “plant cell culture” means a culture of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

As used herein, the term “plant part” includes but is not limited to embryos, pollen, ovules, seeds, leaves, flowers, branches, fruit, stalks, roots, root tips, anthers, and/or plant cells including plant cells that are intact in plants and/or parts of plants, plant protoplasts, plant tissues, plant cell tissue cultures, plant calli, plant clumps, and the like.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural or functional units. The use of this term in conjunction with, or in the absence of, any specific type of plant tissue as listed above or otherwise embraced by this definition is not intended to be exclusive of any other type of plant tissue.

“Polyadenylation signal” or “polyA signal” refers to a nucleic acid sequence located 3′ to a coding region that causes the addition of adenylate nucleotides to the 3′ end of the mRNA transcribed from the coding region.

“Polymerase chain reaction (PCR)” refers to a DNA amplification method that uses an enzymatic technique to create multiple copies of one sequence of nucleic acid (amplicon). Copies of a DNA molecule are prepared by shuttling a DNA polymerase between two amplimers. The basis of this amplification method is multiple cycles of temperature changes to denature, then re-anneal amplimers (DNA primer molecules), followed by extension to synthesize new DNA strands in the region located between the flanking amplimers. Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066. Primers can be labeled, if desired, by incorporating detectable moieties by for instance spectroscopic, fluorescence, photochemical, biochemical, immunochemical, or chemical moieties. Primers diagnostic (i.e. able to identify or select based on presence of ASR resistant alleles) for ASR resistance can be created to any favorable SNP as described in any one of Tables 1-5. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein, “protein” refers to a polynucleotide, usually upstream (5′) of its coding polynucleotide, which controls the expression of the coding polynucleotide by providing the recognition for RNA polymerase and other factors required for proper transcription. In example embodiments of the present invention, proteins or polynucleotides are provided that when expressed in a plant or plant cell, confer the plant with enhanced resistance to a plant pathogen.

The term “promoter” or “promoter region” refers to a polynucleic acid molecule that functions as a regulatory element, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant DNA construct, as demonstrated by its ability to produce mRNA. In some embodiments, such as in the vector constructs and expression cassettes disclosed herein, the promoter may be heterologous to the coding sequence whose expression the promoter controls, such as when the promoter and the coding sequence are derived from different sources, such as different organisms (e.g., in embodiments, the vectors described herein comprise an R-gene sequence derived from Glycine tomentella and a promoter sequence derived from Medicago trunculata). In other examples, the promoter may be endogenous or native to the coding sequence whose expression the promoter controls, such as when the promoter and the coding sequence are derived from a common source, such as a common organism. A number of promoters can be used in an expression cassette, including a combination of the native promoter of an R gene encoding an R protein and one or more heterologous promoters.

Alternatively, promoters can be selected based upon a desired outcome. Such promoters include, but are not limited to, “constitutive promoters” (where expression of a polynucleotide sequence operably linked to the promoter is unregulated and therefore continuous), “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed by an analyte, cofactor, regulatory protein, etc.), and “tissue-preferred promoters” (where expression of a polynucleotide sequence operably linked to the promoter is higher in a preferred tissue relative to other tissues, such as higher in leaf tissue relative to other plant tissues).

As used herein, “plant promoter” means a promoter that drives expression in a plant such as a constitutive, inducible (e.g., chemical-, environmental-, pathogen- or wound-inducible), repressible, tissue-preferred or other promoter for use in plants.

Example promoters are set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference. Example constitutive promoters include CaMV 35S promoter (Odell et al. (985) Nature 313:810-812); rice actin (McElroy et al. (1990) Plant Cell 2: 163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81: 581-588); MAS (Velten e/a/. (1984) EMBO J. 3:2723-2730). Example inducible promoters include those that drive expression of pathogenesis-related proteins (PR proteins), which are induced following infection by a pathogen. See, for example, Redolfi et al. (1983) Neth. J. Plant Pathol. 89:245-254; Uknes et al. (1992) Plant Cell 4:645-656; and Van Loon (1985) Plant Mol. Virol. 4: 111-116; and WO 99/43819, herein incorporated by reference. Promoters that are expressed locally at or near the site of pathogen infection may also be used (Marineau et al. (1987) Plant Mol. Biol. 9:335-342; Matton et al. (1989) Molecular Plant-Microbe Interactions 2: 325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93: 14972-14977; Chen et al. (1996) Plant J. 10:955-966; Zhang et al. (1994) Proc. Natl. Acad. Sci. USA 91:2507-2511; Warner et al. (1993) Plant J. 3: 191-201; Siebertz et al. (1989) Plant Cell 1:961-968; Cordero et al. (1992) Physiol. Mol. Plant Path. 41: 189-200; U.S. Pat. No. 5,750,386 (nematode-inducible); and the references cited therein).

Wound-inducible promoters include pin II promoter (Ryan (1990) Ann. Rev. Phytopath. 28:425-449; Ouan et al. (1996) Nature Biotechnology 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225: 1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2): 141-150); and the like, herein incorporated by reference.

Tissue-preferred promoters for use in the invention include those set forth in Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascim et al. (1996) Plant Physiol. 112(2):513-524; Yamamoto et al. (1994) Plant Cell Physiol. 35(5):773-778; Lam (1994) Results Probl. Cell Differ. 20: 181-196; Orozco et al. (1993) PlantMolBiol. 23(6): 1129-1138; Matsuoka et al. (1993) Proc Natl. Acad. Sci. USA 90(20):9586-9590; and Guevara-Garcia et al. (1993) Plant J. 4(3):495-505.

Leaf-preferred promoters include those set forth in Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20):9586-9590.

Root-preferred promoters are known and include those in Hire et al. (1992) Plant Mol. Biol. 20(2):207-218 (soybean root-specific glutamine synthetase gene); Keller and Baumgartner (1991) Plant Cell 3(10): 1051-1061 (root-specific control element); Sanger et al. (1990) Plant Mol. Biol. 14(3):433-443 (mannopine synthase (MAS) gene of Agrobacterium tumefaciens); and Miao et al. (1991) Plant Cell 3(1): 11-22 (cytosolic glutamine synthetase (GS)); Bogusz et al. (1990) Plant Cell 2(7):633-641; Leach and Aoyagi (1991) Plant Science (Limerick) 79(1):69-76 (rolC and rolD); Teeri et al. (1989) EMBO J. 8(2):343-350; Kuster et al. (1995) Plant Mol. Biol. 29(4):759-772 (the VfENOD-GRP3 gene promoter); and, Capana et al. (1994) Plant Mol. Biol. 25(4):681-691 (rolB promoter). See also U.S. Pat. Nos. 5,837,876; 5,750,386; 5,633,363; 5,459,252; 5,401,836; 5,110,732; and 5,023,179.

As used herein, “recombinant” refers to a form of nucleic acid (e.g., DNA or RNA) and/or protein and/or an organism that would not normally be found in nature and as such was created by human intervention. Such human intervention may produce a recombinant nucleic acid molecule and/or a recombinant plant. As used herein, a “recombinant DNA molecule” is a DNA molecule comprising a combination of DNA molecules that would not naturally occur together and is the result of human intervention, e.g., a DNA molecule that is comprised of a combination of at least two DNA molecules heterologous to each other, and/or a DNA molecule that is artificially synthesized and comprises a polynucleotide that deviates from the polynucleotide that would normally exist in nature, and/or a DNA molecule that is artificially incorporated into a host cell's genomic DNA and the associated flanking DNA of the host cell's genome. An example of a recombinant DNA molecule is a DNA molecule resulting from the insertion of the transgene or a genome modification (i.e., a gene edit) into a plant's genomic DNA, which may ultimately result in the expression of a recombinant RNA and/or protein molecule in that organism. As used herein, a “recombinant plant” is a plant that would not normally exist in nature, is the result of human intervention, and contains a transgene and/or heterologous DNA molecule and/or a genome modification (i.e., a gene edit) incorporated into its genome. As a result of such genomic alteration, the recombinant plant is distinctly different from the related wildtype plant. The term “recombinant DNA construct” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a DNA molecule that one or more DNA sequences have been linked in a functionally operative manner. Recombinant DNA constructs may be constructed to be capable of expressing antisense RNAs or stabilized double stranded antisense RNAs.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using a sequence comparison algorithm or by visual inspection. In certain embodiments, substantially identical sequences have at least about 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or more nucleotide or amino acid residue identity. In certain embodiments, substantial identity exists over a region of the sequences that is at least about 50 amino acid residues, 100 amino acid residues, 150 amino acid residues, 200 amino acid residues, 250 amino acid residues, 300 amino acid residues, 350 amino acid residues, 400 amino acid residues, 450 amino acid residues, 500 amino acid residues, 525 amino acid residues, 526, amino acid residues 527 amino acid residues, 528 amino acid residues, 529 amino acid residues, 530 amino acid residues, 531 amino acid residues, 532 amino acid residues, 533 amino acid residues, 534 amino acid residues, 535 amino acid residues, 536 amino acid residues or more with respect to the protein sequence or the nucleotide sequence encoding the same. In further embodiments, the sequences are substantially identical when they are identical over the entire length of the coding regions.

The term “identity”, “sequence identity”, “homology”, “similarity”. “sequence similarity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/ and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In a preferred embodiment, substantially identical nucleic acid or amino acid sequences may perform substantially the same function. In embodiments, sequences that are substantially identical have at least 60%, at least 70%, at least 80%, at least 85%, at least 90%, or at least 95% sequence identity to each other.

The terms “homology”, “sequence similarity”, or “sequence identity” in reference to nucleotide or amino acid sequences mean a degree of identity or similarity of two or more sequences and may be determined conventionally by using known software or computer programs such as the Best-Fit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2:482-489 (1981), to find the best segment of identity or similarity between two sequences. Sequence comparison between two or more polynucleotides or polypeptides is generally performed by comparing portions of the two sequences over a comparison window to identify and compare local regions of sequence similarity. The comparison window is generally from about 20 to 200 contiguous nucleotides. Gap performs global alignments: all of one sequence with all of another similar sequence using the method of Needleman and Wunsch, J. Mol. Biol. 48:443-453 (1970). When using a sequence alignment program such as BestFit to determine the degree of DNA sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions.

The terms “complementary” or “complementarity” (and similar terms), as used herein, refer to the natural binding of polynucleotides under permissive salt and temperature conditions by base-pairing. For example, the sequence “A-G-T” binds to the complementary sequence “T-C-A.” Complementarity between two single-stranded molecules may be partial, in which only some of the nucleotides bind, or it may be complete when total complementarity exists between the single stranded molecules. The degree of complementarity between nucleic acid strands has significant effects on the efficiency and strength of hybridization between the molecules. As used herein, the term “substantially complementary” (and similar terms) means that two nucleic acid sequences are at least about 50%, 60%, 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein).

As used herein, “specifically” or “selectively” hybridizing (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Specifically or selectively hybridizing sequences typically are at least about 40% complementary and are optionally substantially complementary or even completely complementary (i.e., 100% identical).

For DNA-DNA hybrids, the Tₘ can be approximated from the equation of Meinkoth and Wahl, Anal. Biochem., 138:267-84 (1984): Tₘ=81.5° C.+16.6 (log M)+0.41 (% GC)−0.61 (% formamide)−500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired degree of identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, highly stringent conditions can utilize a hybridization and/or wash at the thermal melting point (Tₘ) or 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). If the desired degree of mismatching results in a Tₘ of less than 45° C. (aqueous solution) or 32° C. (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37° C. and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1 M NaCl, 1% SDS at 37° C. and a wash in 0.5× to 1×SSC at 55° C. to 60° C. Exemplary high stringency conditions include hybridization in 50% formamide, 1 M NaCl, 1% SDS at 37° C. and a wash in 0.1×SSC at 60° C. to 65° C. A further non-limiting example of high stringency conditions include hybridization in 4×SSC, 5×Denhardt's, 0.1 mg/ml boiled salmon sperm DNA, and 25 mM Na phosphate at 65° C. and a wash in 0.1×SSC, 0.1% SDS at 65° C. Another illustration of high stringency hybridization conditions includes hybridization in 7% SDS, 0.5 M NaPO₄, 1 mM EDTA at 50° C. with washing in 2×SSC, 0.1% SDS at 50° C., alternatively with washing in 1×SSC, 0.1% SDS at 50° C., alternatively with washing in 0.5×SSC, 0.1% SDS at 50° C., or alternatively with washing in 0.1×SSC, 0.1% SDS at 50° C., or even with washing in 0.1×SSC, 0.1% SDS at 65° C. Those skilled in the art will appreciate that specificity is typically a function of post-hybridization washes, the relevant factors being the ionic strength and temperature of the final wash solution.

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions.

The term “vector” refers to a composition for transferring, delivering or introducing a nucleic acid (or nucleic acids) into a cell. A vector comprises a nucleic acid molecule comprising the nucleotide sequence(s) to be transferred, delivered or introduced.

Provided herein are plants expressing polypeptides that increase the plant's ability for pathogen resistance as compared to a control plant that does not express the polypeptide. The polypeptide includes SEQ ID NO: 5 and functional fragments and variants thereof. Various means of introducing nucleic acid sequence into the soybean plant are also disclosed, which include transgenic means, gene editing, and breeding. Markers for identifying the presence of these nucleic acid sequences in the plant are also disclosed.

In some embodiments, the plants provided herein are a non-naturally occurring variety of soybean having the desired trait. In specific embodiments, the non-naturally occurring variety of soybean is an elite soybean variety. A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm and crossing a naturally occurring variety of soybean with a non-naturally occurring variety of soybean. In some embodiments, a “non-naturally occurring variety of soybean” may comprise one of more heterologous nucleotide sequences. In some embodiments, a “non-naturally occurring variety of soybean” may comprise one or more non-naturally occurring copies of a naturally occurring nucleotide sequence (i.e., extraneous copies of a gene that naturally occurs in soybean). In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines).

Methods and compositions are provided that increase the pathogen resistance capability in a plant, a plant part, or a seed. In specific embodiments, various methods and compositions are provided that produce an increase in resistance to Asian Soy Rust in the plant, plant part or seed. An increase in pathogen resistance includes any statistically significant increase in plant's ability to resist infection by the pathogen when compared to an appropriate control plant or plant part.

A “subject plant or plant cell” is one in which genetic alteration, such as transformation, has been affected as to a polynucleotide of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Expression of Polynucleotides and Polypeptides that Confer Increased Pathogen Resistance

Compositions and methods for conferring increased pathogen resistance are provided. Polypeptides, polynucleotides and functional fragments and variants thereof that confer increased pathogen resistance are provided. In some embodiments, the polypeptide is SEQ ID NO: 5 or a fragment or variant of SEQ ID NO: 5. In some embodiments, the polynucleotide is any one of SEQ ID NOS: 1, 2-4, 11 and 12 or a polynucleotide encoding a polypeptide having the sequence of SEQ ID NO: 5, or a fragment or variant of any one thereof. In various embodiments, the polypeptide and polynucleotides or variant and fragments thereof confer increased resistance to Asian Soy Rust.

Fragments of the polypeptides that increase pathogen resistance when expressed in a plant, plant part, or seed include those that are shorter than the full-length sequences, either due to the use of an alternate downstream start site, or due to processing that produces a shorter protein having the activity. Such biologically active portions can be prepared by recombinant techniques and evaluated for activity of being able to confer increased pathogen resistance.

Variants disclosed herein include polypeptides having an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 5. Similarly, variants disclosed herein include polynucleotides having a nucleotide sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the nucleotide sequence of any of SEQ ID NOS: 1, 2-4 and 11-12. Such variants will increase pathogen resistance when expressed in a plant, plant part or seed. In some embodiments, a variant polynucleotide/polypeptide comprises a deletion and/or addition of one or more nucleotides/amino acids at one or more internal sites within the native polynucleotide/polypeptide and/or a substitution of one or more nucleotides/amino acids at one or more sites in the native polynucleotide/polypeptide.

In some embodiments, the polypeptides disclosed herein may comprise a heterologous amino acid sequence attached thereto. For example, a polypeptide may have a polypeptide tag or additional protein domain attached thereto. The heterologous amino acid sequence can be attached to the N terminus, the C terminus, or internally within the polypeptide. In some instances, the polypeptide may have one or more polypeptide tags and/or additional protein domains attached thereto at one or more positions of the polypeptide.

In some embodiments, the nucleic acid sequence encoding the polypeptides disclosed herein may comprise a heterologous nucleic acid sequence attached thereto. For example, the heterologous nucleic acid sequence may encode a polypeptide tag or additional protein domain that will be attached to the encoded polypeptide. As another example, the heterologous nucleic acid sequence may encode a regulatory element such as an intron, an enhancer, a promoter, a terminator, etc. The heterologous nucleic acid sequence can be positioned at the 5′ end, the 3′ end, or in-frame within the coding sequence of the polypeptide. In some instances, the nucleic acid sequence encoding the polypeptides disclosed herein may have one or more heterologous nucleic acid sequences attached thereto at one or more positions of the nucleic acid sequence. In still other embodiments, nucleotide sequences disclosed herein further comprise one or more native regulatory elements, including, for example, the native promoter sequence, the native 5′UTR, the native 3′UTR and/or the native terminator, or any combination thereof.

Polynucleotides encoding the polypeptides provided herein can be provided in expression cassettes (herein also referred to as “DNA constructs”) for expression in an organism of interest. The cassette will include 5′ and 3′ regulatory sequences operably linked to a polynucleotide encoding a polypeptide provided herein that allows for expression of the polynucleotide comprising an R-gene in plants, thereby imparting pathogen resistance to the plants in which they are expressed. The cassette may additionally contain at least one additional gene or genetic element to be co-transformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory elements or regions. The expression cassette may additionally contain a selectable marker gene.

“DNA construct” refers to the genetic elements operably linked to each other making up a recombinant DNA molecule and may comprise elements that provide expression of a DNA polynucleotide molecule in a host cell and elements that provide maintenance of the construct in the host cell. The various genetic elements within the DNA construct can be native to polynucleotide encoding the polypeptide or heterologous to the native polynucleotide encoding the polypeptide. A plant expression cassette comprises the operable linkage of genetic elements that when transferred into a plant cell provides expression of a desirable gene product. “Plant expression cassette” refers to chimeric DNA segments comprising the regulatory elements that are operably linked to provide the expression of a transgene product in plants. Promoters, leaders, introns, transit peptide encoding polynucleic acids, 3′ transcriptional termination regions are all genetic elements that may be operably linked by those skilled in the art of plant molecular biology to provide a desirable level of expression or functionality to an R-gene of the present invention. A DNA construct can contain one or more plant expression cassettes expressing the DNA molecules of the present invention or other DNA molecules useful in the genetic engineering of crop plants. One example of a DNA construct that may be used for expressing the R-gene of the present invention is a vector with a nucleic acid sequence comprising the R-gene operably linked to one or more heterologous regulatory elements (e.g., a heterologous promoter and/or intron) or native regulatory elements (e.g., native promoter, introns, or terminator regions).

The expression cassette will include in the 5′-3′ direction of transcription, a transcriptional and translational initiation region (i.e., a promoter), a polynucleotide of the invention, and a transcriptional and translational termination region (i.e., termination region) functional in the organism of interest, i.e., a plant or bacteria. The promoters of the invention are capable of directing or driving transcription and expression of a coding sequence in a host cell. The regulatory regions (i.e., promoters, transcriptional regulatory regions, and translational termination regions) may be endogenous or heterologous to the host cell or to each other. As used herein, a chimeric gene or a chimeric nucleic acid molecule comprises a coding sequence operably linked to a transcription initiation region that is heterologous to the coding sequence.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. Still other terminators that can be used include heterologous terminators derived from Arabidopsis genes, such as the terminators of SEQ ID NOS: 16-17. In addition, a gene's native transcription terminator may be used. In one example embodiment, the native transcription terminator of the nucleic acid (R-gene) encoding a polypeptide conferring increased pathogen resistance is used, such as the native terminator of SEQ ID NO: 18. Termination regions used in the expression cassettes can also be obtained from, e.g., the Ti-plasmid of A. tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (990) Plant Cell 2: 1261-1272; Munroe et al. (1990) Gene 91: 151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al, eds. (1980).

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved A variety of promoters that are constitutively active or specifically active in vegetative tissues, such as leaves, stems, roots and tubers, can be used to express the nucleic acid or R-gene of the present invention. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference. In some embodiments, the promoter used to drive the expression of the polynucleotides provided herein comprises an exogenous promoter not found in plants in nature, for example, a synthetic promoter. In some embodiments, the promoter used to drive the expression of the polynucleotides provided herein comprises a heterologous promoter sourced from an organism that is different from the organism from where the R-gene is sourced. In example embodiments, the nucleic acid comprising the R-gene is expressed in soybean plants wherein the expression is driven by a plant promoter derived from Medicago truncatula, such as the promoters of SEQ ID NOS: 13-14.

In embodiments, the promoter may also optionally comprise an intron. In some embodiments, a promoter comprises or consists of the about 2 kb region upstream (5′) of the translation start site of a known or predicted coding sequence. In other embodiments, the promoter is a minimal or core promoter comprising only those elements that are required to initiate transcription. For example, a minimal promoter may consist of a transcription start site (TSS), a binding site for RNA polymerase, and a transcription factor binding site (such as a TATA box or B recognition element). Such minimal promoter may not comprise any introns or splice sites.

In some embodiments, the promoter used herein to drive the expression of the polynucleotides provided herein comprises a native promoter or an active variant or fragment thereof. For purpose of this disclosure, the term “native promoter,” used interchangeably with the term “endogenous promoter,” refers to a promoter that is found in plants in nature. An active variant or fragment of a native promoter refers to a promoter sequence that has one or more nucleotide substitutions, deletions, or insertions and that can drive expression of an operably-linked polynucleotide sequence under conditions similar to those under which the native promoter is active. Such active variants or fragments may be created by site-directed mutagenesis, induced mutation, or may occur as allelic variants (polymorphisms). In some embodiments, the native promoter comprises a polynucleotide having the sequence of SEQ ID NO: 15. In some embodiments, disclosed herein is a construct comprising a native promoter (e.g., a native promoter comprising SEQ ID NO: 15) or its active variant or fragment operably linked to a polynucleotide encoding a polypeptide having the sequence of SEQ ID NO: 5, or a fragment or variant of SEQ ID NO: 5 (e.g., having least 85%, 90%, 91%, 92%, 93%, 94%, 95%, 96%, 97%, 98%, or 99% identity to the native promoter, wherein the variant or fragment thereof retains the ability to direct expression of a sequence of interest); and when introduced into a plant, the construct confers increased pathogen resistance. In some embodiments, the native promoter is a heterologous promoter to the polynucleotide.

The translation leader sequence means a DNA molecule located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders, plant virus coat protein leaders, plant rubisco gene leaders among others (Turner and Foster, Molecular Biotechnology 3:225, 1995).

The “3′ non-translated sequences” (or 3′ untranslated sequences or 3′-UTR) means DNA sequences located downstream of a structural polynucleotide sequence and include sequences encoding polyadenylation and other regulatory signals capable of affecting mRNA processing or gene expression. The polyadenylation signal functions in plants to cause the addition of multiple adenylate nucleotides to the 3′ end of the mRNA precursor. The polyadenylation sequence can be derived from the natural gene, from a variety of plant genes, or from T-DNA. An example of the polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989. In embodiments of the present invention, the nucleic acid encoding a polypeptide conferring increased pathogen resistance comprises the native or endogenous 3′-UTR of the R-gene, such as the 3′-UTR of SEQ ID NO: 20.

The “5′ non-translated sequences” (or 5′ untranslated sequences or 5′-UTR) means DNA sequences located upstream of an initiation codon of structural polynucleotide sequence and include sequences capable of affecting translation of an mRNA sequence. The 5′-UTR sequence is also referred to as a leader sequence. In different organisms, the 5′-UTR may remain untranslated, and form complex secondary structures to regulate translation of the downstream sequence. The leader sequence can be derived from the natural gene or from a variety of plant genes. In embodiments of the present invention, the nucleic acid encoding a polypeptide conferring increased pathogen resistance comprises the native or endogenous 5′-UTR of the R-gene, such as the 5′-UTR of SEQ ID NO: 19.

As used herein, the term “intron” refers to a nucleotide sequence provided within a gene (that is, in an intragenic region) and that is removed by splicing during maturation of a final RNA product. Thus, introns are non-coding regions of an RNA transcript, or the DNA encoding it. Introns may have regulatory function, such as due to the presence of transcriptional enhancer or repressor sequences embedded therein. Example introns include introns derived from Arabidopsis genes, such as the intron of SEQ ID NO: 21. Introns separate exons such that splicing results in removal of introns and joining of exons. Introns are marked by the presence of conserved sequences known as splice sites at 5′ and 3′ ends. Typically, the splice site at the 5′ end includes an AG sequence and the splice site at the 3′ end includes a GU sequence. Splicing of the introns is catalyzed by a spliceosome comprising RNA and proteins. Promoter sequences can, in some embodiments (e.g., for larger promoter sequences such as those for proximal or distal promoters) include an intron. In other embodiments, as described herein, introns may be optionally coupled to a minimal or core promoter sequence, as well as to a nucleic acid encoding a protein of interest, to improve expression of the protein.

This in embodiments, novel regulatory elements are disclosed for the expression of polynucleotides and polypeptides in a plant cell. In particular embodiments, the novel regulatory elements are used for the expression of polynucleotides and polypeptides that when expressed in a plant cell confer the plant with increased pathogen resistance, such as to ASR. The novel regulatory elements include native promoters comprising the nucleotide sequence of SEQ ID NO: 15, or an active variant or fragment to SEQ ID NO: 15 (e.g., having at least 90%, 91%, 92%, 93%, 94%, 95%, 96%, 97%, 98%, or 99% sequence identity to SEQ ID NO: 15 and retaining the ability to drive expression of an operably linked polynucleotide of interest). In other embodiments, the promoter set forth in SEQ ID 15 is operably linked to a polynucleotide encoding the polypeptide of interest. In specific embodiments, the polynucleotide sequence of interest encodes a polypeptide that upon expression in a plant cell increases resistance of the plant cell to a plant pathogen, such as ASR. Such polypeptides include, but are not limited to, the polypeptide of SEQ ID NO: 5 or an active variant or fragment thereof or polypeptides encoding R-genes as set forth in any one of US Patent publication No. US 20200354739 and PCT Publications Nos. WO2019103918, WO2021154632A1, WO2021022022, WO2021022026, WO2021022101, WO2021260673, and WO2021263249, the contents of each of which are herein incorporated by reference in their entirety.

The novel regulatory elements further include native terminators comprising the nucleotide sequence of SEQ ID NO: 18, or a sequence that is substantially identical to SEQ ID NO: 18 (e.g., having at least 90% or at least 95% sequence identity). The novel regulatory elements further include native 5′- and 3′-UTRs comprising the nucleotide sequence of SEQ ID NO: 19 and/or 20, or a sequence that is substantially identical to SEQ ID NOS: 19 and/or 20 (e.g., having at least 90% or at least 95% sequence identity).

The polynucleotide of the invention comprises any coding sequence that can express the novel R-gene and encode a polypeptide that confers increased pathogen resistance. In particular embodiments, the coding sequence comprises any polynucleotide that encodes a polypeptide having the amino acid sequence of SEQ ID NO: 5, or an amino acid sequence having at least 90% or at least 95% sequence identity to SEQ ID NO: 5 and conferring increased pathogen resistance when expressed. In example embodiments, the coding sequence comprises a nucleic acid having the nucleotide sequence of any of SEQ ID NOS: 1, 2-4 and 11-12, or any nucleic acid having at least 90% or at least 95% sequence identity to any of SEQ ID NOS: 1, 2-4 and 11-12.

In example embodiments, the coding sequence is a cDNA derived nucleotide sequence that comprises the exons of the R-gene spliced together and without the sequence of intervening introns, or upstream and downstream UTRs (for example, the R-gene coding sequence of SEQ ID NO: 12). In other example embodiments, the coding sequence is a genomic DNA derived nucleotide sequence that comprises the exons with any combination of intervening native introns (e.g., one or more or all intervening introns), native 5′ and 3′ UTRs, native promoters and native terminators. One of skill in the art may be able to use standard procedures in recombinant DNA technology to combine any of the coding sequence options with native or exogenous terminators, native or exogenous promoters, and any combination of native or exogenous regulatory elements in the expression cassette.

In one embodiment, the genomic DNA derived coding sequence of the R-gene of the present invention comprises all the native introns and exons of the gene in addition to the native 5′ and 3′ UTRs and native promoters and terminators (for example, the R-gene coding sequence of SEQ ID NO: 2). In other example embodiments, one or more of the native introns are replaced with a non-native intron, such as with an intron known to enhance transformation, transcriptional, or translational activity in a host cell (for example, the R-gene coding sequences of SEQ ID NOS: 3-4 and 11-12 wherein the first native intron is replaced with the intron of SEQ ID NO: 21). In still further embodiments, the coding sequence comprises the native promoter and terminator as well as native UTRs allowing for the gene expression to be driven by the native promoter (for example, the R-gene coding sequences of SEQ ID NO: 11). In other embodiments, the coding sequence comprises the native UTRs but not the native promoter or terminator to allow for gene expression to be driven by a heterogenous promoter (for example, the R-gene coding sequences of SEQ ID NOS: 3-4).

The laboratory procedures in recombinant DNA technology used herein are those well-known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al. (1989).

The nucleic acids, polynucleotides, nucleotide sequences, R-genes, vectors and DNA constructs of the present invention may be introduced into the genome of a desired plant host by a variety of conventional transformation techniques that are well known to those skilled in the art. “Transformation” refers to a process of stably introducing an exogenous nucleic acid molecule (for example, a DNA construct, a vector, an expression cassette, or a recombinant polynucleic acid molecule) into a cell or protoplast and that exogenous nucleic acid molecule is stably incorporated into a host cell genome or an organelle genome (for example, chloroplast or mitochondria) or is capable of autonomous replication. “Transformed” or “transgenic” refers to a cell, tissue, organ, or organism into which a foreign polynucleic acid, such as a DNA vector or recombinant polynucleic acid molecule, is incorporated and maintained. Further, once stably transformed into a cell, the foreign polynucleic acid can be passed on to a progeny of the cell. A “transgenic”, “transformed”, or “stably transformed” cell or organism also includes progeny of the cell or organism and progeny produced from a breeding program employing such a “transgenic” plant as a parent in a cross and exhibiting an altered phenotype resulting from the presence of the foreign polynucleic acid molecule.

Methods of transformation of plant cells or tissues include but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3(7):637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

DNA constructs, vectors, and expression cassettes can be prepared that incorporate the R-gene coding sequences of the present invention for use in directing the expression of the sequences directly from the host plant cell plastid. Examples of such constructs suitable for this purpose and methods that are known in the art and are generally described, for example, in Svab et al., Proc. Natl. Acad. Sci. USA 87:8526-8530, (1990) and Svab et al., Proc. Natl. Acad. Sci. USA 90:913-917 (1993) and in U.S. Pat. No. 5,693,507.

When adequate numbers of cells containing the exogenous nucleic acid molecule encoding polypeptides from the present invention are obtained, the cells can be cultured, then regenerated into whole plants. “Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical See, for example, Ammirato et al., Handbook of Plant Cell Culture-Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

The development or regeneration of transgenic plants containing the exogenous polynucleic acid molecule that encodes a polypeptide of interest is well known in the art. Preferably, the regenerated plants are self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants.

In certain embodiments, the polynucleotides of the invention encoding a protein conferring enhanced pathogen resistance can be stacked with any combination of polynucleotide sequences of interest in order to create plants with a desired trait. A trait, as used herein, refers to the phenotype derived from a particular sequence or groups of sequences. For example, the polynucleotides encoding the novel R gene may be stacked with any other polynucleotides encoding polypeptides that confer a desirable trait, including but not limited to resistance to diseases, insects, and herbicides, tolerance to heat and drought, reduced time to crop maturity, improved industrial processing, such as for the conversion of starch or biomass to fermentable sugars, and improved agronomic quality, such as high oil content and high protein content.

In a particular embodiment of the invention, polynucleotides may be stacked (or, alternatively, multiple expression cassettes may be stacked on a single polynucleotide) so as to express more than one R-gene within a plant. This is a particular advantage where, for example, one R-gene is particularly suitable for providing resistance to one class of plant pathogens (e.g., a first rust isolate) while the other provides resistance to a different class of plant pathogens (or a different result isolate). In alternate embodiments, a first R-gene is provided that provides resistance via a first mode of action against a plant pathogen (e.g., against ASR) while the other provides resistance to the same plant pathogen via a second, different mode of action. Herein, the synergistic effect of the different modes of action of the different R-genes can provide a higher increase in overall pathogen resistance than either R-gene by itself. Stacking polypeptides encoded by different R-genes is also an advantage where one polypeptide expresses inherent pathogen-resistance but is somewhat labile.

Exemplary polynucleotides encoding proteins that confer increased pathogen resistance that may be stacked with polynucleotides of the invention include polynucleotides encoding proteins that confer increased ASR resistance as described in US Patent publication Nos. US 20200354739 and PCT Publications Nos. WO2019103918, WO2021154632A1, WO2021022022, WO2021022026, WO2021022101, WO2021260673, and WO2021263249, each of which is incorporated by reference in its entirety.

Exemplary polynucleotides that may be stacked with polynucleotides of the invention encoding a novel R-gene include polynucleotides encoding polypeptides conferring resistance to pests/pathogens such as viruses, nematodes, insects or fungi, and the like. Exemplary polynucleotides that may be stacked with polynucleotides of the invention include polynucleotides encoding: polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like; traits desirable for disease or herbicide resistance (e.g., fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); a gene encoding an aryloxyalkanoate dioxygenase conferring resistance to certain classes of auxin and acetylCoA carboxylase herbicides (e.g. in PCT Publication Nos. WO 2008/141154, WO 2007/053482 or a tfdA gene giving resistance to 2,4 D in U.S. Pat. No. 6,153,401); a gene encoding a dicamba monoxygenase (Behrens et al. (2007) Science, 316, 1185) conferring resistance to dicamba; a gene encoding a homogentisate solanesyltransferase (HST) conferring resistance to HST-inhibiting herbicides (PCT Publication No. WO 2010/029311); a gene encoding a nitrilase conferring resistance to a nitrile-containing herbicide (e.g the bxnA bromoxynil nitrilase); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304:1151-1154; and in U.S. Patent Application Publication Nos. 20070004912, 20050246798, and 20050060767)); glufosinate resistance (e.g, phosphinothricin acetyl transferase genes PAT and BAR, described in U.S. Pat. Nos. 5,561,236 and 5,276,268); a cytochrome P450 or variant thereof that confers herbicide resistance or tolerance to, inter alia, HPPD herbicides (U.S. Patent Application Publication No. 20090011936; U.S. Pat. Nos. 6,380,465; 6,121,512; 5,349,127; 6,649,814; and 6,300,544; and PCT Publication No. WO 2007/000077); and traits desirable for processing or process products such as high oil (e.g., U.S. Pat. No. 6,232,529); modified oils (e.g., fatty acid desaturase genes (U.S. Pat. No. 5,952,544; PCT Publication No. WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)).

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, PCT Publication Nos. WO 99/25821, WO 99/25854, WO 99/25840, WO 99/25855, and WO 99/25853.

### Disease Resistant Soybean Plants and Germplasms

In the plants provided herein, the polynucleotides as described earlier this disclosure is a heterologous nucleic acid sequence in the genome of the plant. As used herein, the term “heterologous” in the context of a chromosomal segment refers to one or more DNA sequences (e.g., genetic loci) in a configuration in which they are not found in nature, for example as a result of a recombination event between homologous chromosomes during meiosis, or for example as a result of introduction of a transgenic sequence, or for example as a result of modification through gene editing.

Although soybean plants are used to exemplify the composition and methods throughout the application, a polynucleotide as provided herein may be introduced to any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Glycine (soybean or soya bean) is a genus in the bean family Fabaceae. The Glycine plants can be Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja Seib. Et Zucc., Glycine max (L.) Merrill., Glycine tabacina, or Glycine tomentella.

In some embodiments, the plants provided herein are elite plants or derived from an elite line.

As used herein, an “elite line” is an agronomically superior line that has resulted from many cycles of breeding and selection for superior agronomic performance. Numerous elite lines are available and known to those of skill in the art of soybean breeding. An “elite population,” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from, and/or can give rise to, a plant with superior agronomic performance, such as an existing or newly developed elite line of soybean.

An “elite” plant is any plant from an elite line, such that an elite plant is a representative plant from an elite variety. In some embodiments, the soybean plant comprising a polynucleotide encoding any one of the polypeptides disclosed herein is an elite soybean plant. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB 17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

A Disease resistant soybean plant or germplasm of the present invention may be produced by any method whereby an R-gene of the present invention is introduced into the soybean plant or germplasm, including, but not limited to, transformation, protoplast transformation or fusion, a double haploid technique, embryo rescue, gene editing, conventional breeding, and/or by any other nucleic acid transfer system.

In some embodiments, the soybean plant or germplasm comprises a non-naturally occurring variety of soybean. In some embodiments, the soybean plant or germplasm is at least about 50%, 55%, 60%, 65%, 70%, 75%, 80%, 85%, 90%, 95%, 96%, 97%, 98%, 99% or 100% identical to that of an elite variety of soybean.

The disease resistant soybean plant or germplasm may be the progeny of a cross between an elite variety of soybean and a variety of soybean that comprises an R-gene for enhanced Disease tolerance or resistance (e.g. ASR) wherein the R-gene is a novel gene encoding a protein that confers increased pathogen resistance; a R-gene that is substantially identical to any of SEQ ID NOs: 1, 2-4, 11-12; or a R-gene encoding a polypeptide that is substantially identical to SEQ ID NO: 5 while conferring or enhancing pathogen resistance (e.g., ASR resistance) in the plant. In many examples, alternative embodiments of the R-gene will have at least 90%, 91%, 92%, 93%, 94%, 95%, 96%, 97%, 98%, or 99% homology to any of SEQ ID NOs: 1, 2-4, and 11-12. In many examples, alternative embodiments of the R-gene will have at least 90%, 91%, 92%, 93%, 94%, 95%, 96%, 97%, 98%, or 99% homology to a nucleic acid molecule encoding the polypeptide of SEQ ID NO: 5, or a nucleic acid molecule encoding a polypeptide having at least 90% homology to SEQ ID NO: 5, while providing ASR resistance in the plant. In many examples, the polypeptide encoded by the R-gene will have at least 90%, at least 91%, at least 92%, at least 93%, at least 94%, at least 95%, at least 96%, at least 97%, at least 98%, or at least 99% homology to SEQ ID NO: 5.

In particular embodiments, the disease resistant soybean plant or germplasm may be the progeny of a cross between an elite variety of soybean and a variety of soybean that comprises an R-gene for enhanced Disease tolerance (e.g. ASR) wherein the R-gene is a novel gene encoding a protein conferring enhanced pathogen resistance, wherein the R-gene is substantially identical to any of SEQ ID NOs: 1, 2-4, 11-12, or a R-gene encoding a polypeptide that is substantially identical to SEQ ID NO: 5 while conferring ASR resistance in the plant. In many examples, alternative embodiments of the R-gene will have at least 90%, 91%, 92%, 93%, 94%, 95%, 96%, 97%, 98%, or 99% homology to any of SEQ ID NOs: 1, 2-4, 11-12. In many examples, alternative embodiments of the R-gene will have at least 90%, 91%, 92%, 93%, 94%, 95%, 96%, 97%, 98%, or 99% homology to a nucleotide sequence encoding the polypeptide of SEQ ID NO: 5. In many examples, the polypeptide encoded by the R-gene of SEQ ID NOs: 1, 2-4, and 11-12 will comprise one of at least 90%, at least 91%, at least 92%, at least 93%, at least 94%, at least 95%, at least 96%, at least 97%, at least 98%, or at least 99% homology to SEQ ID NO: 5 while conferring ASR resistance.

The disease resistant soybean plant or germplasm may be the progeny of an introgression wherein the recurrent parent is an elite variety of soybean and the donor comprises an R-gene associated with enhanced disease tolerance and/or resistance wherein the donor carries a R-gene having substantial identity to any of SEQ ID NOs: 1, 2-4, and 11-12 or a R-gene encoding a polypeptide having substantial identity to SEQ ID NO: 5. In some embodiments, the plant will comprise a R-gene having at least 90%, 91%, 92%, 93%, 94%, 95%, 96%, 97%, 98%, or 99% homology to any of SEQ ID NOs: 1, 2-4, 11-12 and increased tolerance to tolerance to ASR as compared to a plant not comprising the R-gene. In some embodiments, the plant will comprise a R-gene encoding a protein having at least 90%, 91%, 92%, 93%, 94%, 95%, 96%, 97%, 98%, or 99% homology to SEQ ID NO: 5 and increased tolerance to tolerance to ASR as compared to a plant not comprising the R-gene.

The disease resistant soybean plant or germplasm may be the progeny of a cross between a first elite variety of soybean (e.g., a tester line) and the progeny of a cross between a second elite variety of soybean (e.g., a recurrent parent) and a variety of soybean that comprises an R-gene.

A disease resistant soybean plant and germplasm of the present invention may comprise one or more R-genes of the present invention (e.g., any of SEQ ID NOs: 1, 2, 3, 4, 6, 7, 11 and 12).

In some embodiments, the plants provided herein can comprise one or more additional polynucleotides that encode an additional polypeptide that can confer a phenotype of increased pathogen resistance.

In specific embodiments, the plants, plant parts or seeds having the heterologous polynucleotide or polypeptide disclosed herein or active variants and fragment thereof can have a modified level of expression of the polynucleotide or polypeptide (i.e., an increase or a decrease in expression level). In other embodiments, the plants, plant parts or seeds having the heterologous polynucleotide or polypeptide disclosed herein or active variants and fragment thereof can have a modified level of activity of the polypeptide (i.e., an increase or a decrease in activity level). Methods to generate such modified levels of expression or activity are disclosed elsewhere herein and include, but are not limited to, breeding, gene editing, and transgenic techniques.

Plants produced as described above can be propagated to produce progeny plants, and the progeny plants that have stably incorporated into its genome a polynucleotide conferring increased pathogen resistance can be selected and can be further propagated if desired. The term “progeny,” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

In addition to the phenotypic traits, the genetic characteristic of the plant as represented by its genetic marker profile can be used to select plants of desired traits. The term “marker-based selection” refers to the use of genetic markers to detect one or more nucleic acids from the plant, where the nucleic acid is associated with a desired trait to identify plants that carry genes for desirable (or undesirable) traits. Markers include but are not limited to Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as Microsatellites, and Single Nucleotide Polymorphisms (SNPs). There are known sets of public markers that are being examined by ASTA and other industry groups for their applicability in standardizing determinations of what constitutes an essentially derived variety under the US Plant Variety Protection Act. However, these standard markers do not limit the type of marker and marker profile which can be employed in breeding or developing backcross conversions, or in distinguishing varieties or plant parts or plant cells or verify a progeny pedigree. Primers and PCR protocols for assaying these and other markers are disclosed in the Soybase (sponsored by the USDA Agricultural Research Service and Iowa State University) located at the world wide web at 129.186.26.94/SSR.html.

In one embodiment, the markers used to identify the plants comprising the polynucleotides disclosed herein are SNPs. Non-limiting examples of SNP genotyping methods include hybridization, primer extension, oligonucleotide ligation, nuclease cleavage, minisequencing and coded spheres. Such methods are well known and disclosed in e.g., Gut, I. G., Hum. Mutat. 17: 475-492 (2001); Shi, Clin. Chem. 47(2): 164-172 (2001); Kwok, Pharmacogenomics 1(1): 95-100 (2000); and Bhattramakki and Rafalski, Discovery and application of single nucleotide polymorphism markers in plants, in PLANT GENOTYPING: THE DNA FINGERPRINTING OF PLANTS, CABI Publishing, Wallingford (2001). A wide range of commercially available technologies utilize these and other methods to interrogate SNPs, including Masscode Sup™/Sup (Qiagen, Germantown, MD, (Hologic, Madison, WI), (Applied Biosystems, Foster City, CA), (Applied Biosystems, Foster City, CA) and Beadarrays Sup™/Sup (Illumina, San Diego, CA).

In some embodiments, an assay (e.g., generally a two-step allelic discrimination assay or similar), a KASP Sup™/Sup assay (generally a one-step allelic discrimination assay defined below or similar), or both can be employed to identify the SNPs that associate with increased pathogen resistance. In an exemplary two-step assay, a forward primer, a reverse primer, and two assay probes that recognize two different alleles at the SNP site (or hybridization oligos) are employed. The forward and reverse primers are employed to amplify genetic loci that comprise SNPs that are associated with increased pathogen resistance. The particular nucleotides that are present at the SNP positions are then assayed using the probes. In some embodiments, the assay probes and the reaction conditions are designed such that an assay probe will only hybridize to the reverse complement of a 100% perfectly matched sequence, thereby permitting identification of which allele (s) that are present based upon detection of hybridizations. In some embodiments, the probes are differentially labeled with, for example, fluorophores to permit distinguishing between the two assay probes in a single reaction. Exemplary methods of amplifying include employing a polymerase chain reaction (PCR) or ligase chain reaction (LCR) using a nucleic acid isolated from a soybean plant or germplasm as a template in the PCR or LCR.

In some embodiments, a number of SNP alleles together within a sequence, or across linked sequences, can be used to describe a haplotype for any particular genotype. Ching et al., BMC Genet. 3: 19 (2002) (14 pages); Gupta et al., (2001) Curr Sci. 80:524-535, Rafalski, Plant Sci. 162: 329-333 (2002). In some cases, haplotypes can be more informative than single SNPs and can be more descriptive of any particular genotype. For example, a single SNP may be allele “T” for a specific disease resistant line or variety, but the allele “T” might also occur in the soybean breeding population being utilized for recurrent parents. In this case, a combination of alleles at linked SNPs may be more informative. Once a unique haplotype has been assigned to a donor chromosomal region, that haplotype can be used in that population or any subset thereof to determine whether an individual has a particular gene. The use of automated high throughput marker detection platforms known to those of ordinary skill in the art makes this process highly efficient and effective.

These SNP markers can be used in a marker assisted breeding program to move traits, such as native traits or traits conferred by transgenes or traits conferred by genome editing, into the a desired plant background. As used herein, the term “native trait” refers to a trait already existing in germplasm, including wild relatives of crop species, or that can be produced by recombination of existing traits. For example, progeny plants from a cross between a donor soybean plant comprising in its genome a nucleic acid sequence encoding SEQ ID NO: 1, 2-4, 11 or 12, and a recipient soybean plant not comprising said nucleic acid sequence can be screened to detect the presence of the markers associated with increased pathogen resistance profile. Plants comprising said markers can be selected and verified for increased pathogen resistance as compared to control plants. In embodiments of the invention, plants comprising in their genome a nucleic acid sequence encoding SEQ ID NO: 1, 2-4, 11 or 12 can be identified, detected, or selected using any combination of the “favorable” SNP markers of Table 1 and/or 2.

Also provided herein are the kits and primers that can be used to introduce a polynucleotide sequence as described in this disclosure into a recipient plant or to detect a polynucleotide sequence as described in this disclosure in a plant.

In some embodiments, the kit may also comprise one or more probes having a sequence corresponding to or complementary to a sequence having 80% to 100% sequence identity with a specific region of the transgenic event or gene editing event. In some embodiments, the kit may comprise any reagent and material required to perform the assay or detection method.

In embodiments, molecular marker-based assays can be used to select parent lines for propagation and also to select progeny plants. In example embodiments, such marker-based assays may use any of the SNP markers of Table 1 and/or 2 to identify a plant with a “favorable” allele associated with the increased pathogen resistance trait. In embodiments, primer-based assays may be used to detect for the presence of an amplicon comprising the novel R-gene of the present invention. In example embodiments, such primer-based assays may use the primer pair of SEQ ID NOS: 8-9 or any of the primer pairs listed in Table 6. Further, a probe may be used to detect for the presence of the amplicon, such as using any probe listed in Table 6 or the probe of SEQ ID NO: 10.

In some embodiments, a plant cell, seed, or plant part or harvest product can be obtained from the plant produced as above and the plant cell, seed, or plant part can be screened using methods disclosed above for the evidence of stable incorporation of the polynucleotide. The term “stable incorporation” refers to the integration of a nucleic acid sequence into the genome of a plant and said nucleic acid sequence is capable of being inherited by the progeny thereof. As used herein, the term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

In some embodiments, plant products can be harvested from the plant disclosed above and processed to produce processed products, such as flour, soy meal, oil, starch, and the like. These processed products are also within the scope of this invention provided that they comprise a polynucleotide or polypeptide or variant thereof disclosed herein. Other soybean plant products include but are not limited to protein concentrate, protein isolate, soybean hulls, meal, flower, oil and the whole soybean itself.

### Disease Resistant Soybean Seeds

The present invention provides disease resistant soybean seeds. As discussed above, the methods of the present invention may be utilized to identify, produce and/or select a disease resistant soybean seed. In addition to the methods described above, a disease resistant soybean seed may be produced by any method whereby an R-gene is introduced into the soybean seed, including, but not limited to, transformation, protoplast transformation or fusion, a double haploid technique, embryo rescue, genetic editing (e.g. CRISPR or TALEN or MegaNucleases) and/or by any other nucleic acid transfer system.

In some embodiments, the disease resistant soybean seed comprises a non-naturally occurring variety of soybean. In some embodiments, the soybean seed is at least about 50%, 55%, 60%, 65%, 70%, 75%, 80%, 85%, 90%, 95%, 96%, 97%, 98% or 99% identical to that of an elite variety of soybean.

The disease resistant soybean seed may be produced by a disease resistant soybean plant identified, produced or selected by the methods of the present invention.

A disease resistant soybean seed of the present invention may comprise, be selected by or produced by use of one or more novel R-genes of the present invention.

### Methods for Producing a Plant Variety that has Increased Pathogen Resistance

Provided herein are methods of producing a plant that has increased pathogen resistance by introducing a nucleic acid sequence encoding a polypeptide as provided herein. A nucleic acid sequence may be introduced to a plant cell by various ways, for example, by transformation, by genome modification techniques (such as by genome editing), or by breeding. In one aspect, the plant can be produced by transforming the nucleic acid sequence encoding a polypeptide disclosed above into a recipient plant. In one aspect, the method can comprise editing the genome of the recipient plant so that the resulting plant comprises a polynucleotide encoding a polypeptide disclosed above. In yet another aspect, the method can comprise increasing the expression level and/or activity of the above-mentioned proteins in a recipient plant, for example, by enhancing promoter activity or replacing the endogenous promoter with a stronger promoter. In another aspect, the method can comprise breeding a donor plant comprising a polynucleotide as described above with a recipient plant and selecting for incorporation of the polynucleotide into the recipient plant genome.

In some embodiments, the method comprises transforming a polynucleotide disclosed herein or an active variant or fragment thereof into a recipient plant to obtain a transgenic plant, and said transgenic plant has increased pathogen resistance. Expression cassettes comprising polynucleotides encoding the polypeptides as described above can be used to transform plants of interest.

As used herein, the term “transgenic” and grammatical variations thereof refer to a plant, including any part derived from the plant, such as a cell, tissue or organ, in which a heterologous nucleic acid is integrated into the genome. In specific embodiments, the heterologous nucleic acid is a recombinant construct, vector or expression cassette comprising one or more nucleic acids. In other embodiments, a transgenic plant is produced by a genetic engineering method, such as Agrobacterium transformation. Through gene technology, the heterologous nucleic acid is stably integrated into chromosomes, so that the next generation can also be transgenic. As used herein, “transgenic” and grammatical variations thereof also encompass biological treatments, which include plant hybridization and/or natural recombination.

Transformation results in a transformed plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation typically involve introducing a nucleotide construct into a plant. In some embodiments, the transformation method is an Agrobacterium-mediated transformation. In some embodiments, the transformation method is a biolistic-mediated transformation. Transformation may also be performed by infection, transfection, microinjection, electroporation, microprojection, biolistics or particle bombardment, electroporation, silica/carbon fibers, ultrasound mediated, PEG mediated, calcium phosphate co-precipitation, poly cation DMSO technique, DEAE dextran procedure, Agrobacterium and viral mediated (e.g., Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; and 7,541,517; each of which is herein incorporated by reference. See, also, Rakoczy-Trojanowska, M. (2002) Cell Mol Biol Lett. 7:849-858; Jones et al. (2005) Plant Methods, Vol. 1, Article 5; Rivera et al. (2012) Physics of Life Reviews 9:308-345; Bartlett et al. (2008) Plant Methods 4: 1-12; Bates, G. W. (1999) Methods in Molecular Biology 111:359-366; Binns and Thomashow (1988) Annual Reviews in Microbiology 42:57 Sup′/Sup5-606; Christou, P. (1992) The Plant Journal 2:275-281; Christou, P. (1995) Euphytica 85: 13-27; Tzfira et al. (2004) TRENDS in Genetics 20:375-383; Yao et al. (2006) Journal of Experimental Botany 57:3737-3746; Zupan and Zambryski (1995) Plant Physiology 107:

Methods for transformation of chloroplasts are known in the art. See, for example, Svab et al. (1990) Proc. Natl. Acad. Sci. USA 87(21):8526-8530; Svab and Maliga (1993) Proc. Natl. Acad. Sci. USA 90(3):913-917; Staub and Maliga (1993) EMBO J. 12(2):601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91(15):7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

In some embodiments, the method comprises crossing a donor plant comprising a polynucleotide encoding a polypeptide disclosed herein with a recipient plant, and the polypeptide is able to confer increased pathogen resistance in the recipient plant. As used herein, the terms “crossing” and “breeding” refer to the fusion of gametes to produce progeny (e.g., by fertilization, such as to produce seed by pollination in plants). In some embodiments, a “cross,” “breeding,” or “cross-fertilization” is fertilization of one individual by another (e.g., cross-pollination in plants). The plant disclosed herein may be a whole plant, or may be a plant cell, seed, or tissue, or a plant part such as leaf, stem, pollen, or cell that can be cultivated into a whole plant.

In some embodiments, a progeny plant created by the crossing or breeding process is repeatedly crossed back to one of its parents through a process referred to herein as “backcrossing”. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in Techniques et Utilisations des Marqueurs Moleculaires Les Colloques, Vol. 72, pp. 45-56 (1995); and Openshaw et al., Marker-assisted Selection in Backcross Breeding, in Proceedings of the Symposium “Analysis of Molecular Marker Data,” pp. 41-43 (1994). The initial cross gives rise to the F1 generation. The term “BC1” refers to the second use of the recurrent parent, “BC2” refers to the third use of the recurrent parent, and so on.

In some embodiments, the donor soybean plant is a Glycine max plant. In some embodiments, the donor soybean plant is a Glycine soja plant. In some embodiments, the recipient soybean plant is an elite Glycine max plant or an elite Glycine soja plant.

In some embodiments, the polynucleotide sequences provided herein can be targeted to specific sites within the genome of a recipient plant cell. Such methods include, but are not limited to, meganucleases designed against the plant genomic sequence of interest CRISPR-Cas9, TALENs, and other technologies for precise editing of genomes (Feng, et al. Cell Research 23: 1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701: 147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol: 51-65); prime editing and transposases (Anzalone, A. et al., Nat Biotechnol. 2020 July; 38(7):824-844); translocation; and inversion.

Various embodiments of the methods described herein use gene editing. In some embodiments, gene editing is used to mutagenize the genome of a plant to produce plants having one or more of the polypeptides that is able to confer increased pathogen resistance.

In some embodiments, provided herein are plants transformed with and expressing gene-editing machinery as described above, which, when crossed with a target plant, result in gene editing in the target plant. In general, gene editing may involve transient, inducible, or constitutive expression of the gene editing components or systems. Gene editing may involve genomic integration or episomal presence of the gene editing components or systems.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., increased pathogen resistance, increased ASR resistance, etc. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest, e.g., SEQ ID NO: 2-4, 11, 12 or a polynucleotide encoding a polypeptide having the sequence of SEQ ID NO: 5 may be provided on/as a template. By designing the system such that one or more single strand or double strand breaks are introduced within, upstream, and/or downstream of the corresponding region in the genome of a plant not comprising the gene sequence of interest, this region can be replaced with the template. In some embodiments, the polynucleotide of interest is operably linked to a promoter and the expression of the polynucleotide of interest controlled by the promoter conferred increased pathogen resistance to the plant. In some embodiments, the promoter is a native promoter or an active variant or fragment thereof as described above. In some embodiments, the native promoter comprises SEQ ID NO: 15.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. Nos. 10,519,456 and 10,285,348 82, the entire content of which is herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In another aspect, provided herein is a method of editing plant genomic DNA. In some embodiments, the method comprises using a first soybean plant expressing a DNA modification enzyme and at least one optional guide nucleic acid as described above to pollinate a target plant comprising genomic DNA to be edited.

The various polynucleotides and variants thereof provided herein can be stacked with one or more polynucleotides encoding a desirable trait such as a polynucleotide that confers, for example, insect, disease or herbicide resistance or other desirable agronomic traits of interest including, but not limited to, traits associated with high oil content; high protein content; increased digestibility; balanced amino acid content; and high energy content. Such traits may refer to properties of both seed and non-seed plant tissues, or to food or feed prepared from plants or seeds having such traits.

As used herein, gene or trait “stacking” is combining desired genes or traits into one transgenic plant line. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. Another way to stack genes is by re-transforming a transgenic plant with another gene of interest. For example, gene stacking can be used to combine two different insect resistance traits, an insect resistance trait and a disease resistance trait, or a herbicide resistance trait (such as, for example, Btl1). The use of a selectable marker in addition to a gene of interest would also be considered gene stacking.

In some embodiments, a nucleic acid molecule or vector of the disclosure can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Polynucleotides conferring resistance/tolerance to an herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268, and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, modified oil profile, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g., E.C. 3.1.1.74).

Double stranded RNA molecules useful with the disclosure include but are not limited to those that suppress target insect genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

Non-limiting embodiments of the invention include proteins and nucleic acids that confer increased pathogen resistance when expressed. In embodiments, a polypeptide is selected from: (a) a polypeptide having the amino acid sequence shown in SEQ ID NO: 5, or any portion thereof, and having a heterologous amino acid sequence attached thereto, wherein expression of the polypeptide or portion thereof confers increased pathogen resistance on a plant; (b) a polypeptide comprising the amino acid sequence of SEQ ID NO: 5, and having substitution and/or deletion and/or addition of one or more amino acid residues, wherein expression of the polypeptide confers increased pathogen resistance on the plant; (c) a polypeptide having more than 99%, 98%, 97%, 96%, 95%, 94%, 93%, 92%, 91%, 90%, 85%, or more than 80% sequence identity with the amino acid sequence of SEQ ID NO: 5, wherein the polypeptide when expressed in a plant confers increased pathogen resistance on the plant; or (d) a fusion polypeptide comprising the amino acid sequence of SEQ ID NO: 5, or the polypeptide as defined in any one of (a) to (c). In embodiments, a nucleic acid molecule comprises (a) a nucleotide sequence encoding a protein having an amino acid sequence sharing at least 90%, 91%, 92%, 93%, 94%, 95%, 96%, 97%, 98%, 99%, or 100% sequence identity to SEQ ID NO: 5, wherein said nucleotide sequence comprises a heterologous nucleic acid sequence attached thereto and expression of the nucleic acid molecule confers increased pathogen resistance on the plant; (b) a nucleotide sequence encoding the aforementioned polypeptide; (c) the nucleotide sequence of part (a) comprising a sequence of any one of SEQ ID NOS: 2-4 and 11-12; or (d) the nucleotide sequence of part (a) having at least more than more than 99%, 98%, 97%, 96%, 95%, 94%, 93%, 92%, 91%, 90%, 85%, or more than 80% sequence identity to any one of SEQ ID NOs: 2-4 and 11-12.

Non-limiting embodiments of the invention include expression cassettes, vectors and DNA constructs comprising the aforementioned nucleic acid molecules and/or expressing the aforementioned polypeptides that confer increased pathogen resistance. In embodiments, an expression cassette comprises the aforementioned nucleic acid molecule of the invention or encodes the aforementioned polypeptide of the invention. In embodiments of the expression cassette, the nucleic acid molecule is operably linked to a promoter capable of directing expression in a plant cell. In some embodiments, the promoter is an endogenous promoter. In other embodiments, the promoter is an exogenous promoter. In particular embodiments, the promoter comprises any one of SEQ ID NOS: 13-15. In embodiments, a vector comprises the aforementioned nucleic acid molecule or the expression cassette. In embodiments, a transgenic cell comprises the nucleic acid molecule or the expression cassette of the invention.

Non-limiting embodiments include transgenic plants that have increased pathogen resistance. In embodiments, a plant is provided having stably incorporated into its genome a nucleic acid sequence operably linked to a promoter active in the plant, wherein the nucleic acid sequence encodes a polypeptide having: an amino acid sequence that has at least 85%, 90%, 91%, 92%, 93%, 94%, 95%, 96%, 97%, 98%, or 99% sequence identity to SEQID NO: 5; or an amino acid sequence set forth in SEQ ID NO: 5, wherein said nucleic acid sequence is heterologous to the plant, and wherein the plant has increased pathogen resistance as compared to a control plant not comprising the nucleic acid sequence. In embodiments of the plant, the nucleic acid sequence comprises at least 85% identity, at least 90% identity, or at least 95% identity to any one of SEQ ID NOs: 2-4 and 11-12; or the nucleic acid sequence is SEQ ID NO: 2, 3, 4, 11 or 12. In embodiments of the plant, the nucleic acid sequence is introduced into the genome by transgenic expression. In embodiments of the plant, the promoter is an endogenous promoter. In particular embodiments, the endogenous promoter comprises at least 90%, 95%, 96%, 97%, 98%, 99%, or 100% sequence identity to SEQ ID NO: 15. In embodiments of the plant, the promoter is a heterologous promoter comprising at least 90%, 95%, 96%, 97%, 98%, 99%, or 100% sequence identity to SEQ ID NO: 13 or 14. In embodiments of the plant, the promoter is a constitutive promoter, inducible promoter, or a tissue-specific promoter. In particular embodiments, the plant is a dicot plant, such as soybean plant or an elite soybean plant. In other embodiments, the plant is a monocot plant, such as a monocot plant is selected from the group consisting of rice, wheat, maize, and sugar cane. In embodiments, the plant is an agronomically elite plant having a commercially significant yield and/or commercially susceptible vigor, seed set, standability, threshability, abiotic/biotic resistance, or herbicide tolerance. In any of the aforementioned plant embodiments, the plant has increased resistance to any one of the following pathogens: soy cyst nematode, bacterial pustule, root knot nematode, frog eye leaf spot, phytophthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme. In particular embodiments, the plant is a soybean plant that has increased resistance to ASR as compared to the control plant.

Non-limiting embodiments of the invention further include gene edited plants with increased pathogen resistance. Embodiments of gene edited plants include a plant, the genome of which has been edited to comprise a nucleic acid sequence encoding at least one polypeptide having at least 80%, 85%, 90%, 91%, 92%, 93%, 94%, 95%, 96%, 97%, 98%, 99%, or 100% to SEQ ID NO: 5, wherein said polypeptide confers increased pathogen resistance relative to a control plant, wherein the plant does not comprise said nucleic acid sequence before the genome editing. In embodiments of the plant, the nucleic acid sequence is introduced into said plant genome by genome editing of the nucleic acid sequence set forth in any one of SEQ ID NOS: 1, 2, 3 4, 11 and/or 12. In embodiments, the genome editing comprises duplication, inversion, promoter modification, terminator modification and/or splicing modification of the nucleic acid sequence. In particular embodiments, the genome editing is accomplished through CRISPR, TALEN, meganucleases, or through modification of genomic nucleic acids. In embodiments, the gene edited plant is an agronomically elite plant having a commercially significant yield and/or commercially susceptible vigor, seed set, standability, threshability, abiotic/biotic resistance, or herbicide tolerance. In embodiments, the nucleic acid sequence is operably linked to a heterologous promoter, and wherein the heterologous promoter is active in the plant. In particular embodiments, the heterologous promoter active in the plant has at least 95% sequence identity to one of SEQ ID NOS: 13 and 14. In other embodiments, the heterologous promoter is a native promoter or active variant or fragment thereof, and wherein optionally the native promoter has at least 90%, 91%, 92%, 93%, 94%, 95%, 96%, 97%, 98%, 99%, or 100% sequence identity to SEQ ID NO: 15. In particular embodiments, the plant is a soybean plant having increased resistance to Asian Soy Rust relative to the control plant.

Non-limiting embodiments further include Glycine max plants with increased pathogen resistance. In embodiments, an elite Glycine max plant is provided having in its genome a nucleic acid sequence from a donor Glycine plant, wherein the donor Glycine plant is a different strain from the elite Glycine max plant, and wherein the nucleic acid sequence encodes at least one polypeptide having at least 90%, 91%, 92%, 93%, 94%, 95%, 96%, 97%, 98%, 99%, or 100% sequence identity to SEQ ID NO: 5, wherein said polypeptide confers increased pathogen resistance on the elite Glycine max plant as compared to a control plant not comprising said nucleic acid sequence. In embodiments, the donor Glycine plant is a Glycine tomentella plant, or a progeny thereof. In particular embodiments, the Glycine tomentella plant is a plant of Glycine tomentella accession line PI505267 or a progeny thereof. In further embodiments, the nucleic acid sequence has at least 90%, 91%, 92%, 93%, 94%, 95%, 96%, 97%, 98%, 99%, or 100% sequence identity to any one of SEQ ID NOs: 2-4 and 11-12. In still other embodiments, the nucleic acid sequence has at least 90%, 91%, 92%, 93%, 94%, 95%, 96%, 97%, 98%, 99%, or 100% identity to SEQ ID NO: 1, or a functional fragment thereof, wherein the functional fragment comprises at least at least 10%; at least 15%; at least 20%; at least 25%; at least 30%; at least 35%; at least 40%; at least 45%; at least 50%; at least 55 at least 60%; at least 65%; at least 70%; at least 75%; at least 80%; at least 85%; at least 90%; at least 95%; 96%, 97%, 98%, or 99% of SEQ ID NO: 1 and confers increased pathogen resistance. In embodiments, the nucleic acid sequence comprises a SNP marker associated with increased ASR resistance, wherein said SNP marker is any of the favorable markers of Table 1 and/or 2. In particular embodiments, the nucleic acid sequence from the donor glycine plant is inserted into chromosome 3 of the plant. In embodiments, said nucleic acid sequence is introduced into said plant genome by genome editing of genomic sequences corresponding to and comprising any one of SEQ ID NOs: 1, 2-4, and 11-12, wherein the genome editing confers the elite plant with enhanced resistance to the pathogen, and wherein the gene editing is by CRISPR, TALEN, meganucleases, or through modification of genomic nucleic acids. In other embodiments, said nucleic acid sequence is introduced into said plant genome by transgenic expression of: (a) a nucleic acid sequence having at least 90%, 91%, 92%, 93%, 94%, 95%, 96%, 97%, 98%, 99%, or 100% identity to any one of SEQ ID NOS: 2-4 and 11-12, (b) a nucleic acid sequence encoding a polypeptide having at least 90%, 91%, 92%, 93%, 94%, 95%, 96%, 97%, 98%, 99%, or 100% identity to SEQ ID NO: 5; or (c) a nucleic acid sequence encoding a polypeptide having the sequence of SEQ ID NO: 5; wherein said polypeptide confers enhanced pathogen resistance on the elite Glycine max plant. In particular embodiments, said nucleic acid sequence is introgressed into the genome of said plant through the use of one or more of: (a) chemically induced chromosome doubling; and (b) doubling of an elite Glycine max line to obtain a doubled Glycine max plant before crossing the doubled plant with a Glycine tomentella plant derived from accession line PI505267 or a progeny thereof, as described in Example 3, the Glycine tomentella plant comprising said nucleic acid sequence. In embodiments, the plant has increased resistance to any one or more of the following pathogens: soy cyst nematode, bacterial pustule, root knot nematode, frog eye leaf spot, phytophthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme. In particular embodiments, the plant has increased resistance to Asian Soybean Rust. In embodiments, elite Glycine max plant is an agronomically elite Glycine max plant having a commercially significant yield and/or commercially susceptible vigor, seed set, standability, threshability, abiotic/biotic resistance, or herbicide tolerance.

Non-limiting embodiments of the invention include plants, plant parts and products with increased pathogen resistance. In embodiments, a progeny plant from any of the aforementioned plants is provided, wherein the progeny plant has stably incorporated into its genome the nucleic acid sequence of the invention. In embodiment, a plant cell, seed, or plant part is provided that is derived from any of the aforementioned plants, wherein said plant cell, seed or plant part has stably incorporated into its genome the nucleic acid sequence.

Non-limiting embodiments of the invention include methods of producing transgenic plants. In embodiments, use of the aforementioned polypeptide or nucleic acid molecule or expression cassette or vector or transgenic cell of the invention is provided in conferring increased resistance to Asian Soy Rust (ASR). In particular embodiments, the method comprises use of the expression cassette of the invention in a cell, wherein the expression level and/or activity of the polypeptide in the cell is increased, and the resistance of the cell to Asian Soy Rust is enhanced. Embodiments include a method for improving the resistance of a plant against ASR, comprising increasing the expression level and/or activity of the polypeptide of the invention in the plant. In embodiments, the increasing comprises increasing the expression level and/or activity of the nucleic acid molecule of the invention in the plant. In embodiments, the increasing the expression level and/or activity in the plant is realized by transgenic means or by breeding. Embodiments are provided for a method for producing a transgenic plant with improved resistance against ASR, comprising: introducing the nucleic acid molecule or the expression cassette of the invention to a recipient plant to obtain a transgenic plant, wherein the transgenic plant has increased resistance against ASR compared to the recipient plant.

Non-limiting embodiments include methods of producing plants with increased pathogen resistance including by breeding methods. In embodiments, a method of producing a soybean plant having increased pathogen resistance comprises the steps of: a) providing a donor soybean plant comprising in its genome a nucleic acid sequence encoding at least one polypeptide having at least 90% identity or 95% identity to SEQ ID NO: 5, wherein said nucleic acid sequence confers to said donor soybean plant increased pathogen resistance as compared to another donor soybean plant not comprising said nucleic acid sequence in its genome; b) crossing the donor soybean plant of a) with a recipient soybean plant not comprising said nucleic acid sequence; and c) selecting a progeny plant from the cross of b) by detecting the presence of the nucleic acid sequence, or the presence of one or more molecular markers associated with the nucleic acid sequence in the progeny plant, thereby producing a soybean plant having increased pathogen resistance. In embodiments of the method, the molecular marker is a single nucleotide polymorphism (SNP), a quantitative trait locus (QTL), an amplified fragment length polymorphism (AFLP), randomly amplified polymorphic DNA (RAPD), a restriction fragment length polymorphism (RFLP) or a microsatellite. In particular embodiments, the molecular marker is at least one favorable SNP marker selected from Table 1 and/or Table 2, or a molecular marker located within 20 cM, 10 cM, 5 cM, 1 cM, or 0.5 cM of a favorable SNP marker selected from Table 1 or Table 2. In embodiments, one or more of the donor soybean plant and the recipient soybean plant is an elite Glycine max plant. Embodiments include a method for producing a Glycine max plant having increased resistance to ASR, the method comprising the steps of: providing a Glycine tomentella plant line, or progeny thereof comprising a nucleic acid sequence encoding at least one polypeptide having at least 90%, 91%, 92%, 93%, 94%, 95%, 96%, 97%, 98%, 99%, or 100% identity to SEQ ID NO: 5; carrying out the embryo rescue method essentially as described in U.S. Pat. No. 7,842,850 or transgenically; collecting the seeds resulting from the method of b); and regenerating the seeds of c) into plants. In particular embodiments, the Glycine tomentella plant line is accession line PI505267, or a progeny thereof. In embodiments, the nucleic acid sequence is: a nucleic acid sequence comprising at least 85%, 90%, 91%, 92%, 93%, 94%, 95%, 96%, 97%, 98%, 99%, or 100% identity to any one of SEQ ID NOs: 2-4 and 11-12; or the nucleic acid sequence of SEQ ID NO: 2, 3, 4, 11 or 12;

Embodiments include a method of producing a Glycine max plant with increased resistance to Asian Soy Rust (ASR), the method comprising the steps of: a) isolating a nucleic acid from a Glycine max plant; b) detecting in the nucleic acid of a) at least one molecular marker associated with a nucleic acid sequence comprising any one of SEQ ID NO: 2-4, wherein said nucleic acid sequence confers to the Glycine max plant increased ASR resistance; c) selecting a Glycine max plant based on the presence of the molecular marker detected in b); and d) producing a Glycine max progeny plant from the plant of c) identified as having said molecular marker associated with increased ASR resistance. In embodiments, the molecular marker is a favorable SNP marker selected from Table 1 or Table 2, or a molecular marker located within 20 cM, 10 cM, 5 cM, 1 cM, or 0.5 cM of a favorable SNP marker selected from Table 1 and/or Table 2. In embodiments, the detecting comprises amplifying a molecular marker locus or a portion of the molecular marker locus and detecting the resulting amplified molecular marker amplicon. In embodiments, the amplifying comprises employing a polymerase chain reaction (PCR) or ligase chain reaction (LCR) using a nucleic acid isolated from a soybean plant or germplasm as a template in the PCR or LCR. In particular embodiments, the amplifying further comprises employing a primer pair selected from the group comprising: the primer pair of SEQ ID NOS: 8-9; and a primer pair from the primers of Table 6. In particular embodiments, the detecting further comprises employing a nucleic acid probe selected from the group comprising: the probe of SEQ ID NO: 10 and a probe from the probes of Table 6. In embodiments, the nucleic acid is DNA or RNA. In embodiments, a plant is provided produced by any of the aforementioned methods.

Non-limiting embodiments include a method of conferring increased ASR resistance to a plant, comprising: a) introducing into the genome of the plant a nucleic acid molecule operably linked to a promoter active in the plant, wherein the nucleic acid sequence is stably incorporated into the genome, wherein the nucleic acid sequence encodes a polypeptide having (i) an amino acid sequence comprising at least 85%, 90%, 91%, 92%, 93%, 94%, 95%, 96%, 97%, 98%, or 99% identity to SEQ ID NO: 5, or (ii) an amino acid sequence as set forth in SEQ ID NO: 5, wherein said nucleic acid sequence is heterologous to the plant, and wherein expression of said nucleic acid sequence increases ASR resistance compared to a control plant not expressing said nucleic acid sequence. In embodiments, the nucleic acid sequence is introduced into the genome of the plant by transformation. In other embodiments, the nucleic acid sequence is introduced into the genome of the plant by crossing a donor plant comprising the nucleic acid sequence with the plant to produce a progeny plant having increased ASR resistance. In particular embodiments, the nucleic acid sequence is inserted into chromosome 3. In particular embodiments, the promoter is an exogenous promoter, and wherein optionally the exogenous promoter comprises SEQ ID NO: 13 or 14. In other embodiments, the promoter is an endogenous promoter, and wherein optionally the endogenous promoter comprises SEQ ID NO: 15. In embodiments, the method further comprises screening for the introduced nucleic acid sequence with PCR and/or sequencing. In particular embodiments, the plant is a dicot plant, and wherein the dicot plant is a soybean plant. In other embodiments, the plant is a monocot plant selected from the group consisting of rice, wheat, maize, and sugar cane. In embodiments, a plant is produced by any of the aforementioned methods.

In non-limiting embodiments, a primer pair is provided for amplifying the nucleic acid molecule of the invention. In particular embodiments, the primer pair is the primer pair of SEQ ID NOS: 8-9 or a primer pair selected from the primers of Table 6. In embodiments, a primer diagnostic for ASR resistance is provided, wherein said primer can be used in a PCR reaction to indicate the presence of an allele associated with ASR resistance, wherein said allele is any favorable allele as described in Table 1 and/or Table 2 and wherein said primer is any primer selected from the primers of Table 6.

### EXAMPLES

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1: Identification of an ASR Resistant Wild Glycine Line

Wild glycine lines were evaluated for ASR resistance against sixteen rust strains collected across a diverse range of environments. The rust data were generated using single pustule derived isolates from USDA-ARS (FL Q09, FL Q12, LABR13, FLQ11) and field populations (FL Q15, NC06, Vero, GLC15, UBL, BR south and BR central). The screening was carried out in contained facilities. Each wild glycine line was evaluated over a multiple day course of infection and rated at various time points. The rating and evaluation were performed using methods well known in the art, based upon Burdon and Speer (Euphytica, 33: 891-896, 1984; also TAG, 1984). Each accession line of interest was screened >2 times with ˜4 plants each time in North & South America using the large diverse panel of rust isolates. Based on the analysis of the rust data, wild Glycine tomentella accession line PI505267 was determined to be an ASR resistant wild glycine line of interest.

### Example 2: Allele Mining & Associations to PI505267 ASR Loci

Chromosome discovery for causal loci in the tetraploid soybean population, PI505267, was carried out using Data2Bio's Genomic Bulked Segregant Analysis (gBSA) technology (Ames, IA). Data2Bio generated several libraries from DNA samples extracted from two susceptible tissue pools and one resistant tissue pool and sequenced these in eight (8) Illumina HiSeq2000 2×100 bp Paired-End (PE) lanes (San Diego, CA). Processing of raw data including quality trimming, alignment, SNP discovery and SNP impact was performed. After various filtering steps, a plurality of informative SNPs were identified in the PI505267 genome that significantly associated with ASR resistance. A Bayesian approach was then used to calculate trait-associated probabilities. Next, a physical map of trait-associated SNPs (probability cutoff 0.01) for the top contigs were created. Clustering of the SNPs indicated that the ASR resistance loci is located on or near one particular scaffold, Contig 0133 (SEQ ID NO: 1), which was identified and mapped to chromosome 3 of Glycine tomentella accession line PI505267. The context sequences associated with the SNPs from this scaffold were aligned to the public G. max genome to create a chromosome-level understanding of the mapping interval. Data indicated that the causative R-gene(s) may map within or near the interval from 9.28 to 16.48 MB of Contig 0133 on chromosome 3. Genes from this interval were expected to encode polypeptide(s) that may be transgenically expressed or genetically modified (i.e., gene edited via TALEN or CRISPR) in plants to confer disease resistance (e.g., Asian Soy Rust (ASR) resistance).

A listing of single nucleotide polymorphisms (SNPs) within SEQ ID NO: 1 that associate with ASR resistance is provided below at Tables 1 and 2. SNPs were identified and verified by crossing the ASR resistant line PI505267 with two different susceptible female lines. All alleles for the identified SNPs were determined to be significantly linked with resistance or susceptibility (p<0.05). Detection of the presence of a molecular marker, such as any of the favorable markers of Tables 1 and/or 2, in the nucleic acid isolated from a plant, can be used to identify or select a plant as having the ASR resistance allele derived from the ASR resistant Glycine tomentella line, such as due to the introgression of the chromosomal interval of SEQ ID NO: 1, or a functional fragment thereof (such as functional fragment of the chromosomal interval of SEQ ID NO: 1 comprising the causative R-gene).

TABLE 1
List of SNPs derived from crossing of Resistant male with susceptible female 1. SNPs listed in format: (SNP ID,
SNP position relative to reference genome, Favorable allele, Unfavorable allele), each separated by a space.

(1 9280996 A C); (2 9281013 G A); (3 9281195 C T); (4 9281200 A T); (5 9281201 C A); (6 9281223 C T); (7
9281276 A G); (8 9281485 C T); (9 9281647 G A); (10 9281724 AC A); (11 9281784 A C); (12 9281826 C
T); (13 9281835 G A); (14 9281849 G A); (15 9282235 C T); (16 9283196 G C); (17 9283409 A G); (18
9283754 T C); (19 9283852 T C); (20 9284242 A G); (21 9284284 T A); (22 9284396 C T); (23 9284517 A
T); (24 9284544 A T); (25 9284917 CT C); (26 9285271 A G); (27 9285329 T C); (28 9285641 C A); (29
9285701 A G); (30 9287126 G A); (31 9287346 T G); (32 9287696 T G); (33 9287730 G A); (34 9288083 T
A); (35 9288197 C T); (36 9288204 G GA); (37 9288360 T C); (38 9288370 G A); (39 9288405 G A); (40
9288427 T C); (41 9288663 G T); (42 9288686 T C); (43 9288747 G GTT); (44 9288939 T C); (45 9289360 A
ATTGTGGAAC); (46 9289409 A G); (47 9289584 T C); (48 9290113 A G); (49 9290269 A G); (50 9290321 A
T); (51 9290460 C T); (52 9290465 C T); (53 9290483 T C); (54 9290553 T C); (55 9290730 T G); (56
9291087 C T); (57 9291179 TG T); (58 9291229 G T); (59 9291251 G A); (60 9291357 A T); (61 9291583 C
G); (62 9291658 G A); (63 9291902 T C); (64 9291967 C T); (65 9292150 T C); (66 9292231
ATATGCTGAAAGTTTTT A); (67 9292503 C T); (68 9292772 G A); (69 9292792 G A); (70 9292832 C A); (71
9292848 G A); (72 9292864 A G); (73 9292865 G T); (74 9292866 C T); (75 9293079 G A); (76 9293132 C
T); (77 9293138 G A); (78 9293190 T G); (79 9293329 T C); (80 9293351 T A); (81 9293521 C T); (82
9293588 C T); (83 9293603 A C); (84 9293674 T C); (85 9293699 G A); (86 9293925 C T); (87 9294157 C
A); (88 9294234 A G); (89 9294289 T C); (90 9294612 G T); (91 9294776 C T); (92 9294966 GTA G); (93
9295411 G A); (94 9295472 C T); (95 9295596 T G); (96 9295618 A C); (97 9295751 T C); (98 9295937 A
T); (99 9296123 C CAA); (100 9296379 G A); (101 9296384 T C); (102 9296404 T A); (103 9296487 T
C); (104 9296728 T A); (105 9296787 T C); (106 9296822 T A); (107 9296823 A T); (108 9297264 C G); (109
9297354 A G); (110 9297425 C T); (111 9297796 T C); (112 9297798 T C); (113 9297884 C T); (114
9297889 C T); (115 9297939 T C); (116 9298007 T G); (117 9298047 G A); (118 9298211 T A); (119
9298394 T C); (120 9298626 C A); (121 9298708 A G); (122 9298754 G A); (123 9298845 C T); (124
9298846 T C); (125 9298946 A T); (126 9299120 G T); (127 9299177 C T); (128 9299209 T G); (129
9299247 G A); (130 9299327 C T); (131 9299776 C A); (132 9300267 G T); (133 9300355 A G); (134
9300405 G T); (135 9301458 A G); (136 9301541 CT C); (137 9301543 A G); (138 9301769 G A); (139
9301843 T C); (140 9301914 A AT); (141 9302189 G A); (142 9302192 A G); (143 9302333 T TG); (144
9302588 A G); (145 9302849 T C); (146 9302877 G C); (147 9302948 A G); (148 9302955 G C); (149
9303130 C G); (150 9303367 T C); (151 9303439 A G); (152 9303535 C T); (153 9303598 C T); (154
9303985 C CTACTCAA); (155 9304281 G A); (156 9305374 C T); (157 9305841 C T); (158 9305944 C
T); (159 9305950 C G); (160 9306100 A C); (161 9307000 C T); (162 9307076 A G); (163 9307127 T C); (164
9307261 G A); (165 9307291 A C); (166 9307379 C A); (167 9307667 G A); (168 9307730 T C); (169
9308097 G C); (170 9309199 A G); (171 9309420 T C); (172 9309498 C T); (173 9309860 T G); (174
9309950 AT A); (175 9310002 T C); (176 9310099 T C); (177 9310299 A T); (178 9311612 T A); (179
9311642 C T); (180 9311656 G A); (181 9311677 C T); (182 9311734 T C); (183 9311754 G A); (184
9311759 G A); (185 9311762 TG T); (186 9311835 C T); (187 9311873 G T); (188 9312082 G A); (189
9312273 G A); (190 9312427 T C); (191 9312529 G A); (192 9312575 T C); (193 9312591 G A); (194
9312701 T A); (195 9312872 G A); (196 9312944 C A); (197 9313018 T C); (198 9313213 C A); (199
9313398 C T); (200 9314094 C T); (201 9314218 C A); (202 9314770 A G); (203 9314884 T C); (204
9314924 G T); (205 9315263 A G); (206 9315836 T C); (207 9315999 T G); (208 9316000 A G); (209
9316145 A G); (210 9316189 A G); (211 9316195 C T); (212 9316203 A G); (213 9316326 A G); (214
9316493 C T); (215 9316655 A G); (216 9316656 A C); (217 9316695 CTA C); (218 9316703 A G); (219
9316769 G A); (220 9317531 C A); (221 9317571 G A); (222 9317954 T C); (223 9318210 G A); (224
9318370 T C); (225 9318500 A G); (226 9318582 G A); (227 9318584 G A); (228 9318682 T A); (229
9319112 C G); (230 9319421 T TA); (231 9319821 A G); (232 9319878 T A); (233 9320091 G T); (234
9320107 TTATA T); (235 9320144 T G); (236 9320368 A AT); (237 9320434 T C); (238 9320488 G A); (239
9320514 C T); (240 9320790 GA G); (241 9321074 T C); (242 9321284 T C); (243 9321369 G T); (244
9321439 G A); (245 9321706 A T); (246 9321900 G A); (247 9322060 GT G); (248 9322073 C A); (249
9322310 A C); (250 9322361 A C); (251 9322375 A ACCCACGATG); (252 9322434 T C); (253 9322662 A
G); (254 9322667 C T); (255 9323788 AGG A); (256 9323921 G A); (257 9324043 A G); (258 9324285 T
C); (259 9324748 A C); (260 9324814 T C); (261 9325202 C T); (262 9325253 A G); (263 9325415 A C); (264
9326743 G T); (265 9326764 A G); (266 9326857 T A); (267 9328391 T C); (268 9328946 T C); (269
9329079 C CT); (270 9329095 C T); (271 9329477 T G); (272 9329522 C T); (273 9329916 T C); (274
9330583 T C); (275 9330659 G A); (276 9330685 A G); (277 9331019 T C); (278 9331960 G A); (279
9332360 T C); (280 9332469 A C); (281 9333204 G A); (282 9333391 A AT); (283 9333539 C G); (284
9333657 G A); (285 9334350 T C); (286 9334549 A AT); (287 9334784 T C); (288 9334873 G A); (289
9334946 A G); (290 9335025 C T); (291 9335192 C G); (292 9335202 A G); (293 9335588 TA T); (294
9335684 T G); (295 9335750 C A); (296 9335859 A G); (297 9336658 A G); (298 9336724 G A); (299
9336930 T C); (300 9337090 T A); (301 9337643 A G); (302 9338549 C T); (303 9338644 C T); (304
9339150 C T); (305 9339438 A ACAAAATAAATAAATAATTGAATAAT); (306 9339593 C T); (307 9339699 C
T); (308 9339768 G A); (309 9339829 T C); (310 9340302 G A); (311 9340326 T C); (312 9340921 A
G); (313 9341328 G A); (314 9341768 G A); (315 9342188 C T); (316 9342315 G A); (317 9342331 G
T); (318 9343228 G T); (319 9343447 C T); (320 9343585 G A); (321 9343615 T C); (322 9343690 C
CA); (323 9343735 A G); (324 9344153 C T); (325 9344373 G A); (326 9344431 T C); (327 9344444 A
G); (328 9344866 C T); (329 9344979 G A); (330 9345231 A G); (331 9345406 C T); (332 9345511 A
G); (333 9345659 G A); (334 9345691 G A); (335 9345772 C G); (336 9346602 C T); (337 9346657 G
A); (338 9346848 A G); (339 9346895 A G); (340 9346932 G A); (341 9346956 A T); (342 9347477 C
A); (343 9347538 T C); (344 9347742 C A); (345 9348068 A G); (346 9348125 A G); (347 9348128 G
T); (348 9348652 A G); (349 9348949 C T); (350 9349015 A G); (351 9349036 G C); (352 9349106 G
A); (353 9349497 T C); (354 9349953 G A); (355 9350464 C T); (356 9350510 C T); (357 9350533 G A); (358
9350573 T C); (359 9350648 C T); (360 9351212 C T); (361 9351265 C T); (362 9351282 T A); (363
9351396 A G); (364 9351628 C T); (365 9351657 T C); (366 9352012 C T); (367 9352496 C T); (368
9352590 A G); (369 9352648 C T); (370 9352848 AG A); (371 9353102 T C); (372 9353182 G GC); (373
9353385 G A); (374 9353642 C T); (375 9353739 T C); (376 9355287 A G); (377 9355377 C T); (378
9355580 T C); (379 9355886 T C); (380 9356166 C T); (381 9356258 C T); (382 9356506 C T); (383
9356880 C G); (384 9357432 G T); (385 9357664 T C); (386 9357775 T C); (387 9358155 G A); (388
9358395 T A); (389 9358518 C T); (390 9358636 C T); (391 9358712 C A); (392 9358746 T C); (393
9359091 T G); (394 9359262 CA C); (395 9359423 A G); (396 9359564 C A); (397 9360107 T C); (398
9360188 A C); (399 9360295 C T); (400 9360391 C G); (401 9360596 C T); (402 9360747 C CT); (403
9360963 T A); (404 9361086 G A); (405 9361165 G A); (406 9361743 A C); (407 9362193 T C); (408
9362836 T C); (409 9363196 G A); (410 9363541 T G); (411 9364338 A G); (412 9364393 G T); (413
9364530 G A); (414 9364690 C T); (415 9364714 A T); (416 9364728 TGA T); (417 9364946 AT A); (418
9365048 C G); (419 9365069 T C); (420 9365105 A G); (421 9365170 C T); (422 9365282 C T); (423
9365422 T C); (424 9365761 A C); (425 9366289 G C); (426 9366787 G T); (427 9366839 T G); (428
9367003 G A); (429 9367058 A T); (430 9367611 AAAG A); (431 9367667 A T); (432 9367714 A T); (433
9369144 G T); (434 9369165 C A); (435 9369460 A C); (436 9369605 G A); (437 9370187 G C); (438
9370749 G A); (439 9370846 T A); (440 9370861 G A); (441 9371177 G A); (442 9371196 A G); (443
9371417 G T); (444 9371716 A G); (445 9371982 C A); (446 9372737 T G); (447 9373077 T C); (448
9373180 C T); (449 9373436 C T); (450 9374000 G A); (451 9374011 C T); (452 9374275 G A); (453
9374490 T C); (454 9374546 C T); (455 9374876 G A); (456 9374965 C T); (457 9375188 A G); (458
9375650 C T); (459 9376035 G A); (460 9376099 G A); (461 9376111 T C); (462 9376116 A G); (463
9376317 G A); (464 9376326 G A); (465 9376391 A G); (466 9377202 A G); (467 9378653 G A); (468
9378687 A G); (469 9378758 C T); (470 9378819 T C); (471 9378851 T C); (472 9378877 T C); (473
9378939 T C); (474 9379081 C T); (475 9379184 G A); (476 9379317 G A); (477 9379399 G A); (478
9379407 T C); (479 9379438 A G); (480 9379445 A G); (481 9379474 G T); (482 9379975 A G); (483
9380134 C T); (484 9380216 T C); (485 9380272 G A); (486 9380686 T C); (487 9381727 C T); (488
9381867 C T); (489 9381873 C T); (490 9381933 G A); (491 9382008 C T); (492 9382644 T C); (493
9382645 G A); (494 9382891 G T); (495 9382962 T C); (496 9383009 T C); (497 9383038 G A); (498
9383658 T G); (499 9383684 G A); (500 9383718 A T); (501 9383897 A T); (502 9383967 T G); (503
9384157 C T); (504 9384207 C T); (505 9384432 C T); (506 9385042 G A); (507 9385084 G A); (508
9385174 A G); (509 9385394 A G); (510 9385471 A G); (511 9385567 C T); (512 9385680 A G); (513
9386314 C A); (514 9386338 G T); (515 9386356 C T); (516 9386599 C T); (517 9388241 C T); (518
9388836 G GA); (519 9389115 G A); (520 9389584 G C); (521 9389646 C G); (522 9389753 T C); (523
9390011 C T); (524 9390035 T C); (525 9390233 T C); (526 9390454 G T); (527 9390522 T C); (528
9390578 G A); (529 9390643 T C); (530 9390826 C T); (531 9390965 A T); (532 9391244 T C); (533
9391287 C A); (534 9391414 A G); (535 9391671 G A); (536 9393285 G A); (537 9393297 A C); (538
9393426 C A); (539 9393551 T C); (540 9393559 G A); (541 9393628 G C); (542 9394563 A T); (543
9394624 A G); (544 9394954 A T); (545 9395187 C G); (546 9395688 G C); (547 9395755 T TA); (548
9396073 C T); (549 9396365 T C); (550 9396508 G T); (551 9396674 A T); (552 9396715 T C); (553
9397259 AAT A); (554 9397311 T A); (555 9397514 A G); (556 9397734 C A); (557 9398486 GA G); (558
9399145 G GA); (559 9399295 G A); (560 9399316 G A); (561 9399522 A G); (562 9400266 C A); (563
9400291 T A); (564 9400974 G A); (565 9401122 A T); (566 9401154 A G); (567 9401170 T
TTAATTAAAAAAACA); (568 9401282 T C); (569 9401457 C T); (570 9401508 C T); (571 9401600 A G); (572
9402229 C A); (573 9402238 G C); (574 9402255 C T); (575 9402833 T C); (576 9402872 G T); (577
9402998 G A); (578 9403120 T C); (579 9403622 A T); (580 9403658 G A); (581 9403684 T A); (582
9403729 G A); (583 9403741 C T); (584 9403825 G A); (585 9404146 A AGG); (586 9404163 G A); (587
9404375 T G); (588 9404395 G T); (589 9404448 G A); (590 9404713 G A); (591 9404743 C T); (592
9404759 G A); (593 9405051 T C); (594 9405081 C T); (595 9405407 G A); (596 9405413 C T); (597
9405419 A G); (598 9405421 A G); (599 9405764 G A); (600 9405780 GA G); (601 9405913 G C); (602
9406063 T C); (603 9406308 G A); (604 9406313 T C); (605 9406349 G A); (606 9406407 A G); (607
9406410 G A); (608 9406627 G A); (609 9406851 A G); (610 9406884 A G); (611 9406950 C T); (612
9406970 C T); (613 9407001 G A); (614 9407152 C T); (615 9407396 C T); (616 9407674 T G); (617
9407884 C T); (618 9407906 C T); (619 9407938 G A); (620 9408020 T A); (621 9408200 A G); (622
9408202 C T); (623 9408430 C T); (624 9408462 G C); (625 9409280 C T); (626 9409325 C CT); (627
9409347 A ATT); (628 9409378 G T); (629 9409400 A C); (630 9409403 C T); (631 9409411 T G); (632
9409708 T C); (633 9409766 T C); (634 9409991 G A); (635 9410020 T TA); (636 9410119 G A); (637
9410144 T C); (638 9410162 T A); (639 9410374 A G); (640 9410477 C T); (641 9410503 C G); (642
9410620 C T); (643 9410744 G A); (644 9410766 G A); (645 9410871 A G); (646 9410922 T C); (647
9411050 A G); (648 9411131 G A); (649 9411147 T C); (650 9411168 C A); (651 9411310 G A); (652
9411366 T C); (653 9411611 GA G); (654 9411830 TA T); (655 9412169 T A); (656 9412530 C T); (657
9412678 T A); (658 9413038 T C); (659 9413065 A G); (660 9413217 C T); (661 9413301 C A); (662
9413641 T C); (663 9413717 T C); (664 9413721 G A); (665 9414232 A G); (666 9414401 T C); (667
9414405 C T); (668 9414635 A G); (669 9414718 T C); (670 9414770 C T); (671 9414772 T C); (672
9414843 G A); (673 9415349 T C); (674 9415586 AAC A); (675 9415957 A G); (676 9416217 A G); (677
9416306 C G); (678 9416427 G A); (679 9416569 T C); (680 9416646 C T); (681 9416652 T C); (682
9416831 C A); (683 9416899 G C); (684 9416938 C T); (685 9417010 T C); (686 9417012 A G); (687
9417354 C T); (688 9417425 A G); (689 9417432 G A); (690 9418114 C T); (691 9418437 T C); (692
9418652 T C); (693 9419617 C T); (694 9419973 C T); (695 9420977 T A); (696 9421184 A G); (697
9421479 T C); (698 9421848 A G); (699 9421874 G A); (700 9421885 A C); (701 9421914 C T); (702
9422117 G A); (703 9422315 C T); (704 9422905 T C); (705 9422971 A G); (706 9423460 G A); (707
9423581 C G); (708 9423680 T G); (709 9424406 G A); (710 9424450 G A); (711 9424653 G A); (712
9424696 T G); (713 9424926 A T); (714 9425320 C T); (715 9425521 A G); (716 9425554 T C); (717
9425717 C T); (718 9425919 A G); (719 9425992 A G); (720 9426653 G A); (721 9426759 T C); (722
9427891 C T); (723 9427922 G A); (724 9427964 A G); (725 9428165 C A); (726 9428435 G A); (727
9428486 G T); (728 9428895 G A); (729 9428899 A G); (730 9429022 G A); (731 9429404 T G); (732
9429421 A G); (733 9429428 A G); (734 9429449 G A); (735 9429524 G A); (736 9429532 T C); (737
9429648 GTT G); (738 9429684 A G); (739 9429801 A AT); (740 9429883 T G); (741 9430012 G A); (742
9430118 C T); (743 9430119 A G); (744 9430468 C T); (745 9430514 C T); (746 9430589 C T); (747
9430776 C CT); (748 9430982 C G); (749 9431302 C T); (750 9431424 T A); (751 9431483 G A); (752
9431576 T C); (753 9432437 C G); (754 9432583 C G); (755 9432691 G A); (756 9432824 A AT); (757
9433535 C T); (758 9433664 G C); (759 9433809 T G); (760 9434341 A T); (761 9434558 T C); (762
9434653 C T); (763 9435162 C A); (764 9435398 A C); (765 9435450 T C); (766 9435461 T C); (767
9435753 A C); (768 9435767 T C); (769 9435821 C A); (770 9436059 T C); (771 9436160 A G); (772
9436307 T C); (773 9436802 C A); (774 9436930 C T); (775 9437053 C T); (776 9437153 G A); (777
9437246 G T); (778 9437284 T C); (779 9437421 C T); (780 9437441 G A); (781 9437442 C T); (782
9437510 G A); (783 9437525 A G); (784 9437557 C T); (785 9437697 A G); (786 9437852 C A); (787
9437870 C G); (788 9438099 TAGTTA T); (789 9438352 T C); (790 9438524 T C); (791 9438544 T C); (792
9439324 C G); (793 9439365 C T); (794 9439462 A G); (795 9439484 C G); (796 9440833 C G); (797
9440984 T C); (798 9440986 A G); (799 9441004 T C); (800 9441028 G C); (801 9441763 C T); (802
9442039 C T); (803 9442502 C T); (804 9442508 C T); (805 9442860 A T); (806 9442951 A G); (807
9443455 C A); (808 9443658 C T); (809 9444001 A G); (810 9444274 T C); (811 9445091 T G); (812
9445345 T C); (813 9445355 T C); (814 9445487 G A); (815 9445549 A G); (816 9446029 C A); (817
9446263 A C); (818 9446475 A T); (819 9446853 C T); (820 9446962 A G); (821 9447166 G C); (822
9447698 T C); (823 9447780 G A); (824 9448177 G A); (825 9448860 A T); (826 9449685 G T); (827
9450229 C T); (828 9451200 A G); (829 9451778 A C); (830 9452256 T TA); (831 9452390 A AT); (832
9452438 C T); (833 9452656 A AT); (834 9452719 T A); (835 9452807 T C); (836 9453097 A C); (837
9453275 G A); (838 9453294 T TA); (839 9453483 A G); (840 9453515 G A); (841 9453625 T C); (842
9453709 A G); (843 9453717 A G); (844 9454102 T C); (845 9454213 T C); (846 9454229 C T); (847
9454260 GA G); (848 9454295 G A); (849 9454312 T C); (850 9454326 GAATA G); (851 9454404 G A); (852
9454480 C T); (853 9454881 T G); (854 9454957 A G); (855 9454969 C T); (856 9455688 A G); (857
9455691 G A); (858 9455768 A C); (859 9455888 C T); (860 9456022 A T); (861 9456039 G A); (862
9456118 A T); (863 9456440 T C); (864 9456494 C A); (865 9456649 T G); (866 9456784 C T); (867
9457300 G A); (868 9457670 A C); (869 9457694 A C); (870 9457768 A T); (871 9457797 G A); (872
9457930 T G); (873 9457941 T C); (874 9458010 C T); (875 9458026 T C); (876 9458076 C T); (877
9458100 C CT); (878 9458177 G GGT); (879 9458216 T C); (880 9458217 A G); (881 9458322 C T); (882
9458440 G A); (883 9458792 G A); (884 9458826 T C); (885 9458827 C T); (886 9458864 G A); (887
9458868 C T); (888 9458872 T C); (889 9458895 C T); (890 9458897 T C); (891 9458908 C T); (892
9458926 T G); (893 9458953 T A); (894 9459175 T C); (895 9459191 C T); (896 9460328 C G); (897
9460416 A T); (898 9460654 A G); (899 9460785 A G); (900 9460840 G GCTGGGAATACTTTTTTT); (901
9461200 A T); (902 9461359 G A); (903 9461476 C T); (904 9461662 T A); (905 9462843 G GA); (906
9463111 T C); (907 9463724 G A); (908 9463725 T A); (909 9464187 CA C); (910 9464908 C T); (911
9465067 G GT); (912 9465073 A G); (913 9465130 G GA); (914 9465475 G C); (915 9465923 C T); (916
9466002 A G); (917 9466025 C T); (918 9466156 A C); (919 9466214 A G); (920 9466677 G T); (921
9467246 G A); (922 9467267 A T); (923 9467469 TA T); (924 9467791 A C); (925 9468047 T A); (926
9468288 G A); (927 9468290 G A); (928 9468292 G A); (929 9468294 G A); (930 9468296 G A); (931
9468298 G A); (932 9468326 A G); (933 9468332 A G); (934 9468472 C T); (935 9468538 G A); (936
9468690 A G); (937 9468751 C G); (938 9468766 A G); (939 9468776 C T); (940 9469027 T A); (941
9469599 C A); (942 9469752 GAT G); (943 9469788 C A); (944 9470182 G A); (945 9470605 T G); (946
9470702 C T); (947 9470724 A G); (948 9470740 T C); (949 9470754 A G); (950 9470763 C T); (951
9470794 G C); (952 9470804 C T); (953 9471060 T C); (954 9471073 G C); (955 9471199 C G); (956
9471261 A G); (957 9471377 C G); (958 9471460 T G); (959 9471556 G T); (960 9471911 C G); (961
9471990 C G); (962 9472128 A C); (963 9473195 C CA); (964 9473201 T G); (965 9473264 G C); (966
9473307 C T); (967 9473336 A G); (968 9473370 A G); (969 9473409 T C); (970 9473432 T A); (971
9473450 G A); (972 9473600 A G); (973 9473667 A G); (974 9474043 C T); (975 9474105 A T); (976
9474878 G C); (977 9474985 T C); (978 9475083 A G); (979 9475116 C T); (980 9475674 G T); (981
9475798 A AT); (982 9476065 G A); (983 9476151 G A); (984 9476444 G A); (985 9476886 G A); (986
9476893 T C); (987 9476963 C T); (988 9477262 G A); (989 9477462 G A); (990 9477821 C T); (991
9477936 C T); (992 9478024 T G); (993 9478191 C T); (994 9478379 G A); (995 9478393 A G); (996
9478465 G T); (997 9478480 G A); (998 9478550 G A); (999 9478599 A G); (1000 9478776 T C); (1001
9478938 GA G); (1002 9479507 C T); (1003 9479765 G A); (1004 9479834 C T); (1005 9480013 C A); (1006
9480027 C T); (1007 9480171 C T); (1008 9480229 G A); (1009 9480394 G A); (1010 9480558 C
CAT); (1011 9481238 A C); (1012 9481265 G A); (1013 9481669 C T); (1014 9481717 C T); (1015 9481748
A G); (1016 9481789 C G); (1017 9481809 C T); (1018 9482008 G A); (1019 9482170 T C); (1020 9482174
C T); (1021 9482276 T C); (1022 9482465 G A); (1023 9483063 T C); (1024 9483106 C T); (1025 9483109 C
G); (1026 9483127 T C); (1027 9483206 T C); (1028 9483237 A G); (1029 9483288 A AG); (1030 9483298
G A); (1031 9483431 T C); (1032 9483497 C T); (1033 9483574 T C); (1034 9483831 C T); (1035 9483837 C
T); (1036 9483915 C T); (1037 9484496 G A); (1038 9484498 C T); (1039 9484568 C A); (1040 9484610 G
T); (1041 9484738 G A); (1042 9484752 G A); (1043 9484936 A G); (1044 9484954 C T); (1045 9484991 T
C); (1046 9485422 CTT C); (1047 9485469 G A); (1048 9485523 C A); (1049 9485539 A G); (1050 9485579
T C); (1051 9485689 C T); (1052 9485853 T A); (1053 9486124 G A); (1054 9486180 A G); (1055 9486391
A G); (1056 9486394 A G); (1057 9486513 C T); (1058 9486515 C T); (1059 9486535 C
CATATATATATATATAT); (1060 9486586 A T); (1061 9486593 T A); (1062 9486648 C CAT); (1063 9486700
T C); (1064 9486735 T TGGATAACTAGAA); (1065 9486785 T C); (1066 9487413 C T); (1067 9487474 A
T); (1068 9487566 T A); (1069 9487683 A G); (1070 9487759 G A); (1071 9487777 A AAACCAGTT); (1072
9487884 G A); (1073 9488114 T A); (1074 9488203 C T); (1075 9488210 C G); (1076 9488232 C T); (1077
9488248 G A); (1078 9488283 G A); (1079 9488452 G A); (1080 9488495 T C); (1081 9488587 T C); (1082
9488616 G A); (1083 9488678 C T); (1084 9488873 C G); (1085 9488893 A G); (1086 9489041 A G); (1087
9489077 A T); (1088 9489144 C G); (1089 9489275 A G); (1090 9489301 G A); (1091 9489515 A G); (1092
9489535 A G); (1093 9490274 A G); (1094 9490687 T C); (1095 9491264 T C); (1096 9491304 C T); (1097
9491446 C T); (1098 9491495 T C); (1099 9491887 G A); (1100 9492012 A G); (1101 9492034 C T); (1102
9492089 A C); (1103 9492103 G A); (1104 9492316 T C); (1105 9492403 G A); (1106 9492610 G A); (1107
9492727 A G); (1108 9492949 GT G); (1109 9492962 G A); (1110 9493014 A T); (1111 9493447 C
T); (1112 9493477 G C); (1113 9493610 AAAG A); (1114 9493660 T C); (1115 9493754 T TATA); (1116
9493899 G C); (1117 9493953 C T); (1118 9493970 T C); (1119 9495556 T G); (1120 9495789 G A); (1121
9496822 T C); (1122 9496834 C A); (1123 9497339 A T); (1124 9497345 C T); (1125 9497459 C T); (1126
9497670 T C); (1127 9497771 C T); (1128 9498193 A G); (1129 9498199 G A); (1130 9498291 C T); (1131
9498377 A C); (1132 9498551 A T); (1133 9498613 C A); (1134 9498665 A C); (1135 9498693 A
AAAATTC); (1136 9498699 T G); (1137 9498700 T A); (1138 9498762 G A); (1139 9498766 G A); (1140
9498769 C T); (1141 9498779 T C); (1142 9498809 G A); (1143 9498811 A G); (1144 9498878 T C); (1145
9498927 A G); (1146 9499155 C T); (1147 9499438 G C); (1148 9500102 C A); (1149 9500312 T C); (1150
9501828 C T); (1151 9502018 T C); (1152 9502027 A G); (1153 9502088 T C); (1154 9502837 A C); (1155
9502845 A G); (1156 9503198 A T); (1157 9503199 A G); (1158 9503453 T C); (1159 9503766 A G); (1160
9503776 A G); (1161 9503842 T C); (1162 9503850 T C); (1163 9503917 A G); (1164 9504014 G A); (1165
9504037 G A); (1166 9504099 A G); (1167 9504104 G T); (1168 9504182 T C); (1169 9504194 C T); (1170
9504221 T C); (1171 9504299 C T); (1172 9504304 A G); (1173 9504375 A T); (1174 9504424 C T); (1175
9504436 A G); (1176 9504444 C T); (1177 9504498 A G); (1178 9504499 C T); (1179 9504586 A G); (1180
9504600 C T); (1181 9504695 T C); (1182 9504807 C A); (1183 9505048 A G); (1184 9505115 G
GAA); (1185 9505179 T C); (1186 9505366 C T); (1187 9505560 C T); (1188 9505789 C T); (1189 9505820
A T); (1190 9506143 C T); (1191 9506539 C T); (1192 9506789 A G); (1193 9506983 G A); (1194 9507135
C T); (1195 9507228 C A); (1196 9507985 A G); (1197 9507996 A C); (1198 9508018 C G); (1199 9508045
C T); (1200 9508063 G T); (1201 9508214 T C); (1202 9508355 A G); (1203 9508413 G A); (1204 9508647
G A); (1205 9508675 C A); (1206 9508676 C A); (1207 9508703 T C); (1208 9508748 T C); (1209 9508761
A G); (1210 9508814 G A); (1211 9508950 A AT); (1212 9508956 G A); (1213 9508990 G A); (1214
9509040 C G); (1215 9509041 T C); (1216 9509251 G A); (1217 9509257 G C); (1218 9509309 A
AC); (1219 9509974 A G); (1220 9510072 T C); (1221 9510170 A C); (1222 9510228 T C); (1223 9510259 T
C); (1224 9511307 A G); (1225 9511388 C T); (1226 9511499 C T); (1227 9511699 G A); (1228 9511710 A
G); (1229 9511735 T C); (1230 9511819 G GCGGTA); (1231 9511931 G T); (1232 9511935 C T); (1233
9512066 A G); (1234 9512088 T C); (1235 9512155 A G); (1236 9512190 T C); (1237 9512201 C T); (1238
9512346 G A); (1239 9512394 C T); (1240 9513054 A G); (1241 9513098 A C); (1242 9513099 T A); (1243
9513102 G T); (1244 9513103 T G); (1245 9513288 TAAG T); (1246 9514033 C CT); (1247 9514398 A
G); (1248 9514458 CTT C); (1249 9514698 A AT); (1250 9514729 T G); (1251 9514960 G A); (1252
9514961 A G); (1253 9514964 T A); (1254 9514965 A T); (1255 9514967 T A); (1256 9515964 G C); (1257
9516118 A C); (1258 9517218 G C); (1259 9518352 T C); (1260 9518782 A C); (1261 9518814 G T); (1262
9518901 G A); (1263 9518911 G A); (1264 9519142 C T); (1265 9519974 T G); (1266 9520537 C T); (1267
9520548 C T); (1268 9520622 G A); (1269 9520716 T C); (1270 9520831 C T); (1271 9520998 G A); (1272
9523230 A C); (1273 9525197 TTATATA T); (1274 9525277 T A); (1275 9525623 C T); (1276 9526027 C
G); (1277 9526860 T C); (1278 9526915 A G); (1279 9526978 T C); (1280 9526996 A G); (1281 9526998 G
T); (1282 9527037 C T); (1283 9527171 G A); (1284 9527386 A C); (1285 9527707 T C); (1286 9527945 T
C); (1287 9528150 T C); (1288 9528154 G A); (1289 9528433 G C); (1290 9528505 G A); (1291 9528549 C
T); (1292 9528569 C T); (1293 9528617 G A); (1294 9528977 T C); (1295 9529022 C T); (1296 9529190 G
A); (1297 9529288 C T); (1298 9531362 G A); (1299 9531521 C G); (1300 9531589 A G); (1301 9532233
AT A); (1302 9532520 C CATAT); (1303 9533329 A T); (1304 9533441 C A); (1305 9534097 A T); (1306
9534241 C G); (1307 9534463 TG T); (1308 9535274 G GA); (1309 9535309 G A); (1310 9535436 C
G); (1311 9535582 C A); (1312 9535652 C T); (1313 9535660 G A); (1314 9535719 C T); (1315 9535798 T
C); (1316 9536221 C T); (1317 9536360 C T); (1318 9536367 A G); (1319 9536511 C T); (1320 9536648 A
T); (1321 9536863 G A); (1322 9537084 A G); (1323 9537281 A T); (1324 9537334 T C); (1325 9537345 G
A); (1326 9537442 T G); (1327 9538176 A T); (1328 9538292 C T); (1329 9538639 G A); (1330 9538645 C
T); (1331 9538715 C T); (1332 9538718 G A); (1333 9538744 G A); (1334 9538774 C A); (1335 9538923 A
G); (1336 9538950 T C); (1337 9538968 G A); (1338 9538980 C CTGTTAT); (1339 9538991 C T); (1340
9538992 C T); (1341 9539051 C T); (1342 9539063 G A); (1343 9539074 C T); (1344 9539097 T A); (1345
9539114 C G); (1346 9539115 T C); (1347 9544888 TCTTCTCCGGCGC T); (1348 9544891 T C); (1349
9544895 T C); (1350 9544929 C T); (1351 9544970 C G); (1352 9545002 G A); (1353 9545005 C T); (1354
9546595 AT A); (1355 9548626 TA T); (1356 9548753 T A); (1357 9548754 T A); (1358 9550143 T
C); (1359 9550298 G GT); (1360 9551455 G C); (1361 9551798 C T); (1362 9554823 T A); (1363 9556253 G
A); (1364 9556385 A T); (1365 9556386 C A); (1366 9556387 A T); (1367 9558434 G A); (1368 9559782 C
T); (1369 9560469 C T); (1370 9562241 G T); (1371 9562375 G A); (1372 9562572 AG A); (1373 9563011 C
T); (1374 9563060 C A); (1375 9563143 G A); (1376 9563696 C T); (1377 9565117 G A); (1378 9565539 C
CA); (1379 9565607 G A); (1380 9565743 C T); (1381 9570557 A T); (1382 9570604 A G); (1383 9571083 C
T); (1384 9571204 C T); (1385 9571338 G A); (1386 9571373 T C); (1387 9571381 C A); (1388 9571391 A
G); (1389 9572908 TTTCTTCCTAC T); (1390 9576136 G A); (1391 9576140 C A); (1392 9576141 T C); (1393
9576183 A T); (1394 9576192 C T); (1395 9576236 C CA); (1396 9576279 C T); (1397 9576355 A T); (1398
9576420 G A); (1399 9576479 C T); (1400 9576758 G A); (1401 9576894 C T); (1402 9577147 G A); (1403
9577219 A T); (1404 9578749 C T); (1405 9578841 G A); (1406 9578892 C T); (1407 9578908 T G); (1408
9578966 G A); (1409 9583392 T A); (1410 9584080 T A); (1411 9584596 G A); (1412 9584994 G T); (1413
9585677 T TTA); (1414 9585956 C T); (1415 9586568 G A); (1416 9587369 G A); (1417 9587448 G
A); (1418 9587872 G A); (1419 9587881 C T); (1420 9588310 T G); (1421 9588543 T C); (1422 9588861 A
C); (1423 9589152 G GA); (1424 9590402 A C); (1425 9594279 C G); (1426 9596514 G A); (1427 9596676
G A); (1428 9597148 T G); (1429 9605988 G C); (1430 9607748 C T); (1431 9607813 A C); (1432 9608272
T A); (1433 9608382 A C); (1434 9608414 T C); (1435 9608525 C T); (1436 9608997 C G); (1437 9609015 C
T); (1438 9609276 C A); (1439 9609318 C G); (1440 9609572 C T); (1441 9609606 CATGAT C); (1442
9610804 G A); (1443 9611360 C A); (1444 9611414 G T); (1445 9611483 G A); (1446 9612311 C T); (1447
9612505 G A); (1448 9612635 C T); (1449 9612678 G A); (1450 9612707 G A); (1451 9614094 A
AT); (1452 9614815 A C); (1453 9615257 T C); (1454 9615292 A T); (1455 9615734 A C); (1456 9616858 A
AT); (1457 9617712 C A); (1458 9618036 T A); (1459 9618346 T C); (1460 9618390 AT A); (1461 9618625
A C); (1462 9618803 A T); (1463 9619001 A C); (1464 9620803 A C); (1465 9621209 G A); (1466 9621982
A G); (1467 9622088 A G); (1468 9622760 T G); (1469 9622986 G T); (1470 9623977 G A); (1471 9623997
C T); (1472 9624189 C T); (1473 9624824 G GA); (1474 9625058 C T); (1475 9625308 G A); (1476 9626222
T C); (1477 9626537 C A); (1478 9626627 G A); (1479 9626656 A T); (1480 9626876 A G); (1481 9626928
G T); (1482 9627665 C T); (1483 9627697 A G); (1484 9627976 G A); (1485 9628295 C G); (1486 9628491
C T); (1487 9628493 C T); (1488 9628498 G A); (1489 9628640 A C); (1490 9628810 T C); (1491 9628819 C
T); (1492 9628865 A G); (1493 9628909 C CT); (1494 9628934 A G); (1495 9628959 A C); (1496 9629112 T
A); (1497 9629269 G A); (1498 9629575 G A); (1499 9629700 C T); (1500 9629704 C A); (1501 9629739 C
T); (1502 9629748 C T); (1503 9629875 A G); (1504 9630045 C A); (1505 9630353 A G); (1506 9630783 C
T); (1507 9630882 T G); (1508 9631000 C T); (1509 9631068 G A); (1510 9631091 C T); (1511 9631397 A
G); (1512 9631496 G A); (1513 9631808 C T); (1514 9631836 G A); (1515 9631952 A G); (1516 9632140 T
C); (1517 9632186 G A); (1518 9632720 A G); (1519 9632866 C T); (1520 9632882 C T); (1521 9632897 T
C); (1522 9632962 A G); (1523 9632976 T C); (1524 9633027 G T); (1525 9634720 T C); (1526 9634806 G
C); (1527 9634825 A G); (1528 9634983 C T); (1529 9635321 G A); (1530 9635376 A G); (1531 9635427 A
G); (1532 9635577 T C); (1533 9635632 T C); (1534 9635658 G C); (1535 9635819 A G); (1536 9635966 C
T); (1537 9636210 T C); (1538 9636314 A G); (1539 9636357 G A); (1540 9636428 A G); (1541 9636457 T
C); (1542 9636976 A G); (1543 9637145 T C); (1544 9637171 A C); (1545 9637522 T C); (1546 9637591 A
G); (1547 9637628 G A); (1548 9637674 C T); (1549 9638002 C T); (1550 9638018 C T); (1551 9638086 G
A); (1552 9638092 A C); (1553 9638169 C T); (1554 9638171 G C); (1555 9638242 T C); (1556 9638274 A
AG); (1557 9638462 C T); (1558 9638526 C T); (1559 9638637 C T); (1560 9638710 G A); (1561 9638774 T
C); (1562 9639378 C T); (1563 9639525 T G); (1564 9640834 G A); (1565 9641144 A G); (1566 9641357 T
TTTA); (1567 9642630 G T); (1568 9643058 G A); (1569 9643942 A G); (1570 9643945 G A); (1571
9643993 A G); (1572 9644031 C T); (1573 9644046 C T); (1574 9644067 C T); (1575 9644651 T C); (1576
9644725 A T); (1577 9645020 A G); (1578 9645278 T TA); (1579 9645337 T C); (1580 9645440 G A); (1581
9645471 C T); (1582 9646011 C A); (1583 9646102 G C); (1584 9646346 A G); (1585 9646612 G C); (1586
9647217 T C); (1587 9647412 G A); (1588 9647617 T A); (1589 9647943 A G); (1590 9648336 T A); (1591
9648715 T TA); (1592 9648754 G A); (1593 9649050 T A); (1594 9649234 T C); (1595 9649501 G A); (1596
9649570 T C); (1597 9649641 C T); (1598 9649750 T G); (1599 9649928 A C); (1600 9650449 A T); (1601
9650492 A G); (1602 9650977 A AC); (1603 9651899 T C); (1604 9652280 A G); (1605 9652575 T C); (1606
9652805 A G); (1607 9652865 A C); (1608 9652915 G A); (1609 9652951 A G); (1610 9652957 C T); (1611
9653130 T C); (1612 9653152 G A); (1613 9653440 C T); (1614 9654318 C T); (1615 9654524 A G); (1616
9654552 G A); (1617 9654576 C A); (1618 9654687 G A); (1619 9655793 C T); (1620 9656007 G A); (1621
9656338 A G); (1622 9656363 A C); (1623 9656508 T TG); (1624 9656511 C T); (1625 9656730 G A); (1626
9656796 C T); (1627 9656808 A G); (1628 9656826 T C); (1629 9657055 T C); (1630 9657227 A C); (1631
9657451 G A); (1632 9657916 A G); (1633 9658690 T C); (1634 9658695 G A); (1635 9658723 C T); (1636
9658776 A G); (1637 9658952 A G); (1638 9660024 G A); (1639 9660225 G A); (1640 9661058 G A); (1641
9661093 C CACTTCCCTCCTTTAAA); (1642 9661176 C A); (1643 9661419 G A); (1644 9661567 T G); (1645
9661592 G A); (1646 9661881 AAAG A); (1647 9662044 GA G); (1648 9662204 CAT C); (1649 9662363 T
A); (1650 9662528 C A); (1651 9662716 AT A); (1652 9663388 G A); (1653 9663392 T C); (1654 9663603 G
A); (1655 9663689 A G); (1656 9663725 C T); (1657 9663791 C T); (1658 9663805 A T); (1659 9663827 G
A); (1660 9663997 A C); (1661 9664928 A G); (1662 9664942 A G); (1663 9664993 T C); (1664 9665149 C
T); (1665 9665175 T C); (1666 9665451 G T); (1667 9665464 G A); (1668 9665521 C T); (1669 9665564 A
G); (1670 9665721 G A); (1671 9665737 A G); (1672 9665773 T G); (1673 9665779 G A); (1674 9665790 C
T); (1675 9665870 C T); (1676 9665879 A G); (1677 9665971 A T); (1678 9666010 C A); (1679 9666017 G
A); (1680 9666175 A G); (1681 9666189 T C); (1682 9666251 G GGGTGGCGCCAGTTGTGGCGCT); (1683
9666253 A G); (1684 9666261 A C); (1685 9666462 G A); (1686 9666570 C A); (1687 9666676 A G); (1688
9666685 G T); (1689 9666687 A G); (1690 9666718 A G); (1691 9666894 A G); (1692 9667039 G A); (1693
9667041 G T); (1694 9668015 C G); (1695 9668290 G A); (1696 9668292 G A); (1697 9668478 T C); (1698
9668613 G GA); (1699 9668782 T C); (1700 9668833 G A); (1701 9668958 C A); (1702 9668961 A
G); (1703 9669128 G A); (1704 9669150 T C); (1705 9669413 C T); (1706 9669454 T C); (1707 9669494 C
T); (1708 9669512 C G); (1709 9669532 T C); (1710 9669740 T A); (1711 9669814 T C); (1712 9669815 G
T); (1713 9670067 G A); (1714 9670082 G A); (1715 9670088 C T); (1716 9670135 A G); (1717 9670153 G
A); (1718 9670162 G A); (1719 9670232 A G); (1720 9670240 C T); (1721 9670257 G A); (1722 9670259 T
C); (1723 9670393 C G); (1724 9670413 A G); (1725 9670846 G A); (1726 9671110 T C); (1727 9671206 T
C); (1728 9671360 G A); (1729 9671477 G A); (1730 9671511 G A); (1731 9671835 A G); (1732 9671841
GA G); (1733 9671901 G A); (1734 9671924 A C); (1735 9672161 G A); (1736 9672845 T C); (1737
9672856 G A); (1738 9673574 G A); (1739 9673771 A G); (1740 9673983 C G); (1741 9674663 T C); (1742
9674676 T C); (1743 9674688 T C); (1744 9674710 A C); (1745 9675203 A ATGTG); (1746 9675415 G
T); (1747 9675474 A C); (1748 9675475 T A); (1749 9675481 ATT A); (1750 9675490 G A); (1751 9675744
A G); (1752 9675967 T C); (1753 9676084 G C); (1754 9676091 T C); (1755 9676152 A T); (1756 9676598 T
A); (1757 9676857 T G); (1758 9677609 C A); (1759 9677862 C T); (1760 9678021 C T); (1761 9678283 T
C); (1762 9678536 T A); (1763 9678799 A T); (1764 9679098 G A); (1765 9679182 G A); (1766 9679273 G
T); (1767 9679291 A C); (1768 9679389 G A); (1769 9679955 G A); (1770 9680000 CT C); (1771 9680224 A
C); (1772 9680244 G A); (1773 9680837 A G); (1774 9680868 G T); (1775 9680869 G T); (1776 9680894 C
T); (1777 9681251 C T); (1778 9681361 C T); (1779 9681715 A C); (1780 9681769 T G); (1781 9681963 A
C); (1782 9682235 G A); (1783 9682407 A G); (1784 9682433 T C); (1785 9682467 A G); (1786 9682552 C
T); (1787 9682841 T A); (1788 9682870 T C); (1789 9682904 G A); (1790 9682932 T C); (1791 9683157 A
G); (1792 9683185 T C); (1793 9683531 G A); (1794 9683588 A G); (1795 9683759 C A); (1796 9683867 T
C); (1797 9683899 A G); (1798 9684151 G A); (1799 9684248 C A); (1800 9684251 G T); (1801 9684440 C
G); (1802 9684475 C A); (1803 9684663 A C); (1804 9684934 G A); (1805 9685182 A C); (1806 9685417 T
C); (1807 9685531 C T); (1808 9685577 G A); (1809 9685890 A C); (1810 9685933 T C); (1811 9686034 C
G); (1812 9686045 G A); (1813 9686219 G A); (1814 9686220 A G); (1815 9686342 G A); (1816 9686343 A
G); (1817 9686712 C T); (1818 9686860 T G); (1819 9686964 T C); (1820 9687026 T C); (1821 9687048 G
T); (1822 9687457 G A); (1823 9687937 C G); (1824 9688071 C A); (1825 9688241 T C); (1826 9688244 T
C); (1827 9688267 G C); (1828 9688420 A G); (1829 9689086 G A); (1830 9689208 T C); (1831 9689304 A
T); (1832 9689472 G A); (1833 9689504 G C); (1834 9689663 C T); (1835 9689804 G A); (1836 9689931 G
A); (1837 9690056 C T); (1838 9690264 T TA); (1839 9690265 G A); (1840 9690562 A C); (1841 9690591 T
C); (1842 9690794 C T); (1843 9690934 T C); (1844 9690979 G A); (1845 9690987 G A); (1846 9690989 T
C); (1847 9691103 G A); (1848 9691394 A G); (1849 9691837 A G); (1850 9691882 T C); (1851 9691914 A
G); (1852 9692003 G A); (1853 9692143 A G); (1854 9692416 C CG); (1855 9692442 T G); (1856 9692454
A G); (1857 9692739 G A); (1858 9692790 G A); (1859 9692810 G A); (1860 9693019 A G); (1861 9693056
T C); (1862 9693337 G A); (1863 9693387 A G); (1864 9693518 C T); (1865 9693545 G A); (1866 9693587
T G); (1867 9693598 T C); (1868 9694127 G C); (1869 9694166 C T); (1870 9694258 G A); (1871 9694288
G GACA); (1872 9694515 G GTTATAATTATTTA); (1873 9694526 G T); (1874 9694749 G A); (1875 9694884
C T); (1876 9694969 C T); (1877 9695114 G T); (1878 9695117 G A); (1879 9695162 G A); (1880 9695420
A T); (1881 9695444 G A); (1882 9695481 C A); (1883 9695820 A G); (1884 9696130 G A); (1885 9696193
C A); (1886 9696239 T C); (1887 9696287 A T); (1888 9696735 A G); (1889 9697249 A C); (1890 9697500
C T); (1891 9697766 T A); (1892 9697980 G C); (1893 9698012 G A); (1894 9698030 C G); (1895 9698177
CA C); (1896 9699128 G T); (1897 9699181 G A); (1898 9699463 C T); (1899 9699503 G A); (1900 9699538
G A); (1901 9699891 G C); (1902 9700209 G A); (1903 9700450 C T); (1904 9700464 T A); (1905 9700637
T TTTCTTCTTCTTCTTCTTCTTCTTC); (1906 9700979 T G); (1907 9701043 T A); (1908 9701453 G C); (1909
9701797 T C); (1910 9702063 A G); (1911 9702105 A G); (1912 9702865 A G); (1913 9703225 A G); (1914
9703424 C G); (1915 9703480 A AAATCAG); (1916 9703502 T A); (1917 9703556 G A); (1918 9703591 G
C); (1919 9703622 A G); (1920 9703629 G T); (1921 9703634 G T); (1922 9704124 A T); (1923 9704202 T
C); (1924 9704281 T C); (1925 9704342 T C); (1926 9704510 A G); (1927 9704619 G A); (1928 9704782 A
G); (1929 9705733 A G); (1930 9706026 G GATTGCATAATAGAACTTTAAAAAACATAAA); (1931 9706142 G
T); (1932 9706178 T C); (1933 9706205 C T); (1934 9706210 C G); (1935 9706540 T A); (1936 9707093 A
G); (1937 9707196 A G); (1938 9707214 C T); (1939 9707307 A G); (1940 9707537 A C); (1941 9707618 A
T); (1942 9707688 T C); (1943 9707714 TA T); (1944 9707789 A G); (1945 9707865 C T); (1946 9708027 G
A); (1947 9708044 AG A); (1948 9708074 C T); (1949 9708304 A C); (1950 9708328 T C); (1951 9708365 A
G); (1952 9709156 C T); (1953 9709607 C A); (1954 9710169 T C); (1955 9710236 A C); (1956 9710249 C
A); (1957 9710294 T C); (1958 9710766 C T); (1959 9710868 AG A); (1960 9710906 C A); (1961 9710914 G
A); (1962 9710923 T C); (1963 9711037 A G); (1964 9711157 C A); (1965 9711428 A G); (1966 9711455 A
G); (1967 9711957 A T); (1968 9711964 C T); (1969 9711982 A G); (1970 9712039 A G); (1971 9712070 C
T); (1972 9712667 C T); (1973 9713457 G A); (1974 9713772 A G); (1975 9713832 A G); (1976 9714117 T
TTCACAACATGAAAATC); (1977 9714385 A G); (1978 9714759 T C); (1979 9714817 AG A); (1980 9714886
G A); (1981 9714941 T C); (1982 9715280 G GTT); (1983 9715318 G T); (1984 9715495 C T); (1985
9715738 CTG C); (1986 9715887 T G); (1987 9716140 T C); (1988 9716185 G T); (1989 9716302 G
A); (1990 9716629 A T); (1991 9716648 A C); (1992 9716804 A T); (1993 9717220 G A); (1994 9717531 A
G); (1995 9717985 A G); (1996 9718115 C T); (1997 9718207 C T); (1998 9718341 C G); (1999 9718429 G
C); (2000 9718670 G GA); (2001 9718749 G T); (2002 9718793 A AC); (2003 9719396 A G); (2004 9719704
G A); (2005 9719823 A G); (2006 9719971 A G); (2007 9720350 C G); (2008 9720394 C A); (2009 9720399
G A); (2010 9720897 T C); (2011 9721013 C T); (2012 9721044 G A); (2013 9721102 A G); (2014 9721291
C T); (2015 9721330 C T); (2016 9721412 T C); (2017 9721548 C T); (2018 9721558 A
AATGCAGAATCAATT); (2019 9721878 G A); (2020 9721941 A T); (2021 9722022 T A); (2022 9722116 G
A); (2023 9722172 T C); (2024 9722212 A G); (2025 9722421 T C); (2026 9722612 C T); (2027 9722637 C
T); (2028 9722829 T C); (2029 9722922 T C); (2030 9723286 T C); (2031 9723297 T C); (2032 9723595 A
G); (2033 9723599 G A); (2034 9723620 T G); (2035 9723986 C T); (2036 9724085 C T); (2037 9724086 G
C); (2038 9724142 AT A); (2039 9724371 T A); (2040 9724373 A T); (2041 9725330 G C); (2042 9725799 G
A); (2043 9725827 G A); (2044 9725893 T C); (2045 9726581 G A); (2046 9726723 G C); (2047 9726749
AATCAGAAAGTCGG A); (2048 9726765 C T); (2049 9726796 G A); (2050 9726799 T C); (2051 9727046 C
T); (2052 9727103 A G); (2053 9727111 G A); (2054 9727119 G A); (2055 9727136 GA G); (2056 9727225
C T); (2057 9727232 A T); (2058 9727237 C CT); (2059 9727284 A G); (2060 9727324 G A); (2061 9727356
T C); (2062 9727401 G A); (2063 9727500 C T); (2064 9727644 T G); (2065 9727864 A C); (2066 9727919
C T); (2067 9727991 C T); (2068 9728047 T G); (2069 9728253 A G); (2070 9728303 A G); (2071 9728333
G C); (2072 9728406 T C); (2073 9728420 T TTATATATATA); (2074 9728445 A G); (2075 9728600 G
A); (2076 9729120 T G); (2077 9729191 G C); (2078 9729397 A AT); (2079 9729465 A T); (2080 9729846 A
C); (2081 9729911 G A); (2082 9730519 C T); (2083 9730558 T TA); (2084 9730584 G A); (2085 9730600 C
T); (2086 9730622 G A); (2087 9730831 T C); (2088 9730916 C T); (2089 9731075 G A); (2090 9731123 A
T); (2091 9731277 A C); (2092 9731291 A G); (2093 9731420 G A); (2094 9731471 TA T); (2095 9731888
AT A); (2096 9732083 G GTT); (2097 9732299 C A); (2098 9732353 A G); (2099 9732354 C T); (2100
9732370 A G); (2101 9732441 C T); (2102 9732469 C A); (2103 9732662 A G); (2104 9732693 T C); (2105
9732758 A G); (2106 9732837 G A); (2107 9733040 C T); (2108 9733068 CA C); (2109 9733118 G
A); (2110 9733786 T C); (2111 9733942 T C); (2112 9734246 AT A); (2113 9734317 G A); (2114 9734740 C
A); (2115 9734878 A T); (2116 9735161 A T); (2117 9735371 A T); (2118 9735481 AT A); (2119 9735640 A
C); (2120 9735666 A T); (2121 9735679 T C); (2122 9735747 A T); (2123 9735998 C T); (2124 9736327 T
C); (2125 9736448 C A); (2126 9736512 C T); (2127 9736554 T C); (2128 9736605 C CA); (2129 9737293 T
C); (2130 9737319 A C); (2131 9737469 T C); (2132 9737497 A G); (2133 9737507 T C); (2134 9738139 T
A); (2135 9738631 G A); (2136 9738646 A C); (2137 9738748 G A); (2138 9738943 C A); (2139 9738975 C
T); (2140 9739706 A G); (2141 9739709 C T); (2142 9739734 T A); (2143 9739805 A G); (2144 9739807 T
G); (2145 9739942 T C); (2146 9739968 A G); (2147 9740019 G A); (2148 9740340 G A); (2149 9740857 G
C); (2150 9740919 C T); (2151 9740937 C T); (2152 9741194 A G); (2153 9741726 C T); (2154 9743143 A
G); (2155 9743316 G A); (2156 9744844 A G); (2157 9744955 C CTCACCTTGTTG); (2158 9744960 T
C); (2159 9744987 A C); (2160 9745001 A G); (2161 9745022 A T); (2162 9745495 A G); (2163 9745767 A
G); (2164 9745852 C T); (2165 9745869 T C); (2166 9745926 C A); (2167 9745985 C T); (2168 9746281 T
C); (2169 9746790 G A); (2170 9746825 G A); (2171 9746955 G A); (2172 9747149 C T); (2173 9747218 C
CT); (2174 9748048 A C); (2175 9748308 T C); (2176 9748365 A AG); (2177 9748591 T C); (2178 9748650
C T); (2179 9748769 T C); (2180 9748859 T C); (2181 9748905 C A); (2182 9749089 T C); (2183 9749178 G
T); (2184 9749195 G A); (2185 9749229 C G); (2186 9749539 T G); (2187 9766662 T C); (2188 9766788 G
A); (2189 9766859 C A); (2190 9767100 G A); (2191 9767124 T C); (2192 9767285 A G); (2193 9767519 A
G); (2194 9767753 C T); (2195 9767799 G A); (2196 9768102 T C); (2197 9768203 T G); (2198 9768426 A
C); (2199 9768471 T C); (2200 9768828 G A); (2201 9768931 G A); (2202 9769267 G T); (2203 9769435 G
A); (2204 9769878 A AGTGGCTTGGGCATGGTTT); (2205 9770022 G A); (2206 9770090 GA G); (2207
9770247 T C); (2208 9770430 G A); (2209 9770538 A C); (2210 9770562 A C); (2211 9770962 C G); (2212
9771115 T C); (2213 9771131 T G); (2214 9771236 T C); (2215 9771239 T C); (2216 9771318 G C); (2217
9771887 T C); (2218 9771947 T A); (2219 9772063 G A); (2220 9772096 C A); (2221 9772171 G A); (2222
9772237 T C); (2223 9772281 G A); (2224 9772282 A G); (2225 9772364 T C); (2226 9772547 G A); (2227
9772560 T C); (2228 9772766 T C); (2229 9772786 A G); (2230 9772792 C T); (2231 9772793 A G); (2232
9772794 C T); (2233 9772824 C T); (2234 9772842 T C); (2235 9772884 A G); (2236 9772910 C T); (2237
9772985 A G); (2238 9773977 A G); (2239 9774096 A T); (2240 9774458 C T); (2241 9774650 C T); (2242
9774744 G A); (2243 9774755 G T); (2244 9774757 C T); (2245 9774771 C T); (2246 9774912 A C); (2247
9774982 A C); (2248 9775019 G A); (2249 9775339 G C); (2250 9775343 T C); (2251 9775528 T C); (2252
9775586 G A); (2253 9775654 A T); (2254 9775747 T C); (2255 9775854 G A); (2256 9776066 G A); (2257
9776193 C T); (2258 9776270 T A); (2259 9776467 A C); (2260 9776533 A G); (2261 9776549 C T); (2262
9776694 G A); (2263 9776750 A C); (2264 9776864 C T); (2265 9777148 C T); (2266 9777157 G A); (2267
9777176 C T); (2268 9777254 G A); (2269 9777313 G A); (2270 9777474 C A); (2271 9777503 T C); (2272
9777725 C T); (2273 9777767 C T); (2274 9777781 G C); (2275 9777900 T A); (2276 9777901 A G); (2277
9778137 T C); (2278 9778198 A G); (2279 9778219 C T); (2280 9778514 C T); (2281 9778585 A G); (2282
9778641 G A); (2283 9778708 T C); (2284 9778824 A G); (2285 9778965 C T); (2286 9779007 G A); (2287
9779460 C A); (2288 9779492 G T); (2289 9779508 A G); (2290 9779562 A G); (2291 9780627 T C); (2292
9780892 T C); (2293 9780968 A G); (2294 9781049 C T); (2295 9781056 C G); (2296 9781217 T C); (2297
9781251 A G); (2298 9781262 G A); (2299 9781304 C T); (2300 9781334 C T); (2301 9781522 C T); (2302
9781653 T C); (2303 9781918 C T); (2304 9781936 G T); (2305 9782081 G A); (2306 9782359 T C); (2307
9782398 A G); (2308 9782604 A T); (2309 9782647 C A); (2310 9782651 C T); (2311 9782774 T C); (2312
9782828 C T); (2313 9786035 T C); (2314 9786079 T A); (2315 9786151 T C); (2316 9786205 C T); (2317
9786299 T C); (2318 9786349 CT C); (2319 9786548 A G); (2320 9786972 C G); (2321 9787053 C T); (2322
9787450 G A); (2323 9787587 A C); (2324 9787603 G C); (2325 9787640 A G); (2326 9787826 T C); (2327
9787879 C CA); (2328 9788105 C T); (2329 9788257 A G); (2330 9788358 C T); (2331 9788404 A G); (2332
9788426 A T); (2333 9788462 C A); (2334 9788688 T C); (2335 9788964 G A); (2336 9789035 C T); (2337
9789443 T C); (2338 9789484 C T); (2339 9789560 A G); (2340 9789638 G T); (2341 9790130 G T); (2342
9790195 A G); (2343 9790197 C T); (2344 9790731 A G); (2345 9790749 C A); (2346 9790806 C A); (2347
9790843 T C); (2348 9790873 G A); (2349 9790947 C T); (2350 9791258 G T); (2351 9791523 A G); (2352
9791730 G T); (2353 9791914 A G); (2354 9791973 G A); (2355 9792168 T C); (2356 9792267 A G); (2357
9792369 T C); (2358 9792436 T C); (2359 9792463 T C); (2360 9792598 C T); (2361 9792878 A C); (2362
9793343 C T); (2363 9793569 A C); (2364 9793647 G A); (2365 9793660 C A); (2366 9794277 T C); (2367
9794279 G A); (2368 9794343 A G); (2369 9794394 C A); (2370 9794873 T C); (2371 9794983 A T); (2372
9795235 G A); (2373 9795259 T TCACACATTTTGAA); (2374 9795429 G A); (2375 9795779 T C); (2376
9795820 G A); (2377 9796041 C T); (2378 9796079 GA G); (2379 9796155 C T); (2380 9796449 C T); (2381
9796897 C T); (2382 9797014 G C); (2383 9797267 A G); (2384 9797558 C A); (2385 9798278 G A); (2386
9798439 G T); (2387 9798460 T A); (2388 9798562 T G); (2389 9798815 G C); (2390 9798947 A G); (2391
9799007 A G); (2392 9799722 G A); (2393 9799784 G A); (2394 9800298 A C); (2395 9800342 T G); (2396
9800345 T C); (2397 9800424 T A); (2398 9800425 T G); (2399 9800494 C T); (2400 9800810 C T); (2401
9800886 A G); (2402 9800976 A G); (2403 9800977 T C); (2404 9801035 T C); (2405 9801085 G T); (2406
9801115 A G); (2407 9801171 G A); (2408 9801946 A G); (2409 9802093 C G); (2410 9802244 A T); (2411
9802544 G C); (2412 9802734 A C); (2413 9802793 C A); (2414 9802800 T A); (2415 9803157 C T); (2416
9803272 C T); (2417 9803377 C T); (2418 9803409 T C); (2419 9803766 AG A); (2420 9804039 T C); (2421
9805156 G A); (2422 9805186 T A); (2423 9805349 G A); (2424 9805532 AG A); (2425 9805717 G
A); (2426 9805990 C T); (2427 9806164 G A); (2428 9806265 G A); (2429 9806325 G A); (2430 9806392 C
T); (2431 9806488 C T); (2432 9806867 A C); (2433 9806921 G A); (2434 9806993 A G); (2435 9807015 C
T); (2436 9807197 C T); (2437 9807283 T C); (2438 9807521 T C); (2439 9807626 G A); (2440 9807672 G
T); (2441 9807815 T C); (2442 9808296 T C); (2443 9808551 A G); (2444 9808558 C G); (2445 9808569 G
T); (2446 9808591 G A); (2447 9808608 C G); (2448 9808630 C T); (2449 9808665 C T); (2450 9808763 A
G); (2451 9808901 T C); (2452 9809064 G A); (2453 9809248 G A); (2454 9809278 T C); (2455 9809309 T
G); (2456 9809310 T G); (2457 9809326 A G); (2458 9809360 G T); (2459 9809377 T G); (2460 9809459 A
C); (2461 9809588 A C); (2462 9809846 TC T); (2463 9810095 A G); (2464 9810216 CTCAA C); (2465
9810244 C T); (2466 9810624 A G); (2467 9810812 A T); (2468 9810819 G T); (2469 9810948 C
CAT); (2470 9811066 C G); (2471 9811146 GAGCAA G); (2472 9811262 T C); (2473 9811619 A C); (2474
9812024 T C); (2475 9812132 A C); (2476 9812225 A C); (2477 9812432 C T); (2478 9812458 T A); (2479
9812488 T C); (2480 9812518 C A); (2481 9812626 A G); (2482 9812793 G A); (2483 9812956 C T); (2484
9813049 G A); (2485 9813090 C T); (2486 9813109 G A); (2487 9813762 T C); (2488 9813848 G A); (2489
9813867 G A); (2490 9813926 T A); (2491 9814081 G A); (2492 9814119 G A); (2493 9814709 C T); (2494
9814817 A G); (2495 9814885 A G); (2496 9814919 C T); (2497 9815550 G A); (2498 9815766 G A); (2499
9815823 G A); (2500 9816281 C T); (2501 9816414 T A); (2502 9816608 T C); (2503 9816675 A C); (2504
9816729 A G); (2505 9817293 A C); (2506 9817356 A C); (2507 9817433 A G); (2508 9817751 C A); (2509
9818577 G A); (2510 9819066 C T); (2511 9819582 C T); (2512 9819583 A G); (2513 9819975 C T); (2514
9820060 A G); (2515 9820599 T G); (2516 9820741 C A); (2517 9821553 T G); (2518 9821879 A G); (2519
9822550 A G); (2520 9822633 T C); (2521 9822875 A T); (2522 9823488 C T); (2523 9823582 T A); (2524
9823590 C A); (2525 9824288 G A); (2526 9824379 G A); (2527 9824435 G A); (2528 9824636 A G); (2529
9824645 A G); (2530 9824723 A G); (2531 9824814 G A); (2532 9825287 G A); (2533 9825885 T C); (2534
9826382 A G); (2535 9826564 A C); (2536 9826593 G A); (2537 9826605 G C); (2538 9826667 C A); (2539
9826693 A C); (2540 9826716 A T); (2541 9826759 A T); (2542 9826786 A G); (2543 9826826 G A); (2544
9826868 G C); (2545 9826896 T G); (2546 9826900 G A); (2547 9826931 G T); (2548 9826937 C A); (2549
9826979 C T); (2550 9827072 A G); (2551 9827074 A G); (2552 9827132 A G); (2553 9827182 G A); (2554
9827278 C T); (2555 9827398 A G); (2556 9827400 T C); (2557 9827417 A C); (2558 9827439 A G); (2559
9827473 T C); (2560 9827475 T C); (2561 9827719 G A); (2562 9827807 T C); (2563 9827870 T C); (2564
9827986 G T); (2565 9828061 G A); (2566 9828570 G A); (2567 9828736 G A); (2568 9829045 C T); (2569
9829098 G A); (2570 9829370 T A); (2571 9829373 G T); (2572 9829619 TAAA T); (2573 9829800 G
A); (2574 9829840 G T); (2575 9829984 C T); (2576 9830084 C T); (2577 9830433 A C); (2578 9830508 A
G); (2579 9830891 T C); (2580 9830943 T A); (2581 9830998 A G); (2582 9831089 T G); (2583 9831454 G
A); (2584 9831681 T C); (2585 9831734 C T); (2586 9831767 T C); (2587 9831805 T C); (2588 9831902 T
C); (2589 9832129 C G); (2590 9832601 G A); (2591 9832625 A G); (2592 9832834 C A); (2593 9832984 T
C); (2594 9833602 G T); (2595 9835992 G GGAAGAA); (2596 9837562 G A); (2597 9838005 G A); (2598
9839875 G A); (2599 9839878 C A); (2600 9839888 C T); (2601 9839962 C T); (2602 9840159 T G); (2603
9841692 G T); (2604 9841857 C T); (2605 9842190 G A); (2606 9843187 A G); (2607 9844211 C T); (2608
9844220 T C); (2609 9844739 G T); (2610 9845179 A T); (2611 9845319 A G); (2612 9845375 G C); (2613
9845406 G T); (2614 9845453 C T); (2615 9845553 G A); (2616 9845578 G A); (2617 9845589 T C); (2618
9845595 T C); (2619 9845596 A G); (2620 9845603 C A); (2621 9845686 C T); (2622 9845716 C T); (2623
9845725 G A); (2624 9846033 A G); (2625 9846051 G A); (2626 9846133 T G); (2627 9846193 T A); (2628
9846463 T TAA); (2629 9846547 A T); (2630 9846809 C T); (2631 9847168 G A); (2632 9847275 A
T); (2633 9847581 A G); (2634 9847791 T C); (2635 9848747 C T); (2636 9848905 T C); (2637 9849044 C
T); (2638 9849048 C T); (2639 9849090 C T); (2640 9849154 T A); (2641 9849155 T A); (2642 9849195 G
A); (2643 9849430 A G); (2644 9849531 C G); (2645 9850344 T C); (2646 9850431 A G); (2647 9850638
AT A); (2648 9850709 C G); (2649 9850832 C T); (2650 9850843 C T); (2651 9850899 T C); (2652 9850949
G A); (2653 9851121 T C); (2654 9851122 G A); (2655 9851130 C T); (2656 9851304 C A); (2657 9851357
G A); (2658 9851443 C A); (2659 9851460 A G); (2660 9851492 A G); (2661 9851523 G A); (2662 9851541
C G); (2663 9851605 A G); (2664 9851696 A G); (2665 9851761 C T); (2666 9852000 C T); (2667 9852011
G A); (2668 9852049 C T); (2669 9852068 C T); (2670 9852093 T C); (2671 9852487 T A); (2672 9852905 C
T); (2673 9852956 C A); (2674 9852998 T C); (2675 9853007 G A); (2676 9853070 G A); (2677 9853107 T
TGGCGCTGGA); (2678 9853108 C T); (2679 9853123 T C); (2680 9853205 T C); (2681 9853208 C A); (2682
9853231 A G); (2683 9853237 G A); (2684 9853239 T C); (2685 9853334 G T); (2686 9853397 A G); (2687
9853404 C T); (2688 9853408 C T); (2689 9853736 G A); (2690 9854036 A C); (2691 9854055 C T); (2692
9854327 C T); (2693 9854441 C T); (2694 9854816 T C); (2695 9854885 G A); (2696 9854914 T C); (2697
9854964 T C); (2698 9854979 C CG); (2699 9855002 T C); (2700 9855169 T A); (2701 9855239 G A); (2702
9855272 C T); (2703 9855374 C T); (2704 9855403 G A); (2705 9855824 A G); (2706 9855975 C T); (2707
9856479 C T); (2708 9856494 G A); (2709 9856627 C T); (2710 9856666 A T); (2711 9856709 A C); (2712
9857102 C T); (2713 9857288 T C); (2714 9857825 A C); (2715 9857941 A G); (2716 9858163 T C); (2717
9858710 T G); (2718 9858902 G T); (2719 9859326 T A); (2720 9859552 C CA); (2721 9859610 G A); (2722
9859641 G A); (2723 9859871 C A); (2724 9860012 G A); (2725 9860109 T C); (2726 9860142 A G); (2727
9860636 C T); (2728 9860685 T C); (2729 9860896 C A); (2730 9860933 T G); (2731 9860956 A G); (2732
9860998 A G); (2733 9861062 G T); (2734 9861165 T C); (2735 9861300 G C); (2736 9861447 A G); (2737
9861497 T C); (2738 9861505 C T); (2739 9861570 T C); (2740 9861586 G A); (2741 9861653 T C); (2742
9861849 T C); (2743 9861954 T C); (2744 9862004 G A); (2745 9862031 T C); (2746 9862072 G A); (2747
9862181 G A); (2748 9862191 G A); (2749 9862239 G GT); (2750 9862335 C T); (2751 9862618 T C); (2752
9862814 T C); (2753 9862855 T C); (2754 9863164 G C); (2755 9863286 T C); (2756 9863391 G C); (2757
9863543 AAGAGCT A); (2758 9863794 G A); (2759 9863821 G C); (2760 9864139 T C); (2761 9864174 A
G); (2762 9864208 T C); (2763 9864233 C T); (2764 9864533 AT A); (2765 9864628 T C); (2766 9864967 C
T); (2767 9864978 GA G); (2768 9865086 A G); (2769 9865198 C T); (2770 9865203 T C); (2771 9865337 C
T); (2772 9865340 G A); (2773 9865349 A C); (2774 9865419 C T); (2775 9865437 C T); (2776 9865459 T
C); (2777 9865465 T A); (2778 9865469 T C); (2779 9865563 T C); (2780 9865623 GA G); (2781 9865658 T
TA); (2782 9866004 A T); (2783 9866081 T G); (2784 9866292 T TA); (2785 9866808 A G); (2786 9867099
G A); (2787 9867446 CA C); (2788 9868338 G A); (2789 9868372 TTAATACAA T); (2790 9868487 C
A); (2791 9868591 C T); (2792 9868790 A T); (2793 9868987 C A); (2794 9869259 T A); (2795 9869261 AT
A); (2796 9869505 A C); (2797 9869545 T G); (2798 9869573 G T); (2799 9869769 C A); (2800 9869936 T
C); (2801 9869999 A AT); (2802 9870073 GT G); (2803 9870188 C A); (2804 9870204 T C); (2805 9870277
A G); (2806 9870279 G C); (2807 9870381 C G); (2808 9870455 A T); (2809 9870461 A T); (2810 9870578
T C); (2811 9870795 C A); (2812 9870928 C T); (2813 9870996 A G); (2814 9871299 T A); (2815 9871451 A
T); (2816 9871506 G A); (2817 9871613 T C); (2818 9871669 G A); (2819 9871792 T C); (2820 9871818 A
G); (2821 9871953 A C); (2822 9872014 G A); (2823 9872030 T G); (2824 9872160 T TA); (2825 9872223 A
G); (2826 9872291 A G); (2827 9872325 G A); (2828 9872367 A G); (2829 9872415 C T); (2830 9872444 C
A); (2831 9872539 T C); (2832 9872585 T C); (2833 9872641 A G); (2834 9872757 G A); (2835 9872775 G
A); (2836 9873097 A G); (2837 9873519 T C); (2838 9873562 C T); (2839 9873602 A G); (2840 9873778 G
A); (2841 9874260 G A); (2842 9874350 C T); (2843 9874471 G A); (2844 9874503 A G); (2845 9874520 A
G); (2846 9874652 G A); (2847 9874677 A G); (2848 9874685 T C); (2849 9874699 C T); (2850 9874792 A
G); (2851 9874802 C T); (2852 9874820 T C); (2853 9874889 T C); (2854 9875045 A G); (2855 9875048 T
C); (2856 9875107 A G); (2857 9875359 A G); (2858 9875470 A G); (2859 9875477 TA T); (2860 9875550
G A); (2861 9875600 G T); (2862 9875606 T C); (2863 9875685 A C); (2864 9875753 C T); (2865 9876086
C T); (2866 9876189 A G); (2867 9876199 A G); (2868 9876211 T C); (2869 9876256 C T); (2870 9876451
A G); (2871 9876555 A G); (2872 9876568 A AT); (2873 9876703 T C); (2874 9876726 G A); (2875
9876737 A G); (2876 9876803 A G); (2877 9876886 G A); (2878 9876999 C T); (2879 9877028 C T); (2880
9877103 A T); (2881 9877298 C T); (2882 9877327 A G); (2883 9877429 G C); (2884 9877431 T C); (2885
9877435 A G); (2886 9877446 A G); (2887 9877552 T C); (2888 9877570 G A); (2889 9877969 G T); (2890
9878023 C T); (2891 9878081 C T); (2892 9878082 A G); (2893 9878246 G A); (2894 9878263 T C); (2895
9878351 A G); (2896 9878364 T A); (2897 9878665 A G); (2898 9878859 A C); (2899 9878916 T C); (2900
9879047 T C); (2901 9879061 G A); (2902 9879138 A G); (2903 9879139 A T); (2904 9879232 T A); (2905
9879438 C T); (2906 9879450 T C); (2907 9879470 T G); (2908 9879482 A G); (2909 9879484 C T); (2910
9879496 A G); (2911 9879572 T C); (2912 9879979 T C); (2913 9880477 G A); (2914 9880601 T C); (2915
9880685 C T); (2916 9880695 C T); (2917 9880737 A G); (2918 9880747 T C); (2919 9880830 T G); (2920
9880910 A G); (2921 9880927 T G); (2922 9880943 G T); (2923 9881144 ATT A); (2924 9881653 T
C); (2925 9881745 A T); (2926 9881791 T C); (2927 9881821 T C); (2928 9882564 A G); (2929 9882658 A
G); (2930 9882741 G A); (2931 9882755 G T); (2932 9882898 T C); (2933 9883078 C T); (2934 9883089 CT
C); (2935 9883526 A G); (2936 9883661 A G); (2937 9883784 T C); (2938 9883789 G C); (2939 9883803 A
G); (2940 9884181 G A); (2941 9884183 A C); (2942 9884822 G T); (2943 9884831 T C); (2944 9884832 A
G); (2945 9885028 G A); (2946 9885326 G A); (2947 9885338 A G); (2948 9885519 G A); (2949 9885598 G
T); (2950 9885742 A G); (2951 9885860 T C); (2952 9885881 A C); (2953 9885898 G C); (2954 9885920 C
T); (2955 9885938 A G); (2956 9885950 C T); (2957 9886223 T C); (2958 9886450 T C); (2959 9886606 A
G); (2960 9886788 A T); (2961 9887193 A G); (2962 9887669 T G); (2963 9887897 A G); (2964 9888383 T
G); (2965 9888529 C T); (2966 9888530 G A); (2967 9888572 A G); (2968 9888897 G A); (2969 9888936 T
C); (2970 9889109 A C); (2971 9889680 A G); (2972 9889836 T C); (2973 9889906 G A); (2974 9890409 A
G); (2975 9890795 C T); (2976 9890856 C T); (2977 9890881 T C); (2978 9890892 G A); (2979 9890976 T
C); (2980 9891033 A G); (2981 9891118 T C); (2982 9891201 T C); (2983 9891279 G A); (2984 9891424 G
T); (2985 9891484 T C); (2986 9891498 G A); (2987 9891528 C T); (2988 9891590 C T); (2989 9891936 G
A); (2990 9891960 C T); (2991 9891995 G A); (2992 9892173 G A); (2993 9892236 A G); (2994 9892521 G
A); (2995 9892553 A G); (2996 9892581 C T); (2997 9892592 T C); (2998 9892629 G A); (2999 9892763 T
C); (3000 9892787 C T); (3001 9893054 G A); (3002 9893065 T C); (3003 9893108 A G); (3004 9893132 G
A); (3005 9893161 T C); (3006 9893239 A G); (3007 9893272 G A); (3008 9893283 G T); (3009 9893327 T
G); (3010 9893359 G A); (3011 9893457 C A); (3012 9893732 A G); (3013 9893972 G A); (3014 9894411 A
G); (3015 9894619 A G); (3016 9895052 C T); (3017 9896333 G A); (3018 9897167 C T); (3019 9897254 C
A); (3020 9897866 A G); (3021 9898843 A C); (3022 9898926 T TAC); (3023 9898933 C A); (3024 9899029
A C); (3025 9899056 C T); (3026 9899064 G T); (3027 9899076 A G); (3028 9899135 C T); (3029 9899161 T
C); (3030 9899162 A G); (3031 9899172 A G); (3032 9900237 A G); (3033 9900282 T C); (3034 9900387 T
C); (3035 9900931 T A); (3036 9901491 T C); (3037 9901492 G A); (3038 9901519 T C); (3039 9901560 A
G); (3040 9901759 T C); (3041 9901823 A G); (3042 9902035 T C); (3043 9902136 T C); (3044 9902152 T
TACGA); (3045 9902241 G A); (3046 9902373 T A); (3047 9902388 TA T); (3048 9902412 T G); (3049
9902731 C G); (3050 9903060 G GA); (3051 9903113 C T); (3052 9903289 A G); (3053 9903296 G
T); (3054 9903390 C A); (3055 9903475 C A); (3056 9903479 GA G); (3057 9903587 G A); (3058 9903618
G A); (3059 9904003 A G); (3060 9904255 CA C); (3061 9904289 TTTAA T); (3062 9904370 A AC); (3063
9904583 A C); (3064 9905036 C T); (3065 9905190 T C); (3066 9905530 G A); (3067 9905558 T
TGTTTTTGAA); (3068 9905571 T G); (3069 9905725 T G); (3070 9905799 G A); (3071 9905809 A G); (3072
9905883 G A); (3073 9905931 C T); (3074 9906021 G A); (3075 9906084 A G); (3076 9906100 C A); (3077
9906414 G A); (3078 9906654 T C); (3079 9906669 TA T); (3080 9906944 C T); (3081 9906975 G A); (3082
9907517 A G); (3083 9907562 A C); (3084 9907627 C T); (3085 9907725 T C); (3086 9907811 AC A); (3087
9907875 A G); (3088 9908249 G A); (3089 9908265 T G); (3090 9908315 A G); (3091 9908692 A T); (3092
9908722 T C); (3093 9908771 G A); (3094 9908815 C T); (3095 9908933 C T); (3096 9909070 T C); (3097
9909099 C T); (3098 9909227 A G); (3099 9909339 G C); (3100 9909366 GT G); (3101 9909387 G
A); (3102 9910286 G C); (3103 9910423 C T); (3104 9910499 T C); (3105 9910665 G A); (3106 9910696 GT
G); (3107 9910776 T C); (3108 9910789 A G); (3109 9910821 T G); (3110 9911261 G A); (3111 9911515 A
C); (3112 9912066 C CT); (3113 9912105 T C); (3114 9912455 A G); (3115 9912486 A G); (3116 9912505 G
A); (3117 9912511 A G); (3118 9912554 T C); (3119 9912603 A G); (3120 9912680 C T); (3121 9912698 T
C); (3122 9912748 G C); (3123 9912788 C T); (3124 9912847 A G); (3125 9912959 C T); (3126 9913278 A
G); (3127 9913306 T C); (3128 9913476 A G); (3129 9913737 A G); (3130 9913784 A G); (3131 9914143 A
G); (3132 9914179 A G); (3133 9914283 G A); (3134 9914288 A G); (3135 9914630 A G); (3136 9914706 C
T); (3137 9914715 C T); (3138 9914723 G A); (3139 9914764 T C); (3140 9914843 T G); (3141 9914865 A
G); (3142 9914988 T C); (3143 9915110 T A); (3144 9915477 G A); (3145 9915480 T G); (3146 9915485 T
C); (3147 9915699 G T); (3148 9916666 A G); (3149 9916668 C T); (3150 9917066 C T); (3151 9917206 A
C); (3152 9917348 G A); (3153 9917355 G T); (3154 9917369 G A); (3155 9917655 G A); (3156 9917762 T
A); (3157 9917879 A G); (3158 9917933 C A); (3159 9918216 T C); (3160 9918725 G A); (3161 9918902 T
C); (3162 9918935 G A); (3163 9918994 T C); (3164 9919069 A G); (3165 9919079 A G); (3166 9919089 T
C); (3167 9919115 G T); (3168 9919123 A G); (3169 9919138 T C); (3170 9919157 A T); (3171 9919158 T
G); (3172 9919192 A C); (3173 9919269 C T); (3174 9919506 T G); (3175 9919564 C A); (3176 9919591 C
T); (3177 9920109 T C); (3178 9920590 A G); (3179 9920698 A G); (3180 9920834 C T); (3181 9921059 T
C); (3182 9921079 G A); (3183 9921097 T C); (3184 9921115 G A); (3185 9921226 T C); (3186 9921275 A
G); (3187 9921402 A G); (3188 9921421 A G); (3189 9921698 T C); (3190 9921788 T C); (3191 9921854 G
A); (3192 9921975 T G); (3193 9922069 T C); (3194 9922139 T C); (3195 9922394 A C); (3196 9922457 T
C); (3197 9922525 T C); (3198 9922717 G A); (3199 9922908 T C); (3200 9922962 A G); (3201 9923309 A
T); (3202 9923463 G A); (3203 9924577 G A); (3204 9925033 T C); (3205 9925524 C T); (3206 9925625 G
C); (3207 9926198 G A); (3208 9926476 C T); (3209 9926600 T C); (3210 9926946 A G); (3211 9927015 T
C); (3212 9927031 T G); (3213 9927175 T C); (3214 9927387 T C); (3215 9927435 A G); (3216 9927724 G
A); (3217 9927981 T A); (3218 9928077 T C); (3219 9928123 G A); (3220 9928152 A C); (3221 9928153 G
A); (3222 9928154 T C); (3223 9928155 G T); (3224 9928317 A C); (3225 9928325 C T); (3226 9928657 C
T); (3227 9928750 C T); (3228 9928858 A G); (3229 9928955 C A); (3230 9928974 A C); (3231 9929191 A
ATTACTATTATTATTATTATTATTG); (3232 9929240 T C); (3233 9929274 C T); (3234 9929420 A G); (3235
9929491 A G); (3236 9929508 ACCC A); (3237 9929757 C G); (3238 9929791 G A); (3239 9929862 G
A); (3240 9930019 A G); (3241 9930054 AT A); (3242 9930100 T A); (3243 9930119 C T); (3244 9930124 A
G); (3245 9930125 G C); (3246 9930126 C T); (3247 9930189 A G); (3248 9930213 A T); (3249 9930245 G
A); (3250 9930273 GA G); (3251 9930503 A G); (3252 9930595 T C); (3253 9931046 T C); (3254 9931102 T
G); (3255 9931390 A G); (3256 9931401 T C); (3257 9931433 C A); (3258 9931437 G A); (3259 9931460 T
C); (3260 9931761 A G); (3261 9931871 T C); (3262 9932054 T C); (3263 9932592 C T); (3264 9932719 G
A); (3265 9932738 C T); (3266 9932815 T A); (3267 9933791 A C); (3268 9933816 G T); (3269 9933991 A
G); (3270 9936932 T C); (3271 9937601 G A); (3272 9937905 T A); (3273 9938288 G A); (3274 9938493 C
G); (3275 9938553 T C); (3276 9939296 C A); (3277 9939396 A G); (3278 9939638 T C); (3279 9939650 G
A); (3280 9939678 C A); (3281 9940006 C A); (3282 9940114 C T); (3283 9940171 A C); (3284 9940211 A
T); (3285 9940319 G A); (3286 9940325 A G); (3287 9940327 T G); (3288 9940413 T C); (3289 9940420 A
G); (3290 9940481 T A); (3291 9940587 T C); (3292 9941272 C T); (3293 9941367 A G); (3294 9941776 A
C); (3295 9942111 T C); (3296 9942219 A G); (3297 9942404 A G); (3298 9943103 G A); (3299 9943180 C
T); (3300 9943239 A G); (3301 9943449 A T); (3302 9943615 A C); (3303 9943618 G A); (3304 9944209 G
A); (3305 9944334 G A); (3306 9944396 T C); (3307 9944408 C G); (3308 9944494 C T); (3309 9944960 T
C); (3310 9945158 T C); (3311 9945623 G A); (3312 9945742 A G); (3313 9945758 A G); (3314 9945774
TTG T); (3315 9945807 G A); (3316 9946109 A G); (3317 9946207 A T); (3318 9946246 T A); (3319
9946328 G A); (3320 9946332 A T); (3321 9946402 G A); (3322 9946409 C T); (3323 9946774 T G); (3324
9946980 C T); (3325 9947270 G C); (3326 9947455 T A); (3327 9947465 A G); (3328 9947528 A G); (3329
9947582 CA C); (3330 9948002 T G); (3331 9948235 CT C); (3332 9948816 C T); (3333 9949058 A
G); (3334 9949324 C T); (3335 9950465 A G); (3336 9950682 G T); (3337 9950858 C T); (3338 9951041 T
C); (3339 9951374 T C); (3340 9951588 C T); (3341 9951619 A G); (3342 9951641 T G); (3343 9952034 T
C); (3344 9952244 G A); (3345 9952472 G C); (3346 9953202 C T); (3347 9953228 A G); (3348 9953346 C
T); (3349 9953356 G A); (3350 9953386 C T); (3351 9953709 T TG); (3352 9954093 C A); (3353 9954174 C
T); (3354 9954191 T C); (3355 9955211 G T); (3356 9960684 CACAACAG C); (3357 9961405 A G); (3358
9961697 C T); (3359 9961932 A AT); (3360 9961993 A G); (3361 9962057 A T); (3362 9962637 T A); (3363
9963642 G A); (3364 9963980 G T); (3365 9964360 AT A); (3366 9964369 C T); (3367 9964550 G A); (3368
9964595 T C); (3369 9964712 A C); (3370 9964741 A G); (3371 9965412 A G); (3372 9965530 G A); (3373
9965571 T TA); (3374 9965713 T A); (3375 9965849 T G); (3376 9966113 A AT); (3377 9966890 G
T); (3378 9967200 G A); (3379 9967519 G A); (3380 9967528 A AAGGTG); (3381 9968097 G C); (3382
9968304 A G); (3383 9968808 A AT); (3384 9969430 G C); (3385 9969527 T TA); (3386 9973835 T
A); (3387 9974181 A G); (3388 9974474 C T); (3389 9974770 C T); (3390 9975102 AT A); (3391 9975289 G
A); (3392 9975478 C T); (3393 9975528 G A); (3394 9975784 C T); (3395 9976194 C T); (3396 9976237 C
T); (3397 9976253 GCAACTTTTGGCAACA G); (3398 9976347 T G); (3399 9977196 G A); (3400 9978449 G
A); (3401 9979232 A C); (3402 9979377 C T); (3403 9979821 C G); (3404 9980013 A T); (3405 9980369 A
T); (3406 9980854 C T); (3407 9981001 C T); (3408 9981095 G A); (3409 9981139 A G); (3410 9981251 A
G); (3411 9981781 C T); (3412 9982469 A G); (3413 9982606 C G); (3414 9983560 G C); (3415 9983591 G
A); (3416 9984336 A G); (3417 9984639 C T); (3418 9985324 C T); (3419 9986240 T C); (3420 9986710 C
T); (3421 9987822 G A); (3422 9987863 T A); (3423 9987883 A G); (3424 9988484 A G); (3425 9988993 G
A); (3426 9989478 T C); (3427 9990917 C T); (3428 9991022 C A); (3429 9991164 A AG); (3430 9991206 A
G); (3431 9991254 A G); (3432 9991328 T C); (3433 9991938 T C); (3434 9992163 G A); (3435 9993582 T
C); (3436 9993843 C T); (3437 9994102 G A); (3438 10005182 G A); (3439 10005221 G A); (3440
10005953 A G); (3441 10006306 A G); (3442 10006308 C T); (3443 10006468 G A); (3444 10006765 G
T); (3445 10007105 A G); (3446 10007888 C A); (3447 10008071 G T); (3448 10008094 G A); (3449
10008238 T C); (3450 10009156 G A); (3451 10010904 A G); (3452 10011566 A G); (3453 10011574 G
T); (3454 10011634 A G); (3455 10012253 T C); (3456 10012341 G A); (3457 10012386 A G); (3458
10013958 A T); (3459 10014019 G A); (3460 10015872 C A); (3461 10016751 A G); (3462 10016879 C
T); (3463 10016883 A G); (3464 10017093 T C); (3465 10017166 A G); (3466 10017217 G A); (3467
10017253 T C); (3468 10017616 G GT); (3469 10018002 G A); (3470 10018081 G A); (3471 10018452 T
C); (3472 10018461 A G); (3473 10018539 A G); (3474 10018545 G T); (3475 10018551 G A); (3476
10018581 C T); (3477 10018590 C T); (3478 10018828 A G); (3479 10019076 G A); (3480 10019111 T
C); (3481 10020301 T C); (3482 10020407 AGC A); (3483 10020700 C T); (3484 10021186 C T); (3485
10021187 A G); (3486 10021203 A AT); (3487 10022026 A ATATATATATATG); (3488 10022082 A
T); (3489 10022124 G C); (3490 10022967 T C); (3491 10023821 G T); (3492 10024331 T C); (3493
10024489 A G); (3494 10024510 A C); (3495 10025699 T C); (3496 10026639 T G); (3497 10026770 C
T); (3498 10027551 G C); (3499 10028076 T A); (3500 10028082 C T); (3501 10028290 T C); (3502
10029886 C T); (3503 10030409 T A); (3504 10030748 C T); (3505 10030850 G A); (3506 10030946 C
T); (3507 10031562 A T); (3508 10031826 C T); (3509 10031859 T G); (3510 10032867 A G); (3511
10033750 A C); (3512 10033756 A G); (3513 10034895 G T); (3514 10034926 C T); (3515 10035682 A
G); (3516 10036079 A G); (3517 10036267 A G); (3518 10036269 G A); (3519 10036283 C T); (3520
10036284 A G); (3521 10036379 G A); (3522 10036442 T C); (3523 10037260 TA T); (3524 10037382 T
TA); (3525 10037881 A T); (3526 10037891 T A); (3527 10038900 T C); (3528 10040185 C A); (3529
10040535 A C); (3530 10040806 A G); (3531 10040991 C G); (3532 10042843 T C); (3533 10043955 CT
C); (3534 10044537 C T); (3535 10045127 AT A); (3536 10045162 T TA); (3537 10045290 G A); (3538
10045361 G T); (3539 10045739 C T); (3540 10046273 C T); (3541 10046413 G A); (3542 10046416 G
A); (3543 10046421 C T); (3544 10046903 T C); (3545 10046940 A G); (3546 10047874 C T); (3547
10049142 C T); (3548 10050487 C T); (3549 10050608 T C); (3550 10051024 C T); (3551 10051175 G
T); (3552 10051393 T G); (3553 10051871 C T); (3554 10052086 A G); (3555 10052878 T A); (3556
10054531 G A); (3557 10055765 C CA); (3558 10056012 A ATTATCTATCTT); (3559 10056735 G A); (3560
10056912 C A); (3561 10056972 G A); (3562 10058310 A G); (3563 10062027 G A); (3564 10064510 G
T); (3565 10064774 G A); (3566 10065555 A G); (3567 10065568 A T); (3568 10065727 T G); (3569
10065804 T C); (3570 10065887 C T); (3571 10066455 C T); (3572 10067105 C T); (3573 10067146 T
C); (3574 10067826 TA T); (3575 10070284 G T); (3576 10070518 G A); (3577 10070946 C CAA); (3578
10071616 G A); (3579 10071739 T G); (3580 10071864 G A); (3581 10071931 TA T); (3582 10072931 A
G); (3583 10073443 T A); (3584 10073983 G A); (3585 10074721 T C); (3586 10074823 G A); (3587
10077861 CATATATATAT C); (3588 10082486 T C); (3589 10082575 G A); (3590 10083027 G T); (3591
10083901 T C); (3592 10083942 C G); (3593 10084430 C T); (3594 10084921 T C); (3595 10085379 C
A); (3596 10086422 T C); (3597 10087625 G A); (3598 10087655 C T); (3599 10087674 G A); (3600
10087693 C T); (3601 10087759 T C); (3602 10087761 C T); (3603 10090671 A G); (3604 10091409 T
C); (3605 10093615 C T); (3606 10093774 A G); (3607 10093893 T C); (3608 10093908 A T); (3609
10094020 C T); (3610 10094169 C CG); (3611 10094284 CA C); (3612 10094571 C T); (3613 10094854 A
G); (3614 10096086 C T); (3615 10096148 G A); (3616 10097436 C T); (3617 10097513 G C); (3618
10097669 T C); (3619 10097831 T G); (3620 10097983 A T); (3621 10098433 A G); (3622 10098628 T
C); (3623 10098744 T C); (3624 10099010 A G); (3625 10099526 T C); (3626 10099655 T
TTGGATGTG); (3627 10099738 C A); (3628 10099752 T C); (3629 10100358 G A); (3630 10102932 G
A); (3631 10103239 C CTT); (3632 10103286 T C); (3633 10103367 T C); (3634 10103405 T A); (3635
10103407 T A); (3636 10103409 T A); (3637 10103420 C T); (3638 10103978 C T); (3639 10104636 A
C); (3640 10106219 A G); (3641 10107331 C A); (3642 10107740 T G); (3643 10107982 G A); (3644
10108492 A T); (3645 10108590 T C); (3646 10108618 T C); (3647 10108683 C T); (3648 10108929 C
T); (3649 10109447 G A); (3650 10111136 A G); (3651 10111177 A T); (3652 10111307 G A); (3653
10112784 CA C); (3654 10112845 A C); (3655 10113055 TAA T); (3656 10113155 A AAT); (3657
10113206 A G); (3658 10114399 GT G); (3659 10114463 A G); (3660 10114937 C T); (3661 10115688 TA
T); (3662 10116627 T C); (3663 10117060 A G); (3664 10117154 A G); (3665 10118749 A G); (3666
10120512 A G); (3667 10124209 T C); (3668 10126825 C T); (3669 10126998 C CAT); (3670 10128142 G
A); (3671 10128403 A C); (3672 10128514 G A); (3673 10129033 T C); (3674 10129213 C T); (3675
10129263 A G); (3676 10129492 C CTG); (3677 10129837 A AAT); (3678 10131119 C T); (3679 10131417
C T); (3680 10131914 T C); (3681 10131981 C A); (3682 10132576 C G); (3683 10132590 C T); (3684
10133823 G T); (3685 10133915 T TTTTA); (3686 10134043 T C); (3687 10134453 T C); (3688 10134492 G
A); (3689 10134908 GT G); (3690 10135584 T G); (3691 10135617 C CTAGA); (3692 10136188 G A); (3693
10136470 G A); (3694 10137487 T C); (3695 10137617 A G); (3696 10137806 C T); (3697 10138612 G
A); (3698 10138667 G A); (3699 10138774 A T); (3700 10138803 T C); (3701 10139527 TA T); (3702
10139669 AT A); (3703 10140491 A C); (3704 10140759 AAAG A); (3705 10142796 C T); (3706 10142852
C T); (3707 10144731 T C); (3708 10145900 C T); (3709 10146461 A G); (3710 10146508 TATAACC
T); (3711 10146587 C A); (3712 10147277 T C); (3713 10148386 G A); (3714 10149197 G A); (3715
10149910 A C); (3716 10149964 A G); (3717 10150154 A G); (3718 10150301 A C); (3719 10151784 T
C); (3720 10152030 A G); (3721 10152467 A G); (3722 10152728 A T); (3723 10152991 T C); (3724
10153163 G A); (3725 10153766 G T); (3726 10154455 C G); (3727 10155043 GT G); (3728 10155154 C
T); (3729 10155210 G A); (3730 10155337 C T); (3731 10155860 C T); (3732 10156469 G A); (3733
10157183 T A); (3734 10157213 A G); (3735 10157256 T C); (3736 10158699 C G); (3737 10158834 C
A); (3738 10160281 C CA); (3739 10161130 C T); (3740 10161401 T A); (3741 10161976 A G); (3742
10161977 G A); (3743 10163392 C T); (3744 10163449 T G); (3745 10163565 A AT); (3746 10163732 C
A); (3747 10163763 A G); (3748 10164387 A G); (3749 10164451 G A); (3750 10164650 T C); (3751
10164802 G A); (3752 10165422 C G); (3753 10165518 T A); (3754 10165646 G T); (3755 10165686 G
A); (3756 10165731 T C); (3757 10165746 T C); (3758 10166532 G A); (3759 10166559 A G); (3760
10166603 A T); (3761 10166652 G A); (3762 10166764 A C); (3763 10166808 G A); (3764 10167704 T
G); (3765 10168012 G C); (3766 10168017 G GAC); (3767 10170440 C A); (3768 10170829 G T); (3769
10170966 G A); (3770 10171056 T C); (3771 10172344 C T); (3772 10172462 G T); (3773 10172463 G
A); (3774 10173323 C A); (3775 10173333 A G); (3776 10173428 T C); (3777 10173530 T C); (3778
10173927 C T); (3779 10174019 C T); (3780 10175148 C T); (3781 10175188 G A); (3782 10175389 A
G); (3783 10176092 G A); (3784 10176700 C T); (3785 10176981 T C); (3786 10177025 G A); (3787
10177263 T TA); (3788 10177733 G A); (3789 10178559 T TA); (3790 10179678 A G); (3791 10179760 C
T); (3792 10179839 C T); (3793 10179878 A G); (3794 10180791 A G); (3795 10181161 T C); (3796
10182512 G A); (3797 10182785 C T); (3798 10183114 T C); (3799 10183573 A T); (3800 10183801 T
C); (3801 10184480 G A); (3802 10184482 G A); (3803 10186558 C T); (3804 10186634 A G); (3805
10186835 C T); (3806 10187486 G A); (3807 10189978 G A); (3808 10189991 G A); (3809 10191590 C
T); (3810 10191591 G A); (3811 10191967 C T); (3812 10193886 G A); (3813 10195420 A T); (3814
10195421 T A); (3815 10195422 A T); (3816 10195423 T A); (3817 10195424 A T); (3818 10195824 A
T); (3819 10196747 G A); (3820 10196877 C T); (3821 10196889 GA G); (3822 10197525 A C); (3823
10201121 A C); (3824 10202303 C T); (3825 10202410 G A); (3826 10202460 C T); (3827 10203192 A
G); (3828 10203197 A T); (3829 10204559 AT A); (3830 10207533 A T); (3831 10208756 C T); (3832
10215839 GGAC G); (3833 10217630 C CTTTCA); (3834 10217871 G A); (3835 10217913 G A); (3836
10218403 A G); (3837 10218447 A G); (3838 10219191 T G); (3839 10220632 T C); (3840 10220874 T
C); (3841 10222622 G C); (3842 10222632 A G); (3843 10222932 T C); (3844 10223175 G A); (3845
10224976 G A); (3846 10224978 G GTGTTGAATGA); (3847 10225038 A G); (3848 10225146 T C); (3849
10225199 G A); (3850 10225256 T C); (3851 10226166 A T); (3852 10226539 T G); (3853 10226639 A
T); (3854 10226640 T A); (3855 10226686 A G); (3856 10229712 G GAAAAAGAGAAAAA); (3857
10234047 T C); (3858 10234051 AT A); (3859 10234954 T A); (3860 10236973 G A); (3861 10236980 C
A); (3862 10241527 C T); (3863 10243470 T TTTACCCTTCGTGACGCAACATGACTGCTGG); (3864 10245896
G T); (3865 10245943 T C); (3866 10245981 G A); (3867 10246092 G A); (3868 10246484 G A); (3869
10246577 T TA); (3870 10246764 A G); (3871 10246792 A AATT); (3872 10247956 G A); (3873 10247979
G A); (3874 10248291 G A); (3875 10248427 G A); (3876 10248446 T C); (3877 10248462 G A); (3878
10248505 T C); (3879 10248729 A G); (3880 10248751 G A); (3881 10248755 G A); (3882 10248850 C
T); (3883 10249288 G A); (3884 10250487 C T); (3885 10250878 G T); (3886 10251014 C T); (3887
10251050 A G); (3888 10255731 TGCAGTG T); (3889 10262570 A G); (3890 10264058 G A); (3891
10264061 T C); (3892 10265321 G T); (3893 10265322 C T); (3894 10275803 T C); (3895 10277533 G
A); (3896 10279100 C T); (3897 10279311 A G); (3898 10279561 A C); (3899 10280435 A G); (3900
10280535 A T); (3901 10281336 G A); (3902 10281338 T A); (3903 10282419 C T); (3904 10282896 C
A); (3905 10286276 A AG); (3906 10287794 T A); (3907 10292046 G A); (3908 10292842 T C); (3909
10295046 G A); (3910 10301761 G A); (3911 10301786 G A); (3912 10301944 C T); (3913 10302115 T
C); (3914 10307203 G T); (3915 10307804 T C); (3916 10309238 T TA); (3917 10309583 GA G); (3918
10309672 T A); (3919 10311350 AT A); (3920 10312410 AAGAG A); (3921 10313534 C T); (3922
10318478 C T); (3923 10319254 ATGCTG A); (3924 10319594 C G); (3925 10319714 G A); (3926
10319784 T A); (3927 10319944 C T); (3928 10319993 C A); (3929 10320527 G C); (3930 10320713 C
T); (3931 10320733 GA G); (3932 10320742 G A); (3933 10320753 C T); (3934 10321858 T G); (3935
10322852 G A); (3936 10322993 T C); (3937 10323338 GT G); (3938 10325415 T A); (3939 10330889 C
T); (3940 10336113 C T); (3941 10336128 A G); (3942 10336227 C G); (3943 10336317 T A); (3944
10336386 C T); (3945 10336410 G A); (3946 10336880 C T); (3947 10338579 A G); (3948 10342374 G
A); (3949 10342603 G A); (3950 10343503 TA T); (3951 10346041 A G); (3952 10346050 T C); (3953
10347073 C T); (3954 10356845 A AT); (3955 10357853 TAG T); (3956 10358135 T TA); (3957 10358485
TA T); (3958 10359153 GA G); (3959 10361243 T C); (3960 10362127 T C); (3961 10362167 A G); (3962
10363298 C A); (3963 10363626 T C); (3964 10363775 GA G); (3965 10364141 A T); (3966 10364197 C
A); (3967 10364311 T A); (3968 10364312 T A); (3969 10364322 T C); (3970 10364350 A AT); (3971
10364469 C CA); (3972 10364742 T A); (3973 10364974 C T); (3974 10365115 C T); (3975 10365128 C
T); (3976 10365131 G A); (3977 10365325 C T); (3978 10365379 G A); (3979 10365424 GA G); (3980
10365666 GA G); (3981 10368032 G A); (3982 10368392 G A); (3983 10368506 G A); (3984 10368583 A
AT); (3985 10369457 G T); (3986 10371781 A G); (3987 10372421 T G); (3988 10372461 A
AGGAGTTATTTGAGGACAC); (3989 10374192 GA G); (3990 10374706 T A); (3991 10374888 A G); (3992
10375330 T C); (3993 10375417 A C); (3994 10375560 C T); (3995 10375582 A G); (3996 10375954 A
G); (3997 10377099 G A); (3998 10377595 G A); (3999 10377616 T C); (4000 10378803 G A); (4001
10379161 T C); (4002 10379280 A G); (4003 10380788 T C); (4004 10380839 AAG A); (4005 10380994 A
C); (4006 10381347 G A); (4007 10382196 T C); (4008 10382836 G A); (4009 10383977 C G); (4010
10387102 T G); (4011 10389697 G A); (4012 10390928 T A); (4013 10391173 A G); (4014 10391631 T
A); (4015 10391638 T C); (4016 10391848 C A); (4017 10391871 A G); (4018 10392671 C A); (4019
10393413 G A); (4020 10394345 A G); (4021 10394380 T C); (4022 10394437 A G); (4023 10398791 C
T); (4024 10398936 C T); (4025 10400987 A G); (4026 10403943 TG T); (4027 10404593 G A); (4028
10407504 T A); (4029 10408992 A G); (4030 10409134 C T); (4031 10410655 T G); (4032 10412326 G
A); (4033 10415313 T C); (4034 10415583 TA T); (4035 10415651 T C); (4036 10415981 T A); (4037
10416695 T TAAATTTC); (4038 10420121 T G); (4039 10421326 A G); (4040 10421328 T C); (4041
10421390 G A); (4042 10421404 C T); (4043 10421409 G A); (4044 10421424 T C); (4045 10421568 C
T); (4046 10421599 C T); (4047 10422004 G A); (4048 10422902 A G); (4049 10423405 T G); (4050
10423857 T C); (4051 10424291 T C); (4052 10425223 A G); (4053 10425270 C T); (4054 10425701 T
A); (4055 10426081 G A); (4056 10426095 GA G); (4057 10426730 C T); (4058 10428005 C T); (4059
10436122 C G); (4060 10437179 T C); (4061 10437489 C G); (4062 10439272 G T); (4063 10447036 G
A); (4064 10448277 G A); (4065 10448314 T C); (4066 10448534 G A); (4067 10449022 C T); (4068
10451605 A G); (4069 10452273 C A); (4070 10452308 G A); (4071 10453224 C T); (4072 10455720 G
A); (4073 10458452 C T); (4074 10465842 G A); (4075 10465845 C T); (4076 10468301 C T); (4077
10468387 T G); (4078 10468435 C CTT); (4079 10468895 TTGTAA T); (4080 10468983 A G); (4081
10470070 T C); (4082 10470395 CTCGA C); (4083 10470473 A G); (4084 10470562 C A); (4085 10473852
T C); (4086 10474643 T G); (4087 10475559 A T); (4088 10476624 T G); (4089 10476872 T C); (4090
10477881 G A); (4091 10477974 A G); (4092 10477991 G A); (4093 10478812 C T); (4094 10479690 T
C); (4095 10479696 G A); (4096 10479697 A G); (4097 10480301 T C); (4098 10480373 A AT); (4099
10481431 G A); (4100 10482118 T G); (4101 10484270 C G); (4102 10487638 CAT C); (4103 10488243 A
G); (4104 10488249 GA G); (4105 10489061 A G); (4106 10489084 G A); (4107 10489920 C G); (4108
10490478 G T); (4109 10490525 ATGATACTCTGTT A); (4110 10490779 A C); (4111 10491525 T C); (4112
10492176 C T); (4113 10492690 T C); (4114 10493398 C T); (4115 10493656 C T); (4116 10493821 G
A); (4117 10495092 T C); (4118 10495358 A T); (4119 10495359 G A); (4120 10495447 C A); (4121
10495450 C T); (4122 10495456 C T); (4123 10496268 G A); (4124 10496287 A G); (4125 10498456 ATG
A); (4126 10499673 T C); (4127 10500050 C T); (4128 10501627 C T); (4129 10501872 T A); (4130
10502028 C T); (4131 10502137 T C); (4132 10502481 C T); (4133 10502512 A G); (4134 10502513 C
A); (4135 10502625 T C); (4136 10502707 C A); (4137 10503620 G A); (4138 10504017 T A); (4139
10504200 G A); (4140 10504222 G A); (4141 10504293 A C); (4142 10504749 T C); (4143 10505004 A
C); (4144 10505042 G A); (4145 10506427 G A); (4146 10507244 A G); (4147 10509643 A G); (4148
10510969 A C); (4149 10511155 G A); (4150 10512521 A G); (4151 10512583 TC T); (4152 10514341 G
A); (4153 10516281 C T); (4154 10516368 C CT); (4155 10518418 A G); (4156 10520090 T C); (4157
10520151 G GAAATACACTA); (4158 10522081 T C); (4159 10522637 A T); (4160 10523561 C A); (4161
10523610 C T); (4162 10523863 C G); (4163 10524909 C A); (4164 10526655 G T); (4165 10526776 A
G); (4166 10527260 C T); (4167 10527280 A G); (4168 10527378 A T); (4169 10527788 T C); (4170
10528202 C T); (4171 10528440 C CAA); (4172 10528449 G T); (4173 10528839 C T); (4174 10529364 C
T); (4175 10529598 A G); (4176 10529626 A G); (4177 10529971 T C); (4178 10530383 C A); (4179
10531424 T C); (4180 10532056 C T); (4181 10532205 G A); (4182 10532696 A G); (4183 10533153 T
C); (4184 10533220 T A); (4185 10533523 A G); (4186 10533635 C T); (4187 10535678 T C); (4188
10535922 A G); (4189 10536595 C T); (4190 10536638 T A); (4191 10539045 G A); (4192 10539234 C
G); (4193 10539519 A G); (4194 10540128 T C); (4195 10540129 C A); (4196 10540216 A G); (4197
10540723 T A); (4198 10540791 G A); (4199 10543650 A G); (4200 10548064 T C); (4201 10548199 CA
C); (4202 10548231 A T); (4203 10560883 A G); (4204 10561098 A C); (4205 10561256 A G); (4206
10563390 ATATTT A); (4207 10563625 A T); (4208 10565407 T A); (4209 10565497 G A); (4210 10565508
G A); (4211 10566534 TGGAAGAAGAACAAGCCTTATATA T); (4212 10568477 T G); (4213 10570278 A
G); (4214 10570501 T C); (4215 10573953 C T); (4216 10574009 T C); (4217 10574016 G A); (4218
10574236 C T); (4219 10574247 G A); (4220 10574541 G T); (4221 10575782 A AT); (4222 10577624 C
T); (4223 10577767 TAG T); (4224 10578506 G A); (4225 10579114 A G); (4226 10579331 G T); (4227
10582958 C G); (4228 10583112 TAA T); (4229 10584154 ATCAATTTT A); (4230 10584306 G A); (4231
10585267 C A); (4232 10585756 T C); (4233 10585935 A G); (4234 10585991 C T); (4235 10586732 C
A); (4236 10587236 GA G); (4237 10588426 T A); (4238 10588442 AT A); (4239 10588918 GA G); (4240
10590143 AAGGAT A); (4241 10590885 T TA); (4242 10591124 G A); (4243 10591138 G A); (4244
10591439 T C); (4245 10591449 C T); (4246 10591668 C T); (4247 10591680 G A); (4248 10592130 A
G); (4249 10592411 G A); (4250 10592719 T C); (4251 10593470 C A); (4252 10593541 A G); (4253
10593698 TG T); (4254 10594094 T C); (4255 10594120 C T); (4256 10594186 C T); (4257 10594277 G
A); (4258 10594586 A G); (4259 10594926 T C); (4260 10594938 G A); (4261 10597198 A G); (4262
10597919 T C); (4263 10598033 T A); (4264 10598144 T C); (4265 10598204 A G); (4266 10598577 C
A); (4267 10599578 GAGA G); (4268 10599678 C T); (4269 10599680 T C); (4270 10599819 C T); (4271
10600865 C T); (4272 10600993 A G); (4273 10602635 G A); (4274 10603936 A G); (4275 10604109 A
AATATATATATAT); (4276 10604359 G A); (4277 10604673 T C); (4278 10608145 A T); (4279 10609909 G
A); (4280 10610607 C T); (4281 10610979 C T); (4282 10611767 A G); (4283 10612518 T C); (4284
10613759 A G); (4285 10614410 GA G); (4286 10614830 T A); (4287 10616025 C T); (4288 10616068 G
T); (4289 10616121 T A); (4290 10616220 A C); (4291 10616294 G A); (4292 10616431 C A); (4293
10616941 C T); (4294 10617879 T A); (4295 10617945 A G); (4296 10619714 A C); (4297 10622675 A
AT); (4298 10623091 A C); (4299 10624032 A G); (4300 10624069 T C); (4301 10624083 G A); (4302
10624147 T C); (4303 10624212 C T); (4304 10624229 G A); (4305 10624295 G T); (4306 10625405 G
A); (4307 10625434 C T); (4308 10625499 C T); (4309 10625625 A G); (4310 10626244 T C); (4311
10626413 A T); (4312 10626487 C G); (4313 10626670 G A); (4314 10626760 C T); (4315 10626788 G
A); (4316 10627025 A T); (4317 10627096 G A); (4318 10627380 G A); (4319 10628018 T C); (4320
10628459 T C); (4321 10628525 A C); (4322 10628882 C A); (4323 10628952 G A); (4324 10629431 A
G); (4325 10629910 G A); (4326 10630625 A G); (4327 10630994 T C); (4328 10631104 A G); (4329
10631190 G A); (4330 10632005 C T); (4331 10632316 C A); (4332 10632633 T C); (4333 10633758 C
T); (4334 10633898 C T); (4335 10634464 A C); (4336 10634869 C T); (4337 10634908 G A); (4338
10635039 AG A); (4339 10635080 A G); (4340 10635113 T C); (4341 10635460 T C); (4342 10635772 T
C); (4343 10636158 A G); (4344 10636926 C G); (4345 10636948 C T); (4346 10637054 G A); (4347
10637095 T C); (4348 10637794 A G); (4349 10637823 A AC); (4350 10637899 G A); (4351 10638053 C
T); (4352 10639076 T C); (4353 10639261 G A); (4354 10639342 G T); (4355 10640014 G A); (4356
10641296 G GT); (4357 10641492 T G); (4358 10641540 A T); (4359 10641541 A T); (4360 10641724 G
A); (4361 10641954 T G); (4362 10642694 T C); (4363 10642765 C T); (4364 10643615 C G); (4365
10643658 G A); (4366 10643681 T G); (4367 10643712 G A); (4368 10643744 G A); (4369 10644057 C
T); (4370 10644078 A G); (4371 10644741 C T); (4372 10644819 G A); (4373 10644937 G A); (4374
10645008 T C); (4375 10645019 C T); (4376 10645215 G A); (4377 10645870 A G); (4378 10645930 G
A); (4379 10646573 C T); (4380 10646666 G A); (4381 10646833 G A); (4382 10646939 T C); (4383
10647700 G A); (4384 10649409 G C); (4385 10650222 T C); (4386 10650574 T C); (4387 10651445 G
A); (4388 10651526 A G); (4389 10651990 C A); (4390 10652030 G A); (4391 10652399 C A); (4392
10652424 G A); (4393 10652850 G C); (4394 10652980 C T); (4395 10653949 G A); (4396 10653959 G
A); (4397 10653990 T C); (4398 10654083 A G); (4399 10654615 C T); (4400 10654841 T C); (4401
10654849 T C); (4402 10655167 C T); (4403 10655247 C G); (4404 10655297 T A); (4405 10655958 T
C); (4406 10656346 G A); (4407 10656901 C A); (4408 10657031 C T); (4409 10657032 A G); (4410
10657166 C T); (4411 10657508 C T); (4412 10657572 C T); (4413 10657669 C T); (4414 10657763 A
T); (4415 10657798 C T); (4416 10657810 C A); (4417 10657894 C T); (4418 10657971 G A); (4419
10658270 C A); (4420 10658327 C T); (4421 10658439 C G); (4422 10658679 G A); (4423 10658985 G
A); (4424 10659284 C T); (4425 10659501 C T); (4426 10660093 G A); (4427 10660395 T C); (4428
10660646 G A); (4429 10660765 C T); (4430 10661228 G A); (4431 10661526 C T); (4432 10661825 A
C); (4433 10661997 C T); (4434 10662422 A ACTT); (4435 10662623 A G); (4436 10662633 A G); (4437
10662702 C T); (4438 10662705 C T); (4439 10663157 G A); (4440 10663371 A G); (4441 10663582 A
G); (4442 10663900 G A); (4443 10664185 G C); (4444 10664543 A C); (4445 10664761 C A); (4446
10665075 C CT); (4447 10665264 T C); (4448 10666331 AATCG A); (4449 10666421 A G); (4450
10667066 G A); (4451 10668027 TAA T); (4452 10668521 G A); (4453 10668522 G A); (4454 10668559 T
C); (4455 10668790 G A); (4456 10668831 A G); (4457 10669378 C T); (4458 10669415 T G); (4459
10669659 T G); (4460 10669665 A G); (4461 10670488 G T); (4462 10670511 T C); (4463 10670730 G
A); (4464 10670900 T C); (4465 10670902 G A); (4466 10671206 T C); (4467 10671400 A AAT); (4468
10671548 C T); (4469 10671698 T G); (4470 10671848 GTT G); (4471 10672536 G T); (4472 10673038 A
C); (4473 10673068 C G); (4474 10673594 G A); (4475 10674057 C T); (4476 10674186 C T); (4477
10675176 G A); (4478 10675503 A C); (4479 10675636 G T); (4480 10676141 C G); (4481 10676196 T
C); (4482 10676368 A C); (4483 10676509 G A); (4484 10676561 C T); (4485 10676574 C T); (4486
10676809 C T); (4487 10676861 G A); (4488 10676897 C A); (4489 10677087 T C); (4490 10677171 C
T); (4491 10677605 C T); (4492 10678436 A AT); (4493 10678548 CA C); (4494 10679340
ATATATATATATATATTTTTTTT A); (4495 10679411 A T); (4496 10679430 G GCAC); (4497 10679591 A
G); (4498 10679659 T TA); (4499 10680842 T C); (4500 10680984 G A); (4501 10681076 C T); (4502
10681126 T C); (4503 10681519 T C); (4504 10681755 A G); (4505 10682293 T G); (4506 10682513 G
A); (4507 10682889 G C); (4508 10683075 A G); (4509 10683130 A G); (4510 10683143 G A); (4511
10683639 G A); (4512 10683880 C T); (4513 10683907 A G); (4514 10683952 T G); (4515 10683954 C
T); (4516 10683989 T C); (4517 10684031 G A); (4518 10684114 A G); (4519 10684149 A G); (4520
10684333 A G); (4521 10684414 G C); (4522 10684518 A G); (4523 10684589 A G); (4524 10684729 T
A); (4525 10684860 G A); (4526 10684959 A C); (4527 10685192 T C); (4528 10685306 G A); (4529
10685568 C T); (4530 10685648 C T); (4531 10685865 C A); (4532 10685985 T C); (4533 10686078 C
T); (4534 10686266 A G); (4535 10686427 C T); (4536 10687088 G A); (4537 10687756 C A); (4538
10688381 G A); (4539 10688506 G GA); (4540 10688733 G T); (4541 10688814 G A); (4542 10688817 G
A); (4543 10688995 C T); (4544 10689039 A G); (4545 10689412 G A); (4546 10689669 T A); (4547
10689877 G A); (4548 10690022 C T); (4549 10690105 G A); (4550 10690129 G A); (4551 10690316 G
A); (4552 10690379 C T); (4553 10690400 T C); (4554 10691006 C T); (4555 10691129 T G); (4556
10691318 G A); (4557 10691825 TA T); (4558 10691928 T A); (4559 10692752 T TA); (4560 10693866 T
A); (4561 10693867 C G); (4562 10693957 AT A); (4563 10694160 A G); (4564 10694213 A C); (4565
10694576 A T); (4566 10694845 C T); (4567 10694897 G A); (4568 10694910 G A); (4569 10695331 G
A); (4570 10695359 GA G); (4571 10695483 C T); (4572 10695517 C T); (4573 10695590 G A); (4574
10695791 CA C); (4575 10696762 C T); (4576 10696816 A G); (4577 10696882 G T); (4578 10697339 A
T); (4579 10697704 C T); (4580 10699543 G C); (4581 10700035 C T); (4582 10700166 C T); (4583
10700678 C T); (4584 10700735 C T); (4585 10701092 C A); (4586 10701309 G C); (4587 10702312 C
A); (4588 10703663 C T); (4589 10703796 C T); (4590 10704071 C T); (4591 10705465 G A); (4592
10706446 T C); (4593 10706607 A T); (4594 10706618 G A); (4595 10706917 C T); (4596 10707225 G
GA); (4597 10707238 G C); (4598 10707270 C T); (4599 10707300 G A); (4600 10707591 C T); (4601
10707798 A G); (4602 10707876 G A); (4603 10708012 TA T); (4604 10708070 A G); (4605 10708075 C
T); (4606 10708148 A G); (4607 10708348 G A); (4608 10708515 C T); (4609 10708559 C T); (4610
10709163 C T); (4611 10709587 G A); (4612 10709788 A C); (4613 10710048 G A); (4614 10710087 C
T); (4615 10710125 G A); (4616 10710138 A G); (4617 10710152 G A); (4618 10710261 T A); (4619
10710583 C T); (4620 10710603 G A); (4621 10710631 G A); (4622 10710772 G A); (4623 10710824 A
G); (4624 10710827 A C); (4625 10710896 A G); (4626 10710942 T TGGGTGATGATGATCCCG); (4627
10710992 A T); (4628 10711049 A G); (4629 10711326 G A); (4630 10711752 G A); (4631 10711753 C
G); (4632 10711834 G A); (4633 10711868 G A); (4634 10711983 T A); (4635 10711998 G A); (4636
10712145 T G); (4637 10712422 C T); (4638 10712434 T G); (4639 10712688 T C); (4640 10713076 C
T); (4641 10713512 C T); (4642 10713719 A T); (4643 10714270 T A); (4644 10714677 G A); (4645
10714742 A G); (4646 10714778 C T); (4647 10715056 C T); (4648 10715148 G T); (4649 10715165 C
T); (4650 10715204 G A); (4651 10715557 A G); (4652 10715673 C T); (4653 10715711 A G); (4654
10715748 G T); (4655 10715767 C G); (4656 10715768 C G); (4657 10716056 T C); (4658 10716097 G
A); (4659 10716141 G T); (4660 10716271 A G); (4661 10716304 A G); (4662 10716463 A G); (4663
10716490 G A); (4664 10716569 G T); (4665 10716595 A G); (4666 10716730 C T); (4667 10716758 T
C); (4668 10716760 C T); (4669 10716769 A G); (4670 10716770 C T); (4671 10716866 C T); (4672
10716954 C T); (4673 10717057 G A); (4674 10717357 A G); (4675 10717492 A G); (4676 10717538 A
G); (4677 10717565 C T); (4678 10717600 A G); (4679 10717648 G A); (4680 10717739 C A); (4681
10717973 G A); (4682 10718017 C T); (4683 10718148 G A); (4684 10718228 C T); (4685 10718284 T
C); (4686 10718319 G A); (4687 10719354 G A); (4688 10719434 C T); (4689 10719532 A G); (4690
10719602 A G); (4691 10719717 A G); (4692 10719831 G A); (4693 10719908 C T); (4694 10719984 A
G); (4695 10720348 T G); (4696 10720480 G A); (4697 10720679 G A); (4698 10720864 C T); (4699
10720972 A C); (4700 10721172 T A); (4701 10721192 T C); (4702 10721333 T C); (4703 10721632 A
G); (4704 10721919 G A); (4705 10722106 A C); (4706 10722260 A C); (4707 10722276 G T); (4708
10722277 C T); (4709 10722303 G A); (4710 10722347 C T); (4711 10722437 C T); (4712 10722766 C
T); (4713 10722792 A G); (4714 10722852 G A); (4715 10722917 A T); (4716 10722986 A G); (4717
10722994 C T); (4718 10723280 G A); (4719 10723281 G T); (4720 10723307 T C); (4721 10723346 A
G); (4722 10723350 C G); (4723 10723417 G A); (4724 10723657 A G); (4725 10723695 G C); (4726
10723937 G A); (4727 10724656 G A); (4728 10724662 C T); (4729 10724896 C A); (4730 10724946 G
A); (4731 10724975 G A); (4732 10725369 A T); (4733 10725386 G A); (4734 10725500 T C); (4735
10725786 TC T); (4736 10726088 A T); (4737 10726105 C T); (4738 10726688 C T); (4739 10726718 T
A); (4740 10726729 G A); (4741 10726738 A G); (4742 10726831 T C); (4743 10726854 G A); (4744
10726903 G A); (4745 10726930 G A); (4746 10726970 C T); (4747 10726976 C T); (4748 10726981 A
G); (4749 10727021 G A); (4750 10727070 T C); (4751 10727113 C G); (4752 10727398 C T); (4753
10727559 ATCT A); (4754 10727588 G A); (4755 10727592 T C); (4756 10728223 C A); (4757 10728347 G
T); (4758 10728495 A G); (4759 10728641 G C); (4760 10730265 A C); (4761 10730742 T C); (4762
10731045 C T); (4763 10731075 C A); (4764 10731185 C T); (4765 10731253 A G); (4766 10731319 C
T); (4767 10731762 T C); (4768 10732655 T C); (4769 10733189 A G); (4770 10733286 A T); (4771
10733779 C T); (4772 10734989 C CTAAT); (4773 10735076 T C); (4774 10735094 T A); (4775 10735142
C T); (4776 10735308 A G); (4777 10735479 T C); (4778 10735513 G A); (4779 10735574 G A); (4780
10735580 T C); (4781 10735772 A G); (4782 10735774 T C); (4783 10735775 G T); (4784 10735895 T
C); (4785 10736077 C G); (4786 10736448 A C); (4787 10737096 T C); (4788 10737579 G C); (4789
10738172 C T); (4790 10738195 C T); (4791 10738508 C T); (4792 10738712 A G); (4793 10738780 C
T); (4794 10738810 C T); (4795 10739001 A C); (4796 10739072 G A); (4797 10739120 T C); (4798
10740057 T C); (4799 10740144 C T); (4800 10740508 C G); (4801 10740708 G A); (4802 10740742 C
A); (4803 10740997 T C); (4804 10741019 C T); (4805 10741047 A C); (4806 10741062 G C); (4807
10741106 A G); (4808 10741177 C T); (4809 10741258 G A); (4810 10741409 A C); (4811 10741512 T
C); (4812 10741744 A G); (4813 10741806 T C); (4814 10742026 C T); (4815 10742040 G A); (4816
10742148 G A); (4817 10742577 C T); (4818 10742799 A G); (4819 10742935 A G); (4820 10742980 G
C); (4821 10743117 C T); (4822 10743146 T A); (4823 10743183 G A); (4824 10743190 T C); (4825
10743205 C T); (4826 10743364 C G); (4827 10743871 C T); (4828 10743978 A G); (4829 10744069 G
A); (4830 10744310 G A); (4831 10744959 T C); (4832 10745059 C A); (4833 10745436 A AC); (4834
10745503 C T); (4835 10745654 G C); (4836 10745746 C T); (4837 10746173 G A); (4838 10746261 A
C); (4839 10746415 C T); (4840 10746558 G A); (4841 10746814 C T); (4842 10746974 T C); (4843
10747143 C T); (4844 10747198 T C); (4845 10747422 A C); (4846 10747505 G A); (4847 10747523 T
C); (4848 10747724 C A); (4849 10747930 T C); (4850 10747999 G A); (4851 10748099 G A); (4852
10748194 A G); (4853 10748204 C G); (4854 10748250 T C); (4855 10748374 G A); (4856 10748407 G
A); (4857 10748497 C T); (4858 10748839 C T); (4859 10749126 A G); (4860 10749214 TG T); (4861
10749274 G A); (4862 10749539 C T); (4863 10749743 C G); (4864 10749817 C A); (4865 10749874 C
T); (4866 10750088 T C); (4867 10750296 C T); (4868 10750325 C T); (4869 10750540 C T); (4870
10750678 T C); (4871 10750718 T A); (4872 10750849 G C); (4873 10750856 T G); (4874 10750857 C
A); (4875 10750860 C A); (4876 10750868 G A); (4877 10750906 C T); (4878 10751079 T G); (4879
10751099 G A); (4880 10751154 G A); (4881 10751172 A C); (4882 10751188 G A); (4883 10751202 C
T); (4884 10751211 A C); (4885 10751325 C T); (4886 10751328 A G); (4887 10751337 C G); (4888
10751448 C T); (4889 10751556 A C); (4890 10751661 G A); (4891 10752545 C G); (4892 10752827 C
T); (4893 10753178 C T); (4894 10753196 G A); (4895 10753231 C T); (4896 10753269 A G); (4897
10753291 T TG); (4898 10753588 A C); (4899 10753949 A G); (4900 10755910 G A); (4901 10757494 C
T); (4902 10757664 A G); (4903 10758482 G T); (4904 10758510 A G); (4905 10758823 C T); (4906
10758848 C T); (4907 10758857 G C); (4908 10758885 A C); (4909 10760046 A G); (4910 10760229 A
C); (4911 10760268 C T); (4912 10760276 T C); (4913 10760297 A G); (4914 10760593 G A); (4915
10760614 A G); (4916 10760705 C T); (4917 10760726 C T); (4918 10760905 A G); (4919 10761349 A
G); (4920 10761671 T C); (4921 10761708 T C); (4922 10761772 A G); (4923 10761864 T C); (4924
10761865 G T); (4925 10761869 C G); (4926 10761895 TTGTCTTTGTTGTATCTGAGTTG T); (4927
10761902 T C); (4928 10761995 C T); (4929 10762001 C T); (4930 10762087 A G); (4931 10762093 A
G); (4932 10762113 C T); (4933 10762157 G A); (4934 10762224 C T); (4935 10762462 C T); (4936
10762572 T G); (4937 10762933 A G); (4938 10763067 G A); (4939 10763128 C A); (4940 10763145 T
C); (4941 10763165 C A); (4942 10763362 C T); (4943 10763875 G A); (4944 10763950 G A); (4945
10764095 G T); (4946 10764104 G T); (4947 10764344 C CT); (4948 10764728 G A); (4949 10764898 C
T); (4950 10765034 G A); (4951 10765408 T A); (4952 10765540 G A); (4953 10766489 G T); (4954
10766499 G T); (4955 10767550 C A); (4956 10767805 T A); (4957 10768139 G A); (4958 10769189 G
T); (4959 10769828 A T); (4960 10769832 TA T); (4961 10769845 T C); (4962 10769850 A T); (4963
10769986 A AGTC); (4964 10770037 A AT); (4965 10770048 A G); (4966 10770240 A C); (4967 10770375
C T); (4968 10770607 C T); (4969 10770652 G A); (4970 10770751 G GT); (4971 10770778 T C); (4972
10770802 C G); (4973 10770941 C T); (4974 10771086 T A); (4975 10771167 A G); (4976 10771258 G
A); (4977 10771693 G A); (4978 10771905 C T); (4979 10771992 C T); (4980 10772066 G A); (4981
10772398 T C); (4982 10774019 C T); (4983 10774658 A G); (4984 10774720 T G); (4985 10774737 A
G); (4986 10775080 TTC T); (4987 10775639 A T); (4988 10775872 C A); (4989 10776492 A G); (4990
10776652 G A); (4991 10776674 A ATT); (4992 10776760 G A); (4993 10777065 C T); (4994 10778779 C
A); (4995 10778951 C T); (4996 10779397 C T); (4997 10779591 C T); (4998 10779924 G A); (4999
10781323 A T); (5000 10781352 TA T); (5001 10781403 A G); (5002 10781569 T G); (5003 10781978 C
T); (5004 10782461 G A); (5005 10782654 G T); (5006 10782656 A C); (5007 10782691 T C); (5008
10782862 G GA); (5009 10783878 G A); (5010 10784260 C CTTTTCTTTTATCTTATTTT); (5011 10784973 G
T); (5012 10785003 C T); (5013 10785008 A G); (5014 10785097 C T); (5015 10785121 T C); (5016
10785517 A G); (5017 10786371 C T); (5018 10786586 G A); (5019 10787179 T C); (5020 10787341 A
T); (5021 10787352 C T); (5022 10787442 G A); (5023 10787562 G A); (5024 10787878 G A); (5025
10788151 G A); (5026 10788318 G T); (5027 10789583 G A); (5028 10789892 G A); (5029 10789947 C
T); (5030 10790147 A T); (5031 10790401 A G); (5032 10790910 C T); (5033 10791059 A G); (5034
10791213 G A); (5035 10791310 T C); (5036 10791380 T A); (5037 10791401 G A); (5038 10791494 C
T); (5039 10791507 C T); (5040 10791555 T C); (5041 10791581 G A); (5042 10791767 C T); (5043
10793312 C A); (5044 10793343 C G); (5045 10793427 A G); (5046 10793446 A G); (5047 10793476 A
C); (5048 10793479 T G); (5049 10793486 G T); (5050 10793487 T A); (5051 10793497 G A); (5052
10793499 A G); (5053 10793507 A G); (5054 10793596 C A); (5055 10793797 A T); (5056 10793805 G
T); (5057 10793823 A G); (5058 10793828 G A); (5059 10793829 T G); (5060 10796648 TG T); (5061
10798090 G A); (5062 10798512 A G); (5063 10799081 T C); (5064 10804074 T G); (5065 10804264 C
T); (5066 10804366 GT G); (5067 10804367 T C); (5068 10805745 G C); (5069 10805944 A C); (5070
10805963 T A); (5071 10806224 C T); (5072 10806294 G A); (5073 10807195 A T); (5074 10807197 G
GACTTGGTCCATGCCGAAT); (5075 10807380 A G); (5076 10807467 A G); (5077 10808141 C G); (5078
10808243 G T); (5079 10808562 C T); (5080 10808756 T C); (5081 10809134 G A); (5082 10809630 C
T); (5083 10809634 A G); (5084 10809781 A G); (5085 10809837 T C); (5086 10809939 G A); (5087
10810147 T C); (5088 10811973 G T); (5089 10812074 T C); (5090 10812201 C T); (5091 10812309 G
A); (5092 10812350 C T); (5093 10812449 G A); (5094 10812477 A G); (5095 10812907 A G); (5096
10813051 T C); (5097 10813303 C T); (5098 10813592 C T); (5099 10813850 C T); (5100 10813985 T
C); (5101 10814222 A G); (5102 10815126 G A); (5103 10815190 C T); (5104 10815242 T TCTTTTC); (5105
10815598 G A); (5106 10815620 C T); (5107 10816252 C G); (5108 10817292 G C); (5109 10817592 G
A); (5110 10817624 G A); (5111 10817703 T C); (5112 10817883 T C); (5113 10818106 C T); (5114
10818549 G T); (5115 10818591 C A); (5116 10819847 C A); (5117 10819853 G A); (5118 10820187 C
T); (5119 10820375 AT A); (5120 10822204 C T); (5121 10822896 G A); (5122 10823141 C T); (5123
10823256 C T); (5124 10823279 G A); (5125 10823605 C T); (5126 10823640 G A); (5127 10823737 A
G); (5128 10823913 T A); (5129 10823931 C T); (5130 10824574 G GT); (5131 10824717 C T); (5132
10824823 C T); (5133 10825068 G A); (5134 10825244 T C); (5135 10825247 G A); (5136 10826331 G
A); (5137 10826593 C T); (5138 10826600 G A); (5139 10826715 C T); (5140 10828353 G A); (5141
10828414 C T); (5142 10828508 G A); (5143 10829215 A G); (5144 10830347 C A); (5145 10831333 C
T); (5146 10831409 A G); (5147 10831433 C T); (5148 10831533 C T); (5149 10832017 C T); (5150
10832133 C T); (5151 10832260 C T); (5152 10832300 G A); (5153 10832550 T C); (5154 10832584 G
A); (5155 10833393 T C); (5156 10833575 G A); (5157 10833609 A G); (5158 10834108 C T); (5159
10834339 C A); (5160 10834376 T C); (5161 10834589 C CT); (5162 10834770 G A); (5163 10835184 C
T); (5164 10835276 C A); (5165 10835888 G A); (5166 10837791 T TA); (5167 10838232 A G); (5168
10840073 CTTA C); (5169 10840542 C T); (5170 10841146 T C); (5171 10841244 G A); (5172 10841287 C
T); (5173 10841351 C T); (5174 10841521 A G); (5175 10841552 A G); (5176 10841832 C T); (5177
10842004 G A); (5178 10842294 A G); (5179 10843136 G A); (5180 10844045 T G); (5181 10844929 G
A); (5182 10845102 T C); (5183 10845880 C T); (5184 10846061 T G); (5185 10846449 C T); (5186
10847991 A T); (5187 10848664 T G); (5188 10849715 C G); (5189 10850945 T C); (5190 10850956 A
G); (5191 10851072 A T); (5192 10851113 A T); (5193 10851161 C T); (5194 10851710 G A); (5195
10851725 G A); (5196 10851986 G A); (5197 10851987 C T); (5198 10852013 C T); (5199 10852201 A
T); (5200 10852345 G A); (5201 10852745 T C); (5202 10853085 A AT); (5203 10853364 C T); (5204
10854404 G A); (5205 10855281 TA T); (5206 10855466 T A); (5207 10855549 G A); (5208 10855609 G
T); (5209 10855610 A T); (5210 10855767 TACTAAGTAAAACA T); (5211 10855813 A C); (5212 10857622
C T); (5213 10857663 C A); (5214 10857911 T TA); (5215 10857990 C A); (5216 10858044 G A); (5217
10858096 CA C); (5218 10858115 G A); (5219 10858146 C T); (5220 10858455 G C); (5221 10858657 T
G); (5222 10858732 C G); (5223 10858960 G A); (5224 10858971 C T); (5225 10858994 T C); (5226
10858998 G C); (5227 10859013 A G); (5228 10859015 T A); (5229 10859047 C T); (5230 10859067 C
T); (5231 10859117 C T); (5232 10859304 A G); (5233 10859330 C T); (5234 10859573 G T); (5235
10859687 T A); (5236 10859744 T TA); (5237 10859755 G A); (5238 10859870 A T); (5239 10860117 A
G); (5240 10860118 G T); (5241 10860132 T C); (5242 10860174 G A); (5243 10860253 G A); (5244
10860318 G A); (5245 10861973 A G); (5246 10862100 C T); (5247 10864436 C
CTACCTCACTTCACA); (5248 10864540 C A); (5249 10864643 G C); (5250 10864648 C T); (5251 10864926
C T); (5252 10864980 A G); (5253 10865113 A T); (5254 10865224 T C); (5255 10865229 A T); (5256
10865787 CAA C); (5257 10865949 G A); (5258 10865950 T G); (5259 10865974 T G); (5260 10866142 C
T); (5261 10866171 G A); (5262 10866239 A G); (5263 10866240 C T); (5264 10866246 G T); (5265
10866262 C A); (5266 10866349 T C); (5267 10866403 G A); (5268 10866419 C T); (5269 10866483 G
C); (5270 10866493 A G); (5271 10866856 C T); (5272 10867054 C T); (5273 10867072 C G); (5274
10867158 G A); (5275 10867232 T G); (5276 10867286 G A); (5277 10867294 C T); (5278 10867366 A
G); (5279 10867536 G A); (5280 10867767 C T); (5281 10867843 T C); (5282 10867920 T C); (5283
10867950 C T); (5284 10868018 A G); (5285 10868149 T C); (5286 10868154 G T); (5287 10868159 G
T); (5288 10868268 G A); (5289 10868360 A G); (5290 10868494 A G); (5291 10868535 C T); (5292
10868596 C A); (5293 10868663 C T); (5294 10869105 G A); (5295 10869130 G C); (5296 10869162 C
A); (5297 10869254 G A); (5298 10869377 G A); (5299 10869519 AG A); (5300 10869763 A G); (5301
10869789 C T); (5302 10869844 C T); (5303 10870180 A C); (5304 10871113 G A); (5305 10871240 T
C); (5306 10872895 C T); (5307 10872960 C T); (5308 10873039 C T); (5309 10873081 A G); (5310
10873095 C A); (5311 10873096 T C); (5312 10873343 T A); (5313 10873458 C T); (5314 10873473 A
AT); (5315 10873526 A T); (5316 10873640 C A); (5317 10874212 T C); (5318 10874227 C G); (5319
10874231 A G); (5320 10874265 C T); (5321 10874721 A G); (5322 10874770 C G); (5323 10874789 C
T); (5324 10874964 A G); (5325 10874998 A AT); (5326 10875027 C T); (5327 10875079 G A); (5328
10875110 C T); (5329 10875131 AT A); (5330 10875143 C CA); (5331 10875181 C T); (5332 10875235 G
T); (5333 10875321 A AT); (5334 10875562 G T); (5335 10875603 G A); (5336 10877412 T G); (5337
10877475 AAGTTGTTTTACTT A); (5338 10877918 T C); (5339 10877930 G A); (5340 10877948 G
A); (5341 10878077 A G); (5342 10878106 C T); (5343 10878185 T C); (5344 10878213 T C); (5345
10878287 A G); (5346 10878438 T A); (5347 10878862 T G); (5348 10879412 T A); (5349 10879649 C
A); (5350 10879720 A G); (5351 10879760 G C); (5352 10879908 TTATA T); (5353 10879979 A T); (5354
10880001 A T); (5355 10880169 TA T); (5356 10880254 C T); (5357 10880260 T
TGACATGACATTGTAGG); (5358 10880271 A T); (5359 10880313 G A); (5360 10880321 T G); (5361
10880324 T G); (5362 10880369 C T); (5363 10880384 T G); (5364 10880408 T A); (5365 10880415 C
CTG); (5366 10880425 T A); (5367 10880427 A G); (5368 10880470 A G); (5369 10880521 G T); (5370
10880528 T A); (5371 10880576 G T); (5372 10880684 A G); (5373 10880948 T G); (5374 10881334 G
C); (5375 10881474 A G); (5376 10881586 T C); (5377 10881749 C A); (5378 10881948 A C); (5379
10882677 A G); (5380 10882680 A G); (5381 10882689 T C); (5382 10882748 A G); (5383 10882896 C
T); (5384 10883000 G A); (5385 10883201 A G); (5386 10883324 T G); (5387 10883383 C T); (5388
10883517 G A); (5389 10883522 A G); (5390 10883705 G A); (5391 10883799 G A); (5392 10884172 T
G); (5393 10884726 A G); (5394 10884777 C T); (5395 10884863 T C); (5396 10884981 A T); (5397
10885127 A G); (5398 10885728 C T); (5399 10885790 T C); (5400 10886248 A C); (5401 10886413 G
A); (5402 10886487 G A); (5403 10886492 C T); (5404 10886511 A G); (5405 10886596 T A); (5406
10886940 T C); (5407 10886943 T G); (5408 10887031 A T); (5409 10887143 G A); (5410 10887273 C
T); (5411 10887310 T A); (5412 10887714 G A); (5413 10887793 A T); (5414 10888087 G A); (5415
10888214 G C); (5416 10888410 A G); (5417 10888600 GTTAA G); (5418 10888926 A G); (5419 10888969
AC A); (5420 10888977 C T); (5421 10888992 G T); (5422 10889065 G A); (5423 10889084 T A); (5424
10889113 G A); (5425 10889159 C T); (5426 10889446 G A); (5427 10889546 T C); (5428 10889678 G
A); (5429 10889822 T C); (5430 10890713 T C); (5431 10890738 T C); (5432 10890748 A G); (5433
10890772 C T); (5434 10890849 G A); (5435 10890850 T C); (5436 10891235 T TCAC); (5437 10891278 G
A); (5438 10891290 G A); (5439 10891302 G T); (5440 10891439 G A); (5441 10891598 A G); (5442
10891612 T C); (5443 10891971 A G); (5444 10892161 A G); (5445 10892215 C A); (5446 10892326 G
A); (5447 10892592 C A); (5448 10892612 C T); (5449 10892621 T C); (5450 10892735 A G); (5451
10892762 G T); (5452 10892843 C T); (5453 10892926 C T); (5454 10892960 T C); (5455 10892975 A
G); (5456 10892980 T C); (5457 10892981 A G); (5458 10893019 A G); (5459 10893511 G A); (5460
10893534 C T); (5461 10893575 C G); (5462 10893674 T C); (5463 10893729 A G); (5464 10893805 T
C); (5465 10893843 C T); (5466 10893953 A G); (5467 10894331 C T); (5468 10894360 C T); (5469
10894397 A G); (5470 10894447 A T); (5471 10894471 G A); (5472 10894706 A G); (5473 10894735 C
T); (5474 10895565 G C); (5475 10896318 T C); (5476 10896342 T A); (5477 10896393 A G); (5478
10896505 G T); (5479 10896525 C T); (5480 10896660 C A); (5481 10896782 G A); (5482 10896885 A
G); (5483 10896953 T C); (5484 10897118 G A); (5485 10897141 C T); (5486 10897273 C T); (5487
10897308 T C); (5488 10897360 C T); (5489 10897366 T G); (5490 10897413 C T); (5491 10897819 T
G); (5492 10897920 C T); (5493 10897940 C A); (5494 10897970 A T); (5495 10898131 T C); (5496
10898292 A C); (5497 10898310 G A); (5498 10898313 T C); (5499 10898315 A G); (5500 10898494 A
G); (5501 10898970 C T); (5502 10899045 T C); (5503 10899052 T G); (5504 10899054 G A); (5505
10899057 TA T); (5506 10899144 T C); (5507 10899212 G A); (5508 10899257 T C); (5509 10899300 T
C); (5510 10899626 G A); (5511 10899731 G C); (5512 10899818 G C); (5513 10899830 G A); (5514
10899854 T C); (5515 10899907 G GC); (5516 10900401 T A); (5517 10900632 A C); (5518 10900689 A
G); (5519 10900757 A T); (5520 10900818 A G); (5521 10900835 A G); (5522 10900894 A T); (5523
10900929 A G); (5524 10901117 G A); (5525 10901191 T C); (5526 10901306 T C); (5527 10901307 G
A); (5528 10901318 T A); (5529 10901330 A G); (5530 10901364 G A); (5531 10901384 T G); (5532
10901416 G T); (5533 10901551 G A); (5534 10901605 T C); (5535 10901620 T C); (5536 10901649 G
A); (5537 10901910 A G); (5538 10901913 T C); (5539 10901923 G A); (5540 10901952 A T); (5541
10902151 T C); (5542 10902249 C G); (5543 10902368 A G); (5544 10902387 C T); (5545 10902559 T
C); (5546 10902613 G T); (5547 10902678 T G); (5548 10902795 A G); (5549 10902899 T C); (5550
10902904 G A); (5551 10902929 AC A); (5552 10902957 G A); (5553 10902980 C T); (5554 10903066 C
T); (5555 10903103 T C); (5556 10903112 T C); (5557 10903174 G A); (5558 10903185 C T); (5559
10903236 C A); (5560 10903286 C T); (5561 10903321 G A); (5562 10903885 A G); (5563 10903900 T
C); (5564 10903996 G C); (5565 10904010 T C); (5566 10904043 T A); (5567 10904080 C T); (5568
10904595 G C); (5569 10905122 G T); (5570 10905156 A G); (5571 10905187 ATC A); (5572 10905324
GA G); (5573 10905540 T C); (5574 10905840 C T); (5575 10906084 T TC); (5576 10906401 G C); (5577
10906552 T C); (5578 10906674 T C); (5579 10906721 AACT A); (5580 10907123 G T); (5581 10907161 C
T); (5582 10907207 A C); (5583 10907220 G A); (5584 10907270 A AT); (5585 10907493 A G); (5586
10907544 C T); (5587 10907547 G A); (5588 10907595 T C); (5589 10907665 T A); (5590 10907666 T
A); (5591 10907684 T C); (5592 10907795 C T); (5593 10907830 T C); (5594 10907844 G A); (5595
10907888 T C); (5596 10907913 A G); (5597 10907918 A G); (5598 10907978 A C); (5599 10908073 C
A); (5600 10908106 C T); (5601 10908254 T C); (5602 10908417 T A); (5603 10909125 A G); (5604
10909216 G T); (5605 10909270 C T); (5606 10909272 T C); (5607 10909303 A C); (5608 10909794 A
G); (5609 10909819 G A); (5610 10909821 T C); (5611 10909843 A G); (5612 10909863 C T); (5613
10909899 C T); (5614 10910214 T C); (5615 10910352 A T); (5616 10910362 A G); (5617 10910434 T
A); (5618 10911047 G A); (5619 10911230 A C); (5620 10911459 G A); (5621 10911637 G C); (5622
10911701 C T); (5623 10911740 C T); (5624 10911800 A C); (5625 10911950 A C); (5626 10912005 T
C); (5627 10912169 T C); (5628 10912222 G A); (5629 10912268 A G); (5630 10912546 C
CGATGAATTATTTTCCATTGTCT); (5631 10912548 C G); (5632 10912549 C A); (5633 10912597 T C); (5634
10912601 A G); (5635 10912604 G A); (5636 10912606 A G); (5637 10912616 C T); (5638 10912634 G
A); (5639 10912676 G A); (5640 10912696 T C); (5641 10912761 T C); (5642 10912852 A G); (5643
10912999 C T); (5644 10913137 C T); (5645 10913212 C T); (5646 10913332 G T); (5647 10913337 G
T); (5648 10913416 T C); (5649 10913475 T G); (5650 10913477 T C); (5651 10913528 C T); (5652
10913653 A T); (5653 10913666 A G); (5654 10914119 G A); (5655 10914529 T C); (5656 10914545 T
G); (5657 10914553 T C); (5658 10915247 T G); (5659 10915269 G A); (5660 10915348 G A); (5661
10915375 T C); (5662 10915416 G T); (5663 10915461 G A); (5664 10915513 A G); (5665 10915555 A
G); (5666 10915585 A G); (5667 10915624 C T); (5668 10915637 A G); (5669 10915672 G C); (5670
10915678 G C); (5671 10915705 C T); (5672 10915846 T A); (5673 10915859 A G); (5674 10915902 T
G); (5675 10916088 G A); (5676 10916166 T C); (5677 10916224 G A); (5678 10916276 C T); (5679
10916300 C T); (5680 10916320 C T); (5681 10916326 G A); (5682 10916338 G T); (5683 10916401 G
A); (5684 10916442 C T); (5685 10916501 G A); (5686 10916502 A C); (5687 10916518 G A); (5688
10916521 A G); (5689 10916549 T C); (5690 10916595 C G); (5691 10916602 T C); (5692 10916617 A
G); (5693 10916626 T C); (5694 10916660 G T); (5695 10916662 T C); (5696 10916765 C T); (5697
10916775 G A); (5698 10916813 C T); (5699 10916850 G T); (5700 10916852 A C); (5701 10916866 T
C); (5702 10917034 T C); (5703 10917071 C G); (5704 10917078 G A); (5705 10917122 A G); (5706
10917141 G A); (5707 10917163 C G); (5708 10917195 A G); (5709 10917208 T A); (5710 10917285 G
A); (5711 10917290 A G); (5712 10917292 C T); (5713 10917388 T A); (5714 10917405 G A); (5715
10917523 A G); (5716 10917531 C T); (5717 10917542 T C); (5718 10917569 T C); (5719 10917590 A
G); (5720 10917620 A G); (5721 10917661 A G); (5722 10917677 T C); (5723 10917697 A G); (5724
10917708 T C); (5725 10917721 A G); (5726 10917732 T G); (5727 10917733 G C); (5728 10917744 T
C); (5729 10917765 T C); (5730 10917853 G A); (5731 10917884 T C); (5732 10917915 T G); (5733
10917962 A G); (5734 10918309 G A); (5735 10918448 G T); (5736 10918559 G A); (5737 10918732 T
A); (5738 10918748 A T); (5739 10918783 G A); (5740 10918820 T C); (5741 10919233 A C); (5742
10919480 A C); (5743 10919552 A G); (5744 10920209 TA T); (5745 10920379 C A); (5746 10920492 C
A); (5747 10921864 A T); (5748 10922082 A G); (5749 10923110 T A); (5750 10923113 A T); (5751
10923465 C CAG); (5752 10924016 G C); (5753 10924441 T TA); (5754 10924947 T C); (5755 10925658 G
A); (5756 10925801 C T); (5757 10926264 C T); (5758 10926550 C T); (5759 10926551 A G); (5760
10926571 A T); (5761 10928276 G A); (5762 10928638 T C); (5763 10928839 G A); (5764 10929962 T
G); (5765 10930200 G A); (5766 10930639 G A); (5767 10930769 A G); (5768 10930976 T C); (5769
10931246 AT A); (5770 10931338 G A); (5771 10931347 C T); (5772 10931423 T C); (5773 10931778 A
G); (5774 10932060 C T); (5775 10932128 T A); (5776 10932204 CA C); (5777 10932553 T G); (5778
10933030 C T); (5779 10933110 C T); (5780 10933253 G A); (5781 10933390 G C); (5782 10933445 T
C); (5783 10933479 G A); (5784 10933810 A G); (5785 10934159 A G); (5786 10934300 T G); (5787
10934380 A G); (5788 10934420 C CG); (5789 10934485 G A); (5790 10935252 A C); (5791 10935310 C
T); (5792 10935949 T G); (5793 10935958 A G); (5794 10936169 G T); (5795 10936616 G A); (5796
10937639 T G); (5797 10938028 C T); (5798 10938045 G A); (5799 10938471 G A); (5800 10938574 A
G); (5801 10939492 T G); (5802 10939828 G A); (5803 10939839 C G); (5804 10939979 G A); (5805
10940016 C T); (5806 10940020 G A); (5807 10940192 C T); (5808 10940205 A G); (5809 10940381 C
T); (5810 10940561 C T); (5811 10940628 T G); (5812 10940800 A G); (5813 10941155 T C); (5814
10941560 G A); (5815 10941597 C T); (5816 10941629 C T); (5817 10942470 C T); (5818 10942886 G
A); (5819 10943050 C T); (5820 10943076 G T); (5821 10943170 C T); (5822 10943423 T C); (5823
10943506 G GTA); (5824 10943546 C G); (5825 10943667 G A); (5826 10944441 C A); (5827 10945102 T
C); (5828 10945732 C T); (5829 10946097 C T); (5830 10946459 C T); (5831 10946700 A G); (5832
10946818 A T); (5833 10947714 C CT); (5834 10947789 C T); (5835 10948117 CA C); (5836 10948931 C
A); (5837 10948974 C T); (5838 10949267 A C); (5839 10949434 G T); (5840 10949594 C T); (5841
10949657 G T); (5842 10950073 A G); (5843 10950083 G A); (5844 10950168 T C); (5845 10951752 T
G); (5846 10951974 G A); (5847 10952005 G A); (5848 10952390 G T); (5849 10952697 A T); (5850
10954623 G A); (5851 10956316 G C); (5852 10957399 C T); (5853 10958677 CT C); (5854 10958876 C
CT); (5855 10959154 T C); (5856 10959743 A G); (5857 10960104 CAG C); (5858 10960770 C T); (5859
10960997 A AAC); (5860 10961033 C T); (5861 10961133 C G); (5862 10961138 T C); (5863 10961155 A
G); (5864 10961176 T C); (5865 10961238 C T); (5866 10961688 T A); (5867 10961942 C T); (5868
10962279 C T); (5869 10962449 G A); (5870 10962477 G A); (5871 10962520 T C); (5872 10962603 G
A); (5873 10962623 C T); (5874 10962697 T A); (5875 10963127 T C); (5876 10963173 G C); (5877
10963350 A G); (5878 10963597 G C); (5879 10963852 G A); (5880 10964006 C CTATT); (5881 10964727
A T); (5882 10964744 G A); (5883 10965873 T G); (5884 10966748 G A); (5885 10967208 G T); (5886
10967260 T C); (5887 10967585 C T); (5888 10967724 G A); (5889 10968866 A AT); (5890 10969061 T
A); (5891 10970097 G A); (5892 10971348 T C); (5893 10971364 G T); (5894 10971380 T G); (5895
10971436 C A); (5896 10971548 T TA); (5897 10972163 A AT); (5898 10972280 A T); (5899 10972356 C
G); (5900 10972779 T TATG); (5901 10973623 G A); (5902 10973730 G A); (5903 10973756 T C); (5904
10973767 T C); (5905 10973772 G A); (5906 10973800 C G); (5907 10973871 A G); (5908 10973944 C
T); (5909 10973956 A C); (5910 10974007 T C); (5911 10974020 C A); (5912 10974081 T C); (5913
10974088 G C); (5914 10974252 C T); (5915 10974288 A AGT); (5916 10974397 C T); (5917 10974426 G
A); (5918 10974463 G A); (5919 10974498 A G); (5920 10974560 C T); (5921 10974639 A G); (5922
10974815 G A); (5923 10975225 A T); (5924 10975444 T C); (5925 10975610 AT A); (5926 10975818 A
AT); (5927 10976802 A T); (5928 10976819 A AGC); (5929 10976918 T C); (5930 10977017 T C); (5931
10977248 C T); (5932 10977302 T C); (5933 10977303 C T); (5934 10977319 G A); (5935 10977424 T
A); (5936 10977524 A T); (5937 10977557 A G); (5938 10977583 T TAA); (5939 10977694 C T); (5940
10977840 A G); (5941 10977856 T C); (5942 10977946 T C); (5943 10978070 GT G); (5944 10978310 A
G); (5945 10978493 G A); (5946 10978504 G A); (5947 10978674 G A); (5948 10978691 G A); (5949
10978699 T C); (5950 10978791 T C); (5951 10978972 T C); (5952 10979302 T A); (5953 10979667 G
A); (5954 10979814 A AG); (5955 10980015 T A); (5956 10980193 T C); (5957 10980369 A T); (5958
10980384 T C); (5959 10980462 G T); (5960 10980583 G A); (5961 10980673 A T); (5962 10980911 G
A); (5963 10981080 T C); (5964 10981112 A G); (5965 10981118 G C); (5966 10981241 C A); (5967
10981484 A T); (5968 10982123 A C); (5969 10982259 G C); (5970 10982402 A T); (5971 10982412 A
G); (5972 10982417 G A); (5973 10982519 T G); (5974 10982555 C T); (5975 10982588 G C); (5976
10982669 C T); (5977 10982675 C T); (5978 10982706 A G); (5979 10982832 G A); (5980 10983931 A
G); (5981 10984107 A G); (5982 10984214 T A); (5983 10984224 A C); (5984 10985123 A C); (5985
10985179 G A); (5986 10985191 G A); (5987 10985201 T C); (5988 10985310 A G); (5989 10985341 T
C); (5990 10985612 T C); (5991 10985891 A G); (5992 10985901 C T); (5993 10985914 G C); (5994
10985990 A C); (5995 10987015 C T); (5996 10987202 T C); (5997 10987380 T G); (5998 10987397 G
A); (5999 10987446 C T); (6000 10987490 C T); (6001 10987564 TA T); (6002 10987797 A G); (6003
10989394 T G); (6004 10989528 G A); (6005 10989543 C T); (6006 10990051 G C); (6007 10991111 C
T); (6008 10991141 G C); (6009 10991157 A G); (6010 10991508 G A); (6011 10991652 G A); (6012
10992023 T C); (6013 10992166 C T); (6014 10992290 T C); (6015 10992333 A G); (6016 10992861 A
G); (6017 10992908 C A); (6018 10993386 C T); (6019 10993532 G A); (6020 10993535 G T); (6021
10993574 C T); (6022 10993629 G T); (6023 10993630 A T); (6024 10993684 G A); (6025 10993767 C
A); (6026 10993823 G T); (6027 10994274 C A); (6028 10994330 A C); (6029 10994405 C T); (6030
10994442 G C); (6031 10994540 A G); (6032 10994573 G A); (6033 10994792 T G); (6034 10995483 C
T); (6035 10995596 C G); (6036 10995607 G A); (6037 10995621 T C); (6038 10996046 T G); (6039
10996086 G T); (6040 10996196 T C); (6041 10996229 G A); (6042 10997183 A C); (6043 10997224 A
G); (6044 10997239 A G); (6045 10997272 GA G); (6046 10997454 T G); (6047 10997747 T C); (6048
10997968 G T); (6049 10997997 A G); (6050 10998057 T C); (6051 10998222 A G); (6052 10998404
GTGA G); (6053 10998438 A G); (6054 10999189 T G); (6055 10999199 A G); (6056 10999218 T C); (6057
10999299 T C); (6058 10999348 G A); (6059 10999501 G A); (6060 11000193 C T); (6061 11000312 C
T); (6062 11000338 G A); (6063 11000651 A G); (6064 11001127 C T); (6065 11001573 A T); (6066
11001641 T C); (6067 11001786 A T); (6068 11001949 T C); (6069 11002106 A G); (6070 11002492 A
G); (6071 11002586 A G); (6072 11002746 T TAAG); (6073 11003231 A T); (6074 11003611 T G); (6075
11003859 G C); (6076 11003886 GC G); (6077 11003991 A T); (6078 11004326 A G); (6079 11004341 G
A); (6080 11004513 G A); (6081 11005349 C T); (6082 11005753 T C); (6083 11006103 T C); (6084
11006142 A C); (6085 11006143 T A); (6086 11006149 T C); (6087 11006336 A T); (6088 11006417 T
G); (6089 11006454 C A); (6090 11006458 T A); (6091 11006539 C T); (6092 11006785 A G); (6093
11006811 C T); (6094 11006839 G A); (6095 11007246 A G); (6096 11007306 G A); (6097 11010436 C
T); (6098 11010641 C G); (6099 11010841 C T); (6100 11011054 T C); (6101 11011685 G A); (6102
11011746 G A); (6103 11012206 G A); (6104 11012340 A C); (6105 11012463 C T); (6106 11012508 GT
G); (6107 11012709 C G); (6108 11012947 A G); (6109 11013141 A G); (6110 11013173 G A); (6111
11013222 G A); (6112 11013391 G A); (6113 11013628 C T); (6114 11013849 A G); (6115 11014227 A
C); (6116 11014298 T TA); (6117 11014374 G A); (6118 11014517 C T); (6119 11014763 G A); (6120
11015388 T C); (6121 11016087 G A); (6122 11016978 C T); (6123 11017058 A G); (6124 11017106 C
T); (6125 11017572 A G); (6126 11017637 T C); (6127 11017882 A G); (6128 11017971 C A); (6129
11018108 G A); (6130 11018446 C A); (6131 11018735 T G); (6132 11018840 A G); (6133 11019254 G
A); (6134 11019351 T C); (6135 11019453 G A); (6136 11019457 C T); (6137 11019512 G A); (6138
11019699 C T); (6139 11019718 C G); (6140 11019803 C T); (6141 11019916 C T); (6142 11020165 T
G); (6143 11020179 C T); (6144 11020276 C T); (6145 11020376 G C); (6146 11020407 C T); (6147
11020836 A G); (6148 11021043 A G); (6149 11021265 G A); (6150 11021278 A G); (6151 11021336 A
C); (6152 11021429 G T); (6153 11021449 T G); (6154 11022093 A G); (6155 11022514 G A); (6156
11022517 A C); (6157 11023094 G A); (6158 11023227 A C); (6159 11023406 C T); (6160 11023584 T
G); (6161 11023669 C G); (6162 11023746 AC A); (6163 11023771 T C); (6164 11023964 A C); (6165
11024140 C T); (6166 11024300 G A); (6167 11024308 T A); (6168 11024336 C T); (6169 11024374 T
C); (6170 11024388 C T); (6171 11024677 G C); (6172 11024697 A G); (6173 11024934 C A); (6174
11025139 A AT); (6175 11025143 A G); (6176 11025477 C T); (6177 11026543 A T); (6178 11026675 T
C); (6179 11027859 C T); (6180 11028026 G GA); (6181 11028289 T A); (6182 11028292 A
ATACAT); (6183 11028535 G A); (6184 11028693 T C); (6185 11028705 A C); (6186 11028986 A G); (6187
11029123 A AT); (6188 11029738 T G); (6189 11029890 T G); (6190 11030105 A G); (6191 11030559 A
G); (6192 11030844 G A); (6193 11030848 G A); (6194 11030848 GTA G); (6195 11030852 A G); (6196
11031848 A T); (6197 11031963 A G); (6198 11032694 A G); (6199 11032970 T C); (6200 11033172 T
G); (6201 11033921 A G); (6202 11033968 A G); (6203 11034076 T A); (6204 11034110 G GA); (6205
11034186 A G); (6206 11034219 C T); (6207 11034259 T C); (6208 11034313 A G); (6209 11034656 G
A); (6210 11034725 C G); (6211 11035055 T C); (6212 11035546 T C); (6213 11035746 C T); (6214
11035911 C A); (6215 11035970 CT C); (6216 11036101 C CAA); (6217 11036177 C T); (6218 11036353 A
T); (6219 11036443 T C); (6220 11036499 G A); (6221 11036522 C T); (6222 11036582 G A); (6223
11036675 A G); (6224 11036896 T C); (6225 11037674 T C); (6226 11037736 T C); (6227 11038393 A
T); (6228 11038514 A C); (6229 11038801 T G); (6230 11040589 CGAATGATGGAGAA C); (6231 11040756
T A); (6232 11040935 G GTTGTCAATATTTATTCTTGTT); (6233 11041227 G A); (6234 11041486 G
A); (6235 11041511 G A); (6236 11041735 T C); (6237 11041833 C T); (6238 11041892 T C); (6239
11042361 G A); (6240 11043633 A G); (6241 11044825 G A); (6242 11044936 C T); (6243 11045092 G
A); (6244 11045268 T TACTTACAGATCTTATGTA); (6245 11045901 C A); (6246 11045972 A T); (6247
11046146 C T); (6248 11046874 A G); (6249 11047350 A G); (6250 11047427 A G); (6251 11047785 A
AT); (6252 11047878 T A); (6253 11048007 C T); (6254 11048191 G A); (6255 11049022 G C); (6256
11049401 A G); (6257 11049497 A G); (6258 11049601 G A); (6259 11049667 T C); (6260 11049695 C
T); (6261 11049811 G A); (6262 11049921 T A); (6263 11049936 A AAAG); (6264 11049971 C A); (6265
11050240 T A); (6266 11050342 AT A); (6267 11050690 G A); (6268 11051362 C T); (6269 11051560 C
T); (6270 11052643 A T); (6271 11052741 A G); (6272 11052912 T G); (6273 11052989 C T); (6274
11053036 T A); (6275 11053102 C T); (6276 11053176 G A); (6277 11053277 T C); (6278 11053349 G
A); (6279 11053452 T C); (6280 11053648 C T); (6281 11053926 G C); (6282 11053982 A G); (6283
11054064 T C); (6284 11054159 G A); (6285 11054254 AAG A); (6286 11054585 C T); (6287 11054775 C
T); (6288 11054818 T C); (6289 11055217 T TA); (6290 11055254 A G); (6291 11055713 A T); (6292
11055735 G A); (6293 11056136 G A); (6294 11056502 G C); (6295 11056717 T C); (6296 11056754 A
G); (6297 11057102 GT G); (6298 11057157 C T); (6299 11057937 G C); (6300 11058057 G A); (6301
11058634 AC A); (6302 11058738 A T); (6303 11059118 C T); (6304 11059216 T G); (6305 11059243 A
G); (6306 11059334 G A); (6307 11059450 G T); (6308 11059836 C T); (6309 11059939 A C); (6310
11059943 T G); (6311 11060579 A AT); (6312 11061036 A T); (6313 11063547 T A); (6314 11064065 T
A); (6315 11064120 G C); (6316 11065185 C T); (6317 11065578 G A); (6318 11065822 G A); (6319
11066085 T C); (6320 11067270 A C); (6321 11067500 A G); (6322 11067974 T C); (6323 11069622 C
T); (6324 11069699 G GGGCA); (6325 11069782 G C); (6326 11070504 C T); (6327 11070883 C T); (6328
11070911 A G); (6329 11071345 T C); (6330 11072006 C T); (6331 11072269 A T); (6332 11072517 A
G); (6333 11073525 T G); (6334 11074398 A G); (6335 11074955 G A); (6336 11075092 C G); (6337
11075225 G A); (6338 11076726 G A); (6339 11077741 A G); (6340 11079616 C T); (6341 11084514 C
T); (6342 11084521 T C); (6343 11084566 A G); (6344 11084883 T C); (6345 11085982 G A); (6346
11086010 A G); (6347 11086058 G C); (6348 11086121 T G); (6349 11086312 C A); (6350 11086318 G
A); (6351 11086330 G A); (6352 11086335 T C); (6353 11086357 C T); (6354 11086367 G A); (6355
11086369 C A); (6356 11086382 C G); (6357 11086387 C T); (6358 11086406 C T); (6359 11086408 G
A); (6360 11086423 A G); (6361 11086512 G GTGCACATACAAAGATGATTCT); (6362 11086520 C A); (6363
11086524 T C); (6364 11086539 T C); (6365 11086559 C T); (6366 11086570 TA T); (6367 11086584 C
T); (6368 11086616 T C); (6369 11086650 G A); (6370 11086711 T C); (6371 11086729 G A); (6372
11086894 G A); (6373 11087216 A G); (6374 11087426 A C); (6375 11087452 A G); (6376 11087466 C
T); (6377 11087505 C T); (6378 11087861 A G); (6379 11087946 T G); (6380 11088045 G A); (6381
11088099 T C); (6382 11088222 T G); (6383 11088385 G T); (6384 11088454 G A); (6385 11088484 C
T); (6386 11088879 T C); (6387 11088904 T A); (6388 11089083 T C); (6389 11089298 C T); (6390
11089351 G A); (6391 11089529 C A); (6392 11089600 A G); (6393 11089784 T C); (6394 11089805 G
A); (6395 11089971 GGA G); (6396 11090507 G A); (6397 11090860 T C); (6398 11090866 C T); (6399
11090889 C T); (6400 11091364 T C); (6401 11091390 C T); (6402 11091414 C T); (6403 11091452 C
T); (6404 11091616 G A); (6405 11091662 A T); (6406 11091877 G A); (6407 11091907 G A); (6408
11092347 T A); (6409 11092425 TA T); (6410 11093414 C G); (6411 11094044 AGAAAAAG A); (6412
11094154 A G); (6413 11095022 G A); (6414 11095900 C T); (6415 11096228 C G); (6416 11096309 A
C); (6417 11096314 T A); (6418 11096609 C A); (6419 11096987 G C); (6420 11097150 GT G); (6421
11097262 A T); (6422 11097359 G A); (6423 11097488 C A); (6424 11098028 G T); (6425 11098217 T
G); (6426 11098253 T TA); (6427 11098658 G GT); (6428 11099085 G C); (6429 11099133 C A); (6430
11099670 C T); (6431 11099951 C T); (6432 11100122 C T); (6433 11100671 A G); (6434 11101252 G
C); (6435 11101331 T C); (6436 11102290 A C); (6437 11103535 A G); (6438 11103900 G T); (6439
11104265 G A); (6440 11104908 A G); (6441 11105794 G A); (6442 11106330 A G); (6443 11106452 C
T); (6444 11106514 G A); (6445 11106634 T C); (6446 11107177 T C); (6447 11107255 T G); (6448
11107554 A C); (6449 11108202 A G); (6450 11108291 C T); (6451 11108317 C G); (6452 11108796 A
G); (6453 11108834 T C); (6454 11108942 A G); (6455 11108956 A G); (6456 11109021 T C); (6457
11109262 T G); (6458 11110838 T A); (6459 11111038 C A); (6460 11111514 C T); (6461 11112122 A
AAAT); (6462 11112303 AC A); (6463 11112486 T TA); (6464 11112600 T TATA); (6465 11113539 T
G); (6466 11113671 A C); (6467 11114474 T C); (6468 11115052 G A); (6469 11115447 A G); (6470
11115560 T C); (6471 11115984 G A); (6472 11116096 C T); (6473 11116685 A G); (6474 11117596 T
C); (6475 11117987 G A); (6476 11118382 T A); (6477 11118454 C T); (6478 11118828 T G); (6479
11118836 C T); (6480 11118856 T C); (6481 11118909 T C); (6482 11118926 G A); (6483 11118950 G
A); (6484 11118956 G A); (6485 11118975 A T); (6486 11118983 C T); (6487 11119199 C T); (6488
11120642 AT A); (6489 11121216 G A); (6490 11121276 A G); (6491 11121860 A G); (6492 11122319 TG
T); (6493 11124348 C T); (6494 11124508 G A); (6495 11124620 G A); (6496 11124636 C G); (6497
11124693 G C); (6498 11124694 C G); (6499 11124717 C T); (6500 11124958 T TCA); (6501 11126821 A
T); (6502 11126885 A C); (6503 11127087 T G); (6504 11127141 A G); (6505 11127830 G A); (6506
11127979 G GT); (6507 11128159 A T); (6508 11128485 C T); (6509 11128685 T C); (6510 11129873 T
TA); (6511 11130278 GA TG); (6512 11130280 T A); (6513 11130317 TA T); (6514 11130459 C A); (6515
11130562 A AATATAT); (6516 11130687 A G); (6517 11130840 CA C); (6518 11131707 T TA); (6519
11132024 TTTC T); (6520 11132028 T C); (6521 11132076 A G); (6522 11132093 G A); (6523 11132131 A
G); (6524 11132143 C T); (6525 11132204 G A); (6526 11132225 G A); (6527 11132814 T C); (6528
11133003 A G); (6529 11133039 T A); (6530 11133087 G A); (6531 11133097 A T); (6532 11133180 A
G); (6533 11133286 G A); (6534 11133358 G GGTTCTGGGCGTGACCAGTCATGCCCA); (6535 11133558 G
A); (6536 11133654 T A); (6537 11133782 C T); (6538 11133825 C T); (6539 11134058 A C); (6540
11134317 G A); (6541 11134977 C CA); (6542 11137543 T TA); (6543 11137599 T C); (6544 11137675 TA
T); (6545 11138353 A T); (6546 11138354 A T); (6547 11138847 A G); (6548 11139091 TA T); (6549
11139536 G A); (6550 11140109 A AT); (6551 11141067 T C); (6552 11141184 AT A); (6553 11141274 T
G); (6554 11141557 A T); (6555 11141650 C A); (6556 11143173 A G); (6557 11145012 T G); (6558
11145535 C CA); (6559 11145573 A G); (6560 11145655 A G); (6561 11145801 G A); (6562 11145898 C
A); (6563 11146173 C T); (6564 11146297 C T); (6565 11146372 T C); (6566 11146443 G T); (6567
11146447 G A); (6568 11146517 T C); (6569 11146598 C G); (6570 11146643 C A); (6571 11146719 A
T); (6572 11146812 C T); (6573 11146821 C A); (6574 11146828 G C); (6575 11146837 A C); (6576
11146873 T A); (6577 11146891 T C); (6578 11147195 A T); (6579 11147319 A G); (6580 11147402 G
C); (6581 11147487 T C); (6582 11147713 C T); (6583 11147825 T A); (6584 11147835 T A); (6585
11147837 A T); (6586 11147844 AATAC A); (6587 11147848 A T); (6588 11147936 T G); (6589 11147946
A C); (6590 11147948 CT C); (6591 11147968 T C); (6592 11147989 T C); (6593 11148013 G C); (6594
11148105 T C); (6595 11148212 C CTA); (6596 11148304 T A); (6597 11148347 CTCA C); (6598 11148394
C T); (6599 11148416 C T); (6600 11148454 T A); (6601 11148546 T C); (6602 11148557 C T); (6603
11148692 C T); (6604 11148707 G T); (6605 11148713 A G); (6606 11148740 A G); (6607 11148780 G
C); (6608 11148785 A T); (6609 11148789 A G); (6610 11148839 C A); (6611 11148853 A G); (6612
11148880 T G); (6613 11148894 C G); (6614 11149005 GA G); (6615 11149023 A G); (6616 11149228 C
T); (6617 11149324 A G); (6618 11149389 CA C); (6619 11149435 A T); (6620 11149472 T C); (6621
11149563 G A); (6622 11149564 G T); (6623 11149634 A ATAAAT); (6624 11149724 T TCATG); (6625
11149807 G A); (6626 11149853 A T); (6627 11149981 A G); (6628 11150069 C A); (6629 11150082 C
T); (6630 11150134 G A); (6631 11150142 A ATCT); (6632 11150220 A C); (6633 11150340 C T); (6634
11150517 G A); (6635 11150552 CAT C); (6636 11150771 G C); (6637 11150939 A C); (6638 11151016 A
T); (6639 11151208 T A); (6640 11151332 G A); (6641 11151369 T C); (6642 11151593 C A); (6643
11151594 A C); (6644 11151667 T TTTTAATAATA); (6645 11151845 C T); (6646 11151857 A AT); (6647
11151953 A T); (6648 11152013 C T); (6649 11152017 A G); (6650 11152020 C A); (6651 11152056 G
T); (6652 11152089 A AT); (6653 11152138 A G); (6654 11152149 T C); (6655 11152197 G A); (6656
11153178 T A); (6657 11153255 T A); (6658 11153306 T TGGATACTGG); (6659 11153364 GAA G); (6660
11153406 C T); (6661 11153796 A ATTG); (6662 11153886 C T); (6663 11153911 C T); (6664 11153925 G
T); (6665 11153950 C A); (6666 11154009 T C); (6667 11154012 A T); (6668 11154078 C G); (6669
11154182 A T); (6670 11154236 AT A); (6671 11154362 CA C); (6672 11154603 C G); (6673 11154670 C
G); (6674 11154791 G A); (6675 11154849 T TA); (6676 11154881 C T); (6677 11154941 A C); (6678
11154988 C T); (6679 11155153 C A); (6680 11155231 C T); (6681 11155282 C T); (6682 11155292 G
GA); (6683 11155556 GTCTT G); (6684 11155568 G A); (6685 11155571 G A); (6686 11155576 A C); (6687
11155582 T G); (6688 11155643 A G); (6689 11155719 G T); (6690 11155731 G A); (6691 11155736 T
A); (6692 11155746 T C); (6693 11155752 T C); (6694 11155794 T TTA); (6695 11155804 T C); (6696
11155883 C T); (6697 11155930 T G); (6698 11156053 T A); (6699 11156054 T C); (6700 11156058 A
T); (6701 11156067 T C); (6702 11156231 C T); (6703 11156273 T C); (6704 11156298 T G); (6705
11156305 A T); (6706 11156311 A T); (6707 11156456 G A); (6708 11156600 G T); (6709 11156748 G
A); (6710 11156903 T C); (6711 11156939 G A); (6712 11159380 A G); (6713 11159407 T C); (6714
11159410 A G); (6715 11159440 G A); (6716 11159575 A G); (6717 11159949 T C); (6718 11160688 C
T); (6719 11160690 T C); (6720 11160759 C T); (6721 11160774 G T); (6722 11160777 T C); (6723
11160789 A G); (6724 11160798 G A); (6725 11160810 C T); (6726 11160813 C T); (6727 11161558 T
A); (6728 11161587 G A); (6729 11161591 T C); (6730 11161756 G A); (6731 11161760 A G); (6732
11161761 G A); (6733 11162729 G A); (6734 11162738 G A); (6735 11162792 A G); (6736 11162804 A
G); (6737 11162837 A G); (6738 11162840 G A); (6739 11164767 G A); (6740 11165057 T C); (6741
11165061 T A); (6742 11165066 G A); (6743 11165343 C T); (6744 11165409 T C); (6745 11165480 A
G); (6746 11165502 A G); (6747 11165951 T C); (6748 11166007 G C); (6749 11166017 C T); (6750
11167428 T C); (6751 11167431 C T); (6752 11167452 C T); (6753 11168140 T C); (6754 11168169 G
A); (6755 11168342 T G); (6756 11168574 T A); (6757 11168736 A T); (6758 11168801 A ATT); (6759
11168940 C T); (6760 11168985 C T); (6761 11169070 AACGACAT A); (6762 11169073 A G); (6763
11169199 A G); (6764 11169253 G A); (6765 11169265 T G); (6766 11169412 CAA C); (6767 11169521 G
A); (6768 11169558 T C); (6769 11169602 C A); (6770 11169671 T C); (6771 11169791 G A); (6772
11169795 T A); (6773 11169891 T TA); (6774 11170075 C T); (6775 11170516 T C); (6776 11170520 A
G); (6777 11170749 T C); (6778 11170805 T G); (6779 11170847 C T); (6780 11171094 G A); (6781
11171124 T C); (6782 11171143 T C); (6783 11171315 C T); (6784 11171364 C T); (6785 11171396 T
C); (6786 11171419 G A); (6787 11171424 T C); (6788 11171439 G A); (6789 11171440 T C); (6790
11171441 G A); (6791 11171452 C T); (6792 11171515 T A); (6793 11171520 G
GCCACACCCCACGCGACCCGCA); (6794 11171528 T C); (6795 11171563 T C); (6796 11171571 G A); (6797
11171573 G A); (6798 11171622 GATCCGCGCCAGATCTC G); (6799 11171624 A G); (6800 11171880 G
A); (6801 11171899 A G); (6802 11171999 C T); (6803 11172063 C T); (6804 11172126 T C); (6805
11172217 C T); (6806 11172248 A G); (6807 11172266 T C); (6808 11172280 T C); (6809 11172340 A
C); (6810 11172412 G A); (6811 11172522 G T); (6812 11172582 T C); (6813 11172625 A G); (6814
11172886 G A); (6815 11172893 T C); (6816 11172922 C T); (6817 11172923 A G); (6818 11172925 C
T); (6819 11173041 A G); (6820 11173064 G A); (6821 11173128 A G); (6822 11173238 T C); (6823
11173249 A G); (6824 11173321 G A); (6825 11173368 A G); (6826 11173382 G A); (6827 11173390 C
T); (6828 11173407 A G); (6829 11173417 C T); (6830 11173451 G A); (6831 11173610 A C); (6832
11173632 C A); (6833 11173708 G A); (6834 11173712 A C); (6835 11173737 A T); (6836 11173757 G
A); (6837 11173760 T C); (6838 11173774 T C); (6839 11173794 G A); (6840 11173892 C T); (6841
11173929 C T); (6842 11173997 AC A); (6843 11174060 T C); (6844 11174123 C T); (6845 11174208 C
T); (6846 11174215 T C); (6847 11174230 T G); (6848 11174265 C T); (6849 11174324 A T); (6850
11174393 A G); (6851 11174396 T C); (6852 11174433 A G); (6853 11174486 C T); (6854 11174500 G
A); (6855 11174526 G A); (6856 11174549 T C); (6857 11174568 A C); (6858 11174585 C T); (6859
11174587 G C); (6860 11174640 G C); (6861 11174809 C G); (6862 11174900 C A); (6863 11175056 T
TAA); (6864 11175346 G T); (6865 11175366 T G); (6866 11175708 T A); (6867 11175716 C T); (6868
11175980 CTTGATAA C); (6869 11176082 T TA); (6870 11176122 C T); (6871 11176139 T C); (6872
11176177 A G); (6873 11176198 T C); (6874 11176221 G T); (6875 11176229 T C); (6876 11176235 G
A); (6877 11176359 C T); (6878 11176376 T C); (6879 11176381 C T); (6880 11176426 T C); (6881
11176451 TA T); (6882 11176724 A G); (6883 11176727 C T); (6884 11176775 G A); (6885 11176795 A
C); (6886 11176820 A T); (6887 11176868 G T); (6888 11179975 G A); (6889 11182427 G C); (6890
11184301 C G); (6891 11187456 C T); (6892 11187731 T C); (6893 11189940 G A); (6894 11190913 G
T); (6895 11192205 A G); (6896 11192623 T C); (6897 11193267 G T); (6898 11194149 T C); (6899
11194267 A G); (6900 11196000 T C); (6901 11199054 C T); (6902 11199997 C T); (6903 11200230 C
T); (6904 11204201 C G); (6905 11204316 C G); (6906 11205960 C T); (6907 11206012 T C); (6908
11206255 C A); (6909 11206969 T C); (6910 11208285 C T); (6911 11208792 C A); (6912 11213151 G
T); (6913 11213923 T G); (6914 11214031 T C); (6915 11214045 C A); (6916 11214613 G A); (6917
11222229 CT C); (6918 11222989 T TTA); (6919 11223236 A T); (6920 11224654 G T); (6921 11232861 C
T); (6922 11233265 ATTTG A); (6923 11235140 T G); (6924 11237623 G A); (6925 11238382 A
AGAATT); (6926 11238602 C T); (6927 11239189 AT A); (6928 11240062 C CAATT); (6929 11245425 G
A); (6930 11248668 G A); (6931 11250396 C T); (6932 11259504 C A); (6933 11259746 C T); (6934
11262160 G A); (6935 11262504 C T); (6936 11263748 G C); (6937 11264607 G C); (6938 11264723 G
A); (6939 11265269 C T); (6940 11266531 A G); (6941 11268476 CT C); (6942 11272869 C T); (6943
11273636 G T); (6944 11275077 T A); (6945 11275996 A T); (6946 11276441 C T); (6947 11276682 A
G); (6948 11276972 C T); (6949 11284233 G C); (6950 11285211 C G); (6951 11292810 T C); (6952
11294172 C T); (6953 11295067 G A); (6954 11296445 G A); (6955 11298325 ATT A); (6956 11300916 C
A); (6957 11304912 A C); (6958 11308169 G A); (6959 11310319 C T); (6960 11313483 C T); (6961
11313631 C T); (6962 11317267 G A); (6963 11317499 G A); (6964 11317523 T C); (6965 11326616 G
A); (6966 11327699 G A); (6967 11328788 G A); (6968 11331497 AT A); (6969 11344519 C T); (6970
11349758 G A); (6971 11350811 G A); (6972 11353236 C T); (6973 11356557 G A); (6974 11360321 C
T); (6975 11364893 C CT); (6976 11365604 C A); (6977 11366044 A C); (6978 11366146 C T); (6979
11367015 GT G); (6980 11367278 G A); (6981 11373930 T G); (6982 11375730 T A); (6983 11377911 GT
G); (6984 11382090 A G); (6985 11382733 C T); (6986 11383132 C T); (6987 11384237 A C); (6988
11386745 T C); (6989 11388076 A T); (6990 11388762 G C); (6991 11390287 C T); (6992 11391526 T
C); (6993 11393111 G A); (6994 11395102 A G); (6995 11395350 A G); (6996 11395943 G A); (6997
11396356 T C); (6998 11397964 T G); (6999 11398374 C T); (7000 11398544 G A); (7001 11398617 C
T); (7002 11400424 T A); (7003 11400735 A G); (7004 11401048 A C); (7005 11401654 A G); (7006
11401763 C G); (7007 11403394 A G); (7008 11407301 G A); (7009 11409203 G A); (7010 11410756 C
G); (7011 11411203 T C); (7012 11411595 A G); (7013 11411824 A G); (7014 11412400 CATAT C); (7015
11412602 A C); (7016 11412623 GACTCTTGTTCCCAAACCTAA G); (7017 11415980 G A); (7018 11416627
AT A); (7019 11418901 G A); (7020 11419488 T C); (7021 11421713 C T); (7022 11421970 C G); (7023
11422046 G C); (7024 11427270 T C); (7025 11431446 C A); (7026 11434473 C T); (7027 11436127 A
G); (7028 11436183 C CT); (7029 11440881 T C); (7030 11443551 T TA); (7031 11447065 C T); (7032
11450464 C T); (7033 11451021 C T); (7034 11451149 G A); (7035 11451834 G A); (7036 11452446 T
C); (7037 11456001 ATTT A); (7038 11461621 CA C); (7039 11462713 G C); (7040 11462743 C A); (7041
11462761 C T); (7042 11463273 G A); (7043 11465948 G A); (7044 11468213 A G); (7045 11469344 C
T); (7046 11470514 T C); (7047 11473346 T C); (7048 11475022 C T); (7049 11476005 G T); (7050
11477811 T C); (7051 11477851 G A); (7052 11480220 A C); (7053 11481113 AGTTAT A); (7054
11484242 A C); (7055 11485290 A G); (7056 11485836 A T); (7057 11485996 A G); (7058 11487159 C
T); (7059 11491950 A T); (7060 11494622 C A); (7061 11495213 G C); (7062 11497739 G A); (7063
11502363 C T); (7064 11505998 T C); (7065 11506265 T C); (7066 11508191 A G); (7067 11511062 T
TACAACA); (7068 11511305 C A); (7069 11511310 C A); (7070 11512366 T A); (7071 11513744 A
C); (7072 11514035 AT A); (7073 11514532 G C); (7074 11514931 T C); (7075 11516365 C T); (7076
11517130 C T); (7077 11517709 C G); (7078 11520201 A G); (7079 11520584 C T); (7080 11522522 A
G); (7081 11524612 G A); (7082 11524730 C T); (7083 11525304 G A); (7084 11525635 C T); (7085
11526549 A G); (7086 11527556 C A); (7087 11527680 G A); (7088 11527856 C T); (7089 11528229 T
C); (7090 11528259 T C); (7091 11530067 GT G); (7092 11530107 T C); (7093 11534016 T G); (7094
11536596 TA T); (7095 11541271 A G); (7096 11542888 TA T); (7097 11543667 C G); (7098 11544531 C
G); (7099 11545884 T C); (7100 11546074 G C); (7101 11546920 G A); (7102 11550920 C T); (7103
11552781 G T); (7104 11561120 C T); (7105 11561183 G A); (7106 11567081 G A); (7107 11567209 C
T); (7108 11567635 GC G); (7109 11568082 G A); (7110 11568600 G A); (7111 11568858 C G); (7112
11569054 G A); (7113 11570697 C T); (7114 11573565 C T); (7115 11579396 C T); (7116 11580900 C
T); (7117 11582112 C T); (7118 11584309 C T); (7119 11585407 G A); (7120 11586054 T C); (7121
11587470 A G); (7122 11589839 T G); (7123 11589981 G A); (7124 11593127 G C); (7125 11597777 C
T); (7126 11599987 T C); (7127 11600050 A G); (7128 11600122 C T); (7129 11600146 C A); (7130
11600153 C T); (7131 11600154 T C); (7132 11600165 G T); (7133 11600166 A C); (7134 11600172 T
TTA); (7135 11600175 A G); (7136 11600177 G A); (7137 11601009 T A); (7138 11601033 CA C); (7139
11601045 C T); (7140 11601415 C T); (7141 11601457 G A); (7142 11602226 G A); (7143 11602254 A
G); (7144 11603012 T C); (7145 11603673 C T); (7146 11603676 A G); (7147 11603682 C T); (7148
11603708 C A); (7149 11603714 C T); (7150 11603730 T TG); (7151 11604060 GC G); (7152 11604123 A
C); (7153 11604127 G A); (7154 11604762 A T); (7155 11604773 C T); (7156 11604810 C G); (7157
11608430 T C); (7158 11608734 C A); (7159 11610300 C T); (7160 11610964 C T); (7161 11614303 T
G); (7162 11615932 TTTGGTACTGGA T); (7163 11616028 G A); (7164 11620381 C T); (7165 11621400 A
G); (7166 11622176 C T); (7167 11623132 A G); (7168 11623588 A G); (7169 11623687 C T); (7170
11624220 TAAA T); (7171 11624823 G A); (7172 11625018 G A); (7173 11625032 T G); (7174 11626313 C
T); (7175 11626459 T C); (7176 11626763 T C); (7177 11628798 C G); (7178 11629519 A C); (7179
11629543 A G); (7180 11629676 A G); (7181 11629766 A G); (7182 11629771 A G); (7183 11630102 C
G); (7184 11630148 A G); (7185 11630321 T C); (7186 11630465 A G); (7187 11630744 T A); (7188
11631000 C A); (7189 11631960 T C); (7190 11632183 T G); (7191 11632730 T C); (7192 11632778 T
C); (7193 11633022 G A); (7194 11633068 C G); (7195 11633151 C T); (7196 11633333 T C); (7197
11633381 T C); (7198 11633403 T C); (7199 11633467 T C); (7200 11633645 T G); (7201 11635262 T
C); (7202 11636264 A G); (7203 11636485 G A); (7204 11636696 C A); (7205 11637063 C A); (7206
11638039 A T); (7207 11638076 C T); (7208 11638141 T C); (7209 11638176 A T); (7210 11638195 A
G); (7211 11638269 C T); (7212 11638371 G A); (7213 11638393 A T); (7214 11638459 A G); (7215
11638562 C A); (7216 11641302 C T); (7217 11641835 G A); (7218 11641876 T C); (7219 11641930 C
T); (7220 11642048 T A); (7221 11642070 T C); (7222 11642105 G A); (7223 11642110 A G); (7224
11642118 C T); (7225 11642241 C T); (7226 11642255 T G); (7227 11642307 T C); (7228 11642948 C
T); (7229 11642963 C T); (7230 11643009 C T); (7231 11643040 A G); (7232 11643055 G A); (7233
11643530 A T); (7234 11643681 C A); (7235 11643841 C A); (7236 11644045 A C); (7237 11644053 A
G); (7238 11644056 C A); (7239 11644069 T C); (7240 11644074 A G); (7241 11644081 G A); (7242
11644338 A T); (7243 11644359 T C); (7244 11644433 C T); (7245 11644692 C A); (7246 11644720 C
A); (7247 11644723 T C); (7248 11645214 C T); (7249 11645234 C A); (7250 11645235 T A); (7251
11645236 C A); (7252 11645243 A G); (7253 11645251 A G); (7254 11645295 A T); (7255 11645315 C
T); (7256 11645384 C T); (7257 11645405 T C); (7258 11645422 C T); (7259 11645492 T C); (7260
11645497 A G); (7261 11645498 A G); (7262 11645505 T C); (7263 11645512 T C); (7264 11645535 G
T); (7265 11645537 A C); (7266 11645538 C T); (7267 11645540 C T); (7268 11648316 G A); (7269
11648474 G C); (7270 11648663 G A); (7271 11648854 A G); (7272 11650581 G T); (7273 11650583 C
CTT); (7274 11650940 G A); (7275 11650956 G A); (7276 11651098 A G); (7277 11651245 T C); (7278
11651816 T G); (7279 11652054 C T); (7280 11652129 G A); (7281 11652168 G A); (7282 11652246 A
G); (7283 11654007 T G); (7284 11654442 A G); (7285 11658540 C T); (7286 11659153 GTAA G); (7287
11659166 G C); (7288 11659284 C T); (7289 11659295 T C); (7290 11659401 A C); (7291 11659884 G
A); (7292 11659981 T C); (7293 11660125 T TA); (7294 11660160 G A); (7295 11660346 G A); (7296
11660634 C A); (7297 11660707 A C); (7298 11660709 A T); (7299 11660711 T C); (7300 11661020 C
A); (7301 11661027 CCTAA C); (7302 11661102 G T); (7303 11661184 A G); (7304 11661241 T C); (7305
11661460 C T); (7306 11661562 G C); (7307 11662329 T TA); (7308 11663370 A G); (7309 11663411 C
CATATATATAT); (7310 11663630 C T); (7311 11664033 A AT); (7312 11664304 G A); (7313 11664378 T
G); (7314 11664448 T C); (7315 11664792 A C); (7316 11664797 C G); (7317 11664802 C G); (7318
11666137 G A); (7319 11666699 C G); (7320 11666832 T C); (7321 11666856 GA G); (7322 11666946 A
G); (7323 11666950 C G); (7324 11667153 G GA); (7325 11667358 T A); (7326 11667567 C T); (7327
11667898 G A); (7328 11668395 C T); (7329 11668583 C T); (7330 11668851 G C); (7331 11669657 C
T); (7332 11669715 C T); (7333 11669766 A AC); (7334 11669888 A AC); (7335 11670061 G A); (7336
11670130 A G); (7337 11670141 G A); (7338 11670176 A G); (7339 11670284 C T); (7340 11670356 G
A); (7341 11670368 C A); (7342 11670411 A T); (7343 11670655 A G); (7344 11670859 G A); (7345
11670879 A G); (7346 11670934 T C); (7347 11670999 C A); (7348 11672014 A G); (7349 11672887 A
G); (7350 11674169 C T); (7351 11674193 G A); (7352 11674275 C G); (7353 11674370 T C); (7354
11674462 G A); (7355 11674549 C T); (7356 11674790 G A); (7357 11674795 T G); (7358 11674997 T
C); (7359 11675067 T C); (7360 11675275 C T); (7361 11675291 G
GGCGTGGGAGCCCACGCTTGGA); (7362 11675303 A G); (7363 11675686 T C); (7364 11675729 T
C); (7365 11675802 C T); (7366 11675888 A C); (7367 11675921 G A); (7368 11675965 A G); (7369
11676103 T C); (7370 11676118 G A); (7371 11676534 A C); (7372 11676838 T G); (7373 11676877 A
G); (7374 11677592 A G); (7375 11677618 TAAAACCTTGTTTATC T); (7376 11677628 G A); (7377
11677949 T G); (7378 11678206 T G); (7379 11678412 G A); (7380 11678421 T C); (7381 11678430 C
T); (7382 11678787 A G); (7383 11678894 T G); (7384 11678903 G A); (7385 11679053 C T); (7386
11679567 T G); (7387 11679875 A G); (7388 11680033 T A); (7389 11680137 A G); (7390 11680392 C
T); (7391 11680467 A G); (7392 11680504 G A); (7393 11680625 G A); (7394 11680814 G C); (7395
11680933 C T); (7396 11680964 A C); (7397 11681198 T C); (7398 11681222 A AT); (7399 11681298 G
A); (7400 11681968 G A); (7401 11682012 C T); (7402 11682058 C A); (7403 11682141 T G); (7404
11682755 G T); (7405 11683012 C T); (7406 11683762 C T); (7407 11684349 C T); (7408 11684389 G
A); (7409 11684570 G A); (7410 11684861 C T); (7411 11685079 A C); (7412 11685125 T C); (7413
11685315 G A); (7414 11685422 A G); (7415 11685881 T C); (7416 11685981 T C); (7417 11685993 G
C); (7418 11686048 G A); (7419 11686253 T G); (7420 11686284 A G); (7421 11686369 T C); (7422
11686627 C T); (7423 11687136 T C); (7424 11687205 G T); (7425 11687219 T A); (7426 11687388 C
T); (7427 11687406 T TA); (7428 11687611 T TG); (7429 11687837 T C); (7430 11687919 G A); (7431
11688006 T C); (7432 11688880 T C); (7433 11689220 C A); (7434 11689274 T C); (7435 11689708 A
C); (7436 11689848 G A); (7437 11689944 G C); (7438 11690314 T G); (7439 11690422 A G); (7440
11690837 A T); (7441 11691166 C A); (7442 11691394 A T); (7443 11691459 C A); (7444 11693318 T
A); (7445 11693326 C T); (7446 11693380 C A); (7447 11693405 C A); (7448 11693506 A G); (7449
11693740 C T); (7450 11693748 G T); (7451 11694299 T C); (7452 11694321 A T); (7453 11694409 T
G); (7454 11694466 AT A); (7455 11694507 A C); (7456 11694554 T C); (7457 11694615 A C); (7458
11694667 G A); (7459 11694691 G T); (7460 11694848 A G); (7461 11694955 T C); (7462 11694983 A
G); (7463 11696317 C T); (7464 11696318 A G); (7465 11696455 T C); (7466 11696509 C T); (7467
11696818 G A); (7468 11696925 A C); (7469 11697369 CAA C); (7470 11697555 A G); (7471 11697572 G
A); (7472 11697631 C G); (7473 11697728 C A); (7474 11697876 A G); (7475 11698709 T C); (7476
11698861 T C); (7477 11699469 T A); (7478 11699476 A C); (7479 11699714 G C); (7480 11700099 T
A); (7481 11700132 G A); (7482 11700174 G A); (7483 11700415 A C); (7484 11700504 C T); (7485
11700520 C T); (7486 11700723 T C); (7487 11700815 C G); (7488 11700892 A C); (7489 11700991 C
A); (7490 11701112 G A); (7491 11701962 G A); (7492 11702426 G A); (7493 11702615 T C); (7494
11702847 A AC); (7495 11702853 A T); (7496 11702914 A G); (7497 11703409 A G); (7498 11703544 A
G); (7499 11703653 G T); (7500 11703689 T G); (7501 11703971 A G); (7502 11704070 C G); (7503
11704532 T C); (7504 11704789 G A); (7505 11705003 A G); (7506 11705299 G A); (7507 11705530 C
T); (7508 11705567 G A); (7509 11705693 A C); (7510 11705960 A G); (7511 11706081 C T); (7512
11706126 T C); (7513 11706683 CT C); (7514 11706918 G A); (7515 11707110 T C); (7516 11707119 G
A); (7517 11707139 T A); (7518 11707501 G A); (7519 11707582 T C); (7520 11707774 A T); (7521
11707827 T C); (7522 11708050 C T); (7523 11708113 G A); (7524 11708921 A C); (7525 11709049 T
G); (7526 11709310 G A); (7527 11709334 C T); (7528 11709497 A G); (7529 11709550 A G); (7530
11709562 T C); (7531 11709648 G C); (7532 11710373 C T); (7533 11710384 T C); (7534 11710395 G
GA); (7535 11710450 G A); (7536 11710708 AT A); (7537 11711321 A G); (7538 11711695 A G); (7539
11711706 A G); (7540 11711786 A T); (7541 11711859 G A); (7542 11712940 A G); (7543 11713114 T
C); (7544 11713137 A C); (7545 11713881 A G); (7546 11714530 A G); (7547 11714665 T C); (7548
11714676 A G); (7549 11716291 G A); (7550 11716813 A G); (7551 11717351 G A); (7552 11717594 A
G); (7553 11717742 T C); (7554 11718097 T A); (7555 11718098 T C); (7556 11718449 G A); (7557
11718587 T C); (7558 11718685 A G); (7559 11718793 A G); (7560 11719143 C A); (7561 11719223 A
T); (7562 11719426 G A); (7563 11719725 C T); (7564 11720039 T C); (7565 11720385 C T); (7566
11720393 A C); (7567 11720400 A G); (7568 11720405 T A); (7569 11720421 G A); (7570 11720426 A
G); (7571 11720440 C A); (7572 11720443 A T); (7573 11720742 T A); (7574 11720994 A G); (7575
11721038 G C); (7576 11721090 T C); (7577 11721097 C T); (7578 11721157 A G); (7579 11721478 T
C); (7580 11721480 G A); (7581 11722085 C T); (7582 11722450 TA T); (7583 11722685 A AT); (7584
11722733 A G); (7585 11722973 T C); (7586 11723190 G GA); (7587 11723305 AT A); (7588 11723413 T
TA); (7589 11723552 T A); (7590 11723955 T G); (7591 11724337 T A); (7592 11724513 C T); (7593
11724626 C A); (7594 11724752 G T); (7595 11724775 A G); (7596 11724957 G A); (7597 11725003 G
A); (7598 11725005 A G); (7599 11725007 G A); (7600 11725089 A G); (7601 11725106 G A); (7602
11725116 A C); (7603 11725151 C T); (7604 11725254 A G); (7605 11725303 C T); (7606 11725331 A
G); (7607 11725376 G A); (7608 11725837 A G); (7609 11726075 G A); (7610 11726138 C T); (7611
11726334 G A); (7612 11726450 G A); (7613 11726937 A G); (7614 11727255 A G); (7615 11727296 C
A); (7616 11727317 G A); (7617 11727438 T C); (7618 11727852 A C); (7619 11727972 A C); (7620
11728152 G A); (7621 11728178 G A); (7622 11728419 G GT); (7623 11728743 T C); (7624 11728802 C
G); (7625 11729936 G A); (7626 11729939 G A); (7627 11730242 T G); (7628 11730302 G C); (7629
11730444 C A); (7630 11730585 T C); (7631 11730733 A G); (7632 11730912 C T); (7633 11732528 G
T); (7634 11734841 G A); (7635 11735173 C T); (7636 11735562 A G); (7637 11735599 A G); (7638
11735908 A G); (7639 11735934 A C); (7640 11735955 G A); (7641 11736203 C A); (7642 11736388 T
C); (7643 11736402 A T); (7644 11736747 T C); (7645 11736753 C T); (7646 11736758 C G); (7647
11736861 T G); (7648 11737406 C T); (7649 11737500 C T); (7650 11737591 T C); (7651 11737825 A
C); (7652 11738056 T C); (7653 11738066 A G); (7654 11738277 G C); (7655 11738367 C T); (7656
11738497 A G); (7657 11738923 C T); (7658 11739491 C T); (7659 11739567 G A); (7660 11739641 T
C); (7661 11739690 A G); (7662 11739731 G A); (7663 11739830 G A); (7664 11739847 T C); (7665
11739909 A C); (7666 11740506 T C); (7667 11741011 G A); (7668 11741057 G A); (7669 11741203 G
A); (7670 11741952 T A); (7671 11742099 A G); (7672 11743027 A G); (7673 11743180 G A); (7674
11743260 A T); (7675 11743276 A AG); (7676 11743336 A G); (7677 11743411 T G); (7678 11743478 G
T); (7679 11744724 A G); (7680 11744941 A T); (7681 11745361 G A); (7682 11745363 TG T); (7683
11745794 G A); (7684 11745837 A G); (7685 11746342 C G); (7686 11746461 C A); (7687 11746482 A
G); (7688 11746574 T C); (7689 11746701 C A); (7690 11746974 G T); (7691 11747030 A G); (7692
11747270 T C); (7693 11747337 A G); (7694 11747931 C T); (7695 11749533 T TTA); (7696 11750787 T
G); (7697 11751088 G A); (7698 11751178 G A); (7699 11751561 T G); (7700 11753273 GA G); (7701
11754390 G T); (7702 11754485 G A); (7703 11754779 T TA); (7704 11754905 C T); (7705 11755092 A
C); (7706 11755834 T A); (7707 11756525 C CA); (7708 11756585 T G); (7709 11756714 C G); (7710
11756905 T C); (7711 11757262 T C); (7712 11757439 G A); (7713 11757467 C T); (7714 11757552 C
G); (7715 11757764 C T); (7716 11758021 G A); (7717 11758228 A G); (7718 11758741 G A); (7719
11758796 A G); (7720 11758917 A G); (7721 11758927 C T); (7722 11759081 G A); (7723 11759892 C
T); (7724 11759914 C A); (7725 11764496 G A); (7726 11764509 C T); (7727 11764511 G A); (7728
11764546 T C); (7729 11764556 C T); (7730 11764591 C T); (7731 11764601 T G); (7732 11764607 C
T); (7733 11764631 C A); (7734 11765476 GT G); (7735 11766804 A G); (7736 11766885 A G); (7737
11767134 C T); (7738 11767175 G A); (7739 11767725 T A); (7740 11768103 A C); (7741 11768439 C
T); (7742 11768624 TG T); (7743 11768712 G A); (7744 11768724 G A); (7745 11770286 A C); (7746
11770396 A C); (7747 11771471 C T); (7748 11771502 G C); (7749 11771788 T C); (7750 11772024 T
C); (7751 11772413 T C); (7752 11772445 T C); (7753 11772481 G A); (7754 11772653 G T); (7755
11772917 T C); (7756 11773100 A G); (7757 11773210 A G); (7758 11773807 G A); (7759 11774127 G
A); (7760 11774166 C T); (7761 11774168 C A); (7762 11774223 A T); (7763 11774650 C A); (7764
11775375 C T); (7765 11775384 C A); (7766 11775487 T C); (7767 11775508 G A); (7768 11776059 T
C); (7769 11776520 G A); (7770 11776566 T C); (7771 11782644 G T); (7772 11782666 A G); (7773
11786170 C T); (7774 11786171 T G); (7775 11787095 T C); (7776 11789579 T C); (7777 11789765 C
T); (7778 11789792 C G); (7779 11789866 T A); (7780 11789886 T A); (7781 11790101 G A); (7782
11791082 G A); (7783 11791114 T A); (7784 11793134 C T); (7785 11793137 A G); (7786 11793757 CA
C); (7787 11794154 C T); (7788 11794187 G A); (7789 11794481 A G); (7790 11794494 A C); (7791
11794527 A T); (7792 11794575 T C); (7793 11794580 T C); (7794 11794656 C T); (7795 11795019 G
A); (7796 11795048 T C); (7797 11795812 T C); (7798 11795849 G A); (7799 11795930 T C); (7800
11795939 A G); (7801 11796330 G C); (7802 11796402 G C); (7803 11796486 C T); (7804 11796513 C
T); (7805 11796562 G A); (7806 11796659 G A); (7807 11796697 T A); (7808 11796753 C T); (7809
11796793 G T); (7810 11796798 T C); (7811 11796874 T C); (7812 11797282 G C); (7813 11797297 T
C); (7814 11797851 G A); (7815 11798240 T C); (7816 11798373 C T); (7817 11798500 C A); (7818
11798520 T A); (7819 11798568 A G); (7820 11798618 C T); (7821 11798840 A T); (7822 11798862 G
T); (7823 11798868 T G); (7824 11798869 T A); (7825 11798870 T A); (7826 11798871 T A); (7827
11798888 G A); (7828 11798996 G A); (7829 11799038 C T); (7830 11799168 T C); (7831 11799175 T
C); (7832 11799370 T C); (7833 11799391 G A); (7834 11799415 C G); (7835 11799478 G T); (7836
11800513 A G); (7837 11801071 G T); (7838 11801352 T C); (7839 11801372 GA G); (7840 11801388 C
A); (7841 11801950 G C); (7842 11801977 G T); (7843 11802119 G A); (7844 11802621 T A); (7845
11802677 A AAAAG); (7846 11802739 C T); (7847 11802778 C T); (7848 11802785 T C); (7849 11803082
C T); (7850 11803098 A G); (7851 11803155 A G); (7852 11803223 A G); (7853 11803226 G A); (7854
11803254 C A); (7855 11803404 G A); (7856 11803433 G A); (7857 11803528 A G); (7858 11803875 G
GA); (7859 11804033 A G); (7860 11804136 A G); (7861 11804261 G C); (7862 11804427 G C); (7863
11804556 T C); (7864 11804579 G C); (7865 11804717 T G); (7866 11804870 A C); (7867 11805270 C
T); (7868 11805392 A T); (7869 11805722 AC A); (7870 11805932 G A); (7871 11806624 A G); (7872
11807555 C T); (7873 11808370 G A); (7874 11808447 C T); (7875 11808454 C T); (7876 11808825 G
A); (7877 11809176 G T); (7878 11809329 T C); (7879 11809537 C G); (7880 11810140 C T); (7881
11810153 A G); (7882 11811005 A C); (7883 11811822 C T); (7884 11812121 T C); (7885 11812495 A
G); (7886 11812727 T A); (7887 11812991 C A); (7888 11813141 A T); (7889 11813241 T TC); (7890
11813424 T C); (7891 11813443 T A); (7892 11813462 G GT); (7893 11813467 C A); (7894 11813553 A
G); (7895 11813771 A G); (7896 11813784 T C); (7897 11813997 C T); (7898 11814626 G A); (7899
11814735 C A); (7900 11814738 T C); (7901 11814897 A T); (7902 11814922 A C); (7903 11815084 T
G); (7904 11815117 A G); (7905 11815169 G A); (7906 11815898 A G); (7907 11816074 A G); (7908
11816169 A C); (7909 11816285 GA G); (7910 11817229 C A); (7911 11817249 T C); (7912 11817335 T
C); (7913 11817366 G A); (7914 11817719 A G); (7915 11817887 G T); (7916 11819172 T G); (7917
11828624 C T); (7918 11829356 A T); (7919 11830416 C A); (7920 11830474 T C); (7921 11830477 C
T); (7922 11831336 T C); (7923 11831709 C T); (7924 11831884 T C); (7925 11832014 C T); (7926
11832149 G T); (7927 11832275 T A); (7928 11832337 T A); (7929 11832533 C T); (7930 11832573 A
T); (7931 11832936 T C); (7932 11833143 A G); (7933 11833396 A G); (7934 11833585 A G); (7935
11833686 A G); (7936 11833690 T C); (7937 11833777 G A); (7938 11833837 C T); (7939 11834148 G
A); (7940 11834190 G T); (7941 11834236 C T); (7942 11834403 A C); (7943 11835085 A G); (7944
11835102 A G); (7945 11835284 G A); (7946 11835740 A G); (7947 11835830 T C); (7948 11835905 C
T); (7949 11836029 T C); (7950 11836043 T C); (7951 11836305 G C); (7952 11836718 C A); (7953
11836764 T A); (7954 11837205 G T); (7955 11837244 A G); (7956 11837600 A G); (7957 11837736 C
A); (7958 11837747 G A); (7959 11837991 A G); (7960 11838110 A C); (7961 11838937 A G); (7962
11839340 C A); (7963 11839566 C T); (7964 11839643 T G); (7965 11839875 C T); (7966 11839995 A
C); (7967 11840013 A AAGCT); (7968 11840100 A G); (7969 11840249 A G); (7970 11840467 T C); (7971
11840530 G T); (7972 11840775 A AT); (7973 11841329 C T); (7974 11841666 A G); (7975 11841957 G
A); (7976 11842003 A G); (7977 11842136 A C); (7978 11842312 A G); (7979 11842485 T C); (7980
11842545 T C); (7981 11842898 G A); (7982 11842947 GA G); (7983 11842987 G A); (7984 11843767 A
G); (7985 11844273 C T); (7986 11844385 C A); (7987 11844415 C CTAAT); (7988 11844772 A G); (7989
11844952 T A); (7990 11845048 GA G); (7991 11845286 T C); (7992 11845334 T C); (7993 11845344 C
T); (7994 11846541 A AT); (7995 11846732 C A); (7996 11847102 G A); (7997 11847680 C T); (7998
11847749 T C); (7999 11847881 G A); (8000 11848180 T C); (8001 11848218 A G); (8002 11848242 C
A); (8003 11848259 G A); (8004 11848307 T G); (8005 11848531 T C); (8006 11848536 C T); (8007
11848913 A C); (8008 11849353 A G); (8009 11849554 A G); (8010 11850862 G T); (8011 11851195 G
C); (8012 11851294 C T); (8013 11851295 A T); (8014 11851985 A G); (8015 11852032 T C); (8016
11852154 A G); (8017 11852224 T C); (8018 11852484 T C); (8019 11852775 G A); (8020 11853115 T
C); (8021 11853144 G A); (8022 11853257 T C); (8023 11853305 G A); (8024 11853325 G A); (8025
11853361 A T); (8026 11853363 AC A); (8027 11853526 T C); (8028 11853577 C T); (8029 11853586 T
C); (8030 11853848 C T); (8031 11853982 T G); (8032 11854226 T C); (8033 11854417 G A); (8034
11854420 C T); (8035 11854486 A G); (8036 11854583 C T); (8037 11854847 G A); (8038 11855059 A
G); (8039 11855232 G A); (8040 11855245 T C); (8041 11855272 G A); (8042 11856254 A G); (8043
11856378 C T); (8044 11856450 G T); (8045 11857258 A T); (8046 11857571 T C); (8047 11859130 A
G); (8048 11859402 T G); (8049 11860089 G A); (8050 11860164 T C); (8051 11860174 G A); (8052
11860349 TGG T); (8053 11860705 A C); (8054 11860736 C G); (8055 11860769 T C); (8056 11860789 C
CGCGCTGATCGCGCCACTCTG); (8057 11860812 T G); (8058 11861070 A G); (8059 11861351 T C); (8060
11861460 G A); (8061 11861751 G A); (8062 11861845 T C); (8063 11862107 G A); (8064 11862128 C
A); (8065 11862262 A G); (8066 11862362 C A); (8067 11862407 T C); (8068 11862572 T G); (8069
11863216 G A); (8070 11863499 T A); (8071 11863811 A G); (8072 11864307 C T); (8073 11864403 G
A); (8074 11864572 C T); (8075 11864609 G T); (8076 11864691 G A); (8077 11865580 C A); (8078
11866012 C A); (8079 11866065 T G); (8080 11866135 T C); (8081 11866291 G A); (8082 11866666 T
C); (8083 11866816 G A); (8084 11866824 G A); (8085 11866852 G A); (8086 11867120 A C); (8087
11867248 T C); (8088 11867412 G A); (8089 11867502 C A); (8090 11867603 C T); (8091 11867624 AC
A); (8092 11867739 G A); (8093 11867805 A G); (8094 11867827 C T); (8095 11868197 C T); (8096
11868451 G T); (8097 11868474 C G); (8098 11868551 AATAT A); (8099 11868927 T C); (8100 11869136
C T); (8101 11869843 C T); (8102 11870135 T G); (8103 11870201 C A); (8104 11870422 G A); (8105
11870635 A G); (8106 11870925 G A); (8107 11871035 T C); (8108 11871072 G A); (8109 11871079 G
A); (8110 11871126 T A); (8111 11871127 T G); (8112 11871212 C T); (8113 11871303 A G); (8114
11871331 T C); (8115 11871432 A G); (8116 11871441 T C); (8117 11871585 T A); (8118 11871616 A
C); (8119 11871725 G A); (8120 11871742 G GC); (8121 11871975 T G); (8122 11872179 C T); (8123
11872340 C T); (8124 11872382 C T); (8125 11872903 G A); (8126 11872979 C T); (8127 11873003 C
T); (8128 11873083 G A); (8129 11873156 C T); (8130 11873334 C T); (8131 11873452 C T); (8132
11873523 T C); (8133 11873804 G A); (8134 11873965 C T); (8135 11874013 A G); (8136 11874116 C
T); (8137 11874134 A G); (8138 11874211 C G); (8139 11874397 A G); (8140 11874583 T C); (8141
11874690 T C); (8142 11874830 T C); (8143 11874854 G A); (8144 11875005 G A); (8145 11875051 T
C); (8146 11875115 C T); (8147 11875127 G A); (8148 11875559 T C); (8149 11875696 A T); (8150
11875917 G T); (8151 11875945 T C); (8152 11876256 TG T); (8153 11876391 C T); (8154 11876531 G
A); (8155 11876797 G A); (8156 11877172 G A); (8157 11877521 A G); (8158 11877744 G A); (8159
11877811 G T); (8160 11877834 G C); (8161 11877866 A AC); (8162 11877896 T G); (8163 11878753 G
A); (8164 11878886 G A); (8165 11878925 A G); (8166 11878969 C T); (8167 11879247 G A); (8168
11879359 C T); (8169 11880142 A G); (8170 11880219 T C); (8171 11880466 T C); (8172 11880483 A
G); (8173 11880508 C T); (8174 11880931 A C); (8175 11880939 A G); (8176 11881320 A G); (8177
11881327 C T); (8178 11881381 A G); (8179 11882013 C T); (8180 11882076 T A); (8181 11882374 G
A); (8182 11882419 C T); (8183 11882483 C T); (8184 11882551 T C); (8185 11882560 C T); (8186
11882796 G A); (8187 11882826 G A); (8188 11882858 G T); (8189 11883023 C G); (8190 11883169 C
T); (8191 11883255 G A); (8192 11883302 G A); (8193 11883363 T C); (8194 11883424 A G); (8195
11883463 C A); (8196 11883815 T A); (8197 11884061 T C); (8198 11884212 A T); (8199 11886054 A
G); (8200 11886414 C T); (8201 11886912 G A); (8202 11886917 A G); (8203 11887481 G A); (8204
11888802 T C); (8205 11888847 C T); (8206 11888938 G C); (8207 11888988 A G); (8208 11893700 G
A); (8209 11896362 TA T); (8210 11896471 C T); (8211 11896557 G A); (8212 11896702 C T); (8213
11897448 T C); (8214 11897485 G A); (8215 11897516 A T); (8216 11897711 A G); (8217 11897762 A
G); (8218 11897855 T C); (8219 11898042 G A); (8220 11898383 A G); (8221 11899655 G T); (8222
11899667 T C); (8223 11899785 A G); (8224 11899839 G A); (8225 11901240 G A); (8226 11901260 T
C); (8227 11901287 C T); (8228 11901310 G A); (8229 11901747 C T); (8230 11901998 T A); (8231
11903175 C T); (8232 11906552 C T); (8233 11907722 C A); (8234 11909736 T C); (8235 11909824 A
G); (8236 11909946 A G); (8237 11912222 G T); (8238 11912253 A G); (8239 11912281 A G); (8240
11912297 C T); (8241 11912634 A G); (8242 11912648 T C); (8243 11913133 A G); (8244 11913244 A
G); (8245 11913262 C G); (8246 11913309 A G); (8247 11913696 T C); (8248 11914556 T C); (8249
11914565 C T); (8250 11914633 A G); (8251 11914760 T C); (8252 11914845 T C); (8253 11915411 G
A); (8254 11915533 T TACCTAAC); (8255 11915886 G A); (8256 11915899 CTGATGGCTGGG C); (8257
11916055 G A); (8258 11916271 T C); (8259 11916987 A C); (8260 11917060 G T); (8261 11917101 T
C); (8262 11917204 G A); (8263 11917477 G A); (8264 11917538 G A); (8265 11917554 A G); (8266
11917793 C T); (8267 11917805 C A); (8268 11918485 CT C); (8269 11919438 T C); (8270 11919466 T
C); (8271 11919798 G A); (8272 11920960 T A); (8273 11921511 T C); (8274 11921907 A T); (8275
11922304 G A); (8276 11922414 C T); (8277 11923678 T A); (8278 11923717 G T); (8279 11923857 T
TCATGAA); (8280 11923860 A G); (8281 11924040 T C); (8282 11924052 G T); (8283 11924126 A
G); (8284 11924250 C A); (8285 11924723 G A); (8286 11925747 C T); (8287 11926028 T C); (8288
11926396 C T); (8289 11926493 A C); (8290 11927399 A G); (8291 11927432 C T); (8292 11927433 G
A); (8293 11927509 A G); (8294 11927625 A G); (8295 11927870 C T); (8296 11929664 C T); (8297
11930623 C T); (8298 11930739 G A); (8299 11930759 C T); (8300 11930931 C T); (8301 11931014 A
G); (8302 11931268 A C); (8303 11931493 G A); (8304 11931834 T C); (8305 11931899 G A); (8306
11932509 A C); (8307 11932625 T G); (8308 11932797 G T); (8309 11932925 A T); (8310 11933011 G
T); (8311 11933258 T A); (8312 11933386 T G); (8313 11933424 C T); (8314 11933607 G A); (8315
11933670 T TAGATCA); (8316 11934476 G A); (8317 11934658 A T); (8318 11934660 A AAGTT); (8319
11935231 G A); (8320 11935492 C A); (8321 11935979 T C); (8322 11936170 T C); (8323 11936187 C
G); (8324 11936651 G A); (8325 11936706 T G); (8326 11936804 C CAT); (8327 11937439 C T); (8328
11939964 T C); (8329 11940210 A G); (8330 11940277 A G); (8331 11940296 G A); (8332 11940399 C
T); (8333 11940458 T C); (8334 11940570 A G); (8335 11940974 A G); (8336 11941183 C A); (8337
11941356 C T); (8338 11941434 C T); (8339 11941642 T C); (8340 11941668 A G); (8341 11941732 A
G); (8342 11941735 G A); (8343 11941877 A G); (8344 11942059 T C); (8345 11942287 G A); (8346
11942543 G A); (8347 11945426 C A); (8348 11945631 G A); (8349 11946303 G A); (8350 11946367 A
G); (8351 11946495 T C); (8352 11946503 G A); (8353 11946642 C T); (8354 11946806 T C); (8355
11947091 A T); (8356 11947176 A G); (8357 11948074 T C); (8358 11948363 T A); (8359 11948381 C
T); (8360 11948789 T C); (8361 11948874 T A); (8362 11949204 T C); (8363 11949244 G A); (8364
11950154 C T); (8365 11950251 A C); (8366 11951537 G A); (8367 11952257 G A); (8368 11953463 G
C); (8369 11953584 G A); (8370 11953587 G A); (8371 11961555 ATGGTGAATG A); (8372 11962179 G
A); (8373 11962377 C T); (8374 11962528 C T); (8375 11963381 T C); (8376 11964130 G T); (8377
11964407 C T); (8378 11964599 A G); (8379 11965148 A T); (8380 11965946 T TA); (8381 11965988 A
G); (8382 11966024 G A); (8383 11966132 G A); (8384 11966226 CA C); (8385 11966322 C T); (8386
11966672 T C); (8387 11967230 A G); (8388 11967422 A G); (8389 11967488 G A); (8390 11968602 C
A); (8391 11970752 G A); (8392 11971063 G A); (8393 11971342 AT A); (8394 11971346 G A); (8395
11971669 G A); (8396 11972101 A T); (8397 11972706 G A); (8398 11973073 A G); (8399 11973167 A
G); (8400 11973241 A G); (8401 11973279 A G); (8402 11973302 C T); (8403 11973583 G A); (8404
11973658 T C); (8405 11973687 T C); (8406 11973896 G A); (8407 11973971 T G); (8408 11974000 A
G); (8409 11974092 T C); (8410 11974145 G T); (8411 11974587 G A); (8412 11974889 C T); (8413
11975208 T TAAGAGCTAAAAG); (8414 11975284 G A); (8415 11975310 C A); (8416 11975363 A
G); (8417 11975394 C T); (8418 11975550 A G); (8419 11975582 C T); (8420 11976098 T G); (8421
11976186 C T); (8422 11976449 T C); (8423 11976512 C T); (8424 11977174 G T); (8425 11977177 A
G); (8426 11977268 T G); (8427 11977376 T C); (8428 11977386 T C); (8429 11978011 T C); (8430
11978663 G A); (8431 11978804 C T); (8432 11980153 G A); (8433 11980294 T C); (8434 11980882 T
C); (8435 11981060 T G); (8436 11981133 A G); (8437 11981410 C T); (8438 11982167 G A); (8439
11982243 G A); (8440 11982285 A G); (8441 11982554 C T); (8442 11982602 T C); (8443 11984036 T
C); (8444 11985836 T C); (8445 11985892 T C); (8446 11985945 A G); (8447 11986297 C T); (8448
11986524 C T); (8449 11986915 T G); (8450 11987215 T C); (8451 11988223 A G); (8452 11988570 C
T); (8453 11989064 T C); (8454 11989082 C T); (8455 11989270 CAT C); (8456 11989566 C G); (8457
11989742 A G); (8458 11989813 G A); (8459 11990036 C T); (8460 11990332 A G); (8461 11990357 G
A); (8462 11991528 A G); (8463 11991783 A C); (8464 11991819 C T); (8465 11992059 T C); (8466
11992076 G A); (8467 11992795 A G); (8468 11992995 C T); (8469 11993157 G C); (8470 11993346 T
C); (8471 11993478 T C); (8472 11993750 T TA); (8473 11994091 G C); (8474 11994516 C G); (8475
11994551 A T); (8476 11994733 A AC); (8477 11994912 C T); (8478 11994939 A C); (8479 11995213 C
A); (8480 11995624 G C); (8481 11995808 A T); (8482 11995935 A C); (8483 11996417 A C); (8484
11997166 A G); (8485 11997242 A G); (8486 11997400 A G); (8487 11997472 G A); (8488 12001407 C
T); (8489 12002849 T C); (8490 12003154 C T); (8491 12003406 T C); (8492 12003673 T C); (8493
12003694 T C); (8494 12003731 C T); (8495 12003771 C T); (8496 12003912 C T); (8497 12003916 C
T); (8498 12004943 C T); (8499 12005630 G A); (8500 12006245 T G); (8501 12006246 T G); (8502
12006664 T G); (8503 12007044 T A); (8504 12007058 A AC); (8505 12007132 C A); (8506 12007181 G
A); (8507 12007292 A G); (8508 12007299 T C); (8509 12007396 G A); (8510 12007838 A G); (8511
12007901 A G); (8512 12008076 C T); (8513 12011032 G A); (8514 12011445 G A); (8515 12011461 G
T); (8516 12011579 C T); (8517 12011797 A G); (8518 12012163 C G); (8519 12012305 C A); (8520
12012360 CATTT C); (8521 12012800 A T); (8522 12012999 T C); (8523 12013016 A G); (8524 12013348
C A); (8525 12013372 C T); (8526 12013931 A C); (8527 12016363 G GT); (8528 12016422 A G); (8529
12016653 T C); (8530 12016675 T C); (8531 12016739 G A); (8532 12016778 T C); (8533 12017339 C
T); (8534 12017559 G A); (8535 12017766 C T); (8536 12018156 A C); (8537 12018853 A T); (8538
12018988 C T); (8539 12019008 A G); (8540 12019796 A G); (8541 12020084 T C); (8542 12020456 C
T); (8543 12021053 A G); (8544 12022866 C T); (8545 12022955 CT C); (8546 12023081 C T); (8547
12023607 A G); (8548 12024211 T G); (8549 12025399 T C); (8550 12025433 TCTAG T); (8551 12026608
T C); (8552 12026804 C A); (8553 12027468 G A); (8554 12027601 G A); (8555 12027634 T C); (8556
12028252 G A); (8557 12028540 G A); (8558 12028623 T C); (8559 12028678 G C); (8560 12028850 T
C); (8561 12029056 C T); (8562 12029164 C CAACG); (8563 12029711 A G); (8564 12029801 G A); (8565
12030440 AC A); (8566 12030804 TA T); (8567 12031388 C A); (8568 12031407
ACGTTTATATTTAAGCAAT A); (8569 12032214 T C); (8570 12032929 C T); (8571 12033379
GTATTTAAACCTA G); (8572 12033604 T A); (8573 12033605 A T); (8574 12033606 T A); (8575 12035267
A G); (8576 12035312 G A); (8577 12035339 C T); (8578 12035648 A T); (8579 12035688 G A); (8580
12035730 A G); (8581 12036234 A G); (8582 12036373 C T); (8583 12036419 T C); (8584 12036656 G
A); (8585 12036688 G A); (8586 12036765 G A); (8587 12036783 C T); (8588 12036877 A G); (8589
12037046 A C); (8590 12037124 T A); (8591 12037137 C T); (8592 12037191 T C); (8593 12037320 C
T); (8594 12037434 G T); (8595 12037486 T C); (8596 12037491 C T); (8597 12038107 T C); (8598
12038354 T C); (8599 12038362 C T); (8600 12038436 T C); (8601 12038451 G A); (8602 12038602 C
A); (8603 12038783 T C); (8604 12039048 C T); (8605 12039174 C T); (8606 12039384 C G); (8607
12039393 C T); (8608 12041953 A G); (8609 12042040 G A); (8610 12042100 T G); (8611 12042144 G
A); (8612 12042241 A G); (8613 12042331 G C); (8614 12042598 G A); (8615 12042933 C T); (8616
12043207 A C); (8617 12043226 T A); (8618 12043318 C T); (8619 12043350 G T); (8620 12044065 A
G); (8621 12044081 T C); (8622 12044166 A G); (8623 12044172 T G); (8624 12044284 A G); (8625
12044342 T C); (8626 12044407 G A); (8627 12044414 G A); (8628 12044420 G A); (8629 12044426 G
T); (8630 12044459 G A); (8631 12044570 T A); (8632 12044652 C T); (8633 12044843 T C); (8634
12044953 T C); (8635 12045072 T C); (8636 12045119 G A); (8637 12045121 T C); (8638 12045144 A
C); (8639 12045352 G A); (8640 12045371 G A); (8641 12045396 G A); (8642 12045417 T C); (8643
12045422 C T); (8644 12045547 C T); (8645 12045569 T A); (8646 12045579 T C); (8647 12045662 G
A); (8648 12045837 C T); (8649 12045977 T C); (8650 12046258 A G); (8651 12046576 G C); (8652
12046607 G A); (8653 12046631 T G); (8654 12046785 G A); (8655 12047097 G A); (8656 12047585 A
G); (8657 12047604 T C); (8658 12047859 G A); (8659 12048742 T TA); (8660 12048824 A G); (8661
12049419 TTA T); (8662 12049521 G T); (8663 12050196 G GA); (8664 12050518 AT A); (8665 12050679
A AT); (8666 12051195 TTA T); (8667 12051318 A G); (8668 12051559 C T); (8669 12053450 G T); (8670
12053789 G T); (8671 12054868 AT A); (8672 12054928 T TA); (8673 12055225 A T); (8674 12055343 C
G); (8675 12055891 C T); (8676 12056009 C T); (8677 12056036 G A); (8678 12056079 A G); (8679
12056269 A T); (8680 12056366 G T); (8681 12056925 G T); (8682 12056993 G GATA); (8683 12057035
G A); (8684 12058032 TA T); (8685 12058417 A G); (8686 12058444 T C); (8687 12059127 G A); (8688
12059158 A G); (8689 12059300 A G); (8690 12059455 G T); (8691 12059533 C T); (8692 12059570 A
G); (8693 12059601 C A); (8694 12059867 G A); (8695 12059875 G GC); (8696 12059936 G A); (8697
12060110 T C); (8698 12060112 G C); (8699 12060140 C G); (8700 12060357 T C); (8701 12060364 A
G); (8702 12060607 AC A); (8703 12060950 C A); (8704 12061489 T C); (8705 12063597 A G); (8706
12063634 C T); (8707 12064079 T C); (8708 12064086 A G); (8709 12064613 G A); (8710 12065132 TA
T); (8711 12065338 A G); (8712 12065694 G A); (8713 12065773 T C); (8714 12066781 C T); (8715
12066796 C T); (8716 12066863 C T); (8717 12066976 T A); (8718 12067038 C T); (8719 12067173 T
G); (8720 12067301 T C); (8721 12067303 C T); (8722 12067401 G C); (8723 12067501 TA T); (8724
12067615 T C); (8725 12067782 T C); (8726 12067871 C G); (8727 12067953 G A); (8728 12068040 T
G); (8729 12068231 C T); (8730 12069340 A G); (8731 12069464 G T); (8732 12069496 C T); (8733
12070525 T C); (8734 12070967 C G); (8735 12071263 T C); (8736 12071521 C T); (8737 12071552 A
G); (8738 12071589 G A); (8739 12071620 C T); (8740 12071632 A G); (8741 12071962 C T); (8742
12080278 T A); (8743 12080325 G A); (8744 12081097 T C); (8745 12081357 T A); (8746 12081581 C
T); (8747 12081615 C T); (8748 12081657 A C); (8749 12081766 C T); (8750 12081911 A G); (8751
12082099 CAAT C); (8752 12082292 G C); (8753 12082311 A G); (8754 12082902 C T); (8755 12083078 A
G); (8756 12083668 G A); (8757 12083721 AT A); (8758 12083789 A G); (8759 12084283 G A); (8760
12084420 G C); (8761 12085135 C T); (8762 12085321 A G); (8763 12086107 G A); (8764 12087132 A
G); (8765 12087217 G A); (8766 12087534 G A); (8767 12087565 T C); (8768 12088522 G A); (8769
12089202 T C); (8770 12089418 A T); (8771 12090443 G A); (8772 12090528 C T); (8773 12091343 A
G); (8774 12091361 A G); (8775 12091651 A G); (8776 12091713 T C); (8777 12092787 C A); (8778
12092997 T C); (8779 12093153 A G); (8780 12093216 A C); (8781 12094036 G A); (8782 12094064 T
C); (8783 12094784 G A); (8784 12095232 G A); (8785 12095541 G T); (8786 12095672 G C); (8787
12096397 G A); (8788 12096645 T C); (8789 12096680 G A); (8790 12097015 T G); (8791 12097584 A
C); (8792 12097672 G A); (8793 12097711 A G); (8794 12097806 C T); (8795 12097928 C A); (8796
12098114 T C); (8797 12098222 G C); (8798 12098411 T C); (8799 12099023 G A); (8800 12100044 G
A); (8801 12100114 C A); (8802 12100286 C T); (8803 12100392 A C); (8804 12100400 A G); (8805
12101080 T C); (8806 12101370 C T); (8807 12101679 G A); (8808 12101901 C T); (8809 12102074 C
A); (8810 12102374 T C); (8811 12102869 T C); (8812 12103807 A G); (8813 12103865 G A); (8814
12103880 G T); (8815 12104354 C T); (8816 12104677 A G); (8817 12105017 A T); (8818 12105954 A
G); (8819 12106088 A G); (8820 12106178 C A); (8821 12106780 C T); (8822 12106983 T A); (8823
12107175 A G); (8824 12108360 G A); (8825 12108881 T A); (8826 12109062 T C); (8827 12109067 T
C); (8828 12109465 C T); (8829 12109537 T A); (8830 12109929 G A); (8831 12110392 C A); (8832
12110439 A G); (8833 12110719 A C); (8834 12111970 T C); (8835 12112289 A G); (8836 12113179 A
G); (8837 12113206 A G); (8838 12113393 A T); (8839 12113588 C CT); (8840 12114282 A T); (8841
12116743 G A); (8842 12118526 T C); (8843 12118531 A G); (8844 12118733 C T); (8845 12119499 A
G); (8846 12119800 G A); (8847 12119868 C T); (8848 12119967 T G); (8849 12119987 T C); (8850
12120012 A G); (8851 12120409 A G); (8852 12120785 A G); (8853 12120878 G C); (8854 12120906 A
G); (8855 12120923 G A); (8856 12121158 A G); (8857 12121218 G A); (8858 12121735 A G); (8859
12121741 T C); (8860 12122410 T C); (8861 12123030 T C); (8862 12123071 A G); (8863 12123102 C
T); (8864 12126291 C T); (8865 12126306 A G); (8866 12126346 A G); (8867 12126416 G C); (8868
12126422 A G); (8869 12126758 G A); (8870 12127190 A C); (8871 12127234 A C); (8872 12127284 C
T); (8873 12127979 A T); (8874 12128027 G A); (8875 12128341 G A); (8876 12128353 G C); (8877
12128387 C A); (8878 12128474 C T); (8879 12128761 T C); (8880 12129090 T A); (8881 12129175 A
G); (8882 12129416 C T); (8883 12129425 A G); (8884 12129495 A AT); (8885 12129556 T A); (8886
12129934 CCG C); (8887 12129988 A C); (8888 12130496 A G); (8889 12130687 CT C); (8890 12131744 C
CAT); (8891 12132535 C CTTTAA); (8892 12133447 A G); (8893 12133471 T C); (8894 12133550 A
T); (8895 12133875 A G); (8896 12134158 C A); (8897 12134247 A AAT); (8898 12134307 A AC); (8899
12134846 C CT); (8900 12135505 A T); (8901 12135744 G T); (8902 12135948 G A); (8903 12136109 G
A); (8904 12136168 C G); (8905 12136859 A G); (8906 12137143 C T); (8907 12137300 G A); (8908
12137506 T C); (8909 12137517 G C); (8910 12137531 C T); (8911 12137611 G A); (8912 12137878 T
C); (8913 12138518 T C); (8914 12138671 C A); (8915 12139319 GA G); (8916 12139456 C T); (8917
12139599 C T); (8918 12139735 G A); (8919 12139850 A G); (8920 12140749 T G); (8921 12141075 CTTA
C); (8922 12142159 A G); (8923 12142570 T C); (8924 12142585 A T); (8925 12142618 C A); (8926
12142632 T C); (8927 12143169 C T); (8928 12143600 A G); (8929 12143616 T A); (8930 12144009 G
A); (8931 12144042 T C); (8932 12144669 C A); (8933 12144882 GA G); (8934 12145116 A AT); (8935
12145362 G A); (8936 12145794 G A); (8937 12146383 AAG A); (8938 12146601 T G); (8939 12146893 A
G); (8940 12146916 T A); (8941 12146959 G GA); (8942 12147059 CT C); (8943 12147082 T A); (8944
12147221 A G); (8945 12147352 A T); (8946 12147443 T C); (8947 12147621 A G); (8948 12147894 A
G); (8949 12148458 C T); (8950 12148887 C T); (8951 12148919 CA C); (8952 12149121 T C); (8953
12149189 G A); (8954 12149254 T G); (8955 12149330 C T); (8956 12149468 T C); (8957 12150314 A
C); (8958 12150744 A G); (8959 12150807 T G); (8960 12150846 A G); (8961 12150959 G A); (8962
12151528 G A); (8963 12151599 G A); (8964 12151612 G T); (8965 12152147 G T); (8966 12152341 A
G); (8967 12152440 G A); (8968 12152494 G T); (8969 12152530 T C); (8970 12153655 T C); (8971
12153678 G A); (8972 12153732 A G); (8973 12153862 ATACT A); (8974 12154163 A C); (8975 12154512
G A); (8976 12155104 A G); (8977 12155130 C T); (8978 12155597 A G); (8979 12156114 G T); (8980
12156223 T G); (8981 12156861 T C); (8982 12156912 G A); (8983 12157433 T C); (8984 12157640 AT
A); (8985 12157835 G A); (8986 12158060 T C); (8987 12158082 T C); (8988 12158093 C T); (8989
12158140 A G); (8990 12158154 T G); (8991 12158192 A G); (8992 12158198 C T); (8993 12158202 A
G); (8994 12158215 C T); (8995 12158595 T C); (8996 12159739 TA T); (8997 12159804 A
AATATAT); (8998 12161864 A AT); (8999 12162913 GA G); (9000 12162988 A T); (9001 12163589 A
G); (9002 12163843 GA G); (9003 12163887 T C); (9004 12163943 G A); (9005 12163966 C T); (9006
12163970 C T); (9007 12164355 A AATATAT); (9008 12164458 T A); (9009 12164461 A G); (9010
12164462 G T); (9011 12164463 T A); (9012 12164574 TA T); (9013 12164894 C A); (9014 12165774 T
C); (9015 12166007 T C); (9016 12166114 G A); (9017 12166119 G A); (9018 12166133 AC A); (9019
12166379 A G); (9020 12166728 A T); (9021 12166915 A G); (9022 12167229 G A); (9023 12167294 T
C); (9024 12167383 G A); (9025 12167528 G A); (9026 12167762 C A); (9027 12168698 G A); (9028
12168774 A G); (9029 12169191 T A); (9030 12169490 A G); (9031 12169681 A G); (9032 12169724 C
A); (9033 12169733 T G); (9034 12169754 T C); (9035 12170309 T C); (9036 12170435 T C); (9037
12170744 C T); (9038 12170999 C T); (9039 12171347 C T); (9040 12171351 T G); (9041 12171728 G
A); (9042 12171819 C T); (9043 12172872 G A); (9044 12172957 C T); (9045 12173055 T C); (9046
12173660 G A); (9047 12174319 C T); (9048 12174391 A T); (9049 12175361 C T); (9050 12175805 C
T); (9051 12176237 C G); (9052 12176499 G A); (9053 12176674 A G); (9054 12177037 T C); (9055
12177187 A G); (9056 12177230 T C); (9057 12177293 T C); (9058 12178862 G A); (9059 12180387 C
T); (9060 12183128 T C); (9061 12186783 A G); (9062 12186938 C T); (9063 12186953 C T); (9064
12187024 G A); (9065 12187177 C T); (9066 12187381 T C); (9067 12187394 T C); (9068 12189015 T
A); (9069 12189099 G T); (9070 12189124 T C); (9071 12189286 C A); (9072 12189315 A G); (9073
12189454 C T); (9074 12190042 A G); (9075 12190599 G A); (9076 12190690 A G); (9077 12190735 G
A); (9078 12190910 C T); (9079 12191261 ATTT A); (9080 12191333 A T); (9081 12191521 C T); (9082
12191638 T C); (9083 12192599 A C); (9084 12193271 A C); (9085 12193605 G A); (9086 12193649 C
T); (9087 12195611 T C); (9088 12195708 C T); (9089 12195889 T C); (9090 12196016 C T); (9091
12196415 A G); (9092 12196950 C T); (9093 12197619 T C); (9094 12197776 A G); (9095 12197989 C
T); (9096 12198240 GA G); (9097 12198574 T C); (9098 12198800 T C); (9099 12199084 T C); (9100
12199236 G A); (9101 12199240 T C); (9102 12199316 T G); (9103 12199372 G A); (9104 12200008 G
C); (9105 12200146 A G); (9106 12200307 CTTT C); (9107 12200355 C T); (9108 12200566 A G); (9109
12201512 G A); (9110 12201636 C T); (9111 12201703 C T); (9112 12201734 A G); (9113 12201767 T
C); (9114 12202183 G A); (9115 12202797 G A); (9116 12203541 G A); (9117 12203672 T C); (9118
12203752 G T); (9119 12203849 C T); (9120 12204127 C T); (9121 12205022 T C); (9122 12205031 G
A); (9123 12205495 A G); (9124 12206063 C T); (9125 12206276 A T); (9126 12207053 T TA); (9127
12208241 T A); (9128 12209001 C A); (9129 12209309 A G); (9130 12209669 G GT); (9131 12211274 A
G); (9132 12211303 T C); (9133 12212190 C T); (9134 12213070 C T); (9135 12213557 G A); (9136
12213927 A C); (9137 12214296 G A); (9138 12216228 A G); (9139 12216377 A T); (9140 12217259 T
A); (9141 12218138 G A); (9142 12218167 C T); (9143 12218181 G A); (9144 12218195 A G); (9145
12218205 C G); (9146 12218363 G A); (9147 12218462 G A); (9148 12219170 C T); (9149 12219195 C
T); (9150 12219262 A ATC); (9151 12219291 T G); (9152 12219347 AACTT A); (9153 12219712 T C); (9154
12220074 A G); (9155 12220306 G A); (9156 12220921 G A); (9157 12221143 T G); (9158 12221171 G
A); (9159 12221923 T C); (9160 12221952 G T); (9161 12222602 G T); (9162 12222846 A G); (9163
12223749 G A); (9164 12223811 T C); (9165 12223831 C T); (9166 12224695 G T); (9167 12224995 G
T); (9168 12226848 C T); (9169 12226905 A G); (9170 12226996 C T); (9171 12227145 A G); (9172
12227254 C T); (9173 12227405 T G); (9174 12227504 T G); (9175 12227808 T TA); (9176 12227827 C
T); (9177 12227831 A C); (9178 12227863 A G); (9179 12227912 T C); (9180 12227915 A G); (9181
12227989 A G); (9182 12228629 C G); (9183 12229870 G A); (9184 12230194 C A); (9185 12230694 G
A); (9186 12230934 C T); (9187 12231444 A C); (9188 12232584 A G); (9189 12232709 G A); (9190
12232737 T C); (9191 12232832 C T); (9192 12233789 G A); (9193 12234081 T C); (9194 12234109 A
C); (9195 12234177 T C); (9196 12234199 C A); (9197 12234269 G A); (9198 12236218 T G); (9199
12236239 A G); (9200 12236428 G A); (9201 12237086 A C); (9202 12237764 C A); (9203 12237829 A
G); (9204 12238276 T A); (9205 12238606 G T); (9206 12238658 AT A); (9207 12239248 G A); (9208
12240197 A G); (9209 12241008 G A); (9210 12241282 C T); (9211 12241302 C T); (9212 12242737 G
A); (9213 12243007 C T); (9214 12243022 A G); (9215 12243326 G A); (9216 12243400 G GA); (9217
12243453 T C); (9218 12243464 A G); (9219 12243505 C T); (9220 12245096 G A); (9221 12245987 ATT
A); (9222 12246860 C T); (9223 12247707 A T); (9224 12247732 GGTT G); (9225 12248688 G A); (9226
12249426 A G); (9227 12251399 C T); (9228 12251411 G T); (9229 12252112 A G); (9230 12252677 T
G); (9231 12252818 A T); (9232 12253222 A AT); (9233 12253371 T C); (9234 12253579 C A); (9235
12253611 A C); (9236 12253895 T C); (9237 12254071 T C); (9238 12254146 TG T); (9239 12254150 G
T); (9240 12255015 A C); (9241 12255161 A T); (9242 12255661 T C); (9243 12255755 C CA); (9244
12255862 A G); (9245 12255901 G A); (9246 12255960 A T); (9247 12256452 AT A); (9248 12256567 T
A); (9249 12256886 G A); (9250 12257053 C A); (9251 12257289 G A); (9252 12257295 G A); (9253
12257370 G A); (9254 12257397 G A); (9255 12257415 G A); (9256 12257506 T C); (9257 12258121 G
C); (9258 12258350 A G); (9259 12258351 T C); (9260 12258432 G A); (9261 12258508 C T); (9262
12258582 C T); (9263 12258628 A G); (9264 12258743 A ATT); (9265 12258887 T TA); (9266 12259248 T
C); (9267 12259304 A G); (9268 12259464 C T); (9269 12259476 C T); (9270 12259477 G A); (9271
12259520 A C); (9272 12261446 C T); (9273 12261717 C T); (9274 12261782 G T); (9275 12262232 T
C); (9276 12262619 G A); (9277 12262744 A T); (9278 12262839 C T); (9279 12263008 C T); (9280
12263078 A T); (9281 12263216 T G); (9282 12263424 G A); (9283 12264121 C A); (9284 12264261 T
A); (9285 12264300 G A); (9286 12264858 T G); (9287 12264962 T C); (9288 12265048 C T); (9289
12265800 A G); (9290 12265905 C A); (9291 12266051 C A); (9292 12266055 G A); (9293 12266270 C
T); (9294 12266287 TA T); (9295 12266828 C T); (9296 12267435 C T); (9297 12267694 T C); (9298
12267793 G A); (9299 12267833 T C); (9300 12267910 A G); (9301 12267961 C T); (9302 12267985 G
C); (9303 12268121 G A); (9304 12269313 C T); (9305 12269445 C G); (9306 12269506 C T); (9307
12269638 G A); (9308 12270006 T C); (9309 12270045 C T); (9310 12270080 T C); (9311 12270714 C
T); (9312 12273390 A G); (9313 12273624 G A); (9314 12276687 A G); (9315 12277432 C A); (9316
12277507 C T); (9317 12277569 T C); (9318 12277593 G A); (9319 12277862 T C); (9320 12278139 G
T); (9321 12278185 T C); (9322 12278233 A G); (9323 12278254 T G); (9324 12278290 A G); (9325
12278364 T C); (9326 12278407 C T); (9327 12278490 G A); (9328 12278497 A G); (9329 12278520 C
A); (9330 12278847 C T); (9331 12279103 C CCATAAGTTAT); (9332 12279114 G A); (9333 12279163 C
T); (9334 12279308 G A); (9335 12279343 C T); (9336 12279380 G T); (9337 12279471 G A); (9338
12279508 A G); (9339 12279744 C T); (9340 12279806 T C); (9341 12279979 G C); (9342 12280920 T
G); (9343 12281893 C T); (9344 12281972 T C); (9345 12282008 T C); (9346 12282343 A T); (9347
12283258 G A); (9348 12284592 A C); (9349 12284646 A G); (9350 12285983 T C); (9351 12286102 A
G); (9352 12286240 A G); (9353 12286466 T C); (9354 12286473 G A); (9355 12286486 G A); (9356
12286506 C T); (9357 12286711 C G); (9358 12287506 G A); (9359 12290029 C G); (9360 12290432 C
T); (9361 12290464 G T); (9362 12290494 A T); (9363 12290507 G A); (9364 12290531 G A); (9365
12290698 C T); (9366 12291063 C A); (9367 12291940 A T); (9368 12292010 C T); (9369 12292051 G
A); (9370 12292177 T C); (9371 12292242 A C); (9372 12292255 T C); (9373 12292292 T C); (9374
12292349 T G); (9375 12292600 C T); (9376 12292833 C T); (9377 12292877 G C); (9378 12293174 GCA
G); (9379 12293219 A G); (9380 12293537 T C); (9381 12293603 A G); (9382 12294280 A G); (9383
12294862 C G); (9384 12295353 C T); (9385 12295462 C T); (9386 12295490 T A); (9387 12295680 A
G); (9388 12295827 C A); (9389 12296211 T C); (9390 12296376 A G); (9391 12296708 A C); (9392
12297294 T A); (9393 12297597 A C); (9394 12297642 T C); (9395 12297729 T G); (9396 12297816 C
T); (9397 12298177 G A); (9398 12298558 A G); (9399 12298629 G T); (9400 12298635 G A); (9401
12299011 A G); (9402 12299496 T TTA); (9403 12299605 A T); (9404 12299677 G A); (9405 12300940 T
C); (9406 12301211 G T); (9407 12301300 A C); (9408 12301964 A AAT); (9409 12302055 A G); (9410
12302874 T A); (9411 12303142 C CA); (9412 12303194 G GA); (9413 12303465 G A); (9414 12303575 A
G); (9415 12303970 T G); (9416 12304436 G T); (9417 12304496 CA C); (9418 12305232 G A); (9419
12305265 G A); (9420 12305605 AT A); (9421 12305748 C A); (9422 12306084 C T); (9423 12306641 T
A); (9424 12306916 CTA C); (9425 12307034 C T); (9426 12307649 G A); (9427 12307908 A G); (9428
12308139 G A); (9429 12308266 A G); (9430 12308435 C T); (9431 12308546 A G); (9432 12308571 T
C); (9433 12308591 G A); (9434 12308678 G A); (9435 12308689 G A); (9436 12309234 T C); (9437
12309255 C T); (9438 12309369 A C); (9439 12309411 T C); (9440 12309755 C T); (9441 12309870 G
A); (9442 12309988 A G); (9443 12310024 C T); (9444 12310809 G A); (9445 12310904 G T); (9446
12311012 T G); (9447 12311291 GA G); (9448 12311555 T C); (9449 12311811 T TG); (9450 12312292 T
C); (9451 12312472 G T); (9452 12313468 GA G); (9453 12314890 A G); (9454 12314922 C T); (9455
12315170 A G); (9456 12315655 T C); (9457 12316127 C A); (9458 12316654 A C); (9459 12316926 T
A); (9460 12317046 A G); (9461 12317128 G A); (9462 12317411 T G); (9463 12317580 A ATAAT); (9464
12317814 G A); (9465 12317942 A G); (9466 12318645 A C); (9467 12318657 G A); (9468 12318687 T
A); (9469 12318777 T G); (9470 12319037 C G); (9471 12319417 T C); (9472 12319519 T A); (9473
12319759 G A); (9474 12319879 G A); (9475 12320634 C T); (9476 12320858 A G); (9477 12321083 A
G); (9478 12321352 G A); (9479 12321816 C A); (9480 12322474 A C); (9481 12322609 G
GACACTTTACTTA); (9482 12322922 T C); (9483 12323025 A G); (9484 12323263 G A); (9485 12323351 T
C); (9486 12323701 G A); (9487 12323932 G T); (9488 12324182 G A); (9489 12324375 G A); (9490
12324393 T C); (9491 12324539 T C); (9492 12325034 T G); (9493 12326057 C T); (9494 12327397 A
G); (9495 12327854 A G); (9496 12328591 T G); (9497 12328655 C G); (9498 12328797 G A); (9499
12328800 G A); (9500 12329183 C T); (9501 12329239 C T); (9502 12329266 A G); (9503 12329328 C
T); (9504 12329382 G A); (9505 12329498 C T); (9506 12329861 G A); (9507 12330427 G GGCAA); (9508
12331355 A G); (9509 12332184 C T); (9510 12332726 G A); (9511 12333089 G C); (9512 12333357 G
A); (9513 12334909 G C); (9514 12334910 C A); (9515 12335773 A G); (9516 12335958 C T); (9517
12336160 G A); (9518 12336555 T C); (9519 12337276 T C); (9520 12337376 G A); (9521 12337699 G
A); (9522 12337744 G T); (9523 12337748 G T); (9524 12337957 T C); (9525 12338006 T G); (9526
12338128 C T); (9527 12339818 A G); (9528 12345542 A C); (9529 12347296 T C); (9530 12348369 T
G); (9531 12348603 A G); (9532 12350092 T C); (9533 12350919 A G); (9534 12350933 G A); (9535
12351044 A G); (9536 12351047 A G); (9537 12351126 A G); (9538 12352027 G A); (9539 12353411 T
C); (9540 12354258 A G); (9541 12354298 C T); (9542 12354365 C T); (9543 12354400 G A); (9544
12354478 A G); (9545 12354560 C T); (9546 12354834 C T); (9547 12354849 T C); (9548 12354941 C
G); (9549 12355850 T C); (9550 12356205 C T); (9551 12356334 G A); (9552 12357230 C T); (9553
12357238 C T); (9554 12357254 A G); (9555 12357554 A G); (9556 12357595 C A); (9557 12357630 G
A); (9558 12357901 G A); (9559 12358013 T C); (9560 12358032 A G); (9561 12358938 A C); (9562
12358945 C T); (9563 12359013 GAT G); (9564 12360702 C T); (9565 12362262 C T); (9566 12362452 GA
G); (9567 12362470 AT A); (9568 12362525 T C); (9569 12362989 C A); (9570 12363456 C T); (9571
12363828 T C); (9572 12363989 G A); (9573 12366662 G A); (9574 12367263 A G); (9575 12367491 T
C); (9576 12367914 C T); (9577 12367919 A C); (9578 12368089 T C); (9579 12368265 C A); (9580
12368919 C T); (9581 12368927 G A); (9582 12369026 C T); (9583 12369080 G C); (9584 12369141 G
A); (9585 12369163 G T); (9586 12369232 G A); (9587 12369233 G A); (9588 12369505 T C); (9589
12369541 T C); (9590 12369754 A G); (9591 12369760 T C); (9592 12370895 C T); (9593 12370906 C
T); (9594 12371180 T G); (9595 12371189 T C); (9596 12371538 C T); (9597 12371722 C T); (9598
12371985 C T); (9599 12372310 GA G); (9600 12372596 G A); (9601 12372742 G T); (9602 12373236 A
G); (9603 12373605 G T); (9604 12373744 G A); (9605 12373753 G T); (9606 12373774 G A); (9607
12373873 G A); (9608 12373903 A C); (9609 12374060 C T); (9610 12374268 C T); (9611 12374528 T
A); (9612 12375388 A G); (9613 12375501 A G); (9614 12375858 C T); (9615 12375958 T C); (9616
12376683 C T); (9617 12377128 T C); (9618 12377228 G GA); (9619 12378279 A G); (9620 12378874 A
G); (9621 12379241 A G); (9622 12379358 C G); (9623 12379380 T C); (9624 12380906 G T); (9625
12381031 G A); (9626 12381157 C G); (9627 12381203 A G); (9628 12381205 T G); (9629 12381372 C
T); (9630 12381454 C T); (9631 12381701 A G); (9632 12381900 G T); (9633 12382101 C T); (9634
12382204 C T); (9635 12382263 C T); (9636 12382417 G A); (9637 12382456 C T); (9638 12382573 G
A); (9639 12382610 T A); (9640 12382776 G A); (9641 12382778 A G); (9642 12382846 A G); (9643
12383194 G A); (9644 12383195 G A); (9645 12383196 G A); (9646 12383211 A G); (9647 12383371 G
C); (9648 12383477 G A); (9649 12383489 T C); (9650 12383504 A G); (9651 12383685 T A); (9652
12383869 A G); (9653 12384061 G A); (9654 12384183 C T); (9655 12384234 A C); (9656 12384245 C
T); (9657 12384434 G A); (9658 12384594 A T); (9659 12385085 A C); (9660 12385123 C T); (9661
12385255 G A); (9662 12385257 T C); (9663 12385338 G A); (9664 12385340 T C); (9665 12385482 A
C); (9666 12386153 A C); (9667 12386206 G T); (9668 12386322 C A); (9669 12386593 C A); (9670
12386719 A G); (9671 12386851 T G); (9672 12387148 A G); (9673 12387167 C T); (9674 12387297 G
A); (9675 12387649 A C); (9676 12387655 A G); (9677 12388001 G A); (9678 12388050 G A); (9679
12388063 G A); (9680 12388130 A G); (9681 12388537 G A); (9682 12388691 T G); (9683 12388764 AT
A); (9684 12388791 A G); (9685 12389518 T C); (9686 12389613 T C); (9687 12389765 G T); (9688
12389776 G A); (9689 12389777 T C); (9690 12389798 G A); (9691 12389972 A C); (9692 12389979 T
A); (9693 12390139 G A); (9694 12390631 G T); (9695 12390811 A G); (9696 12390938 G T); (9697
12391392 C T); (9698 12391612 C T); (9699 12391650 T C); (9700 12391986 A G); (9701 12392006 A
G); (9702 12392196 C T); (9703 12392213 A G); (9704 12393258 A G); (9705 12393361 C G); (9706
12393698 C T); (9707 12393705 T C); (9708 12393752 T G); (9709 12393755 A C); (9710 12393761 T
G); (9711 12394002 G A); (9712 12394234 G A); (9713 12394251 G T); (9714 12394305 G C); (9715
12394667 T C); (9716 12395175 A G); (9717 12395763 T C); (9718 12396621 G A); (9719 12396655 A
G); (9720 12396734 C T); (9721 12397061 T A); (9722 12397315 C G); (9723 12398236 T C); (9724
12399663 G T); (9725 12399838 T C); (9726 12400374 G A); (9727 12400574 T C); (9728 12400789 A
G); (9729 12401295 T C); (9730 12402193 T A); (9731 12402194 C T); (9732 12402332 G A); (9733
12402363 C A); (9734 12402369 G C); (9735 12402431 A G); (9736 12402507 G A); (9737 12402513 A
G); (9738 12402533 T C); (9739 12402957 A G); (9740 12402970 T C); (9741 12403001 C T); (9742
12403018 G A); (9743 12403040 C T); (9744 12403069 T C); (9745 12403070 T G); (9746 12403071 A
C); (9747 12403109 C T); (9748 12403172 C A); (9749 12403379 G A); (9750 12403496 G T); (9751
12403673 G A); (9752 12403682 A G); (9753 12404698 T C); (9754 12404811 T C); (9755 12404907 C
T); (9756 12404910 C T); (9757 12405099 T A); (9758 12405151 G A); (9759 12405170 A G); (9760
12405172 C T); (9761 12405573 G A); (9762 12406435 A C); (9763 12406445 G A); (9764 12406763 T
A); (9765 12406911 T A); (9766 12407068 A G); (9767 12407186 G A); (9768 12407419 G A); (9769
12407492 T C); (9770 12407498 T C); (9771 12407533 A G); (9772 12407576 AT A); (9773 12407595 G
A); (9774 12407743 T C); (9775 12409138 A G); (9776 12409510 A C); (9777 12410061 G A); (9778
12410233 C T); (9779 12410296 G A); (9780 12411181 T C); (9781 12411389 A C); (9782 12411431 C
T); (9783 12412091 A C); (9784 12412300 G A); (9785 12412617 A AC); (9786 12412686 C T); (9787
12412725 G A); (9788 12413408 T G); (9789 12413426 C CT); (9790 12413797 C T); (9791 12413975 T
C); (9792 12414248 G A); (9793 12415243 T C); (9794 12415346 C T); (9795 12415480 T C); (9796
12416299 T C); (9797 12416321 AC A); (9798 12416595 T A); (9799 12417238 A AG); (9800 12417326 G
A); (9801 12418453 G A); (9802 12418985 T G); (9803 12419179 A G); (9804 12419303 T G); (9805
12419397 C T); (9806 12419406 A G); (9807 12419430 A G); (9808 12419442 T C); (9809 12422065 T
G); (9810 12422123 A G); (9811 12422407 G A); (9812 12422622 A G); (9813 12423040 T G); (9814
12423571 G A); (9815 12423655 G A); (9816 12423816 C T); (9817 12424098 A G); (9818 12425517 G
A); (9819 12426300 A G); (9820 12426429 A G); (9821 12426474 G A); (9822 12426494 A G); (9823
12428516 T C); (9824 12428534 T C); (9825 12428553 A G); (9826 12428581 G A); (9827 12428620 G
A); (9828 12428643 A C); (9829 12429161 G A); (9830 12429207 T C); (9831 12430053 G T); (9832
12430400 A G); (9833 12430956 G T); (9834 12431112 C T); (9835 12431235 A G); (9836 12431923 C
T); (9837 12432586 A T); (9838 12432655 C T); (9839 12432790 T C); (9840 12433448 C T); (9841
12433490 T C); (9842 12433759 G A); (9843 12434002 G C); (9844 12434034 T C); (9845 12434753 G
C); (9846 12434793 A G); (9847 12434903 A G); (9848 12434924 A G); (9849 12435640 C T); (9850
12435669 T C); (9851 12435818 A C); (9852 12435930 C A); (9853 12436285 T C); (9854 12436327 T
C); (9855 12436468 T C); (9856 12436493 A G); (9857 12436541 A G); (9858 12436608 A C); (9859
12436609 A C); (9860 12437327 T C); (9861 12437415 A T); (9862 12437772 A G); (9863 12438092 T
C); (9864 12438230 C A); (9865 12438452 A G); (9866 12439305 A G); (9867 12439360 C T); (9868
12439568 A T); (9869 12439684 A G); (9870 12439686 C A); (9871 12439689 A T); (9872 12439709 G
A); (9873 12439734 A G); (9874 12439797 A G); (9875 12440271 T C); (9876 12440575 T C); (9877
12440645 G C); (9878 12442375 ATCGAT A); (9879 12442437 C T); (9880 12442597 G A); (9881
12443008 C A); (9882 12443118 T A); (9883 12443172 A G); (9884 12443406 G A); (9885 12444185 G
T); (9886 12444377 A C); (9887 12444455 T G); (9888 12444736 A AACACTATG); (9889 12445026 T
G); (9890 12445491 T C); (9891 12447219 T C); (9892 12447590 A T); (9893 12448253 C T); (9894
12448439 A T); (9895 12448673 A T); (9896 12448689 G A); (9897 12448840 C T); (9898 12449423 T
C); (9899 12449948 G A); (9900 12450497 C G); (9901 12451333 T A); (9902 12451336 A G); (9903
12451343 A G); (9904 12451355 T G); (9905 12451625 A T); (9906 12451660 A G); (9907 12453478 C
T); (9908 12453505 G A); (9909 12453506 T C); (9910 12453706 A G); (9911 12455100 C T); (9912
12456187 A G); (9913 12456234 A G); (9914 12456289 G A); (9915 12457227 T A); (9916 12457273 A
G); (9917 12457346 A G); (9918 12457372 G A); (9919 12459758 C T); (9920 12460416 C A); (9921
12460914 C T); (9922 12461780 AC A); (9923 12461964 G A); (9924 12462000 G A); (9925 12464403 C
T); (9926 12464599 CT C); (9927 12465583 A G); (9928 12466232 C A); (9929 12466655 T G); (9930
12466656 A G); (9931 12466722 G T); (9932 12467121 A G); (9933 12467259 T G); (9934 12467281 C
G); (9935 12467700 A G); (9936 12467803 C T); (9937 12467833 G A); (9938 12468277 C T); (9939
12468358 T C); (9940 12468598 AC A); (9941 12468632 G A); (9942 12468766 A C); (9943 12468987 A
T); (9944 12469396 A G); (9945 12469994 T G); (9946 12470507 A G); (9947 12471193 G A); (9948
12471438 T C); (9949 12471458 TTGA T); (9950 12471485 C A); (9951 12471853 C T); (9952 12471937 T
C); (9953 12472024 T C); (9954 12472267 G A); (9955 12472415 T C); (9956 12472503 A G); (9957
12472595 T C); (9958 12472741 C T); (9959 12473457 A AT); (9960 12473877 T A); (9961 12473879 G
A); (9962 12473903 T A); (9963 12473979 A G); (9964 12474668 T G); (9965 12474707 T C); (9966
12474829 C G); (9967 12475060 A G); (9968 12476409 C G); (9969 12476622 C T); (9970 12476657 T
G); (9971 12476855 A G); (9972 12476953 G A); (9973 12477716 A C); (9974 12477909 T G); (9975
12478449 G A); (9976 12479362 C T); (9977 12479429 A G); (9978 12479730 T A); (9979 12480038 C
T); (9980 12480104 A AAC); (9981 12480309 T G); (9982 12480525 C T); (9983 12480607 G T); (9984
12481033 T C); (9985 12481131 T C); (9986 12481223 T G); (9987 12481386 A G); (9988 12481931 T
G); (9989 12482066 C T); (9990 12482189 A C); (9991 12482365 G A); (9992 12482366 C T); (9993
12482370 G A); (9994 12482450 G A); (9995 12482855 G A); (9996 12482909 T A); (9997 12482941 G
A); (9998 12483094 T C); (9999 12483157 G T); (10000 12483196 G A); (10001 12483388 A G); (10002
12483604 A G); (10003 12483645 T G); (10004 12483792 C T); (10005 12484211 G C); (10006 12484373
A G); (10007 12484533 G A); (10008 12484626 C T); (10009 12484716 A G); (10010 12484783 C
T); (10011 12485233 G A); (10012 12485874 T C); (10013 12486141 T C); (10014 12486266 A G); (10015
12486396 T C); (10016 12486457 T C); (10017 12486638 CA C); (10018 12486701 C T); (10019 12486791
C T); (10020 12486974 A AT); (10021 12487039 G A); (10022 12487109 G A); (10023 12487126 C
T); (10024 12487166 C T); (10025 12487299 C T); (10026 12487343 T C); (10027 12487579 G A); (10028
12487656 T C); (10029 12487709 G A); (10030 12487819 AAT A); (10031 12488343 G A); (10032
12489015 C T); (10033 12489174 G T); (10034 12489294 A G); (10035 12489516 C T); (10036 12489517
A G); (10037 12489529 C G); (10038 12489550 G A); (10039 12489649 A G); (10040 12489701 T
C); (10041 12489721 C T); (10042 12489761 A G); (10043 12489762 C T); (10044 12489960 T C); (10045
12490013 G A); (10046 12490099 C G); (10047 12490149 G A); (10048 12490235 CAGG C); (10049
12490465 T A); (10050 12490626 A G); (10051 12490765 T C); (10052 12490841 G A); (10053 12491406
T G); (10054 12491945 C T); (10055 12492202 G A); (10056 12492363 A G); (10057 12492781 A
C); (10058 12493525 T G); (10059 12493910 G A); (10060 12494134 G T); (10061 12494172 G A); (10062
12494288 G T); (10063 12494462 G GA); (10064 12495632 T C); (10065 12495844 T A); (10066
12496334 T A); (10067 12496366 A T); (10068 12496427 C A); (10069 12496438 A G); (10070 12496485
C T); (10071 12496529 G C); (10072 12496636 T C); (10073 12497559 C G); (10074 12497799 T C); (10075
12499937 GA G); (10076 12501088 C T); (10077 12501177 C T); (10078 12502170 T C); (10079 12502505
G A); (10080 12502534 A C); (10081 12502588 G A); (10082 12502927 C T); (10083 12503033 T
G); (10084 12503909 T G); (10085 12504064 C T); (10086 12504722 G A); (10087 12504984 C T); (10088
12505236 C T); (10089 12505429 G A); (10090 12505505 C T); (10091 12505943 T G); (10092 12506139
G A); (10093 12506988 A G); (10094 12507422 C T); (10095 12507533 A G); (10096 12507538 C
T); (10097 12507614 T A); (10098 12507906 G A); (10099 12508184 A G); (10100 12508302 C T); (10101
12508413 A G); (10102 12508524 C G); (10103 12508733 AAGC A); (10104 12508772 T G); (10105
12509686 A G); (10106 12510609 G T); (10107 12510705 T G); (10108 12511072 C T); (10109 12511171
A T); (10110 12511345 G A); (10111 12511382 A G); (10112 12511417 G A); (10113 12511442 T
C); (10114 12511462 T C); (10115 12511550 A G); (10116 12511576 T C); (10117 12512786 T C); (10118
12512885 C T); (10119 12513203 A G); (10120 12513291 T C); (10121 12513480 A G); (10122 12513796
T A); (10123 12514035 T A); (10124 12514071 T C); (10125 12514162 A G); (10126 12514166 C T); (10127
12514609 G A); (10128 12514645 T C); (10129 12515008 T C); (10130 12515151 G A); (10131 12515278
A G); (10132 12515499 A AG); (10133 12516057 G A); (10134 12517109 T C); (10135 12517309 G
A); (10136 12517327 T A); (10137 12517882 G T); (10138 12518091 G A); (10139 12518301 T C); (10140
12518324 A T); (10141 12518667 C T); (10142 12519041 T C); (10143 12519418 C T); (10144 12519891 C
A); (10145 12519924 A G); (10146 12520283 GA G); (10147 12520473 A G); (10148 12520580 C
A); (10149 12520766 C T); (10150 12520827 C A); (10151 12520907 A C); (10152 12521106 C T); (10153
12521326 T G); (10154 12521803 A G); (10155 12521884 A G); (10156 12521990 T C); (10157 12522500
C T); (10158 12522788 C CT); (10159 12522906 C T); (10160 12522980 T C); (10161 12523589 T
C); (10162 12523624 G A); (10163 12524031 A T); (10164 12524809 C T); (10165 12524889 C T); (10166
12525320 A G); (10167 12525450 A G); (10168 12525591 T C); (10169 12525613 G A); (10170 12525669
A C); (10171 12525759 C T); (10172 12525813 G A); (10173 12526145 CCATATTGAAA C); (10174
12526434 A G); (10175 12526624 T C); (10176 12526641 A G); (10177 12526654 T C); (10178 12526735
A G); (10179 12527272 C T); (10180 12527522 G A); (10181 12527557 G A); (10182 12527666 A
G); (10183 12527708 G A); (10184 12527918 G A); (10185 12528052 T A); (10186 12528198 G A); (10187
12529121 C T); (10188 12530668 A C); (10189 12530744 G C); (10190 12530783 G C); (10191 12531094
T C); (10192 12531155 GT G); (10193 12531184 G A); (10194 12532447 T C); (10195 12532518 T
G); (10196 12532908 G T); (10197 12532974 G A); (10198 12533054 T C); (10199 12533065 GA
G); (10200 12533073 C T); (10201 12533112 A AT); (10202 12535088 C A); (10203 12536050 A G); (10204
12536213 A G); (10205 12538014 GT G); (10206 12538735 A G); (10207 12538870 TA T); (10208
12538888 AT A); (10209 12539001 C T); (10210 12539136 A C); (10211 12539537 C A); (10212 12539579
A G); (10213 12539845 A G); (10214 12539883 A G); (10215 12539991 G A); (10216 12540052 A
G); (10217 12540077 A G); (10218 12541186 C T); (10219 12541231 A T); (10220 12541333 C T); (10221
12541874 C T); (10222 12541908 G A); (10223 12541968 A G); (10224 12541978 G A); (10225 12542038
T G); (10226 12542143 T G); (10227 12542330 G T); (10228 12544316 G A); (10229 12545777 A
G); (10230 12545874 C A); (10231 12548722 T A); (10232 12548740 T C); (10233 12549725 T C); (10234
12551985 G A); (10235 12552149 A G); (10236 12552316 A G); (10237 12552394 G A); (10238 12552408
G T); (10239 12552672 T C); (10240 12552983 T C); (10241 12552984 T C); (10242 12552991 T C); (10243
12553312 C T); (10244 12553383 T C); (10245 12553459 A G); (10246 12553791 T G); (10247 12553869
AT A); (10248 12554394 T C); (10249 12554513 C T); (10250 12554535 C G); (10251 12554783 A
T); (10252 12555008 T A); (10253 12555241 C A); (10254 12556151 C T); (10255 12556181 G A); (10256
12556215 T A); (10257 12556226 T C); (10258 12556284 T C); (10259 12556909 C T); (10260 12556976 C
G); (10261 12557203 T C); (10262 12557325 T C); (10263 12557378 T C); (10264 12558891 G T); (10265
12558893 G T); (10266 12558975 C A); (10267 12559295 C T); (10268 12560190 T C); (10269 12560271 C
G); (10270 12560309 T A); (10271 12560383 C A); (10272 12560388 T C); (10273 12560760 C A); (10274
12561046 G A); (10275 12561571 G A); (10276 12561842 A C); (10277 12561844 A C); (10278 12561846
A C); (10279 12561848 A C); (10280 12561920 A G); (10281 12562043 T C); (10282 12562158 A
G); (10283 12562359 T C); (10284 12562929 C A); (10285 12563176 T C); (10286 12565667 A G); (10287
12567345 A G); (10288 12567470 A C); (10289 12567608 A G); (10290 12567790 A G); (10291 12567814
G A); (10292 12568040 C T); (10293 12568246 T C); (10294 12568791 G A); (10295 12568800 G
A); (10296 12568841 G T); (10297 12568918 C T); (10298 12569449 G A); (10299 12569453 A G); (10300
12570129 T C); (10301 12570247 A G); (10302 12571090 C T); (10303 12571343 G C); (10304 12571638
C T); (10305 12572055 G C); (10306 12572576 A C); (10307 12573076 A G); (10308 12573759 A
C); (10309 12574217 A G); (10310 12574829 T G); (10311 12574980 G C); (10312 12575138 C T); (10313
12575196 A G); (10314 12575668 G GAAGGTCGAAAATGAATTA); (10315 12575888 G C); (10316
12576740 C T); (10317 12577169 G A); (10318 12577970 C A); (10319 12578728 G A); (10320 12579001
A G); (10321 12579126 C G); (10322 12579608 G C); (10323 12580174 A G); (10324 12580404 C
A); (10325 12580420 A T); (10326 12580700 T G); (10327 12581408 A G); (10328 12581499 T A); (10329
12581834 G T); (10330 12581929 T C); (10331 12582074 A G); (10332 12582287 T G); (10333 12583013
AT A); (10334 12583102 A ATGTGTTTC); (10335 12583236 T A); (10336 12583278 C T); (10337 12583352
T C); (10338 12583999 T C); (10339 12584235 A G); (10340 12584364 A G); (10341 12584501 TG
T); (10342 12584574 A G); (10343 12584957 T C); (10344 12585912 C T); (10345 12585951 C T); (10346
12586856 T C); (10347 12586968 G A); (10348 12587911 C T); (10349 12588066 T C); (10350 12588680
G A); (10351 12588681 A G); (10352 12589266 A G); (10353 12589619 A C); (10354 12589675 A
G); (10355 12589830 C T); (10356 12590327 T A); (10357 12591338 C T); (10358 12591587 G T); (10359
12591795 G A); (10360 12591947 T G); (10361 12592065 T C); (10362 12592212 A G); (10363 12592783
T A); (10364 12592797 A T); (10365 12593850 C G); (10366 12594112 A G); (10367 12594319 A
T); (10368 12594386 G C); (10369 12594785 C T); (10370 12594923 G A); (10371 12594937 T C); (10372
12594968 A G); (10373 12595208 T C); (10374 12595254 T G); (10375 12595257 AAG A); (10376
12595804 A G); (10377 12596075 T C); (10378 12596115 G A); (10379 12596321 C T); (10380 12596541
T C); (10381 12597096 T C); (10382 12597529 C T); (10383 12597545 C T); (10384 12598193 C A); (10385
12598217 G A); (10386 12598442 G A); (10387 12598503 A G); (10388 12598626 A G); (10389 12598639
A C); (10390 12598726 T C); (10391 12598848 G A); (10392 12599383 G A); (10393 12600004 G
A); (10394 12601089 T G); (10395 12601146 G A); (10396 12601336 T C); (10397 12601469 T C); (10398
12601794 T A); (10399 12602010 T A); (10400 12602188 C A); (10401 12602806 G GA); (10402
12602874 C A); (10403 12603548 A C); (10404 12603617 G A); (10405 12603697 A AT); (10406
12603711 G T); (10407 12603928 A G); (10408 12603988 T G); (10409 12604465 C G); (10410 12604881
A AT); (10411 12605616 T C); (10412 12606352 G A); (10413 12606596 C A); (10414 12606870 A
T); (10415 12606928 G A); (10416 12607051 T C); (10417 12607146 C A); (10418 12607301 A T); (10419
12607344 G A); (10420 12607571 A C); (10421 12607642 A G); (10422 12608747 TC T); (10423
12608756 A C); (10424 12608841 C T); (10425 12609015 T A); (10426 12609102 T A); (10427 12609626
G A); (10428 12610006 T C); (10429 12610067 T C); (10430 12610095 A C); (10431 12610112 AT
A); (10432 12610274 A G); (10433 12611135 G A); (10434 12611291 G A); (10435 12611558 C T); (10436
12611626 G A); (10437 12611783 A G); (10438 12611800 T A); (10439 12612165 A G); (10440 12612427
A G); (10441 12612791 T C); (10442 12613212 T C); (10443 12613509 A G); (10444 12613831 A
T); (10445 12615097 T C); (10446 12615108 T C); (10447 12615116 G T); (10448 12615659 T A); (10449
12615685 C T); (10450 12616376 G A); (10451 12617423 C T); (10452 12617841 C T); (10453 12617843 T
C); (10454 12617995 C T); (10455 12618238 T C); (10456 12618288 T TC); (10457 12618453 C T); (10458
12618524 C T); (10459 12619028 T C); (10460 12619045 A G); (10461 12619216 T C); (10462 12619284 C
T); (10463 12619429 A G); (10464 12619431 C T); (10465 12619567 G A); (10466 12619734 C A); (10467
12619877 G A); (10468 12619989 G A); (10469 12620232 T G); (10470 12621767 A T); (10471 12621787
T C); (10472 12621790 T C); (10473 12622039 A G); (10474 12622152 G A); (10475 12622172 T
TTATTTTC); (10476 12622905 A G); (10477 12622917 C T); (10478 12622943 T C); (10479 12622961 A
G); (10480 12623319 T C); (10481 12624627 G T); (10482 12625220 G A); (10483 12626036 A T); (10484
12626173 T A); (10485 12626801 T C); (10486 12627188 C T); (10487 12627240 G A); (10488 12627245
A G); (10489 12627355 C T); (10490 12627682 A G); (10491 12627776 A G); (10492 12627953 G
T); (10493 12628533 T C); (10494 12628556 C T); (10495 12628557 A G); (10496 12629041 T A); (10497
12629797 C T); (10498 12629951 C T); (10499 12629998 G A); (10500 12630772 G T); (10501 12631115
G A); (10502 12631478 C T); (10503 12631796 C T); (10504 12632027 A C); (10505 12632041 G
A); (10506 12632434 C T); (10507 12632510 A G); (10508 12632667 C T); (10509 12633395 A G); (10510
12633768 A C); (10511 12633931 G A); (10512 12634448 C A); (10513 12634844 A G); (10514 12634858
T C); (10515 12634892 T C); (10516 12635043 TG T); (10517 12635064 A T); (10518 12635182 T
C); (10519 12635231 G T); (10520 12635583 T C); (10521 12635648 T C); (10522 12635776 AAG
A); (10523 12635803 A G); (10524 12637103 G C); (10525 12637256 C T); (10526 12637396 CT C); (10527
12637551 C A); (10528 12638656 T C); (10529 12639029 A G); (10530 12639210 C T); (10531 12639298
G T); (10532 12639599 C A); (10533 12639679 C T); (10534 12639781 C T); (10535 12640041 T C); (10536
12640417 A G); (10537 12640495 A G); (10538 12640708 G A); (10539 12640719 T C); (10540 12641447
T C); (10541 12641665 G A); (10542 12641811 C T); (10543 12642874 T C); (10544 12643497 G T); (10545
12643737 T C); (10546 12643745 T C); (10547 12644077 T C); (10548 12644408 C T); (10549 12644697 G
A); (10550 12644759 T C); (10551 12646655 A G); (10552 12646758 T C); (10553 12647680 G A); (10554
12647844 T C); (10555 12647862 T C); (10556 12648711 A G); (10557 12648712 A G); (10558 12648714
A G); (10559 12648720 A G); (10560 12648786 A G); (10561 12650707 C A); (10562 12653051 G
A); (10563 12653056 G A); (10564 12655805 G A); (10565 12655916 C A); (10566 12656801 G T); (10567
12656859 T TTA); (10568 12659257 C T); (10569 12661188 A C); (10570 12661324 T C); (10571
12666964 G T); (10572 12667603 T C); (10573 12667998 C T); (10574 12668023 C T); (10575 12668775
G A); (10576 12668798 A G); (10577 12669948 C A); (10578 12670591 C A); (10579 12670790 G
A); (10580 12671424 T C); (10581 12671639 T C); (10582 12671832 C T); (10583 12671978 C
CAT); (10584 12671993 T C); (10585 12672504 T C); (10586 12672628 A C); (10587 12672759 T
C); (10588 12672766 C G); (10589 12672932 AAG A); (10590 12673007 A G); (10591 12673010 T
C); (10592 12673041 C T); (10593 12673090 T C); (10594 12673182 A G); (10595 12673191 G A); (10596
12673274 C T); (10597 12673519 C T); (10598 12673642 C T); (10599 12673845 G A); (10600 12673999 T
C); (10601 12674029 G A); (10602 12674048 A T); (10603 12674315 GTTCAC G); (10604 12674396 A
G); (10605 12674523 G A); (10606 12674525 T C); (10607 12674641 T TGA); (10608 12674668 A
G); (10609 12674743 C T); (10610 12674891 T A); (10611 12674935 A G); (10612 12675184 C T); (10613
12675198 A G); (10614 12675431 G C); (10615 12675444 A G); (10616 12675824 C G); (10617 12675834
G T); (10618 12676060 G A); (10619 12676612 T C); (10620 12676617 C A); (10621 12676832 A
G); (10622 12677162 G A); (10623 12677164 T C); (10624 12677242 A G); (10625 12677248 A G); (10626
12677413 A G); (10627 12678423 C T); (10628 12678437 C G); (10629 12678475 C T); (10630 12680065
T G); (10631 12680088 C T); (10632 12680099 A G); (10633 12680452 T G); (10634 12680514 A
G); (10635 12680537 T G); (10636 12680558 G T); (10637 12680561 G A); (10638 12680572 C T); (10639
12680707 A G); (10640 12681224 G A); (10641 12681507 T C); (10642 12682083 C T); (10643 12682107
G A); (10644 12682113 T C); (10645 12682468 T C); (10646 12683091 A G); (10647 12683264 C
T); (10648 12683293 G A); (10649 12683878 C A); (10650 12684274 C T); (10651 12684275 T C); (10652
12684298 G A); (10653 12684360 C T); (10654 12684740 G A); (10655 12684854 A G); (10656 12684924
C G); (10657 12685033 T C); (10658 12685783 A G); (10659 12686103 C G); (10660 12686203 A
T); (10661 12686461 G A); (10662 12687064 G A); (10663 12687428 C T); (10664 12687662 G A); (10665
12687785 A G); (10666 12687947 C T); (10667 12687990 T C); (10668 12688111 C T); (10669 12688361 T
C); (10670 12688370 G GA); (10671 12688373 C T); (10672 12688464 T C); (10673 12688473 C T); (10674
12688519 T C); (10675 12688595 T C); (10676 12688696 G A); (10677 12688711 C G); (10678 12688793
T C); (10679 12689178 A G); (10680 12689190 A G); (10681 12689581 T C); (10682 12689594 G
A); (10683 12689698 T C); (10684 12689737 G A); (10685 12689750 C T); (10686 12690057 C T); (10687
12690072 C T); (10688 12690146 G A); (10689 12690172 C T); (10690 12690514 A G); (10691 12691970
G A); (10692 12692188 G A); (10693 12692789 A C); (10694 12692806 C T); (10695 12692815 T
G); (10696 12692816 T C); (10697 12692826 A G); (10698 12692836 A C); (10699 12692847 T G); (10700
12692975 C T); (10701 12695766 T C); (10702 12695805 C T); (10703 12695831 T A); (10704 12696125 T
C); (10705 12696132 C T); (10706 12701989 T C); (10707 12706341 G A); (10708 12711594 C T); (10709
12711676 AAC A); (10710 12711845 A C); (10711 12711934 A C); (10712 12712111 G A); (10713
12712522 A G); (10714 12712688 G A); (10715 12714311 C T); (10716 12714413 C G); (10717 12714485
T C); (10718 12714518 T G); (10719 12715476 T C); (10720 12715583 C G); (10721 12715601 A
AG); (10722 12715779 A C); (10723 12716136 A G); (10724 12716319 T A); (10725 12716367 G
A); (10726 12718191 G A); (10727 12718515 TAA T); (10728 12718851 G A); (10729 12720279 T
C); (10730 12720682 A T); (10731 12720930 T A); (10732 12721135 G A); (10733 12721211 G T); (10734
12721609 C T); (10735 12721681 A G); (10736 12722485 A T); (10737 12722608 A G); (10738 12723483
T C); (10739 12723546 C A); (10740 12723964 G A); (10741 12723983 C T); (10742 12724985 T
G); (10743 12725276 C T); (10744 12725594 C T); (10745 12725655 G A); (10746 12725709 T C); (10747
12725717 A G); (10748 12725818 T G); (10749 12725841 G A); (10750 12725888 C T); (10751 12725897
C T); (10752 12725899 A G); (10753 12726569 G A); (10754 12726649 T C); (10755 12727040 A
G); (10756 12727582 T C); (10757 12727929 G A); (10758 12728330 G T); (10759 12729973 C T); (10760
12730265 A G); (10761 12731918 T C); (10762 12731940 T C); (10763 12732038 A G); (10764 12732064
C T); (10765 12732403 C T); (10766 12732430 T C); (10767 12732451 T C); (10768 12732484 A C); (10769
12733154 A C); (10770 12733852 T C); (10771 12734102 G A); (10772 12734153 G A); (10773 12734509
T A); (10774 12734836 G A); (10775 12735059 A G); (10776 12735439 T C); (10777 12735444 G
A); (10778 12735587 A G); (10779 12735593 C T); (10780 12735874 C T); (10781 12736248 G A); (10782
12736293 A G); (10783 12736388 G A); (10784 12736515 G A); (10785 12736536 A G); (10786 12736793
C T); (10787 12736987 A C); (10788 12737398 A C); (10789 12737548 T C); (10790 12737752 A
G); (10791 12738381 G A); (10792 12738494 T C); (10793 12738689 G A); (10794 12738705 C A); (10795
12738860 C A); (10796 12739609 T C); (10797 12739812 C A); (10798 12739973 G C); (10799 12740372
A G); (10800 12742326 C T); (10801 12742411 G A); (10802 12742488 G A); (10803 12742573 G
A); (10804 12742622 C T); (10805 12746258 C T); (10806 12746374 C T); (10807 12746405 C T); (10808
12746596 G A); (10809 12750041 A G); (10810 12752654 C T); (10811 12752739 A G); (10812 12752922
T C); (10813 12752945 T A); (10814 12770364 G A); (10815 12772610 A G); (10816 12772937 A
T); (10817 12773022 C T); (10818 12773159 A G); (10819 12773391 A G); (10820 12773452 C T); (10821
12773464 A C); (10822 12773515 T A); (10823 12773569 G C); (10824 12773612 C G); (10825 12773646
C T); (10826 12773665 C T); (10827 12774992 T C); (10828 12775039 A G); (10829 12775118 A C); (10830
12775194 C CT); (10831 12776457 C A); (10832 12776606 G A); (10833 12776682 C T); (10834 12776696
A T); (10835 12776945 G A); (10836 12776987 A G); (10837 12777060 A G); (10838 12777068 G
A); (10839 12777256 C T); (10840 12778233 A ATGCT); (10841 12778369 G A); (10842 12778394 G
A); (10843 12778462 C T); (10844 12778489 G T); (10845 12778732 C G); (10846 12778937 A C); (10847
12779130 G C); (10848 12779131 A G); (10849 12779157 C G); (10850 12779635 C T); (10851 12779751
A G); (10852 12779867 A C); (10853 12780197 A T); (10854 12780216 T G); (10855 12780244 T
C); (10856 12781083 G A); (10857 12781107 C T); (10858 12781115 G A); (10859 12781135 G A); (10860
12781195 G A); (10861 12781226 A G); (10862 12781323 A G); (10863 12781462 C T); (10864 12781590
C T); (10865 12781693 T A); (10866 12781818 T G); (10867 12782348 T G); (10868 12782488 G
C); (10869 12782514 T C); (10870 12782780 A G); (10871 12782803 T C); (10872 12783945 C T); (10873
12783984 T C); (10874 12784016 C T); (10875 12784213 C T); (10876 12784459 T C); (10877 12784571 C
T); (10878 12784772 G A); (10879 12785051 T C); (10880 12785109 A G); (10881 12785810 C T); (10882
12786530 T C); (10883 12786557 A G); (10884 12786802 G C); (10885 12786804 G C); (10886 12786864
C G); (10887 12786971 C A); (10888 12787192 G C); (10889 12788767 G A); (10890 12789058 G
A); (10891 12789977 T A); (10892 12790875 T C); (10893 12790886 C T); (10894 12790923 G A); (10895
12791059 T G); (10896 12791082 T A); (10897 12791525 G C); (10898 12792098 A C); (10899 12792529
T TA); (10900 12793039 T G); (10901 12793624 G A); (10902 12793846 G A); (10903 12793987 A
G); (10904 12794140 G A); (10905 12795033 T C); (10906 12795098 C A); (10907 12795107 C T); (10908
12796921 T A); (10909 12797050 G A); (10910 12797180 CA C); (10911 12797237 A G); (10912
12797340 T C); (10913 12797351 C A); (10914 12797651 A G); (10915 12797912 T G); (10916 12798017
A G); (10917 12798556 A G); (10918 12798557 C T); (10919 12799043 A G); (10920 12799258 T
C); (10921 12799280 G T); (10922 12799414 T C); (10923 12800174 A G); (10924 12800546 T C); (10925
12801291 T C); (10926 12801361 C T); (10927 12801375 C T); (10928 12801376 G A); (10929 12801435 C
T); (10930 12801441 T C); (10931 12801448 T C); (10932 12801537 G A); (10933 12801674 C T); (10934
12801702 G A); (10935 12801703 T C); (10936 12801734 T C); (10937 12802007 A T); (10938 12802030
A C); (10939 12802184 T C); (10940 12802429 G A); (10941 12802847 G T); (10942 12803020 G
A); (10943 12803366 C T); (10944 12803691 C T); (10945 12804111 G A); (10946 12804615 G T); (10947
12804798 T C); (10948 12805032 G A); (10949 12805126 G A); (10950 12806244 G GA); (10951
12807072 G A); (10952 12807947 A G); (10953 12807979 G A); (10954 12808833 G A); (10955 12810903
C G); (10956 12811016 T C); (10957 12811053 A G); (10958 12812026 T C); (10959 12812052 G
A); (10960 12812104 GA G); (10961 12813214 T C); (10962 12813424 T C); (10963 12813446 T C); (10964
12814643 G A); (10965 12816163 G A); (10966 12818516 C A); (10967 12819540 T G); (10968 12819596
G A); (10969 12819685 C T); (10970 12820609 C T); (10971 12820743 C A); (10972 12822964 C T); (10973
12823061 G A); (10974 12823432 A C); (10975 12823500 C T); (10976 12823726 G A); (10977 12823904
G T); (10978 12824061 G T); (10979 12824080 G C); (10980 12824614 C T); (10981 12824955 C
G); (10982 12825006 C T); (10983 12825271 A G); (10984 12825288 T C); (10985 12825316 G A); (10986
12825505 A G); (10987 12825584 G A); (10988 12826208 G A); (10989 12826218 A C); (10990 12826271
T C); (10991 12826282 T C); (10992 12826512 T G); (10993 12826532 T A); (10994 12828881 G A); (10995
12828981 A G); (10996 12828995 A G); (10997 12829093 TG T); (10998 12829311 G A); (10999
12829378 T G); (11000 12829426 T C); (11001 12830087 G T); (11002 12830110 G T); (11003 12830142
C T); (11004 12830143 C A); (11005 12830488 G T); (11006 12830497 G A); (11007 12830502 A
G); (11008 12830516 A G); (11009 12830797 A G); (11010 12830854 T C); (11011 12830968 T C); (11012
12831088 G A); (11013 12831586 T G); (11014 12831784 T A); (11015 12832925 C T); (11016 12833057
G A); (11017 12833310 T A); (11018 12833343 A G); (11019 12833510 G A); (11020 12833551 C
T); (11021 12833565 C G); (11022 12834025 A C); (11023 12834095 G A); (11024 12834099 T A); (11025
12834101 G A); (11026 12835311 C G); (11027 12835446 T C); (11028 12835454 T C); (11029 12835475
C T); (11030 12835514 C T); (11031 12835640 C T); (11032 12835871 T C); (11033 12835877 G T); (11034
12835900 A G); (11035 12837879 G A); (11036 12837887 T C); (11037 12837982 A G); (11038 12837998
C T); (11039 12838007 T C); (11040 12838015 C A); (11041 12838032 A G); (11042 12838212 C T); (11043
12838268 A C); (11044 12838441 A T); (11045 12838458 G A); (11046 12838481 T C); (11047 12838602
A G); (11048 12838652 T A); (11049 12838691 T C); (11050 12838714 A T); (11051 12838816 C T); (11052
12839200 C A); (11053 12839209 G A); (11054 12839381 C T); (11055 12839384 T C); (11056 12839417
G A); (11057 12840415 T A); (11058 12840513 A G); (11059 12840704 A G); (11060 12840914 G
A); (11061 12840995 A G); (11062 12841014 A G); (11063 12841119 G A); (11064 12841264 C G); (11065
12841451 A C); (11066 12841937 A G); (11067 12842014 T C); (11068 12842121 A G); (11069 12842142
A G); (11070 12842472 T C); (11071 12842595 G T); (11072 12842842 C T); (11073 12842857 G
A); (11074 12842972 G A); (11075 12843291 G C); (11076 12843318 T C); (11077 12843710 T A); (11078
12843904 A C); (11079 12843914 C T); (11080 12844290 G T); (11081 12844856 G T); (11082 12844907
G A); (11083 12845172 C T); (11084 12845842 G A); (11085 12846015 T C); (11086 12846128 A
C); (11087 12846206 G T); (11088 12846247 C T); (11089 12846265 T G); (11090 12846704 C T); (11091
12846802 C T); (11092 12846875 G A); (11093 12846898 C T); (11094 12847087 A G); (11095 12847359
A ACTCCTTGT); (11096 12848552 C G); (11097 12849416 C T); (11098 12852553 T C); (11099 12852752
G A); (11100 12853077 A C); (11101 12853135 C T); (11102 12853279 A G); (11103 12853932 G
A); (11104 12853935 C A); (11105 12854101 T C); (11106 12854322 A G); (11107 12854563 G A); (11108
12854652 A G); (11109 12854729 C A); (11110 12855261 C A); (11111 12855266 C T); (11112 12855280
C T); (11113 12855369 A C); (11114 12855931 G A); (11115 12855982 C T); (11116 12856218 T C); (11117
12856609 T A); (11118 12856626 G A); (11119 12856826 C G); (11120 12856846 A G); (11121 12856862
T C); (11122 12857075 G A); (11123 12857650 T C); (11124 12857684 T C); (11125 12857964 C T); (11126
12858183 A AG); (11127 12858384 C T); (11128 12858386 C T); (11129 12858563 T C); (11130 12858610
A G); (11131 12858816 A G); (11132 12859105 T G); (11133 12859474 A C); (11134 12859607 C
A); (11135 12859824 A G); (11136 12860941 G A); (11137 12860999 A G); (11138 12861123 T G); (11139
12861416 A T); (11140 12861667 T G); (11141 12861821 A G); (11142 12861878 G C); (11143 12862579
T C); (11144 12862967 T C); (11145 12862989 G A); (11146 12866190 C T); (11147 12866835 C A); (11148
12866848 G A); (11149 12867202 G A); (11150 12867248 A G); (11151 12867332 G A); (11152 12867784
A G); (11153 12867924 T C); (11154 12868592 C T); (11155 12868610 A C); (11156 12868673 T C); (11157
12869000 C T); (11158 12869209 C A); (11159 12869433 T C); (11160 12869743 C G); (11161 12869807 T
C); (11162 12869886 G A); (11163 12870133 T G); (11164 12870402 A G); (11165 12870409 C T); (11166
12870738 G A); (11167 12870804 A C); (11168 12870960 A C); (11169 12871502 C T); (11170 12872062
G A); (11171 12872227 T G); (11172 12872490 G A); (11173 12872614 G A); (11174 12872707 G
A); (11175 12873458 C T); (11176 12873507 A G); (11177 12873790 A G); (11178 12873860 T C); (11179
12874055 G A); (11180 12874136 A G); (11181 12874362 G A); (11182 12875465 C G); (11183 12875686
G T); (11184 12875744 T A); (11185 12875836 A G); (11186 12876159 A T); (11187 12876319 C
A); (11188 12876667 T C); (11189 12878004 A C); (11190 12878637 A G); (11191 12879094 A G); (11192
12881990 A AC); (11193 12881998 G A); (11194 12882942 C G); (11195 12883592 A C); (11196
12883596 A G); (11197 12883663 T C); (11198 12884403 C T); (11199 12884446 A G); (11200 12884691
A G); (11201 12885040 A C); (11202 12885299 G A); (11203 12885374 C T); (11204 12885602 G
A); (11205 12885969 T C); (11206 12886040 T A); (11207 12886067 T C); (11208 12886825 C G); (11209
12886864 G T); (11210 12887030 C T); (11211 12887291 C T); (11212 12887351 T C); (11213 12887414
G T); (11214 12887425 T G); (11215 12887491 T G); (11216 12887526 G A); (11217 12887697 A
C); (11218 12887756 C A); (11219 12888402 T C); (11220 12888964 C T); (11221 12889594 G A); (11222
12889633 C T); (11223 12889763 C T); (11224 12889771 C T); (11225 12889854 C T); (11226 12890137 C
T); (11227 12891011 C T); (11228 12891029 A C); (11229 12891114 T C); (11230 12891328 T A); (11231
12891420 A G); (11232 12892008 C T); (11233 12892032 C T); (11234 12892042 T C); (11235 12892172
G A); (11236 12892471 T C); (11237 12892491 T C); (11238 12892552 T C); (11239 12892555 T C); (11240
12892675 G A); (11241 12892774 G A); (11242 12892800 T C); (11243 12892917 G A); (11244 12892955
C T); (11245 12892985 G A); (11246 12893084 C T); (11247 12893218 C G); (11248 12893264 A
C); (11249 12893374 T C); (11250 12894142 A G); (11251 12894447 G A); (11252 12894484 A G); (11253
12895138 T G); (11254 12895603 A G); (11255 12895613 T G); (11256 12895919 G A); (11257 12896248
C T); (11258 12896957 A G); (11259 12896959 A G); (11260 12897455 G C); (11261 12897526 G
A); (11262 12898294 C T); (11263 12898510 T C); (11264 12898570 G A); (11265 12898631 A G); (11266
12898984 C T); (11267 12899118 A T); (11268 12899140 C A); (11269 12899228 C T); (11270 12899275 T
A); (11271 12899765 G A); (11272 12899777 A G); (11273 12899884 A G); (11274 12899918 C T); (11275
12900011 G A); (11276 12900013 T C); (11277 12900079 T G); (11278 12900134 A G); (11279 12900311
A G); (11280 12900391 A G); (11281 12900506 T G); (11282 12901368 T C); (11283 12901384 T
C); (11284 12902103 A G); (11285 12902283 C A); (11286 12902284 T C); (11287 12902345 G T); (11288
12902375 C T); (11289 12902419 A G); (11290 12902551 C A); (11291 12902785 G A); (11292 12903247
C T); (11293 12903559 G A); (11294 12904082 G A); (11295 12904206 C T); (11296 12904267 A
G); (11297 12904326 G A); (11298 12904641 G A); (11299 12904700 T TC); (11300 12904890 G
A); (11301 12904894 G A); (11302 12904965 G A); (11303 12905231 G A); (11304 12905410 T C); (11305
12905458 G A); (11306 12905535 G A); (11307 12905606 C T); (11308 12905769 G T); (11309 12906033
G A); (11310 12906043 C T); (11311 12906110 A G); (11312 12906420 T C); (11313 12906571 G
A); (11314 12906751 C T); (11315 12906767 G A); (11316 12906973 G C); (11317 12907176 G A); (11318
12908320 A G); (11319 12908542 A G); (11320 12908652 A T); (11321 12908706 G A); (11322 12908728
C T); (11323 12908999 C T); (11324 12909516 A G); (11325 12909602 T C); (11326 12909682 T C); (11327
12910039 G A); (11328 12910090 T C); (11329 12910127 T C); (11330 12910137 C T); (11331 12910698
G A); (11332 12910808 C T); (11333 12910824 C A); (11334 12911213 T A); (11335 12911408 G
A); (11336 12911954 C T); (11337 12912875 G C); (11338 12912896 G A); (11339 12913064 T A); (11340
12913161 A G); (11341 12914969 T C); (11342 12915008 T C); (11343 12915266 C T); (11344 12915643
A G); (11345 12915645 C T); (11346 12915801 C A); (11347 12915850 A G); (11348 12916593 C
T); (11349 12917943 G A); (11350 12918088 A C); (11351 12918586 T C); (11352 12921356 C CT); (11353
12922601 A G); (11354 12923364 C T); (11355 12924301 A G); (11356 12924315 T C); (11357 12924526
A G); (11358 12924814 G A); (11359 12925240 T C); (11360 12925605 T C); (11361 12926054 G
A); (11362 12926154 AT A); (11363 12926335 T G); (11364 12926694 T A); (11365 12926873 A T); (11366
12927132 T C); (11367 12927177 A G); (11368 12927395 C T); (11369 12929024 A G); (11370 12929552
A G); (11371 12931567 A G); (11372 12932001 T C); (11373 12932117 C T); (11374 12932171 C
T); (11375 12932232 G A); (11376 12932393 A G); (11377 12932778 C A); (11378 12932793 A G); (11379
12932995 T C); (11380 12933015 G A); (11381 12933443 G A); (11382 12933468 C T); (11383 12933573
C A); (11384 12933917 C T); (11385 12934043 T C); (11386 12934210 A G); (11387 12934471 AC
A); (11388 12934561 T C); (11389 12934575 T C); (11390 12935237 C T); (11391 12936202 C G); (11392
12936285 G C); (11393 12936928 A G); (11394 12937471 T TTTTTCTCTATTTTTCTGGG); (11395
12937491 T C); (11396 12937498 T C); (11397 12937510 G A); (11398 12937523 G A); (11399 12937563
T G); (11400 12937634 G A); (11401 12937892 T TGTAGAAGTAGGA); (11402 12937966 C T); (11403
12937988 G A); (11404 12938006 G C); (11405 12938099 G A); (11406 12938771 A G); (11407 12938779
A G); (11408 12939064 G A); (11409 12939158 G A); (11410 12939178 G A); (11411 12939242 G
A); (11412 12940882 T C); (11413 12940915 A T); (11414 12943004 G A); (11415 12943031 T G); (11416
12943594 T C); (11417 12943927 G C); (11418 12944219 A G); (11419 12944353 A G); (11420 12944538
T C); (11421 12944734 T C); (11422 12945284 G A); (11423 12945315 G T); (11424 12945493 C
A); (11425 12947318 C T); (11426 12947352 A T); (11427 12947597 T TATTAGCTTGTAATGA); (11428
12948177 C T); (11429 12948280 C T); (11430 12948302 T C); (11431 12948427 T C); (11432 12948824 C
T); (11433 12949163 C A); (11434 12949265 A T); (11435 12951885 A T); (11436 12951888 G A); (11437
12953359 A T); (11438 12953493 T C); (11439 12953497 A G); (11440 12953620 G A); (11441 12953812
CT C); (11442 12953974 A G); (11443 12954090 C T); (11444 12954207 A C); (11445 12954255 T
A); (11446 12954382 G A); (11447 12955087 T C); (11448 12955460 G A); (11449 12955498 G A); (11450
12955696 C A); (11451 12956042 A AT); (11452 12956060 T G); (11453 12956479 A G); (11454
12956486 G T); (11455 12956800 G A); (11456 12957115 C T); (11457 12957793 C T); (11458 12957931
T C); (11459 12958460 C T); (11460 12958957 A G); (11461 12958965 C T); (11462 12959534 T A); (11463
12959664 T A); (11464 12959679 A G); (11465 12959691 T C); (11466 12959717 T C); (11467 12959724
A G); (11468 12959736 G A); (11469 12959788 T A); (11470 12959822 A G); (11471 12959844 T
C); (11472 12963512 G A); (11473 12963774 C G); (11474 12963894 A G); (11475 12964549 G A); (11476
12965058 G T); (11477 12965139 T C); (11478 12965239 G A); (11479 12965454 C A); (11480 12965501
T A); (11481 12965514 A T); (11482 12965820 T C); (11483 12965938 G A); (11484 12966029 C
A); (11485 12966032 A C); (11486 12966284 G T); (11487 12966663 G A); (11488 12966771 C T); (11489
12967646 G T); (11490 12968696 G A); (11491 12968732 T A); (11492 12968736 T A); (11493 12982696
T A); (11494 12982698 A T); (11495 12982766 CT C); (11496 12983791 C CCAA); (11497 12985627 C
A); (11498 12985761 G A); (11499 12986000 G A); (11500 12986918 T A); (11501 12986919 T A); (11502
12987071 A C); (11503 12987109 AG A); (11504 12987208 G GA); (11505 12987350 A G); (11506
12987974 A C); (11507 12987981 T C); (11508 12990417 C T); (11509 12990443 C T); (11510 12990791 T
C); (11511 12990974 T C); (11512 12991015 C A); (11513 12991112 A G); (11514 12991186 A C); (11515
12991203 A C); (11516 12991277 C T); (11517 12991354 A G); (11518 12991382 G A); (11519 12991540
T C); (11520 12991600 T C); (11521 12991746 G A); (11522 12991835 C T); (11523 12992134 G
A); (11524 12992142 G A); (11525 12992778 G T); (11526 12993205 C T); (11527 12993418 A G); (11528
12993880 G T); (11529 12993918 G A); (11530 12993997 T C); (11531 12994150 C T); (11532 12994240
A G); (11533 12994353 G T); (11534 12994526 GA G); (11535 12995160 C T); (11536 12995543 T
C); (11537 12995840 T C); (11538 12995940 A G); (11539 12996948 A C); (11540 13003721 C A); (11541
13003849 G A); (11542 13003878 C T); (11543 13004120 C G); (11544 13004859 C G); (11545 13004967
C T); (11546 13005147 G A); (11547 13005264 T A); (11548 13005300 G A); (11549 13005303 T
C); (11550 13005304 A T); (11551 13005333 C T); (11552 13005379 C A); (11553 13005526 A C); (11554
13005996 T C); (11555 13006121 T G); (11556 13006295 C T); (11557 13006385 T C); (11558 13006480
G A); (11559 13006673 C T); (11560 13006811 A G); (11561 13006841 C G); (11562 13007376 G
A); (11563 13007458 T C); (11564 13007753 C T); (11565 13008497 C T); (11566 13008520 A
AAAACAGAC); (11567 13008576 C T); (11568 13009334 G C); (11569 13009521 C A); (11570 13009534 G
A); (11571 13009797 A G); (11572 13009865 G A); (11573 13010076 G A); (11574 13010109 C T); (11575
13010131 C T); (11576 13010630 G C); (11577 13010786 C A); (11578 13010788 T C); (11579 13011024
C T); (11580 13011213 C T); (11581 13011242 C T); (11582 13011291 C T); (11583 13011375 G A); (11584
13011596 G A); (11585 13011654 G A); (11586 13011781 G A); (11587 13011799 C T); (11588 13011867
A G); (11589 13012237 C T); (11590 13012308 C T); (11591 13012436 A C); (11592 13012492 G
A); (11593 13012659 T C); (11594 13012776 T C); (11595 13012971 C T); (11596 13013061 T TG); (11597
13013381 C T); (11598 13013490 C T); (11599 13013646 A G); (11600 13013720 T A); (11601 13013753
G T); (11602 13013904 C T); (11603 13014035 A G); (11604 13014373 G T); (11605 13014395 G
A); (11606 13014404 T C); (11607 13014755 A G); (11608 13014815 T C); (11609 13015325 A C); (11610
13015601 C T); (11611 13016142 A G); (11612 13016304 C G); (11613 13016526 C T); (11614 13016635
A G); (11615 13016772 T C); (11616 13016953 C G); (11617 13017319 TATA T); (11618 13017404 A
T); (11619 13017419 A T); (11620 13017745 C T); (11621 13017791 G A); (11622 13017823 A G); (11623
13017898 T C); (11624 13018040 T G); (11625 13018254 A T); (11626 13018574 G A); (11627 13019013
A T); (11628 13019304 G GA); (11629 13020269 A G); (11630 13020926 T TACCCCCC); (11631 13021206
C G); (11632 13021559 C A); (11633 13021560 A T); (11634 13022402 T A); (11635 13022505 C T); (11636
13023139 G A); (11637 13023166 T G); (11638 13024091 A G); (11639 13024093 T G); (11640 13024100
G A); (11641 13024422 T C); (11642 13024694 A G); (11643 13024918 G A); (11644 13025074 G
A); (11645 13025151 T C); (11646 13025710 G T); (11647 13025916 G A); (11648 13026129 A T); (11649
13026931 T C); (11650 13027404 A C); (11651 13028209 GA G); (11652 13030176 TA T); (11653
13032138 GA G); (11654 13032348 C A); (11655 13035078 T G); (11656 13035094 T C); (11657
13035150 T G); (11658 13036599 A T); (11659 13036798 T C); (11660 13036880 C T); (11661 13036906
G A); (11662 13037027 C A); (11663 13037896 T C); (11664 13038290 C G); (11665 13038612 A
G); (11666 13038727 A G); (11667 13039138 T C); (11668 13039301 G A); (11669 13039536 C T); (11670
13039592 C T); (11671 13039997 C A); (11672 13040003 G A); (11673 13040192 C G); (11674 13040214
T C); (11675 13041175 C T); (11676 13041207 A G); (11677 13041240 T C); (11678 13041946 T C); (11679
13042176 A G); (11680 13042483 A G); (11681 13043007 A C); (11682 13043099 C T); (11683 13043387
G A); (11684 13043566 C A); (11685 13043814 T C); (11686 13043942 T A); (11687 13044372 G
C); (11688 13044526 T C); (11689 13044815 GT G); (11690 13045265 G A); (11691 13045394 T G); (11692
13045436 G A); (11693 13045696 G A); (11694 13046512 T C); (11695 13046571 T C); (11696 13046689
A G); (11697 13047782 G A); (11698 13047870 C G); (11699 13048870 A G); (11700 13048872 C
T); (11701 13048973 C T); (11702 13049018 G A); (11703 13049175 A G); (11704 13049423 G A); (11705
13049565 C A); (11706 13049656 A G); (11707 13049710 CAACA C); (11708 13050709 G A); (11709
13053904 G A); (11710 13065328 T C); (11711 13065386 A G); (11712 13065676 T A); (11713 13066244
A G); (11714 13068041 C T); (11715 13068113 C T); (11716 13068357 A G); (11717 13068439 C
T); (11718 13068576 C T); (11719 13068670 A G); (11720 13068862 T C); (11721 13068908 A G); (11722
13068911 T C); (11723 13069058 G GT); (11724 13070098 T C); (11725 13070504 G A); (11726 13070696
A G); (11727 13070724 A C); (11728 13070958 A G); (11729 13071144 A G); (11730 13071384 C
CAT); (11731 13071401 G A); (11732 13071417 G A); (11733 13071934 T A); (11734 13073483 C
T); (11735 13074172 A G); (11736 13074822 A G); (11737 13075580 T A); (11738 13076811 T C); (11739
13076858 G A); (11740 13076924 A G); (11741 13076977 A G); (11742 13077174 C T); (11743 13077610
G A); (11744 13077718 T TTGAACAATATGACAA); (11745 13077766 A G); (11746 13078348 T
TAGTTAAAA); (11747 13078537 A AC); (11748 13079277 A G); (11749 13079394 A G); (11750 13079413
C T); (11751 13079417 A G); (11752 13079949 T TG); (11753 13080788 C T); (11754 13080808 G
A); (11755 13080829 C T); (11756 13081032 C T); (11757 13081309 C T); (11758 13081389 A G); (11759
13081416 G A); (11760 13081473 T C); (11761 13081826 T A); (11762 13082052 C T); (11763 13082281
G A); (11764 13083037 C T); (11765 13083161 A T); (11766 13083162 A T); (11767 13083466 AC
A); (11768 13083524 C T); (11769 13084341 A C); (11770 13084377 C T); (11771 13084409 C T); (11772
13084695 C T); (11773 13085009 G A); (11774 13085307 C T); (11775 13085543 A G); (11776 13085544
G A); (11777 13085788 T C); (11778 13085892 G A); (11779 13086678 A G); (11780 13086739 G
A); (11781 13086746 A G); (11782 13086747 A G); (11783 13086924 C T); (11784 13086945 G A); (11785
13087037 A C); (11786 13087063 G T); (11787 13087626 A C); (11788 13087988 G A); (11789 13088163
T G); (11790 13088491 A T); (11791 13089986 G A); (11792 13090190 G C); (11793 13090306 C
T); (11794 13090525 T G); (11795 13091003 A G); (11796 13091101 G A); (11797 13091566 C A); (11798
13092018 G A); (11799 13092945 A AT); (11800 13092974 A AGGG); (11801 13093355 T C); (11802
13093413 G A); (11803 13093631 G A); (11804 13093670 C T); (11805 13093677 T G); (11806 13093724
C T); (11807 13093765 A G); (11808 13094742 T C); (11809 13094801 C T); (11810 13094834 G
A); (11811 13094871 C T); (11812 13094908 A G); (11813 13094915 C A); (11814 13095057 A G); (11815
13095061 T C); (11816 13096032 GTATGCAAATA G); (11817 13096096 A G); (11818 13096130 C
T); (11819 13096780 T A); (11820 13096786 G A); (11821 13098920 T C); (11822 13098969 C T); (11823
13098988 T G); (11824 13099008 C T); (11825 13100328 C T); (11826 13101200 G A); (11827 13101206
G A); (11828 13101207 A G); (11829 13104034 A G); (11830 13104920 A G); (11831 13105427 A
G); (11832 13105462 G T); (11833 13106299 G A); (11834 13106331 C T); (11835 13106419 C T); (11836
13106700 A G); (11837 13106784 G T); (11838 13106899 C T); (11839 13106912 G A); (11840 13107003
A G); (11841 13107090 G A); (11842 13107264 G A); (11843 13107282 A AG); (11844 13107717 T
C); (11845 13107825 C A); (11846 13107961 T G); (11847 13108793 C T); (11848 13108860 TA T); (11849
13108959 G C); (11850 13109327 C A); (11851 13109376 T G); (11852 13109500 C A); (11853 13109597
A G); (11854 13109640 G T); (11855 13110010 T A); (11856 13110047 G A); (11857 13110553 C
T); (11858 13110570 C T); (11859 13110592 GA G); (11860 13110838 C G); (11861 13111156 C T); (11862
13111510 T C); (11863 13111830 C T); (11864 13111985 A G); (11865 13113572 C T); (11866 13115165
A G); (11867 13115230 G T); (11868 13115321 T C); (11869 13115496 G A); (11870 13115966 G
A); (11871 13116988 G A); (11872 13117081 A G); (11873 13117085 G A); (11874 13117498 TG
T); (11875 13117605 A G); (11876 13117846 C T); (11877 13117874 A G); (11878 13117878 T C); (11879
13117918 A G); (11880 13117957 C T); (11881 13117960 A G); (11882 13117967 T G); (11883 13118018
G A); (11884 13118055 G A); (11885 13120335 A G); (11886 13120680 A G); (11887 13121130 A
G); (11888 13121193 A G); (11889 13121340 A G); (11890 13122235 T C); (11891 13123492 G A); (11892
13123502 G A); (11893 13123529 T C); (11894 13124006 G A); (11895 13124877 G A); (11896 13124924
G A); (11897 13125881 G T); (11898 13126229 T C); (11899 13126249 G A); (11900 13126618 G
A); (11901 13126863 GC G); (11902 13126931 G A); (11903 13127083 G C); (11904 13127976 A
C); (11905 13128562 C A); (11906 13128700 A G); (11907 13128722 G A); (11908 13131558 A G); (11909
13135008 C A); (11910 13135012 T A); (11911 13135047 C A); (11912 13135053 T C); (11913 13135190
G A); (11914 13135446 C T); (11915 13135572 C A); (11916 13135792 A G); (11917 13135832 A
G); (11918 13135861 T C); (11919 13136247 C T); (11920 13136397 A G); (11921 13136485 G A); (11922
13136500 G A); (11923 13136827 C A); (11924 13136984 G C); (11925 13137151 C T); (11926 13137174
T C); (11927 13137233 G T); (11928 13137295 C T); (11929 13137614 A G); (11930 13137885 A T); (11931
13138043 A T); (11932 13138103 G C); (11933 13138121 C T); (11934 13138135 T C); (11935 13138158 C
T); (11936 13138479 G A); (11937 13138571 G A); (11938 13138784 A G); (11939 13139227 C T); (11940
13139466 G T); (11941 13139559 C T); (11942 13141849 T TAA); (11943 13142011 T C); (11944
13142348 T C); (11945 13142405 T C); (11946 13142901 A AATT); (11947 13143514 C CATGATT); (11948
13143697 T A); (11949 13144145 A G); (11950 13144154 A C); (11951 13144371 C T); (11952 13144426
C T); (11953 13144700 T G); (11954 13145878 C T); (11955 13145977 A C); (11956 13146086 G
A); (11957 13146163 T C); (11958 13146164 T C); (11959 13146417 G C); (11960 13146672 T C); (11961
13146828 C T); (11962 13149580 C T); (11963 13149869 C T); (11964 13150006 T C); (11965 13150066 T
C); (11966 13150089 G T); (11967 13150462 G A); (11968 13150531 C A); (11969 13150575 T C); (11970
13150835 A G); (11971 13150891 T C); (11972 13150906 T A); (11973 13150957 T C); (11974 13151001
G A); (11975 13151191 A G); (11976 13151969 G T); (11977 13152081 G C); (11978 13153116 A
AT); (11979 13153754 T C); (11980 13153931 G T); (11981 13154181 T C); (11982 13154348 T C); (11983
13154898 C A); (11984 13155088 A ATCTTCTTCCTTAAACACTATTGTGT); (11985 13155558 C T); (11986
13156470 C T); (11987 13156514 G A); (11988 13157415 A C); (11989 13157427 C A); (11990 13157447
T C); (11991 13157500 A G); (11992 13157671 C T); (11993 13158950 T G); (11994 13159430 G
T); (11995 13159784 G A); (11996 13160369 T A); (11997 13160377 G A); (11998 13160519 G C); (11999
13160534 C T); (12000 13160553 T G); (12001 13161110 C A); (12002 13161157 A C); (12003 13162163
T G); (12004 13162202 A G); (12005 13162515 T C); (12006 13162779 GT G); (12007 13162887 T
A); (12008 13162963 G C); (12009 13163209 G A); (12010 13163213 CTG C); (12011 13163362 AT
A); (12012 13163379 A T); (12013 13163400 C G); (12014 13163402 T C); (12015 13163410 T C); (12016
13163560 G A); (12017 13163564 A G); (12018 13163967 A T); (12019 13163974 C T); (12020 13164376
C T); (12021 13164563 G A); (12022 13164946 T C); (12023 13165151 T C); (12024 13165252 A
G); (12025 13165376 C T); (12026 13165685 G A); (12027 13166343 C G); (12028 13166394 A C); (12029
13166795 A G); (12030 13167055 CT C); (12031 13167258 A G); (12032 13167317 T A); (12033
13167597 A AT); (12034 13167807 C T); (12035 13167836 G A); (12036 13167975 G T); (12037 13168375
A C); (12038 13168656 G A); (12039 13169170 C T); (12040 13169439 G GT); (12041 13169497 C
G); (12042 13169509 T C); (12043 13169920 G T); (12044 13170076 A G); (12045 13170330 C G); (12046
13170437 A C); (12047 13170859 C T); (12048 13171212 T C); (12049 13171445 C G); (12050 13171677
G T); (12051 13171847 A AT); (12052 13172843 T C); (12053 13172884 G T); (12054 13173255 A
T); (12055 13174471 G C); (12056 13174477 A T); (12057 13175076 C A); (12058 13175547 TA T); (12059
13177672 T C); (12060 13179029 T G); (12061 13179148 G A); (12062 13179254 C T); (12063 13179453
C G); (12064 13179474 T C); (12065 13179489 T G); (12066 13179595 C T); (12067 13179747 C T); (12068
13179779 T C); (12069 13180106 C G); (12070 13180345 G A); (12071 13180460 C T); (12072 13180707
G A); (12073 13181487 A C); (12074 13181623 A G); (12075 13181718 A G); (12076 13182264 C
T); (12077 13182317 G A); (12078 13182521 T C); (12079 13182812 T C); (12080 13182983 G A); (12081
13183063 T C); (12082 13183118 AG A); (12083 13183612 G A); (12084 13183892 C A); (12085
13184248 A G); (12086 13185609 C T); (12087 13185767 A G); (12088 13186071 C T); (12089 13186438
C T); (12090 13186610 A G); (12091 13187586 A C); (12092 13188291 T A); (12093 13188432 T C); (12094
13194288 C T); (12095 13196213 G A); (12096 13198101 C T); (12097 13198162 T A); (12098 13198271
G A); (12099 13199289 G A); (12100 13199349 C T); (12101 13199487 G A); (12102 13199700 T
C); (12103 13199802 C T); (12104 13199892 A G); (12105 13199936 C T); (12106 13199937 G A); (12107
13199943 A C); (12108 13200017 C T); (12109 13200026 C T); (12110 13203031 C T); (12111 13203889 G
A); (12112 13203905 A G); (12113 13203986 T C); (12114 13204069 AG A); (12115 13204101 C T); (12116
13204274 T C); (12117 13205116 G C); (12118 13205727 C T); (12119 13205942 G A); (12120 13206060
CATTAT C); (12121 13206122 A G); (12122 13206309 T C); (12123 13206624 T C); (12124 13207665 A
G); (12125 13208346 G A); (12126 13208408 A G); (12127 13208409 A G); (12128 13208641 T C); (12129
13209045 A G); (12130 13209452 G A); (12131 13210088 C T); (12132 13210102 C G); (12133 13210106
A T); (12134 13210142 A G); (12135 13210375 A G); (12136 13211939 T G); (12137 13211994 G
A); (12138 13212039 G C); (12139 13212123 T C); (12140 13212309 G A); (12141 13212322 G A); (12142
13212603 G A); (12143 13212789 C T); (12144 13212885 A C); (12145 13212913 T G); (12146 13213087
T C); (12147 13214495 G A); (12148 13214614 T C); (12149 13214660 G A); (12150 13214984 G
A); (12151 13215772 G A); (12152 13215840 G T); (12153 13216232 C A); (12154 13216844 C T); (12155
13217393 C T); (12156 13217405 T A); (12157 13217655 A T); (12158 13217733 A G); (12159 13217835
A G); (12160 13217847 T C); (12161 13217865 C T); (12162 13217926 T C); (12163 13218287 T G); (12164
13218377 C A); (12165 13218439 G A); (12166 13218593 G C); (12167 13218625 A T); (12168 13218632
A G); (12169 13218763 T C); (12170 13218768 C G); (12171 13218948 C T); (12172 13219361 T
G); (12173 13219381 T C); (12174 13219787 T G); (12175 13219925 G A); (12176 13220032 T C); (12177
13220041 A G); (12178 13221364 G C); (12179 13221473 G A); (12180 13221642 C T); (12181 13221732
C T); (12182 13222183 C T); (12183 13222202 G A); (12184 13223186 C T); (12185 13223276 A
G); (12186 13223437 C T); (12187 13223669 T C); (12188 13223776 A G); (12189 13223837 G A); (12190
13224272 A G); (12191 13224324 G A); (12192 13224359 G A); (12193 13224434 G A); (12194 13224455
T C); (12195 13224527 C G); (12196 13224557 G A); (12197 13224636 T C); (12198 13225024 T C); (12199
13225108 C A); (12200 13225118 T C); (12201 13225212 T C); (12202 13225288 C T); (12203 13225368 T
C); (12204 13225746 C G); (12205 13225981 A G); (12206 13226129 C T); (12207 13226273 T G); (12208
13226366 G A); (12209 13226440 T C); (12210 13226720 CT C); (12211 13226813 G A); (12212 13226896
A G); (12213 13227035 A G); (12214 13227480 C T); (12215 13227717 G A); (12216 13228636 A
G); (12217 13228686 T A); (12218 13229928 G A); (12219 13229938 C T); (12220 13229986 A T); (12221
13231130 C T); (12222 13231367 A G); (12223 13231574 A G); (12224 13231598 C T); (12225 13231756
C G); (12226 13231847 A G); (12227 13232001 T C); (12228 13232456 C T); (12229 13232736 G
T); (12230 13232763 G A); (12231 13232784 C G); (12232 13233139 C T); (12233 13233372 C T); (12234
13233556 A C); (12235 13233721 A AATAT); (12236 13234565 T C); (12237 13234810 T C); (12238
13234824 G A); (12239 13235372 C T); (12240 13235952 C T); (12241 13236410 A G); (12242 13236555
T G); (12243 13236643 G A); (12244 13236681 A G); (12245 13236737 G A); (12246 13236979 G
A); (12247 13238708 A G); (12248 13238797 AAAGT A); (12249 13239102 T C); (12250 13239105 C
T); (12251 13239644 A G); (12252 13240302 T G); (12253 13240633 A C); (12254 13241293 A G); (12255
13242270 TTG T); (12256 13242727 C T); (12257 13242938 GA G); (12258 13243272 T C); (12259
13243712 G A); (12260 13243887 A G); (12261 13244108 C T); (12262 13244268 C T); (12263 13244395
G C); (12264 13244559 C T); (12265 13244596 G A); (12266 13244648 A G); (12267 13244947 A
G); (12268 13244958 C T); (12269 13245119 A G); (12270 13245135 G A); (12271 13245251 G C); (12272
13245288 G A); (12273 13246049 A G); (12274 13246121 TG T); (12275 13246467 A G); (12276
13247559 A G); (12277 13247588 G A); (12278 13248005 C T); (12279 13248657 G A); (12280 13249664
C T); (12281 13249728 G A); (12282 13249914 G A); (12283 13250015 C T); (12284 13250027 C
T); (12285 13250823 A G); (12286 13250839 C T); (12287 13251164 T C); (12288 13251364 T C); (12289
13251390 T C); (12290 13251523 G T); (12291 13251814 A G); (12292 13251880 T C); (12293 13251919
T C); (12294 13252760 A C); (12295 13252811 A G); (12296 13252860 A G); (12297 13252935 G
C); (12298 13253158 T C); (12299 13253295 G A); (12300 13253441 T C); (12301 13253771 A G); (12302
13253797 G T); (12303 13253798 T A); (12304 13255146 T G); (12305 13255177 G A); (12306 13255607
T C); (12307 13255787 G A); (12308 13256182 T C); (12309 13256498 A C); (12310 13256745 G
A); (12311 13256747 A C); (12312 13257373 C A); (12313 13257597 A G); (12314 13257905 G A); (12315
13258071 C T); (12316 13258669 T C); (12317 13258782 T C); (12318 13258826 GT G); (12319 13259435
T C); (12320 13259825 C G); (12321 13259965 A T); (12322 13260775 GT G); (12323 13260874 C
T); (12324 13261089 G GAGA); (12325 13261213 T TATGTGGCAGATGCAAGTG); (12326 13261424 A
G); (12327 13261686 G A); (12328 13262217 C T); (12329 13262454 G T); (12330 13263517 C T); (12331
13264078 T A); (12332 13264110 GA G); (12333 13264403 C A); (12334 13264519 A G); (12335
13264791 A G); (12336 13264834 TC T); (12337 13265174 A C); (12338 13265823 C T); (12339 13266160
CT C); (12340 13266275 G T); (12341 13266732 A G); (12342 13266963 C T); (12343 13266974 C
T); (12344 13267138 T A); (12345 13267460 G A); (12346 13267616 T C); (12347 13268121 A G); (12348
13268188 G T); (12349 13268346 T G); (12350 13268410 T TGA); (12351 13268536 C A); (12352
13269033 G T); (12353 13269083 C A); (12354 13270208 A G); (12355 13270606 G A); (12356 13270635
C T); (12357 13270678 G A); (12358 13271229 C T); (12359 13271416 A G); (12360 13271449 A
T); (12361 13271946 T TA); (12362 13272075 A C); (12363 13272191 G T); (12364 13272227 T C); (12365
13272381 T C); (12366 13272529 G A); (12367 13272846 C G); (12368 13273175 A G); (12369 13273346
A T); (12370 13273371 T C); (12371 13273403 C T); (12372 13273710 C A); (12373 13273812 G C); (12374
13274036 C G); (12375 13274292 A T); (12376 13274300 T C); (12377 13274529 G A); (12378 13274632
T C); (12379 13274648 T C); (12380 13274845 G T); (12381 13274962 T C); (12382 13275060 G A); (12383
13275062 A G); (12384 13276011 C CA); (12385 13276175 T C); (12386 13276650 C T); (12387 13277508
T A); (12388 13277509 A C); (12389 13278278 G A); (12390 13278518 T C); (12391 13278837 T C); (12392
13278970 G A); (12393 13279245 C A); (12394 13279318 C T); (12395 13280072 C T); (12396 13280490
AT A); (12397 13280677 A T); (12398 13280730 T A); (12399 13280735 C T); (12400 13281014 T
C); (12401 13281056 C T); (12402 13281465 C G); (12403 13281547 A G); (12404 13281597 G C); (12405
13281647 G A); (12406 13281950 G A); (12407 13282019 T C); (12408 13282142 G T); (12409 13282188
A G); (12410 13282332 C A); (12411 13282502 A C); (12412 13283454 T C); (12413 13284609 A
ACCTAAAACTAG); (12414 13284868 T C); (12415 13284870 C T); (12416 13284879 C A); (12417
13284892 G A); (12418 13284899 G A); (12419 13285211 C T); (12420 13285261 T C); (12421 13285275
C T); (12422 13285488 C G); (12423 13285763 A C); (12424 13285855 T C); (12425 13287698 C T); (12426
13287705 G A); (12427 13287826 G A); (12428 13287866 G A); (12429 13290091 T A); (12430 13290131
C G); (12431 13290301 C T); (12432 13290553 A AT); (12433 13290682 A G); (12434 13291841 T
C); (12435 13292471 G A); (12436 13292503 A G); (12437 13292521 G A); (12438 13292753 T C); (12439
13292843 A C); (12440 13292916 T TA); (12441 13293197 C A); (12442 13293402 C T); (12443 13293513
T C); (12444 13293594 C T); (12445 13294168 C T); (12446 13296297 C T); (12447 13296977 C T); (12448
13297301 G A); (12449 13297332 G A); (12450 13298307 A G); (12451 13298319 A G); (12452 13298356
C T); (12453 13298371 A G); (12454 13298373 T C); (12455 13298376 G A); (12456 13298394 T
A); (12457 13298409 C T); (12458 13298798 T C); (12459 13298802 G C); (12460 13300391 A C); (12461
13300394 C T); (12462 13300892 T C); (12463 13300899 G T); (12464 13300974 C T); (12465 13301883 C
T); (12466 13302159 A G); (12467 13302442 C T); (12468 13302620 T C); (12469 13302659 T C); (12470
13302785 G T); (12471 13302809 C A); (12472 13302858 G A); (12473 13302919 A T); (12474 13302952
C A); (12475 13303001 G A); (12476 13303021 C G); (12477 13303072 C T); (12478 13303092 A
G); (12479 13303141 A C); (12480 13303262 A T); (12481 13304574 A G); (12482 13304583 C T); (12483
13304944 T G); (12484 13304949 C A); (12485 13305153 A AT); (12486 13305198 G A); (12487
13305204 A T); (12488 13305232 T C); (12489 13305473 C T); (12490 13305596 G GT); (12491 13305610
C G); (12492 13306247 C T); (12493 13306290 T C); (12494 13306722 A AT); (12495 13307396 G
T); (12496 13308000 G A); (12497 13308081 T C); (12498 13308977 A T); (12499 13308979 G T); (12500
13310188 A G); (12501 13310229 G A); (12502 13310350 T C); (12503 13310631 G A); (12504 13310641
A T); (12505 13310865 A G); (12506 13311204 C T); (12507 13311665 G C); (12508 13311707 T
C); (12509 13311762 A AT); (12510 13311881 G A); (12511 13312073 G A); (12512 13312183 C T); (12513
13312239 G A); (12514 13312240 A T); (12515 13312241 T C); (12516 13312311 T C); (12517 13312404
C T); (12518 13312604 A G); (12519 13313637 C A); (12520 13314158 T C); (12521 13314210 C T); (12522
13314496 T C); (12523 13314723 G T); (12524 13315702 G A); (12525 13315887 G A); (12526 13315924
G C); (12527 13316519 T C); (12528 13316567 T C); (12529 13316571 T C); (12530 13316591 G T); (12531
13317298 A G); (12532 13317300 C T); (12533 13317515 A G); (12534 13317606 G A); (12535 13317771
G T); (12536 13317817 C T); (12537 13317944 T C); (12538 13317975 G A); (12539 13318160 G
C); (12540 13318173 T C); (12541 13319211 G T); (12542 13319389 A ACATGTGCGAAGCAT); (12543
13319712 G A); (12544 13319884 G GA); (12545 13319973 C T); (12546 13320048 C A); (12547
13320098 T C); (12548 13320275 G T); (12549 13320414 T C); (12550 13320529 G A); (12551 13320530
G C); (12552 13320621 C T); (12553 13321527 T C); (12554 13321592 C T); (12555 13321620 A G); (12556
13322098 G A); (12557 13322162 C T); (12558 13322268 G A); (12559 13322473 A G); (12560 13322635
G A); (12561 13322728 T C); (12562 13322798 TTGGTCAACCATC T); (12563 13323124 C T); (12564
13323365 G A); (12565 13323939 C A); (12566 13323979 C T); (12567 13324054 C T); (12568 13325195
C T); (12569 13325551 T C); (12570 13326045 C T); (12571 13327277 C A); (12572 13327594 T C); (12573
13327905 T C); (12574 13328092 T C); (12575 13328294 G A); (12576 13328446 C T); (12577 13328464 C
T); (12578 13328670 T C); (12579 13329050 T C); (12580 13329140 C G); (12581 13329793 C G); (12582
13329970 C T); (12583 13330065 T A); (12584 13330399 G A); (12585 13330567 C T); (12586 13330758
C T); (12587 13330826 A G); (12588 13331059 T TA); (12589 13331141 T G); (12590 13331503 A
G); (12591 13331631 G A); (12592 13331717 T G); (12593 13331727 C T); (12594 13332025 A G); (12595
13332820 AT A); (12596 13332901 G A); (12597 13333346 AG A); (12598 13333490 A C); (12599
13334390 T TA); (12600 13335297 C G); (12601 13337322 T A); (12602 13337480 T C); (12603 13338064
G A); (12604 13339673 A AT); (12605 13340205 G T); (12606 13340288 T G); (12607 13340810 T
A); (12608 13340847 T A); (12609 13341678 A G); (12610 13341704 G C); (12611 13342265 G A); (12612
13342438 A C); (12613 13342509 A C); (12614 13343062 A G); (12615 13343639 T C); (12616 13344260
G A); (12617 13344811 A G); (12618 13344842 A G); (12619 13345502 C T); (12620 13345506 G
A); (12621 13345552 T C); (12622 13346084 T C); (12623 13346100 G T); (12624 13346127 G A); (12625
13346128 T C); (12626 13346172 T A); (12627 13346348 C CA); (12628 13347047 G T); (12629 13347373
G GT); (12630 13347927 C T); (12631 13348243 T C); (12632 13348345 C T); (12633 13348451 T
C); (12634 13348539 C T); (12635 13348673 T C); (12636 13348740 T TA); (12637 13349060 C T); (12638
13349281 C T); (12639 13349532 C T); (12640 13349608 A G); (12641 13349654 C T); (12642 13349670 C
T); (12643 13349696 C T); (12644 13349851 A G); (12645 13350045 T C); (12646 13350100 A C); (12647
13350280 A G); (12648 13350776 GT G); (12649 13350972 C T); (12650 13351036 T C); (12651 13351260
A T); (12652 13351398 C T); (12653 13351477 AAGT A); (12654 13351478 C A); (12655 13351479 A
C); (12656 13354260 C T); (12657 13355668 T C); (12658 13356241 G T); (12659 13356250 C T); (12660
13356286 A G); (12661 13356304 G T); (12662 13356695 G C); (12663 13356834 C T); (12664 13356938
T C); (12665 13357177 C T); (12666 13357363 C T); (12667 13357370 C T); (12668 13357627 A T); (12669
13357774 A G); (12670 13358070 T C); (12671 13358075 G A); (12672 13358093 C T); (12673 13358199
C T); (12674 13358223 C T); (12675 13358322 C T); (12676 13358768 C T); (12677 13358769 T C); (12678
13358770 A C); (12679 13359036 T C); (12680 13359037 C T); (12681 13359044 C T); (12682 13359109 T
G); (12683 13359127 T A); (12684 13359281 G A); (12685 13359738 G A); (12686 13360001 C T); (12687
13360136 T C); (12688 13360304 G C); (12689 13360620 A G); (12690 13361525 T A); (12691 13361741
C A); (12692 13361742 T A); (12693 13362174 A G); (12694 13362459 C T); (12695 13362614 A
G); (12696 13363160 G GTA); (12697 13363242 G A); (12698 13363430 G A); (12699 13363607 T
A); (12700 13363825 C T); (12701 13365143 T A); (12702 13365356 C T); (12703 13365499 A G); (12704
13365957 T C); (12705 13366186 G A); (12706 13366358 A T); (12707 13367253 G GA); (12708
13368253 C G); (12709 13368291 G A); (12710 13368370 G A); (12711 13368437 C T); (12712 13368519
G A); (12713 13368530 A G); (12714 13368791 G GA); (12715 13368904 G A); (12716 13369065 A
G); (12717 13369286 G A); (12718 13369525 T C); (12719 13370371 T C); (12720 13370621 G T); (12721
13370681 A AT); (12722 13370827 TA T); (12723 13371485 T C); (12724 13371707 A G); (12725
13371717 T C); (12726 13371981 A G); (12727 13373353 A G); (12728 13373472 T C); (12729 13373626
G T); (12730 13373869 G A); (12731 13374738 A T); (12732 13374977 G A); (12733 13375018 C
T); (12734 13375690 A G); (12735 13375729 C A); (12736 13375830 A G); (12737 13376822 C T); (12738
13378089 A C); (12739 13378718 G C); (12740 13378932 C T); (12741 13380154 C T); (12742 13380244
G A); (12743 13380602 T C); (12744 13380717 C T); (12745 13380752 A T); (12746 13381034 T C); (12747
13381245 T G); (12748 13381787 C T); (12749 13381965 T C); (12750 13383029 A G); (12751 13383094
T C); (12752 13383362 T A); (12753 13384312 C T); (12754 13384625 C G); (12755 13384986 A T); (12756
13385044 C T); (12757 13385427 T C); (12758 13385492 G A); (12759 13385837 C T); (12760 13386663
G A); (12761 13386964 C G); (12762 13387558 T C); (12763 13387590 G T); (12764 13388089 G
A); (12765 13388112 G A); (12766 13388161 T G); (12767 13388796 C A); (12768 13389150 C A); (12769
13389785 A C); (12770 13389876 A C); (12771 13390162 C T); (12772 13390485 T C); (12773 13390654 T
C); (12774 13390671 A G); (12775 13390938 G A); (12776 13391549 T A); (12777 13391906 A G); (12778
13392093 G A); (12779 13392295 A C); (12780 13392403 T C); (12781 13393302 G A); (12782 13393352
G A); (12783 13393375 T C); (12784 13393435 A G); (12785 13394508 C T); (12786 13394791 G
T); (12787 13394792 T A); (12788 13395027 G A); (12789 13395403 GT G); (12790 13395871 T C); (12791
13396077 CT C); (12792 13396471 A C); (12793 13396636 T A); (12794 13396676 CAT C); (12795
13396723 A G); (12796 13396809 A G); (12797 13396980 G A); (12798 13397310 C T); (12799 13397420
G A); (12800 13397437 T C); (12801 13397506 A G); (12802 13397589 G C); (12803 13397591 A
T); (12804 13397772 A G); (12805 13397890 T A); (12806 13398795 C T); (12807 13398859 C T); (12808
13398989 C T); (12809 13399109 A G); (12810 13399119 A G); (12811 13399140 C T); (12812 13399179
A C); (12813 13399277 C T); (12814 13399286 T C); (12815 13399927 C T); (12816 13399958 C T); (12817
13400480 G A); (12818 13401305 G A); (12819 13401430 C T); (12820 13401788 G C); (12821 13401797
G C); (12822 13401812 C T); (12823 13401836 T G); (12824 13402126 T C); (12825 13402145 ATTGTT
A); (12826 13402178 C T); (12827 13403643 A AG); (12828 13403997 CT C); (12829 13404885 T
A); (12830 13406078 G A); (12831 13406592 G C); (12832 13406806 T A); (12833 13407005 T G); (12834
13407290 C T); (12835 13407675 G C); (12836 13408395 T C); (12837 13409293 T C); (12838 13409727 A
T); (12839 13409999 G A); (12840 13410053 G A); (12841 13410080 G A); (12842 13410088 A G); (12843
13410110 A AT); (12844 13410236 A AG); (12845 13410323 A G); (12846 13411641 A G); (12847
13411882 C G); (12848 13412028 C T); (12849 13412319 CGATTT C); (12850 13412686 A G); (12851
13412809 T C); (12852 13413126 T C); (12853 13413545 G A); (12854 13413601 T C); (12855 13414357 T
A); (12856 13414358 T A); (12857 13414471 A G); (12858 13414608 A C); (12859 13414619 C T); (12860
13414693 A G); (12861 13414753 C T); (12862 13414762 T C); (12863 13415263 T G); (12864 13416421
T C); (12865 13416501 TC T); (12866 13416865 C G); (12867 13417275 TC T); (12868 13417917 A
ACAGG); (12869 13418100 C A); (12870 13418117 G A); (12871 13418414 TTTG T); (12872 13418945 GA
G); (12873 13419574 C T); (12874 13419723 C T); (12875 13419813 C T); (12876 13419849 A G); (12877
13419861 T TA); (12878 13419941 A T); (12879 13420163 AT A); (12880 13420307 C A); (12881
13420375 A AT); (12882 13420449 T C); (12883 13420518 T TTA); (12884 13421157 C T); (12885
13421465 T C); (12886 13421968 A G); (12887 13422590 C T); (12888 13423195 A G); (12889 13423289
T G); (12890 13423295 C T); (12891 13423779 A C); (12892 13423907 G A); (12893 13423962 G
A); (12894 13423974 G A); (12895 13425209 G A); (12896 13425678 T C); (12897 13425713 T TA); (12898
13426315 T C); (12899 13426750 G C); (12900 13426812 T C); (12901 13426885 A G); (12902 13426908
C T); (12903 13426936 T C); (12904 13427630 T C); (12905 13428524 T A); (12906 13428678 C A); (12907
13429960 T C); (12908 13430030 G A); (12909 13430777 A G); (12910 13431256 C T); (12911 13431463
TC T); (12912 13431494 A G); (12913 13431522 G A); (12914 13432114 G T); (12915 13432183 CT
C); (12916 13432222 T G); (12917 13432315 A C); (12918 13432356 G A); (12919 13433720 C T); (12920
13434907 C A); (12921 13434958 TTATATATATATATATATATATA T); (12922 13435014 G A); (12923
13435151 T C); (12924 13435539 A G); (12925 13435651 G A); (12926 13435658 T C); (12927 13435728
A G); (12928 13436102 A G); (12929 13437038 C A); (12930 13438343 A G); (12931 13439085 G
A); (12932 13439130 G T); (12933 13440047 C G); (12934 13440301 A C); (12935 13441669 A C); (12936
13441681 A G); (12937 13441773 T G); (12938 13442247 T G); (12939 13442863 T A); (12940 13443782
AC A); (12941 13443861 A G); (12942 13444235 C T); (12943 13445264 A T); (12944 13445328 C
T); (12945 13445374 C T); (12946 13445432 T C); (12947 13445468 T C); (12948 13445650 G A); (12949
13446226 T A); (12950 13446651 T C); (12951 13447184 T C); (12952 13447256 T C); (12953 13447305 G
A); (12954 13447638 T A); (12955 13447755 G A); (12956 13447894 G A); (12957 13448923 A G); (12958
13449684 T C); (12959 13449907 A G); (12960 13451370 C A); (12961 13451804 G C); (12962 13452098
T C); (12963 13452721 T G); (12964 13453012 A G); (12965 13453228 C T); (12966 13453546 A T); (12967
13453793 C T); (12968 13454384 A C); (12969 13454620 A G); (12970 13454860 G T); (12971 13454941
AAAAGTTT A); (12972 13456558 C T); (12973 13457017 A G); (12974 13457136 C T); (12975 13457938 A
G); (12976 13458033 AC A); (12977 13458247 C T); (12978 13458308 T C); (12979 13458473 A G); (12980
13458751 G A); (12981 13459257 G A); (12982 13459294 A G); (12983 13459317 G A); (12984 13459510
T C); (12985 13459555 C T); (12986 13459591 A C); (12987 13459595 C T); (12988 13459655 G A); (12989
13460264 T G); (12990 13460301 T TG); (12991 13460667 G T); (12992 13460694 A T); (12993 13462280
GT G); (12994 13462780 T TA); (12995 13462979 A T); (12996 13463233 A G); (12997 13463965 C
T); (12998 13463966 A T); (12999 13465167 TA T); (13000 13465269 T A); (13001 13465324 T C); (13002
13465415 G C); (13003 13466193 G A); (13004 13466255 A G); (13005 13466398 C T); (13006 13466698
C T); (13007 13467152 C T); (13008 13467240 T A); (13009 13467503 AT A); (13010 13467813 T
C); (13011 13467815 T C); (13012 13467819 T C); (13013 13467821 T C); (13014 13467823 T C); (13015
13468145 A T); (13016 13468164 C T); (13017 13468303 T C); (13018 13468405 A T); (13019 13468844 C
A); (13020 13469208 A G); (13021 13469233 A C); (13022 13469234 C T); (13023 13469275 C T); (13024
13469291 A C); (13025 13469332 C T); (13026 13469499 T A); (13027 13469799 G C); (13028 13470014
C T); (13029 13470131 A C); (13030 13470164 TA T); (13031 13470307 T C); (13032 13470447 A
AT); (13033 13470571 C G); (13034 13471761 T C); (13035 13471784 TAATTG T); (13036 13471789 C
T); (13037 13471858 C CT); (13038 13471881 G C); (13039 13471908 C T); (13040 13472060 TTTG
T); (13041 13472354 G T); (13042 13472429 C G); (13043 13473183 C T); (13044 13474077 T TA); (13045
13474261 A G); (13046 13475325 T C); (13047 13475411 C A); (13048 13475462 A G); (13049 13475647
C T); (13050 13475727 C A); (13051 13475865 A G); (13052 13475866 A G); (13053 13476276 T
C); (13054 13476570 C A); (13055 13476619 T C); (13056 13476700 AT A); (13057 13476784 G A); (13058
13477144 T C); (13059 13477400 G C); (13060 13477419 A G); (13061 13477503 T TTATATATA); (13062
13477988 G A); (13063 13478217 A G); (13064 13479072 CG C); (13065 13479141 C T); (13066
13479557 A G); (13067 13480682 T C); (13068 13480795 C T); (13069 13480897 A G); (13070 13481634
A AAT); (13071 13481710 A G); (13072 13481961 G GT); (13073 13482632 TCATATTTAAATC T); (13074
13482643 T C); (13075 13483414 T G); (13076 13483685 A C); (13077 13483846 A G); (13078 13484597
T C); (13079 13484598 T C); (13080 13484629 G A); (13081 13485367 C T); (13082 13485513 A
G); (13083 13485631 C T); (13084 13485728 G A); (13085 13485827 T C); (13086 13486841 A T); (13087
13486871 G A); (13088 13486893 A G); (13089 13487028 A G); (13090 13487237 G A); (13091 13487260
A G); (13092 13487613 CT C); (13093 13487654 CAT C); (13094 13487910 T C); (13095 13488059 C
T); (13096 13488537 G A); (13097 13488699 C T); (13098 13489001 G A); (13099 13489700 T G); (13100
13489767 G GA); (13101 13490038 A G); (13102 13490441 G A); (13103 13490445 C
CAATAATAATAAT); (13104 13490698 C T); (13105 13492154 A G); (13106 13492516 G A); (13107
13493086 A G); (13108 13493240 C T); (13109 13493292 A G); (13110 13493400 C T); (13111 13493887
C T); (13112 13494026 T C); (13113 13494157 C A); (13114 13494417 G A); (13115 13494444 T C); (13116
13495147 C T); (13117 13495241 T C); (13118 13495417 C CAA); (13119 13495421 C G); (13120
13495921 C T); (13121 13496123 G A); (13122 13496746 G A); (13123 13496957 T C); (13124 13496979
T A); (13125 13497121 G C); (13126 13497537 G A); (13127 13497666 G A); (13128 13497706 G
A); (13129 13498375 A G); (13130 13498662 C A); (13131 13498772 A G); (13132 13498821 C T); (13133
13498975 A C); (13134 13499389 A G); (13135 13500811 G A); (13136 13501146 G A); (13137 13502281
C T); (13138 13502983 T G); (13139 13503168 T C); (13140 13503284 C A); (13141 13503598 A
AG); (13142 13503708 A G); (13143 13504051 AC A); (13144 13504123 T A); (13145 13504169 A
G); (13146 13504953 G T); (13147 13504999 T C); (13148 13505048 A G); (13149 13505083 C T); (13150
13505146 G T); (13151 13505337 A T); (13152 13506644 T C); (13153 13506661 C G); (13154 13507276
TTC); (13155 13507424 C T); (13156 13507962 A G); (13157 13508198 G A); (13158 13508213 G
A); (13159 13508402 C T); (13160 13508903 G A); (13161 13508920 T A); (13162 13509052 A G); (13163
13509200 G A); (13164 13509741 A G); (13165 13509758 C T); (13166 13510549 G A); (13167 13510592
A G); (13168 13510741 A T); (13169 13511035 T G); (13170 13511231 T C); (13171 13511534 T C); (13172
13511727 C A); (13173 13512105 A T); (13174 13512196 T C); (13175 13512325 G A); (13176 13512390
G A); (13177 13512530 C T); (13178 13512933 G T); (13179 13512984 GA G); (13180 13513443 A
AT); (13181 13513560 A G); (13182 13513743 GA G); (13183 13513969 TA T); (13184 13514394 A
G); (13185 13514595 C T); (13186 13514630 T A); (13187 13515036 G A); (13188 13515048 G T); (13189
13515100 C T); (13190 13515335 C T); (13191 13515469 G A); (13192 13515513 A G); (13193 13515541
C T); (13194 13515567 C T); (13195 13515579 C T); (13196 13516160 G A); (13197 13517096 C T); (13198
13518132 T TA); (13199 13518584 G A); (13200 13519023 C CAAACCCT); (13201 13519298 T C); (13202
13519438 G A); (13203 13519760 G C); (13204 13519786 C T); (13205 13519884 G A); (13206 13519950
C T); (13207 13520051 A G); (13208 13520123 G A); (13209 13520477 C T); (13210 13520606 T
G); (13211 13520688 C T); (13212 13520714 G GT); (13213 13520842 C T); (13214 13520853 C T); (13215
13521038 C T); (13216 13521263 A T); (13217 13521328 A G); (13218 13521371 T C); (13219 13521549
A G); (13220 13521560 A C); (13221 13521644 G C); (13222 13521705 A G); (13223 13521713 C
T); (13224 13522040 A C); (13225 13522232 A G); (13226 13522865 A G); (13227 13523359 A C); (13228
13523745 C G); (13229 13523833 G A); (13230 13523923 A G); (13231 13524129 C T); (13232 13524574
C T); (13233 13524645 G A); (13234 13525105 T C); (13235 13525188 C T); (13236 13525351 T C); (13237
13525688 T C); (13238 13526020 T C); (13239 13526054 T C); (13240 13526518 G T); (13241 13526794 C
T); (13242 13527000 A ATC); (13243 13527110 A G); (13244 13527119 G A); (13245 13527279 C
T); (13246 13527504 T A); (13247 13527620 T G); (13248 13527959 T G); (13249 13529154 A C); (13250
13529156 G GTGTAATGTTAATATTTTTTT); (13251 13529897 C T); (13252 13530454 T C); (13253
13530704 G A); (13254 13531327 A G); (13255 13531639 C T); (13256 13531768 T TA); (13257 13532101
T C); (13258 13532237 G A); (13259 13532247 G A); (13260 13532259 A T); (13261 13532528 G
A); (13262 13533567 G T); (13263 13533783 G A); (13264 13534087 T G); (13265 13534164 A C); (13266
13535107 T C); (13267 13535149 A G); (13268 13535246 C T); (13269 13535580 G A); (13270 13535886
C T); (13271 13536501 G T); (13272 13536519 C T); (13273 13536612 T C); (13274 13536947 G A); (13275
13538086 G C); (13276 13538116 T C); (13277 13538674 C T); (13278 13538848 C T); (13279 13538949 A
G); (13280 13538986 C T); (13281 13539526 A G); (13282 13539674 C T); (13283 13539860 G A); (13284
13540170 T C); (13285 13540989 A G); (13286 13541020 G A); (13287 13541044 G A); (13288 13541887
A G); (13289 13542402 T G); (13290 13542441 T C); (13291 13542559 A C); (13292 13543401 G
A); (13293 13543972 C T); (13294 13544046 C G); (13295 13544239 G A); (13296 13546019 C T); (13297
13546052 C T); (13298 13546697 A G); (13299 13546957 G T); (13300 13547574 T C); (13301 13548917
T C); (13302 13549067 T C); (13303 13549179 C T); (13304 13549461 T TC); (13305 13549761 C
T); (13306 13549905 T TAGA); (13307 13549944 C A); (13308 13549951 A T); (13309 13550448 A
G); (13310 13550722 A G); (13311 13550774 C A); (13312 13550859 G C); (13313 13551375 T C); (13314
13551547 T C); (13315 13551616 A C); (13316 13551668 C T); (13317 13552734 C T); (13318 13552814 T
C); (13319 13553117 A T); (13320 13554318 T C); (13321 13555043 A C); (13322 13555084 A G); (13323
13555142 T C); (13324 13555168 C T); (13325 13555947 A G); (13326 13556110 A C); (13327 13556545
C A); (13328 13556568 C A); (13329 13556640 A G); (13330 13557045 G T); (13331 13557700 T
C); (13332 13557728 G T); (13333 13557798 C T); (13334 13557815 T C); (13335 13557919 A G); (13336
13558374 G A); (13337 13558665 G A); (13338 13558926 T C); (13339 13559385 G T); (13340 13559545
G C); (13341 13560687 A G); (13342 13562100 C T); (13343 13562213 T C); (13344 13562368 T C); (13345
13563021 C T); (13346 13563209 G T); (13347 13563741 T C); (13348 13564437 T C); (13349 13564487
G A); (13350 13564590 T C); (13351 13564617 T C); (13352 13564911 T C); (13353 13566338 T
TGCAA); (13354 13566356 A G); (13355 13566491 A G); (13356 13566707 A G); (13357 13566876 T
C); (13358 13567273 C T); (13359 13567445 A AC); (13360 13567552 C T); (13361 13568294 TC
T); (13362 13568386 C T); (13363 13568423 C T); (13364 13568435 T C); (13365 13568482 G C); (13366
13568488 T C); (13367 13568792 A C); (13368 13569055 T C); (13369 13569083 G A); (13370 13569084
G A); (13371 13569190 G A); (13372 13569195 G C); (13373 13569198 T G); (13374 13569253 T
G); (13375 13569261 T C); (13376 13569302 C T); (13377 13570219 A G); (13378 13570624 C T); (13379
13570727 G C); (13380 13570792 T C); (13381 13570936 C T); (13382 13571089 T TA); (13383 13571110
T G); (13384 13571125 G A); (13385 13571231 T C); (13386 13571275 G A); (13387 13571301 C
G); (13388 13571597 C T); (13389 13571899 G A); (13390 13572582 T C); (13391 13572835 G A); (13392
13572981 A G); (13393 13573436 A T); (13394 13574385 A C); (13395 13574421 C T); (13396 13575791
A G); (13397 13575822 G T); (13398 13576130 C T); (13399 13576790 A G); (13400 13576794 C
T); (13401 13577854 C A); (13402 13578030 G A); (13403 13579953 T C); (13404 13580812 A G); (13405
13580936 C A); (13406 13582046 A G); (13407 13582270 T C); (13408 13582438 A G); (13409 13582574
A G); (13410 13582663 C A); (13411 13582699 G T); (13412 13582909 C T); (13413 13583319 T
C); (13414 13583328 G A); (13415 13583443 G A); (13416 13583720 C A); (13417 13584191 G A); (13418
13584227 C CA); (13419 13584275 G C); (13420 13584354 T C); (13421 13584393 A G); (13422
13584571 A C); (13423 13584721 T C); (13424 13584771 T C); (13425 13585262 C T); (13426 13585352 T
C); (13427 13585356 A G); (13428 13585549 T G); (13429 13585762 C A); (13430 13586191 A G); (13431
13586599 G A); (13432 13587089 C T); (13433 13587504 A G); (13434 13587795 C T); (13435 13587857
T C); (13436 13588270 C G); (13437 13599512 C G); (13438 13599700 A G); (13439 13600196 C
T); (13440 13600348 A G); (13441 13600423 A G); (13442 13601231 T C); (13443 13601279 T G); (13444
13601729 T C); (13445 13602137 T C); (13446 13602316 T C); (13447 13602321 C T); (13448 13602934 T
C); (13449 13603446 A G); (13450 13603510 T C); (13451 13603708 T C); (13452 13603806 T C); (13453
13604182 A G); (13454 13605607 C A); (13455 13605658 C T); (13456 13605770 C A); (13457 13606002
C T); (13458 13606389 A G); (13459 13606663 A T); (13460 13606925 G A); (13461 13607576 T
A); (13462 13607698 A G); (13463 13607730 T A); (13464 13607946 G T); (13465 13608057 G C); (13466
13608090 G A); (13467 13608127 CT C); (13468 13608161 A G); (13469 13608513 G A); (13470
13608857 C T); (13471 13608922 T C); (13472 13613929 A C); (13473 13618955 A AG); (13474 13619229
T C); (13475 13619429 T G); (13476 13619438 T C); (13477 13619461 A G); (13478 13619754 G
A); (13479 13619813 T G); (13480 13619850 T C); (13481 13619936 A G); (13482 13620003 G A); (13483
13620039 A G); (13484 13620061 T C); (13485 13620179 CT C); (13486 13621198 T C); (13487 13621271
T C); (13488 13621594 T C); (13489 13621818 C T); (13490 13621963 AT A); (13491 13622044 G
A); (13492 13622672 G A); (13493 13623537 T C); (13494 13623992 C T); (13495 13624011 G T); (13496
13624075 T C); (13497 13624531 T C); (13498 13625056 T C); (13499 13625165 G A); (13500 13625507
G A); (13501 13625841 A G); (13502 13625853 A C); (13503 13626175 T C); (13504 13627167 A
C); (13505 13628197 T C); (13506 13628206 C T); (13507 13628460 T A); (13508 13628585 A T); (13509
13628812 G A); (13510 13629867 G A); (13511 13630047 C T); (13512 13630851 G T); (13513 13631439
C T); (13514 13631450 C A); (13515 13631494 T C); (13516 13631517 C G); (13517 13632262 C T); (13518
13632307 G A); (13519 13632938 T C); (13520 13633307 A C); (13521 13633321 G A); (13522 13633525
C G); (13523 13633531 C A); (13524 13634739 T C); (13525 13634791 T TC); (13526 13635294 T
C); (13527 13635440 T C); (13528 13635624 G A); (13529 13635713 C T); (13530 13635861 T G); (13531
13635947 T C); (13532 13636078 T A); (13533 13636169 G A); (13534 13636778 A T); (13535 13636913
A T); (13536 13637194 C T); (13537 13637317 T TTA); (13538 13637328 G A); (13539 13637644 T
TA); (13540 13637867 AC A); (13541 13638125 A AT); (13542 13638733 C T); (13543 13638838 A
AGAAAAAAAGT); (13544 13639591 G A); (13545 13639768 A C); (13546 13639832 C T); (13547
13639879 C T); (13548 13640322 A T); (13549 13640670 T G); (13550 13640877 G A); (13551 13642255
G A); (13552 13642293 T C); (13553 13643326 C T); (13554 13644253 TA T); (13555 13644380 A
C); (13556 13644416 G C); (13557 13644688 T A); (13558 13644854 C T); (13559 13644937 T TA); (13560
13645616 C T); (13561 13645702 T G); (13562 13645767 C A); (13563 13646306 A ATGT); (13564
13646922 A G); (13565 13647366 T C); (13566 13647386 C A); (13567 13647560 C T); (13568 13647582
C T); (13569 13650655 T TTC); (13570 13650693 G A); (13571 13651049 T C); (13572 13651086 G
A); (13573 13652291 G A); (13574 13652319 T G); (13575 13652568 C T); (13576 13652945 G A); (13577
13653301 C A); (13578 13653504 A C); (13579 13654063 T C); (13580 13654087 T C); (13581 13654562 T
G); (13582 13654579 G A); (13583 13655287 T C); (13584 13655323 G A); (13585 13655706 AT
A); (13586 13655917 A G); (13587 13656412 C CTG); (13588 13656607 A G); (13589 13656727 T
C); (13590 13657975 G A); (13591 13658116 C T); (13592 13658271 G C); (13593 13658345 T C); (13594
13658363 C A); (13595 13658496 T C); (13596 13658636 A T); (13597 13658755 A G); (13598 13658793
C T); (13599 13658941 C T); (13600 13658981 C T); (13601 13658992 G A); (13602 13660198 T C); (13603
13660199 A G); (13604 13660285 G A); (13605 13660452 C G); (13606 13660603 C T); (13607 13661166
T C); (13608 13661293 G A); (13609 13661465 T C); (13610 13661562 T C); (13611 13661849 G
A); (13612 13662123 G A); (13613 13662254 C T); (13614 13662507 A G); (13615 13662626 A G); (13616
13662768 T C); (13617 13662828 G A); (13618 13664706 A G); (13619 13664959 C T); (13620 13665140
T C); (13621 13665264 A G); (13622 13665431 C T); (13623 13665446 C A); (13624 13665575 G
A); (13625 13666109 T A); (13626 13667337 T G); (13627 13667347 A G); (13628 13667385 A G); (13629
13667571 A AT); (13630 13667840 TA T); (13631 13667963 G A); (13632 13668694 A G); (13633
13673059 T C); (13634 13673645 G A); (13635 13674319 AT A); (13636 13675201 G A); (13637
13675237 A G); (13638 13676244 G A); (13639 13676791 T C); (13640 13677135 C T); (13641 13677551
G A); (13642 13677561 C T); (13643 13677594 C T); (13644 13677900 T C); (13645 13677969 G
A); (13646 13678491 G A); (13647 13678538 C T); (13648 13679199 A G); (13649 13679515 T C); (13650
13679582 G A); (13651 13679612 A G); (13652 13679763 G T); (13653 13679870 G A); (13654 13679872
T C); (13655 13679932 G A); (13656 13680045 A G); (13657 13680057 G A); (13658 13680092 C
T); (13659 13680149 C G); (13660 13680305 T A); (13661 13680393 A G); (13662 13680664 T TA); (13663
13680710 G T); (13664 13680769 A G); (13665 13680797 G A); (13666 13680810 T C); (13667 13680924
A AT); (13668 13681090 T C); (13669 13681529 C T); (13670 13681544 A G); (13671 13682622 T
C); (13672 13682639 A G); (13673 13682708 C T); (13674 13682839 C T); (13675 13683318 G T); (13676
13683406 T C); (13677 13684366 T C); (13678 13684437 G A); (13679 13684552 T C); (13680 13684568 C
T); (13681 13684699 T C); (13682 13684760 C T); (13683 13684794 T C); (13684 13684945 G A); (13685
13685202 T C); (13686 13685237 A G); (13687 13685274 G C); (13688 13685474 G A); (13689 13685540
A G); (13690 13685559 C T); (13691 13685631 A G); (13692 13685992 G A); (13693 13686070 T
G); (13694 13686081 T C); (13695 13686103 T C); (13696 13686187 A G); (13697 13686235 C A); (13698
13686279 T C); (13699 13686617 T C); (13700 13686655 G A); (13701 13686676 A T); (13702 13686860 T
A); (13703 13686911 T A); (13704 13687151 T C); (13705 13687610 TTTTTGAGGCAG T); (13706
13688492 A AC); (13707 13688956 T C); (13708 13689192 C A); (13709 13689270 T C); (13710 13689693
C T); (13711 13689797 A G); (13712 13690044 G A); (13713 13690696 A C); (13714 13690713 G
C); (13715 13690764 T C); (13716 13690877 G A); (13717 13692276 A T); (13718 13693163 G A); (13719
13694054 C T); (13720 13694103 A G); (13721 13694496 T A); (13722 13694584 C G); (13723 13694603
G A); (13724 13694679 T C); (13725 13695031 G A); (13726 13695535 C G); (13727 13696255 T
C); (13728 13696665 A C); (13729 13696984 T G); (13730 13696988 C T); (13731 13697126 C T); (13732
13697195 G A); (13733 13697201 T C); (13734 13697270 A C); (13735 13697431 T C); (13736 13697545
C T); (13737 13698184 A G); (13738 13698305 C G); (13739 13698361 G T); (13740 13698792 A
G); (13741 13698872 A G); (13742 13699157 G A); (13743 13699332 T G); (13744 13699762 G A); (13745
13700468 A C); (13746 13700531 G A); (13747 13700645 G A); (13748 13700828 A C); (13749 13700884
G A); (13750 13701149 C A); (13751 13701189 C T); (13752 13702280 A AAAT); (13753 13702307 A
C); (13754 13702883 A G); (13755 13703452 G T); (13756 13703481 A G); (13757 13704046 A G); (13758
13705743 A T); (13759 13706184 G A); (13760 13706186 T C); (13761 13706448 G A); (13762 13706663
A G); (13763 13706986 C A); (13764 13707024 C T); (13765 13707057 A T); (13766 13707097 A
G); (13767 13707398 T A); (13768 13707884 C G); (13769 13708954 C T); (13770 13709601 A G); (13771
13712105 T A); (13772 13712624 T G); (13773 13712629 C CA); (13774 13712643 C A); (13775 13712805
A G); (13776 13712806 G T); (13777 13715971 C A); (13778 13716673 T C); (13779 13716704 T
G); (13780 13729274 G T); (13781 13729872 G A); (13782 13731712 A T); (13783 13731863 C T); (13784
13732834 T C); (13785 13733256 G A); (13786 13733455 T A); (13787 13734217 C T); (13788 13734280 T
C); (13789 13734571 T C); (13790 13734617 G A); (13791 13734669 G A); (13792 13736052 T C); (13793
13737361 A G); (13794 13737733 A G); (13795 13739616 T C); (13796 13740436 TC T); (13797 13740875
C T); (13798 13741131 C T); (13799 13741415 T C); (13800 13741641 G A); (13801 13741804 G
A); (13802 13741822 T C); (13803 13741891 T C); (13804 13741923 G A); (13805 13741943 A T); (13806
13742298 T C); (13807 13742744 G A); (13808 13743619 G A); (13809 13743703 T C); (13810 13743761
T C); (13811 13743827 G A); (13812 13744170 G T); (13813 13744200 A G); (13814 13744232 G
A); (13815 13744355 A G); (13816 13744410 A G); (13817 13744744 C T); (13818 13744872 A G); (13819
13745111 A G); (13820 13745197 T C); (13821 13745289 G A); (13822 13745988 T G); (13823 13746153
T C); (13824 13746272 A G); (13825 13746408 A G); (13826 13746481 G T); (13827 13746688 T
C); (13828 13746821 T C); (13829 13747249 T C); (13830 13747549 G A); (13831 13747560 G T); (13832
13748092 A G); (13833 13748278 T C); (13834 13748290 T C); (13835 13748683 C G); (13836 13748738
T C); (13837 13748761 A T); (13838 13748912 T C); (13839 13749173 G A); (13840 13749523 G
A); (13841 13749524 T C); (13842 13749608 C T); (13843 13749857 G A); (13844 13749917 T C); (13845
13750498 A C); (13846 13750618 G A); (13847 13751529 T C); (13848 13752062 A G); (13849 13752177
G A); (13850 13752316 T C); (13851 13753331 A G); (13852 13753337 A G); (13853 13754568 C
T); (13854 13754853 A G); (13855 13754854 T C); (13856 13754904 A G); (13857 13755280 A G); (13858
13755330 G A); (13859 13755778 C T); (13860 13755869 A G); (13861 13755977 G A); (13862 13756741
T C); (13863 13756783 T C); (13864 13757096 T C); (13865 13757141 T TCCGCTTCTCCA); (13866
13757177 T C); (13867 13757209 A G); (13868 13757219 C T); (13869 13757279 G GGCCC); (13870
13760666 T G); (13871 13761384 A T); (13872 13762000 A AAATAGGGGG); (13873 13762071 G
A); (13874 13762577 CA C); (13875 13762901 A AATGAATT); (13876 13764153 G A); (13877 13765131 A
T); (13878 13766950 A G); (13879 13766991 T C); (13880 13768550 G C); (13881 13769792 A C); (13882
13771161 CT C); (13883 13771329 C A); (13884 13772281 A G); (13885 13773585 T C); (13886 13775942
G A); (13887 13776045 C T); (13888 13776277 C A); (13889 13777460 A G); (13890 13777555 T
C); (13891 13777977 A G); (13892 13778494 A G); (13893 13778620 C T); (13894 13778879 T C); (13895
13779234 A G); (13896 13780025 G A); (13897 13784668 G T); (13898 13789856 G A); (13899 13789880
A C); (13900 13792392 A G); (13901 13793553 C G); (13902 13793555 T C); (13903 13793706 C
T); (13904 13794108 C T); (13905 13794120 C T); (13906 13794313 C T); (13907 13794746 C T); (13908
13795228 A G); (13909 13795259 G A); (13910 13796074 G A); (13911 13796084 A G); (13912 13796200
C T); (13913 13796625 C A); (13914 13796769 T C); (13915 13796991 A G); (13916 13797045 C T); (13917
13797245 T A); (13918 13797304 C T); (13919 13797347 C T); (13920 13797398 T C); (13921 13797668 C
A); (13922 13797911 A G); (13923 13798418 G T); (13924 13798541 T G); (13925 13799319 T C); (13926
13799864 C G); (13927 13800181 T G); (13928 13800238 A T); (13929 13801519 A G); (13930 13802181
A G); (13931 13803421 T C); (13932 13806156 C T); (13933 13807277 C T); (13934 13807895 A
G); (13935 13808647 T A); (13936 13810279 A G); (13937 13810777 T G); (13938 13810918 G A); (13939
13811342 T C); (13940 13812936 T C); (13941 13814457 G A); (13942 13815538 G A); (13943 13817224
T C); (13944 13817599 T C); (13945 13818138 C T); (13946 13818328 C T); (13947 13818928 T C); (13948
13818931 T C); (13949 13819608 TA T); (13950 13820528 G A); (13951 13820530 T C); (13952 13821307
C T); (13953 13822123 G A); (13954 13822380 A C); (13955 13824830 G A); (13956 13825200 T
C); (13957 13825680 C T); (13958 13826896 C T); (13959 13827248 C T); (13960 13827602 C T); (13961
13828028 A C); (13962 13829251 C T); (13963 13830533 G C); (13964 13831297 A G); (13965 13831633
C T); (13966 13831684 A G); (13967 13831960 G C); (13968 13832067 G A); (13969 13832204 C
T); (13970 13832219 A G); (13971 13832737 A G); (13972 13832814 G A); (13973 13832824 C T); (13974
13832880 T C); (13975 13832975 G A); (13976 13833149 G T); (13977 13833950 T C); (13978 13834557
C T); (13979 13835004 A G); (13980 13835202 CT C); (13981 13835363 T C); (13982 13836169 CA
C); (13983 13836617 G C); (13984 13839056 T TAC); (13985 13839560 T G); (13986 13839903 A
G); (13987 13840316 C CATAT); (13988 13842118 A G); (13989 13842314 G A); (13990 13843394 C
T); (13991 13844198 A G); (13992 13844557 T G); (13993 13844959 TG T); (13994 13845475 A G); (13995
13846405 G T); (13996 13846882 T TA); (13997 13847615 A T); (13998 13849805 AT A); (13999
13849961 C G); (14000 13850319 A G); (14001 13850349 C T); (14002 13850987 G A); (14003 13854194
A AAAT); (14004 13856558 A G); (14005 13856766 C T); (14006 13856793 A G); (14007 13857822 G
A); (14008 13859645 G A); (14009 13860195 T C); (14010 13860259 G A); (14011 13860937 A G); (14012
13861940 G T); (14013 13861951 G C); (14014 13862427 G A); (14015 13862429 G A); (14016 13863377
G T); (14017 13863546 C A); (14018 13863911 T C); (14019 13864416 C T); (14020 13865020 T C); (14021
13866255 C T); (14022 13866586 T C); (14023 13866618 G C); (14024 13866736 A C); (14025 13866824
C T); (14026 13869836 C A); (14027 13869908 AT A); (14028 13870180 C T); (14029 13870211 G
A); (14030 13870233 G C); (14031 13871984 T C); (14032 13872589 A G); (14033 13873099 G A); (14034
13873163 T G); (14035 13873218 G C); (14036 13873290 C T); (14037 13873315 T A); (14038 13873340
A C); (14039 13873492 A T); (14040 13873823 C T); (14041 13875446 AG A); (14042 13876021 T
TTTGTTGTTGTTGTTG); (14043 13877105 T C); (14044 13877212 G T); (14045 13878030 C T); (14046
13878361 A G); (14047 13879381 C T); (14048 13879595 G A); (14049 13879649 A C); (14050 13879702
C T); (14051 13880183 C T); (14052 13882164 C A); (14053 13882470 G A); (14054 13882690 A T); (14055
13882811 A G); (14056 13883088 T C); (14057 13883159 G T); (14058 13883274 G A); (14059 13887179
C T); (14060 13889275 G A); (14061 13889420 C T); (14062 13890414 C A); (14063 13890446 T
G); (14064 13891884 C T); (14065 13892451 T TAAATAA); (14066 13892554 T A); (14067 13892572 T
C); (14068 13892611 A G); (14069 13892641 G T); (14070 13894021 G T); (14071 13897547 T A); (14072
13897817 T A); (14073 13897818 T A); (14074 13898548 C T); (14075 13899419 C T); (14076 13899488
AATACCACAAATTTGAT A); (14077 13900943 T C); (14078 13901439 G A); (14079 13901474 G C); (14080
13901879 A G); (14081 13901977 A G); (14082 13902159 G A); (14083 13902378 G A); (14084 13903073
T C); (14085 13903466 G A); (14086 13905616 G GT); (14087 13906025 A G); (14088 13906176 C
T); (14089 13907632 A G); (14090 13907708 A G); (14091 13908339 T G); (14092 13910058 C A); (14093
13910963 A G); (14094 13911434 G A); (14095 13911631 A T); (14096 13912298 T G); (14097 13912340
T C); (14098 13912448 C T); (14099 13912502 A G); (14100 13913063 G A); (14101 13913538 C
T); (14102 13914647 T G); (14103 13914917 G A); (14104 13916568 G A); (14105 13916797 G A); (14106
13916839 C T); (14107 13919347 T C); (14108 13919558 T G); (14109 13919649 G T); (14110 13920669 T
C); (14111 13921686 T C); (14112 13922461 T G); (14113 13924424 C T); (14114 13924684 C G); (14115
13924730 T G); (14116 13925597 A G); (14117 13925869 C A); (14118 13926041 G A); (14119 13929822
A T); (14120 13930100 A T); (14121 13930112 G A); (14122 13930571 A G); (14123 13931467 T
C); (14124 13932435 A G); (14125 13932736 G A); (14126 13933573 G A); (14127 13934428 C T); (14128
13934928 G A); (14129 13936401 G T); (14130 13936596 A G); (14131 13936736 T C); (14132 13937209
T G); (14133 13937460 GCAAAATC G); (14134 13939252 G T); (14135 13939536 G A); (14136 13942320
A G); (14137 13943260 G A); (14138 13944393 T C); (14139 13946921 A G); (14140 13947290 A
G); (14141 13947818 G A); (14142 13948434 G C); (14143 13948701 AAG A); (14144 13949439 G
A); (14145 13952881 C G); (14146 13952898 A G); (14147 13956285 CA C); (14148 13956776 C T); (14149
13957258 A G); (14150 13958611 A T); (14151 13959207 T C); (14152 13959606 T A); (14153 13960391
A T); (14154 13960472 C A); (14155 13963433 G A); (14156 13963485 T C); (14157 13963896 A
T); (14158 13964058 A G); (14159 13964147 T G); (14160 13964527 A G); (14161 13965510 G A); (14162
13967863 T G); (14163 13967996 A C); (14164 13968611 T C); (14165 13969564 A T); (14166 13971016
C T); (14167 13971874 A G); (14168 13973617 T G); (14169 13974321 C A); (14170 13974577 C
A); (14171 13974630 G A); (14172 13974720 AAAATG A); (14173 13976384 T A); (14174 13976819 A
C); (14175 13976953 T C); (14176 13977376 T A); (14177 13977490 A AAC); (14178 13978025 ATG
A); (14179 13980845 CGTAG C); (14180 13981747 G A); (14181 13982424 A G); (14182 13982510 A
AC); (14183 13982945 G T); (14184 13983338 T C); (14185 13983354 A G); (14186 13984370 C T); (14187
13984680 C G); (14188 13984719 T C); (14189 13984825 C T); (14190 13985431 CA C); (14191 13985678
T G); (14192 13986121 C T); (14193 13986645 C T); (14194 13987540 T A); (14195 13987966 C T); (14196
13987984 A T); (14197 13988002 G A); (14198 13988158 C T); (14199 13988185 T C); (14200 13988287 T
C); (14201 13988311 GC G); (14202 13990047 T C); (14203 13990100 C T); (14204 13990103 T C); (14205
13990144 T C); (14206 13990341 A G); (14207 13990379 T A); (14208 13991193 T G); (14209 13997724
G T); (14210 13997737 A G); (14211 13997773 A T); (14212 13997790 C T); (14213 14000758 T C); (14214
14000832 A T); (14215 14000930 T C); (14216 14000947 G A); (14217 14000950 C T); (14218 14000976
G C); (14219 14001453 C T); (14220 14003199 G A); (14221 14003703 A G); (14222 14003896 T
C); (14223 14004221 G A); (14224 14004497 C T); (14225 14005032 G T); (14226 14005054 T C); (14227
14005269 T G); (14228 14005312 A G); (14229 14005716 T C); (14230 14006293 A T); (14231 14006296
T C); (14232 14007319 A G); (14233 14008898 C T); (14234 14009295 A T); (14235 14010610 A T); (14236
14010768 T C); (14237 14010779 A T); (14238 14011009 T A); (14239 14011625 G A); (14240 14011749
T TA); (14241 14016733 C A); (14242 14018468 A G); (14243 14026068 AT A); (14244 14026297 T
C); (14245 14026960 A ATT); (14246 14027268 G C); (14247 14027303 C T); (14248 14027334 C
T); (14249 14027335 A G); (14250 14027351 C T); (14251 14027394 C T); (14252 14027411 G A); (14253
14027512 A G); (14254 14027921 A G); (14255 14028157 C T); (14256 14028393 A G); (14257 14029751
G A); (14258 14031254 T C); (14259 14031269 G GA); (14260 14031315 C T); (14261 14031462 G
T); (14262 14031476 C T); (14263 14032689 C A); (14264 14033886 A G); (14265 14034267 C G); (14266
14034293 T G); (14267 14034980 T C); (14268 14035026 T C); (14269 14035179 C T); (14270 14035529 A
G); (14271 14035822 C T); (14272 14036005 C G); (14273 14036149 A G); (14274 14036562 G
GA); (14275 14036630 G A); (14276 14036727 G A); (14277 14036924 GA G); (14278 14037204 C
A); (14279 14037311 T G); (14280 14037395 A G); (14281 14039163 A T); (14282 14039170 C A); (14283
14040437 A G); (14284 14041607 G C); (14285 14043786 GA G); (14286 14044938 A G); (14287
14045701 A G); (14288 14046567 G A); (14289 14046779 ATTTG A); (14290 14047127 T G); (14291
14048102 T TAG); (14292 14048105 A G); (14293 14048150 AT A); (14294 14048614 C T); (14295
14048679 A G); (14296 14048742 T C); (14297 14048827 A C); (14298 14049068 C T); (14299 14049214
C T); (14300 14050440 T C); (14301 14053603 C T); (14302 14053670 T G); (14303 14056913 GT
G); (14304 14060847 C T); (14305 14061513 T C); (14306 14062232 T C); (14307 14062267 A G); (14308
14062353 A C); (14309 14062462 C T); (14310 14062508 G A); (14311 14062639 C T); (14312 14062644
A G); (14313 14062775 G T); (14314 14063280 T A); (14315 14063283 T A); (14316 14063286 T
A); (14317 14063644 C T); (14318 14064600 T C); (14319 14064975 C A); (14320 14065116
TAATTTTTTTGTCATTTCTCA T); (14321 14065285 AT A); (14322 14065705 G T); (14323 14065922 T
TA); (14324 14066764 G A); (14325 14067413 T TA); (14326 14067515 A G); (14327 14067929 C
T); (14328 14068020 G T); (14329 14068294 G C); (14330 14068411 T G); (14331 14070490 G A); (14332
14076609 A C); (14333 14078280 C T); (14334 14079228 AC A); (14335 14079636 T A); (14336 14079864
C G); (14337 14079891 C T); (14338 14080002 T C); (14339 14080032 T A); (14340 14080751 A
AT); (14341 14080989 G A); (14342 14084026 T C); (14343 14084681 T G); (14344 14084794 A G); (14345
14086196 C T); (14346 14088955 T C); (14347 14088965 C T); (14348 14089074 C T); (14349 14089462 G
A); (14350 14093013 C G); (14351 14093266 A G); (14352 14093767 T A); (14353 14097490 G A); (14354
14102191 T C); (14355 14102953 G A); (14356 14104482 C T); (14357 14106124 T C); (14358 14106181 C
T); (14359 14107584 C CT); (14360 14110014 G C); (14361 14110824 A G); (14362 14112590 A G); (14363
14112635 A G); (14364 14114920 G A); (14365 14115798 C T); (14366 14116036 C T); (14367 14116330
C T); (14368 14116465 ATTACTAGATAACTCATTTATTG A); (14369 14116582 G A); (14370 14116639 AT
A); (14371 14117107 T C); (14372 14117134 C T); (14373 14117597 C G); (14374 14118470 T C); (14375
14118544 A G); (14376 14119498 G A); (14377 14119822 C T); (14378 14120450 A G); (14379 14121751
AATAT A); (14380 14122106 A G); (14381 14122884 T TA); (14382 14124767 G A); (14383 14125268 C
T); (14384 14128596 T G); (14385 14129343 G A); (14386 14131988 G C); (14387 14132039 C A); (14388
14134286 C T); (14389 14134814 C T); (14390 14135088 C T); (14391 14135971 T A); (14392 14136020
AT A); (14393 14137691 G T); (14394 14137754 T A); (14395 14138478 T C); (14396 14138704 T
C); (14397 14138868 T C); (14398 14144176 T TA); (14399 14144565 A T); (14400 14146191 A C); (14401
14148052 A G); (14402 14149173 TAAAAAGCCC T); (14403 14150260 A G); (14404 14150267 G
A); (14405 14152332 T TA); (14406 14152730 G A); (14407 14152864 T C); (14408 14154380 T A); (14409
14154842 C T); (14410 14155025 A G); (14411 14155114 A G); (14412 14155569 T TG); (14413
14156046 A G); (14414 14160747 A G); (14415 14160748 T C); (14416 14163302 G T); (14417 14163721
A G); (14418 14164293 C T); (14419 14165045 G A); (14420 14165060 G A); (14421 14165063 C
T); (14422 14165088 T G); (14423 14165109 T C); (14424 14168002 A G); (14425 14168423 C T); (14426
14169166 G A); (14427 14170101 C A); (14428 14170246 G GA); (14429 14170526 C
CCTGAATCCCCGCGCG); (14430 14172239 C T); (14431 14174406 G A); (14432 14176729 A G); (14433
14177093 C T); (14434 14177306 G A); (14435 14178490 G A); (14436 14180853 A G); (14437 14181242
T C); (14438 14181772 T C); (14439 14183836 G A); (14440 14186888 A G); (14441 14187030 A
G); (14442 14187189 T C); (14443 14189271 T C); (14444 14190329 A G); (14445 14191465 T C); (14446
14191743 G A); (14447 14191801 GA G); (14448 14193026 C T); (14449 14194458 T C); (14450
14195289 G A); (14451 14195371 G A); (14452 14196075 C CT); (14453 14197084 G A); (14454
14198402 A G); (14455 14198556 A G); (14456 14199578 T TA); (14457 14202060 G GA); (14458
14203273 G A); (14459 14203274 A T); (14460 14204709 A G); (14461 14205232 T C); (14462 14205260
A C); (14463 14205279 G A); (14464 14205491 A G); (14465 14205757 T C); (14466 14210713 G
A); (14467 14210848 G T); (14468 14212809 T C); (14469 14214620 G GTT); (14470 14214838 C
T); (14471 14216127 GA G); (14472 14217164 T G); (14473 14218038 T C); (14474 14218056 C T); (14475
14219670 A G); (14476 14219676 A G); (14477 14221880 G A); (14478 14221983 G T); (14479 14223497
A G); (14480 14223627 C T); (14481 14225185 A G); (14482 14225497 C A); (14483 14225567 C
T); (14484 14225621 A T); (14485 14226402 T C); (14486 14227225 T C); (14487 14227681 A T); (14488
14228802 T C); (14489 14233088 G A); (14490 14236579 G A); (14491 14238318 GA G); (14492
14238485 T C); (14493 14239294 C T); (14494 14240043 A G); (14495 14240199 C T); (14496 14241097 T
A); (14497 14241263 C T); (14498 14242625 G A); (14499 14242705 AT A); (14500 14243238 G C); (14501
14243909 T C); (14502 14244469 C G); (14503 14246043 TA T); (14504 14247238 C T); (14505 14248356
T C); (14506 14248526 T C); (14507 14248683 T C); (14508 14255007 G A); (14509 14255037 T C); (14510
14256907 G A); (14511 14257197 C T); (14512 14257387 A C); (14513 14257917 C T); (14514 14258051
C CA); (14515 14263056 T TATA); (14516 14263592 T G); (14517 14264619 C CTTTTCTAG); (14518
14266171 C T); (14519 14266255 G T); (14520 14267176 G A); (14521 14267206 C T); (14522 14267657
A G); (14523 14268847 G A); (14524 14268894 G A); (14525 14269798 G A); (14526 14269800 G
A); (14527 14271246 G C); (14528 14271250 T C); (14529 14271275 A C); (14530 14272465 CA C); (14531
14273899 G A); (14532 14274973 A T); (14533 14275045 A G); (14534 14275593 A T); (14535 14275689
T A); (14536 14275882 G A); (14537 14277599 C T); (14538 14277994 C T); (14539 14278287 T C); (14540
14278334 G A); (14541 14278695 C T); (14542 14279139 G A); (14543 14279851 TTA T); (14544
14282071 G T); (14545 14282452 G T); (14546 14286023 G A); (14547 14286677 C T); (14548 14290071
A T); (14549 14293004 T C); (14550 14293006 A G); (14551 14293286 C T); (14552 14294003 A
G); (14553 14295462 G A); (14554 14302245 T C); (14555 14303492 G A); (14556 14304896 A G); (14557
14310045 C G); (14558 14310653 A T); (14559 14310758 G A); (14560 14312175 C T); (14561 14312659
C T); (14562 14314972 T G); (14563 14316214 G A); (14564 14316525 A T); (14565 14318576 C T); (14566
14319421 C A); (14567 14319435 C T); (14568 14321162 T A); (14569 14321187 G A); (14570 14322623
C A); (14571 14322742 G A); (14572 14326137 A T); (14573 14326279 G A); (14574 14326337 A
G); (14575 14326822 T C); (14576 14328122 A G); (14577 14328802 A G); (14578 14333640 A T); (14579
14333834 A G); (14580 14335890 G GA); (14581 14338160 C T); (14582 14338392 G T); (14583
14338631 A G); (14584 14339946 C T); (14585 14340463 T C); (14586 14340485 C T); (14587 14340668
TA T); (14588 14340799 A G); (14589 14340907 T A); (14590 14340924 A C); (14591 14341008 C
G); (14592 14341045 G A); (14593 14341084 T C); (14594 14341296 AGTGAATG A); (14595 14343153 C
T); (14596 14343562 G A); (14597 14343812 C T); (14598 14344266 C T); (14599 14344680 T C); (14600
14345047 G A); (14601 14345523 A G); (14602 14346004 C T); (14603 14348317 T C); (14604 14350166
A T); (14605 14350174 C T); (14606 14350573 C T); (14607 14350760 T C); (14608 14351830 A G); (14609
14352342 C T); (14610 14357851 G A); (14611 14358098 T C); (14612 14358335 C G); (14613 14358526
C A); (14614 14361392 GA G); (14615 14364423 A G); (14616 14365646 C A); (14617 14366303 G
C); (14618 14366311 A T); (14619 14367525 TG T); (14620 14368458 T C); (14621 14368571 C T); (14622
14368990 A G); (14623 14369556 T C); (14624 14369574 C T); (14625 14370401 C T); (14626 14370435
A G); (14627 14371520 T A); (14628 14372693 C T); (14629 14373031 A C); (14630 14374001 G
A); (14631 14374413 G A); (14632 14375980 A G); (14633 14376650 C A); (14634 14379104 C T); (14635
14379723 G A); (14636 14379941 A C); (14637 14381045 T C); (14638 14381046 C T); (14639 14381892
C T); (14640 14381990 A G); (14641 14382254 C T); (14642 14385851 G A); (14643 14385881
CATCTGTGCTTGAGA C); (14644 14385961 C G); (14645 14387038 G A); (14646 14387511 T G); (14647
14388180 C T); (14648 14388274 C A); (14649 14389346 A G); (14650 14390345 A G); (14651 14401941
G C); (14652 14402985 G A); (14653 14403577 G A); (14654 14405131 G A); (14655 14405496 G
A); (14656 14405532 A G); (14657 14406669 TG T); (14658 14408326 G C); (14659 14408765 G
A); (14660 14410125 TC T); (14661 14410492 G T); (14662 14419857 A AT); (14663 14421227 C
T); (14664 14421586 C T); (14665 14421589 G A); (14666 14421639 T G); (14667 14421695 T C); (14668
14422464 G A); (14669 14423120 T G); (14670 14423579 G C); (14671 14423852 C A); (14672 14425078
A T); (14673 14425709 C T); (14674 14426059 TCTA T); (14675 14426649 A C); (14676 14427278 A
G); (14677 14428124 A G); (14678 14428402 G A); (14679 14428504 C T); (14680 14429145 T C); (14681
14430456 T C); (14682 14431153 A C); (14683 14433306 T TA); (14684 14435191 A AT); (14685
14435453 A G); (14686 14436044 G A); (14687 14436345 T TA); (14688 14436472 T C); (14689 14437160
C G); (14690 14439005 T C); (14691 14440123 A G); (14692 14440909 T A); (14693 14441481 A
G); (14694 14441490 T A); (14695 14442017 G A); (14696 14442361 C G); (14697 14443333 A G); (14698
14444177 A C); (14699 14444386 AT A); (14700 14444653 T C); (14701 14445344 TA T); (14702
14445477 A G); (14703 14445518 GT G); (14704 14445895 A AT); (14705 14446342 G A); (14706
14446678 AT A); (14707 14448394 GT G); (14708 14448619 T A); (14709 14448699 A G); (14710
14449846 C T); (14711 14450218 T C); (14712 14450252 C T); (14713 14450618 G A); (14714 14452190 T
TGGGTTTAGGGTTTA); (14715 14456050 C T); (14716 14458716 C T); (14717 14458963 G A); (14718
14459609 C T); (14719 14459616 C T); (14720 14459814 T C); (14721 14459888 A G); (14722 14460805
G A); (14723 14461158 A T); (14724 14461237 T TA); (14725 14461439 A G); (14726 14461517 G
A); (14727 14461570 C CA); (14728 14461612 ATT A); (14729 14461681 A T); (14730 14463807 TA
T); (14731 14463991 G A); (14732 14464441 A T); (14733 14465543 G A); (14734 14465851 T G); (14735
14467764 T C); (14736 14467828 T TTATA); (14737 14468337 GT G); (14738 14468429 C A); (14739
14470342 T C); (14740 14471453 T C); (14741 14473032 G GT); (14742 14473816 G GAA); (14743
14475122 T A); (14744 14476833 T G); (14745 14477927 C T); (14746 14477997 T C); (14747 14481086 A
T); (14748 14481143 A G); (14749 14482774 G T); (14750 14487684 C T); (14751 14487904 A C); (14752
14488403 C G); (14753 14489833 A G); (14754 14489856 A G); (14755 14491343 A C); (14756 14491739
G A); (14757 14492260 G A); (14758 14492339 G A); (14759 14493178 T C); (14760 14494131 T
G); (14761 14495211 C T); (14762 14496238 G A); (14763 14496380 G A); (14764 14497581 C A); (14765
14501500 C T); (14766 14504035 G A); (14767 14506493 CA C); (14768 14508683 A G); (14769
14509250 C T); (14770 14510042 G C); (14771 14511917 G A); (14772 14513758 C T); (14773 14513956
G A); (14774 14515756 A G); (14775 14516887 G C); (14776 14516888 A G); (14777 14516889 A
G); (14778 14517018 CG C); (14779 14517070 C T); (14780 14517073 T C); (14781 14517785 T C); (14782
14518798 T C); (14783 14519783 T G); (14784 14519873 G T); (14785 14520700 C T); (14786 14521602
G A); (14787 14522446 T C); (14788 14526213 G A); (14789 14527038 G A); (14790 14527041 G
A); (14791 14527194 A G); (14792 14527736 C T); (14793 14527792 C T); (14794 14529057 A G); (14795
14533391 G C); (14796 14534894 T G); (14797 14539011 G A); (14798 14539025 A G); (14799 14539695
C A); (14800 14540986 A G); (14801 14541841 GA G); (14802 14543206 A C); (14803 14545588 AT
A); (14804 14547205 T A); (14805 14548777 G A); (14806 14554313 T G); (14807 14554527 G A); (14808
14554584 T A); (14809 14555008 C CT); (14810 14555963 T TA); (14811 14556272 CA C); (14812
14556728 TT AA); (14813 14558126 A G); (14814 14558283 A G); (14815 14559818 T TA); (14816
14559891 G T); (14817 14560183 G A); (14818 14560322 G A); (14819 14560396 T A); (14820 14560474
TA T); (14821 14561373 TA T); (14822 14567603 A G); (14823 14568617 C T); (14824 14569213 GTATA
G); (14825 14569561 A C); (14826 14569589 A T); (14827 14569593 A T); (14828 14569662 C A); (14829
14570020 G A); (14830 14570893 C T); (14831 14571002 A G); (14832 14571436 C T); (14833 14571535
C T); (14834 14571747 G A); (14835 14572085 T C); (14836 14573188 T C); (14837 14575149 C G); (14838
14575383 A G); (14839 14575626 C T); (14840 14580759 G T); (14841 14580963 C T); (14842 14581911
G A); (14843 14582083 A C); (14844 14582137 G C); (14845 14585800 A C); (14846 14587428 A
G); (14847 14588479 C G); (14848 14590603 AG A); (14849 14591852 A G); (14850 14593109 G
A); (14851 14594159 C T); (14852 14594307 T G); (14853 14595297 G A); (14854 14595690 G C); (14855
14595727 C CA); (14856 14597848 A G); (14857 14603422 TA T); (14858 14607603 T G); (14859
14609092 C T); (14860 14609307 T C); (14861 14611823 C A); (14862 14614939 GTATTTT G); (14863
14617802 G C); (14864 14617906 A G); (14865 14619033 A G); (14866 14619075 C T); (14867 14620588
C T); (14868 14620667 T G); (14869 14620745 A C); (14870 14621897 A C); (14871 14622001 A
C); (14872 14622232 A G); (14873 14623556 T C); (14874 14623557 C T); (14875 14623663 TAC
T); (14876 14623677 C T); (14877 14623706 A G); (14878 14623709 A G); (14879 14625011 T A); (14880
14629340 AGCAAGATTGGTG A); (14881 14632507 T C); (14882 14632921 A G); (14883 14635693 G
A); (14884 14636751 C T); (14885 14637876 C T); (14886 14638614 G A); (14887 14638755 C G); (14888
14640088 C T); (14889 14640206 A T); (14890 14640511 T A); (14891 14642973 A G); (14892 14645439
T A); (14893 14646081 A G); (14894 14650604 A G); (14895 14650949 T C); (14896 14651322 A
T); (14897 14651398 C T); (14898 14652275 T A); (14899 14652470 G A); (14900 14654414 C T); (14901
14659148 T A); (14902 14660454 G A); (14903 14661835 A G); (14904 14661844 C A); (14905 14661861
G A); (14906 14662360 A G); (14907 14662692 A G); (14908 14663036 T C); (14909 14665766 A
C); (14910 14669494 T C); (14911 14669750 A G); (14912 14671812 TCA T); (14913 14672391 C
A); (14914 14674658 T C); (14915 14675825 C T); (14916 14675945 C G); (14917 14677975 C A); (14918
14678194 T C); (14919 14680165 G T); (14920 14680485 T TAC); (14921 14680507 T C); (14922
14680584 T C); (14923 14681276 C A); (14924 14681732 G A); (14925 14682023 T A); (14926 14685305
T A); (14927 14685404 ACTT A); (14928 14685787 T G); (14929 14688303 C T); (14930 14688515 C
A); (14931 14688550 TA T); (14932 14689261 T G); (14933 14689307 C T); (14934 14692082 T C); (14935
14693558 G A); (14936 14694183 C T); (14937 14694538 T C); (14938 14695199 TCA T); (14939
14697653 G A); (14940 14703702 G A); (14941 14704271 G A); (14942 14708366 A C); (14943 14708767
T C); (14944 14715112 T G); (14945 14723276 T C); (14946 14723280 C A); (14947 14723283 T C); (14948
14723287 G A); (14949 14723502 G A); (14950 14728048 A G); (14951 14728174 A G); (14952 14728896
T G); (14953 14730387 T A); (14954 14731016 T C); (14955 14731450 AG A); (14956 14731778 G
A); (14957 14731834 G A); (14958 14732221 T G); (14959 14732723 C CA); (14960 14734539 G
A); (14961 14736279 C T); (14962 14736492 A AT); (14963 14739400 A C); (14964 14740038 C T); (14965
14740197 C A); (14966 14740326 C T); (14967 14740498 A G); (14968 14745087 G A); (14969 14745105
T C); (14970 14745587 A G); (14971 14748522 A G); (14972 14750950 G T); (14973 14751298 G
T); (14974 14753789 G A); (14975 14753927 C T); (14976 14754200 T C); (14977 14755222 G A); (14978
14755362 T TGTGGCGCCAGGGGCGCCAGA); (14979 14755559 A C); (14980 14756512 T C); (14981
14758936 T C); (14982 14759005 C T); (14983 14760139 C T); (14984 14760541 T C); (14985 14761262 A
G); (14986 14761879 A G); (14987 14762847 T C); (14988 14763816 G A); (14989 14766949 T C); (14990
14768406 A G); (14991 14769463 T C); (14992 14771251 A C); (14993 14771519 C T); (14994 14771567
T C); (14995 14772193 CA C); (14996 14777316 C T); (14997 14778611 C T); (14998 14778812 T
C); (14999 14780460 C A); (15000 14780637 GTAATCGATTAT G); (15001 14781088 G GA); (15002
14781705 G C); (15003 14782200 T C); (15004 14783329 G A); (15005 14784514 T C); (15006 14785057
C T); (15007 14786735 A AC); (15008 14786908 A ACATCGATTTTT); (15009 14786977 AG A); (15010
14792871 C A); (15011 14792905 G A); (15012 14795831 G A); (15013 14799067 C T); (15014 14801955
C T); (15015 14802976 A G); (15016 14804032 T C); (15017 14804153 C T); (15018 14805466 G
GA); (15019 14805666 C A); (15020 14806137 AAT A); (15021 14807586 T C); (15022 14807662 G
C); (15023 14808097 A T); (15024 14808593 A AT); (15025 14811014 G A); (15026 14811211 A
AT); (15027 14811499 G A); (15028 14811500 G A); (15029 14814064 C T); (15030 14814703 G
A); (15031 14816466 G A); (15032 14816558 T C); (15033 14816613 G A); (15034 14816638 T C); (15035
14816665 G T); (15036 14817005 A G); (15037 14817104 G A); (15038 14819854 T C); (15039 14820038
T C); (15040 14820643 C A); (15041 14821230 A C); (15042 14822547 T C); (15043 14823575 C
CT); (15044 14823689 T C); (15045 14823959 A C); (15046 14826167 G GT); (15047 14826845 TA
T); (15048 14827233 G T); (15049 14827623 G A); (15050 14828761 A AT); (15051 14829132 T C); (15052
14829405 T C); (15053 14831024 G A); (15054 14831082 C T); (15055 14831531 T C); (15056 14831746
AT A); (15057 14834166 CA C); (15058 14834581 C T); (15059 14835808 T C); (15060 14839180 G
A); (15061 14839268 G A); (15062 14839739 A C); (15063 14840119 G A); (15064 14842842 C T); (15065
14842866 C T); (15066 14843452 A T); (15067 14844250 A T); (15068 14844540 G GT); (15069 14845222
AT A); (15070 14845400 C T); (15071 14848459 TCC T); (15072 14849422 G A); (15073 14852619 G
T); (15074 14855719 A T); (15075 14856103 AT A); (15076 14856990 ATTT A); (15077 14857573 A
G); (15078 14857647 A C); (15079 14858928 T G); (15080 14859525 G A); (15081 14859641 C T); (15082
14859713 T TGCCCCAAGCGTGGGTTCCCAC); (15083 14860580 G A); (15084 14862823 A T); (15085
14862956 T G); (15086 14863021 C T); (15087 14863368 C T); (15088 14864653 AT A); (15089 14864844
G C); (15090 14864994 G A); (15091 14865974 A T); (15092 14866167 G A); (15093 14866584 C
T); (15094 14866677 T TAA); (15095 14868367 C T); (15096 14869229 A C); (15097 14870003 A
C); (15098 14874279 T C); (15099 14874539 C T); (15100 14875906 A G); (15101 14875925 C A); (15102
14876284 G A); (15103 14876438 AG A); (15104 14876858 A G); (15105 14877078 A T); (15106
14877443 A C); (15107 14878301 A G); (15108 14878371 TA T); (15109 14878814 T G); (15110 14881915
A T); (15111 14886800 T C); (15112 14888046 C T); (15113 14890322 T C); (15114 14890592 A
AC); (15115 14890739 A AATATATAT); (15116 14890853 T C); (15117 14890870 TGTCTATCATTATTA
T); (15118 14890936 A G); (15119 14892045 C T); (15120 14892309 G C); (15121 14892499 T C); (15122
14893304 G C); (15123 14896156 T C); (15124 14896467 G A); (15125 14896619 G C); (15126 14897592
TTA T); (15127 14898398 C T); (15128 14898958 A AT); (15129 14899004 T C); (15130 14900359 G
A); (15131 14900371 G GT); (15132 14901855 G C); (15133 14904602 C T); (15134 14906341 C T); (15135
14906359 C T); (15136 14906815 C T); (15137 14907423 C T); (15138 14909018 G A); (15139 14909309
G A); (15140 14909668 C T); (15141 14909920 G A); (15142 14910402 A AT); (15143 14910732 G
A); (15144 14910851 A AC); (15145 14912699 G T); (15146 14913442 G A); (15147 14913741 C
G); (15148 14915004 C A); (15149 14915133 A T); (15150 14917001 G A); (15151 14917369 A G); (15152
14917447 A ACC); (15153 14918657 C T); (15154 14919578 C T); (15155 14919596 G A); (15156
14926626 G A); (15157 14928317 C CT); (15158 14929968 G A); (15159 14930530 T G); (15160
14931604 C CACAT); (15161 14934174 T C); (15162 14934878 C T); (15163 14935161 T A); (15164
14935210 A G); (15165 14939023 C T); (15166 14941145 A G); (15167 14942472 T C); (15168 14942578
G A); (15169 14944416 A AT); (15170 14946046 T C); (15171 14947465 G A); (15172 14947512 A
C); (15173 14947558 C T); (15174 14947748 C T); (15175 14947825 A G); (15176 14949503 G A); (15177
14949925 C T); (15178 14950505 C A); (15179 14950629 G GA); (15180 14951303 C T); (15181 14952687
A G); (15182 14952926 A G); (15183 14953158 GA G); (15184 14953172 T G); (15185 14953510 C
T); (15186 14953572 CA C); (15187 14953586 C T); (15188 14953794 CT C); (15189 14953797 C
G); (15190 14956423 C T); (15191 14957984 G A); (15192 14958626 G T); (15193 14962946 G A); (15194
14963196 A G); (15195 14963376 C T); (15196 14963428 T A); (15197 14963447 C T); (15198 14964198
A G); (15199 14965031 C A); (15200 14965086 C T); (15201 14965442 G A); (15202 14967873 G
T); (15203 14967940 C G); (15204 14968510 G A); (15205 14968538 G A); (15206 14970563 T C); (15207
14971684 G T); (15208 14971906 A C); (15209 14973738 G A); (15210 14977179 G A); (15211 14982473
A G); (15212 14982603 A C); (15213 14983418 A G); (15214 14984430 C T); (15215 14984488 G
A); (15216 14984774 C A); (15217 14984808 C T); (15218 14986461 C T); (15219 14986514 C A); (15220
14986702 T C); (15221 14986735 G A); (15222 14986873 C T); (15223 14987132 A C); (15224 14987271
T C); (15225 14987435 C T); (15226 14987466 G A); (15227 14989591 G A); (15228 14996330 T
C); (15229 14996332 A T); (15230 14996334 A T); (15231 15002243 G A); (15232 15006169 T A); (15233
15006704 T G); (15234 15009612 A T); (15235 15009756 A G); (15236 15010406 A AT); (15237
15012870 A G); (15238 15013932 G A); (15239 15014389 T C); (15240 15016144 T A); (15241 15016186
G A); (15242 15016246 G A); (15243 15016255 C T); (15244 15016377 C T); (15245 15016399 G
A); (15246 15016409 C T); (15247 15016432 C T); (15248 15016437 A G); (15249 15016443 A C); (15250
15016931 G A); (15251 15017632 G A); (15252 15017971 G T); (15253 15018290 T C); (15254 15018296
AG A); (15255 15018321 A G); (15256 15018333 A G); (15257 15019221 A G); (15258 15022502 G
A); (15259 15024149 G A); (15260 15024406 A C); (15261 15025706 A G); (15262 15025967 T C); (15263
15026306 G A); (15264 15026948 T C); (15265 15031415 GT G); (15266 15031451 C A); (15267
15035134 G T); (15268 15035139 A G); (15269 15035164 C A); (15270 15035202 C T); (15271 15036688
G A); (15272 15039427 A T); (15273 15043983 GT G); (15274 15044019 C A); (15275 15046737 C
T); (15276 15047185 A G); (15277 15047219 A G); (15278 15047281 G A); (15279 15047288 A T); (15280
15047483 C T); (15281 15047504 A G); (15282 15049160 C T); (15283 15055239 T C); (15284 15057264
G T); (15285 15057625 C T); (15286 15057635 T C); (15287 15059959 A C); (15288 15060038 T C); (15289
15060818 T A); (15290 15065101 A G); (15291 15065126 A T); (15292 15067303 A G); (15293 15071872
T C); (15294 15073756 G GA); (15295 15073892 A C); (15296 15077070 C A); (15297 15077432 G
T); (15298 15079615 C T); (15299 15080102 A C); (15300 15080186 A T); (15301 15080224 TAA
T); (15302 15080527 A G); (15303 15080809 T C); (15304 15081728 A G); (15305 15081963 G A); (15306
15081970 AC A); (15307 15082200 A C); (15308 15083027 A AC); (15309 15083088 A AT); (15310
15083524 A T); (15311 15085975 C T); (15312 15086507 T C); (15313 15087900 G A); (15314 15088359 T
G); (15315 15088596 A G); (15316 15089396 C T); (15317 15090581 C T); (15318 15094314 G T); (15319
15095555 G A); (15320 15095991 T G); (15321 15096178 T C); (15322 15096557 T C); (15323 15098083
T C); (15324 15099154 A C); (15325 15099226 T C); (15326 15099380 A G); (15327 15099456 G
A); (15328 15100137 A G); (15329 15101154 T C); (15330 15102897 C T); (15331 15103094 G A); (15332
15105548 C T); (15333 15105838 C T); (15334 15109162 G A); (15335 15111615 C T); (15336 15113739
A G); (15337 15113874 G A); (15338 15114569 G A); (15339 15117334 G A); (15340 15118533 G
T); (15341 15118761 G A); (15342 15119075 T A); (15343 15119409 T C); (15344 15121066 C G); (15345
15121226 A AC); (15346 15127321 A G); (15347 15127347 C G); (15348 15127478 C T); (15349
15127911 T C); (15350 15131105 G A); (15351 15131230 C T); (15352 15133016 C T); (15353 15135281 T
C); (15354 15138270 A AC); (15355 15141046 C G); (15356 15142030 G T); (15357 15144377 C T); (15358
15144476 C T); (15359 15144710 A G); (15360 15144794 G A); (15361 15147009 A ACT); (15362
15156775 G A); (15363 15157368 C T); (15364 15157427 A G); (15365 15160204 A G); (15366 15160478
C T); (15367 15161117 G A); (15368 15161121 A T); (15369 15161123 A ATT); (15370 15162020 A
T); (15371 15165012 G A); (15372 15165481 C T); (15373 15166157 T C); (15374 15169670 A AT); (15375
15169874 T TG); (15376 15170758 CA C); (15377 15170898 C T); (15378 15171016 A G); (15379
15171050 A T); (15380 15171688 T C); (15381 15175550 T G); (15382 15175610 C T); (15383 15175707 A
G); (15384 15176290 C T); (15385 15176855 G A); (15386 15177054 TAATG T); (15387 15177708 T
G); (15388 15177910 G A); (15389 15178951 G A); (15390 15178961 C T); (15391 15180686 C A); (15392
15181603 A G); (15393 15181692 G A); (15394 15182618 A G); (15395 15182948 A T); (15396 15183086
G A); (15397 15183237 C T); (15398 15183436 G A); (15399 15183649 C T); (15400 15183693 A
G); (15401 15183710 A T); (15402 15186894 T C); (15403 15188008 A C); (15404 15188383 G A); (15405
15190106 G A); (15406 15190875 A G); (15407 15190992 G A); (15408 15191984 C T); (15409 15192495
G T); (15410 15194883 C T); (15411 15194915 AT A); (15412 15196016 A G); (15413 15196090 G
A); (15414 15196194 C T); (15415 15196285 G C); (15416 15199228 A G); (15417 15201667 T C); (15418
15204800 T A); (15419 15206325 A G); (15420 15206890 G A); (15421 15207410 C A); (15422 15207448
G A); (15423 15207941 A G); (15424 15208424 G A); (15425 15209813 AGG A); (15426 15210093 CT
C); (15427 15210231 GA G); (15428 15210286 A AAAGTTTTAT); (15429 15210762 A G); (15430
15214037 C G); (15431 15214154 A G); (15432 15216446 T G); (15433 15218934 T C); (15434 15219501
ATG A); (15435 15219834 G A); (15436 15220102 C T); (15437 15220294 A G); (15438 15223943 A
G); (15439 15227885 C A); (15440 15228070 C T); (15441 15230195 TTA T); (15442 15230546 T
C); (15443 15231666 T G); (15444 15231998 G T); (15445 15234279 C T); (15446 15235082 C T); (15447
15235219 A T); (15448 15238322 G A); (15449 15238812 G A); (15450 15239909 C G); (15451 15241867
CAAAATATG C); (15452 15244483 T A); (15453 15244574 T TA); (15454 15248332 G T); (15455 15249139
T C); (15456 15256689 C T); (15457 15256758 T TC); (15458 15257309 C T); (15459 15257554 TC
T); (15460 15258860 A G); (15461 15259319 T C); (15462 15260033 AT A); (15463 15261536 T C); (15464
15261746 G C); (15465 15265033 A C); (15466 15266292 C T); (15467 15267112 T G); (15468 15267186
A G); (15469 15267414 C T); (15470 15267728 C T); (15471 15267733 C T); (15472 15267762 A
G); (15473 15267784 C T); (15474 15267842 T C); (15475 15267843 G A); (15476 15268132 T G); (15477
15270659 A G); (15478 15271909 G A); (15479 15271912 T G); (15480 15272013 C A); (15481 15272230
AG A); (15482 15272284 G A); (15483 15273073 T TTATA); (15484 15273428 CA C); (15485 15275611 T
C); (15486 15276054 G A); (15487 15276547 T C); (15488 15276855 T C); (15489 15276890 G A); (15490
15276945 C T); (15491 15277234 A G); (15492 15277642 AC A); (15493 15278341 A G); (15494
15279125 T C); (15495 15279758 G A); (15496 15279872 C T); (15497 15280088 C T); (15498 15281153 C
A); (15499 15281231 T C); (15500 15281525 C A); (15501 15282305 T C); (15502 15283280 C T); (15503
15283493 C T); (15504 15284806 T TA); (15505 15285082 T C); (15506 15285957 C T); (15507 15286080
T C); (15508 15286192 G A); (15509 15287274 C T); (15510 15288467 AC A); (15511 15289052 C
T); (15512 15291880 AT A); (15513 15292867 G A); (15514 15294152 T C); (15515 15295633 T C); (15516
15296328 C A); (15517 15299041 A C); (15518 15301305 C T); (15519 15303946 G A); (15520 15304074
G A); (15521 15304262 T C); (15522 15306230 C T); (15523 15306266 C T); (15524 15306931 A T); (15525
15306968 C T); (15526 15309772 C CGAATCGCGCG); (15527 15310576 T C); (15528 15311621 A
G); (15529 15312100 G A); (15530 15312725 G T); (15531 15312898 C T); (15532 15312901 A G); (15533
15314757 A G); (15534 15315455 T C); (15535 15316128 C A); (15536 15316616 T G); (15537 15317204
G A); (15538 15317609 G A); (15539 15317646 G T); (15540 15317648 T C); (15541 15318110 G
A); (15542 15318421 T C); (15543 15321828 A G); (15544 15322206 G A); (15545 15322272 G A); (15546
15322935 G T); (15547 15322970 A T); (15548 15324621 TA T); (15549 15326068 G A); (15550 15335054
A C); (15551 15335295 A G); (15552 15335328 G A); (15553 15336162 T C); (15554 15336219 T
C); (15555 15336348 G A); (15556 15337467 C T); (15557 15337486 C T); (15558 15337841 A C); (15559
15341924 A G); (15560 15343635 G A); (15561 15343793 G C); (15562 15345517 A G); (15563 15345637
G A); (15564 15345641 C T); (15565 15345876 C T); (15566 15346058 G A); (15567 15346406 C
A); (15568 15347257 C T); (15569 15347593 C T); (15570 15348643 A G); (15571 15348713 A G); (15572
15348815 A AT); (15573 15349058 A G); (15574 15349157 G T); (15575 15349465 A C); (15576
15349796 G T); (15577 15350207 A C); (15578 15351927 T A); (15579 15352471 G A); (15580 15352885
A G); (15581 15353490 T A); (15582 15354018 C A); (15583 15354038 C A); (15584 15354355 T
A); (15585 15358868 T C); (15586 15359370 G A); (15587 15360639 C CT); (15588 15360668 T G); (15589
15360717 T C); (15590 15360926 A AT); (15591 15361039 G A); (15592 15361928 A G); (15593
15361932 G A); (15594 15362208 C T); (15595 15362570 C T); (15596 15362632 A C); (15597 15364163
A G); (15598 15364804 T C); (15599 15366946 A G); (15600 15366950 A C); (15601 15368742 C
T); (15602 15369109 C T); (15603 15375544 C T); (15604 15376490 G A); (15605 15376803 A G); (15606
15379953 C T); (15607 15380014 A G); (15608 15381046 G A); (15609 15382305 CT C); (15610 15383017
C T); (15611 15383041 C T); (15612 15384728 AC A); (15613 15384926 C T); (15614 15386066 T
C); (15615 15386666 T TA); (15616 15387032 A T); (15617 15387138 T C); (15618 15388195 G T); (15619
15388435 C G); (15620 15390136 A G); (15621 15392385 C CGCGGGTCGCGCCGCACCCCAA); (15622
15394632 G A); (15623 15397003 A G); (15624 15397108 C T); (15625 15397380 C T); (15626 15404406
G C); (15627 15404710 C T); (15628 15405155 G A); (15629 15408672 C T); (15630 15409179 C T); (15631
15409186 T C); (15632 15411386 A C); (15633 15411994 G A); (15634 15420954 A C); (15635 15423294
G C); (15636 15424109 C T); (15637 15424393 G A); (15638 15424749 G A); (15639 15426436 T
C); (15640 15426797 A G); (15641 15426969 C T); (15642 15427189 A G); (15643 15427484 A T); (15644
15428319 T C); (15645 15428548 C A); (15646 15430558 C T); (15647 15434399 C T); (15648 15438885 A
AGAAACT); (15649 15439263 C T); (15650 15440347 G GAAAAATCGTCATCGTATGTT); (15651 15443525
A G); (15652 15444121 C T); (15653 15444781 C T); (15654 15444866 C T); (15655 15445070 C G); (15656
15445310 A G); (15657 15445634 G A); (15658 15445943 G A); (15659 15446348 T C); (15660 15447936
C T); (15661 15448170 C A); (15662 15449005 C T); (15663 15449304 G A); (15664 15450019 G
A); (15665 15450044 G A); (15666 15450177 A G); (15667 15451613 A G); (15668 15453241 T C); (15669
15453243 G T); (15670 15453247 A G); (15671 15453374 A AT); (15672 15455346 T C); (15673 15457342
T C); (15674 15457436 GTT G); (15675 15459860 C A); (15676 15460672 G A); (15677 15461899 T
C); (15678 15464970 G C); (15679 15466300 C T); (15680 15466858 T C); (15681 15471554 A C); (15682
15474562 A C); (15683 15474794 C A); (15684 15476789 C A); (15685 15477201 A C); (15686 15477468
C T); (15687 15478001 A C); (15688 15481340 C T); (15689 15481518 A C); (15690 15481887 T C); (15691
15482034 A G); (15692 15482263 A G); (15693 15482374 C T); (15694 15482463 AT A); (15695
15487193 A G); (15696 15488130 A G); (15697 15488250 C T); (15698 15488278 G A); (15699 15488860
A T); (15700 15489069 T C); (15701 15489088 A AT); (15702 15489229 A AT); (15703 15489658 T
TAG); (15704 15489865 C T); (15705 15489895 T C); (15706 15490236 A G); (15707 15491523 A
G); (15708 15491973 C T); (15709 15492216 T TA); (15710 15492739 TA T); (15711 15493263 C
T); (15712 15493303 G A); (15713 15493491 A G); (15714 15494078 G A); (15715 15494194 C T); (15716
15494262 G A); (15717 15494454 T G); (15718 15495014 A G); (15719 15499246 C CA); (15720
15499768 T C); (15721 15499950 C T); (15722 15500125 T C); (15723 15500462 A G); (15724 15503477
G A); (15725 15503610 G A); (15726 15505792 T A); (15727 15505796 TCAA T); (15728 15506941 A
C); (15729 15509635 C T); (15730 15512046 T G); (15731 15512386 C T); (15732 15512830 A G); (15733
15512989 A G); (15734 15512999 C A); (15735 15513708 A G); (15736 15513720 A T); (15737 15514168
G A); (15738 15514339 T G); (15739 15514466 A G); (15740 15517971 A T); (15741 15519590 T
C); (15742 15520324 T G); (15743 15520700 G A); (15744 15520890 T G); (15745 15520891 T A); (15746
15520892 G A); (15747 15521499 T C); (15748 15521681 G A); (15749 15521723 T C); (15750 15522250
C G); (15751 15522812 G A); (15752 15522844 T C); (15753 15523266 G GC); (15754 15523307 C
T); (15755 15525206 C A); (15756 15525279 A C); (15757 15527885 C T); (15758 15528135 C T); (15759
15529425 C T); (15760 15530625 T TGA); (15761 15530922 TA T); (15762 15531118 T A); (15763
15531262 G A); (15764 15535146 A C); (15765 15541352 C G); (15766 15543614 T A); (15767 15545479
T G); (15768 15549320 T G); (15769 15549485 C T); (15770 15551385 A G); (15771 15552945 G
A); (15772 15553858 G T); (15773 15553944 G T); (15774 15554007 G A); (15775 15554013 A G); (15776
15554068 G A); (15777 15554112 A T); (15778 15554115 G A); (15779 15554135 C T); (15780 15555455
C T); (15781 15555506 A G); (15782 15555853 A G); (15783 15555925 C T); (15784 15556559 G
A); (15785 15557535 T C); (15786 15558114 G A); (15787 15559283 A C); (15788 15560978 T C); (15789
15562918 G A); (15790 15563835 C A); (15791 15566196 C T); (15792 15567238 C T); (15793 15567280
A G); (15794 15567437 A G); (15795 15568213 A G); (15796 15568672 T C); (15797 15569392 A
AC); (15798 15569461 T C); (15799 15570710 G A); (15800 15570758 C T); (15801 15571188 G A); (15802
15571896 G A); (15803 15572759 C A); (15804 15572824 G A); (15805 15572825 A G); (15806 15574968
A G); (15807 15576131 T C); (15808 15576397 A G); (15809 15577232 C T); (15810 15579469 C
T); (15811 15582081 C T); (15812 15582089 A G); (15813 15582365 C T); (15814 15583312 TAGAG
T); (15815 15583434 G C); (15816 15584886 G A); (15817 15584889 A G); (15818 15586206 C T); (15819
15586799 A G); (15820 15586869 G T); (15821 15587624 A T); (15822 15588259 G T); (15823 15589974
G T); (15824 15590695 T C); (15825 15591182 C T); (15826 15591927 A G); (15827 15592019 T C); (15828
15592281 T C); (15829 15592497 CG C); (15830 15592527 G A); (15831 15592680 G A); (15832
15592769 C T); (15833 15594962 T C); (15834 15595597 A G); (15835 15596202 A G); (15836 15596322
A G); (15837 15596330 T G); (15838 15596375 G A); (15839 15597840 C A); (15840 15598511 G
T); (15841 15598512 A T); (15842 15598637 A G); (15843 15599352 T C); (15844 15599566 G C); (15845
15599615 G T); (15846 15599619 G A); (15847 15600490 T C); (15848 15606672 G A); (15849 15606674
G A); (15850 15606724 G A); (15851 15606866 G A); (15852 15608440 T C); (15853 15608673 A
T); (15854 15609466 A T); (15855 15609515 A G); (15856 15609917 C G); (15857 15610220 G T); (15858
15610709 GTCCT G); (15859 15611315 A G); (15860 15611912 C T); (15861 15614494 C T); (15862
15614720 AT A); (15863 15614792 TTA T); (15864 15615416 C A); (15865 15616222 G T); (15866
15616231 T C); (15867 15618111 C T); (15868 15618284 T C); (15869 15619667 G A); (15870 15620353 C
T); (15871 15620918 T G); (15872 15625445 A C); (15873 15625508 C T); (15874 15625681 G A); (15875
15628286 A G); (15876 15632260 C T); (15877 15632280 T A); (15878 15633161 A G); (15879 15633442
A G); (15880 15633916 G A); (15881 15634429 G A); (15882 15634464 T C); (15883 15634788 G
A); (15884 15634861 T C); (15885 15635699 GAAAAGAAGA G); (15886 15639148 C T); (15887 15640163
G C); (15888 15641075 G T); (15889 15642117 A T); (15890 15642273 G A); (15891 15642612 GT
G); (15892 15644258 T C); (15893 15644293 G A); (15894 15644369 C T); (15895 15645251 C T); (15896
15647391 A G); (15897 15648144 G GA); (15898 15648340 G C); (15899 15649802 A C); (15900
15651020 G A); (15901 15651062 C T); (15902 15651483 C T); (15903 15651809 A G); (15904 15652381
G A); (15905 15652607 T TG); (15906 15653511 C A); (15907 15655441 C T); (15908 15656481 G
T); (15909 15659380 G T); (15910 15659839 C T); (15911 15660562 A C); (15912 15660597 A T); (15913
15660758 TG T); (15914 15660980 G A); (15915 15662691 C T); (15916 15663717 G T); (15917 15664470
G A); (15918 15668425 G C); (15919 15670650 G A); (15920 15671312 G A); (15921 15674569 C
T); (15922 15674687 A G); (15923 15674692 G A); (15924 15678759 C T); (15925 15682156 G A); (15926
15684035 T A); (15927 15684515 T C); (15928 15686437 A T); (15929 15686839 C
CATGAATGGAATATGATTGTTGGTATGTGAA); (15930 15686888 T A); (15931 15689107 T G); (15932
15690657 C T); (15933 15691677 T C); (15934 15692493 G C); (15935 15693036 G A); (15936 15693542
G A); (15937 15694045 G A); (15938 15694521 T C); (15939 15694902 T A); (15940 15696137 C
A); (15941 15698002 C A); (15942 15699324 C T); (15943 15699414 T C); (15944 15699480 C T); (15945
15699625 C G); (15946 15699761 C T); (15947 15699925 C A); (15948 15701458 T C); (15949 15702566
C T); (15950 15702583 A G); (15951 15702932 A G); (15952 15702959 A C); (15953 15703205 A
AT); (15954 15703350 C A); (15955 15705029 T G); (15956 15705305 C T); (15957 15706578 C T); (15958
15706980 C T); (15959 15707068 T C); (15960 15707630 G A); (15961 15708449 C T); (15962 15710601 C
T); (15963 15710729 A G); (15964 15712549 G A); (15965 15712581 A G); (15966 15712797 A G); (15967
15714785 A G); (15968 15717528 G C); (15969 15720169 T C); (15970 15720234 G T); (15971 15722344
T C); (15972 15730756 C T); (15973 15733261 T A); (15974 15733707 T C); (15975 15739415 G A); (15976
15740540 T C); (15977 15740542 T C); (15978 15740865 T C); (15979 15741056 C T); (15980 15741222 C
CAG); (15981 15741965 T C); (15982 15741966 C T); (15983 15741967 A T); (15984 15742948 T
C); (15985 15743338 C T); (15986 15743598 G A); (15987 15744994 G T); (15988 15746905 G A); (15989
15747445 C A); (15990 15752196 C A); (15991 15753443 G A); (15992 15753450 C T); (15993 15753844
C T); (15994 15754538 G T); (15995 15754577 A C); (15996 15757219 C T); (15997 15758037 G
A); (15998 15758492 T C); (15999 15758498 C T); (16000 15758512 C T); (16001 15758538
TATATATATATATAC T); (16002 15759757 G GT); (16003 15760026 C CT); (16004 15760047 C T); (16005
15760054 C T); (16006 15760389 T G); (16007 15760857 GA G); (16008 15762676 C T); (16009 15765370
G A); (16010 15765512 A AT); (16011 15765670 G A); (16012 15765953 G T); (16013 15765999 C
T); (16014 15766241 C T); (16015 15766331 T A); (16016 15767011 C G); (16017 15767028 C T); (16018
15767683 A G); (16019 15767707 G A); (16020 15767882 A G); (16021 15769003 C T); (16022 15769044
T G); (16023 15774289 T A); (16024 15774292 T A); (16025 15774293 A T); (16026 15774325
AATATATAT A); (16027 15774402 TA T); (16028 15776148 A T); (16029 15777107 T G); (16030 15777545
A C); (16031 15780551 G A); (16032 15782609 C T); (16033 15783809 TAGTGTACACATTTG T); (16034
15788792 G C); (16035 15789603 T C); (16036 15789642 TG T); (16037 15791134 T C); (16038 15791910
C CATAT); (16039 15792460 C CACTCGCGCCAACCCGGCGCT); (16040 15794246 T C); (16041 15801487 G
GTGTTGAA); (16042 15807254 T G); (16043 15807315 T TA); (16044 15808019 C T); (16045 15808781 G
A); (16046 15809968 T A); (16047 15810578 C T); (16048 15812556 G T); (16049 15812649 G T); (16050
15816254 G A); (16051 15818802 C T); (16052 15819006 G C); (16053 15819820 A G); (16054 15822952
T C); (16055 15824131 GATT G); (16056 15824732 A G); (16057 15825849 C CA); (16058 15825999 G
T); (16059 15827444 C A); (16060 15827541 G A); (16061 15829028 G A); (16062 15829271 T G); (16063
15829830 G GACA); (16064 15833933 G A); (16065 15836004 C T); (16066 15838568 C CA); (16067
15839834 G T); (16068 15842524 A G); (16069 15842628 G C); (16070 15844418 C CT); (16071
15844862 A G); (16072 15845646 T G); (16073 15846037 C T); (16074 15847534 A G); (16075 15847969
G GTCTATCATAGTGT); (16076 15848067 C T); (16077 15850073 G T); (16078 15850636 C T); (16079
15850902 A C); (16080 15851018 A G); (16081 15852448 T C); (16082 15852858 A C); (16083 15853009
C T); (16084 15853857 A G); (16085 15854454 G A); (16086 15855799 G A); (16087 15856678 T
A); (16088 15857029 T C); (16089 15857287 G A); (16090 15858259 G A); (16091 15858538 T C); (16092
15858622 C T); (16093 15858726 G A); (16094 15864175 A AT); (16095 15865291 T C); (16096 15865662
AT A); (16097 15867923 T C); (16098 15869324 T C); (16099 15869868 C T); (16100 15870476 C
T); (16101 15872076 G T); (16102 15872629 A G); (16103 15874208 G C); (16104 15874282 C A); (16105
15874283 C T); (16106 15874402 G C); (16107 15876328 A G); (16108 15879198 A G); (16109 15880094
G A); (16110 15880295 A G); (16111 15880465 G A); (16112 15881139 T C); (16113 15881463 T
C); (16114 15882600 T G); (16115 15882814 T C); (16116 15883199 A G); (16117 15883240 G A); (16118
15883648 G A); (16119 15885168 T C); (16120 15885276 C A); (16121 15885403 C T); (16122 15885404
T A); (16123 15885405 A G); (16124 15885406 A G); (16125 15885420 GT G); (16126 15886724 A
G); (16127 15887069 C G); (16128 15887182 T G); (16129 15887897 TA T); (16130 15888021 A C); (16131
15889295 T A); (16132 15890744 A G); (16133 15890860 T G); (16134 15890891 A AAT); (16135
15893838 C T); (16136 15894013 C T); (16137 15895115 T C); (16138 15895130 TC T); (16139 15895180
A G); (16140 15895673 G A); (16141 15895709 A G); (16142 15895921 C T); (16143 15896119 CA
C); (16144 15897075 GT G); (16145 15897346 C T); (16146 15897626 G T); (16147 15898417 A T); (16148
15899033 T C); (16149 15902362 G GC); (16150 15902909 A C); (16151 15905659 C T); (16152 15905689
TTG); (16153 15905691 G T); (16154 15907907 A G); (16155 15907989 T A); (16156 15908053 C
T); (16157 15908070 G A); (16158 15908099 C T); (16159 15908108 A C); (16160 15908116 C T); (16161
15908124 T C); (16162 15908130 T C); (16163 15908131 T C); (16164 15908132 T C); (16165 15908685 A
AG); (16166 15909165 G A); (16167 15910764 AATGTAC A); (16168 15911137 G A); (16169 15912061 G
T); (16170 15913382 T C); (16171 15916155 C T); (16172 15916650 C A); (16173 15917275 CT C); (16174
15917586 A AT); (16175 15917833 A G); (16176 15918237 T C); (16177 15918662 T C); (16178 15918746
G A); (16179 15919467 C CT); (16180 15921256 C T); (16181 15921394 TTATA T); (16182 15922683 C
G); (16183 15923669 G GA); (16184 15925387 G A); (16185 15927815 T G); (16186 15930731 C
G); (16187 15930762 C A); (16188 15932133 A T); (16189 15932865 C T); (16190 15934232 C T); (16191
15934298 A T); (16192 15936015 C T); (16193 15938015 T G); (16194 15938543 C A); (16195 15938852
A T); (16196 15939085 T C); (16197 15941894 A T); (16198 15942049 T C); (16199 15943849 C T); (16200
15943861 T C); (16201 15946397 T C); (16202 15946682 CCTT C); (16203 15947164 G A); (16204
15949390 C A); (16205 15950106 A G); (16206 15950630 T C); (16207 15950657 G A); (16208 15951198
A C); (16209 15956081 CA C); (16210 15956969 G A); (16211 15960188 GTGTATGTATGTA G); (16212
15961607 A G); (16213 15961664 G A); (16214 15961783 C G); (16215 15962903 A G); (16216 15962994
A AT); (16217 15964364 T TA); (16218 15964719 T C); (16219 15965050 T C); (16220 15965120 G
A); (16221 15965412 G C); (16222 15966001 T C); (16223 15969850 C T); (16224 15969882 G A); (16225
15970425 T C); (16226 15972604 G A); (16227 15972666 T G); (16228 15973574 C T); (16229 15975626
T C); (16230 15978585 T TTA); (16231 15979662 C T); (16232 15980047 C T); (16233 15982897 T
C); (16234 15983616 G A); (16235 15984721 A C); (16236 15985398 T C); (16237 15989075 G A); (16238
15991359 C T); (16239 15992152 T A); (16240 15992205 G A); (16241 15996588 C T); (16242 15997408
A T); (16243 16000362 A G); (16244 16001029 C T); (16245 16001460 C T); (16246 16001962 A
G); (16247 16002281 G T); (16248 16004597 T C); (16249 16004629 T C); (16250 16005930 G A); (16251
16006088 C T); (16252 16008723 CAACTT C); (16253 16010076 G A); (16254 16011206 C T); (16255
16012157 G A); (16256 16012929 T C); (16257 16012998 G A); (16258 16014660 G A); (16259 16017459
T C); (16260 16017528 G A); (16261 16019106 C T); (16262 16019216 C T); (16263 16019484 C T); (16264
16021425 G A); (16265 16021478 T C); (16266 16025547 G GAAAACAAACAAAAAATAT); (16267
16025607 C T); (16268 16026280 C T); (16269 16029272 C T); (16270 16032457 G T); (16271 16034210
GT G); (16272 16034240 G A); (16273 16034585 C T); (16274 16037605 C A); (16275 16040421 C
T); (16276 16040519 AGTGAT A); (16277 16041985 G A); (16278 16042357 T G); (16279 16043601 T
TTTATTATTATTATTATTATTA); (16280 16046183 T A); (16281 16046357 TC T); (16282 16046359 T
A); (16283 16046380 C T); (16284 16046386 C T); (16285 16046510 C T); (16286 16046637 T C); (16287
16047694 GA G); (16288 16051342 T G); (16289 16051480 CTACAAAAGGCA C); (16290 16051558 A
C); (16291 16052870 C T); (16292 16055005 C T); (16293 16055643 T C); (16294 16058039 C A); (16295
16058907 G A); (16296 16063641 C T); (16297 16063724 G A); (16298 16068524 T C); (16299 16071483
CCGGGGGAGTGGTG C); (16300 16072824 T TTGGCGCTGA); (16301 16073244 T TA); (16302 16073999 G
A); (16303 16074089 G A); (16304 16074397 C T); (16305 16075154 A
ATGGCGCCATCTGGCGCTAC); (16306 16077050 C G); (16307 16079942 T G); (16308 16080036 C
CATAT); (16309 16080725 G T); (16310 16082041 T C); (16311 16083199 T C); (16312 16083576 T
C); (16313 16083792 G GT); (16314 16083866 T A); (16315 16084024 A G); (16316 16084113 A
G); (16317 16089073 A AAGTGAAGGTACTTTATACCAAATT); (16318 16089083 T C); (16319 16089426 T
C); (16320 16089625 CTTA C); (16321 16091363 T A); (16322 16093233 G A); (16323 16093262 A
G); (16324 16093330 C G); (16325 16093391 A G); (16326 16093407 A G); (16327 16093617 G
GA); (16328 16095825 T C); (16329 16097972 T C); (16330 16098007 C T); (16331 16098760 T C); (16332
16101880 C T); (16333 16102767 C G); (16334 16107011 T C); (16335 16107318 C T); (16336 16108223 T
C); (16337 16108524 G A); (16338 16108534 A G); (16339 16112739 CATCCA C); (16340 16112834 C
T); (16341 16113037 G A); (16342 16114442 T C); (16343 16115202 A G); (16344 16115390 G A); (16345
16115526 T C); (16346 16115970 A G); (16347 16118069 G C); (16348 16118094 C A); (16349 16118182
C T); (16350 16119139 C T); (16351 16119413 A G); (16352 16119470 C T); (16353 16119639 G
A); (16354 16120166 C T); (16355 16125327 G T); (16356 16125360 T C); (16357 16126086 A C); (16358
16126159 T C); (16359 16128403 GA G); (16360 16129391 C A); (16361 16131071 A C); (16362
16131271 AT A); (16363 16132899 A T); (16364 16132989 T C); (16365 16133111 A C); (16366 16133265
G T); (16367 16135215 G GCGCTAATAGCGTGGATCCTCA); (16368 16135991 A C); (16369 16136237 C
G); (16370 16137816 G C); (16371 16138238 C T); (16372 16138508 G A); (16373 16140493 C T); (16374
16144021 G A); (16375 16145979 C T); (16376 16146657 A G); (16377 16146661 G A); (16378 16146731
G A); (16379 16146772 GA G); (16380 16146854 G A); (16381 16146874 G T); (16382 16147210 G
C); (16383 16147270 A G); (16384 16148175 A G); (16385 16148497 G A); (16386 16149678 G T); (16387
16149826 G A); (16388 16151091 C T); (16389 16151092 A C); (16390 16151826 C A); (16391 16152492
A C); (16392 16153217 G A); (16393 16155127 G T); (16394 16155385 C T); (16395 16155635 G
GTT); (16396 16155682 C T); (16397 16156885 T C); (16398 16160468 C T); (16399 16160566 G
GAA); (16400 16161124 A AT); (16401 16161441 A G); (16402 16161661 G A); (16403 16162232 A
C); (16404 16163239 G T); (16405 16163889 C T); (16406 16165895 T C); (16407 16167478 T C); (16408
16171249 C T); (16409 16172121 C T); (16410 16174973 A ACTAAACCCTAAACC); (16411 16177568 A
G); (16412 16178347 A C); (16413 16178461 G A); (16414 16178697 T C); (16415 16179046 G A); (16416
16185152 C T); (16417 16186261 G C); (16418 16186315 G A); (16419 16186971 A G); (16420 16187281
A T); (16421 16189559 A C); (16422 16189560 G A); (16423 16189581 G T); (16424 16189582 A
G); (16425 16191352 G A); (16426 16193127 A T); (16427 16193167 C G); (16428 16195463 A G); (16429
16195584 G A); (16430 16198680 T C); (16431 16198797 T C); (16432 16201051 G A); (16433 16201448
T C); (16434 16202917 G A); (16435 16206673 C T); (16436 16208589 G A); (16437 16208897 G
A); (16438 16208947 A G); (16439 16210119 T C); (16440 16210121 A G); (16441 16210273 A G); (16442
16211463 C G); (16443 16211781 C T); (16444 16212508 C T); (16445 16212842 G GA); (16446
16212952 T A); (16447 16212979 T A); (16448 16215418 C T); (16449 16215764 T A); (16450 16217188 A
G); (16451 16217189 T G); (16452 16217190 A T); (16453 16217191 T A); (16454 16217192 A C); (16455
16217193 T G); (16456 16217195 T A); (16457 16217930 G A); (16458 16218875 TATAC T); (16459
16219053 A AT); (16460 16220128 A G); (16461 16221415 C T); (16462 16222217 G A); (16463
16223625 C T); (16464 16225184 A C); (16465 16228746 A G); (16466 16229926 A G); (16467 16230210
GA G); (16468 16230904 A G); (16469 16231151 T C); (16470 16231169 G A); (16471 16231387 A
G); (16472 16234224 T TTA); (16473 16235161 C T); (16474 16235312 G GA); (16475 16235390 CT
C); (16476 16236676 A G); (16477 16236776 T C); (16478 16238251 G A); (16479 16243779 T G); (16480
16243798 T G); (16481 16244293 C T); (16482 16244345 G A); (16483 16244658 GA G); (16484
16245899 A G); (16485 16246249 A G); (16486 16250022 A G); (16487 16250434 A C); (16488 16251322
A G); (16489 16252492 A G); (16490 16252662 A G); (16491 16253918 C T); (16492 16256668 T
C); (16493 16257550 G A); (16494 16258105 A C); (16495 16259054 C A); (16496 16259635 G T); (16497
16260566 G A); (16498 16261521 A G); (16499 16262113 C T); (16500 16262609 A G); (16501 16263280
C T); (16502 16264102 T C); (16503 16265453 C G); (16504 16265558 G A); (16505 16266592 AT
A); (16506 16267854 C T); (16507 16268102 T C); (16508 16269868 T C); (16509 16269911 T A); (16510
16272276 C T); (16511 16272517 A C); (16512 16274125 G A); (16513 16278835 G C); (16514 16278937
A G); (16515 16283843 A ACATAT); (16516 16283886 T G); (16517 16285658 C T); (16518 16285790 C
T); (16519 16285866 A G); (16520 16285957 A G); (16521 16287122 G A); (16522 16287376 T C); (16523
16288989 G A); (16524 16289003 G A); (16525 16289363 G C); (16526 16290811 T TA); (16527
16291429 G A); (16528 16292001 A G); (16529 16292067 C T); (16530 16292880 A G); (16531 16294270
G C); (16532 16295061 G A); (16533 16296337 G A); (16534 16297175 G A); (16535 16299089 A
G); (16536 16299580 T C); (16537 16300120 T G); (16538 16305263 G A); (16539 16307079 A T); (16540
16307080 G T); (16541 16307081 G A); (16542 16307082 T A); (16543 16307105
ACCAAGAGGGGGGGTGAATTGGT A); (16544 16308424 T G); (16545 16308851 G A); (16546 16309314 A
G); (16547 16309327 A C); (16548 16312476 G A); (16549 16313467 C T); (16550 16317846 G A); (16551
16320244 T A); (16552 16320497 AT A); (16553 16322032 C A); (16554 16322375 A G); (16555
16322753 C T); (16556 16324505 T G); (16557 16325777 A G); (16558 16326261 T G); (16559 16326339
C T); (16560 16327097 C T); (16561 16327463 A G); (16562 16328984 T A); (16563 16329073 A
G); (16564 16329495 T C); (16565 16331288 G T); (16566 16331492 G T); (16567 16334399 G A); (16568
16334738 C T); (16569 16334997 A G); (16570 16335319 A ATAATT); (16571 16336241 C T); (16572
16337151 C CT); (16573 16339047 G A); (16574 16339186 C A); (16575 16340060 T G); (16576 16340319
C T); (16577 16340818 T C); (16578 16340833 T TGG); (16579 16341093 C T); (16580 16341096 C
T); (16581 16341231 C T); (16582 16341429 T C); (16583 16342129 G GA); (16584 16346434 A G); (16585
16348127 AT A); (16586 16348600 A AT); (16587 16349599 C A); (16588 16351432 A G); (16589
16352201 G A); (16590 16352309 G C); (16591 16353034 C A); (16592 16355575 A T); (16593 16356287
T G); (16594 16364335 A G); (16595 16364348 G A); (16596 16364631 T C); (16597 16367534 T
C); (16598 16367708 GA G); (16599 16368191 C A); (16600 16368378 G GT); (16601 16369607 C
G); (16602 16370096 C T); (16603 16371386 G A); (16604 16376459 C T); (16605 16376944 A G); (16606
16378235 T C); (16607 16379760 C T); (16608 16381618 G A); (16609 16381814 C G); (16610 16382603
CA C); (16611 16382627 T C); (16612 16384027 G A); (16613 16384243 G A); (16614 16384244 T
G); (16615 16384347 G A); (16616 16384354 T C); (16617 16385240 C A); (16618 16385442 T C); (16619
16385477 G A); (16620 16389509 G A); (16621 16390208 C T); (16622 16391096 A G); (16623 16395488
G A); (16624 16396165 T TCGCGCCACAGTGGCGCCATG); (16625 16399574 G A); (16626 16400726 G
C); (16627 16400844 G A); (16628 16401009 TA T); (16629 16401114 A G); (16630 16401332 A C); (16631
16404515 T TATATAG); (16632 16409749 T G); (16633 16409842 C T); (16634 16409962 T C); (16635
16409978 T A); (16636 16410216 A G); (16637 16410233 A G); (16638 16411063 T C); (16639 16411086
T C); (16640 16411126 C T); (16641 16411138 C T); (16642 16411167 G C); (16643 16412810 G T); (16644
16414626 G A); (16645 16415063 G A); (16646 16417000 C T); (16647 16418372 G T); (16648 16421130
G A); (16649 16422893 C T); (16650 16423037 G C); (16651 16424633 A AC); (16652 16425195 A
G); (16653 16425229 C T); (16654 16425260 A AT); (16655 16427934 C T); (16656 16428052 A G); (16657
16428088 C A); (16658 16428586 A G); (16659 16428719 T C); (16660 16428736 C A); (16661 16429089
A G); (16662 16429098 A T); (16663 16429102 G A); (16664 16433222 A G); (16665 16434939 G
A); (16666 16435066 CAGCATTT C); (16667 16435067 G A); (16668 16436223 T TTTTTCTTC); (16669
16436870 C T); (16670 16437169 G C); (16671 16437604 C A); (16672 16437825 C T); (16673 16439243
G T); (16674 16442993 A G); (16675 16445254 T G); (16676 16445338 T C); (16677 16447231 T
C); (16678 16447446 A C); (16679 16450508 C T); (16680 16451710 C T); (16681 16451868 G A); (16682
16452886 G A); (16683 16453417 G A); (16684 16454050 C T); (16685 16454860 T C); (16686 16455385
C T); (16687 16455404 A C); (16688 16455874 A C); (16689 16456138 A G); (16690 16457522 A
G); (16691 16459059 C T); (16692 16460386 A G); (16693 16460625 G A); (16694 16460681 C T); (16695
16461122 G A); (16696 16461962 G A); (16697 16462027 C T); (16698 16463473 A G); (16699 16463585
G A); (16700 16464140 G A); (16701 16464481 G A); (16702 16465142 G A); (16703 16465555 G
A); (16704 16465704 C T); (16705 16465720 C A); (16706 16466600 G A); (16707 16467575 G A); (16708
16468280 C T); (16709 16468740 C T); (16710 16470254 T C); (16711 16470258 A T); (16712 16471124 G
A); (16713 16471258 A G); (16714 16471378 AT A); (16715 16476745 T C); (16716 16477830 C A); (16717
16478797 AT A); (16718 16479546 G A); (16719 16479807 G A); (16720 16481650 T C); (16721
16482896 G T)

TABLE 2
List of SNPs derived from crossing of Resistant male with susceptible female 1. SNPs listed in format SNP ID,
SNP position relative to reference genome, Favorable allele, Unfavorable allele, each separated by a space.

(1 9280996 A C); (2 9281013 G A); (3 9281195 C T); (4 9281200 A T); (5 9281201 C A); (6 9281223 C T); (7
9281276 A G); (8 9281393 C T); (9 9281485 C T); (10 9281647 G A); (11 9281724 AC A); (12 9281784 A
C); (13 9281826 C T); (14 9281835 G A); (15 9281849 G A); (16 9282235 C T); (17 9283196 G C); (18
9283409 A G); (19 9283754 T C); (20 9283852 T C); (21 9284242 A G); (22 9284284 T A); (23 9284396 C
T); (24 9284444 A G); (25 9284517 A T); (26 9284544 A T); (27 9284917 CT C); (28 9285271 A G); (29
9285329 T C); (30 9285641 C A); (31 9285701 A G); (32 9286741 G C); (33 9287126 G A); (34 9287346 T
G); (35 9287696 T G); (36 9287730 G A); (37 9288083 T A); (38 9288197 C T); (39 9288204 G GA); (40
9288360 T C); (41 9288370 G A); (42 9288405 G A); (43 9288427 T C); (44 9288663 G T); (45 9288686 T
C); (46 9288939 T C); (47 9289409 A G); (48 9289584 T C); (49 9290113 A G); (50 9290269 A G); (51
9290321 A T); (52 9290460 C T); (53 9290465 C T); (54 9290483 T C); (55 9290553 T C); (56 9290730 T
G); (57 9290769 A G); (58 9291038 G A); (59 9291072 T A); (60 9291087 C T); (61 9291179 TG T); (62
9291229 G T); (63 9291251 G A); (64 9291357 A T); (65 9291583 C G); (66 9291658 G A); (67 9291766 A
AT); (68 9291902 T C); (69 9291967 C T); (70 9292058 A AAT); (71 9292150 T C); (72 9292231
ATATGCTGAAAGTTTTT A); (73 9292418 C T); (74 9292437 C G); (75 9292503 C T); (76 9292772 G A); (77
9292792 G A); (78 9292832 C A); (79 9292848 G A); (80 9292864 A G); (81 9292865 G T); (82 9292866 C
T); (83 9293079 G A); (84 9293132 C T); (85 9293138 G A); (86 9293190 T G); (87 9293329 T C); (88
9293351 T A); (89 9293521 C T); (90 9293588 C T); (91 9293603 A C); (92 9293674 T C); (93 9293699 G
A); (94 9293925 C T); (95 9294113 G C); (96 9294157 C A); (97 9294234 A G); (98 9294289 T C); (99
9294332 T A); (100 9294612 G T); (101 9294776 C T); (102 9294805 G T); (103 9294966 GTA G); (104
9295411 G A); (105 9295472 C T); (106 9295596 T G); (107 9295618 A C); (108 9295751 T C); (109
9295841 G GA); (110 9295937 A T); (111 9296384 T C); (112 9296404 T A); (113 9296475 C A); (114
9296487 T C); (115 9296637 A AT); (116 9296728 T A); (117 9296787 T C); (118 9296822 T A); (119
9296823 A T); (120 9297052 C A); (121 9297264 C G); (122 9297354 A G); (123 9297425 C T); (124
9297796 T C); (125 9297798 T C); (126 9297884 C T); (127 9297889 C T); (128 9297939 T C); (129
9298007 T G); (130 9298047 G A); (131 9298211 T A); (132 9298262 C CG); (133 9298394 T C); (134
9298603 G GT); (135 9298626 C A); (136 9298708 A G); (137 9298754 G A); (138 9298845 C T); (139
9298846 T C); (140 9298946 A T); (141 9299120 G T); (142 9299177 C T); (143 9299209 T G); (144
9299247 G A); (145 9299327 C T); (146 9299776 C A); (147 9300207 A G); (148 9300267 G T); (149
9300355 A G); (150 9300405 G T); (151 9301308 A AT); (152 9301458 A G); (153 9301541 CT C); (154
9301543 A G); (155 9301674 G A); (156 9301769 G A); (157 9302189 G A); (158 9302192 A G); (159
9302333 T TG); (160 9302588 A G); (161 9302849 T C); (162 9302877 G C); (163 9302948 A G); (164
9302955 G C); (165 9303130 C G); (166 9303367 T C); (167 9303439 A G); (168 9303535 C T); (169
9303598 C T); (170 9304281 G A); (171 9304590 A G); (172 9304844 C T); (173 9304928 A G); (174
9304937 A G); (175 9304965 C G); (176 9305374 C T); (177 9305697 G A); (178 9305841 C T); (179
9305944 C T); (180 9305950 C G); (181 9306100 A C); (182 9306484 A G); (183 9307000 C T); (184
9307076 A G); (185 9307127 T C); (186 9307261 G A); (187 9307291 A C); (188 9307379 C A); (189
9307667 G A); (190 9307730 T C); (191 9308097 G C); (192 9308169 G A); (193 9308353 C T); (194
9308594 T C); (195 9308952 T C); (196 9309199 A G); (197 9309420 T C); (198 9309498 C T); (199
9309860 T G); (200 9309950 AT A); (201 9310002 T C); (202 9310042 T C); (203 9310099 T C); (204
9310151 T C); (205 9310299 A T); (206 9311612 T A); (207 9311642 C T); (208 9311656 G A); (209
9311677 C T); (210 9311734 T C); (211 9311754 G A); (212 9311759 G A); (213 9311762 TG T); (214
9311835 C T); (215 9311873 G T); (216 9312082 G A); (217 9312273 G A); (218 9312427 T C); (219
9312529 G A); (220 9312575 T C); (221 9312591 G A); (222 9312701 T A); (223 9312834 C G); (224
9312872 G A); (225 9312944 C A); (226 9313018 T C); (227 9313095 G A); (228 9313213 C A); (229
9313398 C T); (230 9314094 C T); (231 9314218 C A); (232 9314770 A G); (233 9314884 T C); (234
9314924 G T); (235 9315263 A G); (236 9315836 T C); (237 9315999 T G); (238 9316000 A G); (239
9316145 A G); (240 9316189 A G); (241 9316195 C T); (242 9316203 A G); (243 9316326 A G); (244
9316493 C T); (245 9316703 A G); (246 9316769 G A); (247 9317531 C A); (248 9317571 G A); (249
9317606 G T); (250 9317801 G T); (251 9317954 T C); (252 9318210 G A); (253 9318370 T C); (254
9318500 A G); (255 9318582 G A); (256 9318584 G A); (257 9318599 T
TTCTAGAAATGTGATGATAATTGGA); (258 9318682 T A); (259 9319112 C G); (260 9319821 A G); (261
9319878 T A); (262 9320091 G T); (263 9320107 TTATA T); (264 9320144 T G); (265 9320368 A AT); (266
9320434 T C); (267 9320488 G A); (268 9320514 C T); (269 9320550 T A); (270 9320790 GA G); (271
9321074 T C); (272 9321284 T C); (273 9321369 G T); (274 9321439 G A); (275 9321706 A T); (276
9321900 G A); (277 9322060 GT G); (278 9322073 C A); (279 9322310 A C); (280 9322361 A C); (281
9322434 T C); (282 9322505 T C); (283 9322662 A G); (284 9322667 C T); (285 9322860 G T); (286
9322981 A G); (287 9323008 T C); (288 9323921 G A); (289 9324043 A G); (290 9324285 T C); (291
9324748 A C); (292 9324814 T C); (293 9325202 C T); (294 9325253 A G); (295 9325415 A C); (296
9325636 T C); (297 9326516 G A); (298 9326533 C T); (299 9326743 G T); (300 9326764 A G); (301
9326857 T A); (302 9327012 A G); (303 9328391 T C); (304 9328946 T C); (305 9329079 C CT); (306
9329095 C T); (307 9329477 T G); (308 9329522 C T); (309 9329916 T C); (310 9330583 T C); (311
9330685 A G); (312 9331019 T C); (313 9331289 C T); (314 9331960 G A); (315 9332360 T C); (316
9332383 A G); (317 9332469 A C); (318 9333204 G A); (319 9333391 A AT); (320 9333539 C G); (321
9333657 G A); (322 9334350 T C); (323 9334538 A ATT); (324 9334549 A AT); (325 9334784 T C); (326
9334873 G A); (327 9334946 A G); (328 9335025 C T); (329 9335192 C G); (330 9335202 A G); (331
9335588 TA T); (332 9335684 T G); (333 9335750 C A); (334 9335859 A G); (335 9336658 A G); (336
9336772 A G); (337 9336799 AC A); (338 9336881 C T); (339 9336930 T C); (340 9337090 T A); (341
9337643 A G); (342 9337827 C A); (343 9337957 T A); (344 9338549 C T); (345 9338802 A G); (346
9339150 C T); (347 9339593 C T); (348 9339699 C T); (349 9339768 G A); (350 9339829 T C); (351
9339971 G T); (352 9340302 G A); (353 9340326 T C); (354 9340921 A G); (355 9341245 G A); (356
9341294 G A); (357 9341328 G A); (358 9341482 T C); (359 9341768 G A); (360 9341981 C T); (361
9342009 C T); (362 9342188 C T); (363 9342315 G A); (364 9342331 G T); (365 9342460 G
GAAGGGA); (366 9342704 G GAAGT); (367 9342863 C G); (368 9342865 G A); (369 9342979 C T); (370
9343228 G T); (371 9343447 C T); (372 9343585 G A); (373 9343615 T C); (374 9343690 C CA); (375
9343735 A G); (376 9344153 C T); (377 9344373 G A); (378 9344431 T C); (379 9344444 A G); (380
9344866 C T); (381 9344979 G A); (382 9345231 A G); (383 9345406 C T); (384 9345511 A G); (385
9345659 G A); (386 9345691 G A); (387 9345772 C G); (388 9345959 C T); (389 9346602 C T); (390
9346657 G A); (391 9346848 A G); (392 9346895 A G); (393 9346932 G A); (394 9346956 A T); (395
9347477 C A); (396 9347479 A G); (397 9347538 T C); (398 9347742 C A); (399 9348068 A G); (400
9348125 A G); (401 9348128 G T); (402 9348348 T A); (403 9348652 A G); (404 9348762 C T); (405
9348891 C T); (406 9348949 C T); (407 9349015 A G); (408 9349036 G C); (409 9349106 G A); (410
9349497 T C); (411 9349558 G T); (412 9349625 T C); (413 9349661 T G); (414 9349953 G A); (415
9350269 A G); (416 9350351 C T); (417 9350464 C T); (418 9350510 C T); (419 9350533 G A); (420
9350573 T C); (421 9350648 C T); (422 9350653 T C); (423 9350802 A G); (424 9350827 C T); (425
9350938 T G); (426 9350952 G A); (427 9351006 A G); (428 9351047 C T); (429 9351212 C T); (430
9351265 C T); (431 9351282 T A); (432 9351396 A G); (433 9351628 C T); (434 9351657 T C); (435
9352012 C T); (436 9352417 A G); (437 9352426 G A); (438 9352496 C T); (439 9352590 A G); (440
9352648 C T); (441 9353102 T C); (442 9353385 G A); (443 9353642 C T); (444 9353739 T C); (445
9355287 A G); (446 9355377 C T); (447 9355470 A G); (448 9355580 T C); (449 9355723 C G); (450
9355886 T C); (451 9356166 C T); (452 9356258 C T); (453 9356410 G T); (454 9356506 C T); (455
9356880 C G); (456 9357107 T A); (457 9357432 G T); (458 9357664 T C); (459 9357775 T C); (460
9358155 G A); (461 9358238 C T); (462 9358254 G A); (463 9358298 A C); (464 9358306 G A); (465
9358395 T A); (466 9358518 C T); (467 9358636 C T); (468 9358712 C A); (469 9358713 G A); (470
9358729 A G); (471 9358746 T C); (472 9359091 T G); (473 9359423 A G); (474 9359564 C A); (475
9359590 G A); (476 9360107 T C); (477 9360188 A C); (478 9360295 C T); (479 9360391 C G); (480
9360596 C T); (481 9360747 C CT); (482 9360963 T A); (483 9361075 C T); (484 9361086 G A); (485
9361165 G A); (486 9361743 A C); (487 9362056 G A); (488 9362193 T C); (489 9362542 A G); (490
9362836 T C); (491 9363196 G A); (492 9363541 T G); (493 9363770 T C); (494 9363799 A G); (495
9363826 A G); (496 9363954 A G); (497 9364160 G A); (498 9364338 A G); (499 9364393 G T); (500
9364530 G A); (501 9364690 C T); (502 9364714 A T); (503 9364728 TGA T); (504 9364946 AT A); (505
9365048 C G); (506 9365069 T C); (507 9365105 A G); (508 9365170 C T); (509 9365282 C T); (510
9365422 T C); (511 9365761 A C); (512 9366289 G C); (513 9366787 G T); (514 9366839 T G); (515
9367003 G A); (516 9367058 A T); (517 9367667 A T); (518 9367714 A T); (519 9369144 G T); (520
9369165 C A); (521 9369460 A C); (522 9369605 G A); (523 9369903 T A); (524 9370187 G C); (525
9370749 G A); (526 9370846 T A); (527 9370861 G A); (528 9371177 G A); (529 9371196 A G); (530
9371325 ATGAG A); (531 9371417 G T); (532 9371716 A G); (533 9371982 C A); (534 9372737 T G); (535
9373077 T C); (536 9373180 C T); (537 9373436 C T); (538 9373843 G A); (539 9374000 G A); (540
9374011 C T); (541 9374275 G A); (542 9374490 T C); (543 9374546 C T); (544 9374876 G A); (545
9374965 C T); (546 9375058 G A); (547 9375114 C T); (548 9375188 A G); (549 9375650 C T); (550
9376035 G A); (551 9376099 G A); (552 9376111 T C); (553 9376116 A G); (554 9376317 G A); (555
9376326 G A); (556 9376391 A G); (557 9376550 T C); (558 9376728 C T); (559 9377202 A G); (560
9378067 T C); (561 9378123 A T); (562 9378490 A C); (563 9378539 G A); (564 9378653 G A); (565
9378687 A G); (566 9378758 C T); (567 9378819 T C); (568 9378851 T C); (569 9378939 T C); (570
9379081 C T); (571 9379184 G A); (572 9379317 G A); (573 9379399 G A); (574 9379407 T C); (575
9379438 A G); (576 9379445 A G); (577 9379474 G T); (578 9379975 A G); (579 9380272 G A); (580
9380686 T C); (581 9380880 A G); (582 9381320 T C); (583 9381609 A C); (584 9381727 C T); (585
9381867 C T); (586 9381873 C T); (587 9381933 G A); (588 9382008 C T); (589 9382644 T C); (590
9382645 G A); (591 9382891 G T); (592 9382962 T C); (593 9383009 T C); (594 9383038 G A); (595
9383658 T G); (596 9383684 G A); (597 9383718 A T); (598 9383897 A T); (599 9383967 T G); (600
9384157 C T); (601 9384207 C T); (602 9384432 C T); (603 9385042 G A); (604 9385174 A G); (605
9385394 A G); (606 9385471 A G); (607 9385567 C T); (608 9385680 A G); (609 9386021 T C); (610
9386252 A T); (611 9386314 C A); (612 9386338 G T); (613 9386356 C T); (614 9386382 G A); (615
9386385 G C); (616 9386412 A G); (617 9386578 G A); (618 9386599 C T); (619 9387042 T C); (620
9387067 C T); (621 9388241 C T); (622 9389115 G A); (623 9389510 G T); (624 9389584 G C); (625
9389646 C G); (626 9390011 C T); (627 9390035 T C); (628 9390233 T C); (629 9390454 G T); (630
9390522 T C); (631 9390578 G A); (632 9390643 T C); (633 9390826 C T); (634 9390965 A T); (635
9391244 T C); (636 9391287 C A); (637 9391414 A G); (638 9391671 G A); (639 9392428 A G); (640
9392577 A G); (641 9392774 T C); (642 9393285 G A); (643 9393297 A C); (644 9393426 C A); (645
9393551 T C); (646 9393559 G A); (647 9393628 G C); (648 9394563 A T); (649 9394624 A G); (650
9394954 A T); (651 9395187 C G); (652 9395688 G C); (653 9395755 T TA); (654 9396073 C T); (655
9396508 G T); (656 9397311 T A); (657 9397514 A G); (658 9398486 GA G); (659 9399145 G GA); (660
9399295 G A); (661 9399316 G A); (662 9399522 A G); (663 9399851 G T); (664 9400266 C A); (665
9400291 T A); (666 9400974 G A); (667 9401122 A T); (668 9401154 A G); (669 9401170 T
TTAATTAAAAAAACA); (670 9401282 T C); (671 9401457 C T); (672 9401508 C T); (673 9401600 A G); (674
9402229 C A); (675 9402238 G C); (676 9402255 C T); (677 9402833 T C); (678 9402872 G T); (679
9402948 T C); (680 9402998 G A); (681 9403120 T C); (682 9403147 G C); (683 9403580 G GTA); (684
9403622 A T); (685 9403658 G A); (686 9403684 T A); (687 9403729 G A); (688 9403741 C T); (689
9403825 G A); (690 9404163 G A); (691 9404375 T G); (692 9404395 G T); (693 9404448 G A); (694
9404661 A G); (695 9404713 G A); (696 9404743 C T); (697 9404759 G A); (698 9405051 T C); (699
9405081 C T); (700 9405407 G A); (701 9405413 C T); (702 9405419 A G); (703 9405421 A G); (704
9405581 A G); (705 9405685 T C); (706 9405764 G A); (707 9405780 GA G); (708 9405814 C T); (709
9405831 G A); (710 9405913 G C); (711 9406063 T C); (712 9406308 G A); (713 9406313 T C); (714
9406349 G A); (715 9406407 A G); (716 9406410 G A); (717 9406627 G A); (718 9406851 A G); (719
9406884 A G); (720 9406950 C T); (721 9406970 C T); (722 9407001 G A); (723 9407152 C T); (724
9407396 C T); (725 9407674 T G); (726 9407799 C G); (727 9407884 C T); (728 9407906 C T); (729
9407938 G A); (730 9408020 T A); (731 9408200 A G); (732 9408202 C T); (733 9408430 C T); (734
9408462 G C); (735 9408753 A G); (736 9409280 C T); (737 9409378 G T); (738 9409400 A C); (739
9409403 C T); (740 9409411 T G); (741 9409708 T C); (742 9409766 T C); (743 9409991 G A); (744
9410020 T TA); (745 9410119 G A); (746 9410144 T C); (747 9410162 T A); (748 9410374 A G); (749
9410477 C T); (750 9410503 C G); (751 9410620 C T); (752 9410744 G A); (753 9410766 G A); (754
9410871 A G); (755 9410922 T C); (756 9411050 A G); (757 9411131 G A); (758 9411147 T C); (759
9411168 C A); (760 9411310 G A); (761 9411366 T C); (762 9412124 C CTA); (763 9412169 T A); (764
9412530 C T); (765 9412678 T A); (766 9413038 T C); (767 9413065 A G); (768 9413123 G GT); (769
9413217 C T); (770 9413301 C A); (771 9413641 T C); (772 9413717 T C); (773 9413721 G A); (774
9413803 T C); (775 9414232 A G); (776 9414401 T C); (777 9414405 C T); (778 9414635 A G); (779
9414718 T C); (780 9414770 C T); (781 9414772 T C); (782 9414843 G A); (783 9414999 A G); (784
9415349 T C); (785 9415377 T C); (786 9415586 AAC A); (787 9415957 A G); (788 9416306 C G); (789
9416427 G A); (790 9416569 T C); (791 9416646 C T); (792 9416652 T C); (793 9416825 C T); (794
9416831 C A); (795 9416899 G C); (796 9416938 C T); (797 9417010 T C); (798 9417012 A G); (799
9417122 A G); (800 9417354 C T); (801 9417425 A G); (802 9417432 G A); (803 9417634 T C); (804
9417861 A C); (805 9418114 C T); (806 9418437 T C); (807 9418652 T C); (808 9419393 C T); (809
9419617 C T); (810 9419754 A G); (811 9419973 C T); (812 9420410 T G); (813 9420628 C T); (814
9420661 CA C); (815 9420729 A G); (816 9420977 T A); (817 9421184 A G); (818 9421479 T C); (819
9421616 T C); (820 9421848 A G); (821 9421874 G A); (822 9421885 A C); (823 9421914 C T); (824
9421988 C T); (825 9422055 C A); (826 9422117 G A); (827 9422178 T C); (828 9422281 G A); (829
9422315 C T); (830 9422905 T C); (831 9422971 A G); (832 9423460 G A); (833 9423546 T C); (834
9423550 G A); (835 9423581 C G); (836 9423680 T G); (837 9424297 C G); (838 9424406 G A); (839
9424450 G A); (840 9424653 G A); (841 9424696 T G); (842 9424747 C T); (843 9424926 A T); (844
9425106 A G); (845 9425320 C T); (846 9425363 T C); (847 9425521 A G); (848 9425554 T C); (849
9425628 T C); (850 9425670 T C); (851 9425681 G A); (852 9425717 C T); (853 9425992 A G); (854
9426759 T C); (855 9427769 C T); (856 9427795 A G); (857 9427891 C T); (858 9427918 G A); (859
9427922 G A); (860 9427964 A G); (861 9428165 C A); (862 9428353 A C); (863 9428431 A G); (864
9428435 G A); (865 9428486 G T); (866 9428550 T C); (867 9428895 G A); (868 9428899 A G); (869
9429022 G A); (870 9429038 A G); (871 9429057 T C); (872 9429347 T A); (873 9429404 T G); (874
9429421 A G); (875 9429428 A G); (876 9429449 G A); (877 9429524 G A); (878 9429532 T C); (879
9429684 A G); (880 9429801 A AT); (881 9429883 T G); (882 9430012 G A); (883 9430118 C T); (884
9430119 A G); (885 9430271 G C); (886 9430325 T C); (887 9430468 C T); (888 9430514 C T); (889
9430589 C T); (890 9430776 C CT); (891 9430982 C G); (892 9431302 C T); (893 9431576 T C); (894
9432407 TA T); (895 9432437 C G); (896 9432583 C G); (897 9432691 G A); (898 9432824 A AT); (899
9433535 C T); (900 9433664 G C); (901 9433809 T G); (902 9434341 A T); (903 9434558 T C); (904
9434653 C T); (905 9435162 C A); (906 9435398 A C); (907 9435450 T C); (908 9435461 T C); (909
9435753 A C); (910 9435767 T C); (911 9435821 C A); (912 9436059 T C); (913 9436160 A G); (914
9436307 T C); (915 9436558 T C); (916 9436704 T C); (917 9436802 C A); (918 9436930 C T); (919
9437053 C T); (920 9437153 G A); (921 9437246 G T); (922 9437284 T C); (923 9437421 C T); (924
9437441 G A); (925 9437442 C T); (926 9437510 G A); (927 9437525 A G); (928 9437557 C T); (929
9437697 A G); (930 9437852 C A); (931 9437870 C G); (932 9438007 TTA T); (933 9438099 TAGTTA
T); (934 9438352 T C); (935 9438524 T C); (936 9438544 T C); (937 9439324 C G); (938 9439365 C T); (939
9439462 A G); (940 9439484 C G); (941 9440833 C G); (942 9440984 T C); (943 9440986 A G); (944
9441004 T C); (945 9441028 G C); (946 9441529 C CTT); (947 9441763 C T); (948 9441939 A T); (949
9442039 C T); (950 9442502 C T); (951 9442508 C T); (952 9442860 A T); (953 9442951 A G); (954
9443455 C A); (955 9443658 C T); (956 9444001 A G); (957 9444274 T C); (958 9445091 T G); (959
9445345 T C); (960 9445355 T C); (961 9445487 G A); (962 9445549 A G); (963 9446029 C A); (964
9446263 A C); (965 9446475 A T); (966 9446853 C T); (967 9446962 A G); (968 9447166 G C); (969
9447489 C T); (970 9447490 G T); (971 9447698 T C); (972 9447780 G A); (973 9448177 G A); (974
9448860 A T); (975 9449685 G T); (976 9449708 T G); (977 9449816 A T); (978 9450229 C T); (979
9450491 A T); (980 9451200 A G); (981 9451514 C T); (982 9451778 A C); (983 9452256 T TA); (984
9452438 C T); (985 9452656 A AT); (986 9452719 T A); (987 9452807 T C); (988 9453097 A C); (989
9453247 C A); (990 9453275 G A); (991 9453294 T TA); (992 9453483 A G); (993 9453515 G A); (994
9453625 T C); (995 9453709 A G); (996 9453717 A G); (997 9453978 T C); (998 9453995 G A); (999
9454102 T C); (1000 9454213 T C); (1001 9454229 C T); (1002 9454260 GA G); (1003 9454295 G A); (1004
9454312 T C); (1005 9454326 GAATA G); (1006 9454404 G A); (1007 9454480 C T); (1008 9454593 C
T); (1009 9454725 C T); (1010 9454843 C T); (1011 9454881 T G); (1012 9454900 A ACT); (1013 9454957
A G); (1014 9454969 C T); (1015 9455067 T C); (1016 9455224 A AT); (1017 9455396 G T); (1018 9455408
C T); (1019 9455433 A C); (1020 9455631 G A); (1021 9455681 C T); (1022 9455688 A G); (1023 9455691
G A); (1024 9455768 A C); (1025 9455888 C T); (1026 9456022 A T); (1027 9456039 G A); (1028 9456118
A T); (1029 9456217 G A); (1030 9456355 T G); (1031 9456440 T C); (1032 9456494 C A); (1033 9456649
T G); (1034 9456784 C T); (1035 9456984 G A); (1036 9456999 A T); (1037 9457153 G A); (1038 9457204
G T); (1039 9457300 G A); (1040 9457437 C T); (1041 9457500 G A); (1042 9457544 C A); (1043 9457670
A C); (1044 9457694 A C); (1045 9457768 A T); (1046 9457852 G A); (1047 9457930 T G); (1048 9457941
T C); (1049 9458010 C T); (1050 9458026 T C); (1051 9458076 C T); (1052 9458100 C CT); (1053 9458177
G GGT); (1054 9458216 T C); (1055 9458217 A G); (1056 9458322 C T); (1057 9458440 G A); (1058
9458750 A G); (1059 9458792 G A); (1060 9458826 T C); (1061 9458827 C T); (1062 9458864 G A); (1063
9458868 C T); (1064 9458872 T C); (1065 9458895 C T); (1066 9458897 T C); (1067 9458908 C T); (1068
9458926 T G); (1069 9458953 T A); (1070 9459175 T C); (1071 9459191 C T); (1072 9460328 C G); (1073
9460416 A T); (1074 9460654 A G); (1075 9460785 A G); (1076 9460840 G
GCTGGGAATACTTTTTTT); (1077 9461200 A T); (1078 9461359 G A); (1079 9461476 C T); (1080 9461662
T A); (1081 9462635 T G); (1082 9462938 T C); (1083 9463111 T C); (1084 9463724 G A); (1085 9463725 T
A); (1086 9463789 T TGCA); (1087 9463798 G T); (1088 9463840 A C); (1089 9464187 CA C); (1090
9464908 C T); (1091 9465067 G GT); (1092 9465073 A G); (1093 9465130 G GA); (1094 9465475 G
C); (1095 9465923 C T); (1096 9466002 A G); (1097 9466025 C T); (1098 9466156 A C); (1099 9466214 A
G); (1100 9466677 G T); (1101 9466738 G C); (1102 9467246 G A); (1103 9467267 A T); (1104 9467469
TA T); (1105 9467791 A C); (1106 9468047 T A); (1107 9468288 G A); (1108 9468290 G A); (1109 9468292
G A); (1110 9468294 G A); (1111 9468296 G A); (1112 9468298 G A); (1113 9468300 A G); (1114 9468326
A G); (1115 9468332 A G); (1116 9468472 C T); (1117 9468538 G A); (1118 9468690 A G); (1119 9468751
C G); (1120 9468766 A G); (1121 9468776 C T); (1122 9469027 T A); (1123 9469599 C A); (1124 9469788
C A); (1125 9470182 G A); (1126 9470516 T C); (1127 9470605 T G); (1128 9470702 C T); (1129 9470724
A G); (1130 9470740 T C); (1131 9470754 A G); (1132 9470763 C T); (1133 9470794 G C); (1134 9471060
T C); (1135 9471073 G C); (1136 9471199 C G); (1137 9471261 A G); (1138 9471377 C G); (1139 9471460
T G); (1140 9471556 G T); (1141 9471911 C G); (1142 9471990 C G); (1143 9472128 A C); (1144 9472589
A C); (1145 9473201 T G); (1146 9473264 G C); (1147 9473307 C T); (1148 9473336 A G); (1149 9473370
A G); (1150 9473409 T C); (1151 9473432 T A); (1152 9473450 G A); (1153 9473600 A G); (1154 9473667
A G); (1155 9473775 C CAT); (1156 9473847 T C); (1157 9474043 C T); (1158 9474105 A T); (1159
9474264 A C); (1160 9474440 G A); (1161 9474536 C A); (1162 9474578 A G); (1163 9474878 G C); (1164
9474985 T C); (1165 9475083 A G); (1166 9475116 C T); (1167 9475674 G T); (1168 9475798 A AT); (1169
9475885 A G); (1170 9476065 G A); (1171 9476151 G A); (1172 9476444 G A); (1173 9476522 C T); (1174
9476639 T C); (1175 9476886 G A); (1176 9476893 T C); (1177 9476963 C T); (1178 9477136 A G); (1179
9477262 G A); (1180 9477462 G A); (1181 9477775 A C); (1182 9477821 C T); (1183 9477936 C T); (1184
9478024 T G); (1185 9478191 C T); (1186 9478379 G A); (1187 9478393 A G); (1188 9478465 G T); (1189
9478480 G A); (1190 9478599 A G); (1191 9478776 T C); (1192 9479265 C T); (1193 9479598 A G); (1194
9479765 G A); (1195 9480013 C A); (1196 9480020 T C); (1197 9480027 C T); (1198 9480171 C T); (1199
9480229 G A); (1200 9480394 G A); (1201 9480647 A G); (1202 9480998 C G); (1203 9481189 C T); (1204
9481238 A C); (1205 9481265 G A); (1206 9481433 G A); (1207 9481669 C T); (1208 9481717 C T); (1209
9481748 A G); (1210 9481789 C G); (1211 9481809 C T); (1212 9482008 G A); (1213 9482104 T C); (1214
9482170 T C); (1215 9482174 C T); (1216 9482276 T C); (1217 9482425 C T); (1218 9482465 G A); (1219
9482792 C T); (1220 9482816 C T); (1221 9482817 T C); (1222 9482830 A G); (1223 9483010 A G); (1224
9483063 T C); (1225 9483106 C T); (1226 9483109 C G); (1227 9483127 T C); (1228 9483206 T C); (1229
9483237 A G); (1230 9483298 G A); (1231 9483431 T C); (1232 9483497 C T); (1233 9483574 T C); (1234
9483758 C T); (1235 9483831 C T); (1236 9483837 C T); (1237 9483915 C T); (1238 9484026 T G); (1239
9484307 A C); (1240 9484360 C T); (1241 9484376 T C); (1242 9484496 G A); (1243 9484498 C T); (1244
9484568 C A); (1245 9484610 G T); (1246 9484738 G A); (1247 9484752 G A); (1248 9484768 T G); (1249
9484904 C T); (1250 9484936 A G); (1251 9484954 C T); (1252 9484991 T C); (1253 9485086 C T); (1254
9485422 CTT C); (1255 9485469 G A); (1256 9485523 C A); (1257 9485539 A G); (1258 9485579 T
C); (1259 9485689 C T); (1260 9485853 T A); (1261 9486124 G A); (1262 9486180 A G); (1263 9486391 A
G); (1264 9486394 A G); (1265 9486513 C T); (1266 9486515 C T); (1267 9486586 A T); (1268 9486593 T
A); (1269 9486648 C CAT); (1270 9486700 T C); (1271 9486735 T TGGATAACTAGAA); (1272 9486785 T
C); (1273 9487198 T C); (1274 9487413 C T); (1275 9487474 A T); (1276 9487566 T A); (1277 9487629 G
A); (1278 9487673 T C); (1279 9487683 A G); (1280 9487759 G A); (1281 9487884 G A); (1282 9488114 T
A); (1283 9488203 C T); (1284 9488210 C G); (1285 9488232 C T); (1286 9488248 G A); (1287 9488283 G
A); (1288 9488452 G A); (1289 9488495 T C); (1290 9488587 T C); (1291 9488616 G A); (1292 9488678 C
T); (1293 9488873 C G); (1294 9488893 A G); (1295 9489041 A G); (1296 9489077 A T); (1297 9489084 G
A); (1298 9489144 C G); (1299 9489155 C T); (1300 9489174 C T); (1301 9489275 A G); (1302 9489387 G
C); (1303 9489515 A G); (1304 9489535 A G); (1305 9489759 A G); (1306 9490131 G
GCGATCAGCGCGGGAACGGCA); (1307 9490154 G A); (1308 9490179 C T); (1309 9490220 A G); (1310
9490687 T C); (1311 9491064 C T); (1312 9491264 T C); (1313 9491304 C T); (1314 9491446 C T); (1315
9491495 T C); (1316 9491887 G A); (1317 9492012 A G); (1318 9492034 C T); (1319 9492089 A C); (1320
9492103 G A); (1321 9492316 T C); (1322 9492403 G A); (1323 9492559 A G); (1324 9492560 G A); (1325
9492610 G A); (1326 9492727 A G); (1327 9492949 GT G); (1328 9492962 G A); (1329 9493014 A
T); (1330 9493447 C T); (1331 9493477 G C); (1332 9493660 T C); (1333 9493899 G C); (1334 9493953 C
T); (1335 9493970 T C); (1336 9496792 A G); (1337 9497459 C T); (1338 9497670 T C); (1339 9497771 C
T); (1340 9497892 T A); (1341 9497920 G A); (1342 9497952 T C); (1343 9498036 C T); (1344 9498193 A
G); (1345 9498199 G A); (1346 9498291 C T); (1347 9498377 A C); (1348 9498551 A T); (1349 9498613 C
A); (1350 9498665 A C); (1351 9498693 A AAAATTC); (1352 9498699 T G); (1353 9498700 T A); (1354
9498762 G A); (1355 9498766 G A); (1356 9498769 C T); (1357 9498779 T C); (1358 9498854 A T); (1359
9498878 T C); (1360 9498927 A G); (1361 9499155 C T); (1362 9499438 G C); (1363 9499780 A AG); (1364
9500102 C A); (1365 9500312 T C); (1366 9500481 T C); (1367 9500849 A G); (1368 9500877 C G); (1369
9501404 A T); (1370 9501828 C T); (1371 9502018 T C); (1372 9502027 A G); (1373 9502088 T C); (1374
9502407 T TA); (1375 9502837 A C); (1376 9502845 A G); (1377 9503198 A T); (1378 9503199 A G); (1379
9503453 T C); (1380 9503766 A G); (1381 9503776 A G); (1382 9503842 T C); (1383 9503850 T C); (1384
9503917 A G); (1385 9504014 G A); (1386 9504037 G A); (1387 9504099 A G); (1388 9504104 G T); (1389
9504182 T C); (1390 9504194 C T); (1391 9504221 T C); (1392 9504299 C T); (1393 9504304 A G); (1394
9504316 T TG); (1395 9504375 A T); (1396 9504424 C T); (1397 9504436 A G); (1398 9504444 C T); (1399
9504498 A G); (1400 9504499 C T); (1401 9504524 ACTT A); (1402 9504586 A G); (1403 9504695 T
C); (1404 9504807 C A); (1405 9505048 A G); (1406 9505115 G GAA); (1407 9505179 T C); (1408 9505206
T C); (1409 9505209 T C); (1410 9505296 A G); (1411 9505366 C T); (1412 9505413 A G); (1413 9505560
C T); (1414 9505705 T C); (1415 9505789 C T); (1416 9505820 A T); (1417 9505897 A G); (1418 9506143 C
T); (1419 9506153 C T); (1420 9506539 C T); (1421 9506757 A T); (1422 9506789 A G); (1423 9506878 T
C); (1424 9506947 T C); (1425 9506983 G A); (1426 9507102 A G); (1427 9507135 C T); (1428 9507228 C
A); (1429 9507684 C T); (1430 9507985 A G); (1431 9507996 A C); (1432 9508018 C G); (1433 9508045 C
T); (1434 9508063 G T); (1435 9508214 T C); (1436 9508355 A G); (1437 9508413 G A); (1438 9508647 G
A); (1439 9508675 C A); (1440 9508676 C A); (1441 9508703 T C); (1442 9508748 T C); (1443 9508761 A
G); (1444 9508814 G A); (1445 9508950 A AT); (1446 9508956 G A); (1447 9508990 G A); (1448 9509040
C G); (1449 9509041 T C); (1450 9509251 G A); (1451 9509257 G C); (1452 9509573 A T); (1453 9509656
G C); (1454 9509694 T C); (1455 9509891 G A); (1456 9509974 A G); (1457 9510072 T C); (1458 9510170
A C); (1459 9510228 T C); (1460 9510259 T C); (1461 9510322 G A); (1462 9510359 C T); (1463 9510410 A
T); (1464 9510451 A G); (1465 9510460 C T); (1466 9510521 T G); (1467 9510681 G A); (1468 9510878 A
G); (1469 9511307 A G); (1470 9511388 C T); (1471 9511450 G A); (1472 9511499 C T); (1473 9511544 C
A); (1474 9511699 G A); (1475 9511710 A G); (1476 9511735 T C); (1477 9511757 T C); (1478 9511819 G
GCGGTA); (1479 9511862 A C); (1480 9511931 G T); (1481 9511935 C T); (1482 9512066 A G); (1483
9512088 T C); (1484 9512155 A G); (1485 9512190 T C); (1486 9512201 C T); (1487 9512346 G A); (1488
9512394 C T); (1489 9512959 T A); (1490 9513054 A G); (1491 9513288 TAAG T); (1492 9514011 T
C); (1493 9514033 C CT); (1494 9514398 A G); (1495 9514458 CTT C); (1496 9514698 A AT); (1497
9514729 T G); (1498 9515754 T C); (1499 9515838 C T); (1500 9515964 G C); (1501 9516118 A C); (1502
9516287 A G); (1503 9516691 G T); (1504 9517218 G C); (1505 9518352 T C); (1506 9518782 A C); (1507
9518814 G T); (1508 9518901 G A); (1509 9518911 G A); (1510 9519142 C T); (1511 9519974 T G); (1512
9520459 C CTCATG); (1513 9520537 C T); (1514 9520548 C T); (1515 9520622 G A); (1516 9520716 T
C); (1517 9520831 C T); (1518 9520998 G A); (1519 9523230 A C); (1520 9525277 T A); (1521 9525623 C
T); (1522 9526027 C G); (1523 9526860 T C); (1524 9526915 A G); (1525 9526978 T C); (1526 9526996 A
G); (1527 9526998 G T); (1528 9527386 A C); (1529 9527707 T C); (1530 9527945 T C); (1531 9528505 G
A); (1532 9528549 C T); (1533 9528569 C T); (1534 9528977 T C); (1535 9529022 C T); (1536 9529190 G
A); (1537 9529288 C T); (1538 9531362 G A); (1539 9531521 C G); (1540 9531589 A G); (1541 9532520 C
CATAT); (1542 9533329 A T); (1543 9533441 C A); (1544 9533767 T A); (1545 9533797 T TTA); (1546
9534097 A T); (1547 9534241 C G); (1548 9534463 TG T); (1549 9535274 G GA); (1550 9535309 G
A); (1551 9535436 C G); (1552 9535582 C A); (1553 9535652 C T); (1554 9535660 G A); (1555 9535719 C
T); (1556 9536221 C T); (1557 9536360 C T); (1558 9536367 A G); (1559 9536511 C T); (1560 9536597 TA
T); (1561 9536648 A T); (1562 9536863 G A); (1563 9537084 A G); (1564 9537281 A T); (1565 9537334 T
C); (1566 9537345 G A); (1567 9537442 T G); (1568 9538176 A T); (1569 9538292 C T); (1570 9538639 G
A); (1571 9538645 C T); (1572 9538715 C T); (1573 9538718 G A); (1574 9538744 G A); (1575 9538774 C
A); (1576 9538923 A G); (1577 9538950 T C); (1578 9538968 G A); (1579 9538980 C CTGTTAT); (1580
9538991 C T); (1581 9538992 C T); (1582 9539051 C T); (1583 9539063 G A); (1584 9539074 C T); (1585
9539097 T A); (1586 9539114 C G); (1587 9539115 T C); (1588 9539817 G A); (1589 9544888
TCTTCTCCGGCGC T); (1590 9544891 T C); (1591 9544895 T C); (1592 9544929 C T); (1593 9544970 C
G); (1594 9545002 G A); (1595 9545005 C T); (1596 9545897 T G); (1597 9546595 AT A); (1598 9547411
G T); (1599 9547823 A G); (1600 9548626 TA T); (1601 9548753 T A); (1602 9548754 T A); (1603 9549747
C CT); (1604 9550135 T A); (1605 9550136 T G); (1606 9550137 A T); (1607 9550138 T A); (1608 9550139
T A); (1609 9550140 C T); (1610 9550141 A T); (1611 9550142 A T); (1612 9550143 T C); (1613 9550144 A
T); (1614 9550145 A T); (1615 9550146 G T); (1616 9550148 A T); (1617 9550149 A T); (1618 9550150 T
C); (1619 9550151 T A); (1620 9550152 G A); (1621 9550153 A C); (1622 9550154 T A); (1623 9550155 T
A); (1624 9550156 A G); (1625 9550298 G GT); (1626 9551455 G C); (1627 9551798 C T); (1628 9552614 T
A); (1629 9554823 T A); (1630 9555417 C T); (1631 9556253 G A); (1632 9556385 A T); (1633 9556386 C
A); (1634 9556387 A T); (1635 9558434 G A); (1636 9558518 C T); (1637 9559744 G T); (1638 9559773 G
T); (1639 9559782 C T); (1640 9560469 C T); (1641 9560874 C T); (1642 9562241 G T); (1643 9562375 G
A); (1644 9562572 AG A); (1645 9562870 G A); (1646 9562939 G T); (1647 9563011 C T); (1648 9563060
C A); (1649 9563143 G A); (1650 9564449 G A); (1651 9565117 G A); (1652 9565607 G A); (1653 9565743
C T); (1654 9566178 G T); (1655 9566472 G A); (1656 9566473 G A); (1657 9566992 C T); (1658 9569283
C T); (1659 9569317 C A); (1660 9570557 A T); (1661 9570604 A G); (1662 9571083 C T); (1663 9571204 C
T); (1664 9571225 G C); (1665 9571233 G C); (1666 9571315 C T); (1667 9571373 T C); (1668 9571391 A
G); (1669 9572717 T G); (1670 9572749 C T); (1671 9572886 G T); (1672 9572908 TTTCTTCCTAC T); (1673
9573307 T C); (1674 9573312 C T); (1675 9573504 C T); (1676 9575943 A T); (1677 9576136 G A); (1678
9576153 A G); (1679 9576183 A T); (1680 9576192 C T); (1681 9576279 C T); (1682 9576355 A T); (1683
9576437 A T); (1684 9576442 T C); (1685 9576479 C T); (1686 9576758 G A); (1687 9576894 C T); (1688
9577147 G A); (1689 9577219 A T); (1690 9577610 G A); (1691 9578749 C T); (1692 9578841 G A); (1693
9578892 C T); (1694 9578908 T G); (1695 9578966 G A); (1696 9580507 G A); (1697 9583392 T A); (1698
9584039 TAA T); (1699 9584080 T A); (1700 9584596 G A); (1701 9584994 G T); (1702 9585677 T
TTA); (1703 9585956 C T); (1704 9586568 G A); (1705 9586979 G A); (1706 9587180 A T); (1707 9587369
G A); (1708 9587448 G A); (1709 9587594 G A); (1710 9587872 G A); (1711 9587881 C T); (1712 9588310
T G); (1713 9588543 T C); (1714 9588861 A C); (1715 9589152 G GA); (1716 9590402 A C); (1717 9594279
C G); (1718 9595300 A G); (1719 9595455 G A); (1720 9596138 T C); (1721 9596514 G A); (1722 9596676
G A); (1723 9597148 T G); (1724 9605988 G C); (1725 9607748 C T); (1726 9607813 A C); (1727 9608272
T A); (1728 9608382 A C); (1729 9608414 T C); (1730 9608525 C T); (1731 9608997 C G); (1732 9609015 C
T); (1733 9609276 C A); (1734 9609318 C G); (1735 9609572 C T); (1736 9610804 G A); (1737 9611360 C
A); (1738 9611414 G T); (1739 9611483 G A); (1740 9612311 C T); (1741 9612505 G A); (1742 9612530 C
A); (1743 9612635 C T); (1744 9612678 G A); (1745 9612707 G A); (1746 9613211 C A); (1747 9613303 C
C T); (1748 9613905 G A); (1749 9614094 A AT); (1750 9614815 A C); (1751 9615257 T C); (1752 9615292
A T); (1753 9616858 A AT); (1754 9617712 C A); (1755 9617836 G A); (1756 9618036 T A); (1757 9618346
T C); (1758 9618625 A C); (1759 9618803 A T); (1760 9619001 A C); (1761 9620803 A C); (1762 9621209
G A); (1763 9621811 G GA); (1764 9621982 A G); (1765 9622088 A G); (1766 9622760 T G); (1767
9622986 G T); (1768 9623977 G A); (1769 9623997 C T); (1770 9624189 C T); (1771 9624824 G GA); (1772
9625058 C T); (1773 9625308 G A); (1774 9626222 T C); (1775 9626537 C A); (1776 9626627 G A); (1777
9626660 G A); (1778 9626876 A G); (1779 9627111 A G); (1780 9627250 A G); (1781 9627296 A G); (1782
9627665 C T); (1783 9627697 A G); (1784 9627976 G A); (1785 9628295 C G); (1786 9628491 C T); (1787
9628493 C T); (1788 9628498 G A); (1789 9628640 A C); (1790 9628732 T C); (1791 9628810 T C); (1792
9628819 C T); (1793 9628865 A G); (1794 9628909 C CT); (1795 9628934 A G); (1796 9628959 A C); (1797
9629010 C T); (1798 9629269 G A); (1799 9629376 G A); (1800 9629413 T C); (1801 9629459 C A); (1802
9629575 G A); (1803 9629700 C T); (1804 9629704 C A); (1805 9629739 C T); (1806 9629748 C T); (1807
9629775 A G); (1808 9629875 A G); (1809 9630202 T C); (1810 9630279 A G); (1811 9630353 A G); (1812
9630783 C T); (1813 9630882 T G); (1814 9630907 T C); (1815 9631000 C T); (1816 9631068 G A); (1817
9631078 A G); (1818 9631091 C T); (1819 9631305 A G); (1820 9631397 A G); (1821 9631496 G A); (1822
9631786 C T); (1823 9631808 C T); (1824 9631836 G A); (1825 9631952 A G); (1826 9632140 T C); (1827
9632186 G A); (1828 9632720 A G); (1829 9632866 C T); (1830 9632882 C T); (1831 9632976 T C); (1832
9633027 G T); (1833 9633345 T C); (1834 9633400 T C); (1835 9633409 C T); (1836 9633413 G A); (1837
9634720 T C); (1838 9634806 G C); (1839 9634825 A G); (1840 9634983 C T); (1841 9635321 G A); (1842
9635376 A G); (1843 9635427 A G); (1844 9635577 T C); (1845 9635632 T C); (1846 9635658 G C); (1847
9635819 A G); (1848 9635966 C T); (1849 9636210 T C); (1850 9636314 A G); (1851 9636357 G A); (1852
9636428 A G); (1853 9636457 T C); (1854 9636547 C T); (1855 9636566 C T); (1856 9636976 A G); (1857
9637145 T C); (1858 9637171 A C); (1859 9637522 T C); (1860 9637591 A G); (1861 9637628 G A); (1862
9637674 C T); (1863 9638002 C T); (1864 9638018 C T); (1865 9638086 G A); (1866 9638092 A C); (1867
9638169 C T); (1868 9638171 G C); (1869 9638242 T C); (1870 9638462 C T); (1871 9638526 C T); (1872
9638637 C T); (1873 9638710 G A); (1874 9638774 T C); (1875 9639378 C T); (1876 9639525 T G); (1877
9640041 G T); (1878 9640834 G A); (1879 9641144 A G); (1880 9641357 T TTTA); (1881 9642630 G
T); (1882 9643058 G A); (1883 9643942 A G); (1884 9643945 G A); (1885 9643993 A G); (1886 9644031 C
T); (1887 9644046 C T); (1888 9644651 T C); (1889 9644725 A T); (1890 9645020 A G); (1891 9645278 T
TA); (1892 9645337 T C); (1893 9645440 G A); (1894 9645471 C T); (1895 9645632 C T); (1896 9645731 G
A); (1897 9646011 C A); (1898 9646102 G C); (1899 9646346 A G); (1900 9646612 G C); (1901 9647217 T
C); (1902 9647412 G A); (1903 9647617 T A); (1904 9647943 A G); (1905 9648336 T A); (1906 9648754 G
A); (1907 9649050 T A); (1908 9649234 T C); (1909 9649501 G A); (1910 9649570 T C); (1911 9649641 C
T); (1912 9649750 T G); (1913 9649928 A C); (1914 9650449 A T); (1915 9650492 A G); (1916 9651128 T
C); (1917 9651139 C T); (1918 9651202 G C); (1919 9651530 C G); (1920 9651658 A C); (1921 9651804 C
T); (1922 9651899 T C); (1923 9652008 T C); (1924 9652156 G A); (1925 9652280 A G); (1926 9652465 A
G); (1927 9652575 T C); (1928 9652805 A G); (1929 9652865 A C); (1930 9652915 G A); (1931 9652951 A
G); (1932 9652957 C T); (1933 9653130 T C); (1934 9653152 G A); (1935 9654318 C T); (1936 9654524 A
G); (1937 9654552 G A); (1938 9654570 A G); (1939 9654576 C A); (1940 9654687 G A); (1941 9655793 C
T); (1942 9655912 G A); (1943 9655943 C T); (1944 9656007 G A); (1945 9656219 T G); (1946 9656338 A
G); (1947 9656363 A C); (1948 9656511 C T); (1949 9656730 G A); (1950 9656771 CTG C); (1951 9656796
C T); (1952 9656808 A G); (1953 9656826 T C); (1954 9656948 G A); (1955 9657055 T C); (1956 9657227
A C); (1957 9657451 G A); (1958 9657461 T C); (1959 9657916 A G); (1960 9658346 C T); (1961 9658690
T C); (1962 9658695 G A); (1963 9658723 C T); (1964 9658776 A G); (1965 9658952 A G); (1966 9659543
A C); (1967 9660024 G A); (1968 9661058 G A); (1969 9661093 C CACTTCCCTCCTTTAAA); (1970 9661176
C A); (1971 9661205 A C); (1972 9661419 G A); (1973 9661567 T G); (1974 9661592 G A); (1975 9661638
G C); (1976 9662044 GA G); (1977 9662363 T A); (1978 9662528 C A); (1979 9662716 AT A); (1980
9663388 G A); (1981 9663392 T C); (1982 9663603 G A); (1983 9663689 A G); (1984 9663725 C T); (1985
9663791 C T); (1986 9663805 A T); (1987 9663827 G A); (1988 9663997 A C); (1989 9664272 A G); (1990
9664274 T C); (1991 9664369 G A); (1992 9664479 T C); (1993 9664624 G A); (1994 9664753 G A); (1995
9664928 A G); (1996 9664942 A G); (1997 9664993 T C); (1998 9665042 G A); (1999 9665066 A G); (2000
9665067 T A); (2001 9665068 A G); (2002 9665149 C T); (2003 9665175 T C); (2004 9665325 A C); (2005
9665371 A G); (2006 9665451 G T); (2007 9665464 G A); (2008 9665521 C T); (2009 9665534 C T); (2010
9665564 A G); (2011 9665721 G A); (2012 9665737 A G); (2013 9665773 T G); (2014 9665779 G A); (2015
9665790 C T); (2016 9665870 C T); (2017 9665879 A G); (2018 9665971 A T); (2019 9666010 C A); (2020
9666014 T G); (2021 9666017 G A); (2022 9666175 A G); (2023 9666189 T C); (2024 9666253 A G); (2025
9666261 A C); (2026 9666462 G A); (2027 9666570 C A); (2028 9666676 A G); (2029 9666685 G T); (2030
9666687 A G); (2031 9666718 A G); (2032 9666894 A G); (2033 9667039 G A); (2034 9667041 G T); (2035
9668015 C G); (2036 9668224 G T); (2037 9668259 A G); (2038 9668290 G A); (2039 9668292 G A); (2040
9668478 T C); (2041 9668498 G T); (2042 9668613 G GA); (2043 9668782 T C); (2044 9668833 G A); (2045
9668958 C A); (2046 9668961 A G); (2047 9669128 G A); (2048 9669150 T C); (2049 9669413 C T); (2050
9669454 T C); (2051 9669494 C T); (2052 9669512 C G); (2053 9669532 T C); (2054 9669740 T A); (2055
9669814 T C); (2056 9669815 G T); (2057 9670067 G A); (2058 9670082 G A); (2059 9670088 C T); (2060
9670135 A G); (2061 9670153 G A); (2062 9670162 G A); (2063 9670232 A G); (2064 9670240 C T); (2065
9670257 G A); (2066 9670259 T C); (2067 9670393 C G); (2068 9670413 A G); (2069 9670817 C T); (2070
9670846 G A); (2071 9670888 A C); (2072 9670982 G T); (2073 9671044 C T); (2074 9671110 T C); (2075
9671206 T C); (2076 9671318 A T); (2077 9671360 G A); (2078 9671477 G A); (2079 9671511 G A); (2080
9671593 G C); (2081 9671835 A G); (2082 9671841 GA G); (2083 9671901 G A); (2084 9671924 A
C); (2085 9671958 T A); (2086 9672128 C G); (2087 9672161 G A); (2088 9672420 A G); (2089 9672519 T
C); (2090 9672527 C T); (2091 9672845 T C); (2092 9672856 G A); (2093 9673057 C T); (2094 9673093 C
T); (2095 9673187 A C); (2096 9673275 A G); (2097 9673574 G A); (2098 9673771 A G); (2099 9673877 A
G); (2100 9673983 C G); (2101 9674282 T C); (2102 9674290 T C); (2103 9674371 A G); (2104 9674663 T
C); (2105 9674676 T C); (2106 9674688 T C); (2107 9674710 A C); (2108 9675415 G T); (2109 9675490 G
A); (2110 9675744 A G); (2111 9675967 T C); (2112 9676012 C G); (2113 9676043 A T); (2114 9676084 G
C); (2115 9676091 T C); (2116 9676152 A T); (2117 9676257 T G); (2118 9676459 T TTC); (2119 9676598 T
A); (2120 9676857 T G); (2121 9677150 C T); (2122 9677171 T C); (2123 9677213 T C); (2124 9677288 T
C); (2125 9677402 A G); (2126 9677609 C A); (2127 9677862 C T); (2128 9678021 C T); (2129 9678283 T
C); (2130 9678536 T A); (2131 9678799 A T); (2132 9679098 G A); (2133 9679182 G A); (2134 9679223 A
G); (2135 9679273 G T); (2136 9679291 A C); (2137 9679389 G A); (2138 9679955 G A); (2139 9680000
CT C); (2140 9680224 A C); (2141 9680244 G A); (2142 9680837 A G); (2143 9680868 G T); (2144 9680869
G T); (2145 9680894 C T); (2146 9681251 C T); (2147 9681361 C T); (2148 9681678 C A); (2149 9681715 A
C); (2150 9681963 A C); (2151 9682235 G A); (2152 9682407 A G); (2153 9682433 T C); (2154 9682467 A
G); (2155 9682552 C T); (2156 9682821 T G); (2157 9682841 T A); (2158 9682870 T C); (2159 9682904 G
A); (2160 9682932 T C); (2161 9683157 A G); (2162 9683185 T C); (2163 9683447 A G); (2164 9683514 T
C); (2165 9683531 G A); (2166 9683588 A G); (2167 9683759 C A); (2168 9683867 T C); (2169 9683899 A
G); (2170 9684151 G A); (2171 9684248 C A); (2172 9684251 G T); (2173 9684440 C G); (2174 9684475 C
A); (2175 9684663 A C); (2176 9684934 G A); (2177 9685182 A C); (2178 9685417 T C); (2179 9685531 C
T); (2180 9685577 G A); (2181 9685890 A C); (2182 9685933 T C); (2183 9686034 C G); (2184 9686219 G
A); (2185 9686220 A G); (2186 9686342 G A); (2187 9686343 A G); (2188 9686712 C T); (2189 9686860 T
G); (2190 9686964 T C); (2191 9687026 T C); (2192 9687457 G A); (2193 9687937 C G); (2194 9687946 G
A); (2195 9687958 C T); (2196 9688071 C A); (2197 9688241 T C); (2198 9688244 T C); (2199 9688267 G
C); (2200 9688420 A G); (2201 9688958 A G); (2202 9689032 A G); (2203 9689086 G A); (2204 9689208 T
C); (2205 9689304 A T); (2206 9689472 G A); (2207 9689504 G C); (2208 9689663 C T); (2209 9689702 T
C); (2210 9689804 G A); (2211 9689931 G A); (2212 9690056 C T); (2213 9690193 G A); (2214 9690264 T
TA); (2215 9690265 G A); (2216 9690562 A C); (2217 9690586 A G); (2218 9690591 T C); (2219 9690794 C
T); (2220 9690934 T C); (2221 9690979 G A); (2222 9690987 G A); (2223 9690989 T C); (2224 9691103 G
A); (2225 9691394 A G); (2226 9691837 A G); (2227 9691882 T C); (2228 9691914 A G); (2229 9692003 G
A); (2230 9692035 A T); (2231 9692143 A G); (2232 9692416 C CG); (2233 9692442 T G); (2234 9692454
A G); (2235 9692739 G A); (2236 9692790 G A); (2237 9692810 G A); (2238 9693056 T C); (2239 9693337
G A); (2240 9693518 C T); (2241 9693545 G A); (2242 9693587 T G); (2243 9693598 T C); (2244 9694127
G C); (2245 9694166 C T); (2246 9694258 G A); (2247 9694515 G GTTATAATTATTTA); (2248 9694526 G
T); (2249 9694528 T C); (2250 9694682 G T); (2251 9694749 G A); (2252 9694969 C T); (2253 9695114 G
T); (2254 9695117 G A); (2255 9695162 G A); (2256 9695420 A T); (2257 9695444 G A); (2258 9695481 C
A); (2259 9695820 A G); (2260 9696130 G A); (2261 9696193 C A); (2262 9696239 T C); (2263 9696287 A
T); (2264 9696535 GCATAAATCAATGGTTTTTCAGTCAAA G); (2265 9696735 A G); (2266 9697249 A
C); (2267 9697500 C T); (2268 9697766 T A); (2269 9697975 C T); (2270 9697980 G C); (2271 9698012 G
A); (2272 9698030 C G); (2273 9698177 CA C); (2274 9698937 A C); (2275 9699128 G T); (2276 9699181
G A); (2277 9699463 C T); (2278 9699503 G A); (2279 9699538 G A); (2280 9699848 C T); (2281 9699891
G C); (2282 9700062 A T); (2283 9700209 G A); (2284 9700450 C T); (2285 9700464 T A); (2286 9700925
T C); (2287 9700979 T G); (2288 9701043 T A); (2289 9701406 G C); (2290 9701453 G C); (2291 9701797
T C); (2292 9702063 A G); (2293 9702105 A G); (2294 9702840 C T); (2295 9702865 A G); (2296 9703110
G GT); (2297 9703225 A G); (2298 9703424 C G); (2299 9703480 A AAATCAG); (2300 9703502 T A); (2301
9703556 G A); (2302 9703591 G C); (2303 9703622 A G); (2304 9703629 G T); (2305 9703634 G T); (2306
9704124 A T); (2307 9704202 T C); (2308 9704281 T C); (2309 9704342 T C); (2310 9704510 A G); (2311
9704619 G A); (2312 9704654 A G); (2313 9704760 T C); (2314 9704777 G C); (2315 9704780 C A); (2316
9704781 T A); (2317 9704782 A G); (2318 9705674 C T); (2319 9705733 A G); (2320 9706026 G
GATTGCATAATAGAACTTTAAAAAACATAAA); (2321 9706142 G T); (2322 9706178 T C); (2323 9706205 C
T); (2324 9706210 C G); (2325 9706540 T A); (2326 9706879 A G); (2327 9706939 T C); (2328 9707093 A
G); (2329 9707196 A G); (2330 9707214 C T); (2331 9707307 A G); (2332 9707537 A C); (2333 9707618 A
T); (2334 9707688 T C); (2335 9707789 A G); (2336 9707865 C T); (2337 9708027 G A); (2338 9709607 C
A); (2339 9710169 T C); (2340 9710294 T C); (2341 9710717 C A); (2342 9710725 T C); (2343 9710728 T
C); (2344 9710737 G A); (2345 9710766 C T); (2346 9710868 AG A); (2347 9710906 C A); (2348 9710914
G A); (2349 9710923 T C); (2350 9711037 A G); (2351 9711157 C A); (2352 9711428 A G); (2353 9711455
A G); (2354 9711957 A T); (2355 9711964 C T); (2356 9711982 A G); (2357 9712039 A G); (2358 9712070
C T); (2359 9713457 G A); (2360 9713772 A G); (2361 9713832 A G); (2362 9714117 T
TTCACAACATGAAAATC); (2363 9714225 A G); (2364 9714263 C T); (2365 9714278 A G); (2366 9714385 A
G); (2367 9714759 T C); (2368 9714817 AG A); (2369 9714886 G A); (2370 9714941 T C); (2371 9715318
G T); (2372 9715495 C T); (2373 9715504 G A); (2374 9715887 T G); (2375 9716140 T C); (2376 9716185
G T); (2377 9716302 G A); (2378 9716507 A G); (2379 9716629 A T); (2380 9716648 A C); (2381 9716804
A T); (2382 9716911 C A); (2383 9717220 G A); (2384 9717531 A G); (2385 9717985 A G); (2386 9718115
C T); (2387 9718341 C G); (2388 9718429 G C); (2389 9718749 G T); (2390 9718793 A AC); (2391 9719396
A G); (2392 9719434 A T); (2393 9719704 G A); (2394 9719823 A G); (2395 9719971 A G); (2396 9720350
C G); (2397 9720394 C A); (2398 9720399 G A); (2399 9720857 GA G); (2400 9720897 T C); (2401
9721013 C T); (2402 9721044 G A); (2403 9721102 A G); (2404 9721291 C T); (2405 9721330 C T); (2406
9721412 T C); (2407 9721548 C T); (2408 9721878 G A); (2409 9721941 A T); (2410 9722022 T A); (2411
9722116 G A); (2412 9722172 T C); (2413 9722212 A G); (2414 9722421 T C); (2415 9722612 C T); (2416
9722637 C T); (2417 9722829 T C); (2418 9722922 T C); (2419 9723286 T C); (2420 9723297 T C); (2421
9723595 A G); (2422 9723599 G A); (2423 9723620 T G); (2424 9723986 C T); (2425 9724085 C T); (2426
9724086 G C); (2427 9724142 AT A); (2428 9724371 T A); (2429 9724373 A T); (2430 9725330 G C); (2431
9725366 A G); (2432 9725799 G A); (2433 9725827 G A); (2434 9725893 T C); (2435 9726581 G A); (2436
9726723 G C); (2437 9726765 C T); (2438 9726799 T C); (2439 9726969 TA T); (2440 9727046 C T); (2441
9727103 A G); (2442 9727111 G A); (2443 9727119 G A); (2444 9727225 C T); (2445 9727232 A T); (2446
9727237 C CT); (2447 9727284 A G); (2448 9727324 G A); (2449 9727356 T C); (2450 9727401 G A); (2451
9727500 C T); (2452 9727644 T G); (2453 9727919 C T); (2454 9727991 C T); (2455 9728047 T G); (2456
9728253 A G); (2457 9728303 A G); (2458 9728333 G C); (2459 9728406 T C); (2460 9728445 A G); (2461
9728600 G A); (2462 9729120 T G); (2463 9729191 G C); (2464 9729397 A AT); (2465 9729465 A T); (2466
9729846 A C); (2467 9729911 G A); (2468 9730519 C T); (2469 9730558 T TA); (2470 9730584 G A); (2471
9730600 C T); (2472 9730622 G A); (2473 9730831 T C); (2474 9730916 C T); (2475 9731075 G A); (2476
9731123 A T); (2477 9731277 A C); (2478 9731291 A G); (2479 9731420 G A); (2480 9731471 TA T); (2481
9731888 AT A); (2482 9732299 C A); (2483 9732353 A G); (2484 9732354 C T); (2485 9732370 A G); (2486
9732441 C T); (2487 9732469 C A); (2488 9732662 A G); (2489 9732693 T C); (2490 9732758 A G); (2491
9732837 G A); (2492 9733040 C T); (2493 9733068 CA C); (2494 9733118 G A); (2495 9733786 T C); (2496
9733942 T C); (2497 9734246 AT A); (2498 9734317 G A); (2499 9734675 C T); (2500 9734740 C A); (2501
9734878 A T); (2502 9735040 A G); (2503 9735161 A T); (2504 9735371 A T); (2505 9735613 C CT); (2506
9735640 A C); (2507 9735666 A T); (2508 9735679 T C); (2509 9735747 A T); (2510 9735998 C T); (2511
9736327 T C); (2512 9736448 C A); (2513 9736512 C T); (2514 9736554 T C); (2515 9736605 C CA); (2516
9737293 T C); (2517 9737319 A C); (2518 9737469 T C); (2519 9737497 A G); (2520 9737507 T C); (2521
9737570 T A); (2522 9737692 A G); (2523 9737931 C T); (2524 9737938 C T); (2525 9737978 G A); (2526
9738041 G A); (2527 9738139 T A); (2528 9738347 T C); (2529 9738631 G A); (2530 9738646 A C); (2531
9738748 G A); (2532 9738931 A C); (2533 9739346 T G); (2534 9739563 T C); (2535 9739706 A G); (2536
9739709 C T); (2537 9739734 T A); (2538 9739805 A G); (2539 9739807 T G); (2540 9739942 T C); (2541
9739968 A G); (2542 9740019 G A); (2543 9740235 A G); (2544 9740340 G A); (2545 9740857 G C); (2546
9740919 C T); (2547 9740937 C T); (2548 9740980 G A); (2549 9741082 T G); (2550 9741194 A G); (2551
9741726 C T); (2552 9743316 G A); (2553 9744960 T C); (2554 9744987 A C); (2555 9745001 A G); (2556
9745022 A T); (2557 9745260 C A); (2558 9745396 G A); (2559 9745495 A G); (2560 9745589 G T); (2561
9745767 A G); (2562 9745852 C T); (2563 9745869 T C); (2564 9745926 C A); (2565 9745985 C T); (2566
9746281 T C); (2567 9746790 G A); (2568 9746825 G A); (2569 9746955 G A); (2570 9747149 C T); (2571
9747218 C CT); (2572 9747818 A C); (2573 9748048 A C); (2574 9748308 T C); (2575 9748591 T C); (2576
9748650 C T); (2577 9748769 T C); (2578 9748859 T C); (2579 9748905 C A); (2580 9749089 T C); (2581
9749178 G T); (2582 9749195 G A); (2583 9749229 C G); (2584 9749332 T C); (2585 9749460 T C); (2586
9749693 G T); (2587 9750209 C T); (2588 9766317 T C); (2589 9766662 T C); (2590 9766788 G A); (2591
9766859 C A); (2592 9767038 T C); (2593 9767100 G A); (2594 9767124 T C); (2595 9767285 A G); (2596
9767519 A G); (2597 9767753 C T); (2598 9767799 G A); (2599 9768102 T C); (2600 9768203 T G); (2601
9768426 A C); (2602 9768471 T C); (2603 9768828 G A); (2604 9768931 G A); (2605 9769267 G T); (2606
9769435 G A); (2607 9769727 T C); (2608 9769878 A AGTGGCTTGGGCATGGTTT); (2609 9770022 G
A); (2610 9770090 GA G); (2611 9770247 T C); (2612 9770430 G A); (2613 9770538 A C); (2614 9770562
A C); (2615 9770962 C G); (2616 9771115 T C); (2617 9771131 T G); (2618 9771236 T C); (2619 9771239 T
C); (2620 9771318 G C); (2621 9771887 T C); (2622 9771947 T A); (2623 9772063 G A); (2624 9772096 C
A); (2625 9772171 G A); (2626 9772237 T C); (2627 9772281 G A); (2628 9772282 A G); (2629 9772364 T
C); (2630 9772547 G A); (2631 9772560 T C); (2632 9772766 T C); (2633 9772786 A G); (2634 9772792 C
T); (2635 9772793 A G); (2636 9772794 C T); (2637 9772824 C T); (2638 9772842 T C); (2639 9772884 A
G); (2640 9772910 C T); (2641 9772958 C A); (2642 9772985 A G); (2643 9773000 A G); (2644 9773084 T
C); (2645 9773342 A G); (2646 9773977 A G); (2647 9774056 A G); (2648 9774096 A T); (2649 9774193 C
T); (2650 9774217 A G); (2651 9774458 C T); (2652 9774461 A G); (2653 9774650 C T); (2654 9774744 G
A); (2655 9774755 G T); (2656 9774757 C T); (2657 9774771 C T); (2658 9774982 A C); (2659 9775019 G
A); (2660 9775140 A G); (2661 9775145 A G); (2662 9775199 C T); (2663 9775339 G C); (2664 9775343 T
C); (2665 9775528 T C); (2666 9775586 G A); (2667 9775654 A T); (2668 9775747 T C); (2669 9775854 G
A); (2670 9775877 T C); (2671 9776066 G A); (2672 9776193 C T); (2673 9776270 T A); (2674 9776467 A
C); (2675 9776533 A G); (2676 9776549 C T); (2677 9776606 T C); (2678 9776608 G A); (2679 9776649 C
T); (2680 9776750 A C); (2681 9776864 C T); (2682 9777148 C T); (2683 9777157 G A); (2684 9777176 C
T); (2685 9777254 G A); (2686 9777313 G A); (2687 9777354 C CA); (2688 9777474 C A); (2689 9777503 T
C); (2690 9777684 T G); (2691 9777725 C T); (2692 9777767 C T); (2693 9777781 G C); (2694 9777900 T
A); (2695 9777901 A G); (2696 9778137 T C); (2697 9778198 A G); (2698 9778219 C T); (2699 9778514 C
T); (2700 9778585 A G); (2701 9778641 G A); (2702 9778708 T C); (2703 9778824 A G); (2704 9778965 C
T); (2705 9779007 G A); (2706 9779149 G A); (2707 9779365 C T); (2708 9779460 C A); (2709 9779492 G
T); (2710 9779508 A G); (2711 9779562 A G); (2712 9780120 C A); (2713 9780627 T C); (2714 9780892 T
C); (2715 9780968 A G); (2716 9781049 C T); (2717 9781056 C G); (2718 9781217 T C); (2719 9781251 A
G); (2720 9781262 G A); (2721 9781304 C T); (2722 9781321 T C); (2723 9781334 C T); (2724 9781522 C
T); (2725 9781653 T C); (2726 9781936 G T); (2727 9782081 G A); (2728 9782175 C T); (2729 9782223 C
T); (2730 9782359 T C); (2731 9782398 A G); (2732 9782604 A T); (2733 9782647 C A); (2734 9782651 C
T); (2735 9782774 T C); (2736 9782828 C T); (2737 9785297 C CA); (2738 9786079 T A); (2739 9786151 T
C); (2740 9786205 C T); (2741 9786299 T C); (2742 9786349 CT C); (2743 9786548 A G); (2744 9786616 A
C); (2745 9786691 G T); (2746 9786972 C G); (2747 9787053 C T); (2748 9787450 G A); (2749 9787587 A
C); (2750 9787603 G C); (2751 9787640 A G); (2752 9787826 T C); (2753 9787879 C CA); (2754 9788105 C
T); (2755 9788257 A G); (2756 9788358 C T); (2757 9788404 A G); (2758 9788426 A T); (2759 9788462 C
A); (2760 9788688 T C); (2761 9788964 G A); (2762 9789035 C T); (2763 9789443 T C); (2764 9789484 C
T); (2765 9789560 A G); (2766 9789638 G T); (2767 9790130 G T); (2768 9790195 A G); (2769 9790197 C
T); (2770 9790731 A G); (2771 9790749 C A); (2772 9790806 C A); (2773 9790843 T C); (2774 9790873 G
A); (2775 9790947 C T); (2776 9791258 G T); (2777 9791523 A G); (2778 9791730 G T); (2779 9791914 A
G); (2780 9791973 G A); (2781 9792129 A G); (2782 9792168 T C); (2783 9792175 T C); (2784 9792176 C
T); (2785 9792185 T C); (2786 9792259 A C); (2787 9792267 A G); (2788 9792369 T C); (2789 9792436 T
C); (2790 9792463 T C); (2791 9792598 C T); (2792 9792801 T C); (2793 9792878 A C); (2794 9793126 C
A); (2795 9793343 C T); (2796 9793569 A C); (2797 9793604 A AC); (2798 9793647 G A); (2799 9793660 C
A); (2800 9793879 C T); (2801 9794277 T C); (2802 9794279 G A); (2803 9794343 A G); (2804 9794394 C
A); (2805 9794813 AT A); (2806 9794873 T C); (2807 9794930 C T); (2808 9794983 A T); (2809 9795131 A
G); (2810 9795235 G A); (2811 9795779 T C); (2812 9795820 G A); (2813 9796041 C T); (2814 9796079
GA G); (2815 9796115 T C); (2816 9796155 C T); (2817 9796208 C CT); (2818 9796285 G A); (2819
9796449 C T); (2820 9796897 C T); (2821 9797014 G C); (2822 9797267 A G); (2823 9797558 C A); (2824
9798278 G A); (2825 9798439 G T); (2826 9798460 T A); (2827 9798562 T G); (2828 9798815 G C); (2829
9798947 A G); (2830 9799007 A G); (2831 9799320 C T); (2832 9799321 T C); (2833 9799332 C T); (2834
9799352 G T); (2835 9799502 G A); (2836 9799519 C T); (2837 9799722 G A); (2838 9799784 G A); (2839
9800018 C T); (2840 9800298 A C); (2841 9800342 T G); (2842 9800345 T C); (2843 9800424 T A); (2844
9800425 T G); (2845 9800494 C T); (2846 9800810 C T); (2847 9801035 T C); (2848 9801085 G T); (2849
9801115 A G); (2850 9801171 G A); (2851 9801385 T G); (2852 9801946 A G); (2853 9802093 C G); (2854
9802244 A T); (2855 9802544 G C); (2856 9802734 A C); (2857 9802793 C A); (2858 9802800 T A); (2859
9802976 T G); (2860 9803272 C T); (2861 9803377 C T); (2862 9803409 T C); (2863 9803766 AG A); (2864
9804039 T C); (2865 9804629 A T); (2866 9805156 G A); (2867 9805186 T A); (2868 9805349 G A); (2869
9805532 AG A); (2870 9805625 C A); (2871 9805717 G A); (2872 9805749 C T); (2873 9805792 T C); (2874
9806164 G A); (2875 9806265 G A); (2876 9806325 G A); (2877 9806392 C T); (2878 9806488 C T); (2879
9806867 A C); (2880 9806921 G A); (2881 9806993 A G); (2882 9807015 C T); (2883 9807197 C T); (2884
9807283 T C); (2885 9807380 C A); (2886 9807521 T C); (2887 9807626 G A); (2888 9807672 G T); (2889
9807815 T C); (2890 9808022 C T); (2891 9808296 T C); (2892 9808342 G A); (2893 9808403 T C); (2894
9808424 G A); (2895 9808444 T C); (2896 9808469 C T); (2897 9808496 A G); (2898 9808499 T C); (2899
9808551 A G); (2900 9808558 C G); (2901 9808569 G T); (2902 9808591 G A); (2903 9808608 C G); (2904
9808630 C T); (2905 9808665 C T); (2906 9808763 A G); (2907 9808901 T C); (2908 9809064 G A); (2909
9809248 G A); (2910 9809278 T C); (2911 9809309 T G); (2912 9809310 T G); (2913 9809326 A G); (2914
9809360 G T); (2915 9809377 T G); (2916 9809459 A C); (2917 9809588 A C); (2918 9809846 TC T); (2919
9810244 C T); (2920 9810812 A T); (2921 9810819 G T); (2922 9810948 C CAT); (2923 9811066 C
G); (2924 9811146 GAGCAA G); (2925 9811190 A G); (2926 9811262 T C); (2927 9811619 A C); (2928
9812024 T C); (2929 9812132 A C); (2930 9812225 A C); (2931 9812287 G GATGTTTA); (2932 9812432 C
T); (2933 9812458 T A); (2934 9812488 T C); (2935 9812518 C A); (2936 9812626 A G); (2937 9812793 G
A); (2938 9812956 C T); (2939 9813090 C T); (2940 9813109 G A); (2941 9813762 T C); (2942 9813848 G
A); (2943 9813867 G A); (2944 9813926 T A); (2945 9814081 G A); (2946 9814119 G A); (2947 9814130 T
C); (2948 9814451 A G); (2949 9814519 G T); (2950 9814598 T C); (2951 9814709 C T); (2952 9814817 A
G); (2953 9814885 A G); (2954 9814919 C T); (2955 9815550 G A); (2956 9815766 G A); (2957 9815823 G
A); (2958 9816196 C T); (2959 9816281 C T); (2960 9816360 T G); (2961 9816371 T A); (2962 9816414 T
A); (2963 9816608 T C); (2964 9816675 A C); (2965 9816729 A G); (2966 9817293 A C); (2967 9817356 A
C); (2968 9817433 A G); (2969 9817751 C A); (2970 9818623 C T); (2971 9818660 C T); (2972 9818672 C
A); (2973 9819066 C T); (2974 9819582 C T); (2975 9819583 A G); (2976 9819683 C T); (2977 9819919 T
C); (2978 9819975 C T); (2979 9820060 A G); (2980 9820599 T G); (2981 9820741 C A); (2982 9820904 A
G); (2983 9820907 A G); (2984 9821607 C A); (2985 9821879 A G); (2986 9822020 G A); (2987 9822107 A
G); (2988 9822276 C T); (2989 9822550 A G); (2990 9822633 T C); (2991 9822875 A T); (2992 9822924 G
A); (2993 9823488 C T); (2994 9823582 T A); (2995 9823585 G A); (2996 9823590 C A); (2997 9824288 G
A); (2998 9824379 G A); (2999 9824435 G A); (3000 9824636 A G); (3001 9824645 A G); (3002 9824723 A
G); (3003 9824814 G A); (3004 9824926 T G); (3005 9825287 G A); (3006 9825885 T C); (3007 9825977 A
G); (3008 9826254 C T); (3009 9826382 A G); (3010 9826415 A G); (3011 9826564 A C); (3012 9826593 G
A); (3013 9826605 G C); (3014 9826667 C A); (3015 9826693 A C); (3016 9826716 A T); (3017 9826759 A
T); (3018 9826786 A G); (3019 9826826 G A); (3020 9826868 G C); (3021 9826896 T G); (3022 9826900 G
A); (3023 9826931 G T); (3024 9826937 C A); (3025 9826979 C T); (3026 9827072 A G); (3027 9827074 A
G); (3028 9827132 A G); (3029 9827156 G T); (3030 9827182 G A); (3031 9827255 T C); (3032 9827398 A
G); (3033 9827400 T C); (3034 9827417 A C); (3035 9827439 A G); (3036 9827473 T C); (3037 9827475 T
C); (3038 9827719 G A); (3039 9827794 T A); (3040 9827807 T C); (3041 9827870 T C); (3042 9827890 G
A); (3043 9827986 G T); (3044 9828061 G A); (3045 9828570 G A); (3046 9828736 G A); (3047 9828855 T
A); (3048 9829045 C T); (3049 9829098 G A); (3050 9829265 C T); (3051 9829370 T A); (3052 9829373 G
T); (3053 9829619 TAAA T); (3054 9829800 G A); (3055 9829840 G T); (3056 9829984 C T); (3057
9830084 C T); (3058 9830166 C T); (3059 9830433 A C); (3060 9830508 A G); (3061 9830891 T C); (3062
9830943 T A); (3063 9830998 A G); (3064 9831089 T G); (3065 9831323 G A); (3066 9831454 G A); (3067
9831506 G A); (3068 9831609 T C); (3069 9831681 T C); (3070 9831767 T C); (3071 9831805 T C); (3072
9831902 T C); (3073 9832129 C G); (3074 9832601 G A); (3075 9832625 A G); (3076 9832834 C A); (3077
9832984 T C); (3078 9833602 G T); (3079 9834328 C T); (3080 9836536 C G); (3081 9836644 C T); (3082
9837562 G A); (3083 9841546 G A); (3084 9841692 G T); (3085 9841857 C T); (3086 9842190 G A); (3087
9843163 G A); (3088 9843175 C T); (3089 9843187 A G); (3090 9843219 T C); (3091 9843226 G A); (3092
9844163 G A); (3093 9844211 C T); (3094 9844220 T C); (3095 9844235 G C); (3096 9844249 A G); (3097
9845319 A G); (3098 9845375 G C); (3099 9845406 G T); (3100 9845453 C T); (3101 9845578 G A); (3102
9845589 T C); (3103 9845595 T C); (3104 9845596 A G); (3105 9845603 C A); (3106 9845686 C T); (3107
9845716 C T); (3108 9845725 G A); (3109 9846033 A G); (3110 9846051 G A); (3111 9846133 T G); (3112
9846193 T A); (3113 9846463 T TAA); (3114 9846547 A T); (3115 9846809 C T); (3116 9847168 G
A); (3117 9847275 A T); (3118 9847377 A G); (3119 9847581 A G); (3120 9847791 T C); (3121 9848747 C
T); (3122 9848905 T C); (3123 9849044 C T); (3124 9849048 C T); (3125 9849090 C T); (3126 9849155 TT
AA); (3127 9849195 G A); (3128 9849430 A G); (3129 9849531 C G); (3130 9850344 T C); (3131 9850431
A G); (3132 9850638 AT A); (3133 9850709 C G); (3134 9850832 C T); (3135 9850843 C T); (3136 9850899
T C); (3137 9850949 G A); (3138 9851121 T C); (3139 9851122 G A); (3140 9851130 C T); (3141 9851304
C A); (3142 9851357 G A); (3143 9851443 C A); (3144 9851460 A G); (3145 9851492 A G); (3146 9851523
G A); (3147 9851541 C G); (3148 9851605 A G); (3149 9851672 A G); (3150 9851696 A G); (3151 9851761
C T); (3152 9852000 C T); (3153 9852023 T C); (3154 9852049 C T); (3155 9852068 C T); (3156 9852093 T
C); (3157 9852905 C T); (3158 9852956 C A); (3159 9852998 T C); (3160 9853007 G A); (3161 9853123 T
C); (3162 9853181 C T); (3163 9853205 T C); (3164 9853208 C A); (3165 9853231 A G); (3166 9853237 G
A); (3167 9853239 T C); (3168 9853334 G T); (3169 9853397 A G); (3170 9853404 C T); (3171 9853408 C
T); (3172 9853736 G A); (3173 9854036 A C); (3174 9854055 C T); (3175 9854327 C T); (3176 9854441 C
T); (3177 9854600 A C); (3178 9854816 T C); (3179 9854885 G A); (3180 9854914 T C); (3181 9854964 T
C); (3182 9854979 C CG); (3183 9855002 T C); (3184 9855169 T A); (3185 9855239 G A); (3186 9855272 C
T); (3187 9855374 C T); (3188 9855403 G A); (3189 9855486 G GC); (3190 9855824 A G); (3191 9855975
C T); (3192 9856479 C T); (3193 9856494 G A); (3194 9856568 C T); (3195 9856574 T C); (3196 9856627 C
T); (3197 9856666 A T); (3198 9856709 A C); (3199 9857102 C T); (3200 9857288 T C); (3201 9857825 A
C); (3202 9857941 A G); (3203 9858163 T C); (3204 9858606 A AC); (3205 9858710 T G); (3206 9858902 G
T); (3207 9859326 T A); (3208 9859411 C A); (3209 9859610 G A); (3210 9859641 G A); (3211 9859871 C
A); (3212 9860012 G A); (3213 9860109 T C); (3214 9860142 A G); (3215 9860636 C T); (3216 9860685 T
C); (3217 9860896 C A); (3218 9860933 T G); (3219 9860956 A G); (3220 9860998 A G); (3221 9861062 G
T); (3222 9861131 A G); (3223 9861165 T C); (3224 9861300 G C); (3225 9861309 G T); (3226 9861447 A
G); (3227 9861497 T C); (3228 9861505 C T); (3229 9861570 T C); (3230 9861586 G A); (3231 9861653 T
C); (3232 9861740 G A); (3233 9861849 T C); (3234 9861954 T C); (3235 9862004 G A); (3236 9862031 T
C); (3237 9862067 G A); (3238 9862072 G A); (3239 9862181 G A); (3240 9862191 G A); (3241 9862239 G
GT); (3242 9862335 C T); (3243 9862618 T C); (3244 9862814 T C); (3245 9862855 T C); (3246 9863164 G
C); (3247 9863286 T C); (3248 9863391 G C); (3249 9863543 AAGAGCT A); (3250 9863794 G A); (3251
9863821 G C); (3252 9864139 T C); (3253 9864174 A G); (3254 9864208 T C); (3255 9864233 C T); (3256
9864533 AT A); (3257 9864628 T C); (3258 9864967 C T); (3259 9864978 GA G); (3260 9865086 A
G); (3261 9865198 C T); (3262 9865203 T C); (3263 9865337 C T); (3264 9865340 G A); (3265 9865349 A
C); (3266 9865437 C T); (3267 9865459 T C); (3268 9865465 T A); (3269 9865469 T C); (3270 9865563 T
C); (3271 9865658 T TA); (3272 9866004 A T); (3273 9866081 T G); (3274 9866292 T TA); (3275 9866808
A G); (3276 9867099 G A); (3277 9868338 G A); (3278 9868372 TTAATACAA T); (3279 9868487 C
A); (3280 9868591 C T); (3281 9868771 G A); (3282 9868790 A T); (3283 9868854 G C); (3284 9868987 C
A); (3285 9869259 T A); (3286 9869261 AT A); (3287 9869505 A C); (3288 9869545 T G); (3289 9869573 G
T); (3290 9869936 T C); (3291 9869999 A AT); (3292 9870073 GT G); (3293 9870188 C A); (3294 9870204
T C); (3295 9870277 A G); (3296 9870279 G C); (3297 9870381 C G); (3298 9870578 T C); (3299 9870795
C A); (3300 9870928 C T); (3301 9870996 A G); (3302 9871299 T A); (3303 9871451 A T); (3304 9871506
G A); (3305 9871613 T C); (3306 9871669 G A); (3307 9871792 T C); (3308 9871818 A G); (3309 9872014
G A); (3310 9872030 T G); (3311 9872223 A G); (3312 9872291 A G); (3313 9872325 G A); (3314 9872367
A G); (3315 9872415 C T); (3316 9872444 C A); (3317 9872512 AT A); (3318 9872539 T C); (3319 9872585
T C); (3320 9872641 A G); (3321 9872757 G A); (3322 9872775 G A); (3323 9873097 A G); (3324 9873375
A T); (3325 9873519 T C); (3326 9873562 C T); (3327 9873602 A G); (3328 9873778 G A); (3329 9874260
G A); (3330 9874350 C T); (3331 9874471 G A); (3332 9874503 A G); (3333 9874520 A G); (3334 9874652
G A); (3335 9874677 A G); (3336 9874685 T C); (3337 9874699 C T); (3338 9874820 T C); (3339 9874889 T
C); (3340 9875045 A G); (3341 9875048 T C); (3342 9875107 A G); (3343 9875359 A G); (3344 9875470 A
G); (3345 9875477 TA T); (3346 9875550 G A); (3347 9875600 G T); (3348 9875606 T C); (3349 9875685 A
C); (3350 9875753 C T); (3351 9875851 C A); (3352 9876086 C T); (3353 9876189 A G); (3354 9876199 A
G); (3355 9876211 T C); (3356 9876256 C T); (3357 9876451 A G); (3358 9876555 A G); (3359 9876568 A
AT); (3360 9876703 T C); (3361 9876726 G A); (3362 9876737 A G); (3363 9876754 A G); (3364 9876803
A G); (3365 9876886 G A); (3366 9876999 C T); (3367 9877028 C T); (3368 9877103 A T); (3369 9877298
C T); (3370 9877327 A G); (3371 9877429 G C); (3372 9877431 T C); (3373 9877435 A G); (3374 9877446
A G); (3375 9877552 T C); (3376 9877570 G A); (3377 9877969 G T); (3378 9878023 C T); (3379 9878081
C T); (3380 9878082 A G); (3381 9878246 G A); (3382 9878263 T C); (3383 9878301 C A); (3384 9878351
A G); (3385 9878364 T A); (3386 9878665 A G); (3387 9878698 T C); (3388 9878859 A C); (3389 9878916
T C); (3390 9879047 T C); (3391 9879061 G A); (3392 9879138 A G); (3393 9879139 A T); (3394 9879232
T A); (3395 9879362 G A); (3396 9879438 C T); (3397 9879450 T C); (3398 9879470 T G); (3399 9879482
A G); (3400 9879484 C T); (3401 9879496 A G); (3402 9879572 T C); (3403 9879979 T C); (3404 9880477
G A); (3405 9880505 C T); (3406 9880601 T C); (3407 9880685 C T); (3408 9880695 C T); (3409 9880737 A
G); (3410 9880747 T C); (3411 9880830 T G); (3412 9880910 A G); (3413 9880927 T G); (3414 9880943 G
T); (3415 9881025 G A); (3416 9882564 A G); (3417 9882658 A G); (3418 9882741 G A); (3419 9882755 G
T); (3420 9882898 T C); (3421 9883078 C T); (3422 9883089 CT C); (3423 9883526 A G); (3424 9883661 A
G); (3425 9883784 T C); (3426 9883789 G C); (3427 9883803 A G); (3428 9884181 G A); (3429 9884183 A
C); (3430 9884440 A G); (3431 9885028 G A); (3432 9885326 G A); (3433 9885338 A G); (3434 9885519 G
A); (3435 9885598 G T); (3436 9885742 A G); (3437 9885860 T C); (3438 9886606 A G); (3439 9886788 A
T); (3440 9887193 A G); (3441 9887488 A G); (3442 9887669 T G); (3443 9887897 A G); (3444 9888383 T
G); (3445 9888430 G A); (3446 9888529 C T); (3447 9888530 G A); (3448 9888572 A G); (3449 9888651 A
G); (3450 9888685 G A); (3451 9888698 A G); (3452 9888897 G A); (3453 9888936 T C); (3454 9889109 A
C); (3455 9889680 A G); (3456 9889906 G A); (3457 9890183 T C); (3458 9890409 A G); (3459 9890441 A
T); (3460 9890795 C T); (3461 9890856 C T); (3462 9890877 GTGA G); (3463 9890881 T C); (3464
9890892 G A); (3465 9890976 T C); (3466 9891033 A G); (3467 9891118 T C); (3468 9891201 T C); (3469
9891279 G A); (3470 9891424 G T); (3471 9891484 T C); (3472 9891498 G A); (3473 9891528 C T); (3474
9891590 C T); (3475 9891826 G T); (3476 9891827 C T); (3477 9891850 G T); (3478 9891960 C T); (3479
9891995 G A); (3480 9892173 G A); (3481 9892236 A G); (3482 9892404 A G); (3483 9892521 G A); (3484
9892553 A G); (3485 9892581 C T); (3486 9892592 T C); (3487 9892629 G A); (3488 9892763 T C); (3489
9892787 C T); (3490 9893054 G A); (3491 9893065 T C); (3492 9893108 A G); (3493 9893132 G A); (3494
9893161 T C); (3495 9893239 A G); (3496 9893272 G A); (3497 9893283 G T); (3498 9893327 T G); (3499
9893359 G A); (3500 9893457 C A); (3501 9893732 A G); (3502 9893972 G A); (3503 9894095 G A); (3504
9894315 G A); (3505 9894411 A G); (3506 9894489 C T); (3507 9894619 A G); (3508 9895052 C T); (3509
9895330 A G); (3510 9895424 G A); (3511 9895458 A G); (3512 9895578 T C); (3513 9895595 CG
C); (3514 9895671 C T); (3515 9896134 T C); (3516 9896333 G A); (3517 9897167 C T); (3518 9897254 C
A); (3519 9897524 A G); (3520 9897866 A G); (3521 9898720 C A); (3522 9898843 A C); (3523 9898926 T
TAC); (3524 9898933 C A); (3525 9899029 A C); (3526 9899056 C T); (3527 9899064 G T); (3528 9899076
A G); (3529 9899135 C T); (3530 9899161 T C); (3531 9899162 A G); (3532 9899172 A G); (3533 9900237
A G); (3534 9900282 T C); (3535 9900387 T C); (3536 9900434 A G); (3537 9900931 T A); (3538 9901519
T C); (3539 9901560 A G); (3540 9901759 T C); (3541 9901823 A G); (3542 9902035 T C); (3543 9902136 T
C); (3544 9902152 T TACGA); (3545 9902241 G A); (3546 9902373 T A); (3547 9902388 TA T); (3548
9902412 T G); (3549 9902731 C G); (3550 9903060 G GA); (3551 9903113 C T); (3552 9903289 A
G); (3553 9903296 G T); (3554 9903390 C A); (3555 9903475 C A); (3556 9903479 GA G); (3557 9903587
G A); (3558 9903618 G A); (3559 9903971 C T); (3560 9904003 A G); (3561 9904370 A AC); (3562
9904583 A C); (3563 9905036 C T); (3564 9905190 T C); (3565 9905530 G A); (3566 9905558 T
TGTTTTTGAA); (3567 9905571 T G); (3568 9905725 T G); (3569 9905799 G A); (3570 9905809 A G); (3571
9905883 G A); (3572 9905931 C T); (3573 9905998 C T); (3574 9906021 G A); (3575 9906084 A G); (3576
9906100 C A); (3577 9906218 G A); (3578 9906414 G A); (3579 9906654 T C); (3580 9906669 TA T); (3581
9907725 T C); (3582 9907811 AC A); (3583 9907875 A G); (3584 9908117 G A); (3585 9908249 G
A); (3586 9908265 T G); (3587 9908315 A G); (3588 9908692 A T); (3589 9908722 T C); (3590 9908771 G
A); (3591 9908815 C T); (3592 9908933 C T); (3593 9909070 T C); (3594 9909099 C T); (3595 9909227 A
G); (3596 9909339 G C); (3597 9909387 G A); (3598 9909591 A G); (3599 9910286 G C); (3600 9910499 T
C); (3601 9910665 G A); (3602 9910696 GT G); (3603 9910821 T G); (3604 9911261 G A); (3605 9911515
A C); (3606 9912168 T C); (3607 9912192 A G); (3608 9912198 G A); (3609 9912208 T C); (3610 9912455
A G); (3611 9912486 A G); (3612 9912505 G A); (3613 9912511 A G); (3614 9912554 T C); (3615 9912603
A G); (3616 9912680 C T); (3617 9912698 T C); (3618 9912748 G C); (3619 9912788 C T); (3620 9912847
A G); (3621 9912959 C T); (3622 9913175 A G); (3623 9913278 A G); (3624 9913306 T C); (3625 9913476
A G); (3626 9913737 A G); (3627 9913738 A C); (3628 9913784 A G); (3629 9914091 A G); (3630 9914143
A G); (3631 9914179 A G); (3632 9914283 G A); (3633 9914288 A G); (3634 9914433 A G); (3635 9914445
C T); (3636 9914455 T C); (3637 9914630 A G); (3638 9914706 C T); (3639 9914715 C T); (3640 9914723 G
A); (3641 9914764 T C); (3642 9914843 T G); (3643 9914865 A G); (3644 9914988 T C); (3645 9915110 T
A); (3646 9915477 G A); (3647 9915480 T G); (3648 9915485 T C); (3649 9915699 G T); (3650 9915887 G
A); (3651 9916562 G A); (3652 9916666 A G); (3653 9916668 C T); (3654 9917066 C T); (3655 9917206 A
C); (3656 9917348 G A); (3657 9917355 G T); (3658 9917369 G A); (3659 9917655 G A); (3660 9917762 T
A); (3661 9917879 A G); (3662 9918216 T C); (3663 9918470 A G); (3664 9918725 G A); (3665 9918902 T
C); (3666 9918935 G A); (3667 9918994 T C); (3668 9919069 A G); (3669 9919269 C T); (3670 9920109 T
C); (3671 9920590 A G); (3672 9920698 A G); (3673 9920834 C T); (3674 9921059 T C); (3675 9921079 G
A); (3676 9921115 G A); (3677 9921226 T C); (3678 9921275 A G); (3679 9921854 G A); (3680 9921975 T
G); (3681 9922069 T C); (3682 9922139 T C); (3683 9922394 A C); (3684 9922457 T C); (3685 9922525 T
C); (3686 9922569 C A); (3687 9922686 T C); (3688 9922717 G A); (3689 9922908 T C); (3690 9922962 A
G); (3691 9923309 A T); (3692 9923463 G A); (3693 9923509 C CT); (3694 9923948 T C); (3695 9924577 G
A); (3696 9925033 T C); (3697 9925391 A G); (3698 9925524 C T); (3699 9925625 G C); (3700 9925734 A
G); (3701 9925738 A T); (3702 9925767 T C); (3703 9926198 G A); (3704 9926476 C T); (3705 9926600 T
C); (3706 9926777 G A); (3707 9926946 A G); (3708 9927015 T C); (3709 9927031 T G); (3710 9927175 T
C); (3711 9927387 T C); (3712 9927435 A G); (3713 9927625 T C); (3714 9927711 T C); (3715 9927724 G
A); (3716 9927981 T A); (3717 9928077 T C); (3718 9928123 G A); (3719 9928152 A C); (3720 9928153 G
A); (3721 9928154 T C); (3722 9928155 G T); (3723 9928317 A C); (3724 9928325 C T); (3725 9928657 C
T); (3726 9928750 C T); (3727 9928858 A G); (3728 9928955 C A); (3729 9928974 A C); (3730 9929121 A
T); (3731 9929240 T C); (3732 9929274 C T); (3733 9929420 A G); (3734 9929491 A G); (3735 9929508
ACCC A); (3736 9929757 C G); (3737 9929791 G A); (3738 9929862 G A); (3739 9930019 A G); (3740
9930100 T A); (3741 9930119 C T); (3742 9930124 A G); (3743 9930125 G C); (3744 9930126 C T); (3745
9930165 G GA); (3746 9930189 A G); (3747 9930213 A T); (3748 9930245 G A); (3749 9930273 GA
G); (3750 9930503 A G); (3751 9931046 T C); (3752 9931102 T G); (3753 9931390 A G); (3754 9931401 T
C); (3755 9931433 C A); (3756 9931437 G A); (3757 9931460 T C); (3758 9931761 A G); (3759 9931871 T
C); (3760 9932054 T C); (3761 9932294 C T); (3762 9932592 C T); (3763 9932719 G A); (3764 9932738 C
T); (3765 9932815 T A); (3766 9933143 G A); (3767 9935529 T C); (3768 9937601 G A); (3769 9937905 T
A); (3770 9938288 G A); (3771 9938493 C G); (3772 9938553 T C); (3773 9938715 C T); (3774 9938930 T
C); (3775 9939035 C A); (3776 9939296 C A); (3777 9939396 A G); (3778 9939638 T C); (3779 9939650 G
A); (3780 9939678 C A); (3781 9939838 C T); (3782 9940006 C A); (3783 9940114 C T); (3784 9940171 A
C); (3785 9940211 A T); (3786 9940319 G A); (3787 9940325 A G); (3788 9940327 T G); (3789 9940413 T
C); (3790 9940420 A G); (3791 9940450 T A); (3792 9940481 T A); (3793 9940587 T C); (3794 9941272 C
T); (3795 9941367 A G); (3796 9941776 A C); (3797 9941805 G T); (3798 9941918 C T); (3799 9942111 T
C); (3800 9942219 A G); (3801 9942404 A G); (3802 9943103 G A); (3803 9943180 C T); (3804 9943239 A
G); (3805 9943449 A T); (3806 9943521 A G); (3807 9943535 T G); (3808 9943560 A G); (3809 9943615 A
C); (3810 9943618 G A); (3811 9944209 G A); (3812 9944334 G A); (3813 9944396 T C); (3814 9944408 C
G); (3815 9944494 C T); (3816 9944565 C T); (3817 9944834 G A); (3818 9944960 T C); (3819 9945158 T
C); (3820 9945623 G A); (3821 9945742 A G); (3822 9945758 A G); (3823 9945774 TTG T); (3824 9945807
G A); (3825 9946109 A G); (3826 9946207 A T); (3827 9946246 T A); (3828 9946328 G A); (3829 9946332
A T); (3830 9946402 G A); (3831 9946409 C T); (3832 9946774 T G); (3833 9947270 G C); (3834 9947455
T A); (3835 9947465 A G); (3836 9947528 A G); (3837 9947582 CA C); (3838 9948002 T G); (3839
9948235 CT C); (3840 9948816 C T); (3841 9948927 G T); (3842 9949058 A G); (3843 9949324 C T); (3844
9950465 A G); (3845 9950682 G T); (3846 9950858 C T); (3847 9951041 T C); (3848 9951374 T C); (3849
9951588 C T); (3850 9951619 A G); (3851 9951641 T G); (3852 9952034 T C); (3853 9952244 G A); (3854
9952472 G C); (3855 9953284 C T); (3856 9953346 C T); (3857 9953356 G A); (3858 9953386 C T); (3859
9953709 T TG); (3860 9954093 C A); (3861 9954174 C T); (3862 9954191 T C); (3863 9955211 G T); (3864
9960674 C G); (3865 9961405 A G); (3866 9961697 C T); (3867 9961993 A G); (3868 9962057 A T); (3869
9962241 C T); (3870 9962252 T C); (3871 9962637 T A); (3872 9963980 G T); (3873 9964360 AT A); (3874
9964369 C T); (3875 9964550 G A); (3876 9964595 T C); (3877 9964712 A C); (3878 9964741 A G); (3879
9965412 A G); (3880 9965530 G A); (3881 9965713 T A); (3882 9965849 T G); (3883 9966890 G T); (3884
9967200 G A); (3885 9967519 G A); (3886 9967528 A AAGGTG); (3887 9968097 G C); (3888 9968304 A
G); (3889 9968306 G A); (3890 9968310 A G); (3891 9968349 T A); (3892 9968642 A G); (3893 9969360
AG A); (3894 9969430 G C); (3895 9969527 T TA); (3896 9973835 T A); (3897 9974181 A G); (3898
9974474 C T); (3899 9974728 A G); (3900 9974770 C T); (3901 9975289 G A); (3902 9975477 A G); (3903
9975478 C T); (3904 9975528 G A); (3905 9975784 C T); (3906 9976194 C T); (3907 9976237 C T); (3908
9976253 GCAACTTTTGGCAACA G); (3909 9976347 T G); (3910 9977196 G A); (3911 9977551 A G); (3912
9978449 G A); (3913 9979145 T C); (3914 9979171 T C); (3915 9979232 A C); (3916 9979377 C T); (3917
9979821 C G); (3918 9980013 A T); (3919 9980369 A T); (3920 9981001 C T); (3921 9981095 G A); (3922
9981139 A G); (3923 9981251 A G); (3924 9981380 C T); (3925 9981385 C T); (3926 9981781 C T); (3927
9982256 A AG); (3928 9982469 A G); (3929 9982606 C G); (3930 9982763 T C); (3931 9983262 A
G); (3932 9983560 G C); (3933 9983591 G A); (3934 9983809 T G); (3935 9983856 T C); (3936 9984336 A
G); (3937 9984558 G T); (3938 9984639 C T); (3939 9985324 C T); (3940 9985467 C T); (3941 9985875 T
G); (3942 9986240 T C); (3943 9986710 C T); (3944 9986714 G A); (3945 9986721 A C); (3946 9987822 G
A); (3947 9987863 T A); (3948 9987883 A G); (3949 9988484 A G); (3950 9988993 G A); (3951 9989478 T
C); (3952 9990366 T C); (3953 9990917 C T); (3954 9991022 C A); (3955 9991164 A AG); (3956 9991206 A
G); (3957 9991254 A G); (3958 9991328 T C); (3959 9991900 G A); (3960 9991938 T C); (3961 9992163 G
A); (3962 9992893 G C); (3963 9993582 T C); (3964 9993843 C T); (3965 9993959 T C); (3966 9994102 G
A); (3967 9994222 C T); (3968 9994255 C T); (3969 9994675 A C); (3970 10004835 G A); (3971 10004895
A G); (3972 10005182 G A); (3973 10005221 G A); (3974 10005443 C T); (3975 10005953 A G); (3976
10006306 A G); (3977 10006308 C T); (3978 10006468 G A); (3979 10006765 G T); (3980 10007105 A
G); (3981 10007301 C T); (3982 10007828 G A); (3983 10007888 C A); (3984 10008071 G T); (3985
10008094 G A); (3986 10008238 T C); (3987 10008717 A G); (3988 10009156 G A); (3989 10009892 T
C); (3990 10010699 T C); (3991 10010904 A G); (3992 10010960 T C); (3993 10011197 C T); (3994
10011566 A G); (3995 10011574 G T); (3996 10011634 A G); (3997 10012253 T C); (3998 10012341 G
A); (3999 10012386 A G); (4000 10012660 G C); (4001 10013472 G T); (4002 10013582 G A); (4003
10013744 T G); (4004 10013846 G T); (4005 10013958 A T); (4006 10014019 G A); (4007 10014357 A
C); (4008 10014469 A G); (4009 10014597 T G); (4010 10015872 C A); (4011 10016062 C T); (4012
10016491 A C); (4013 10016721 C T); (4014 10016751 A G); (4015 10016879 C T); (4016 10016883 A
G); (4017 10017093 T C); (4018 10017166 A G); (4019 10017217 G A); (4020 10017253 T C); (4021
10018002 G A); (4022 10018081 G A); (4023 10018452 T C); (4024 10018461 A G); (4025 10018539 A
G); (4026 10018545 G T); (4027 10018551 G A); (4028 10018581 C T); (4029 10018590 C T); (4030
10018828 A G); (4031 10019076 G A); (4032 10019111 T C); (4033 10019127 C T); (4034 10019821 T
C); (4035 10020301 T C); (4036 10020504 A G); (4037 10020700 C T); (4038 10020833 A G); (4039
10021015 A G); (4040 10021186 C T); (4041 10021187 A G); (4042 10021203 A AT); (4043 10021657 G
A); (4044 10021664 C T); (4045 10022082 A T); (4046 10022124 G C); (4047 10022967 T C); (4048
10023821 G T); (4049 10023952 G A); (4050 10023956 T C); (4051 10024003 A G); (4052 10024227 G
A); (4053 10024331 T C); (4054 10024489 A G); (4055 10024510 A C); (4056 10025699 T C); (4057
10026639 T G); (4058 10027233 G A); (4059 10027551 G C); (4060 10027847 A T); (4061 10028076 T
A); (4062 10028082 C T); (4063 10028290 T C); (4064 10029886 C T); (4065 10030086 T A); (4066
10030409 T A); (4067 10030748 C T); (4068 10030850 G A); (4069 10030946 C T); (4070 10031562 A
T); (4071 10031826 C T); (4072 10031859 T G); (4073 10032867 A G); (4074 10033301 A C); (4075
10033750 A C); (4076 10033756 A G); (4077 10034385 T G); (4078 10034895 G T); (4079 10034926 C
T); (4080 10036079 A G); (4081 10036267 A G); (4082 10036269 G A); (4083 10036283 C T); (4084
10036284 A G); (4085 10036333 C CA); (4086 10036379 G A); (4087 10036442 T C); (4088 10037382 T
TA); (4089 10037891 T A); (4090 10038900 T C); (4091 10040185 C A); (4092 10040535 A C); (4093
10040806 A G); (4094 10040991 C G); (4095 10041798 C G); (4096 10042843 T C); (4097 10043955 CT
C); (4098 10044388 A G); (4099 10044537 C T); (4100 10045127 AT A); (4101 10045162 T TA); (4102
10045290 G A); (4103 10045361 G T); (4104 10045739 C T); (4105 10046273 C T); (4106 10046413 G
A); (4107 10046416 G A); (4108 10046421 C T); (4109 10046604 C T); (4110 10046805 T C); (4111
10046903 T C); (4112 10046940 A G); (4113 10047874 C T); (4114 10048486 T G); (4115 10048931 A
G); (4116 10049774 T C); (4117 10049788 G A); (4118 10050487 C T); (4119 10050608 T C); (4120
10050802 G A); (4121 10051024 C T); (4122 10051175 G T); (4123 10051286 A G); (4124 10051307 A
G); (4125 10051393 T G); (4126 10051481 C T); (4127 10051871 C T); (4128 10051916 A T); (4129
10052086 A G); (4130 10052527 A G); (4131 10052671 G T); (4132 10052715 G A); (4133 10052735 G
T); (4134 10052878 T A); (4135 10053591 A G); (4136 10053595 C T); (4137 10054478 T C); (4138
10054531 G A); (4139 10055199 G A); (4140 10055830 C T); (4141 10056012 A ATTATCTATCTT); (4142
10056308 C G); (4143 10056735 G A); (4144 10056825 T C); (4145 10056912 C A); (4146 10056972 G
A); (4147 10057072 G C); (4148 10057124 C T); (4149 10058310 A G); (4150 10058923 T C); (4151
10061737 C CT); (4152 10061964 T A); (4153 10061989 G A); (4154 10062027 G A); (4155 10062090 G
A); (4156 10062512 A G); (4157 10062529 A G); (4158 10063839 T C); (4159 10064510 G T); (4160
10064774 G A); (4161 10065555 A G); (4162 10065568 A T); (4163 10065727 T G); (4164 10065804 T
C); (4165 10065887 C T); (4166 10067105 C T); (4167 10067146 T C); (4168 10067826 TA T); (4169
10068365 C T); (4170 10069045 A G); (4171 10069943 T TCAGG); (4172 10070284 G T); (4173 10070400
A G); (4174 10070518 G A); (4175 10071616 G A); (4176 10071739 T G); (4177 10071864 G A); (4178
10071931 TA T); (4179 10072931 A G); (4180 10073443 T A); (4181 10073983 G A); (4182 10074096 T
C); (4183 10074410 T C); (4184 10074721 T C); (4185 10074823 G A); (4186 10074996 C T); (4187
10074999 C T); (4188 10075344 T C); (4189 10075652 A G); (4190 10077388 T C); (4191 10079298 A
G); (4192 10079901 A G); (4193 10080933 C T); (4194 10081328 T C); (4195 10081410 G A); (4196
10082486 T C); (4197 10082575 G A); (4198 10083027 G T); (4199 10083052 T C); (4200 10083901 T
C); (4201 10083942 C G); (4202 10084430 C T); (4203 10084501 T C); (4204 10084921 T C); (4205
10085379 C A); (4206 10085949 C T); (4207 10086422 T C); (4208 10087025 C G); (4209 10087625 G
A); (4210 10087655 C T); (4211 10087674 G A); (4212 10087693 C T); (4213 10087759 T C); (4214
10087761 C T); (4215 10087973 T G); (4216 10088261 C T); (4217 10088410 C T); (4218 10088944 G
A); (4219 10090036 T C); (4220 10090206 A G); (4221 10090671 A G); (4222 10091409 T C); (4223
10092226 T A); (4224 10092370 T C); (4225 10093615 C T); (4226 10093774 A G); (4227 10093893 T
C); (4228 10093908 A T); (4229 10094020 C T); (4230 10094169 C CG); (4231 10094571 C T); (4232
10094854 A G); (4233 10096086 C T); (4234 10096148 G A); (4235 10096402 T C); (4236 10096961 G
A); (4237 10097227 C T); (4238 10097337 T C); (4239 10097382 C T); (4240 10097436 C T); (4241
10097500 T G); (4242 10097513 G C); (4243 10097537 C T); (4244 10097669 T C); (4245 10097831 T
G); (4246 10097983 A T); (4247 10098092 T C); (4248 10098106 A C); (4249 10098433 A G); (4250
10098685 G A); (4251 10098744 T C); (4252 10098762 T C); (4253 10099010 A G); (4254 10099337 G
A); (4255 10099526 T C); (4256 10099655 T TTGGATGTG); (4257 10099738 C A); (4258 10099752 T
C); (4259 10100328 A G); (4260 10100358 G A); (4261 10100921 G T); (4262 10101583 A G); (4263
10101632 G A); (4264 10102482 T G); (4265 10102630 T C); (4266 10102932 G A); (4267 10103239 C
CTT); (4268 10103286 T C); (4269 10103367 T C); (4270 10103405 T A); (4271 10103407 T A); (4272
10103409 T A); (4273 10103417 T TTA); (4274 10103420 C T); (4275 10103978 C T); (4276 10104212 G
T); (4277 10104589 C G); (4278 10104636 A C); (4279 10105983 A AT); (4280 10106219 A G); (4281
10107331 C A); (4282 10107632 A AT); (4283 10107740 T G); (4284 10107982 G A); (4285 10108364 A
G); (4286 10108492 A T); (4287 10108590 T C); (4288 10108618 T C); (4289 10108664 T C); (4290
10108683 C T); (4291 10108929 C T); (4292 10109447 G A); (4293 10109462 T A); (4294 10111136 A
G); (4295 10111177 A T); (4296 10111307 G A); (4297 10111349 AT A); (4298 10111693 G A); (4299
10112093 G T); (4300 10112215 A G); (4301 10112304 G A); (4302 10112375 G A); (4303 10112638 T
C); (4304 10112784 CA C); (4305 10112845 A C); (4306 10113055 TAA T); (4307 10113155 A AAT); (4308
10113206 A G); (4309 10114307 A G); (4310 10114463 A G); (4311 10114629 G A); (4312 10114937 C
T); (4313 10115696 G A); (4314 10116329 T C); (4315 10116379 G A); (4316 10116380 G A); (4317
10116627 T C); (4318 10116808 A G); (4319 10116985 T C); (4320 10117060 A G); (4321 10117132 T
G); (4322 10117154 A G); (4323 10117746 G A); (4324 10118652 G T); (4325 10118749 A G); (4326
10119495 T C); (4327 10119846 T G); (4328 10119978 C G); (4329 10120366 A G); (4330 10120512 A
G); (4331 10120699 T C); (4332 10120864 T C); (4333 10121466 A G); (4334 10121589 G A); (4335
10121791 C A); (4336 10121803 A T); (4337 10122080 C T); (4338 10122092 A G); (4339 10122119 G
T); (4340 10122227 T C); (4341 10122239 C T); (4342 10122408 T A); (4343 10122437 C T); (4344
10124209 T C); (4345 10124597 G A); (4346 10124602 T A); (4347 10125679 G T); (4348 10126825 C
T); (4349 10127307 A T); (4350 10127823 A AT); (4351 10128142 G A); (4352 10128403 A C); (4353
10128468 A T); (4354 10128514 G A); (4355 10128548 G A); (4356 10128796 A G); (4357 10129033 T
C); (4358 10129213 C T); (4359 10129263 A G); (4360 10129342 G A); (4361 10129459 G A); (4362
10129492 C CTG); (4363 10129837 A AAT); (4364 10130006 C T); (4365 10130099 C A); (4366 10130338
A G); (4367 10130372 G A); (4368 10130665 G A); (4369 10130804 C T); (4370 10131119 C T); (4371
10131250 G A); (4372 10131317 T C); (4373 10131417 C T); (4374 10131726 T C); (4375 10131796 C
T); (4376 10131842 G A); (4377 10131914 T C); (4378 10132155 G A); (4379 10132246 A G); (4380
10132366 G A); (4381 10132472 A C); (4382 10132576 C G); (4383 10132590 C T); (4384 10132661 G
A); (4385 10132704 A G); (4386 10132986 C T); (4387 10132999 C T); (4388 10133070 G A); (4389
10133188 T C); (4390 10133320 A C); (4391 10133823 G T); (4392 10133915 T TTTTA); (4393 10134043 T
C); (4394 10134453 T C); (4395 10134492 G A); (4396 10134908 GT G); (4397 10134993 T A); (4398
10135584 T G); (4399 10135617 C CTAGA); (4400 10136138 G A); (4401 10136188 G A); (4402 10136201
G A); (4403 10136240 A G); (4404 10136299 A G); (4405 10136470 G A); (4406 10137297 C T); (4407
10137302 G A); (4408 10137365 T G); (4409 10137487 T C); (4410 10137617 A G); (4411 10137716 C
T); (4412 10137806 C T); (4413 10138573 C CACGTGG); (4414 10138612 G A); (4415 10138667 G
A); (4416 10138774 A T); (4417 10138803 T C); (4418 10139527 TA T); (4419 10139669 AT A); (4420
10140391 A T); (4421 10140491 A C); (4422 10142202 G C); (4423 10142459 G C); (4424 10142796 C
T); (4425 10142852 C T); (4426 10143253 T C); (4427 10143465 G A); (4428 10143666 A T); (4429
10144143 T C); (4430 10144731 T C); (4431 10144773 C A); (4432 10145171 C T); (4433 10145331 G
A); (4434 10145454 T C); (4435 10145811 TGAAGCTTCC T); (4436 10145825 G A); (4437 10145900 C
T); (4438 10146000 C T); (4439 10146109 C T); (4440 10146461 A G); (4441 10146508 TATAACC T); (4442
10146587 C A); (4443 10146638 C A); (4444 10147277 T C); (4445 10147472 A G); (4446 10147489 T
C); (4447 10147594 A G); (4448 10147654 G A); (4449 10147990 T G); (4450 10148018 G A); (4451
10148071 G A); (4452 10148386 G A); (4453 10148891 T C); (4454 10149197 G A); (4455 10149295 C
T); (4456 10149433 T C); (4457 10149527 C T); (4458 10149651 G C); (4459 10149910 A C); (4460
10149964 A G); (4461 10150154 A G); (4462 10151050 A G); (4463 10151187 C A); (4464 10151784 T
C); (4465 10151931 T G); (4466 10152030 A G); (4467 10152367 C G); (4468 10152422 G A); (4469
10152467 A G); (4470 10152480 T C); (4471 10152513 C T); (4472 10152565 C G); (4473 10152652 A
G); (4474 10152728 A T); (4475 10152828 C T); (4476 10152991 T C); (4477 10153058 C G); (4478
10153163 G A); (4479 10153202 G A); (4480 10153554 A G); (4481 10153693 A G); (4482 10153766 G
T); (4483 10154058 T A); (4484 10154455 C G); (4485 10155043 GT G); (4486 10155154 C T); (4487
10155210 G A); (4488 10155337 C T); (4489 10155860 C T); (4490 10155991 G T); (4491 10156263 G
A); (4492 10156366 G A); (4493 10156469 G A); (4494 10156790 T C); (4495 10156794 G A); (4496
10157139 C G); (4497 10157183 T A); (4498 10157213 A G); (4499 10157256 T C); (4500 10157474 C
T); (4501 10158699 C G); (4502 10158834 C A); (4503 10160281 C CA); (4504 10161130 C T); (4505
10161401 T A); (4506 10161516 TTA T); (4507 10161976 A G); (4508 10161977 G A); (4509 10163392 C
T); (4510 10163449 T G); (4511 10163732 C A); (4512 10163763 A G); (4513 10164387 A G); (4514
10164451 G A); (4515 10164650 T C); (4516 10164802 G A); (4517 10165422 C G); (4518 10165518 T
A); (4519 10165646 G T); (4520 10165686 G A); (4521 10165731 T C); (4522 10165746 T C); (4523
10165941 G A); (4524 10166394 A G); (4525 10166509 T C); (4526 10166532 G A); (4527 10166559 A
G); (4528 10166603 A T); (4529 10166652 G A); (4530 10166764 A C); (4531 10166808 G A); (4532
10167393 T C); (4533 10167704 T G); (4534 10168012 G C); (4535 10168017 G GAC); (4536 10168928 C
T); (4537 10168939 A G); (4538 10169661 C A); (4539 10169949 G T); (4540 10170056 T A); (4541
10170348 C T); (4542 10170440 C A); (4543 10170632 C T); (4544 10170636 G A); (4545 10170807 G
A); (4546 10170829 G T); (4547 10170966 G A); (4548 10171056 T C); (4549 10171621 C T); (4550
10171686 T C); (4551 10172344 C T); (4552 10172463 G A); (4553 10172617 G C); (4554 10172755 C
T); (4555 10173128 A G); (4556 10173323 C A); (4557 10173333 A G); (4558 10173428 T C); (4559
10173530 T C); (4560 10173881 T C); (4561 10173927 C T); (4562 10174019 C T); (4563 10174182 G
A); (4564 10174393 G A); (4565 10174914 G A); (4566 10175148 C T); (4567 10175188 G A); (4568
10175389 A G); (4569 10175658 G A); (4570 10176092 G A); (4571 10176405 G A); (4572 10176700 C
T); (4573 10176892 A G); (4574 10176981 T C); (4575 10177025 G A); (4576 10177263 T TA); (4577
10177733 G A); (4578 10178559 T TA); (4579 10178624 T C); (4580 10179008 T A); (4581 10179662 A
G); (4582 10179678 A G); (4583 10179760 C T); (4584 10179839 C T); (4585 10179878 A G); (4586
10180791 A G); (4587 10181161 T C); (4588 10181445 A G); (4589 10181606 T C); (4590 10181613 A
G); (4591 10181762 G A); (4592 10181871 G A); (4593 10182458 T C); (4594 10182477 A G); (4595
10182785 C T); (4596 10183018 C T); (4597 10183032 A G); (4598 10183040 T C); (4599 10183049 A
G); (4600 10183114 T C); (4601 10183523 C A); (4602 10183573 A T); (4603 10183801 T C); (4604
10184392 T C); (4605 10184480 G A); (4606 10184482 G A); (4607 10184507 A G); (4608 10184608 A
C); (4609 10184615 G A); (4610 10184739 A G); (4611 10184771 G T); (4612 10184907 C T); (4613
10185299 T G); (4614 10185521 G A); (4615 10185632 T C); (4616 10186558 C T); (4617 10186638 A
G); (4618 10186835 C T); (4619 10187267 C T); (4620 10187389 C T); (4621 10187486 G A); (4622
10187620 G A); (4623 10189978 G A); (4624 10189991 G A); (4625 10190241 A T); (4626 10190242 T
A); (4627 10191345 C T); (4628 10191590 C T); (4629 10191591 G A); (4630 10191967 C T); (4631
10192024 A T); (4632 10192616 GA G); (4633 10192793 C A); (4634 10193482 G A); (4635 10193819 A
G); (4636 10193886 G A); (4637 10194041 C T); (4638 10194063 C A); (4639 10195420 A T); (4640
10195421 T A); (4641 10195422 A T); (4642 10195423 T A); (4643 10195424 A T); (4644 10195824 A
T); (4645 10196747 G A); (4646 10196877 C T); (4647 10197525 A C); (4648 10199690 G A); (4649
10201121 A C); (4650 10201796 A G); (4651 10202303 C T); (4652 10202347 A G); (4653 10202410 G
A); (4654 10202460 C T); (4655 10202691 G A); (4656 10203192 A G); (4657 10203197 A T); (4658
10204559 AT A); (4659 10206662 C T); (4660 10206916 A G); (4661 10207533 A T); (4662 10208612 G
C); (4663 10208756 C T); (4664 10208958 TC T); (4665 10209350 A G); (4666 10209732 G A); (4667
10210282 G A); (4668 10210311 A T); (4669 10210839 T G); (4670 10211301 C A); (4671 10211464 T
C); (4672 10211505 T C); (4673 10211679 T C); (4674 10211813 G A); (4675 10212095 A C); (4676
10212244 A C); (4677 10212292 T C); (4678 10212654 T C); (4679 10212663 T C); (4680 10212675 C
G); (4681 10212839 G A); (4682 10212902 G A); (4683 10213001 C T); (4684 10213452 A G); (4685
10213971 C T); (4686 10213991 G A); (4687 10213993 T C); (4688 10214017 A G); (4689 10214145 G
A); (4690 10214619 T C); (4691 10214828 C T); (4692 10214884 C T); (4693 10215839 GGAC G); (4694
10216119 A T); (4695 10216387 C T); (4696 10216689 A G); (4697 10216713 C G); (4698 10217630 C
CTTTCA); (4699 10217871 G A); (4700 10217913 G A); (4701 10218116 G A); (4702 10218447 A G); (4703
10218997 C T); (4704 10219191 T G); (4705 10219250 A C); (4706 10219311 G C); (4707 10219869 C
T); (4708 10220010 T C); (4709 10220021 G A); (4710 10220119 G A); (4711 10220632 T C); (4712
10220874 T C); (4713 10221022 C T); (4714 10221938 G A); (4715 10222445 A G); (4716 10222622 G
C); (4717 10222632 A G); (4718 10222932 T C); (4719 10223175 G A); (4720 10223835 G A); (4721
10223873 A G); (4722 10224088 C T); (4723 10224105 C T); (4724 10224283 G A); (4725 10224473 G
T); (4726 10224594 T C); (4727 10224799 G A); (4728 10225256 T C); (4729 10226166 A T); (4730
10226301 T G); (4731 10226317 G A); (4732 10226460 T G); (4733 10226500 A G); (4734 10226639 A
T); (4735 10226686 A G); (4736 10226872 A G); (4737 10226885 C A); (4738 10227031 A C); (4739
10228234 G C); (4740 10228458 A T); (4741 10228794 A T); (4742 10228971 C T); (4743 10229034 G
C); (4744 10229292 T A); (4745 10229661 C G); (4746 10230033 G A); (4747 10230302 G A); (4748
10231101 A G); (4749 10231246 A G); (4750 10231251 A G); (4751 10231296 T C); (4752 10231884 T
TG); (4753 10231950 C T); (4754 10232337 C T); (4755 10232592 G C); (4756 10232717 T C); (4757
10232783 A G); (4758 10232905 A T); (4759 10232996 T C); (4760 10233286 C T); (4761 10233743 C
T); (4762 10234047 T C); (4763 10234122 T G); (4764 10234154 T C); (4765 10234252 C T); (4766
10234954 T A); (4767 10235263 T C); (4768 10235385 A G); (4769 10235468 A G); (4770 10235485 T
A); (4771 10235612 C T); (4772 10235735 A G); (4773 10236264 T C); (4774 10236606 G A); (4775
10236911 T C); (4776 10236980 C A); (4777 10237209 A G); (4778 10237479 G A); (4779 10238174 C
A); (4780 10238270 T C); (4781 10238334 G A); (4782 10238343 A G); (4783 10238646 C T); (4784
10238916 A C); (4785 10238925 T A); (4786 10239038 A G); (4787 10239047 T C); (4788 10239065 T
C); (4789 10239362 C T); (4790 10240464 T C); (4791 10240492 A C); (4792 10241240 T C); (4793
10241449 A C); (4794 10241527 C T); (4795 10242449 A G); (4796 10242460 C T); (4797 10242651 A
G); (4798 10242959 C T); (4799 10243058 T C); (4800 10243170 G C); (4801 10243470 T
TTTACCCTTCGTGACGCAACATGACTGCTGG); (4802 10243702 G A); (4803 10244463 G A); (4804
10244743 CA C); (4805 10245039 T C); (4806 10245205 A C); (4807 10245451 C A); (4808 10245555 T
C); (4809 10245600 G T); (4810 10245896 G T); (4811 10245943 T C); (4812 10245981 G A); (4813
10246049 G A); (4814 10246092 G A); (4815 10246484 G A); (4816 10246577 T TA); (4817 10246764 A
G); (4818 10246792 A AATT); (4819 10247356 G A); (4820 10247617 T C); (4821 10247703 A G); (4822
10247713 G A); (4823 10247740 A G); (4824 10247745 A G); (4825 10247783 T C); (4826 10247813 A
G); (4827 10247864 T C); (4828 10247889 A G); (4829 10247956 G A); (4830 10247979 G A); (4831
10248291 G A); (4832 10248427 G A); (4833 10248446 T C); (4834 10248462 G A); (4835 10248505 T
C); (4836 10248729 A G); (4837 10248751 G A); (4838 10248755 G A); (4839 10248850 C T); (4840
10249002 G A); (4841 10249288 G A); (4842 10249845 T C); (4843 10249898 G A); (4844 10249983 A
G); (4845 10250066 A G); (4846 10250113 T C); (4847 10250137 G A); (4848 10250487 C T); (4849
10250503 C T); (4850 10250526 C T); (4851 10250864 A C); (4852 10250878 G T); (4853 10251014 C
T); (4854 10251050 A G); (4855 10251075 C T); (4856 10251125 G A); (4857 10251280 C T); (4858
10251423 C T); (4859 10251470 C G); (4860 10251650 T C); (4861 10251671 C T); (4862 10251678 T
A); (4863 10251695 A G); (4864 10251846 T C); (4865 10251984 G T); (4866 10252005 C T); (4867
10252319 G A); (4868 10252689 A T); (4869 10253120 TA T); (4870 10253315 A AT); (4871 10254004 C
T); (4872 10254134 A T); (4873 10254338 G A); (4874 10255731 TGCAGTG T); (4875 10257126 G
A); (4876 10257736 A G); (4877 10257894 A C); (4878 10258129 T C); (4879 10258178 C T); (4880
10258312 A G); (4881 10258338 G A); (4882 10258605 G A); (4883 10258710 A G); (4884 10258729 G
A); (4885 10259128 C T); (4886 10259199 G A); (4887 10259559 C T); (4888 10259578 T C); (4889
10259740 A C); (4890 10259861 G A); (4891 10259967 A G); (4892 10260044 T C); (4893 10260146 A
G); (4894 10260288 A G); (4895 10260325 A G); (4896 10260464 A C); (4897 10260936 A G); (4898
10260956 A C); (4899 10261237 A T); (4900 10261736 A T); (4901 10261775 A G); (4902 10262132 T
C); (4903 10262570 A G); (4904 10262796 T C); (4905 10262854 G A); (4906 10263140 A T); (4907
10263275 A G); (4908 10263860 G A); (4909 10264058 G A); (4910 10264061 T C); (4911 10264911 AT
A); (4912 10265151 A G); (4913 10265202 A G); (4914 10265321 G T); (4915 10265322 C T); (4916
10265345 A G); (4917 10265421 G T); (4918 10265484 G A); (4919 10265526 T C); (4920 10265781 C
G); (4921 10266444 A G); (4922 10267044 T C); (4923 10267764 A G); (4924 10268586 C A); (4925
10268696 T C); (4926 10268813 C T); (4927 10268872 G A); (4928 10269069 A G); (4929 10269233 G
A); (4930 10269303 G A); (4931 10269393 A G); (4932 10269434 A G); (4933 10269499 T G); (4934
10269500 C A); (4935 10269604 T C); (4936 10269803 T G); (4937 10269977 T C); (4938 10269998 T
C); (4939 10270115 G A); (4940 10270527 C T); (4941 10271114 A C); (4942 10272009 A G); (4943
10272577 G A); (4944 10273267 C G); (4945 10273584 T A); (4946 10273595 C T); (4947 10273918 C
T); (4948 10274272 G T); (4949 10274564 T A); (4950 10274970 A G); (4951 10275265 C T); (4952
10275554 A G); (4953 10275803 T C); (4954 10279164 A G); (4955 10279311 A G); (4956 10279561 A
C); (4957 10279573 G A); (4958 10279645 G T); (4959 10279871 T G); (4960 10279888 A G); (4961
10280435 A G); (4962 10280535 A T); (4963 10280613 C G); (4964 10281336 G A); (4965 10281338 T
A); (4966 10281910 T C); (4967 10281934 T C); (4968 10282414 A T); (4969 10282419 C T); (4970
10282502 A G); (4971 10282699 T C); (4972 10282896 C A); (4973 10283100 C G); (4974 10284403 C
T); (4975 10284502 C T); (4976 10285123 T C); (4977 10285184 G T); (4978 10285513 A T); (4979
10285861 C T); (4980 10285863 G C); (4981 10286276 A AG); (4982 10286576 T C); (4983 10287194 C
G); (4984 10287211 C T); (4985 10287282 A C); (4986 10287408 A C); (4987 10287586 A T); (4988
10287794 T A); (4989 10287862 C T); (4990 10289640 T C); (4991 10289809 T C); (4992 10289924 G
T); (4993 10290427 C T); (4994 10290622 A C); (4995 10290623 T G); (4996 10290625 A T); (4997
10290626 T C); (4998 10290627 C A); (4999 10290628 A T); (5000 10290797 C G); (5001 10290951 T
A); (5002 10291473 T C); (5003 10291656 C T); (5004 10291762 C G); (5005 10291979 C T); (5006
10292046 G A); (5007 10292071 C G); (5008 10292347 T C); (5009 10292441 C T); (5010 10292542 A
G); (5011 10292586 A G); (5012 10292842 T C); (5013 10293076 G A); (5014 10293117 T C); (5015
10293211 T C); (5016 10293951 T C); (5017 10294218 A G); (5018 10294277 C T); (5019 10294288 C
T); (5020 10295046 G A); (5021 10295117 A AG); (5022 10295234 T C); (5023 10295456 C T); (5024
10295495 C G); (5025 10295780 G A); (5026 10295806 A G); (5027 10295917 A G); (5028 10296086 T
C); (5029 10296234 G A); (5030 10296304 G T); (5031 10297780 A G); (5032 10297999 G A); (5033
10298351 C G); (5034 10298406 A AG); (5035 10298421 G A); (5036 10298423 T C); (5037 10298531 G
A); (5038 10298775 G T); (5039 10299022 T C); (5040 10299031 A G); (5041 10299154 A G); (5042
10299461 T G); (5043 10299633 G A); (5044 10299799 G A); (5045 10299892 C T); (5046 10300015 T
C); (5047 10300384 T C); (5048 10300568 C T); (5049 10300576 G A); (5050 10300693 A G); (5051
10300853 G A); (5052 10301201 A G); (5053 10301295 T C); (5054 10301761 G A); (5055 10301786 G
A); (5056 10301800 C T); (5057 10301944 C T); (5058 10302115 T C); (5059 10302427 G A); (5060
10302794 C T); (5061 10302992 G C); (5062 10303704 T C); (5063 10303761 A C); (5064 10303830 G
A); (5065 10303921 A T); (5066 10304143 G A); (5067 10305092 A G); (5068 10305380 A T); (5069
10305628 G T); (5070 10306541 T A); (5071 10307463 G A); (5072 10307804 T C); (5073 10307915 G
A); (5074 10308027 C G); (5075 10308227 A T); (5076 10308253 T C); (5077 10309238 T TA); (5078
10309583 GA G); (5079 10309672 T A); (5080 10310245 G T); (5081 10310610 A G); (5082 10310997 C
G); (5083 10311350 AT A); (5084 10311493 A G); (5085 10311684 G A); (5086 10311861 C T); (5087
10312227 A G); (5088 10312231 G C); (5089 10312484 C G); (5090 10312485 A G); (5091 10312488 T
C); (5092 10312654 T A); (5093 10312670 A G); (5094 10312872 C T); (5095 10312887 T C); (5096
10312973 C T); (5097 10313264 T C); (5098 10313534 C T); (5099 10313639 T C); (5100 10314042 C
T); (5101 10314079 C T); (5102 10314085 T C); (5103 10314147 C T); (5104 10314165 C T); (5105
10314208 G A); (5106 10314311 C G); (5107 10314393 C T); (5108 10315025 C T); (5109 10315028 G
A); (5110 10315079 C T); (5111 10315378 T C); (5112 10315877 A G); (5113 10315978 G A); (5114
10316464 C T); (5115 10316487 G A); (5116 10316656 T A); (5117 10316947 T C); (5118 10317083 G
A); (5119 10317338 C T); (5120 10317351 C T); (5121 10317352 C T); (5122 10317566 T C); (5123
10317642 C T); (5124 10317650 T C); (5125 10317669 C A); (5126 10317707 C T); (5127 10318017 C
T); (5128 10318262 A G); (5129 10318478 C T); (5130 10318609 T C); (5131 10318879 T A); (5132
10318926 G T); (5133 10319167 G A); (5134 10319254 ATGCTG A); (5135 10319502 T C); (5136
10319555 A G); (5137 10319594 C G); (5138 10319714 G A); (5139 10319944 C T); (5140 10319993 C
A); (5141 10320527 G C); (5142 10320713 C T); (5143 10320733 GA G); (5144 10320742 G A); (5145
10320753 C T); (5146 10321858 T G); (5147 10321963 C CTG); (5148 10322852 G A); (5149 10322993 T
C); (5150 10323123 G A); (5151 10323338 GT G); (5152 10324953 G C); (5153 10325415 T A); (5154
10326140 G A); (5155 10326182 C T); (5156 10326569 T C); (5157 10326745 T C); (5158 10327149 G
T); (5159 10327309 A G); (5160 10327535 G A); (5161 10328589 G A); (5162 10328623 T G); (5163
10328697 T G); (5164 10328995 C T); (5165 10329163 A G); (5166 10329198 G A); (5167 10330889 C
T); (5168 10331027 G A); (5169 10331056 A C); (5170 10331058 C T); (5171 10331201 T C); (5172
10331294 A C); (5173 10332122 C T); (5174 10332763 C A); (5175 10333404 G T); (5176 10333880 G
T); (5177 10334190 T C); (5178 10334417 C T); (5179 10334697 A G); (5180 10335245 C A); (5181
10335343 G A); (5182 10335496 C T); (5183 10335866 A G); (5184 10336011 G A); (5185 10336113 C
T); (5186 10336128 A G); (5187 10336227 C G); (5188 10336240 T TTTCACAAGAAAACCGA); (5189
10336317 T A); (5190 10336386 C T); (5191 10336410 G A); (5192 10336880 C T); (5193 10337396 C
T); (5194 10337445 A G); (5195 10337479 A G); (5196 10337510 C T); (5197 10338579 A G); (5198
10338994 T C); (5199 10339284 G A); (5200 10342374 G A); (5201 10342603 G A); (5202 10342691 TA
T); (5203 10343130 A G); (5204 10343503 TA T); (5205 10343888 A T); (5206 10343910 C T); (5207
10344109 T C); (5208 10344160 C A); (5209 10344458 C A); (5210 10344595 T G); (5211 10344612 A
G); (5212 10346041 A G); (5213 10346050 T C); (5214 10347073 C T); (5215 10347881 G T); (5216
10348101 A G); (5217 10348833 GTTAAAT G); (5218 10349070 T C); (5219 10349467 G A); (5220
10350611 C T); (5221 10351916 C T); (5222 10352123 C T); (5223 10352168 C A); (5224 10353985 G
A); (5225 10354202 C T); (5226 10354702 T G); (5227 10355093 A AT); (5228 10356845 A AT); (5229
10357218 C T); (5230 10358135 T TA); (5231 10358485 TA T); (5232 10359153 GA G); (5233 10360041 C
A); (5234 10360149 G A); (5235 10361243 T C); (5236 10361391 G A); (5237 10361625 C T); (5238
10362127 T C); (5239 10362167 A G); (5240 10363298 C A); (5241 10363626 T C); (5242 10364141 A
T); (5243 10364197 C A); (5244 10364311 T A); (5245 10364312 T A); (5246 10364322 T C); (5247
10364350 A AT); (5248 10364668 G A); (5249 10364676 G A); (5250 10364742 T A); (5251 10364974 C
T); (5252 10365115 C T); (5253 10365128 C T); (5254 10365131 G A); (5255 10365325 C T); (5256
10365379 G A); (5257 10365424 GA G); (5258 10365666 GA G); (5259 10365840 A C); (5260 10366004 G
GT); (5261 10366053 T C); (5262 10367812 A C); (5263 10368032 G A); (5264 10368392 G A); (5265
10368506 G A); (5266 10368583 A AT); (5267 10369072 A G); (5268 10369098 A G); (5269 10369457 G
T); (5270 10370978 C T); (5271 10371272 G C); (5272 10371311 T G); (5273 10371485 C T); (5274
10371524 G A); (5275 10371781 A G); (5276 10372050 A G); (5277 10372421 T G); (5278 10372461 A
AGGAGTTATTTGAGGACAC); (5279 10372919 C T); (5280 10373031 T G); (5281 10373032 C T); (5282
10373220 C T); (5283 10374192 GA G); (5284 10374405 T C); (5285 10374576 T C); (5286 10374706 T
A); (5287 10374788 T A); (5288 10374888 A G); (5289 10375220 G A); (5290 10375330 T C); (5291
10375402 G A); (5292 10375417 A C); (5293 10375468 A G); (5294 10375560 C T); (5295 10375582 A
G); (5296 10375954 A G); (5297 10376188 G A); (5298 10376431 T C); (5299 10376754 T C); (5300
10377099 G A); (5301 10377595 G A); (5302 10377616 T C); (5303 10377840 G A); (5304 10377928 G
A); (5305 10378295 T C); (5306 10378583 C A); (5307 10378803 G A); (5308 10379161 T C); (5309
10379280 A G); (5310 10379892 A G); (5311 10380020 G T); (5312 10380698 T A); (5313 10380788 T
C); (5314 10380839 AAG A); (5315 10380994 A C); (5316 10381347 G A); (5317 10381872 C T); (5318
10382196 T C); (5319 10382493 G A); (5320 10382836 G A); (5321 10383453 C T); (5322 10383977 C
G); (5323 10384115 A C); (5324 10384263 A G); (5325 10384532 T C); (5326 10384662 G A); (5327
10385399 C A); (5328 10385629 C G); (5329 10385843 G C); (5330 10387102 T G); (5331 10387217 C
T); (5332 10387263 C T); (5333 10387294 G T); (5334 10388244 T G); (5335 10388520 G A); (5336
10388676 C T); (5337 10389126 G T); (5338 10389634 A C); (5339 10389697 G A); (5340 10390032 C
T); (5341 10390311 G T); (5342 10390667 G C); (5343 10390754 C A); (5344 10390928 T A); (5345
10391173 A G); (5346 10391280 C A); (5347 10391624 AAC A); (5348 10391631 T A); (5349 10391638 T
C); (5350 10391848 C A); (5351 10391871 A G); (5352 10392671 C A); (5353 10393129 T C); (5354
10393413 G A); (5355 10393487 G C); (5356 10393607 A AC); (5357 10394180 G A); (5358 10394232 T
C); (5359 10394345 A G); (5360 10394380 T C); (5361 10394437 A G); (5362 10394675 G A); (5363
10394960 C T); (5364 10394968 A G); (5365 10395267 G A); (5366 10395290 AT A); (5367 10395294 A
C); (5368 10395310 A G); (5369 10395762 A T); (5370 10395940 G A); (5371 10396309 T C); (5372
10396645 T C); (5373 10397032 T C); (5374 10397342 G C); (5375 10397482 A C); (5376 10397512 G
A); (5377 10397625 G A); (5378 10397665 G T); (5379 10397690 A G); (5380 10397748 C G); (5381
10397897 G A); (5382 10397999 C T); (5383 10398415 T C); (5384 10398643 G C); (5385 10398791 C
T); (5386 10398816 A G); (5387 10398936 C T); (5388 10399828 C T); (5389 10400052 G A); (5390
10400677 T C); (5391 10400701 G T); (5392 10400987 A G); (5393 10401097 T TG); (5394 10401629 G
T); (5395 10401826 G A); (5396 10401897 G A); (5397 10402436 C T); (5398 10403500 T G); (5399
10403569 T C); (5400 10403763 C G); (5401 10403788 C T); (5402 10403928 C T); (5403 10404071 G
A); (5404 10404295 G T); (5405 10404469 A G); (5406 10404593 G A); (5407 10404616 A C); (5408
10404660 G A); (5409 10404898 C A); (5410 10405778 C T); (5411 10406102 T C); (5412 10406205 C
T); (5413 10406729 T A); (5414 10407293 G C); (5415 10407504 T A); (5416 10409726 C T); (5417
10410655 T G); (5418 10410790 C T); (5419 10410888 T G); (5420 10411569 C A); (5421 10411608 C
T); (5422 10411759 G A); (5423 10411761 G A); (5424 10412210 G T); (5425 10412326 G A); (5426
10412638 A G); (5427 10412704 G A); (5428 10413014 C T); (5429 10413236 G A); (5430 10413926 C
T); (5431 10414928 C T); (5432 10415313 T C); (5433 10415499 A C); (5434 10415651 T C); (5435
10415707 G A); (5436 10415740 C T); (5437 10416252 T C); (5438 10416343 C T); (5439 10416473 G
A); (5440 10416526 A T); (5441 10416556 A G); (5442 10416695 T TAAATTTC); (5443 10416768 G
A); (5444 10417459 T C); (5445 10418045 C T); (5446 10418120 G A); (5447 10418514 G A); (5448
10418716 T G); (5449 10418730 G A); (5450 10419687 A C); (5451 10420010 G A); (5452 10420121 T
G); (5453 10421182 T G); (5454 10421188 C T); (5455 10421219 G A); (5456 10421229 T A); (5457
10421289 C T); (5458 10421292 G T); (5459 10421328 T C); (5460 10421390 G A); (5461 10421404 C
T); (5462 10421409 G A); (5463 10421424 T C); (5464 10421568 C T); (5465 10421599 C T); (5466
10421860 G T); (5467 10422004 G A); (5468 10422521 C T); (5469 10422613 C T); (5470 10422902 A
G); (5471 10422994 G A); (5472 10423405 T G); (5473 10423658 G T); (5474 10423687 G A); (5475
10423857 T C); (5476 10424291 T C); (5477 10424868 C G); (5478 10424996 C T); (5479 10425223 A
G); (5480 10425270 C T); (5481 10425701 T A); (5482 10425742 AAAT A); (5483 10425830 G T); (5484
10426081 G A); (5485 10426095 GA G); (5486 10426376 C A); (5487 10426730 C T); (5488 10426958 C
A); (5489 10426989 G A); (5490 10428005 C T); (5491 10428404 A G); (5492 10428458 C T); (5493
10429438 T A); (5494 10429565 A G); (5495 10429648 C T); (5496 10429668 C T); (5497 10429821 T
A); (5498 10436117 C A); (5499 10436122 C G); (5500 10436647 A C); (5501 10437057 T G); (5502
10437179 T C); (5503 10437341 C A); (5504 10437489 C G); (5505 10438180 T C); (5506 10438253 A
AGCATGAATTGCATGAT); (5507 10438445 A G); (5508 10438462 T C); (5509 10439146 C G); (5510
10439167 T C); (5511 10439272 G T); (5512 10439437 C T); (5513 10439780 A G); (5514 10440013 C
T); (5515 10440369 A G); (5516 10440635 T G); (5517 10440736 A G); (5518 10441089 A T); (5519
10441091 G C); (5520 10441092 T G); (5521 10441384 A G); (5522 10441446 C T); (5523 10441575 G
T); (5524 10441693 T A); (5525 10441726 G A); (5526 10441836 C T); (5527 10441900 G A); (5528
10441926 C T); (5529 10442094 T C); (5530 10442095 T C); (5531 10442099 C T); (5532 10442100 G
A); (5533 10442142 A G); (5534 10442154 C T); (5535 10442163 T C); (5536 10442208 A G); (5537
10442390 C A); (5538 10442424 A AATAGAGGTTCC); (5539 10442567 C T); (5540 10442583 C T); (5541
10442598 G A); (5542 10443259 G A); (5543 10443403 A C); (5544 10443576 T C); (5545 10443741 T
C); (5546 10443927 G A); (5547 10444271 C T); (5548 10444380 A G); (5549 10444610 G A); (5550
10444935 T G); (5551 10446480 A C); (5552 10446481 G A); (5553 10446709 G A); (5554 10446722 G
A); (5555 10447036 G A); (5556 10447064 C T); (5557 10447327 T C); (5558 10447800 A G); (5559
10447872 T C); (5560 10447954 G T); (5561 10447963 G T); (5562 10448079 C T); (5563 10448209 G
A); (5564 10448277 G A); (5565 10448314 T C); (5566 10448534 G A); (5567 10448697 C T); (5568
10448710 C A); (5569 10448842 G A); (5570 10448854 G A); (5571 10449022 C T); (5572 10449105 A
G); (5573 10449114 T C); (5574 10449154 G A); (5575 10449171 G A); (5576 10449318 T C); (5577
10449491 G A); (5578 10449587 C CA); (5579 10449777 G A); (5580 10449993 T C); (5581 10450005 T
C); (5582 10450781 C T); (5583 10450805 C G); (5584 10450957 A G); (5585 10451093 G A); (5586
10451103 A G); (5587 10451230 A C); (5588 10451247 G A); (5589 10451332 A T); (5590 10451605 A
G); (5591 10451987 A C); (5592 10452166 T A); (5593 10452273 C A); (5594 10452308 G A); (5595
10452392 C T); (5596 10452454 C A); (5597 10454954 A G); (5598 10455720 G A); (5599 10456109 G
T); (5600 10456190 A G); (5601 10457553 C A); (5602 10457559 T C); (5603 10457655 A G); (5604
10458084 A C); (5605 10458451 G T); (5606 10458452 C T); (5607 10458783 T C); (5608 10458843 C
T); (5609 10458936 T C); (5610 10458948 A G); (5611 10458949 C T); (5612 10459140 T C); (5613
10459174 C A); (5614 10463733 A G); (5615 10463781 C T); (5616 10463819 T C); (5617 10465197 A
G); (5618 10465283 C T); (5619 10465842 G A); (5620 10465845 C T); (5621 10466063 C T); (5622
10466270 C T); (5623 10466557 G GGAAAAAGAAAATGAATGT); (5624 10466661 C T); (5625 10466865 G
GCAGGTCCTCTTAATTCTTTTT); (5626 10467040 C T); (5627 10467156 T C); (5628 10467260 A G); (5629
10467348 C T); (5630 10467828 T C); (5631 10467896 A G); (5632 10467900 C T); (5633 10468301 C
T); (5634 10468387 T G); (5635 10468435 C CTT); (5636 10468895 TTGTAA T); (5637 10468983 A
G); (5638 10469581 A C); (5639 10470037 T C); (5640 10470070 T C); (5641 10470473 A G); (5642
10470562 C A); (5643 10471133 T A); (5644 10471158 G A); (5645 10472286 G T); (5646 10472475 A
T); (5647 10473111 G T); (5648 10473114 T C); (5649 10473852 T C); (5650 10474643 T G); (5651
10475559 A T); (5652 10475711 T C); (5653 10475724 C T); (5654 10475878 C T); (5655 10475982 G
A); (5656 10476522 G A); (5657 10476624 T G); (5658 10476796 G A); (5659 10476872 T C); (5660
10477120 G A); (5661 10477168 T C); (5662 10477382 G A); (5663 10477468 G A); (5664 10477682 G
A); (5665 10477729 A T); (5666 10477881 G A); (5667 10477974 A G); (5668 10477991 G A); (5669
10478186 C A); (5670 10478230 A G); (5671 10478390 A G); (5672 10478470 G T); (5673 10478514 G
A); (5674 10478524 G A); (5675 10478526 G A); (5676 10478812 C T); (5677 10479055 C T); (5678
10479111 G A); (5679 10479285 C T); (5680 10479404 G A); (5681 10479453 C T); (5682 10479666 C
T); (5683 10479690 T C); (5684 10479696 G A); (5685 10479697 A G); (5686 10479796 C T); (5687
10480301 T C); (5688 10480373 A AT); (5689 10481187 A G); (5690 10481212 C T); (5691 10481254 G
A); (5692 10481366 A G); (5693 10481460 C T); (5694 10481902 C A); (5695 10481987 C T); (5696
10482118 T G); (5697 10482252 C T); (5698 10482259 G A); (5699 10482665 C T); (5700 10482789 A
G); (5701 10482978 G T); (5702 10483376 G A); (5703 10483441 T G); (5704 10483558 G A); (5705
10483765 G A); (5706 10483807 G T); (5707 10484074 G A); (5708 10484153 G A); (5709 10484270 C
G); (5710 10484633 C T); (5711 10484953 G A); (5712 10486189 G A); (5713 10486490 A G); (5714
10486817 A G); (5715 10487176 A C); (5716 10487638 CAT C); (5717 10488243 A G); (5718 10488249
GA G); (5719 10488359 G A); (5720 10488829 A T); (5721 10488876 G A); (5722 10489061 A G); (5723
10489084 G A); (5724 10489212 G A); (5725 10489215 G A); (5726 10489459 C T); (5727 10489920 C
G); (5728 10490478 G T); (5729 10490525 ATGATACTCTGTT A); (5730 10490779 A C); (5731 10491093 G
A); (5732 10491289 T C); (5733 10491525 T C); (5734 10491875 A G); (5735 10491986 G A); (5736
10492176 C T); (5737 10492269 C T); (5738 10492433 G A); (5739 10492690 T C); (5740 10493029 G
A); (5741 10493398 C T); (5742 10493656 C T); (5743 10493821 G A); (5744 10493872 A G); (5745
10494296 C T); (5746 10494546 C T); (5747 10495092 T C); (5748 10495358 A T); (5749 10495359 G
A); (5750 10495447 C A); (5751 10495456 C T); (5752 10495903 G T); (5753 10495950 G T); (5754
10496087 G A); (5755 10496268 G A); (5756 10496287 A G); (5757 10496629 A G); (5758 10496862 G
A); (5759 10497068 G A); (5760 10497103 G GATTGAAAACTGATTTTGAT); (5761 10497237 A G); (5762
10497281 C T); (5763 10497455 A T); (5764 10497796 C T); (5765 10498123 C T); (5766 10498373 C
A); (5767 10498456 ATG A); (5768 10499247 T C); (5769 10499270 A C); (5770 10499293 C T); (5771
10499673 T C); (5772 10499884 A G); (5773 10499885 G A); (5774 10500050 C T); (5775 10500713 G
C); (5776 10500745 C T); (5777 10501040 G C); (5778 10501269 G A); (5779 10501547 C T); (5780
10501627 C T); (5781 10501872 T A); (5782 10502028 C T); (5783 10502137 T C); (5784 10502481 C
T); (5785 10502512 A G); (5786 10502513 C A); (5787 10502625 T C); (5788 10502707 C A); (5789
10502771 G A); (5790 10503047 G A); (5791 10503620 G A); (5792 10504017 T A); (5793 10504200 G
A); (5794 10504222 G A); (5795 10504231 G A); (5796 10504293 A C); (5797 10504301 T A); (5798
10504426 C A); (5799 10504442 T C); (5800 10504749 T C); (5801 10504820 T C); (5802 10504937 T
A); (5803 10504992 A G); (5804 10505004 A C); (5805 10505042 G A); (5806 10505201 T C); (5807
10506427 G A); (5808 10506699 G A); (5809 10507244 A G); (5810 10507780 C T); (5811 10507791 G
T); (5812 10507819 C G); (5813 10507844 T G); (5814 10508008 G A); (5815 10508067 G C); (5816
10508255 C A); (5817 10508275 A G); (5818 10508366 G A); (5819 10508395 T C); (5820 10508457 C
G); (5821 10508680 T C); (5822 10509643 A G); (5823 10510178 A C); (5824 10510846 G A); (5825
10510969 A C); (5826 10511155 G A); (5827 10511468 A G); (5828 10511637 T C); (5829 10511745 C
T); (5830 10511886 C A); (5831 10512256 C T); (5832 10512521 A G); (5833 10512583 TC T); (5834
10513134 G A); (5835 10513240 G A); (5836 10513642 A T); (5837 10513646 G A); (5838 10513999 C
T); (5839 10514198 C T); (5840 10514341 G A); (5841 10514790 A G); (5842 10515074 T G); (5843
10515284 C T); (5844 10515287 C T); (5845 10515426 C T); (5846 10515692 C T); (5847 10516070 T
C); (5848 10516281 C T); (5849 10516368 C CT); (5850 10516592 G A); (5851 10517560 C A); (5852
10517884 C A); (5853 10518001 G C); (5854 10518011 C T); (5855 10518022 G A); (5856 10518270 G
T); (5857 10518271 T C); (5858 10518297 A G); (5859 10518418 A G); (5860 10518489 C A); (5861
10518627 T C); (5862 10518723 A T); (5863 10518934 G A); (5864 10519018 C A); (5865 10519019 A
C); (5866 10519022 C A); (5867 10519023 G C); (5868 10519027 A G); (5869 10519028 T A); (5870
10519029 A T); (5871 10519231 C T); (5872 10519547 A T); (5873 10519600 A G); (5874 10519643 A
G); (5875 10519852 T G); (5876 10520090 T C); (5877 10520101 T C); (5878 10520151 G
GAAATACACTA); (5879 10521446 C A); (5880 10522081 T C); (5881 10522637 A T); (5882 10523414 A
C); (5883 10523561 C A); (5884 10523610 C T); (5885 10523707 C T); (5886 10523747 C T); (5887
10523863 C G); (5888 10523930 C G); (5889 10524909 C A); (5890 10525204 G A); (5891 10525361 A
G); (5892 10525602 G T); (5893 10525644 A G); (5894 10525867 A C); (5895 10525939 T G); (5896
10525985 G A); (5897 10526041 G T); (5898 10526057 A G); (5899 10526163 G A); (5900 10526497 G
A); (5901 10526507 A G); (5902 10526655 G T); (5903 10526776 A G); (5904 10527260 C T); (5905
10527280 A G); (5906 10527378 A T); (5907 10527788 T C); (5908 10528202 C T); (5909 10528440 C
CAA); (5910 10528521 A C); (5911 10528839 C T); (5912 10529130 C T); (5913 10529317 T C); (5914
10529364 C T); (5915 10529598 A G); (5916 10529626 A G); (5917 10529761 C T); (5918 10529971 T
C); (5919 10530373 A T); (5920 10530383 C A); (5921 10530605 T C); (5922 10530610 G C); (5923
10531315 AT A); (5924 10531424 T C); (5925 10531538 C T); (5926 10531874 T C); (5927 10532056 C
T); (5928 10532205 G A); (5929 10532446 C T); (5930 10532696 A G); (5931 10533092 A G); (5932
10533153 T C); (5933 10533220 T A); (5934 10533256 A G); (5935 10533384 G A); (5936 10533523 A
G); (5937 10533738 T G); (5938 10533772 G A); (5939 10534105 A G); (5940 10534275 G A); (5941
10534513 C T); (5942 10534568 T C); (5943 10535513 C G); (5944 10535678 T C); (5945 10535922 A
G); (5946 10536595 C T); (5947 10536638 T A); (5948 10536639 G A); (5949 10536755 A G); (5950
10537062 T A); (5951 10537450 G A); (5952 10537456 C T); (5953 10537503 T G); (5954 10537878 G
A); (5955 10537913 G A); (5956 10538294 GA G); (5957 10538419 G A); (5958 10538693 C T); (5959
10538869 T C); (5960 10538937 G C); (5961 10539045 G A); (5962 10539234 C G); (5963 10539329 A
T); (5964 10539442 A G); (5965 10539450 C A); (5966 10539519 A G); (5967 10539806 T C); (5968
10539846 A G); (5969 10540128 T C); (5970 10540129 C A); (5971 10540216 A G); (5972 10540264 T
C); (5973 10540288 G A); (5974 10540300 C T); (5975 10540599 A G); (5976 10540723 T A); (5977
10540791 G A); (5978 10541577 GATTCTCTTAA G); (5979 10542374 T A); (5980 10543174 CTTTATTTA
C); (5981 10543524 T C); (5982 10543650 A G); (5983 10543884 C G); (5984 10548286 C T); (5985
10548332 C T); (5986 10548845 A T); (5987 10548863 A G); (5988 10548956 G A); (5989 10548957 C
T); (5990 10549299 T C); (5991 10549302 G A); (5992 10549500 T C); (5993 10549505 C T); (5994
10549509 T C); (5995 10549510 A T); (5996 10549608 C A); (5997 10549609 T C); (5998 10549736 C
A); (5999 10549745 T C); (6000 10549749 C T); (6001 10550231 G T); (6002 10550261 G A); (6003
10550267 G A); (6004 10550268 T A); (6005 10550270 C T); (6006 10550301 C T); (6007 10550306 C
T); (6008 10550308 A G); (6009 10550311 T C); (6010 10550315 A T); (6011 10550370 G A); (6012
10550404 A G); (6013 10550406 C T); (6014 10550426 A G); (6015 10550427 C T); (6016 10550531 C
T); (6017 10550766 T C); (6018 10550777 T C); (6019 10550783 A G); (6020 10550794 T C); (6021
10550797 G A); (6022 10550819 T C); (6023 10550851 CT C); (6024 10550881 A G); (6025 10551476 C
T); (6026 10551548 G A); (6027 10551590 G A); (6028 10551596 A G); (6029 10554594 C T); (6030
10560403 T G); (6031 10560462 T A); (6032 10560883 A G); (6033 10561098 A C); (6034 10561256 A
G); (6035 10562775 T C); (6036 10562851 G A); (6037 10562852 T C); (6038 10562917 A G); (6039
10563390 ATATTT A); (6040 10563625 A T); (6041 10564636 A T); (6042 10565407 T A); (6043 10565497
G A); (6044 10565508 G A); (6045 10565757 T C); (6046 10566534 TGGAAGAAGAACAAGCCTTATATA
T); (6047 10566758 C T); (6048 10568477 T G); (6049 10569240 A C); (6050 10569317 A AT); (6051
10570251 A T); (6052 10570278 A G); (6053 10570501 T C); (6054 10571704 A C); (6055 10571983 A
C); (6056 10572784 C A); (6057 10573716 T A); (6058 10573953 C T); (6059 10574009 T C); (6060
10574016 G A); (6061 10574059 ATT A); (6062 10574236 C T); (6063 10574247 G A); (6064 10574541 G
T); (6065 10574657 G A); (6066 10574677 C G); (6067 10574956 T C); (6068 10575782 A AT); (6069
10576768 C T); (6070 10576888 A T); (6071 10577093 T TGA); (6072 10577193 C T); (6073 10577275 G
T); (6074 10577417 C T); (6075 10577624 C T); (6076 10578062 C T); (6077 10578506 G A); (6078
10578872 G T); (6079 10578987 G T); (6080 10579114 A G); (6081 10579236 G A); (6082 10579331 G
T); (6083 10579425 G A); (6084 10579440 C A); (6085 10580226 G A); (6086 10580297 C T); (6087
10580752 A T); (6088 10580861 G A); (6089 10580918 A G); (6090 10581328 G A); (6091 10581414 C
T); (6092 10581490 G A); (6093 10581736 A G); (6094 10582071 G A); (6095 10582512 C T); (6096
10582517 G C); (6097 10582958 C G); (6098 10583112 TAA T); (6099 10583116 A G); (6100 10583473 A
C); (6101 10584044 C T); (6102 10584137 G A); (6103 10584154 ATCAATTTT A); (6104 10584306 G
A); (6105 10584475 C T); (6106 10585096 C T); (6107 10585267 C A); (6108 10585756 T C); (6109
10585935 A G); (6110 10585958 C T); (6111 10585991 C T); (6112 10586030 A G); (6113 10586468 T
C); (6114 10586732 C A); (6115 10586828 T C); (6116 10587236 GA G); (6117 10588426 T A); (6118
10588442 AT A); (6119 10588541 G GAA); (6120 10588918 GA G); (6121 10589460 G C); (6122
10589477 A AT); (6123 10589527 A ACCCTTCTTTAATTTCCATG); (6124 10590143 AAGGAT A); (6125
10590885 T TA); (6126 10591073 T C); (6127 10591124 G A); (6128 10591138 G A); (6129 10591379 T
G); (6130 10591439 T C); (6131 10591449 C T); (6132 10591668 C T); (6133 10591680 G A); (6134
10591899 A AAT); (6135 10592130 A G); (6136 10592411 G A); (6137 10592719 T C); (6138 10593392 T
C); (6139 10593402 G A); (6140 10593470 C A); (6141 10593541 A G); (6142 10593698 TG T); (6143
10593779 T C); (6144 10594094 T C); (6145 10594120 C T); (6146 10594186 C T); (6147 10594277 G
A); (6148 10594328 G A); (6149 10594586 A G); (6150 10594813 A G); (6151 10594926 T C); (6152
10594938 G A); (6153 10595208 C T); (6154 10595438 G A); (6155 10595613 G A); (6156 10595959 G
A); (6157 10596856 T G); (6158 10597000 G A); (6159 10597198 A G); (6160 10597817 C T); (6161
10597873 C T); (6162 10597919 T C); (6163 10598033 T A); (6164 10598144 T C); (6165 10598178 A
G); (6166 10598204 A G); (6167 10598417 G C); (6168 10598442 G A); (6169 10598577 C A); (6170
10599155 A G); (6171 10599273 T C); (6172 10599394 T C); (6173 10599678 C T); (6174 10599680 T
C); (6175 10599819 C T); (6176 10599919 A C); (6177 10600000 C A); (6178 10600149 C T); (6179
10600575 C A); (6180 10600625 T C); (6181 10600705 T C); (6182 10600865 C T); (6183 10600993 A
G); (6184 10602197 A G); (6185 10602462 C G); (6186 10602500 A C); (6187 10602559 T C); (6188
10602635 G A); (6189 10603149 A G); (6190 10603228 C CAG); (6191 10603463 C G); (6192 10603936 A
G); (6193 10604359 G A); (6194 10604673 T C); (6195 10605623 T C); (6196 10605810 G T); (6197
10606139 C T); (6198 10606305 T G); (6199 10606771 A AT); (6200 10607721 T C); (6201 10608145 A
T); (6202 10608482 G GC); (6203 10609909 G A); (6204 10610607 C T); (6205 10610822 A G); (6206
10610979 C T); (6207 10611370 C A); (6208 10611450 G A); (6209 10611767 A G); (6210 10611915 C
A); (6211 10612340 G C); (6212 10612518 T C); (6213 10613759 A G); (6214 10613883 T C); (6215
10614154 T C); (6216 10614410 GA G); (6217 10614511 T C); (6218 10614830 T A); (6219 10616025 C
T); (6220 10616068 G T); (6221 10616121 T A); (6222 10616220 A C); (6223 10616294 G A); (6224
10616431 C A); (6225 10616760 G A); (6226 10616941 C T); (6227 10617271 A G); (6228 10617677 G
GA); (6229 10617879 T A); (6230 10617945 A G); (6231 10619714 A C); (6232 10622675 A AT); (6233
10623091 A C); (6234 10624032 A G); (6235 10624069 T C); (6236 10624083 G A); (6237 10624147 T
C); (6238 10624212 C T); (6239 10624229 G A); (6240 10624295 G T); (6241 10624492 G A); (6242
10624966 T G); (6243 10625260 C T); (6244 10625280 C T); (6245 10625297 A T); (6246 10625405 G
A); (6247 10625434 C T); (6248 10625499 C T); (6249 10625625 A G); (6250 10626244 T C); (6251
10626413 A T); (6252 10626487 C G); (6253 10626670 G A); (6254 10626760 C T); (6255 10626788 G
A); (6256 10627025 A T); (6257 10627096 G A); (6258 10627380 G A); (6259 10628018 T C); (6260
10628459 T C); (6261 10628525 A C); (6262 10628882 C A); (6263 10628952 G A); (6264 10629431 A
G); (6265 10629910 G A); (6266 10630625 A G); (6267 10630994 T C); (6268 10631104 A G); (6269
10631190 G A); (6270 10632005 C T); (6271 10632316 C A); (6272 10632633 T C); (6273 10633758 C
T); (6274 10633898 C T); (6275 10634464 A C); (6276 10634869 C T); (6277 10634908 G A); (6278
10635039 AG A); (6279 10635460 T C); (6280 10636158 A G); (6281 10636926 C G); (6282 10636948 C
T); (6283 10637095 T C); (6284 10637574 CAG C); (6285 10637598 G A); (6286 10637794 A G); (6287
10637899 G A); (6288 10638053 C T); (6289 10638384 G A); (6290 10638704 A G); (6291 10639076 T
C); (6292 10639261 G A); (6293 10639342 G T); (6294 10640014 G A); (6295 10641062 A G); (6296
10641492 T G); (6297 10641540 A T); (6298 10641541 A T); (6299 10641724 G A); (6300 10641954 T
G); (6301 10642694 T C); (6302 10642765 C T); (6303 10643615 C G); (6304 10643658 G A); (6305
10643681 T G); (6306 10643712 G A); (6307 10643744 G A); (6308 10644057 C T); (6309 10644078 A
G); (6310 10644741 C T); (6311 10644819 G A); (6312 10644937 G A); (6313 10645008 T C); (6314
10645019 C T); (6315 10645215 G A); (6316 10645391 A G); (6317 10645870 A G); (6318 10645930 G
A); (6319 10646573 C T); (6320 10646666 G A); (6321 10647700 G A); (6322 10647909 C T); (6323
10649117 C T); (6324 10649409 G C); (6325 10650222 T C); (6326 10650574 T C); (6327 10651526 A
G); (6328 10651727 A G); (6329 10652399 C A); (6330 10652424 G A); (6331 10652850 G C); (6332
10652980 C T); (6333 10653949 G A); (6334 10653959 G A); (6335 10653990 T C); (6336 10654615 C
T); (6337 10655167 C T); (6338 10655247 C G); (6339 10655683 T C); (6340 10655958 T C); (6341
10656452 C T); (6342 10656901 C A); (6343 10657031 C T); (6344 10657032 A G); (6345 10657166 C
T); (6346 10657402 T C); (6347 10657508 C T); (6348 10657572 C T); (6349 10657669 C T); (6350
10657763 A T); (6351 10657798 C T); (6352 10657810 C A); (6353 10657894 C T); (6354 10657971 G
A); (6355 10658270 C A); (6356 10658327 C T); (6357 10658439 C G); (6358 10658679 G A); (6359
10658985 G A); (6360 10659284 C T); (6361 10659501 C T); (6362 10659509 C T); (6363 10660093 G
A); (6364 10660395 T C); (6365 10660646 G A); (6366 10660765 C T); (6367 10661036 G A); (6368
10661228 G A); (6369 10661526 C T); (6370 10661825 A C); (6371 10661997 C T); (6372 10662623 A
G); (6373 10662633 A G); (6374 10662702 C T); (6375 10662705 C T); (6376 10663157 G A); (6377
10663582 A G); (6378 10663900 G A); (6379 10664543 A C); (6380 10664761 C A); (6381 10665264 T
C); (6382 10666335 TTATA T); (6383 10667066 G A); (6384 10668378 A AT); (6385 10668521 G A); (6386
10668522 G A); (6387 10668559 T C); (6388 10668790 G A); (6389 10668831 A G); (6390 10669378 C
T); (6391 10669415 T G); (6392 10669659 T G); (6393 10669665 A G); (6394 10669932 C G); (6395
10670511 T C); (6396 10670900 T C); (6397 10670902 G A); (6398 10671206 T C); (6399 10671548 C
T); (6400 10671698 T G); (6401 10671848 GTT G); (6402 10672536 G T); (6403 10673038 A C); (6404
10673068 C G); (6405 10673594 G A); (6406 10674057 C T); (6407 10674186 C T); (6408 10675176 G
A); (6409 10675503 A C); (6410 10675636 G T); (6411 10676141 C G); (6412 10676196 T C); (6413
10676368 A C); (6414 10676509 G A); (6415 10676561 C T); (6416 10676574 C T); (6417 10676809 C
T); (6418 10676861 G A); (6419 10677087 T C); (6420 10677121 A T); (6421 10677171 C T); (6422
10677179 C CA); (6423 10677605 C T); (6424 10678436 A AT); (6425 10678548 CA C); (6426 10679411 A
T); (6427 10679430 G GCAC); (6428 10679591 A G); (6429 10679659 T TA); (6430 10680842 T C); (6431
10680984 G A); (6432 10681076 C T); (6433 10681126 T C); (6434 10681519 T C); (6435 10681755 A
G); (6436 10682293 T G); (6437 10682513 G A); (6438 10682797 C T); (6439 10682889 G C); (6440
10683075 A G); (6441 10683130 A G); (6442 10683143 G A); (6443 10683639 G A); (6444 10683880 C
T); (6445 10683892 T C); (6446 10683907 A G); (6447 10683952 T G); (6448 10683954 C T); (6449
10683989 T C); (6450 10684031 G A); (6451 10684114 A G); (6452 10684149 A G); (6453 10684171 T
G); (6454 10684333 A G); (6455 10684414 G C); (6456 10684518 A G); (6457 10684589 A G); (6458
10684729 T A); (6459 10684860 G A); (6460 10684959 A C); (6461 10685192 T C); (6462 10685306 G
A); (6463 10685568 C T); (6464 10685648 C T); (6465 10685865 C A); (6466 10685985 T C); (6467
10686078 C T); (6468 10686266 A G); (6469 10686427 C T); (6470 10687088 G A); (6471 10687597 G
C); (6472 10687756 C A); (6473 10688381 G A); (6474 10688410 G A); (6475 10688506 G GA); (6476
10688628 T C); (6477 10688733 G T); (6478 10688814 G A); (6479 10688817 G A); (6480 10688995 C
T); (6481 10689039 A G); (6482 10689412 G A); (6483 10689669 T A); (6484 10689877 G A); (6485
10690022 C T); (6486 10690105 G A); (6487 10690129 G A); (6488 10690316 G A); (6489 10690379 C
T); (6490 10690400 T C); (6491 10691006 C T); (6492 10691129 T G); (6493 10691318 G A); (6494
10691825 TA T); (6495 10691928 T A); (6496 10692752 T TA); (6497 10693866 T A); (6498 10693867 C
G); (6499 10693957 AT A); (6500 10694160 A G); (6501 10694213 A C); (6502 10694334 A AT); (6503
10694576 A T); (6504 10694590 AAACATAAATTAG A); (6505 10694845 C T); (6506 10694897 G A); (6507
10694910 G A); (6508 10695242 T A); (6509 10695331 G A); (6510 10695359 GA G); (6511 10695483 C
T); (6512 10695517 C T); (6513 10695590 G A); (6514 10695791 CA C); (6515 10696762 C T); (6516
10696816 A G); (6517 10696882 G T); (6518 10697339 A T); (6519 10697704 C T); (6520 10698247 T
C); (6521 10699480 A T); (6522 10699482 A T); (6523 10699663 G A); (6524 10700035 C T); (6525
10700166 C T); (6526 10700678 C T); (6527 10700735 C T); (6528 10701092 C A); (6529 10701309 G
C); (6530 10702312 C A); (6531 10703663 C T); (6532 10703796 C T); (6533 10704071 C T); (6534
10705465 G A); (6535 10706446 T C); (6536 10706607 A T); (6537 10706618 G A); (6538 10706917 C
T); (6539 10707225 G GA); (6540 10707238 G C); (6541 10707270 C T); (6542 10707300 G A); (6543
10707591 C T); (6544 10707798 A G); (6545 10707876 G A); (6546 10708012 TA T); (6547 10708070 A
G); (6548 10708075 C T); (6549 10708148 A G); (6550 10708348 G A); (6551 10708515 C T); (6552
10708559 C T); (6553 10709163 C T); (6554 10709512 T C); (6555 10709531 G A); (6556 10709587 G
A); (6557 10709788 A C); (6558 10710087 C T); (6559 10710125 G A); (6560 10710138 A G); (6561
10710152 G A); (6562 10710261 T A); (6563 10710583 C T); (6564 10710603 G A); (6565 10710631 G
A); (6566 10710772 G A); (6567 10710824 A G); (6568 10710827 A C); (6569 10710896 A G); (6570
10710992 A T); (6571 10711049 A G); (6572 10711326 G A); (6573 10711603 C T); (6574 10711752 G
A); (6575 10711753 C G); (6576 10711834 G A); (6577 10711868 G A); (6578 10711983 T A); (6579
10711998 G A); (6580 10712145 T G); (6581 10712422 C T); (6582 10713076 C T); (6583 10713512 C
T); (6584 10713719 A T); (6585 10714270 T A); (6586 10714356 T G); (6587 10714360 T A); (6588
10714677 G A); (6589 10714742 A G); (6590 10714778 C T); (6591 10715056 C T); (6592 10715148 G
T); (6593 10715165 C T); (6594 10715204 G A); (6595 10715557 A G); (6596 10715673 C T); (6597
10715711 A G); (6598 10715748 G T); (6599 10715767 C G); (6600 10715768 C G); (6601 10716056 T
C); (6602 10716097 G A); (6603 10716141 G T); (6604 10716271 A G); (6605 10716304 A G); (6606
10716463 A G); (6607 10716490 G A); (6608 10716569 G T); (6609 10716595 A G); (6610 10716730 C
T); (6611 10716758 T C); (6612 10716760 C T); (6613 10716769 A G); (6614 10716770 C T); (6615
10716866 C T); (6616 10716954 C T); (6617 10717057 G A); (6618 10717357 A G); (6619 10717492 A
G); (6620 10717538 A G); (6621 10717565 C T); (6622 10717600 A G); (6623 10717648 G A); (6624
10717739 C A); (6625 10717973 G A); (6626 10718017 C T); (6627 10718148 G A); (6628 10718195 AC
A); (6629 10718228 C T); (6630 10718284 T C); (6631 10718319 G A); (6632 10718572 A G); (6633
10718886 A G); (6634 10719354 G A); (6635 10719434 C T); (6636 10719532 A G); (6637 10719602 A
G); (6638 10719717 A G); (6639 10719831 G A); (6640 10719908 C T); (6641 10719919 G A); (6642
10719984 A G); (6643 10720174 C A); (6644 10720348 T G); (6645 10720480 G A); (6646 10720679 G
A); (6647 10720864 C T); (6648 10720972 A C); (6649 10721172 T A); (6650 10721192 T C); (6651
10721333 T C); (6652 10721632 A G); (6653 10721919 G A); (6654 10722106 A C); (6655 10722122 G
A); (6656 10722127 G A); (6657 10722260 A C); (6658 10722276 G T); (6659 10722277 C T); (6660
10722303 G A); (6661 10722347 C T); (6662 10722437 C T); (6663 10722766 C T); (6664 10722792 A
G); (6665 10722852 G A); (6666 10722917 A T); (6667 10722986 A G); (6668 10722994 C T); (6669
10723280 G A); (6670 10723281 G T); (6671 10723307 T C); (6672 10723346 A G); (6673 10723350 C
G); (6674 10723417 G A); (6675 10723657 A G); (6676 10723695 G C); (6677 10723937 G A); (6678
10724656 G A); (6679 10724662 C T); (6680 10725369 A T); (6681 10725386 G A); (6682 10725500 T
C); (6683 10725764 C T); (6684 10726088 A T); (6685 10726105 C T); (6686 10726659 G A); (6687
10726688 C T); (6688 10726718 T A); (6689 10726729 G A); (6690 10726738 A G); (6691 10726831 T
C); (6692 10726854 G A); (6693 10726903 G A); (6694 10726930 G A); (6695 10726970 C T); (6696
10726976 C T); (6697 10726981 A G); (6698 10727021 G A); (6699 10727070 T C); (6700 10727113 C
G); (6701 10727398 C T); (6702 10727559 ATCT A); (6703 10727588 G A); (6704 10727592 T C); (6705
10727783 C T); (6706 10728223 C A); (6707 10728347 G T); (6708 10728495 A G); (6709 10730265 A
C); (6710 10730742 T C); (6711 10731045 C T); (6712 10731075 C A); (6713 10731185 C T); (6714
10731253 A G); (6715 10731319 C T); (6716 10731762 T C); (6717 10732178 A T); (6718 10732655 T
C); (6719 10733189 A G); (6720 10733286 A T); (6721 10733779 C T); (6722 10734989 C CTAAT); (6723
10735076 T C); (6724 10735094 T A); (6725 10735142 C T); (6726 10735308 A G); (6727 10735479 T
C); (6728 10735513 G A); (6729 10735574 G A); (6730 10735580 T C); (6731 10735772 A G); (6732
10735774 T C); (6733 10735775 G T); (6734 10735895 T C); (6735 10736077 C G); (6736 10736448 A
C); (6737 10737096 T C); (6738 10738172 C T); (6739 10738195 C T); (6740 10738508 C T); (6741
10738712 A G); (6742 10738780 C T); (6743 10738810 C T); (6744 10739001 A C); (6745 10739072 G
A); (6746 10739120 T C); (6747 10740057 T C); (6748 10740144 C T); (6749 10740508 C G); (6750
10740696 G A); (6751 10740708 G A); (6752 10740742 C A); (6753 10740997 T C); (6754 10741019 C
T); (6755 10741047 A C); (6756 10741062 G C); (6757 10741106 A G); (6758 10741177 C T); (6759
10741258 G A); (6760 10741409 A C); (6761 10741512 T C); (6762 10741744 A G); (6763 10741806 T
C); (6764 10742026 C T); (6765 10742040 G A); (6766 10742148 G A); (6767 10742241 G A); (6768
10742577 C T); (6769 10742980 G C); (6770 10743117 C T); (6771 10743146 T A); (6772 10743183 G
A); (6773 10743190 T C); (6774 10743205 C T); (6775 10743364 C G); (6776 10743871 C T); (6777
10743978 A G); (6778 10744069 G A); (6779 10744310 G A); (6780 10744977 T G); (6781 10745059 C
A); (6782 10745072 A G); (6783 10745120 G A); (6784 10745121 C T); (6785 10745503 C T); (6786
10745654 G C); (6787 10745746 C T); (6788 10746114 C T); (6789 10746173 G A); (6790 10746261 A
C); (6791 10746315 A C); (6792 10746415 C T); (6793 10746558 G A); (6794 10746814 C T); (6795
10746974 T C); (6796 10747143 C T); (6797 10747198 T C); (6798 10747422 A C); (6799 10747505 G
A); (6800 10747523 T C); (6801 10747724 C A); (6802 10747930 T C); (6803 10747999 G A); (6804
10748099 G A); (6805 10748194 A G); (6806 10748204 C G); (6807 10748250 T C); (6808 10748374 G
A); (6809 10748407 G A); (6810 10748497 C T); (6811 10748839 C T); (6812 10749126 A G); (6813
10749214 TG T); (6814 10749274 G A); (6815 10749539 C T); (6816 10749743 C G); (6817 10749817 C
A); (6818 10749874 C T); (6819 10750088 T C); (6820 10750296 C T); (6821 10750325 C T); (6822
10750540 C T); (6823 10750678 T C); (6824 10750718 T A); (6825 10750849 G C); (6826 10750856 T
G); (6827 10750857 C A); (6828 10750860 C A); (6829 10750868 G A); (6830 10750906 C T); (6831
10751154 G A); (6832 10751172 A C); (6833 10751325 C T); (6834 10751556 A C); (6835 10751661 G
A); (6836 10753178 C T); (6837 10753196 G A); (6838 10753231 C T); (6839 10753269 A G); (6840
10753276 G A); (6841 10753291 T TG); (6842 10753355 G A); (6843 10753398 C T); (6844 10753588 A
C); (6845 10753949 A G); (6846 10755040 C T); (6847 10755167 T C); (6848 10755621 C T); (6849
10755910 G A); (6850 10756179 G A); (6851 10756358 G A); (6852 10757026 C T); (6853 10757239 T
C); (6854 10757494 C T); (6855 10757588 C T); (6856 10757664 A G); (6857 10758482 G T); (6858
10758510 A G); (6859 10758630 G A); (6860 10758823 C T); (6861 10758848 C T); (6862 10758857 G
C); (6863 10758885 A C); (6864 10759467 T A); (6865 10759498 A C); (6866 10759649 T C); (6867
10759900 G A); (6868 10760046 A G); (6869 10760229 A C); (6870 10760268 C T); (6871 10760593 G
A); (6872 10760614 A G); (6873 10760705 C T); (6874 10760726 C T); (6875 10760778 T A); (6876
10760787 C T); (6877 10760841 G A); (6878 10761349 A G); (6879 10761832 G A); (6880 10761864 T
C); (6881 10761865 G T); (6882 10761869 C G); (6883 10761871 G A); (6884 10761895
TTGTCTTTGTTGTATCTGAGTTG T); (6885 10761902 T C); (6886 10761914 C T); (6887 10761995 C
T); (6888 10762001 C T); (6889 10762039 G A); (6890 10762087 A G); (6891 10762462 C T); (6892
10763067 G A); (6893 10763145 T C); (6894 10763362 C T); (6895 10763715 G A); (6896 10763722 C
T); (6897 10763802 G T); (6898 10763875 G A); (6899 10763950 G A); (6900 10764095 G T); (6901
10764104 G T); (6902 10764344 C CT); (6903 10764728 G A); (6904 10764898 C T); (6905 10765034 G
A); (6906 10765408 T A); (6907 10765540 G A); (6908 10766489 G T); (6909 10766499 G T); (6910
10767550 C A); (6911 10767805 T A); (6912 10768139 G A); (6913 10768724 TA T); (6914 10769189 G
T); (6915 10769828 A T); (6916 10769832 TA T); (6917 10769845 T C); (6918 10769850 A T); (6919
10769986 A AGTC); (6920 10770037 A AT); (6921 10770048 A G); (6922 10770240 A C); (6923 10770375
C T); (6924 10770607 C T); (6925 10770652 G A); (6926 10770751 G GT); (6927 10770778 T C); (6928
10770802 C G); (6929 10770941 C T); (6930 10771086 T A); (6931 10771167 A G); (6932 10771258 G
A); (6933 10771693 G A); (6934 10771905 C T); (6935 10771992 C T); (6936 10772066 G A); (6937
10772398 T C); (6938 10774019 C T); (6939 10774658 A G); (6940 10774720 T G); (6941 10774737 A
G); (6942 10775080 TTC T); (6943 10775639 A T); (6944 10775872 C A); (6945 10776492 A G); (6946
10776652 G A); (6947 10776674 A ATT); (6948 10776760 G A); (6949 10777065 C T); (6950 10778779 C
A); (6951 10778951 C T); (6952 10779397 C T); (6953 10779591 C T); (6954 10779924 G A); (6955
10781323 A T); (6956 10781352 TA T); (6957 10781403 A G); (6958 10781569 T G); (6959 10781978 C
T); (6960 10782461 G A); (6961 10782654 G T); (6962 10782656 A C); (6963 10782691 T C); (6964
10782862 G GA); (6965 10783878 G A); (6966 10784260 C CTTTTCTTTTATCTTATTTT); (6967 10784973 G
T); (6968 10785003 C T); (6969 10785008 A G); (6970 10785097 C T); (6971 10785121 T C); (6972
10785517 A G); (6973 10786586 G A); (6974 10787302 C T); (6975 10787341 A T); (6976 10787352 C
T); (6977 10787562 G A); (6978 10787878 G A); (6979 10788151 G A); (6980 10788318 G T); (6981
10788363 G A); (6982 10788434 C A); (6983 10789583 G A); (6984 10789892 G A); (6985 10789947 C
T); (6986 10790147 A T); (6987 10790401 A G); (6988 10790598 G A); (6989 10790910 C T); (6990
10791059 A G); (6991 10791213 G A); (6992 10791310 T C); (6993 10791380 T A); (6994 10791401 G
A); (6995 10791494 C T); (6996 10791507 C T); (6997 10791555 T C); (6998 10791581 G A); (6999
10791767 C T); (7000 10793296 A C); (7001 10793302 T C); (7002 10793312 C A); (7003 10793343 C
G); (7004 10793368 T C); (7005 10793427 A G); (7006 10793446 A G); (7007 10793476 A C); (7008
10793479 T G); (7009 10793487 T A); (7010 10793497 G A); (7011 10793499 A G); (7012 10793507 A
G); (7013 10793596 C A); (7014 10793666 A G); (7015 10793673 C T); (7016 10793805 G T); (7017
10793828 G A); (7018 10793829 T G); (7019 10793830 G T); (7020 10793847 G A); (7021 10793863 T
A); (7022 10793865 T G); (7023 10793873 A G); (7024 10793874 G A); (7025 10793914 A G); (7026
10794030 A AT); (7027 10794031 G A); (7028 10794061 G A); (7029 10794124 A G); (7030 10794172 T
C); (7031 10794173 T A); (7032 10794477 A ATTTTT); (7033 10794655 A C); (7034 10794977 C T); (7035
10796037 C T); (7036 10796648 TG T); (7037 10798069 C T); (7038 10798090 G A); (7039 10798609 G
T); (7040 10798827 A G); (7041 10799013 G A); (7042 10799081 T C); (7043 10799206 TA T); (7044
10799247 T C); (7045 10799313 G A); (7046 10799348 G A); (7047 10799391 A G); (7048 10799424 C
T); (7049 10799550 T G); (7050 10799558 A G); (7051 10799570 A G); (7052 10799571 T C); (7053
10799578 G A); (7054 10799580 C T); (7055 10799592 A G); (7056 10799598 C T); (7057 10799604 A
G); (7058 10799619 A T); (7059 10799637 C T); (7060 10799736 T C); (7061 10799739 T C); (7062
10799752 A G); (7063 10799757 T G); (7064 10801092 G T); (7065 10801140 C T); (7066 10801149 T
C); (7067 10801173 C T); (7068 10801182 A G); (7069 10801236 T A); (7070 10801775 A G); (7071
10801830 A G); (7072 10801866 T C); (7073 10802064 A G); (7074 10802127 G A); (7075 10803945 A
G); (7076 10804074 T G); (7077 10804129 C T); (7078 10804264 C T); (7079 10804337 T G); (7080
10804367 T C); (7081 10804392 A G); (7082 10805745 G C); (7083 10805944 A C); (7084 10805963 T
A); (7085 10806224 C T); (7086 10806294 G A); (7087 10807195 A T); (7088 10807197 G
GACTTGGTCCATGCCGAAT); (7089 10807380 A G); (7090 10807467 A G); (7091 10808141 C G); (7092
10808243 G T); (7093 10808562 C T); (7094 10808756 T C); (7095 10809134 G A); (7096 10809630 C
T); (7097 10809634 A G); (7098 10809781 A G); (7099 10809837 T C); (7100 10809939 G A); (7101
10810147 T C); (7102 10811973 G T); (7103 10812074 T C); (7104 10812201 C T); (7105 10812309 G
A); (7106 10812350 C T); (7107 10812449 G A); (7108 10812477 A G); (7109 10812511 G A); (7110
10813051 T C); (7111 10813303 C T); (7112 10813592 C T); (7113 10813850 C T); (7114 10813985 T
C); (7115 10814222 A G); (7116 10815126 G A); (7117 10815190 C T); (7118 10815242 T TCTTTTC); (7119
10815598 G A); (7120 10815620 C T); (7121 10816252 C G); (7122 10817292 G C); (7123 10817592 G
A); (7124 10817624 G A); (7125 10817703 T C); (7126 10817883 T C); (7127 10818106 C T); (7128
10818549 G T); (7129 10818591 C A); (7130 10818729 G T); (7131 10818783 G A); (7132 10819847 C
A); (7133 10819853 G A); (7134 10819986 T C); (7135 10820187 C T); (7136 10820375 AT A); (7137
10822204 C T); (7138 10822896 G A); (7139 10823141 C T); (7140 10823256 C T); (7141 10823433 A
G); (7142 10823605 C T); (7143 10823640 G A); (7144 10823737 A G); (7145 10823913 T A); (7146
10823931 C T); (7147 10824574 G GT); (7148 10824717 C T); (7149 10824823 C T); (7150 10824905 G
A); (7151 10824950 G T); (7152 10824958 G T); (7153 10825068 G A); (7154 10825244 T C); (7155
10825247 G A); (7156 10825377 T G); (7157 10825583 C T); (7158 10826277 T C); (7159 10826593 C
T); (7160 10826600 G A); (7161 10826715 C T); (7162 10826896 G A); (7163 10826929 G A); (7164
10828353 G A); (7165 10828414 C T); (7166 10828508 G A); (7167 10829215 A G); (7168 10830347 C
A); (7169 10831333 C T); (7170 10831409 A G); (7171 10831433 C T); (7172 10831533 C T); (7173
10831660 T C); (7174 10831685 C T); (7175 10832017 C T); (7176 10832133 C T); (7177 10832260 C
T); (7178 10832300 G A); (7179 10832550 T C); (7180 10832584 G A); (7181 10833043 G A); (7182
10833393 T C); (7183 10833575 G A); (7184 10833609 A G); (7185 10834108 C T); (7186 10834339 C
A); (7187 10834376 T C); (7188 10834589 C CT); (7189 10834770 G A); (7190 10835023 A G); (7191
10835184 C T); (7192 10835276 C A); (7193 10835888 G A); (7194 10837791 T TA); (7195 10838232 A
G); (7196 10840073 CTTA C); (7197 10840542 C T); (7198 10841146 T C); (7199 10841244 G A); (7200
10841287 C T); (7201 10841351 C T); (7202 10841521 A G); (7203 10841552 A G); (7204 10841832 C
T); (7205 10842004 G A); (7206 10842294 A G); (7207 10843136 G A); (7208 10844045 T G); (7209
10844929 G A); (7210 10845880 C T); (7211 10846061 T G); (7212 10846449 C T); (7213 10847991 A
T); (7214 10848542 A T); (7215 10848664 T G); (7216 10849715 C G); (7217 10850945 T C); (7218
10850956 A G); (7219 10851072 A T); (7220 10851113 A T); (7221 10851710 G A); (7222 10851725 G
A); (7223 10851986 G A); (7224 10851987 C T); (7225 10852013 C T); (7226 10852201 A T); (7227
10852345 G A); (7228 10852745 T C); (7229 10853085 A AT); (7230 10853364 C T); (7231 10854404 G
A); (7232 10855281 TA T); (7233 10855466 T A); (7234 10855549 G A); (7235 10855609 G T); (7236
10855610 A T); (7237 10855767 TACTAAGTAAAACA T); (7238 10855813 A C); (7239 10857622 C
T); (7240 10857663 C A); (7241 10857911 T TA); (7242 10857990 C A); (7243 10858044 G A); (7244
10858090 G GT); (7245 10858096 CA C); (7246 10858115 G A); (7247 10858146 C T); (7248 10858198 G
A); (7249 10858233 C CA); (7250 10858436 G A); (7251 10858455 G C); (7252 10858504 T C); (7253
10858657 T G); (7254 10858732 C G); (7255 10858960 G A); (7256 10858971 C T); (7257 10858994 T
C); (7258 10858998 G C); (7259 10859013 A G); (7260 10859015 T A); (7261 10859067 C T); (7262
10859117 C T); (7263 10859304 A G); (7264 10859330 C T); (7265 10859573 G T); (7266 10859687 T
A); (7267 10859744 T TA); (7268 10859755 G A); (7269 10859870 A T); (7270 10860117 A G); (7271
10860118 G T); (7272 10860132 T C); (7273 10860174 G A); (7274 10860253 G A); (7275 10860318 G
A); (7276 10861973 A G); (7277 10862100 C T); (7278 10863033 T G); (7279 10864436 C
CTACCTCACTTCACA); (7280 10864540 C A); (7281 10864643 G C); (7282 10864648 C T); (7283 10864926
CT); (7284 10864980 A G); (7285 10865113 A T); (7286 10865224 T C); (7287 10865229 A T); (7288
10865787 CAA C); (7289 10865949 G A); (7290 10865950 T G); (7291 10866055 T A); (7292 10866142 C
T); (7293 10866171 G A); (7294 10866239 A G); (7295 10866240 C T); (7296 10866246 G T); (7297
10866262 C A); (7298 10866349 T C); (7299 10866403 G A); (7300 10866419 C T); (7301 10866483 G
C); (7302 10866493 A G); (7303 10866521 G A); (7304 10866856 C T); (7305 10866943 T G); (7306
10867054 C T); (7307 10867072 C G); (7308 10867158 G A); (7309 10867232 T G); (7310 10867286 G
A); (7311 10867294 C T); (7312 10867366 A G); (7313 10867536 G A); (7314 10867767 C T); (7315
10867843 T C); (7316 10867920 T C); (7317 10867950 C T); (7318 10868018 A G); (7319 10868138 G
C); (7320 10868149 T C); (7321 10868154 G T); (7322 10868159 G T); (7323 10868183 T C); (7324
10868268 G A); (7325 10868360 A G); (7326 10868494 A G); (7327 10868535 C T); (7328 10868596 C
A); (7329 10868663 C T); (7330 10868830 G C); (7331 10868906 T C); (7332 10869105 G A); (7333
10869130 G C); (7334 10869162 C A); (7335 10869254 G A); (7336 10869377 G A); (7337 10869519 AG
A); (7338 10869763 A G); (7339 10869789 C T); (7340 10869842 A G); (7341 10869844 C T); (7342
10869894 GA G); (7343 10871113 G A); (7344 10871240 T C); (7345 10872895 C T); (7346 10872960 C
T); (7347 10873039 C T); (7348 10873081 A G); (7349 10873095 C A); (7350 10873096 T C); (7351
10873343 T A); (7352 10873458 C T); (7353 10873473 A AT); (7354 10873526 A T); (7355 10873640 C
A); (7356 10873895 G A); (7357 10873921 T C); (7358 10874212 T C); (7359 10874227 C G); (7360
10874231 A G); (7361 10874254 G A); (7362 10874265 C T); (7363 10874493 G C); (7364 10874721 A
G); (7365 10874770 C G); (7366 10874789 C T); (7367 10874964 A G); (7368 10874998 A AT); (7369
10875027 C T); (7370 10875079 G A); (7371 10875110 C T); (7372 10875131 AT A); (7373 10875143 C
CA); (7374 10875181 C T); (7375 10875235 G T); (7376 10875321 A AT); (7377 10875562 G T); (7378
10875603 G A); (7379 10877412 T G); (7380 10877475 AAGTTGTTTTACTT A); (7381 10877918 T
C); (7382 10877930 G A); (7383 10877948 G A); (7384 10878077 A G); (7385 10878106 C T); (7386
10878185 T C); (7387 10878213 T C); (7388 10878287 A G); (7389 10878438 T A); (7390 10878862 T
G); (7391 10879412 T A); (7392 10879649 C A); (7393 10879720 A G); (7394 10879760 G C); (7395
10879908 TTATA T); (7396 10879979 A T); (7397 10880001 A T); (7398 10880021 T G); (7399 10880254
C T); (7400 10880260 T TGACATGACATTGTAGG); (7401 10880271 A T); (7402 10880313 G A); (7403
10880321 T G); (7404 10880324 T G); (7405 10880369 C T); (7406 10880384 T G); (7407 10880408 T
A); (7408 10880415 C CTG); (7409 10880425 T A); (7410 10880427 A G); (7411 10880470 A G); (7412
10880521 G T); (7413 10880528 T A); (7414 10880576 G T); (7415 10880684 A G); (7416 10880948 T
G); (7417 10881334 G C); (7418 10881474 A G); (7419 10881586 T C); (7420 10881749 C A); (7421
10881948 A C); (7422 10882677 A G); (7423 10882680 A G); (7424 10882689 T C); (7425 10882748 A
G); (7426 10882808 G A); (7427 10882878 G T); (7428 10882896 C T); (7429 10883000 G A); (7430
10883156 C T); (7431 10883201 A G); (7432 10883324 T G); (7433 10883383 C T); (7434 10883517 G
A); (7435 10883522 A G); (7436 10883705 G A); (7437 10883799 G A); (7438 10884172 T G); (7439
10884726 A G); (7440 10884777 C T); (7441 10884863 T C); (7442 10884981 A T); (7443 10885127 A
G); (7444 10885728 C T); (7445 10885790 T C); (7446 10886125 C T); (7447 10886248 A C); (7448
10886413 G A); (7449 10886487 G A); (7450 10886492 C T); (7451 10886511 A G); (7452 10886558 CT
C); (7453 10886596 T A); (7454 10886940 T C); (7455 10886943 T G); (7456 10887031 A T); (7457
10887143 G A); (7458 10887273 C T); (7459 10887310 T A); (7460 10887714 G A); (7461 10887793 A
T); (7462 10888087 G A); (7463 10888214 G C); (7464 10888410 A G); (7465 10888646 T C); (7466
10888729 T C); (7467 10888926 A G); (7468 10888969 AC A); (7469 10888977 C T); (7470 10888992 G
T); (7471 10889065 G A); (7472 10889084 T A); (7473 10889113 G A); (7474 10889159 C T); (7475
10889291 G A); (7476 10889302 C T); (7477 10889388 A C); (7478 10889446 G A); (7479 10889546 T
C); (7480 10889678 G A); (7481 10891278 G A); (7482 10891290 G A); (7483 10891302 G T); (7484
10891439 G A); (7485 10891442 A G); (7486 10891598 A G); (7487 10891612 T C); (7488 10891971 A
G); (7489 10892161 A G); (7490 10892215 C A); (7491 10892326 G A); (7492 10892592 C A); (7493
10892612 C T); (7494 10892621 T C); (7495 10892735 A G); (7496 10892762 G T); (7497 10892843 C
T); (7498 10892926 C T); (7499 10892960 T C); (7500 10892975 A G); (7501 10892980 T C); (7502
10892981 A G); (7503 10893019 A G); (7504 10893282 C T); (7505 10893468 G A); (7506 10893575 C
G); (7507 10893674 T C); (7508 10893729 A G); (7509 10893805 T C); (7510 10893843 C T); (7511
10893953 A G); (7512 10894195 G A); (7513 10894246 T C); (7514 10894268 C T); (7515 10894331 C
T); (7516 10894360 C T); (7517 10894397 A G); (7518 10894447 A T); (7519 10894575 A G); (7520
10894706 A G); (7521 10894735 C T); (7522 10895235 T C); (7523 10895294 G A); (7524 10895322 G
A); (7525 10896266 A G); (7526 10896318 T C); (7527 10896393 A G); (7528 10896505 G T); (7529
10896525 C T); (7530 10896604 A G); (7531 10896782 G A); (7532 10896885 A G); (7533 10896953 T
C); (7534 10896988 T C); (7535 10897118 G A); (7536 10897141 C T); (7537 10897206 G T); (7538
10897218 G A); (7539 10897273 C T); (7540 10897308 T C); (7541 10897360 C T); (7542 10897366 T
G); (7543 10897413 C T); (7544 10898131 T C); (7545 10898292 A C); (7546 10898310 G A); (7547
10898313 T C); (7548 10898315 A G); (7549 10898494 A G); (7550 10898970 C T); (7551 10899057 TA
T); (7552 10899144 T C); (7553 10899212 G A); (7554 10899257 T C); (7555 10899300 T C); (7556
10899363 GT G); (7557 10899547 C T); (7558 10899563 C T); (7559 10899626 G A); (7560 10899731 G
C); (7561 10899818 G C); (7562 10899830 G A); (7563 10899854 T C); (7564 10899905 G C); (7565
10899907 G GC); (7566 10900005 A G); (7567 10900251 G A); (7568 10900401 T A); (7569 10900632 A
C); (7570 10900689 A G); (7571 10900757 A T); (7572 10900818 A G); (7573 10900835 A G); (7574
10900894 A T); (7575 10900929 A G); (7576 10901117 G A); (7577 10901191 T C); (7578 10901306 T
C); (7579 10901307 G A); (7580 10901318 T A); (7581 10901330 A G); (7582 10901364 G A); (7583
10901384 T G); (7584 10901416 G T); (7585 10901551 G A); (7586 10901563 T C); (7587 10901605 T
C); (7588 10901620 T C); (7589 10901649 G A); (7590 10901651 C T); (7591 10901726 C T); (7592
10901887 T C); (7593 10901910 A G); (7594 10901913 T C); (7595 10901923 G A); (7596 10901952 A
T); (7597 10902151 T C); (7598 10902249 C G); (7599 10902368 A G); (7600 10902387 C T); (7601
10902559 T C); (7602 10902613 G T); (7603 10902678 T G); (7604 10902795 A G); (7605 10902899 T
C); (7606 10902904 G A); (7607 10902957 G A); (7608 10902980 C T); (7609 10903066 C T); (7610
10903103 T C); (7611 10903112 T C); (7612 10903174 G A); (7613 10903185 C T); (7614 10903236 C
A); (7615 10903286 C T); (7616 10903321 G A); (7617 10903474 T TA); (7618 10903900 T C); (7619
10903996 G C); (7620 10904010 T C); (7621 10904043 T A); (7622 10904080 C T); (7623 10904595 G
C); (7624 10905122 G T); (7625 10905156 A G); (7626 10905540 T C); (7627 10905840 C T); (7628
10906084 T TC); (7629 10906401 G C); (7630 10906535 T C); (7631 10906589 G GA); (7632 10906674 T
C); (7633 10906721 AACT A); (7634 10906938 T C); (7635 10907123 G T); (7636 10907161 C T); (7637
10907207 A C); (7638 10907220 G A); (7639 10907270 A AT); (7640 10907493 A G); (7641 10907544 C
T); (7642 10907547 G A); (7643 10907595 T C); (7644 10907665 T A); (7645 10907666 T A); (7646
10907684 T C); (7647 10907795 C T); (7648 10907830 T C); (7649 10907844 G A); (7650 10907888 T
C); (7651 10907913 A G); (7652 10907918 A G); (7653 10907978 A C); (7654 10908073 C A); (7655
10908106 C T); (7656 10908254 T C); (7657 10908417 T A); (7658 10908826 A G); (7659 10909125 A
G); (7660 10909216 G T); (7661 10909270 C T); (7662 10909272 T C); (7663 10909303 A C); (7664
10909335 G A); (7665 10909594 C A); (7666 10909794 A G); (7667 10909819 G A); (7668 10909821 T
C); (7669 10909843 A G); (7670 10909863 C T); (7671 10909899 C T); (7672 10910214 T C); (7673
10910352 A T); (7674 10910362 A G); (7675 10910434 T A); (7676 10911047 G A); (7677 10911230 A
C); (7678 10911459 G A); (7679 10911637 G C); (7680 10911669 T C); (7681 10911701 C T); (7682
10911740 C T); (7683 10911800 A C); (7684 10911950 A C); (7685 10912005 T C); (7686 10912169 T
C); (7687 10912222 G A); (7688 10912268 A G); (7689 10912546 C CGATGAATTATTTTCCATTGTCT); (7690
10912548 C G); (7691 10912549 C A); (7692 10912634 G A); (7693 10912676 G A); (7694 10912696 T
C); (7695 10912761 T C); (7696 10912852 A G); (7697 10912999 C T); (7698 10913137 C T); (7699
10913212 C T); (7700 10913332 G T); (7701 10913337 G T); (7702 10913416 T C); (7703 10913475 T
G); (7704 10913477 T C); (7705 10913528 C T); (7706 10913653 A T); (7707 10915247 T G); (7708
10915269 G A); (7709 10915375 T C); (7710 10915416 G T); (7711 10915461 G A); (7712 10915513 A
G); (7713 10915555 A G); (7714 10915585 A G); (7715 10915624 C T); (7716 10915637 A G); (7717
10915672 G C); (7718 10915678 G C); (7719 10915705 C T); (7720 10915846 T A); (7721 10915859 A
G); (7722 10915902 T G); (7723 10916052 T C); (7724 10916088 G A); (7725 10916122 A G); (7726
10916131 T C); (7727 10916166 T C); (7728 10916224 G A); (7729 10916276 C T); (7730 10916300 C
T); (7731 10916302 A G); (7732 10916320 C T); (7733 10916326 G A); (7734 10916338 G T); (7735
10916401 G A); (7736 10916442 C T); (7737 10916501 G A); (7738 10916502 A C); (7739 10916518 G
A); (7740 10916521 A G); (7741 10916549 T C); (7742 10916595 C G); (7743 10916602 T C); (7744
10916617 A G); (7745 10916626 T C); (7746 10916660 G T); (7747 10916662 T C); (7748 10916742 G
A); (7749 10916765 C T); (7750 10916775 G A); (7751 10916813 C T); (7752 10916850 G T); (7753
10916852 A C); (7754 10916866 T C); (7755 10917034 T C); (7756 10917071 C G); (7757 10917078 G
A); (7758 10917122 A G); (7759 10917141 G A); (7760 10917163 C G); (7761 10917195 A G); (7762
10917208 T A); (7763 10917285 G A); (7764 10917290 A G); (7765 10917292 C T); (7766 10917388 T
A); (7767 10917405 G A); (7768 10917523 A G); (7769 10917531 C T); (7770 10917542 T C); (7771
10917569 T C); (7772 10917590 A G); (7773 10917620 A G); (7774 10917661 A G); (7775 10917677 T
C); (7776 10917697 A G); (7777 10917708 T C); (7778 10917721 A G); (7779 10917732 T G); (7780
10917733 G C); (7781 10917744 T C); (7782 10917765 T C); (7783 10917853 G A); (7784 10917884 T
C); (7785 10917915 T G); (7786 10918309 G A); (7787 10918448 G T); (7788 10918748 A T); (7789
10918783 G A); (7790 10918820 T C); (7791 10919233 A C); (7792 10919480 A C); (7793 10919552 A
G); (7794 10920209 TA T); (7795 10920379 C A); (7796 10920492 C A); (7797 10921864 A T); (7798
10922082 A G); (7799 10923465 C CAG); (7800 10924016 G C); (7801 10924441 T TA); (7802 10924947 T
C); (7803 10925658 G A); (7804 10925801 C T); (7805 10926102 A G); (7806 10926264 C T); (7807
10926550 C T); (7808 10926551 A G); (7809 10926571 A T); (7810 10926905 CA C); (7811 10928276 G
A); (7812 10928638 T C); (7813 10928753 T C); (7814 10928755 T C); (7815 10928757 T C); (7816
10928839 G A); (7817 10928947 G A); (7818 10929262 T C); (7819 10929294 A G); (7820 10929360 G
A); (7821 10929962 T G); (7822 10930200 G A); (7823 10930639 G A); (7824 10930769 A G); (7825
10930976 T C); (7826 10931246 AT A); (7827 10931338 G A); (7828 10931347 C T); (7829 10931423 T
C); (7830 10931778 A G); (7831 10931873 TA T); (7832 10932060 C T); (7833 10932128 T A); (7834
10932204 CA C); (7835 10932553 T G); (7836 10932913 A C); (7837 10932923 C T); (7838 10933030 C
T); (7839 10933110 C T); (7840 10933253 G A); (7841 10933390 G C); (7842 10933445 T C); (7843
10933479 G A); (7844 10933810 A G); (7845 10934159 A G); (7846 10934300 T G); (7847 10934380 A
G); (7848 10934420 C CG); (7849 10934485 G A); (7850 10935023 C T); (7851 10935252 A C); (7852
10935310 C T); (7853 10935949 T G); (7854 10935958 A G); (7855 10936169 G T); (7856 10936544 T
C); (7857 10936582 G A); (7858 10936616 G A); (7859 10937639 T G); (7860 10938028 C T); (7861
10938045 G A); (7862 10938471 G A); (7863 10938574 A G); (7864 10939100 G T); (7865 10939118 C
A); (7866 10939492 T G); (7867 10939617 C T); (7868 10939828 G A); (7869 10939839 C G); (7870
10939979 G A); (7871 10940016 C T); (7872 10940020 G A); (7873 10940192 C T); (7874 10940205 A
G); (7875 10940381 C T); (7876 10940561 C T); (7877 10940628 T G); (7878 10940800 A G); (7879
10941067 C G); (7880 10941155 T C); (7881 10941560 G A); (7882 10941597 C T); (7883 10941629 C
T); (7884 10941918 A ATCT); (7885 10942059 C T); (7886 10942107 A G); (7887 10942470 C T); (7888
10942886 G A); (7889 10943050 C T); (7890 10943076 G T); (7891 10943170 C T); (7892 10943423 T
C); (7893 10943506 G GTA); (7894 10943546 C G); (7895 10943667 G A); (7896 10944441 C A); (7897
10945102 T C); (7898 10945185 G A); (7899 10945732 C T); (7900 10946097 C T); (7901 10946459 C
T); (7902 10946700 A G); (7903 10946818 A T); (7904 10947714 C CT); (7905 10947789 C T); (7906
10948931 C A); (7907 10948974 C T); (7908 10949267 A C); (7909 10949434 G T); (7910 10949594 C
T); (7911 10949657 G T); (7912 10950073 A G); (7913 10950083 G A); (7914 10950168 T C); (7915
10951974 G A); (7916 10952005 G A); (7917 10952390 G T); (7918 10952697 A T); (7919 10953175 G
T); (7920 10954327 A G); (7921 10954623 G A); (7922 10955236 C A); (7923 10956316 G C); (7924
10957399 C T); (7925 10958677 CT C); (7926 10958876 C CT); (7927 10959154 T C); (7928 10960650 A
T); (7929 10960770 C T); (7930 10960997 A AAC); (7931 10961033 C T); (7932 10961133 C G); (7933
10961138 T C); (7934 10961155 A G); (7935 10961176 T C); (7936 10961238 C T); (7937 10961688 T
A); (7938 10961942 C T); (7939 10962279 C T); (7940 10962449 G A); (7941 10962477 G A); (7942
10962520 T C); (7943 10962603 G A); (7944 10962623 C T); (7945 10962697 T A); (7946 10963127 T
C); (7947 10963173 G C); (7948 10963350 A G); (7949 10963597 G C); (7950 10963852 G A); (7951
10964006 C CTATT); (7952 10964727 A T); (7953 10964744 G A); (7954 10965873 T G); (7955 10967208
G T); (7956 10967260 T C); (7957 10967585 C T); (7958 10967724 G A); (7959 10968866 A AT); (7960
10969061 T A); (7961 10970097 G A); (7962 10971348 T C); (7963 10971364 G T); (7964 10971380 T
G); (7965 10971484 T A); (7966 10971548 T TA); (7967 10972163 A AT); (7968 10972280 A T); (7969
10972327 TA T); (7970 10972356 C G); (7971 10972779 T TATG); (7972 10973623 G A); (7973 10973730
G A); (7974 10973756 T C); (7975 10973767 T C); (7976 10973772 G A); (7977 10973800 C G); (7978
10973871 A G); (7979 10973944 C T); (7980 10973956 A C); (7981 10974007 T C); (7982 10974020 C
A); (7983 10974081 T C); (7984 10974088 G C); (7985 10974252 C T); (7986 10974288 A AGT); (7987
10974397 C T); (7988 10974426 G A); (7989 10974463 G A); (7990 10974498 A G); (7991 10974560 C
T); (7992 10974600 TTTTAG T); (7993 10974639 A G); (7994 10974815 G A); (7995 10975225 A T); (7996
10975444 T C); (7997 10975610 AT A); (7998 10976802 A T); (7999 10976819 A AGC); (8000 10976918 T
C); (8001 10977017 T C); (8002 10977248 C T); (8003 10977302 T C); (8004 10977303 C T); (8005
10977319 G A); (8006 10977424 T A); (8007 10977524 A T); (8008 10977557 A G); (8009 10977583 T
TAA); (8010 10977694 C T); (8011 10977840 A G); (8012 10977856 T C); (8013 10977893 A C); (8014
10977946 T C); (8015 10978310 A G); (8016 10978493 G A); (8017 10978504 G A); (8018 10978674 G
A); (8019 10978691 G A); (8020 10978699 T C); (8021 10978791 T C); (8022 10978972 T C); (8023
10979302 T A); (8024 10979814 A AG); (8025 10980015 T A); (8026 10980193 T C); (8027 10980369 A
T); (8028 10980384 T C); (8029 10980462 G T); (8030 10980583 G A); (8031 10980673 A T); (8032
10980911 G A); (8033 10981080 T C); (8034 10981112 A G); (8035 10981118 G C); (8036 10981241 C
A); (8037 10981484 A T); (8038 10981571 C T); (8039 10981704 T C); (8040 10982259 G C); (8041
10982402 A T); (8042 10982412 A G); (8043 10982417 G A); (8044 10982519 T G); (8045 10982555 C
T); (8046 10982588 G C); (8047 10982647 T C); (8048 10982669 C T); (8049 10982675 C T); (8050
10982706 A G); (8051 10982832 G A); (8052 10983931 A G); (8053 10984060 T G); (8054 10984107 A
G); (8055 10984214 T A); (8056 10984224 A C); (8057 10985123 A C); (8058 10985179 G A); (8059
10985191 G A); (8060 10985201 T C); (8061 10985213 A G); (8062 10985237 C T); (8063 10985252 C
T); (8064 10985274 G A); (8065 10985310 A G); (8066 10985341 T C); (8067 10985612 T C); (8068
10985891 A G); (8069 10985901 C T); (8070 10985914 G C); (8071 10985990 A C); (8072 10986074 A
G); (8073 10986158 G A); (8074 10987015 C T); (8075 10987050 G A); (8076 10987102 T C); (8077
10987202 T C); (8078 10987380 T G); (8079 10987397 G A); (8080 10987446 C T); (8081 10987490 C
T); (8082 10987564 TA T); (8083 10987797 A G); (8084 10988359 G T); (8085 10988630 C T); (8086
10989249 A G); (8087 10989394 T G); (8088 10989528 G A); (8089 10989543 C T); (8090 10990051 G
C); (8091 10990497 T C); (8092 10990516 C T); (8093 10991111 C T); (8094 10991141 G C); (8095
10991157 A G); (8096 10991508 G A); (8097 10991652 G A); (8098 10992023 T C); (8099 10992166 C
T); (8100 10992290 T C); (8101 10992333 A G); (8102 10992861 A G); (8103 10992908 C A); (8104
10993313 G A); (8105 10993386 C T); (8106 10993414 G A); (8107 10993532 G A); (8108 10993535 G
T); (8109 10993574 C T); (8110 10993629 G T); (8111 10993630 A T); (8112 10993684 G A); (8113
10993767 C A); (8114 10993823 G T); (8115 10994405 C T); (8116 10994792 T G); (8117 10995483 C
T); (8118 10995607 G A); (8119 10995621 T C); (8120 10996046 T G); (8121 10996086 G T); (8122
10996196 T C); (8123 10996229 G A); (8124 10997183 A C); (8125 10997224 A G); (8126 10997239 A
G); (8127 10997272 GA G); (8128 10997454 T G); (8129 10997747 T C); (8130 10997968 G T); (8131
10997997 A G); (8132 10998057 T C); (8133 10998222 A G); (8134 10998404 GTGA G); (8135 10998438
A G); (8136 10998979 A C); (8137 10999189 T G); (8138 10999199 A G); (8139 10999218 T C); (8140
10999299 T C); (8141 10999348 G A); (8142 10999501 G A); (8143 10999667 G A); (8144 11000193 C
T); (8145 11000338 G A); (8146 11000651 A G); (8147 11001127 C T); (8148 11001296 G T); (8149
11001573 A T); (8150 11001786 A T); (8151 11001949 T C); (8152 11002106 A G); (8153 11002272 T
G); (8154 11002492 A G); (8155 11002586 A G); (8156 11002746 T TAAG); (8157 11003231 A T); (8158
11003611 T G); (8159 11003859 G C); (8160 11003886 GC G); (8161 11003991 A T); (8162 11004326 A
G); (8163 11004341 G A); (8164 11004513 G A); (8165 11005079 T C); (8166 11005349 C T); (8167
11005442 A G); (8168 11005753 T C); (8169 11005820 C G); (8170 11006044 G A); (8171 11006103 T
C); (8172 11006142 A C); (8173 11006143 T A); (8174 11006149 T C); (8175 11006336 A T); (8176
11006417 T G); (8177 11006454 C A); (8178 11006458 T A); (8179 11006539 C T); (8180 11006785 A
G); (8181 11006811 C T); (8182 11006839 G A); (8183 11007246 A G); (8184 11007306 G A); (8185
11010436 C T); (8186 11010641 C G); (8187 11010841 C T); (8188 11011054 T C); (8189 11011685 G
A); (8190 11011746 G A); (8191 11012206 G A); (8192 11012340 A C); (8193 11012463 C T); (8194
11012709 C G); (8195 11012934 T TA); (8196 11012947 A G); (8197 11013141 A G); (8198 11013173 G
A); (8199 11013222 G A); (8200 11013391 G A); (8201 11013849 A G); (8202 11014227 A C); (8203
11014374 G A); (8204 11014517 C T); (8205 11014763 G A); (8206 11015388 T C); (8207 11015501 C
T); (8208 11015633 C G); (8209 11016008 T C); (8210 11016087 G A); (8211 11016284 A T); (8212
11016353 G A); (8213 11016978 C T); (8214 11017058 A G); (8215 11017106 C T); (8216 11017466 T
C); (8217 11017572 A G); (8218 11017637 T C); (8219 11017709 C T); (8220 11017882 A G); (8221
11017971 C A); (8222 11018108 G A); (8223 11018446 C A); (8224 11018569 G T); (8225 11018735 T
G); (8226 11018840 A G); (8227 11019254 G A); (8228 11019351 T C); (8229 11019453 G A); (8230
11019457 C T); (8231 11019512 G A); (8232 11019699 C T); (8233 11019718 C G); (8234 11019803 C
T); (8235 11019826 C T); (8236 11019916 C T); (8237 11020165 T G); (8238 11020179 C T); (8239
11020276 C T); (8240 11020407 C T); (8241 11020836 A G); (8242 11021043 A G); (8243 11021265 G
A); (8244 11021278 A G); (8245 11021336 A C); (8246 11021429 G T); (8247 11021449 T G); (8248
11021712 G A); (8249 11022093 A G); (8250 11022466 TGGGC T); (8251 11022514 G A); (8252 11022517
A C); (8253 11023094 G A); (8254 11023227 A C); (8255 11023406 C T); (8256 11023584 T G); (8257
11023669 C G); (8258 11023746 AC A); (8259 11023771 T C); (8260 11023964 A C); (8261 11024140 C
T); (8262 11024300 G A); (8263 11024308 T A); (8264 11024336 C T); (8265 11024374 T C); (8266
11024388 C T); (8267 11024677 G C); (8268 11024697 A G); (8269 11024934 C A); (8270 11025143 A
G); (8271 11025477 C T); (8272 11026543 A T); (8273 11026675 T C); (8274 11027859 C T); (8275
11028026 G GA); (8276 11028289 T A); (8277 11028292 A ATACAT); (8278 11028535 G A); (8279
11028693 T C); (8280 11028705 A C); (8281 11028867 T C); (8282 11028986 A G); (8283 11029123 A
AT); (8284 11029738 T G); (8285 11029890 T G); (8286 11030105 A G); (8287 11030559 A G); (8288
11030848 G A); (8289 11031608 C T); (8290 11031848 A T); (8291 11031963 A G); (8292 11032694 A
G); (8293 11032970 T C); (8294 11033172 T G); (8295 11033921 A G); (8296 11033968 A G); (8297
11034076 T A); (8298 11034110 G GA); (8299 11034186 A G); (8300 11034219 C T); (8301 11034259 T
C); (8302 11034313 A G); (8303 11034656 G A); (8304 11034725 C G); (8305 11035055 T C); (8306
11035546 T C); (8307 11035689 C CAACT); (8308 11035746 C T); (8309 11035911 C A); (8310 11036177
C T); (8311 11036353 A T); (8312 11036443 T C); (8313 11036499 G A); (8314 11036522 C T); (8315
11036582 G A); (8316 11036675 A G); (8317 11036896 T C); (8318 11037674 T C); (8319 11037736 T
C); (8320 11038393 A T); (8321 11038514 A C); (8322 11038522 AG A); (8323 11038801 T G); (8324
11040589 CGAATGATGGAGAA C); (8325 11040756 T A); (8326 11040935 G
GTTGTCAATATTTATTCTTGTT); (8327 11041227 G A); (8328 11041486 G A); (8329 11041511 G A); (8330
11041735 T C); (8331 11041833 C T); (8332 11041892 T C); (8333 11042361 G A); (8334 11043633 A
G); (8335 11044581 G T); (8336 11044825 G A); (8337 11044936 C T); (8338 11045092 G A); (8339
11045268 T TACTTACAGATCTTATGTA); (8340 11045901 C A); (8341 11045972 A T); (8342 11046146 C
T); (8343 11046874 A G); (8344 11047350 A G); (8345 11047427 A G); (8346 11047785 A AT); (8347
11047878 T A); (8348 11048007 C T); (8349 11048191 G A); (8350 11049022 G C); (8351 11049296 T
TTCA); (8352 11049401 A G); (8353 11049497 A G); (8354 11049601 G A); (8355 11049667 T C); (8356
11049695 C T); (8357 11049811 G A); (8358 11049921 T A); (8359 11049971 C A); (8360 11050240 T
A); (8361 11050342 AT A); (8362 11050690 G A); (8363 11051352 G GT); (8364 11051362 C T); (8365
11051560 C T); (8366 11052643 A T); (8367 11052741 A G); (8368 11052912 T G); (8369 11052989 C
T); (8370 11053036 T A); (8371 11053176 G A); (8372 11053277 T C); (8373 11053349 G A); (8374
11053452 T C); (8375 11053648 C T); (8376 11053926 G C); (8377 11053982 A G); (8378 11054064 T
C); (8379 11054159 G A); (8380 11054254 AAG A); (8381 11054585 C T); (8382 11054775 C T); (8383
11054818 T C); (8384 11055217 T TA); (8385 11055254 A G); (8386 11055713 A T); (8387 11055735 G
A); (8388 11056136 G A); (8389 11056502 G C); (8390 11056566 A G); (8391 11056717 T C); (8392
11056754 A G); (8393 11057157 C T); (8394 11057379 C T); (8395 11057937 G C); (8396 11058057 G
A); (8397 11058634 AC A); (8398 11058738 A T); (8399 11059118 C T); (8400 11059216 T G); (8401
11059243 A G); (8402 11059334 G A); (8403 11059450 G T); (8404 11059836 C T); (8405 11059931 A
G); (8406 11059939 A C); (8407 11059943 T G); (8408 11060579 A AT); (8409 11061036 A T); (8410
11061556 AT A); (8411 11063547 T A); (8412 11064065 T A); (8413 11064120 G C); (8414 11065185 C
T); (8415 11065578 G A); (8416 11065822 G A); (8417 11067270 A C); (8418 11067362 C CT); (8419
11067500 A G); (8420 11067645 TTTACCC T); (8421 11067974 T C); (8422 11069622 C T); (8423
11069782 G C); (8424 11070504 C T); (8425 11070883 C T); (8426 11070911 A G); (8427 11071345 T
C); (8428 11071525 TA T); (8429 11072006 C T); (8430 11072269 A T); (8431 11072517 A G); (8432
11073525 T G); (8433 11074398 A G); (8434 11074955 G A); (8435 11075092 C G); (8436 11075225 G
A); (8437 11076641 A G); (8438 11076726 G A); (8439 11076958 G A); (8440 11079673 G A); (8441
11079676 T C); (8442 11079688 C T); (8443 11083542 C T); (8444 11083546 G A); (8445 11083550 A
G); (8446 11083680 C T); (8447 11084287 A G); (8448 11084739 A G); (8449 11084943 T C); (8450
11085037 T C); (8451 11085274 A G); (8452 11085598 T C); (8453 11085611 C T); (8454 11085644 G
A); (8455 11085982 G A); (8456 11086010 A G); (8457 11086058 G C); (8458 11086121 T G); (8459
11086312 C A); (8460 11086318 G A); (8461 11086330 G A); (8462 11086335 T C); (8463 11086357 C
T); (8464 11086367 G A); (8465 11086369 C A); (8466 11086382 C G); (8467 11086387 C T); (8468
11086406 C T); (8469 11086408 G A); (8470 11086423 A G); (8471 11086520 C A); (8472 11086524 T
C); (8473 11086539 T C); (8474 11086559 C T); (8475 11086584 C T); (8476 11086616 T C); (8477
11086650 G A); (8478 11086729 G A); (8479 11086838 G A); (8480 11086894 G A); (8481 11087216 A
G); (8482 11087426 A C); (8483 11087452 A G); (8484 11087466 C T); (8485 11087505 C T); (8486
11087599 C T); (8487 11087861 A G); (8488 11087868 A C); (8489 11087946 T G); (8490 11088045 G
A); (8491 11088099 T C); (8492 11088222 T G); (8493 11088385 G T); (8494 11088454 G A); (8495
11088484 C T); (8496 11088879 T C); (8497 11088904 T A); (8498 11089083 T C); (8499 11089298 C
T); (8500 11089351 G A); (8501 11089529 C A); (8502 11089600 A G); (8503 11089784 T C); (8504
11089805 G A); (8505 11089971 GGA G); (8506 11090507 G A); (8507 11090860 T C); (8508 11090866 C
T); (8509 11090889 C T); (8510 11091364 T C); (8511 11091390 C T); (8512 11091452 C T); (8513
11091616 G A); (8514 11091662 A T); (8515 11091877 G A); (8516 11091907 G A); (8517 11092347 T
A); (8518 11092425 TA T); (8519 11093414 C G); (8520 11094154 A G); (8521 11095022 G A); (8522
11095900 C T); (8523 11096228 C G); (8524 11096309 A C); (8525 11096314 T A); (8526 11096609 C
A); (8527 11096987 G C); (8528 11097262 A T); (8529 11097359 G A); (8530 11097488 C A); (8531
11098028 G T); (8532 11098217 T G); (8533 11098253 T TA); (8534 11099085 G C); (8535 11099133 C
A); (8536 11099669 A T); (8537 11099670 C T); (8538 11099951 C T); (8539 11100122 C T); (8540
11100671 A G); (8541 11101252 G C); (8542 11101331 T C); (8543 11102235 T C); (8544 11102290 A
C); (8545 11102518 C G); (8546 11103535 A G); (8547 11103900 G T); (8548 11104265 G A); (8549
11104908 A G); (8550 11104924 A G); (8551 11105794 G A); (8552 11106330 A G); (8553 11106452 C
T); (8554 11106514 G A); (8555 11106634 T C); (8556 11107177 T C); (8557 11107255 T G); (8558
11107554 A C); (8559 11108291 C T); (8560 11108317 C G); (8561 11108796 A G); (8562 11108834 T
C); (8563 11108942 A G); (8564 11108956 A G); (8565 11109021 T C); (8566 11109262 T G); (8567
11110838 T A); (8568 11111038 C A); (8569 11111514 C T); (8570 11112122 A AAAT); (8571 11112303
AC A); (8572 11112486 T TA); (8573 11113539 T G); (8574 11113671 A C); (8575 11114474 T C); (8576
11115052 G A); (8577 11115447 A G); (8578 11115560 T C); (8579 11115984 G A); (8580 11116096 C
T); (8581 11116500 T A); (8582 11116685 A G); (8583 11117567 G A); (8584 11117596 T C); (8585
11117987 G A); (8586 11118382 T A); (8587 11118454 C T); (8588 11118828 T G); (8589 11118856 T
C); (8590 11118909 T C); (8591 11118926 G A); (8592 11118950 G A); (8593 11118956 G A); (8594
11118975 A T); (8595 11118983 C T); (8596 11119199 C T); (8597 11120642 AT A); (8598 11121276 A
G); (8599 11121860 A G); (8600 11122319 TG T); (8601 11124348 C T); (8602 11124508 G A); (8603
11124620 G A); (8604 11124636 C G); (8605 11124693 G C); (8606 11124694 C G); (8607 11124717 C
T); (8608 11124958 T TCA); (8609 11125592 AT A); (8610 11126821 A T); (8611 11126885 A C); (8612
11127087 T G); (8613 11127141 A G); (8614 11127830 G A); (8615 11127979 G GT); (8616 11128159 A
T); (8617 11128485 C T); (8618 11128685 T C); (8619 11128762 C T); (8620 11129873 T TA); (8621
11130166 AAG A); (8622 11130280 T A); (8623 11130317 TA T); (8624 11130459 C A); (8625 11130473 T
C); (8626 11130687 A G); (8627 11132024 TTTC T); (8628 11132028 T C); (8629 11132076 A G); (8630
11132093 G A); (8631 11132131 A G); (8632 11132143 C T); (8633 11132204 G A); (8634 11132225 G
A); (8635 11132814 T C); (8636 11133003 A G); (8637 11133039 T A); (8638 11133087 G A); (8639
11133097 A T); (8640 11133180 A G); (8641 11133286 G A); (8642 11133358 G
GGTTCTGGGCGTGACCAGTCATGCCCA); (8643 11133558 G A); (8644 11133654 T A); (8645 11133782 C
T); (8646 11133825 C T); (8647 11134058 A C); (8648 11134317 G A); (8649 11137543 T TA); (8650
11137599 T C); (8651 11137675 TA T); (8652 11138353 A T); (8653 11138354 A T); (8654 11138847 A
G); (8655 11139091 TA T); (8656 11139536 G A); (8657 11140109 A AT); (8658 11141067 T C); (8659
11141184 AT A); (8660 11141274 T G); (8661 11141557 A T); (8662 11141650 C A); (8663 11143173 A
G); (8664 11145012 T G); (8665 11145350 C T); (8666 11145535 C CA); (8667 11145573 A G); (8668
11145655 A G); (8669 11145801 G A); (8670 11145898 C A); (8671 11146173 C T); (8672 11146297 C
T); (8673 11146372 T C); (8674 11146443 G T); (8675 11146447 G A); (8676 11146517 T C); (8677
11146598 C G); (8678 11146643 C A); (8679 11146719 A T); (8680 11146812 C T); (8681 11146821 C
A); (8682 11146828 G C); (8683 11146837 A C); (8684 11146873 T A); (8685 11146891 T C); (8686
11147195 A T); (8687 11147319 A G); (8688 11147402 G C); (8689 11147487 T C); (8690 11147713 C
T); (8691 11147825 T A); (8692 11147837 A T); (8693 11147844 AATAC A); (8694 11147848 A T); (8695
11147936 T G); (8696 11147946 A C); (8697 11147968 T C); (8698 11148013 G C); (8699 11148105 T
C); (8700 11148212 C CTA); (8701 11148394 C T); (8702 11148416 C T); (8703 11148454 T A); (8704
11148692 C T); (8705 11148707 G T); (8706 11148713 A G); (8707 11148740 A G); (8708 11148780 G
C); (8709 11148785 A T); (8710 11148789 A G); (8711 11148839 C A); (8712 11148853 A G); (8713
11148880 T G); (8714 11149005 GA G); (8715 11149023 A G); (8716 11149228 C T); (8717 11149324 A
G); (8718 11149435 A T); (8719 11149472 T C); (8720 11149563 G A); (8721 11149564 G T); (8722
11149634 A ATAAAT); (8723 11149724 T TCATG); (8724 11149807 G A); (8725 11149853 A T); (8726
11149981 A G); (8727 11150069 C A); (8728 11150082 C T); (8729 11150134 G A); (8730 11150142 A
ATCT); (8731 11150220 A C); (8732 11150517 G A); (8733 11150552 CAT C); (8734 11150771 G C); (8735
11150939 A C); (8736 11151016 A T); (8737 11151208 T A); (8738 11151332 G A); (8739 11151369 T
C); (8740 11151593 C A); (8741 11151594 A C); (8742 11151667 T TTTTAATAATA); (8743 11151845 C
T); (8744 11151857 A AT); (8745 11151953 A T); (8746 11152013 C T); (8747 11152017 A G); (8748
11152020 C A); (8749 11152089 A AT); (8750 11152138 A G); (8751 11152149 T C); (8752 11152197 G
A); (8753 11153178 T A); (8754 11153255 T A); (8755 11153406 C T); (8756 11153624 T TA); (8757
11153886 C T); (8758 11153911 C T); (8759 11153925 G T); (8760 11153950 C A); (8761 11154009 T
C); (8762 11154012 A T); (8763 11154078 C G); (8764 11154182 A T); (8765 11154362 CA C); (8766
11154603 C G); (8767 11154670 C G); (8768 11154791 G A); (8769 11154849 T TA); (8770 11154881 C
T); (8771 11154941 A C); (8772 11154988 C T); (8773 11155153 C A); (8774 11155231 C T); (8775
11155282 C T); (8776 11155292 G GA); (8777 11155556 GTCTT G); (8778 11155568 G A); (8779
11155571 G A); (8780 11155576 A C); (8781 11155582 T G); (8782 11155643 A G); (8783 11155719 G
T); (8784 11155736 T A); (8785 11155746 T C); (8786 11155752 T C); (8787 11155794 T TTA); (8788
11155804 T C); (8789 11155883 C T); (8790 11155930 T G); (8791 11156053 T A); (8792 11156054 T
C); (8793 11156058 A T); (8794 11156067 T C); (8795 11156231 C T); (8796 11156273 T C); (8797
11156311 A T); (8798 11156456 G A); (8799 11156600 G T); (8800 11156748 G A); (8801 11156903 T
C); (8802 11156939 G A); (8803 11159855 G A); (8804 11165309 C T); (8805 11165343 C T); (8806
11166336 C A); (8807 11168140 T C); (8808 11168169 G A); (8809 11168342 T G); (8810 11168574 T
A); (8811 11168736 A T); (8812 11168801 A ATT); (8813 11168940 C T); (8814 11168985 C T); (8815
11169073 A G); (8816 11169199 A G); (8817 11169253 G A); (8818 11169265 T G); (8819 11169521 G
A); (8820 11169558 T C); (8821 11169602 C A); (8822 11169671 T C); (8823 11169791 G A); (8824
11169795 T A); (8825 11170001 G A); (8826 11170075 C T); (8827 11170501 T A); (8828 11170516 T
C); (8829 11170520 A G); (8830 11170805 T G); (8831 11170847 C T); (8832 11171094 G A); (8833
11171124 T C); (8834 11171143 T C); (8835 11171315 C T); (8836 11171364 C T); (8837 11171396 T
C); (8838 11171419 G A); (8839 11171424 T C); (8840 11171439 G A); (8841 11171440 T C); (8842
11171441 G A); (8843 11171452 C T); (8844 11171515 T A); (8845 11171520 G
GCCACACCCCACGCGACCCGCA); (8846 11171528 T C); (8847 11171563 T C); (8848 11171571 G A); (8849
11171573 G A); (8850 11171880 G A); (8851 11171899 A G); (8852 11171999 C T); (8853 11172063 C
T); (8854 11172126 T C); (8855 11172217 C T); (8856 11172248 A G); (8857 11172266 T C); (8858
11172280 T C); (8859 11172340 A C); (8860 11172412 G A); (8861 11172522 G T); (8862 11172582 T
C); (8863 11172625 A G); (8864 11172886 G A); (8865 11172893 T C); (8866 11172922 C T); (8867
11172923 A G); (8868 11172925 C T); (8869 11173041 A G); (8870 11173064 G A); (8871 11173128 A
G); (8872 11173238 T C); (8873 11173249 A G); (8874 11173321 G A); (8875 11173368 A G); (8876
11173382 G A); (8877 11173390 C T); (8878 11173407 A G); (8879 11173417 C T); (8880 11173451 G
A); (8881 11173610 A C); (8882 11173632 C A); (8883 11173708 G A); (8884 11173712 A C); (8885
11173737 A T); (8886 11173757 G A); (8887 11173760 T C); (8888 11173774 T C); (8889 11173794 G
A); (8890 11173892 C T); (8891 11173929 C T); (8892 11173997 AC A); (8893 11174060 T C); (8894
11174123 C T); (8895 11174208 C T); (8896 11174215 T C); (8897 11174230 T G); (8898 11174265 C
T); (8899 11174324 A T); (8900 11174393 A G); (8901 11174396 T C); (8902 11174433 A G); (8903
11174486 C T); (8904 11174493 G A); (8905 11174500 G A); (8906 11174526 G A); (8907 11174549 T
C); (8908 11174568 A C); (8909 11174585 C T); (8910 11174587 G C); (8911 11174640 G C); (8912
11174900 C A); (8913 11175056 T TAA); (8914 11175346 G T); (8915 11175366 T G); (8916 11175708 T
A); (8917 11175716 C T); (8918 11175980 CTTGATAA C); (8919 11176082 T TA); (8920 11176122 C
T); (8921 11176139 T C); (8922 11176177 A G); (8923 11176198 T C); (8924 11176221 G T); (8925
11176229 T C); (8926 11176235 G A); (8927 11176376 T C); (8928 11176381 C T); (8929 11176426 T
C); (8930 11176451 TA T); (8931 11176724 A G); (8932 11176727 C T); (8933 11176775 G A); (8934
11176795 A C); (8935 11176820 A T); (8936 11176868 G T); (8937 11176983 AT A); (8938 11179975 G
A); (8939 11182427 G C); (8940 11184301 C G); (8941 11185723 G GA); (8942 11187456 C T); (8943
11187731 T C); (8944 11187885 A G); (8945 11189940 G A); (8946 11190480 G A); (8947 11190913 G
T); (8948 11191395 AACCCTAGCCAAACCCTAAACCCAAG A); (8949 11192205 A G); (8950 11192623 T
C); (8951 11193267 G T); (8952 11194149 T C); (8953 11194267 A G); (8954 11196000 T C); (8955
11199054 C T); (8956 11199997 C T); (8957 11200230 C T); (8958 11204201 C G); (8959 11204316 C
G); (8960 11205269 C A); (8961 11205960 C T); (8962 11206012 T C); (8963 11206255 C A); (8964
11206969 T C); (8965 11207702 C T); (8966 11208285 C T); (8967 11208792 C A); (8968 11210245 C
T); (8969 11213151 G T); (8970 11213923 T G); (8971 11214045 C A); (8972 11214224 A G); (8973
11214613 G A); (8974 11220652 G A); (8975 11222229 CT C); (8976 11223236 A T); (8977 11232861 C
T); (8978 11233265 ATTTG A); (8979 11235140 T G); (8980 11237623 G A); (8981 11237999 C
CTGTATATTCGTTTGTT); (8982 11238382 A AGAATT); (8983 11238602 C T); (8984 11240062 C
CAATT); (8985 11244847 G A); (8986 11245425 G A); (8987 11246436 G A); (8988 11248668 G A); (8989
11250396 C T); (8990 11259444 A G); (8991 11259504 C A); (8992 11259746 C T); (8993 11262160 G
A); (8994 11262504 C T); (8995 11263748 G C); (8996 11264607 G C); (8997 11264723 G A); (8998
11265269 C T); (8999 11266531 A G); (9000 11268476 CT C); (9001 11268740 G A); (9002 11270024 C
T); (9003 11272869 C T); (9004 11273636 G T); (9005 11275077 T A); (9006 11275996 A T); (9007
11276441 C T); (9008 11276682 A G); (9009 11276972 C T); (9010 11284233 G C); (9011 11285211 C
G); (9012 11292810 T C); (9013 11294172 C T); (9014 11296445 G A); (9015 11298325 ATT A); (9016
11300916 C A); (9017 11304912 A C); (9018 11308169 G A); (9019 11309821 AT A); (9020 11310319 C
T); (9021 11313483 C T); (9022 11313631 C T); (9023 11314276 G T); (9024 11315017 G A); (9025
11317267 G A); (9026 11317499 G A); (9027 11317523 T C); (9028 11317551 T
TACACACACACACACACACACACACACACAC); (9029 11320442 A T); (9030 11326616 G A); (9031
11327699 G A); (9032 11334471 C T); (9033 11334553 G A); (9034 11337078 C T); (9035 11337081 G
A); (9036 11338851 T C); (9037 11338857 C A); (9038 11338871 A C); (9039 11338880 A C); (9040
11338885 A T); (9041 11338897 C CAGAAAGCCAACTGGTAGGTACTGTTGT); (9042 11344519 C T); (9043
11349758 G A); (9044 11349981 A C); (9045 11350811 G A); (9046 11352618 G GT); (9047 11353236 C
T); (9048 11356557 G A); (9049 11358294 C T); (9050 11359657 G A); (9051 11359964 C T); (9052
11360321 C T); (9053 11365604 C A); (9054 11366044 A C); (9055 11366146 C T); (9056 11366665 T
A); (9057 11367278 G A); (9058 11373930 T G); (9059 11375730 T A); (9060 11377911 GT G); (9061
11382090 A G); (9062 11382733 C T); (9063 11383132 C T); (9064 11384237 A C); (9065 11385169 A
G); (9066 11386745 T C); (9067 11387952 T C); (9068 11388076 A T); (9069 11388762 G C); (9070
11390287 C T); (9071 11390450 C CT); (9072 11391526 T C); (9073 11393111 G A); (9074 11395064 T
G); (9075 11395102 A G); (9076 11395350 A G); (9077 11395943 G A); (9078 11396356 T C); (9079
11397375 T G); (9080 11397681 G A); (9081 11397964 T G); (9082 11398374 C T); (9083 11398544 G
A); (9084 11398617 C T); (9085 11399062 G T); (9086 11399482 T C); (9087 11400424 T A); (9088
11400735 A G); (9089 11401048 A C); (9090 11401654 A G); (9091 11401763 C G); (9092 11403044 A
G); (9093 11403394 A G); (9094 11405499 G A); (9095 11407301 G A); (9096 11408561 C T); (9097
11409203 G A); (9098 11409432 A C); (9099 11410038 C T); (9100 11410756 C G); (9101 11411203 T
C); (9102 11411595 A G); (9103 11411824 A G); (9104 11412602 A C); (9105 11412623
GACTCTTGTTCCCAAACCTAA G); (9106 11413088 T C); (9107 11415387 A G); (9108 11415980 G A); (9109
11416004 GT G); (9110 11416627 AT A); (9111 11418702 T C); (9112 11418901 G A); (9113 11419488 T
C); (9114 11421048 G A); (9115 11421713 C T); (9116 11421970 C G); (9117 11422046 G C); (9118
11427270 T C); (9119 11430287 T C); (9120 11431446 C A); (9121 11434473 C T); (9122 11436127 A
G); (9123 11436183 C CT); (9124 11436931 A T); (9125 11436934 G GT); (9126 11438200 A C); (9127
11439913 C T); (9128 11440881 T C); (9129 11445019 A G); (9130 11446414 G A); (9131 11447065 C
T); (9132 11449675 A C); (9133 11450464 C T); (9134 11451149 G A); (9135 11451834 G A); (9136
11452446 T C); (9137 11456001 ATTT A); (9138 11456733 G A); (9139 11458682 G A); (9140 11458940 G
C); (9141 11459012 C G); (9142 11459385 C T); (9143 11461456 T C); (9144 11461464 G A); (9145
11461621 CA C); (9146 11462161 A C); (9147 11462713 G C); (9148 11462743 C A); (9149 11462761 C
T); (9150 11463273 G A); (9151 11465948 G A); (9152 11468213 A G); (9153 11468616 C T); (9154
11468761 C T); (9155 11469344 C T); (9156 11470514 T C); (9157 11472501 T C); (9158 11472571 G
A); (9159 11473346 T C); (9160 11474634 A G); (9161 11475022 C T); (9162 11475914 A C); (9163
11476005 G T); (9164 11476419 G A); (9165 11477006 T A); (9166 11477811 T C); (9167 11477851 G
A); (9168 11478214 G A); (9169 11480220 A C); (9170 11481113 AGTTAT A); (9171 11484242 A C); (9172
11485290 A G); (9173 11485836 A T); (9174 11485996 A G); (9175 11487159 C T); (9176 11491528 G
A); (9177 11491950 A T); (9178 11492473 G A); (9179 11494622 C A); (9180 11495213 G C); (9181
11495762 T C); (9182 11496504 G A); (9183 11497739 G A); (9184 11502322 T A); (9185 11502332 G
A); (9186 11502363 C T); (9187 11502490 G A); (9188 11502597 A G); (9189 11508006 C T); (9190
11511062 T TACAACA); (9191 11511305 C A); (9192 11511310 C A); (9193 11512366 T A); (9194
11513744 A C); (9195 11514532 G C); (9196 11514931 T C); (9197 11516365 C T); (9198 11517130 C
T); (9199 11517709 C G); (9200 11518236 C T); (9201 11518391 T C); (9202 11519922 G A); (9203
11519949 G A); (9204 11519988 G A); (9205 11520201 A G); (9206 11520511 T C); (9207 11520584 C
T); (9208 11520622 C T); (9209 11522522 A G); (9210 11523131 G A); (9211 11523181 C A); (9212
11524149 C T); (9213 11524612 G A); (9214 11524730 C T); (9215 11525304 G A); (9216 11525437 C
T); (9217 11525635 C T); (9218 11525797 A G); (9219 11526549 A G); (9220 11526856 C A); (9221
11527556 C A); (9222 11527680 G A); (9223 11527856 C T); (9224 11528229 T C); (9225 11528259 T
C); (9226 11530067 GT G); (9227 11530107 T C); (9228 11534016 T G); (9229 11536596 TA T); (9230
11538608 G A); (9231 11541271 A G); (9232 11542888 TA T); (9233 11543667 C G); (9234 11544531 C
G); (9235 11545884 T C); (9236 11546074 G C); (9237 11549694 C T); (9238 11549896 C A); (9239
11550082 G A); (9240 11550920 C T); (9241 11551265 C T); (9242 11552052 C T); (9243 11552781 G
T); (9244 11552877 G T); (9245 11552908 C T); (9246 11553311 T C); (9247 11554007 C T); (9248
11554066 C T); (9249 11561263 C T); (9250 11562622 T C); (9251 11567081 G A); (9252 11567209 C
T); (9253 11567635 GC G); (9254 11568082 G A); (9255 11568600 G A); (9256 11568858 C G); (9257
11569054 G A); (9258 11570697 C T); (9259 11573565 C T); (9260 11574556 T C); (9261 11576001 C
T); (9262 11577167 G C); (9263 11578518 G A); (9264 11578762 C T); (9265 11579396 C T); (9266
11579821 T C); (9267 11580900 C T); (9268 11582112 C T); (9269 11585407 G A); (9270 11586054 T
C); (9271 11587470 A G); (9272 11589007 A G); (9273 11589839 T G); (9274 11589981 G A); (9275
11590961 T C); (9276 11590983 C T); (9277 11593127 G C); (9278 11597777 C T); (9279 11598179 A
G); (9280 11598224 C T); (9281 11598773 T G); (9282 11598796 C T); (9283 11598828 A G); (9284
11598926 T C); (9285 11598929 G A); (9286 11598945 A C); (9287 11598981 CCAG C); (9288 11599987 T
C); (9289 11600050 A G); (9290 11600177 G A); (9291 11600341 G A); (9292 11602805 G C); (9293
11602983 T C); (9294 11602985 A G); (9295 11604060 GC G); (9296 11604123 A C); (9297 11604127 G
A); (9298 11604218 TCTCTCTTCC T); (9299 11604399 A G); (9300 11604696 A G); (9301 11604747 C
T); (9302 11604749 T C); (9303 11604762 A T); (9304 11606561 G A); (9305 11609808 C T); (9306
11610300 C T); (9307 11610964 C T); (9308 11614303 T G); (9309 11615339 CA C); (9310 11615932
TTTGGTACTGGA T); (9311 11616028 G A); (9312 11620381 C T); (9313 11621400 A G); (9314 11622176 C
T); (9315 11623132 A G); (9316 11623469 G A); (9317 11623588 A G); (9318 11623613 T C); (9319
11623687 C T); (9320 11623703 T C); (9321 11624735 GT G); (9322 11624823 G A); (9323 11625018 G
A); (9324 11625032 T G); (9325 11625901 C T); (9326 11625914 A G); (9327 11626313 C T); (9328
11626459 T C); (9329 11626763 T C); (9330 11626808 G T); (9331 11627475 C T); (9332 11628798 C
G); (9333 11629519 A C); (9334 11629543 A G); (9335 11629676 A G); (9336 11629766 A G); (9337
11629771 A G); (9338 11630102 C G); (9339 11630148 A G); (9340 11630295 C T); (9341 11630321 T
C); (9342 11630385 T C); (9343 11630446 T C); (9344 11630465 A G); (9345 11630536 G T); (9346
11630744 T A); (9347 11631000 C A); (9348 11631539 C T); (9349 11632047 G A); (9350 11632183 T
G); (9351 11632399 A G); (9352 11632443 C A); (9353 11632730 T C); (9354 11632778 T C); (9355
11633022 G A); (9356 11633068 C G); (9357 11633151 C T); (9358 11633333 T C); (9359 11633381 T
C); (9360 11633403 T C); (9361 11633467 T C); (9362 11633645 T G); (9363 11634520 G A); (9364
11634540 T C); (9365 11634630 C T); (9366 11635220 G A); (9367 11635262 T C); (9368 11636264 A
G); (9369 11636485 G A); (9370 11636696 C A); (9371 11636823 C T); (9372 11636847 C T); (9373
11636895 G T); (9374 11636973 T C); (9375 11637063 C A); (9376 11638076 C T); (9377 11638141 T
C); (9378 11638176 A T); (9379 11638195 A G); (9380 11638269 C T); (9381 11638371 G A); (9382
11638393 A T); (9383 11638459 A G); (9384 11638562 C A); (9385 11638662 T C); (9386 11638702 A
C); (9387 11638873 G C); (9388 11638884 C T); (9389 11638927 G A); (9390 11638985 A G); (9391
11638994 C T); (9392 11639051 A T); (9393 11639140 C T); (9394 11639161 T C); (9395 11639164 C
T); (9396 11639178 C T); (9397 11639181 C T); (9398 11639196 T A); (9399 11639202 A G); (9400
11639222 A G); (9401 11639225 G T); (9402 11639261 T C); (9403 11639266 A C); (9404 11639268 T
A); (9405 11641804 A G); (9406 11641812 T G); (9407 11641831 C T); (9408 11641835 G A); (9409
11641841 G T); (9410 11641891 A G); (9411 11641930 C T); (9412 11642048 T A); (9413 11642070 T
C); (9414 11642105 G A); (9415 11642110 A G); (9416 11642118 C T); (9417 11642140 G A); (9418
11642144 A G); (9419 11642163 T C); (9420 11642165 T A); (9421 11642204 A C); (9422 11642241 C
T); (9423 11642255 T G); (9424 11642353 TG T); (9425 11642470 G A); (9426 11642519 C T); (9427
11642916 C T); (9428 11642918 C A); (9429 11642920 T A); (9430 11642939 G A); (9431 11642947 TC
T); (9432 11642948 C T); (9433 11643009 C T); (9434 11643055 G A); (9435 11643095 T C); (9436
11643097 TA T); (9437 11643099 C T); (9438 11643530 A T); (9439 11643841 C A); (9440 11643851 C
T); (9441 11643859 T A); (9442 11643866 T C); (9443 11643986 C T); (9444 11644028 T C); (9445
11644296 T C); (9446 11644299 C A); (9447 11644341 T G); (9448 11644433 C T); (9449 11644491 A
C); (9450 11644505 A G); (9451 11644531 C CT); (9452 11644680 G A); (9453 11644692 C A); (9454
11644720 C A); (9455 11644723 T C); (9456 11644841 G A); (9457 11645052 A T); (9458 11645062 T
A); (9459 11645117 G C); (9460 11645128 C T); (9461 11645214 C T); (9462 11645229 A G); (9463
11645234 C A); (9464 11645235 T A); (9465 11645237 C T); (9466 11645243 A G); (9467 11645251 A
G); (9468 11645295 A T); (9469 11645315 C T); (9470 11645384 C T); (9471 11645405 T C); (9472
11645422 C T); (9473 11645450 G A); (9474 11645492 T C); (9475 11645497 A G); (9476 11645498 A
G); (9477 11645505 T C); (9478 11645512 T C); (9479 11645761 A G); (9480 11645775 G GT); (9481
11645785 A C); (9482 11645796 C T); (9483 11645872 A G); (9484 11645990 A G); (9485 11646094 A
G); (9486 11646124 T A); (9487 11646128 C CT); (9488 11646134 A G); (9489 11646138 A G); (9490
11646751 A C); (9491 11646757 C T); (9492 11646817 G A); (9493 11646825 T C); (9494 11646839 G
A); (9495 11648075 C T); (9496 11648316 G A); (9497 11648474 G C); (9498 11648663 G A); (9499
11648854 A G); (9500 11649213 C A); (9501 11649509 C T); (9502 11650581 G T); (9503 11650583 C
CTT); (9504 11650940 G A); (9505 11650956 G A); (9506 11651098 A G); (9507 11651245 T C); (9508
11651816 T G); (9509 11652054 C T); (9510 11652129 G A); (9511 11652168 G A); (9512 11652246 A
G); (9513 11654007 T G); (9514 11654442 A G); (9515 11655996 G A); (9516 11656819 C A); (9517
11656850 G A); (9518 11657070 T A); (9519 11659153 GTAA G); (9520 11659166 G C); (9521 11659284
C T); (9522 11659295 T C); (9523 11659401 A C); (9524 11659573 A G); (9525 11659884 G A); (9526
11659981 T C); (9527 11660125 T TA); (9528 11660160 G A); (9529 11660346 G A); (9530 11660526 G
A); (9531 11660634 C A); (9532 11660707 A C); (9533 11660709 A T); (9534 11660711 T C); (9535
11660937 T G); (9536 11661102 G T); (9537 11661184 A G); (9538 11662329 T TA); (9539 11663370 A
G); (9540 11663630 C T); (9541 11664378 T G); (9542 11664448 T C); (9543 11664792 A C); (9544
11664797 C G); (9545 11664802 C G); (9546 11665853 T A); (9547 11665960 T C); (9548 11665976 A
C); (9549 11666017 A T); (9550 11666137 G A); (9551 11666699 C G); (9552 11666832 T C); (9553
11666856 GA G); (9554 11666946 A G); (9555 11666950 C G); (9556 11667153 G GA); (9557 11667358 T
A); (9558 11667567 C T); (9559 11667898 G A); (9560 11668395 C T); (9561 11668583 C T); (9562
11668851 G C); (9563 11669657 C T); (9564 11669715 C T); (9565 11669766 A AC); (9566 11670061 G
A); (9567 11670130 A G); (9568 11670141 G A); (9569 11670176 A G); (9570 11670284 C T); (9571
11670356 G A); (9572 11670368 C A); (9573 11670411 A T); (9574 11670655 A G); (9575 11670859 G
A); (9576 11670879 A G); (9577 11670934 T C); (9578 11670999 C A); (9579 11671139 T A); (9580
11671631 C T); (9581 11672014 A G); (9582 11672734 T C); (9583 11672785 T C); (9584 11672887 A
G); (9585 11674169 C T); (9586 11674193 G A); (9587 11674275 C G); (9588 11674370 T C); (9589
11674397 A G); (9590 11674439 C T); (9591 11674462 G A); (9592 11674790 G A); (9593 11674795 T
G); (9594 11674917 T C); (9595 11674997 T C); (9596 11675067 T C); (9597 11675275 C T); (9598
11675289 C CGTGGGAGCCCACGCTTGGAGT); (9599 11675291 G GGCGTGGGAGCCCACGCTTGGA); (9600
11675303 A G); (9601 11675511 C T); (9602 11675686 T C); (9603 11675729 T C); (9604 11675802 C
T); (9605 11675888 A C); (9606 11675921 G A); (9607 11675965 A G); (9608 11675970 GC G); (9609
11676103 T C); (9610 11676118 G A); (9611 11676534 A C); (9612 11676838 T G); (9613 11676877 A
G); (9614 11677106 C T); (9615 11677146 T C); (9616 11677592 A G); (9617 11677618
TAAAACCTTGTTTATC T); (9618 11677628 G A); (9619 11677743 G A); (9620 11677744 C T); (9621
11677949 T G); (9622 11678093 C T); (9623 11678206 T G); (9624 11678412 G A); (9625 11678421 T
C); (9626 11678430 C T); (9627 11678528 AGTT A); (9628 11678745 A G); (9629 11678787 A G); (9630
11678840 T C); (9631 11678894 T G); (9632 11678903 G A); (9633 11679053 C T); (9634 11679137 T
A); (9635 11679174 C T); (9636 11679567 T G); (9637 11679865 C T); (9638 11679875 A G); (9639
11680033 T A); (9640 11680137 A G); (9641 11680231 G A); (9642 11680241 G A); (9643 11680392 C
T); (9644 11680467 A G); (9645 11680504 G A); (9646 11680625 G A); (9647 11680814 G C); (9648
11680933 C T); (9649 11680964 A C); (9650 11681198 T C); (9651 11681222 A AT); (9652 11681298 G
A); (9653 11681968 G A); (9654 11682012 C T); (9655 11682058 C A); (9656 11682141 T G); (9657
11682383 A G); (9658 11682460 C A); (9659 11682631 A ATACGT); (9660 11682755 G T); (9661
11683012 C T); (9662 11683762 C T); (9663 11684349 C T); (9664 11684389 G A); (9665 11684570 G
A); (9666 11684861 C T); (9667 11685079 A C); (9668 11685125 T C); (9669 11685315 G A); (9670
11685422 A G); (9671 11685845 T A); (9672 11685861 C A); (9673 11685881 T C); (9674 11685891 G
A); (9675 11685981 T C); (9676 11685993 G C); (9677 11686048 G A); (9678 11686253 T G); (9679
11686284 A G); (9680 11686369 T C); (9681 11686627 C T); (9682 11687136 T C); (9683 11687205 G
T); (9684 11687219 T A); (9685 11687388 C T); (9686 11687406 T TA); (9687 11687487 G A); (9688
11687611 T TG); (9689 11687739 T G); (9690 11687837 T C); (9691 11687919 G A); (9692 11688006 T
C); (9693 11688880 T C); (9694 11689220 C A); (9695 11689274 T C); (9696 11689482 G C); (9697
11689708 A C); (9698 11689848 G A); (9699 11689944 G C); (9700 11690314 T G); (9701 11690422 A
G); (9702 11690638 C T); (9703 11690837 A T); (9704 11690933 T A); (9705 11691166 C A); (9706
11691394 A T); (9707 11691459 C A); (9708 11691660 G A); (9709 11691897 G A); (9710 11691936 C
T); (9711 11691979 A G); (9712 11693318 T A); (9713 11693326 C T); (9714 11693380 C A); (9715
11693405 C A); (9716 11693506 A G); (9717 11693740 C T); (9718 11693748 G T); (9719 11694299 T
C); (9720 11694321 A T); (9721 11694409 T G); (9722 11694466 AT A); (9723 11694507 A C); (9724
11694554 T C); (9725 11694562 T TGATGGCGCC); (9726 11694615 A C); (9727 11694667 G A); (9728
11694691 G T); (9729 11694848 A G); (9730 11694955 T C); (9731 11694983 A G); (9732 11695149 C
T); (9733 11695155 C T); (9734 11695621 G A); (9735 11695713 T C); (9736 11696008 A G); (9737
11696283 C T); (9738 11696317 C T); (9739 11696318 A G); (9740 11696391 T A); (9741 11696455 T
C); (9742 11696509 C T); (9743 11696818 G A); (9744 11696925 A C); (9745 11697555 A G); (9746
11697572 G A); (9747 11697631 C G); (9748 11697728 C A); (9749 11697836 T C); (9750 11697876 A
G); (9751 11698371 A T); (9752 11698709 T C); (9753 11698861 T C); (9754 11698995 A G); (9755
11699469 T A); (9756 11699476 A C); (9757 11699714 G C); (9758 11700099 T A); (9759 11700132 G
A); (9760 11700174 G A); (9761 11700415 A C); (9762 11700504 C T); (9763 11700520 C T); (9764
11700723 T C); (9765 11700815 C G); (9766 11700892 A C); (9767 11700991 C A); (9768 11701112 G
A); (9769 11701518 G T); (9770 11701962 G A); (9771 11702186 A C); (9772 11702298 C T); (9773
11702426 G A); (9774 11702615 T C); (9775 11702757 T C); (9776 11702758 G A); (9777 11702762 A
C); (9778 11702799 T C); (9779 11702847 A AC); (9780 11702853 A T); (9781 11702914 A G); (9782
11703409 A G); (9783 11703544 A G); (9784 11703653 G T); (9785 11703689 T G); (9786 11703971 A
G); (9787 11704070 C G); (9788 11704112 G A); (9789 11704532 T C); (9790 11704740 G A); (9791
11704789 G A); (9792 11704856 C T); (9793 11705003 A G); (9794 11705299 G A); (9795 11705530 C
T); (9796 11705567 G A); (9797 11705693 A C); (9798 11705960 A G); (9799 11706081 C T); (9800
11706126 T C); (9801 11706683 CT C); (9802 11706918 G A); (9803 11707110 T C); (9804 11707119 G
A); (9805 11707139 T A); (9806 11707390 T TA); (9807 11707501 G A); (9808 11707582 T C); (9809
11707774 A T); (9810 11707827 T C); (9811 11708050 C T); (9812 11708113 G A); (9813 11708921 A
C); (9814 11709049 T G); (9815 11709310 G A); (9816 11709334 C T); (9817 11709497 A G); (9818
11709550 A G); (9819 11709562 T C); (9820 11709648 G C); (9821 11710373 C T); (9822 11710384 T
C); (9823 11710395 G GA); (9824 11710450 G A); (9825 11710950 A G); (9826 11711321 A G); (9827
11711695 A G); (9828 11711706 A G); (9829 11711777 T TA); (9830 11711786 A T); (9831 11711859 G
A); (9832 11712940 A G); (9833 11713049 C T); (9834 11713114 T C); (9835 11713137 A C); (9836
11713461 C A); (9837 11713881 A G); (9838 11714530 A G); (9839 11714665 T C); (9840 11714676 A
G); (9841 11715074 G A); (9842 11716291 G A); (9843 11716722 T C); (9844 11716813 A G); (9845
11717295 TACATAC T); (9846 11717351 G A); (9847 11717594 A G); (9848 11717742 T C); (9849
11718097 T A); (9850 11718098 T C); (9851 11718449 G A); (9852 11718587 T C); (9853 11718685 A
G); (9854 11718793 A G); (9855 11719143 C A); (9856 11719223 A T); (9857 11719426 G A); (9858
11719725 C T); (9859 11720039 T C); (9860 11720385 C T); (9861 11720393 A C); (9862 11720400 A
G); (9863 11720405 T A); (9864 11720421 G A); (9865 11720426 A G); (9866 11720440 C A); (9867
11720443 A T); (9868 11720742 T A); (9869 11720994 A G); (9870 11721038 G C); (9871 11721090 T
C); (9872 11721097 C T); (9873 11721157 A G); (9874 11721478 T C); (9875 11721480 G A); (9876
11722085 C T); (9877 11722450 TA T); (9878 11722685 A AT); (9879 11722733 A G); (9880 11722973 T
C); (9881 11723190 G GA); (9882 11723305 AT A); (9883 11723413 T TA); (9884 11723552 T A); (9885
11723955 T G); (9886 11724337 T A); (9887 11724513 C T); (9888 11724626 C A); (9889 11724752 G
T); (9890 11724775 A G); (9891 11724957 G A); (9892 11725003 G A); (9893 11725005 A G); (9894
11725007 G A); (9895 11725089 A G); (9896 11725106 G A); (9897 11725116 A C); (9898 11725151 C
T); (9899 11725254 A G); (9900 11725303 C T); (9901 11725331 A G); (9902 11725376 G A); (9903
11725837 A G); (9904 11726075 G A); (9905 11726138 C T); (9906 11726334 G A); (9907 11726450 G
A); (9908 11726532 C T); (9909 11726937 A G); (9910 11727255 A G); (9911 11727296 C A); (9912
11727317 G A); (9913 11727438 T C); (9914 11727841 C T); (9915 11727852 A C); (9916 11728152 G
A); (9917 11728419 G GT); (9918 11728743 T C); (9919 11728802 C G); (9920 11729228 C
CTAGGGTT); (9921 11729395 A AGTTTAGGGTTTGG); (9922 11729936 G A); (9923 11729939 G A); (9924
11730011 T G); (9925 11730019 A G); (9926 11730057 A G); (9927 11730082 A G); (9928 11730242 T
G); (9929 11730302 G C); (9930 11730444 C A); (9931 11730585 T C); (9932 11730733 A G); (9933
11730863 T TA); (9934 11730912 C T); (9935 11732397 C A); (9936 11732528 G T); (9937 11733442 G
A); (9938 11734841 G A); (9939 11735173 C T); (9940 11735562 A G); (9941 11735599 A G); (9942
11735908 A G); (9943 11735934 A C); (9944 11735955 G A); (9945 11736203 C A); (9946 11736388 T
C); (9947 11736402 A T); (9948 11736747 T C); (9949 11736753 C T); (9950 11736758 C G); (9951
11736861 T G); (9952 11737500 C T); (9953 11737591 T C); (9954 11737772 C A); (9955 11737825 A
C); (9956 11738056 T C); (9957 11738066 A G); (9958 11738277 G C); (9959 11738367 C T); (9960
11738497 A G); (9961 11738923 C T); (9962 11739275 G A); (9963 11739280 G GAA); (9964 11739491 C
T); (9965 11739567 G A); (9966 11739641 T C); (9967 11739690 A G); (9968 11739731 G A); (9969
11739830 G A); (9970 11739847 T C); (9971 11739909 A C); (9972 11739999 C T); (9973 11740058 G
A); (9974 11740096 C T); (9975 11740377 A C); (9976 11740506 T C); (9977 11740758 T C); (9978
11740868 A C); (9979 11741011 G A); (9980 11741057 G A); (9981 11741203 G A); (9982 11741581 C
T); (9983 11741952 T A); (9984 11742099 A G); (9985 11743027 A G); (9986 11743180 G A); (9987
11743260 A T); (9988 11743276 A AG); (9989 11743336 A G); (9990 11743411 T G); (9991 11743928 C
T); (9992 11744496 A AAC); (9993 11744724 A G); (9994 11744939 T C); (9995 11744941 A T); (9996
11745361 G A); (9997 11745363 TG T); (9998 11745794 G A); (9999 11745837 A G); (10000 11745945 G
A); (10001 11746316 A G); (10002 11746342 C G); (10003 11746461 C A); (10004 11746482 A G); (10005
11746574 T C); (10006 11746701 C A); (10007 11746974 G T); (10008 11747030 A G); (10009 11747237
A G); (10010 11747270 T C); (10011 11747328 AAAAG A); (10012 11747337 A G); (10013 11747931 C
T); (10014 11748791 T A); (10015 11749006 GT G); (10016 11749533 T TTA); (10017 11749922
ATATATATATATATATAT A); (10018 11750787 T G); (10019 11751088 G A); (10020 11751178 G
A); (10021 11751561 T G); (10022 11753273 GA G); (10023 11754390 G T); (10024 11754485 G
A); (10025 11754779 T TA); (10026 11754905 C T); (10027 11755092 A C); (10028 11755834 T A); (10029
11756398 G A); (10030 11756434 A G); (10031 11756585 T G); (10032 11756714 C G); (10033 11756905
T C); (10034 11757262 T C); (10035 11757439 G A); (10036 11757467 C T); (10037 11757552 C G); (10038
11757635 G A); (10039 11757643 G A); (10040 11757764 C T); (10041 11758021 G A); (10042 11758228
A G); (10043 11758741 G A); (10044 11758796 A G); (10045 11758917 A G); (10046 11758927 C
T); (10047 11759081 G A); (10048 11759914 C A); (10049 11760665 G A); (10050 11763598 G A); (10051
11764485 T C); (10052 11764496 G A); (10053 11764509 C T); (10054 11764511 G A); (10055 11766717
C T); (10056 11766754 T C); (10057 11766804 A G); (10058 11766885 A G); (10059 11766982 G
A); (10060 11766998 G A); (10061 11767175 G A); (10062 11767484 T C); (10063 11767725 T A); (10064
11768439 C T); (10065 11768624 TG T); (10066 11768712 G A); (10067 11768724 G A); (10068
11768998 C T); (10069 11770286 A C); (10070 11770396 A C); (10071 11770557 T A); (10072 11771215
A T); (10073 11771471 C T); (10074 11771502 G C); (10075 11771788 T C); (10076 11772024 T C); (10077
11772413 T C); (10078 11772445 T C); (10079 11772481 G A); (10080 11772653 G T); (10081 11772917
T C); (10082 11773100 A G); (10083 11773210 A G); (10084 11773807 G A); (10085 11774127 G
A); (10086 11774166 C T); (10087 11774168 C A); (10088 11774223 A T); (10089 11774650 C A); (10090
11774818 A G); (10091 11775375 C T); (10092 11775384 C A); (10093 11775487 T C); (10094 11775508
G A); (10095 11776059 T C); (10096 11776520 G A); (10097 11776566 T C); (10098 11782802 T
C); (10099 11783450 A G); (10100 11789579 T C); (10101 11789765 C T); (10102 11789792 C G); (10103
11789866 T A); (10104 11789886 T A); (10105 11790101 G A); (10106 11790694 G T); (10107 11790729
G T); (10108 11791082 G A); (10109 11791114 T A); (10110 11792990 G A); (10111 11793134 C
T); (10112 11793137 A G); (10113 11793757 CA C); (10114 11794154 C T); (10115 11794187 G A); (10116
11794481 A G); (10117 11794494 A C); (10118 11794527 A T); (10119 11794575 T C); (10120 11794580
T C); (10121 11794656 C T); (10122 11795019 G A); (10123 11795048 T C); (10124 11795812 T C); (10125
11795849 G A); (10126 11795930 T C); (10127 11795939 A G); (10128 11796330 G C); (10129 11796402
G C); (10130 11796486 C T); (10131 11796513 C T); (10132 11796562 G A); (10133 11796659 G
A); (10134 11796697 T A); (10135 11796753 C T); (10136 11796793 G T); (10137 11796798 T C); (10138
11796874 T C); (10139 11797282 G C); (10140 11797297 T C); (10141 11797435 G A); (10142 11797851
G A); (10143 11798240 T C); (10144 11798373 C T); (10145 11798500 C A); (10146 11798520 T A); (10147
11798568 A G); (10148 11798618 C T); (10149 11798840 A T); (10150 11798852 C A); (10151 11798862
G T); (10152 11798868 T G); (10153 11798869 T A); (10154 11798870 T A); (10155 11798871 T A); (10156
11798888 G A); (10157 11798996 G A); (10158 11799038 C T); (10159 11799168 T C); (10160 11799175
T C); (10161 11799370 T C); (10162 11799391 G A); (10163 11799415 C G); (10164 11799478 G
T); (10165 11799865 C T); (10166 11799866 G T); (10167 11800180 TA T); (10168 11800275 G A); (10169
11800282 T C); (10170 11800404 T C); (10171 11800513 A G); (10172 11801071 G T); (10173 11801352
T C); (10174 11801388 C A); (10175 11801950 G C); (10176 11801977 G T); (10177 11802119 G
A); (10178 11802621 T A); (10179 11802677 A AAAAG); (10180 11802739 C T); (10181 11802778 C
T); (10182 11802785 T C); (10183 11803082 C T); (10184 11803098 A G); (10185 11803155 A G); (10186
11803223 A G); (10187 11803226 G A); (10188 11803254 C A); (10189 11803404 G A); (10190 11803433
G A); (10191 11803528 A G); (10192 11804033 A G); (10193 11804136 A G); (10194 11804261 G
C); (10195 11804427 G C); (10196 11804556 T C); (10197 11804579 G C); (10198 11804717 T G); (10199
11804870 A C); (10200 11805057 A G); (10201 11805270 C T); (10202 11805392 A T); (10203 11805722
AC A); (10204 11805932 G A); (10205 11806295 G A); (10206 11806520 A G); (10207 11806549 A
G); (10208 11806592 G A); (10209 11806624 A G); (10210 11807555 C T); (10211 11807734 A G); (10212
11807749 A G); (10213 11807961 A T); (10214 11808370 G A); (10215 11808447 C T); (10216 11808454
C T); (10217 11808825 G A); (10218 11809176 G T); (10219 11809329 T C); (10220 11809537 C
G); (10221 11810043 C CT); (10222 11810140 C T); (10223 11810153 A G); (10224 11810203 C T); (10225
11811005 A C); (10226 11811822 C T); (10227 11812121 T C); (10228 11812495 A G); (10229 11812727
T A); (10230 11812991 C A); (10231 11813141 A T); (10232 11813241 T TC); (10233 11813424 T
C); (10234 11813443 T A); (10235 11813467 C A); (10236 11813553 A G); (10237 11813771 A G); (10238
11813784 T C); (10239 11813997 C T); (10240 11814214 C CT); (10241 11814274 C T); (10242 11814543
T C); (10243 11814626 G A); (10244 11814735 C A); (10245 11814738 T C); (10246 11814897 A T); (10247
11814922 A C); (10248 11815084 T G); (10249 11815117 A G); (10250 11815169 G A); (10251 11815898
A G); (10252 11816074 A G); (10253 11816169 A C); (10254 11816285 GA G); (10255 11816965 C
T); (10256 11817229 C A); (10257 11817249 T C); (10258 11817335 T C); (10259 11817366 G A); (10260
11817719 A G); (10261 11817887 G T); (10262 11818304 A T); (10263 11818824 C T); (10264 11819324
T C); (10265 11825174 A G); (10266 11825310 C T); (10267 11828624 C T); (10268 11829356 A T); (10269
11829403 C T); (10270 11830367 T C); (10271 11830416 C A); (10272 11830474 T C); (10273 11830477 C
T); (10274 11830593 G T); (10275 11831336 T C); (10276 11831709 C T); (10277 11831884 T C); (10278
11832014 C T); (10279 11832149 G T); (10280 11832275 T A); (10281 11832337 T A); (10282 11832533 C
T); (10283 11832573 A T); (10284 11832936 T C); (10285 11833143 A G); (10286 11833341 T G); (10287
11833396 A G); (10288 11833585 A G); (10289 11833686 A G); (10290 11833690 T C); (10291 11833777
G A); (10292 11833837 C T); (10293 11834148 G A); (10294 11834190 G T); (10295 11834219 C
T); (10296 11834236 C T); (10297 11834403 A C); (10298 11834735 G A); (10299 11835085 A G); (10300
11835102 A G); (10301 11835284 G A); (10302 11835740 A G); (10303 11835830 T C); (10304 11835905
C T); (10305 11836029 T C); (10306 11836043 T C); (10307 11836046 C G); (10308 11836088 G A); (10309
11836305 G C); (10310 11836718 C A); (10311 11836764 T A); (10312 11837205 G T); (10313 11837244
A G); (10314 11837600 A G); (10315 11837736 C A); (10316 11837747 G A); (10317 11837991 A
G); (10318 11838110 A C); (10319 11838771 G T); (10320 11838891 C T); (10321 11838937 A G); (10322
11839115 C A); (10323 11839340 C A); (10324 11839566 C T); (10325 11839643 T G); (10326 11839875
C T); (10327 11839995 A C); (10328 11840013 A AAGCT); (10329 11840100 A G); (10330 11840249 A
G); (10331 11840467 T C); (10332 11840530 G T); (10333 11840775 A AT); (10334 11841329 C T); (10335
11841666 A G); (10336 11841957 G A); (10337 11842003 A G); (10338 11842136 A C); (10339 11842312
A G); (10340 11842485 T C); (10341 11842545 T C); (10342 11842768 G A); (10343 11842898 G
A); (10344 11842987 G A); (10345 11843767 A G); (10346 11844273 C T); (10347 11844385 C A); (10348
11844415 C CTAAT); (10349 11844772 A G); (10350 11844952 T A); (10351 11845048 GA G); (10352
11845286 T C); (10353 11845334 T C); (10354 11845344 C T); (10355 11846541 A AT); (10356 11846732
C A); (10357 11847102 G A); (10358 11847680 C T); (10359 11847749 T C); (10360 11847792 G
T); (10361 11847881 G A); (10362 11847941 C A); (10363 11848180 T C); (10364 11848218 A G); (10365
11848242 C A); (10366 11848259 G A); (10367 11848307 T G); (10368 11848531 T C); (10369 11848536
C T); (10370 11848913 A C); (10371 11849353 A G); (10372 11849554 A G); (10373 11849904 G
A); (10374 11850048 G T); (10375 11850078 T C); (10376 11850846 T C); (10377 11850862 G T); (10378
11851027 G A); (10379 11851070 C T); (10380 11851195 G C); (10381 11851294 C T); (10382 11851295
A T); (10383 11851985 A G); (10384 11852032 T C); (10385 11852154 A G); (10386 11852224 T
C); (10387 11852775 G A); (10388 11853115 T C); (10389 11853163 C T); (10390 11853257 T C); (10391
11853305 G A); (10392 11853325 G A); (10393 11853361 A T); (10394 11853363 AC A); (10395
11853438 G T); (10396 11853526 T C); (10397 11853848 C T); (10398 11853982 T G); (10399 11854226 T
C); (10400 11854417 G A); (10401 11854420 C T); (10402 11854486 A G); (10403 11854583 C T); (10404
11854711 T C); (10405 11854847 G A); (10406 11855059 A G); (10407 11855232 G A); (10408 11855245
T C); (10409 11855272 G A); (10410 11856254 A G); (10411 11856378 C T); (10412 11856450 G
T); (10413 11856467 C T); (10414 11857258 A T); (10415 11857571 T C); (10416 11857956 A G); (10417
11858333 G A); (10418 11858552 A G); (10419 11859130 A G); (10420 11859402 T G); (10421 11859534
G A); (10422 11860010 G A); (10423 11860089 G A); (10424 11860138 C T); (10425 11860164 T
C); (10426 11860174 G A); (10427 11860349 TGG T); (10428 11860651 T C); (10429 11860705 A
C); (10430 11860736 C G); (10431 11860769 T C); (10432 11860789 C
CGCGCTGATCGCGCCACTCTG); (10433 11860812 T G); (10434 11861070 A G); (10435 11861351 T
C); (10436 11861460 G A); (10437 11861751 G A); (10438 11861845 T C); (10439 11862031 C T); (10440
11862107 G A); (10441 11862128 C A); (10442 11862188 T TA); (10443 11862262 A G); (10444
11862362 C A); (10445 11862407 T C); (10446 11862572 T G); (10447 11863216 G A); (10448 11863404
T C); (10449 11863499 T A); (10450 11863763 T G); (10451 11864307 C T); (10452 11864403 G A); (10453
11864572 C T); (10454 11864609 G T); (10455 11864691 G A); (10456 11865574 C T); (10457 11865580
C A); (10458 11866012 C A); (10459 11866065 T G); (10460 11866135 T C); (10461 11866291 G
A); (10462 11866666 T C); (10463 11866816 G A); (10464 11866824 G A); (10465 11866852 G A); (10466
11867120 A C); (10467 11867186 T G); (10468 11867248 T C); (10469 11867412 G A); (10470 11867502
C A); (10471 11867603 C T); (10472 11867624 AC A); (10473 11867739 G A); (10474 11867805 A
G); (10475 11867827 C T); (10476 11867982 T A); (10477 11868197 C T); (10478 11868451 G T); (10479
11868474 C G); (10480 11868551 AATAT A); (10481 11868927 T C); (10482 11869136 C T); (10483
11870135 T G); (10484 11870201 C A); (10485 11870376 T C); (10486 11870422 G A); (10487 11870635
A G); (10488 11870748 G A); (10489 11870831 T TGGG); (10490 11870925 G A); (10491 11871035 T
C); (10492 11871072 G A); (10493 11871079 G A); (10494 11871126 T A); (10495 11871127 T G); (10496
11871212 C T); (10497 11871303 A G); (10498 11871331 T C); (10499 11871432 A G); (10500 11871441
T C); (10501 11871585 T A); (10502 11871616 A C); (10503 11871725 G A); (10504 11871742 G
GC); (10505 11871975 T G); (10506 11872179 C T); (10507 11872340 C T); (10508 11872382 C T); (10509
11872567 G A); (10510 11872903 G A); (10511 11872979 C T); (10512 11873003 C T); (10513 11873083
G A); (10514 11873156 C T); (10515 11873334 C T); (10516 11873452 C T); (10517 11873523 T C); (10518
11873568 A G); (10519 11873804 G A); (10520 11873965 C T); (10521 11874013 A G); (10522 11874116
C T); (10523 11874134 A G); (10524 11874211 C G); (10525 11874397 A G); (10526 11874583 T
C); (10527 11874690 T C); (10528 11874830 T C); (10529 11874854 G A); (10530 11875005 G A); (10531
11875051 T C); (10532 11875115 C T); (10533 11875127 G A); (10534 11875366 A G); (10535 11875367
G A); (10536 11875559 T C); (10537 11875696 A T); (10538 11875917 G T); (10539 11875945 T C); (10540
11876173 AT A); (10541 11876256 TG T); (10542 11876391 C T); (10543 11876531 G A); (10544
11876596 G GAACAAGACTCTTTTA); (10545 11876797 G A); (10546 11877172 G A); (10547 11877260 C
T); (10548 11877261 T C); (10549 11877521 A G); (10550 11877694 A G); (10551 11877744 G A); (10552
11877811 G T); (10553 11877834 G C); (10554 11877866 A AC); (10555 11877896 T G); (10556
11878064 G GT); (10557 11878751 T C); (10558 11878753 G A); (10559 11878764 T C); (10560 11878886
G A); (10561 11878925 A G); (10562 11878969 C T); (10563 11879247 G A); (10564 11879359 C
T); (10565 11879904 T G); (10566 11880142 A G); (10567 11880219 T C); (10568 11880466 T C); (10569
11880483 A G); (10570 11880508 C T); (10571 11880931 A C); (10572 11880939 A G); (10573 11880996
A G); (10574 11881320 A G); (10575 11881327 C T); (10576 11881381 A G); (10577 11882013 C
T); (10578 11882068 GCACA G); (10579 11882076 T A); (10580 11882280 T C); (10581 11882293 G
A); (10582 11882296 C T); (10583 11882297 T C); (10584 11882298 T C); (10585 11882374 G A); (10586
11882419 C T); (10587 11882483 C T); (10588 11882551 T C); (10589 11882560 C T); (10590 11882796 G
A); (10591 11882826 G A); (10592 11882858 G T); (10593 11883023 C G); (10594 11883169 C T); (10595
11883255 G A); (10596 11883302 G A); (10597 11883363 T C); (10598 11883424 A G); (10599 11883463
C A); (10600 11883608 T C); (10601 11883815 T A); (10602 11884061 T C); (10603 11884212 A T); (10604
11886054 A G); (10605 11886912 G A); (10606 11886917 A G); (10607 11887038 T C); (10608 11887481
G A); (10609 11888206 A G); (10610 11888302 G A); (10611 11888847 C T); (10612 11893700 G
A); (10613 11894749 C T); (10614 11894801 T G); (10615 11896325 C G); (10616 11896326 C T); (10617
11896362 TA T); (10618 11896471 C T); (10619 11896557 G A); (10620 11896702 C T); (10621 11897485
G A); (10622 11897516 A T); (10623 11897711 A G); (10624 11897762 A G); (10625 11897855 T
C); (10626 11898042 G A); (10627 11898350 C T); (10628 11898383 A G); (10629 11898401 G A); (10630
11898438 A T); (10631 11898953 T C); (10632 11899001 A G); (10633 11899655 G T); (10634 11899667
T C); (10635 11899785 A G); (10636 11899839 G A); (10637 11900487 A T); (10638 11901240 G
A); (10639 11901260 T C); (10640 11901287 C T); (10641 11901310 G A); (10642 11901350 T C); (10643
11901385 T C); (10644 11901747 C T); (10645 11902230 G A); (10646 11907493 A T); (10647 11907499 T
C); (10648 11907722 C A); (10649 11909556 A T); (10650 11909736 T C); (10651 11909773 G T); (10652
11909824 A G); (10653 11909946 A G); (10654 11910571 G A); (10655 11911778 A G); (10656 11911830
G A); (10657 11912135 A C); (10658 11912165 G A); (10659 11912197 C T); (10660 11912222 G
T); (10661 11912253 A G); (10662 11912281 A G); (10663 11912297 C T); (10664 11912346 C T); (10665
11912380 T C); (10666 11912471 C T); (10667 11912486 G A); (10668 11912634 A G); (10669 11912648
T C); (10670 11912950 T C); (10671 11913133 A G); (10672 11913244 A G); (10673 11913262 C
G); (10674 11913309 A G); (10675 11913696 T C); (10676 11914556 T C); (10677 11914565 C T); (10678
11914633 A G); (10679 11914760 T C); (10680 11914959 A G); (10681 11914995 T A); (10682 11915250
G T); (10683 11915411 G A); (10684 11915468 G A); (10685 11915533 T TACCTAAC); (10686 11915886 G
A); (10687 11916055 G A); (10688 11916175 C T); (10689 11916271 T C); (10690 11916361 C A); (10691
11916376 C T); (10692 11916987 A C); (10693 11917060 G T); (10694 11917101 T C); (10695 11917204
G A); (10696 11917376 G A); (10697 11917477 G A); (10698 11917538 G A); (10699 11917805 C
A); (10700 11917897 A AT); (10701 11918181 TAATG T); (10702 11918485 CT C); (10703 11918572 A
G); (10704 11918576 G A); (10705 11919438 T C); (10706 11919466 T C); (10707 11919798 G A); (10708
11919940 T C); (10709 11920547 T C); (10710 11920960 T A); (10711 11921511 T C); (10712 11921907 A
T); (10713 11922304 G A); (10714 11922414 C T); (10715 11922780 A T); (10716 11923009 A G); (10717
11923099 T G); (10718 11923113 T C); (10719 11923398 G A); (10720 11923538 G T); (10721 11923678
T A); (10722 11923705 T TA); (10723 11923717 G T); (10724 11923857 T TCATGAA); (10725 11923860 A
G); (10726 11924040 T C); (10727 11924052 G T); (10728 11924126 A G); (10729 11924250 C A); (10730
11924305 G GA); (10731 11924643 A T); (10732 11924723 G A); (10733 11924754 G T); (10734
11925251 G A); (10735 11925285 C T); (10736 11925747 C T); (10737 11925804 A G); (10738 11926028
T C); (10739 11926396 C T); (10740 11926493 A C); (10741 11926675 G A); (10742 11927399 A
G); (10743 11927432 C T); (10744 11927433 G A); (10745 11927509 A G); (10746 11927625 A G); (10747
11927870 C T); (10748 11928262 G A); (10749 11928303 G A); (10750 11928479 T C); (10751 11928700
T C); (10752 11929664 C T); (10753 11929962 G A); (10754 11930078 G A); (10755 11930126 G
A); (10756 11930346 C T); (10757 11930360 C T); (10758 11930623 C T); (10759 11930739 G A); (10760
11930759 C T); (10761 11930931 C T); (10762 11931014 A G); (10763 11931268 A C); (10764 11931493
G A); (10765 11931582 C CA); (10766 11931701 C T); (10767 11931834 T C); (10768 11931899 G
A); (10769 11932509 A C); (10770 11932625 T G); (10771 11932797 G T); (10772 11932924 A G); (10773
11932925 A T); (10774 11933011 G T); (10775 11933258 T A); (10776 11933386 T G); (10777 11933424
C T); (10778 11933607 G A); (10779 11933670 T TAGATCA); (10780 11934476 G A); (10781 11934658 A
T); (10782 11934660 A AAGTT); (10783 11935231 G A); (10784 11935492 C A); (10785 11935979 T
C); (10786 11936170 T C); (10787 11936187 C G); (10788 11936651 G A); (10789 11936706 T G); (10790
11936804 C CAT); (10791 11937439 C T); (10792 11937945 A G); (10793 11937956 T C); (10794
11938107 A G); (10795 11938142 A C); (10796 11939964 T C); (10797 11940210 A G); (10798 11940277
A G); (10799 11940296 G A); (10800 11940399 C T); (10801 11940458 T C); (10802 11940570 A
G); (10803 11940974 A G); (10804 11941183 C A); (10805 11941356 C T); (10806 11941434 C T); (10807
11941534 T C); (10808 11941642 T C); (10809 11941668 A G); (10810 11941732 A G); (10811 11941735
G A); (10812 11941829 G A); (10813 11941877 A G); (10814 11942059 T C); (10815 11942287 G
A); (10816 11942543 G A); (10817 11943230 A G); (10818 11944074 G A); (10819 11944515 C T); (10820
11944651 A C); (10821 11945426 C A); (10822 11945631 G A); (10823 11945792 C CAAA); (10824
11946303 G A); (10825 11946367 A G); (10826 11946495 T C); (10827 11946503 G A); (10828 11946642
C T); (10829 11946806 T C); (10830 11947176 A G); (10831 11947438 T TAGACGAGAATGGGTC); (10832
11948074 T C); (10833 11948363 T A); (10834 11948381 C T); (10835 11948505 A T); (10836 11948789 T
C); (10837 11948874 T A); (10838 11949204 T C); (10839 11949244 G A); (10840 11950154 C T); (10841
11950251 A C); (10842 11952257 G A); (10843 11953463 G C); (10844 11953587 G A); (10845 11955993
T C); (10846 11957739 A T); (10847 11958666 C T); (10848 11959037 G T); (10849 11961520 T C); (10850
11961555 ATGGTGAATG A); (10851 11961568 C T); (10852 11961622 A G); (10853 11962179 G
A); (10854 11962377 C T); (10855 11963381 T C); (10856 11964130 G T); (10857 11964407 C T); (10858
11964599 A G); (10859 11965148 A T); (10860 11965946 T TA); (10861 11965988 A G); (10862
11966024 G A); (10863 11966132 G A); (10864 11966322 C T); (10865 11966364 A G); (10866 11966672
T C); (10867 11967230 A G); (10868 11967422 A G); (10869 11967488 G A); (10870 11968602 C
A); (10871 11970389 G A); (10872 11970414 T C); (10873 11970752 G A); (10874 11971063 G A); (10875
11971317 A C); (10876 11971669 G A); (10877 11972101 A T); (10878 11972706 G A); (10879 11973073
A G); (10880 11973167 A G); (10881 11973241 A G); (10882 11973279 A G); (10883 11973302 C
T); (10884 11973583 G A); (10885 11973658 T C); (10886 11973687 T C); (10887 11973817 G T); (10888
11973887 T C); (10889 11973896 G A); (10890 11973971 T G); (10891 11974000 A G); (10892 11974092
T C); (10893 11974115 G A); (10894 11974145 G T); (10895 11974587 G A); (10896 11974761 C
T); (10897 11974889 C T); (10898 11975208 T TAAGAGCTAAAAG); (10899 11975284 G A); (10900
11975310 C A); (10901 11975363 A G); (10902 11975394 C T); (10903 11975550 A G); (10904 11975582
C T); (10905 11976098 T G); (10906 11976186 C T); (10907 11976247 G A); (10908 11976301 T C); (10909
11976303 A G); (10910 11976449 T C); (10911 11976512 C T); (10912 11976952 A G); (10913 11977168
C T); (10914 11977174 G T); (10915 11977177 A G); (10916 11977268 T G); (10917 11977376 T
C); (10918 11977386 T C); (10919 11977614 A T); (10920 11977680 C T); (10921 11978011 T C); (10922
11978266 C T); (10923 11978383 C T); (10924 11978663 G A); (10925 11978804 C T); (10926 11979395 C
T); (10927 11980153 G A); (10928 11980294 T C); (10929 11980543 G A); (10930 11980882 T C); (10931
11981060 T G); (10932 11981133 A G); (10933 11981410 C T); (10934 11981517 G A); (10935 11981546
T G); (10936 11982167 G A); (10937 11982200 G A); (10938 11982243 G A); (10939 11982285 A
G); (10940 11982554 C T); (10941 11982602 T C); (10942 11984036 T C); (10943 11985836 T C); (10944
11985892 T C); (10945 11985945 A G); (10946 11986297 C T); (10947 11986524 C T); (10948 11986915 T
G); (10949 11987040 A G); (10950 11987215 T C); (10951 11988223 A G); (10952 11988570 C T); (10953
11989064 T C); (10954 11989082 C T); (10955 11989270 CAT C); (10956 11989566 C G); (10957
11989742 A G); (10958 11989813 G A); (10959 11990036 C T); (10960 11990332 A G); (10961 11990357
G A); (10962 11991528 A G); (10963 11991783 A C); (10964 11991819 C T); (10965 11992059 T
C); (10966 11992076 G A); (10967 11992498 T A); (10968 11992795 A G); (10969 11992995 C T); (10970
11993157 G C); (10971 11993346 T C); (10972 11993478 T C); (10973 11993713 C T); (10974 11993750 T
TA); (10975 11994091 G C); (10976 11994516 C G); (10977 11994551 A T); (10978 11994733 A
AC); (10979 11994912 C T); (10980 11994939 A C); (10981 11995213 C A); (10982 11995624 G C); (10983
11995808 A T); (10984 11995935 A C); (10985 11996417 A C); (10986 11997166 A G); (10987 11997242
A G); (10988 11997400 A G); (10989 11997472 G A); (10990 11997883 C T); (10991 11998069 C
T); (10992 11998919 G T); (10993 11999128 G A); (10994 11999130 T C); (10995 12001407 C T); (10996
12001474 G A); (10997 12001843 G A); (10998 12002001 G A); (10999 12002849 T C); (11000 12003154
C T); (11001 12003406 T C); (11002 12003673 T C); (11003 12003694 T C); (11004 12003731 C T); (11005
12003771 C T); (11006 12003912 C T); (11007 12003916 C T); (11008 12004815 C T); (11009 12004943 C
T); (11010 12005748 T C); (11011 12006245 T G); (11012 12006246 T G); (11013 12006266 A T); (11014
12006267 T A); (11015 12006346 T C); (11016 12006664 T G); (11017 12007044 T A); (11018 12007058
A AC); (11019 12007132 C A); (11020 12007181 G A); (11021 12007292 A G); (11022 12007299 T
C); (11023 12007396 G A); (11024 12007509 C T); (11025 12007760 TG T); (11026 12007838 A G); (11027
12007901 A G); (11028 12008076 C T); (11029 12011032 G A); (11030 12011445 G A); (11031 12011461
G T); (11032 12011579 C T); (11033 12011797 A G); (11034 12012015 C T); (11035 12012163 C
G); (11036 12012305 C A); (11037 12012360 CATTT C); (11038 12012483 T C); (11039 12012800 A
T); (11040 12012999 T C); (11041 12013016 A G); (11042 12013348 C A); (11043 12013372 C T); (11044
12016778 T C); (11045 12017339 C T); (11046 12017559 G A); (11047 12017766 C T); (11048 12018156
A C); (11049 12018685 G T); (11050 12018779 G A); (11051 12018853 A T); (11052 12018988 C
T); (11053 12019008 A G); (11054 12019796 A G); (11055 12020084 T C); (11056 12020456 C T); (11057
12021053 A G); (11058 12021496 T C); (11059 12022866 C T); (11060 12022955 CT C); (11061 12023081
C T); (11062 12023607 A G); (11063 12024211 T G); (11064 12025399 T C); (11065 12025433 TCTAG
T); (11066 12026608 T C); (11067 12026804 C A); (11068 12027004 C T); (11069 12027194 A C); (11070
12027468 G A); (11071 12027498 C T); (11072 12027601 G A); (11073 12027634 T C); (11074 12027688
C T); (11075 12027733 A G); (11076 12028252 G A); (11077 12028540 G A); (11078 12028623 T
C); (11079 12028678 G C); (11080 12028850 T C); (11081 12028959 G A); (11082 12028985 G A); (11083
12029056 C T); (11084 12029164 C CAACG); (11085 12029711 A G); (11086 12029801 G A); (11087
12030440 AC A); (11088 12030804 TA T); (11089 12031388 C A); (11090 12031407
ACGTTTATATTTAAGCAAT A); (11091 12032214 T C); (11092 12032929 C T); (11093 12033379
GTATTTAAACCTA G); (11094 12033606 T A); (11095 12033610 TTA T); (11096 12035267 A G); (11097
12035312 G A); (11098 12035339 C T); (11099 12035648 A T); (11100 12035688 G A); (11101 12035730
A G); (11102 12036234 A G); (11103 12036373 C T); (11104 12036419 T C); (11105 12036471 A
G); (11106 12036656 G A); (11107 12036688 G A); (11108 12036765 G A); (11109 12036783 C T); (11110
12036877 A G); (11111 12037046 A C); (11112 12037124 T A); (11113 12037137 C T); (11114 12037191
T C); (11115 12037320 C T); (11116 12037434 G T); (11117 12037486 T C); (11118 12037491 C T); (11119
12037627 A G); (11120 12037682 A G); (11121 12038107 T C); (11122 12038150 A G); (11123 12038181
A G); (11124 12038354 T C); (11125 12038362 C T); (11126 12038436 T C); (11127 12038451 G
A); (11128 12038602 C A); (11129 12038783 T C); (11130 12039048 C T); (11131 12039174 C T); (11132
12039384 C G); (11133 12039393 C T); (11134 12041953 A G); (11135 12042040 G A); (11136 12042100
T G); (11137 12042144 G A); (11138 12042241 A G); (11139 12042331 G C); (11140 12042598 G
A); (11141 12042601 AT A); (11142 12042933 C T); (11143 12042960 G A); (11144 12043207 A C); (11145
12043226 T A); (11146 12043318 C T); (11147 12043350 G T); (11148 12043966 A G); (11149 12044065
A G); (11150 12044081 T C); (11151 12044166 A G); (11152 12044172 T G); (11153 12044284 A
G); (11154 12044342 T C); (11155 12044407 G A); (11156 12044414 G A); (11157 12044420 G A); (11158
12044426 G T); (11159 12044459 G A); (11160 12044570 T A); (11161 12044652 C T); (11162 12044738
T C); (11163 12044739 A C); (11164 12044750 C T); (11165 12044843 T C); (11166 12044879 G A); (11167
12044890 T C); (11168 12044953 T C); (11169 12045072 T C); (11170 12045119 G A); (11171 12045121 T
C); (11172 12045144 A C); (11173 12045352 G A); (11174 12045371 G A); (11175 12045396 G A); (11176
12045417 T C); (11177 12045422 C T); (11178 12045547 C T); (11179 12045579 T C); (11180 12045662 G
A); (11181 12045837 C T); (11182 12045977 T C); (11183 12046258 A G); (11184 12046277 C T); (11185
12046576 G C); (11186 12046607 G A); (11187 12046631 T G); (11188 12046785 G A); (11189 12046859
G C); (11190 12046916 T C); (11191 12047097 G A); (11192 12047483 C T); (11193 12047585 A
G); (11194 12047604 T C); (11195 12047746 G A); (11196 12047859 G A); (11197 12048742 T TA); (11198
12048824 A G); (11199 12049419 TTA T); (11200 12049521 G T); (11201 12049888 T A); (11202
12050137 CA C); (11203 12050196 G GA); (11204 12050679 A AT); (11205 12050799 T C); (11206
12050893 G GGTTCAGCTGGTACTGATCGAATTC); (11207 12051195 TTA T); (11208 12051318 A
G); (11209 12051559 C T); (11210 12053450 G T); (11211 12053789 G T); (11212 12054868 AT A); (11213
12054928 T TA); (11214 12055225 A T); (11215 12055343 C G); (11216 12055891 C T); (11217 12056009
C T); (11218 12056036 G A); (11219 12056079 A G); (11220 12056269 A T); (11221 12056366 G
T); (11222 12056925 G T); (11223 12056993 G GATA); (11224 12057035 G A); (11225 12057208 G
A); (11226 12057294 C T); (11227 12057776 G A); (11228 12057794 T A); (11229 12057797 C T); (11230
12057859 C T); (11231 12057892 C T); (11232 12058032 TA T); (11233 12058417 A G); (11234 12058444
T C); (11235 12058982 C T); (11236 12059127 G A); (11237 12059158 A G); (11238 12059300 A
G); (11239 12059455 G T); (11240 12059533 C T); (11241 12059570 A G); (11242 12059601 C A); (11243
12059867 G A); (11244 12059875 G GC); (11245 12059936 G A); (11246 12060069 A G); (11247
12060110 T C); (11248 12060112 G C); (11249 12060140 C G); (11250 12060196
GGCAAAAAGAGGAGAATTAA G); (11251 12060357 T C); (11252 12060364 A G); (11253 12060607 AC
A); (11254 12060950 C A); (11255 12061153 A C); (11256 12061489 T C); (11257 12061586 G A); (11258
12061888 A G); (11259 12063597 A G); (11260 12063634 C T); (11261 12063682 C A); (11262 12063906
C T); (11263 12064079 T C); (11264 12064086 A G); (11265 12064613 G A); (11266 12065132 TA
T); (11267 12065338 A G); (11268 12065694 G A); (11269 12065773 T C); (11270 12066781 C T); (11271
12066796 C T); (11272 12066863 C T); (11273 12066976 T A); (11274 12067038 C T); (11275 12067173 T
G); (11276 12067301 T C); (11277 12067303 C T); (11278 12067401 G C); (11279 12067615 T C); (11280
12067871 C G); (11281 12067953 G A); (11282 12068040 T G); (11283 12068231 C T); (11284 12069340
A G); (11285 12069464 G T); (11286 12069496 C T); (11287 12070525 T C); (11288 12070967 C
G); (11289 12071263 T C); (11290 12071521 C T); (11291 12071552 A G); (11292 12071589 G A); (11293
12071620 C T); (11294 12071632 A G); (11295 12071798 T C); (11296 12071962 C T); (11297 12075985 C
G); (11298 12076673 T A); (11299 12078859 GGTATACGTATTTGT G); (11300 12079466 A T); (11301
12079469 C A); (11302 12079486 C G); (11303 12080023 A G); (11304 12080038 A G); (11305 12080044
G A); (11306 12080278 T A); (11307 12080325 G A); (11308 12080995 C A); (11309 12081097 T
C); (11310 12081357 T A); (11311 12081581 C T); (11312 12081615 C T); (11313 12081657 A C); (11314
12081766 C T); (11315 12081911 A G); (11316 12082099 CAAT C); (11317 12082292 G C); (11318
12082311 A G); (11319 12082902 C T); (11320 12083078 A G); (11321 12083668 G A); (11322 12083789
A G); (11323 12084283 G A); (11324 12084420 G C); (11325 12085135 C T); (11326 12085321 A
G); (11327 12086107 G A); (11328 12087132 A G); (11329 12087217 G A); (11330 12087534 G A); (11331
12087565 T C); (11332 12088522 G A); (11333 12088770 ACTAGAGGTAGTG A); (11334 12089202 T
C); (11335 12089418 A T); (11336 12090443 G A); (11337 12090528 C T); (11338 12090823 G A); (11339
12090875 G A); (11340 12091343 A G); (11341 12091361 A G); (11342 12091438 C A); (11343 12091651
A G); (11344 12091713 T C); (11345 12092116 A G); (11346 12092118 C T); (11347 12092787 C
A); (11348 12092997 T C); (11349 12093153 A G); (11350 12093216 A C); (11351 12094036 G A); (11352
12094064 T C); (11353 12094572 G A); (11354 12094715 C T); (11355 12094784 G A); (11356 12095232
G A); (11357 12095541 G T); (11358 12095672 G C); (11359 12096397 G A); (11360 12096626 C
T); (11361 12096645 T C); (11362 12096680 G A); (11363 12097015 T G); (11364 12097444 C T); (11365
12097570 A C); (11366 12097584 A C); (11367 12097672 G A); (11368 12097711 A G); (11369 12097806
C T); (11370 12097928 C A); (11371 12098114 T C); (11372 12098222 G C); (11373 12098411 T C); (11374
12100044 G A); (11375 12100114 C A); (11376 12100286 C T); (11377 12100392 A C); (11378 12100400
A G); (11379 12101080 T C); (11380 12101370 C T); (11381 12101679 G A); (11382 12101811 C
T); (11383 12101901 C T); (11384 12102074 C A); (11385 12102371 G A); (11386 12102374 T C); (11387
12102869 T C); (11388 12102940 T C); (11389 12103539 C T); (11390 12103587 T C); (11391 12103643 C
T); (11392 12103704 T C); (11393 12103807 A G); (11394 12103865 G A); (11395 12103880 G T); (11396
12104354 C T); (11397 12104677 A G); (11398 12104679 C T); (11399 12104681 G A); (11400 12105017
A T); (11401 12105733 A T); (11402 12105954 A G); (11403 12106088 A G); (11404 12106178 C
A); (11405 12106397 A G); (11406 12106780 C T); (11407 12106983 T A); (11408 12107175 A G); (11409
12107565 A C); (11410 12108360 G A); (11411 12108883 TTT AAA); (11412 12109062 T C); (11413
12109067 T C); (11414 12109465 C T); (11415 12109537 T A); (11416 12109929 G A); (11417 12110392
C A); (11418 12110439 A G); (11419 12110719 A C); (11420 12111970 T C); (11421 12112191 G
A); (11422 12112289 A G); (11423 12113179 A G); (11424 12113206 A G); (11425 12113393 A T); (11426
12113588 C CT); (11427 12114282 A T); (11428 12115492 T C); (11429 12116743 G A); (11430 12118526
T C); (11431 12118531 A G); (11432 12118733 C T); (11433 12119161 G C); (11434 12119499 A
G); (11435 12119800 G A); (11436 12119868 C T); (11437 12119967 T G); (11438 12119987 T C); (11439
12120012 A G); (11440 12120409 A G); (11441 12120785 A G); (11442 12120878 G C); (11443 12120906
A G); (11444 12120923 G A); (11445 12121158 A G); (11446 12121218 G A); (11447 12121399 A
T); (11448 12121735 A G); (11449 12121741 T C); (11450 12122016 T C); (11451 12122410 T C); (11452
12123030 T C); (11453 12123071 A G); (11454 12123102 C T); (11455 12125919 A G); (11456 12126291
C T); (11457 12126306 A G); (11458 12126346 A G); (11459 12126416 G C); (11460 12126422 A
G); (11461 12126758 G A); (11462 12126894 A C); (11463 12126917 C T); (11464 12126920 A G); (11465
12126933 G T); (11466 12127096 G A); (11467 12127190 A C); (11468 12127234 A C); (11469 12127284
C T); (11470 12127979 A T); (11471 12128027 G A); (11472 12128341 G A); (11473 12128353 G
C); (11474 12128387 C A); (11475 12128474 C T); (11476 12128666 A G); (11477 12128761 T C); (11478
12129090 T A); (11479 12129175 A G); (11480 12129425 A G); (11481 12129495 A AT); (11482
12129556 T A); (11483 12129934 CCG C); (11484 12129988 A C); (11485 12130496 A G); (11486
12130687 CT C); (11487 12132321 T C); (11488 12132535 C CTTTAA); (11489 12133447 A G); (11490
12133550 A T); (11491 12133875 A G); (11492 12134158 C A); (11493 12134247 A AAT); (11494
12134307 A AC); (11495 12134846 C CT); (11496 12135505 A T); (11497 12135744 G T); (11498
12135948 G A); (11499 12136109 G A); (11500 12136168 C G); (11501 12136859 A G); (11502 12137143
C T); (11503 12137251 C T); (11504 12137300 G A); (11505 12137506 T C); (11506 12137517 G C); (11507
12137531 C T); (11508 12137611 G A); (11509 12137756 A G); (11510 12137878 T C); (11511 12138518
T C); (11512 12138671 C A); (11513 12139456 C T); (11514 12139599 C T); (11515 12139735 G A); (11516
12139850 A G); (11517 12141075 CTTA C); (11518 12141234 G C); (11519 12141777 C T); (11520
12141805 C T); (11521 12141876 G A); (11522 12142159 A G); (11523 12142570 T C); (11524 12142585
A T); (11525 12142618 C A); (11526 12142632 T C); (11527 12143169 C T); (11528 12143600 A G); (11529
12143616 T A); (11530 12144009 G A); (11531 12144042 T C); (11532 12144669 C A); (11533 12144882
GA G); (11534 12145362 G A); (11535 12145794 G A); (11536 12146383 AAG A); (11537 12146601 T
G); (11538 12146893 A G); (11539 12146916 T A); (11540 12146959 G GA); (11541 12147059 CT
C); (11542 12147082 T A); (11543 12147221 A G); (11544 12147352 A T); (11545 12147443 T C); (11546
12147621 A G); (11547 12147894 A G); (11548 12148458 C T); (11549 12148887 C T); (11550 12148919
CA C); (11551 12149121 T C); (11552 12149189 G A); (11553 12149254 T G); (11554 12149330 C
T); (11555 12149468 T C); (11556 12150443 G C); (11557 12150544 T G); (11558 12150590 C T); (11559
12150744 A G); (11560 12150807 T G); (11561 12150846 A G); (11562 12150959 G A); (11563 12151528
G A); (11564 12151599 G A); (11565 12151612 G T); (11566 12152147 G T); (11567 12152341 A
G); (11568 12152440 G A); (11569 12152494 G T); (11570 12152530 T C); (11571 12152992 C T); (11572
12153655 T C); (11573 12153678 G A); (11574 12153732 A G); (11575 12153862 ATACT A); (11576
12154163 A C); (11577 12154273 T C); (11578 12154292 T C); (11579 12154318 G C); (11580 12154512
G A); (11581 12154537 G A); (11582 12155104 A G); (11583 12155130 C T); (11584 12155597 A
G); (11585 12156114 G T); (11586 12156223 T G); (11587 12156861 T C); (11588 12156912 G A); (11589
12157433 T C); (11590 12157640 AT A); (11591 12157835 G A); (11592 12158060 T C); (11593 12158082
T C); (11594 12158093 C T); (11595 12158140 A G); (11596 12158154 T G); (11597 12158192 A
G); (11598 12158198 C T); (11599 12158202 A G); (11600 12158215 C T); (11601 12158595 T C); (11602
12159731 A T); (11603 12159742 GGGT G); (11604 12162913 GA G); (11605 12162988 A T); (11606
12163589 A G); (11607 12163887 T C); (11608 12163943 G A); (11609 12163966 C T); (11610 12163970
C T); (11611 12164355 A AATATAT); (11612 12164458 T A); (11613 12164461 A G); (11614 12164462 G
T); (11615 12164463 T A); (11616 12164574 TA T); (11617 12164894 C A); (11618 12165321 A G); (11619
12165570 A AAATTATCTACAATCT); (11620 12165774 T C); (11621 12166007 T C); (11622 12166114 G
A); (11623 12166119 G A); (11624 12166133 AC A); (11625 12166379 A G); (11626 12166728 A
T); (11627 12166915 A G); (11628 12167229 G A); (11629 12167294 T C); (11630 12167383 G A); (11631
12167528 G A); (11632 12167762 C A); (11633 12167953 G A); (11634 12168327 G A); (11635 12168698
G A); (11636 12168774 A G); (11637 12169191 T A); (11638 12169490 A G); (11639 12169681 A
G); (11640 12169724 C A); (11641 12169733 T G); (11642 12169754 T C); (11643 12170309 T C); (11644
12170435 T C); (11645 12170744 C T); (11646 12170999 C T); (11647 12171347 C T); (11648 12171351 T
G); (11649 12171601 A T); (11650 12171728 G A); (11651 12171819 C T); (11652 12172872 G A); (11653
12172957 C T); (11654 12172995 A C); (11655 12173055 T C); (11656 12173233 T C); (11657 12173560 A
T); (11658 12173660 G A); (11659 12174319 C T); (11660 12174391 A T); (11661 12175361 C T); (11662
12175805 C T); (11663 12176237 C G); (11664 12176499 G A); (11665 12176674 A G); (11666 12177037
T C); (11667 12177187 A G); (11668 12177230 T C); (11669 12177293 T C); (11670 12178862 G
A); (11671 12180387 C T); (11672 12181778 T C); (11673 12186783 A G); (11674 12186938 C T); (11675
12186953 C T); (11676 12187024 G A); (11677 12187177 C T); (11678 12187265 T C); (11679 12187381 T
C); (11680 12187394 T C); (11681 12187709 A G); (11682 12189015 T A); (11683 12189099 G T); (11684
12189124 T C); (11685 12189286 C A); (11686 12189315 A G); (11687 12189454 C T); (11688 12190042
A G); (11689 12190157 C T); (11690 12190509 C T); (11691 12190599 G A); (11692 12190690 A
G); (11693 12190735 G A); (11694 12190910 C T); (11695 12191261 ATTT A); (11696 12191333 A
T); (11697 12191521 C T); (11698 12191638 T C); (11699 12191987 T G); (11700 12192366 C A); (11701
12192599 A C); (11702 12192833 C G); (11703 12193271 A C); (11704 12193407 A G); (11705 12193605
G A); (11706 12193649 C T); (11707 12194362 A G); (11708 12194405 C T); (11709 12195445 G
A); (11710 12195611 T C); (11711 12195708 C T); (11712 12195889 T C); (11713 12196016 C T); (11714
12196415 A G); (11715 12196751 G A); (11716 12196950 C T); (11717 12197619 T C); (11718 12197776
A G); (11719 12197989 C T); (11720 12198240 GA G); (11721 12198574 T C); (11722 12198800 T
C); (11723 12199084 T C); (11724 12199236 G A); (11725 12199240 T C); (11726 12199316 T G); (11727
12199372 G A); (11728 12200008 G C); (11729 12200146 A G); (11730 12200307 CTTT C); (11731
12200355 C T); (11732 12200566 A G); (11733 12200808 C T); (11734 12201512 G A); (11735 12201636
C T); (11736 12201703 C T); (11737 12201734 A G); (11738 12201767 T C); (11739 12202183 G
A); (11740 12202517 G A); (11741 12202797 G A); (11742 12203541 G A); (11743 12203672 T C); (11744
12203752 G T); (11745 12203849 C T); (11746 12204057 T G); (11747 12204127 C T); (11748 12204135 T
C); (11749 12205022 T C); (11750 12205031 G A); (11751 12205495 A G); (11752 12205894 AT A); (11753
12206063 C T); (11754 12206276 A T); (11755 12207053 T TA); (11756 12207801 C G); (11757 12208241
T A); (11758 12209001 C A); (11759 12209309 A G); (11760 12209669 G GT); (11761 12211274 A
G); (11762 12211303 T C); (11763 12211591 G A); (11764 12212190 C T); (11765 12213070 C T); (11766
12213557 G A); (11767 12213927 A C); (11768 12214296 G A); (11769 12216228 A G); (11770 12216377
A T); (11771 12217076 A G); (11772 12217220 T A); (11773 12217259 T A); (11774 12218138 G
A); (11775 12218167 C T); (11776 12218181 G A); (11777 12218195 A G); (11778 12218205 C G); (11779
12218363 G A); (11780 12218462 G A); (11781 12218678 T C); (11782 12219170 C T); (11783 12219195
C T); (11784 12219262 A ATC); (11785 12219291 T G); (11786 12219712 T C); (11787 12220074 A
G); (11788 12220306 G A); (11789 12220511 C T); (11790 12220921 G A); (11791 12221143 T G); (11792
12221171 G A); (11793 12221923 T C); (11794 12221952 G T); (11795 12222602 G T); (11796 12223749
G A); (11797 12224695 G T); (11798 12224995 G T); (11799 12225658 TA T); (11800 12226743 C
T); (11801 12226848 C T); (11802 12226905 A G); (11803 12226996 C T); (11804 12227145 A G); (11805
12227254 C T); (11806 12227504 T G); (11807 12227808 T TA); (11808 12227827 C T); (11809 12227831
A C); (11810 12227863 A G); (11811 12227912 T C); (11812 12227915 A G); (11813 12227989 A
G); (11814 12228629 C G); (11815 12229870 G A); (11816 12230194 C A); (11817 12230694 G A); (11818
12230934 C T); (11819 12231145 G A); (11820 12232709 G A); (11821 12232737 T C); (11822 12232832
C T); (11823 12233077 C T); (11824 12233402 C T); (11825 12233789 G A); (11826 12233961 C T); (11827
12234081 T C); (11828 12234109 A C); (11829 12234199 C A); (11830 12234269 G A); (11831 12235239
T G); (11832 12235892 C G); (11833 12236218 T G); (11834 12236239 A G); (11835 12236428 G
A); (11836 12236470 C T); (11837 12237086 A C); (11838 12237764 C A); (11839 12238276 T A); (11840
12238606 G T); (11841 12238658 AT A); (11842 12239248 G A); (11843 12240197 A G); (11844
12241008 G A); (11845 12241282 C T); (11846 12241302 C T); (11847 12242086 C T); (11848 12242737
GA); (11849 12243007 C T); (11850 12243022 A G); (11851 12243326 G A); (11852 12243464 A
G); (11853 12243505 C T); (11854 12244524 T C); (11855 12244543 C T); (11856 12244753 T C); (11857
12245096 G A); (11858 12245987 ATT A); (11859 12246860 C T); (11860 12247203 T C); (11861
12247707 A T); (11862 12247732 GGTT G); (11863 12248688 G A); (11864 12249426 A G); (11865
12251399 C T); (11866 12251411 G T); (11867 12252112 A G); (11868 12252677 T G); (11869 12252818
A T); (11870 12253222 A AT); (11871 12253371 T C); (11872 12253579 C A); (11873 12253611 A
C); (11874 12253895 T C); (11875 12254071 T C); (11876 12254146 TG T); (11877 12254150 G T); (11878
12255015 A C); (11879 12255161 A T); (11880 12255661 T C); (11881 12255755 C CA); (11882 12255862
A G); (11883 12255901 G A); (11884 12255960 A T); (11885 12256277 T G); (11886 12256452 AT
A); (11887 12256567 T A); (11888 12256886 G A); (11889 12256948 A G); (11890 12257053 C A); (11891
12257289 G A); (11892 12257295 G A); (11893 12257370 G A); (11894 12257397 G A); (11895 12257415
G A); (11896 12257506 T C); (11897 12258121 G C); (11898 12258350 A G); (11899 12258351 T
C); (11900 12258432 G A); (11901 12258508 C T); (11902 12258582 C T); (11903 12258628 A G); (11904
12258743 A ATT); (11905 12258887 T TA); (11906 12259248 T C); (11907 12259304 A G); (11908
12259464 C T); (11909 12259476 C T); (11910 12259477 G A); (11911 12259520 A C); (11912 12261446
C T); (11913 12261717 C T); (11914 12261782 G T); (11915 12262232 T C); (11916 12262619 G A); (11917
12262744 A T); (11918 12262839 C T); (11919 12262991 A G); (11920 12263008 C T); (11921 12263078
A T); (11922 12263216 T G); (11923 12263424 G A); (11924 12263950 T C); (11925 12264121 C
A); (11926 12264179 C T); (11927 12264210 A G); (11928 12264216 T C); (11929 12264261 T A); (11930
12264279 T C); (11931 12264300 G A); (11932 12264858 T G); (11933 12264962 T C); (11934 12265048
C T); (11935 12265800 A G); (11936 12265905 C A); (11937 12266051 C A); (11938 12266270 C
T); (11939 12266287 TA T); (11940 12266828 C T); (11941 12267435 C T); (11942 12267694 T C); (11943
12267793 G A); (11944 12267833 T C); (11945 12267910 A G); (11946 12267961 C T); (11947 12267985
G C); (11948 12268121 G A); (11949 12269313 C T); (11950 12269445 C G); (11951 12269506 C
T); (11952 12269638 G A); (11953 12270045 C T); (11954 12270227 C T); (11955 12270714 C T); (11956
12273624 G A); (11957 12273648 T G); (11958 12273659 G A); (11959 12276687 A G); (11960 12276973
T C); (11961 12277137 T C); (11962 12277204 C T); (11963 12277377 G A); (11964 12277432 C A); (11965
12277507 C T); (11966 12277569 T C); (11967 12277593 G A); (11968 12277718 G A); (11969 12277862
T C); (11970 12278139 G T); (11971 12278185 T C); (11972 12278233 A G); (11973 12278254 T
G); (11974 12278290 A G); (11975 12278364 T C); (11976 12278407 C T); (11977 12278490 G A); (11978
12278497 A G); (11979 12278520 C A); (11980 12279163 C T); (11981 12279308 G A); (11982 12279343
C T); (11983 12279380 G T); (11984 12279471 G A); (11985 12279508 A G); (11986 12279744 C
T); (11987 12279806 T C); (11988 12279979 G C); (11989 12280061 G T); (11990 12280117 G A); (11991
12280356 G T); (11992 12280856 T C); (11993 12280912 C T); (11994 12280920 T G); (11995 12281615
A G); (11996 12281645 G A); (11997 12281735 T C); (11998 12281893 C T); (11999 12281972 T
C); (12000 12282008 T C); (12001 12282164 C T); (12002 12282343 A T); (12003 12283258 G A); (12004
12284592 A C); (12005 12284646 A G); (12006 12285021 A ATC); (12007 12285983 T C); (12008
12286102 A G); (12009 12286240 A G); (12010 12286466 T C); (12011 12286473 G A); (12012 12286486
G A); (12013 12286711 C G); (12014 12287506 G A); (12015 12288933 T A); (12016 12288945 C
T); (12017 12289381 T G); (12018 12290029 C G); (12019 12290432 C T); (12020 12290464 G T); (12021
12290494 A T); (12022 12290507 G A); (12023 12290531 G A); (12024 12290698 C T); (12025 12291063
C A); (12026 12291940 A T); (12027 12292010 C T); (12028 12292051 G A); (12029 12292177 T C); (12030
12292242 A C); (12031 12292255 T C); (12032 12292292 T C); (12033 12292349 T G); (12034 12292600 C
T); (12035 12292833 C T); (12036 12292877 G C); (12037 12293491 T A); (12038 12293537 T C); (12039
12293603 A G); (12040 12293868 T C); (12041 12293881 C T); (12042 12294080 T C); (12043 12294280
A G); (12044 12294344 C T); (12045 12294363 G A); (12046 12294830 C T); (12047 12294862 C
G); (12048 12295353 C T); (12049 12295462 C T); (12050 12295490 T A); (12051 12295680 A G); (12052
12295827 C A); (12053 12296211 T C); (12054 12296333 A G); (12055 12296376 A G); (12056 12296708
A C); (12057 12297294 T A); (12058 12297597 A C); (12059 12297642 T C); (12060 12297729 T G); (12061
12297816 C T); (12062 12298177 G A); (12063 12298558 A G); (12064 12299011 A G); (12065 12299605
A T); (12066 12299677 G A); (12067 12300940 T C); (12068 12301211 G T); (12069 12301300 A
C); (12070 12301964 A AAT); (12071 12302055 A G); (12072 12302560 G A); (12073 12302874 T
A); (12074 12303142 C CA); (12075 12303194 G GA); (12076 12303465 G A); (12077 12303575 A
G); (12078 12303970 T G); (12079 12304436 G T); (12080 12304496 CA C); (12081 12304856 A
G); (12082 12305232 G A); (12083 12305265 G A); (12084 12305605 AT A); (12085 12305748 C
A); (12086 12306084 C T); (12087 12306641 T A); (12088 12307034 C T); (12089 12307649 G A); (12090
12307908 A G); (12091 12308139 G A); (12092 12308266 A G); (12093 12308435 C T); (12094 12308546
A G); (12095 12308571 T C); (12096 12308591 G A); (12097 12308678 G A); (12098 12308689 G
A); (12099 12309255 C T); (12100 12309369 A C); (12101 12309411 T C); (12102 12309453 T C); (12103
12309465 C T); (12104 12309536 G A); (12105 12309577 C T); (12106 12309870 G A); (12107 12309988
A G); (12108 12310809 G A); (12109 12310904 G T); (12110 12311012 T G); (12111 12311555 T
C); (12112 12311811 T TG); (12113 12311874 G T); (12114 12311894 C A); (12115 12312292 T C); (12116
12312472 G T); (12117 12313468 GA G); (12118 12314890 A G); (12119 12314922 C T); (12120
12315170 A G); (12121 12315372 T G); (12122 12315655 T C); (12123 12316127 C A); (12124 12316464
A G); (12125 12316654 A C); (12126 12316926 T A); (12127 12317046 A G); (12128 12317128 G
A); (12129 12317411 T G); (12130 12317580 A ATAAT); (12131 12317814 G A); (12132 12317942 A
G); (12133 12318645 A C); (12134 12318657 G A); (12135 12318687 T A); (12136 12318777 T G); (12137
12319037 C G); (12138 12319286 G A); (12139 12319417 T C); (12140 12319519 T A); (12141 12319759
G A); (12142 12319879 G A); (12143 12320379 G C); (12144 12320469 G A); (12145 12320634 C
T); (12146 12320858 A G); (12147 12320881 A G); (12148 12321083 A G); (12149 12321352 G A); (12150
12321816 C A); (12151 12322033 G A); (12152 12322474 A C); (12153 12322609 G
GACACTTTACTTA); (12154 12322922 T C); (12155 12323025 A G); (12156 12323263 G A); (12157
12323351 T C); (12158 12323701 G A); (12159 12323932 G T); (12160 12324182 G A); (12161 12324292
C CGAATTAATCGATTACAAGTGT); (12162 12324375 G A); (12163 12324393 T C); (12164 12324539 T
C); (12165 12325034 T G); (12166 12326057 C T); (12167 12327278 G A); (12168 12327397 A G); (12169
12327854 A G); (12170 12328474 G A); (12171 12328591 T G); (12172 12328658 G C); (12173 12329151
A G); (12174 12329183 C T); (12175 12329239 C T); (12176 12329266 A G); (12177 12329328 C
T); (12178 12329382 G A); (12179 12329498 C T); (12180 12329571 C T); (12181 12329861 G A); (12182
12330427 G GGCAA); (12183 12331143 GTGAGATAATGGATGTCTGAGA G); (12184 12331150 T
A); (12185 12331355 A G); (12186 12332334 C CA); (12187 12332701 C T); (12188 12332726 G A); (12189
12333089 G C); (12190 12333357 G A); (12191 12334656 T C); (12192 12334909 G C); (12193 12334910
C A); (12194 12335024 T C); (12195 12335439 G A); (12196 12335497 C G); (12197 12335773 A
G); (12198 12335958 C T); (12199 12336160 G A); (12200 12336555 T C); (12201 12336601 C T); (12202
12337276 T C); (12203 12337349 C A); (12204 12337376 G A); (12205 12337527 G A); (12206 12337575
A G); (12207 12337699 G A); (12208 12337744 G T); (12209 12337748 G T); (12210 12337957 T
C); (12211 12338006 T G); (12212 12338128 C T); (12213 12338840 T C); (12214 12339818 A G); (12215
12341541 G A); (12216 12345542 A C); (12217 12347019 G A); (12218 12347296 T C); (12219 12348312
G A); (12220 12348348 T C); (12221 12348356 T C); (12222 12348357 T A); (12223 12348367 C T); (12224
12348369 T G); (12225 12350092 T C); (12226 12350919 A G); (12227 12350933 G A); (12228 12351044
A G); (12229 12351047 A G); (12230 12351126 A G); (12231 12352027 G A); (12232 12352432 G
A); (12233 12352497 A T); (12234 12353411 T C); (12235 12353813 A T); (12236 12354258 A G); (12237
12354298 C T); (12238 12354365 C T); (12239 12354400 G A); (12240 12354478 A G); (12241 12354560
C T); (12242 12354834 C T); (12243 12354849 T C); (12244 12354941 C G); (12245 12355850 T C); (12246
12356205 C T); (12247 12356334 G A); (12248 12356419 A G); (12249 12357031 C T); (12250 12357238
C T); (12251 12357254 A G); (12252 12357554 A G); (12253 12357595 C A); (12254 12357615 G
A); (12255 12357630 G A); (12256 12357901 G A); (12257 12358013 T C); (12258 12358032 A G); (12259
12358938 A C); (12260 12358945 C T); (12261 12359526 G GTTGAGGAGCTCCGCCGTGGCA); (12262
12359888 A G); (12263 12361574 T C); (12264 12361798 A T); (12265 12362262 C T); (12266 12362452
GA G); (12267 12362525 T C); (12268 12363203 C T); (12269 12363278 G T); (12270 12363393 A
G); (12271 12363441 T C); (12272 12363456 C T); (12273 12363578 T A); (12274 12363612 A G); (12275
12363828 T C); (12276 12363989 G A); (12277 12364672 A G); (12278 12364731 G A); (12279 12365515
A G); (12280 12365813 T C); (12281 12365816 G A); (12282 12366662 G A); (12283 12367006 T
A); (12284 12367055 T C); (12285 12367263 A G); (12286 12367491 T C); (12287 12367914 C T); (12288
12367919 A C); (12289 12368089 T C); (12290 12368248 C T); (12291 12368265 C A); (12292 12368919 C
T); (12293 12368927 G A); (12294 12369026 C T); (12295 12369080 G C); (12296 12369141 G A); (12297
12369163 G T); (12298 12369232 G A); (12299 12369233 G A); (12300 12369505 T C); (12301 12369541
T C); (12302 12369754 A G); (12303 12369760 T C); (12304 12370029 A T); (12305 12370874 C T); (12306
12370895 C T); (12307 12370906 C T); (12308 12371180 T G); (12309 12371189 T C); (12310 12371458
G A); (12311 12371722 C T); (12312 12371985 C T); (12313 12372281 A G); (12314 12372310 GA
G); (12315 12372596 G A); (12316 12372742 G T); (12317 12373236 A G); (12318 12373605 G T); (12319
12373744 G A); (12320 12373753 G T); (12321 12373774 G A); (12322 12373873 G A); (12323 12373903
A C); (12324 12374060 C T); (12325 12374268 C T); (12326 12374415 C T); (12327 12374450 C T); (12328
12374528 T A); (12329 12374766 G T); (12330 12375388 A G); (12331 12375501 A G); (12332 12375735
G A); (12333 12375858 C T); (12334 12375958 T C); (12335 12376683 C T); (12336 12377128 T C); (12337
12378279 A G); (12338 12379241 A G); (12339 12379380 T C); (12340 12379997 G T); (12341 12380906
G T); (12342 12381031 G A); (12343 12381157 C G); (12344 12381203 A G); (12345 12381205 T
G); (12346 12381372 C T); (12347 12381454 C T); (12348 12381701 A G); (12349 12381717 T C); (12350
12381900 G T); (12351 12382101 C T); (12352 12382204 C T); (12353 12382263 C T); (12354 12382417
G A); (12355 12382456 C T); (12356 12382494 C A); (12357 12382610 T A); (12358 12382776 G
A); (12359 12382778 A G); (12360 12382846 A G); (12361 12383194 G A); (12362 12383195 G A); (12363
12383196 G A); (12364 12383211 A G); (12365 12383371 G C); (12366 12383477 G A); (12367 12383489
T C); (12368 12383504 A G); (12369 12383685 T A); (12370 12383869 A G); (12371 12384061 G
A); (12372 12384183 C T); (12373 12384234 A C); (12374 12384245 C T); (12375 12384434 G A); (12376
12384594 A T); (12377 12385085 A C); (12378 12385123 C T); (12379 12385255 G A); (12380 12385257
T C); (12381 12385338 G A); (12382 12385340 T C); (12383 12385482 A C); (12384 12386153 A
C); (12385 12386187 C T); (12386 12386206 G T); (12387 12386322 C A); (12388 12386593 C A); (12389
12386719 A G); (12390 12386851 T G); (12391 12387011 C T); (12392 12387148 A G); (12393 12387167
C T); (12394 12387297 G A); (12395 12387649 A C); (12396 12387655 A G); (12397 12388001 G
A); (12398 12388050 G A); (12399 12388063 G A); (12400 12388130 A G); (12401 12388537 G A); (12402
12388691 T G); (12403 12388719 A AATT); (12404 12388791 A G); (12405 12389613 T C); (12406
12389765 G T); (12407 12389776 G A); (12408 12389777 T C); (12409 12389798 G A); (12410 12389828
A G); (12411 12389829 C T); (12412 12389972 A C); (12413 12389979 T A); (12414 12390139 G
A); (12415 12390631 G T); (12416 12390811 A G); (12417 12390938 G T); (12418 12391392 C T); (12419
12391488 C G); (12420 12391612 C T); (12421 12391650 T C); (12422 12391920 A G); (12423 12391986
A G); (12424 12392006 A G); (12425 12392196 C T); (12426 12392213 A G); (12427 12393258 A
G); (12428 12393698 C T); (12429 12393705 T C); (12430 12393752 T G); (12431 12393755 A C); (12432
12393761 T G); (12433 12394002 G A); (12434 12394234 G A); (12435 12394251 G T); (12436 12394305
G C); (12437 12394667 T C); (12438 12395175 A G); (12439 12395589 A C); (12440 12395763 T
C); (12441 12396621 G A); (12442 12396655 A G); (12443 12396674 G A); (12444 12396734 C T); (12445
12397061 T A); (12446 12397315 C G); (12447 12397438 G A); (12448 12397808 G A); (12449 12397839
A G); (12450 12397864 T C); (12451 12397867 T G); (12452 12398236 T C); (12453 12399657 C T); (12454
12399663 G T); (12455 12399693 G A); (12456 12399838 T C); (12457 12400374 G A); (12458 12400574
T C); (12459 12400789 A G); (12460 12400898 G A); (12461 12400990 T C); (12462 12401111 G
A); (12463 12401295 T C); (12464 12401535 A G); (12465 12402193 T A); (12466 12402194 C T); (12467
12402280 C T); (12468 12402332 G A); (12469 12402363 C A); (12470 12402369 G C); (12471 12402431
A G); (12472 12402442 G A); (12473 12402507 G A); (12474 12402513 A G); (12475 12402533 T
C); (12476 12402699 G A); (12477 12402800 C T); (12478 12402821 T C); (12479 12402893 A G); (12480
12402957 A G); (12481 12402970 T C); (12482 12403001 C T); (12483 12403018 G A); (12484 12403040
C T); (12485 12403069 T C); (12486 12403070 T G); (12487 12403071 A C); (12488 12403109 C T); (12489
12403172 C A); (12490 12403379 G A); (12491 12403496 G T); (12492 12403547 C T); (12493 12403673
G A); (12494 12403682 A G); (12495 12403899 G A); (12496 12404555 C T); (12497 12404698 T
C); (12498 12404811 T C); (12499 12404907 C T); (12500 12404910 C T); (12501 12405099 T A); (12502
12405151 G A); (12503 12405170 A G); (12504 12405172 C T); (12505 12405573 G A); (12506 12405830
C T); (12507 12406259 G C); (12508 12406435 A C); (12509 12406445 G A); (12510 12406763 T
A); (12511 12406777 C T); (12512 12406911 T A); (12513 12407068 A G); (12514 12407186 G A); (12515
12407419 G A); (12516 12407492 T C); (12517 12407498 T C); (12518 12407533 A G); (12519 12407595
G A); (12520 12409138 A G); (12521 12410061 G A); (12522 12410233 C T); (12523 12410296 G
A); (12524 12411181 T C); (12525 12411389 A C); (12526 12411431 C T); (12527 12412014 C T); (12528
12412091 A C); (12529 12412300 G A); (12530 12412686 C T); (12531 12412725 G A); (12532 12412995
A C); (12533 12413408 T G); (12534 12413797 C T); (12535 12413975 T C); (12536 12414204 C A); (12537
12414248 G A); (12538 12415243 T C); (12539 12415346 C T); (12540 12415480 T C); (12541 12416299 T
C); (12542 12416321 AC A); (12543 12416595 T A); (12544 12416895 T C); (12545 12417326 G A); (12546
12417801 G A); (12547 12418394 C T); (12548 12418453 G A); (12549 12418985 T G); (12550 12419179
A G); (12551 12419303 T G); (12552 12419574 C CGCGCTATCGCGCGGGGTTAA); (12553 12422407 G
A); (12554 12422622 A G); (12555 12422971 C T); (12556 12423040 T G); (12557 12423170 C T); (12558
12423571 G A); (12559 12423655 G A); (12560 12423816 C T); (12561 12424098 A G); (12562 12425479
G T); (12563 12425517 G A); (12564 12426300 A G); (12565 12426429 A G); (12566 12426474 G
A); (12567 12426494 A G); (12568 12427010 T A); (12569 12427088 T G); (12570 12427098 T G); (12571
12427628 C A); (12572 12428222 A G); (12573 12428516 T C); (12574 12428534 T C); (12575 12428553
A G); (12576 12428581 G A); (12577 12428824 T C); (12578 12430053 G T); (12579 12430400 A
G); (12580 12430556 C T); (12581 12430956 G T); (12582 12431112 C T); (12583 12431235 A G); (12584
12431781 G A); (12585 12431807 C T); (12586 12431867 G A); (12587 12431923 C T); (12588 12432340
C G); (12589 12432655 C T); (12590 12432790 T C); (12591 12433448 C T); (12592 12433490 T C); (12593
12433690 A T); (12594 12433759 G A); (12595 12434002 G C); (12596 12434034 T C); (12597 12434753
G C); (12598 12434793 A G); (12599 12434903 A G); (12600 12434924 A G); (12601 12435640 C
T); (12602 12435669 T C); (12603 12435818 A C); (12604 12435930 C A); (12605 12436097 T C); (12606
12436285 T C); (12607 12436327 T C); (12608 12436468 T C); (12609 12436493 A G); (12610 12436541
A G); (12611 12436608 A C); (12612 12436609 A C); (12613 12437267 T C); (12614 12437327 T
C); (12615 12437415 A T); (12616 12437772 A G); (12617 12438092 T C); (12618 12438230 C A); (12619
12438452 A G); (12620 12438714 A G); (12621 12439305 A G); (12622 12439360 C T); (12623 12439568
A T); (12624 12440271 T C); (12625 12440575 T C); (12626 12440645 G C); (12627 12442323 C T); (12628
12442375 ATCGAT A); (12629 12442437 C T); (12630 12442481 C T); (12631 12442597 G A); (12632
12443008 C A); (12633 12443118 T A); (12634 12443172 A G); (12635 12444185 G T); (12636 12444377
A C); (12637 12444455 T G); (12638 12444560 T TAG); (12639 12444736 A AACACTATG); (12640
12445026 T G); (12641 12445491 T C); (12642 12446159 C T); (12643 12446196 T C); (12644 12447016 A
C); (12645 12447219 T C); (12646 12447590 A T); (12647 12448253 C T); (12648 12448439 A T); (12649
12448673 A T); (12650 12448689 G A); (12651 12448695 T C); (12652 12448840 C T); (12653 12449423 T
C); (12654 12449948 G A); (12655 12450497 C G); (12656 12450681 G A); (12657 12451333 T A); (12658
12451336 A G); (12659 12451382 T G); (12660 12451557 TA T); (12661 12451573 G A); (12662
12451632 A T); (12663 12451677 A G); (12664 12451725 A G); (12665 12451875 A T); (12666 12452180
GA); (12667 12452250 G T); (12668 12452308 G A); (12669 12452311 T G); (12670 12452366 T
G); (12671 12452368 GT G); (12672 12452386 G A); (12673 12452398 T G); (12674 12452399 G
A); (12675 12452506 T A); (12676 12452523 C T); (12677 12452567 T C); (12678 12452626 A G); (12679
12453018 C T); (12680 12453043 T A); (12681 12453291 G A); (12682 12453456 C T); (12683 12453478
C T); (12684 12453505 G A); (12685 12453506 T C); (12686 12453706 A G); (12687 12457227 T
A); (12688 12457346 A G); (12689 12457372 G A); (12690 12457408 T TA); (12691 12457616 A
G); (12692 12457905 A T); (12693 12457916 G T); (12694 12458180 A G); (12695 12458460 G A); (12696
12458706 G A); (12697 12458723 TA T); (12698 12458786 T A); (12699 12459571 G A); (12700
12459758 C T); (12701 12460914 C T); (12702 12461654 T G); (12703 12461780 AC A); (12704 12461784
CA C); (12705 12461964 G A); (12706 12462000 G A); (12707 12463779 TC T); (12708 12464403 C
T); (12709 12464599 CT C); (12710 12465583 A G); (12711 12466232 C A); (12712 12466655 T G); (12713
12466656 A G); (12714 12466722 G T); (12715 12467121 A G); (12716 12467259 T G); (12717 12467281
C G); (12718 12467700 A G); (12719 12467803 C T); (12720 12467833 G A); (12721 12468277 C
T); (12722 12468632 G A); (12723 12468766 A C); (12724 12468987 A T); (12725 12469166 A G); (12726
12469396 A G); (12727 12469994 T G); (12728 12470507 A G); (12729 12471193 G A); (12730 12471438
T C); (12731 12471458 TTGA T); (12732 12471485 C A); (12733 12471853 C T); (12734 12471937 T
C); (12735 12472024 T C); (12736 12472054 C A); (12737 12472079 T G); (12738 12472267 G A); (12739
12472415 T C); (12740 12472503 A G); (12741 12472595 T C); (12742 12472741 C T); (12743 12473457
A AT); (12744 12473877 T A); (12745 12473879 G A); (12746 12473903 T A); (12747 12473979 A
G); (12748 12474668 T G); (12749 12474707 T C); (12750 12474829 C G); (12751 12474840 C T); (12752
12474853 G A); (12753 12475060 A G); (12754 12476409 C G); (12755 12476622 C T); (12756 12476657
T G); (12757 12476855 A G); (12758 12476953 G A); (12759 12477548 C T); (12760 12477716 A
C); (12761 12477909 T G); (12762 12478449 G A); (12763 12479212 A G); (12764 12479362 C T); (12765
12479409 T TA); (12766 12479429 A G); (12767 12479642 C G); (12768 12479667 A G); (12769
12479730 T A); (12770 12480038 C T); (12771 12480265 G A); (12772 12480309 T G); (12773 12480525
C T); (12774 12480607 G T); (12775 12480882 T C); (12776 12481033 T C); (12777 12481131 T C); (12778
12481223 T G); (12779 12481386 A G); (12780 12481931 T G); (12781 12482066 C T); (12782 12482069
A G); (12783 12482189 A C); (12784 12482365 G A); (12785 12482366 C T); (12786 12482370 G
A); (12787 12482450 G A); (12788 12482718 C T); (12789 12482855 G A); (12790 12482909 T A); (12791
12482941 G A); (12792 12483094 T C); (12793 12483157 G T); (12794 12483196 G A); (12795 12483301
T C); (12796 12483331 C T); (12797 12483333 T C); (12798 12483341 G A); (12799 12483360 A
G); (12800 12483388 A G); (12801 12483604 A G); (12802 12483645 T G); (12803 12483792 C T); (12804
12484211 G C); (12805 12484373 A G); (12806 12484533 G A); (12807 12484626 C T); (12808 12484716
A G); (12809 12484783 C T); (12810 12485076 A G); (12811 12485119 T C); (12812 12485143 C
T); (12813 12485233 G A); (12814 12485465 G A); (12815 12485874 T C); (12816 12486141 T C); (12817
12486266 A G); (12818 12486314 T C); (12819 12486396 T C); (12820 12486457 T C); (12821 12486701 C
T); (12822 12486791 C T); (12823 12486974 A AT); (12824 12487039 G A); (12825 12487109 G A); (12826
12487126 C T); (12827 12487166 C T); (12828 12487299 C T); (12829 12487343 T C); (12830 12487656 T
C); (12831 12487709 G A); (12832 12488343 G A); (12833 12489015 C T); (12834 12489174 G T); (12835
12489294 A G); (12836 12489516 C T); (12837 12489517 A G); (12838 12489529 C G); (12839 12489550
G A); (12840 12489649 A G); (12841 12489701 T C); (12842 12489721 C T); (12843 12489761 A
G); (12844 12489762 C T); (12845 12489960 T C); (12846 12490013 G A); (12847 12490099 C G); (12848
12490149 G A); (12849 12490235 CAGG C); (12850 12490465 T A); (12851 12490601 C A); (12852
12490626 A G); (12853 12490668 T C); (12854 12490765 T C); (12855 12490841 G A); (12856 12490981
A AC); (12857 12491246 G A); (12858 12491406 T G); (12859 12491746 G A); (12860 12491769 T
C); (12861 12491922 TA T); (12862 12491945 C T); (12863 12492202 G A); (12864 12492363 A G); (12865
12492781 A C); (12866 12493525 T G); (12867 12493910 G A); (12868 12494134 G T); (12869 12494172
G A); (12870 12494288 G T); (12871 12494462 G GA); (12872 12494954 G A); (12873 12495632 T
C); (12874 12495844 T A); (12875 12496334 T A); (12876 12496366 A T); (12877 12496427 C A); (12878
12496438 A G); (12879 12496485 C T); (12880 12496529 G C); (12881 12496636 T C); (12882 12497009
G A); (12883 12497559 C G); (12884 12497799 T C); (12885 12498741 G A); (12886 12499937 GA
G); (12887 12501088 C T); (12888 12501177 C T); (12889 12502170 T C); (12890 12502505 G A); (12891
12502534 A C); (12892 12502588 G A); (12893 12502927 C T); (12894 12503033 T G); (12895 12503909
T G); (12896 12504064 C T); (12897 12504722 G A); (12898 12504984 C T); (12899 12505236 C T); (12900
12505343 G A); (12901 12505378 A G); (12902 12505429 G A); (12903 12505505 C T); (12904 12505943
T G); (12905 12506139 G A); (12906 12506988 A G); (12907 12507244 G A); (12908 12507422 C
T); (12909 12507533 A G); (12910 12507538 C T); (12911 12507614 T A); (12912 12507681 T C); (12913
12507736 G A); (12914 12507906 G A); (12915 12508184 A G); (12916 12508302 C T); (12917 12508413
A G); (12918 12508524 C G); (12919 12508733 AAGC A); (12920 12508772 T G); (12921 12509686 A
G); (12922 12509758 A G); (12923 12510609 G T); (12924 12510705 T G); (12925 12511072 C T); (12926
12511171 A T); (12927 12511345 G A); (12928 12511382 A G); (12929 12511417 G A); (12930 12511442
T C); (12931 12511462 T C); (12932 12512629 G A); (12933 12512786 T C); (12934 12512885 C T); (12935
12513203 A G); (12936 12513291 T C); (12937 12513480 A G); (12938 12513796 T A); (12939 12514035
T A); (12940 12514071 T C); (12941 12514162 A G); (12942 12514166 C T); (12943 12514525
TTATTTGCATATTGA T); (12944 12514609 G A); (12945 12514645 T C); (12946 12515008 T C); (12947
12515151 G A); (12948 12515278 A G); (12949 12515894 A G); (12950 12516057 G A); (12951 12517109
T C); (12952 12517309 G A); (12953 12517327 T A); (12954 12517882 G T); (12955 12518091 G
A); (12956 12518301 T C); (12957 12518324 A T); (12958 12518667 C T); (12959 12518928 T C); (12960
12519041 T C); (12961 12519418 C T); (12962 12519891 C A); (12963 12519924 A G); (12964 12520064
G A); (12965 12520473 A G); (12966 12520580 C A); (12967 12520766 C T); (12968 12520827 C
A); (12969 12520907 A C); (12970 12521106 C T); (12971 12521296 C T); (12972 12521803 A G); (12973
12521884 A G); (12974 12521990 T C); (12975 12522500 C T); (12976 12522788 C CT); (12977 12522906
C T); (12978 12522980 T C); (12979 12523589 T C); (12980 12523624 G A); (12981 12523852 T C); (12982
12524031 A T); (12983 12524809 C T); (12984 12524889 C T); (12985 12525320 A G); (12986 12525450
A G); (12987 12525591 T C); (12988 12525613 G A); (12989 12525669 A C); (12990 12525759 C
T); (12991 12525813 G A); (12992 12525950 G A); (12993 12526145 CCATATTGAAA C); (12994 12526434
A G); (12995 12526624 T C); (12996 12526641 A G); (12997 12526654 T C); (12998 12526735 A
G); (12999 12527272 C T); (13000 12527522 G A); (13001 12527557 G A); (13002 12527666 A G); (13003
12527708 G A); (13004 12527918 G A); (13005 12528052 T A); (13006 12528198 G A); (13007 12528593
T A); (13008 12529121 C T); (13009 12529783 G A); (13010 12529927 G C); (13011 12530091 C
T); (13012 12530668 A C); (13013 12530744 G C); (13014 12530783 G C); (13015 12531039 C T); (13016
12531094 T C); (13017 12531184 G A); (13018 12531407 C T); (13019 12531686 A T); (13020 12531729
A G); (13021 12532447 T C); (13022 12532518 T G); (13023 12532908 G T); (13024 12532974 G
A); (13025 12533054 T C); (13026 12533073 C T); (13027 12533112 A AT); (13028 12535088 C A); (13029
12536050 A G); (13030 12536213 A G); (13031 12538735 A G); (13032 12538870 TA T); (13033
12538888 AT A); (13034 12539001 C T); (13035 12539136 A C); (13036 12539537 C A); (13037 12539579
A G); (13038 12539845 A G); (13039 12539883 A G); (13040 12539991 G A); (13041 12540052 A
G); (13042 12540077 A G); (13043 12541186 C T); (13044 12541333 C T); (13045 12541874 C T); (13046
12541908 G A); (13047 12541968 A G); (13048 12541978 G A); (13049 12542038 T G); (13050 12544316
G A); (13051 12545777 A G); (13052 12549725 T C); (13053 12551985 G A); (13054 12552149 A
G); (13055 12552316 A G); (13056 12552394 G A); (13057 12552408 G T); (13058 12552672 T C); (13059
12552983 T C); (13060 12552984 T C); (13061 12552991 T C); (13062 12553151 A C); (13063 12553173 G
A); (13064 12553312 C T); (13065 12553383 T C); (13066 12553459 A G); (13067 12553791 T G); (13068
12553869 AT A); (13069 12554394 T C); (13070 12554513 C T); (13071 12554535 C G); (13072 12554760
GT G); (13073 12554783 A T); (13074 12554953 C A); (13075 12555008 T A); (13076 12555241 C
A); (13077 12556151 C T); (13078 12556181 G A); (13079 12556215 T A); (13080 12556226 T C); (13081
12556284 T C); (13082 12556890 C T); (13083 12556909 C T); (13084 12556976 C G); (13085 12557203 T
C); (13086 12557325 T C); (13087 12557378 T C); (13088 12557571 G T); (13089 12557632 A G); (13090
12557749 G A); (13091 12557769 G A); (13092 12558891 G T); (13093 12558893 G T); (13094 12558975
C A); (13095 12559228 C T); (13096 12559241 G T); (13097 12559295 C T); (13098 12559619 T G); (13099
12559905 G T); (13100 12559930 T C); (13101 12560190 T C); (13102 12560271 C G); (13103 12560309
T A); (13104 12560383 C A); (13105 12560388 T C); (13106 12560760 C A); (13107 12561046 G
A); (13108 12561571 G A); (13109 12561690 G A); (13110 12561842 A C); (13111 12561844 A C); (13112
12561846 A C); (13113 12561848 A C); (13114 12561870 G GTC); (13115 12561920 A G); (13116
12562043 T C); (13117 12562158 A G); (13118 12562359 T C); (13119 12562929 C A); (13120 12563176
T C); (13121 12566186 C T); (13122 12567345 A G); (13123 12567470 A C); (13124 12567608 A
G); (13125 12567790 A G); (13126 12567814 G A); (13127 12568040 C T); (13128 12568246 T C); (13129
12568791 G A); (13130 12568800 G A); (13131 12568841 G T); (13132 12568918 C T); (13133 12569449
G A); (13134 12569453 A G); (13135 12570129 T C); (13136 12570247 A G); (13137 12570854 A
G); (13138 12570901 T C); (13139 12571090 C T); (13140 12571343 G C); (13141 12571438 T C); (13142
12571638 C T); (13143 12571748 T C); (13144 12571899 C T); (13145 12572055 G C); (13146 12572576 A
C); (13147 12573067 T A); (13148 12573076 A G); (13149 12573149 C G); (13150 12573759 A C); (13151
12574217 A G); (13152 12574829 T G); (13153 12574980 G C); (13154 12575138 C T); (13155 12575196
A G); (13156 12575668 G GAAGGTCGAAAATGAATTA); (13157 12575888 G C); (13158 12576135 G
C); (13159 12576181 T C); (13160 12576740 C T); (13161 12577169 G A); (13162 12577970 C A); (13163
12578069 A G); (13164 12578178 C T); (13165 12578728 G A); (13166 12578959 C T); (13167 12579001
A G); (13168 12579126 C G); (13169 12579608 G C); (13170 12579780 A G); (13171 12579802 A
G); (13172 12579879 G A); (13173 12580174 A G); (13174 12580404 C A); (13175 12580420 A T); (13176
12580700 T G); (13177 12580867 A T); (13178 12581408 A G); (13179 12581499 T A); (13180 12581748
C T); (13181 12581834 G T); (13182 12581929 T C); (13183 12582074 A G); (13184 12582287 T
G); (13185 12582490 A G); (13186 12582864 T G); (13187 12583013 AT A); (13188 12583102 A
ATGTGTTTC); (13189 12583236 T A); (13190 12583278 C T); (13191 12583352 T C); (13192 12583999 T
C); (13193 12584235 A G); (13194 12584364 A G); (13195 12584501 TG T); (13196 12584574 A
G); (13197 12584957 T C); (13198 12585238 A C); (13199 12585912 C T); (13200 12585951 C T); (13201
12586856 T C); (13202 12586968 G A); (13203 12587911 C T); (13204 12588008 AT A); (13205 12588066
T C); (13206 12588680 G A); (13207 12588681 A G); (13208 12589266 A G); (13209 12589401 T
C); (13210 12589619 A C); (13211 12589675 A G); (13212 12589830 C T); (13213 12590327 T A); (13214
12590682 T TA); (13215 12591338 C T); (13216 12591380 A G); (13217 12591587 G T); (13218 12591795
G A); (13219 12591947 T G); (13220 12592065 T C); (13221 12592212 A G); (13222 12592783 T
A); (13223 12592797 A T); (13224 12593850 C G); (13225 12594112 A G); (13226 12594319 A T); (13227
12594386 G C); (13228 12594785 C T); (13229 12594923 G A); (13230 12594937 T C); (13231 12594968
A G); (13232 12595208 T C); (13233 12595254 T G); (13234 12595257 AAG A); (13235 12595684 G
A); (13236 12595804 A G); (13237 12596075 T C); (13238 12596115 G A); (13239 12596321 C T); (13240
12596541 T C); (13241 12596555 C T); (13242 12597096 T C); (13243 12597529 C T); (13244 12597545 C
T); (13245 12597855 T G); (13246 12598066 A G); (13247 12598193 C A); (13248 12598217 G A); (13249
12598442 G A); (13250 12598503 A G); (13251 12598626 A G); (13252 12598639 A C); (13253 12598726
T C); (13254 12598848 G A); (13255 12599383 G A); (13256 12600004 G A); (13257 12600163 G
A); (13258 12600489 C T); (13259 12600773 G T); (13260 12601089 T G); (13261 12601146 G A); (13262
12601336 T C); (13263 12601469 T C); (13264 12601794 T A); (13265 12602010 T A); (13266 12602188 C
A); (13267 12602806 G GA); (13268 12602874 C A); (13269 12603548 A C); (13270 12603617 G
A); (13271 12603697 A AT); (13272 12603711 G T); (13273 12603928 A G); (13274 12603988 T G); (13275
12604186 CA C); (13276 12604465 C G); (13277 12604881 A AT); (13278 12605616 T C); (13279
12606352 G A); (13280 12606596 C A); (13281 12606870 A T); (13282 12606928 G A); (13283 12607051
T C); (13284 12607146 C A); (13285 12607301 A T); (13286 12607344 G A); (13287 12607571 A
C); (13288 12607642 A G); (13289 12608405 T G); (13290 12608747 TC T); (13291 12608756 A C); (13292
12608841 C T); (13293 12609015 T A); (13294 12609102 T A); (13295 12609626 G A); (13296 12610006
T C); (13297 12610067 T C); (13298 12610095 A C); (13299 12610112 AT A); (13300 12610274 A
G); (13301 12611135 G A); (13302 12611196 T C); (13303 12611291 G A); (13304 12611558 C T); (13305
12611626 G A); (13306 12611783 A G); (13307 12611800 T A); (13308 12612165 A G); (13309 12612369
G T); (13310 12612427 A G); (13311 12612791 T C); (13312 12613013 T G); (13313 12613212 T
C); (13314 12613831 A T); (13315 12613988 C T); (13316 12615097 T C); (13317 12615108 T C); (13318
12615116 G T); (13319 12615469 T G); (13320 12615659 T A); (13321 12615685 C T); (13322 12616376
G A); (13323 12616569 C T); (13324 12616713 A G); (13325 12616728 A C); (13326 12616806 G
A); (13327 12617021 C T); (13328 12617423 C T); (13329 12617841 C T); (13330 12617843 T C); (13331
12618238 T C); (13332 12618453 C T); (13333 12618524 C T); (13334 12619028 T C); (13335 12619045 A
G); (13336 12619216 T C); (13337 12619284 C T); (13338 12619429 A G); (13339 12619431 C T); (13340
12619567 G A); (13341 12619734 C A); (13342 12619877 G A); (13343 12619989 G A); (13344 12620575
G A); (13345 12621722 G A); (13346 12621767 A T); (13347 12621787 T C); (13348 12621790 T
C); (13349 12622014 A G); (13350 12622039 A G); (13351 12622152 G A); (13352 12622905 A G); (13353
12622917 C T); (13354 12622943 T C); (13355 12622961 A G); (13356 12623302 A T); (13357 12623319 T
C); (13358 12623398 A G); (13359 12624481 G A); (13360 12624627 G T); (13361 12625220 G A); (13362
12625565 C T); (13363 12626036 A T); (13364 12626801 T C); (13365 12627005 G A); (13366 12627006 T
C); (13367 12627061 T C); (13368 12627188 C T); (13369 12627240 G A); (13370 12627245 A G); (13371
12627355 C T); (13372 12627682 A G); (13373 12627776 A G); (13374 12627813 A T); (13375 12627953
G T); (13376 12628533 T C); (13377 12628556 C T); (13378 12628557 A G); (13379 12629032 G
A); (13380 12629041 T A); (13381 12629797 C T); (13382 12629951 C T); (13383 12629998 G A); (13384
12630167 G A); (13385 12630772 G T); (13386 12631478 C T); (13387 12631796 C T); (13388 12632041
G A); (13389 12632434 C T); (13390 12632510 A G); (13391 12632667 C T); (13392 12633094 C
T); (13393 12633395 A G); (13394 12633768 A C); (13395 12633931 G A); (13396 12634448 C A); (13397
12634844 A G); (13398 12634858 T C); (13399 12634892 T C); (13400 12635043 TG T); (13401 12635064
A T); (13402 12635182 T C); (13403 12635231 G T); (13404 12635583 T C); (13405 12635648 T C); (13406
12635776 AAG A); (13407 12635803 A G); (13408 12637103 G C); (13409 12637396 CT C); (13410
12637551 C A); (13411 12638342 T C); (13412 12638656 T C); (13413 12639029 A G); (13414 12639210
C T); (13415 12639298 G T); (13416 12639537 T C); (13417 12639599 C A); (13418 12639679 C T); (13419
12639781 C T); (13420 12640041 T C); (13421 12640417 A G); (13422 12640495 A G); (13423 12640708
G A); (13424 12640719 T C); (13425 12641447 T C); (13426 12641593 G C); (13427 12641665 G
A); (13428 12641811 C T); (13429 12642874 T C); (13430 12643281 A G); (13431 12643497 G T); (13432
12643737 T C); (13433 12643745 T C); (13434 12643958 C A); (13435 12644077 T C); (13436 12644408 C
T); (13437 12644759 T C); (13438 12646655 A G); (13439 12646758 T C); (13440 12647787 C T); (13441
12648573 C T); (13442 12648712 A G); (13443 12650077 C T); (13444 12650337 A G); (13445 12650425
C T); (13446 12650707 C A); (13447 12652088 T C); (13448 12653056 G A); (13449 12655916 C
A); (13450 12656801 G T); (13451 12656859 T TTA); (13452 12656965 T C); (13453 12657217 C
T); (13454 12657304 C T); (13455 12657386 G A); (13456 12658302 T C); (13457 12658498 A G); (13458
12658499 A C); (13459 12659257 C T); (13460 12660859 A T); (13461 12660906 G A); (13462 12661188
A C); (13463 12661809 G GT); (13464 12661811 T C); (13465 12662687 G A); (13466 12666964 G
T); (13467 12667603 T C); (13468 12667711 C T); (13469 12668023 C T); (13470 12668241 G A); (13471
12668775 G A); (13472 12668798 A G); (13473 12669948 C A); (13474 12670591 C A); (13475 12670790
G A); (13476 12671222 G A); (13477 12671424 T C); (13478 12671505 G A); (13479 12671639 T
C); (13480 12671832 C T); (13481 12671993 T C); (13482 12672504 T C); (13483 12672628 A C); (13484
12672759 T C); (13485 12672766 C G); (13486 12673007 A G); (13487 12673010 T C); (13488 12673041
C T); (13489 12673090 T C); (13490 12673182 A G); (13491 12673191 G A); (13492 12673274 C
T); (13493 12673519 C T); (13494 12673642 C T); (13495 12673845 G A); (13496 12673999 T C); (13497
12674029 G A); (13498 12674048 A T); (13499 12674396 A G); (13500 12674495 G A); (13501 12674523
G A); (13502 12674525 T C); (13503 12674641 T TGA); (13504 12674668 A G); (13505 12674736 G
T); (13506 12674743 C T); (13507 12674851 G A); (13508 12674891 T A); (13509 12674935 A G); (13510
12675184 C T); (13511 12675198 A G); (13512 12675431 G C); (13513 12675444 A G); (13514 12675824
C G); (13515 12675834 G T); (13516 12676060 G A); (13517 12676612 T C); (13518 12676617 C
A); (13519 12676711 G A); (13520 12676832 A G); (13521 12677162 G A); (13522 12677164 T C); (13523
12677242 A G); (13524 12677248 A G); (13525 12677678 T C); (13526 12677703 T G); (13527 12677722
G A); (13528 12677940 G A); (13529 12678423 C T); (13530 12678437 C G); (13531 12678475 C
T); (13532 12680007 C T); (13533 12680088 C T); (13534 12680099 A G); (13535 12680452 T G); (13536
12680514 A G); (13537 12680537 T G); (13538 12680558 G T); (13539 12680561 G A); (13540 12680572
C T); (13541 12680707 A G); (13542 12681190 C T); (13543 12681224 G A); (13544 12681507 T
C); (13545 12682083 C T); (13546 12682107 G A); (13547 12682113 T C); (13548 12682216 G A); (13549
12682319 G A); (13550 12683878 C A); (13551 12684298 G A); (13552 12684360 C T); (13553 12684740
G A); (13554 12684854 A G); (13555 12684924 C G); (13556 12685033 T C); (13557 12685344 C
T); (13558 12686103 C G); (13559 12686203 A T); (13560 12686309 A G); (13561 12686315 A C); (13562
12687064 G A); (13563 12687662 G A); (13564 12687947 C T); (13565 12687990 T C); (13566 12688111
C T); (13567 12688361 T C); (13568 12688370 G GA); (13569 12688373 C T); (13570 12688464 T
C); (13571 12688473 C T); (13572 12688519 T C); (13573 12688696 G A); (13574 12688711 C G); (13575
12688793 T C); (13576 12689178 A G); (13577 12689190 A G); (13578 12689581 T C); (13579 12689594
G A); (13580 12689698 T C); (13581 12689737 G A); (13582 12690057 C T); (13583 12690072 C
T); (13584 12690146 G A); (13585 12690172 C T); (13586 12690216 T C); (13587 12690395 C T); (13588
12690514 A G); (13589 12691970 G A); (13590 12695766 T C); (13591 12695805 C T); (13592 12695831
T A); (13593 12696078 C A); (13594 12696125 T C); (13595 12696132 C T); (13596 12699226 T C); (13597
12701152 A G); (13598 12701989 T C); (13599 12702055 G A); (13600 12702190 G A); (13601 12706281
G A); (13602 12706341 G A); (13603 12706416 A G); (13604 12706560 G A); (13605 12706901 A
G); (13606 12710977 T C); (13607 12711005 G A); (13608 12711389 G A); (13609 12711594 C T); (13610
12711845 A C); (13611 12711934 A C); (13612 12712111 G A); (13613 12712522 A G); (13614 12712688
G A); (13615 12714311 C T); (13616 12714413 C G); (13617 12714485 T C); (13618 12714618 T
G); (13619 12715401 C T); (13620 12715476 T C); (13621 12715583 C G); (13622 12715601 A AG); (13623
12715779 A C); (13624 12716136 A G); (13625 12716319 T A); (13626 12716367 G A); (13627 12718100
C T); (13628 12718191 G A); (13629 12718515 TAA T); (13630 12718851 G A); (13631 12720279 T
C); (13632 12720682 A T); (13633 12720930 T A); (13634 12721135 G A); (13635 12721211 G T); (13636
12721609 C T); (13637 12721681 A G); (13638 12721997 G A); (13639 12722485 A T); (13640 12722608
A G); (13641 12723483 T C); (13642 12723546 C A); (13643 12723811 C A); (13644 12723926 G
A); (13645 12723964 G A); (13646 12723983 C T); (13647 12724233 T C); (13648 12724261 G A); (13649
12724412 G A); (13650 12724985 T G); (13651 12725276 C T); (13652 12725594 C T); (13653 12725655
G A); (13654 12725709 T C); (13655 12725717 A G); (13656 12725818 T G); (13657 12725841 G
A); (13658 12725888 C T); (13659 12725897 C T); (13660 12725899 A G); (13661 12726139 G A); (13662
12726569 G A); (13663 12726649 T C); (13664 12727040 A G); (13665 12727582 T C); (13666 12727929
G A); (13667 12728330 G T); (13668 12729417 T C); (13669 12729431 G A); (13670 12729522 G
A); (13671 12729973 C T); (13672 12730181 G A); (13673 12730265 A G); (13674 12732038 A G); (13675
12732064 C T); (13676 12732430 T C); (13677 12732451 T C); (13678 12732484 A C); (13679 12733477 A
G); (13680 12733852 T C); (13681 12733958 G A); (13682 12734016 C G); (13683 12734102 G A); (13684
12734153 G A); (13685 12734328 C T); (13686 12734509 T A); (13687 12734727 C T); (13688 12734836
G A); (13689 12735059 A G); (13690 12735439 T C); (13691 12735444 G A); (13692 12735587 A
G); (13693 12735787 T TGAGGGCTA); (13694 12735813 T G); (13695 12735874 C T); (13696 12736248 G
A); (13697 12736293 A G); (13698 12736388 G A); (13699 12736515 G A); (13700 12736536 A G); (13701
12736793 C T); (13702 12736987 A C); (13703 12737270 A G); (13704 12737303 T C); (13705 12737398
A C); (13706 12737548 T C); (13707 12737752 A G); (13708 12738063 G A); (13709 12738381 G
A); (13710 12738494 T C); (13711 12738689 G A); (13712 12738705 C A); (13713 12738849 C T); (13714
12739609 T C); (13715 12739812 C A); (13716 12739973 G C); (13717 12740507 T G); (13718 12740589
G A); (13719 12740894 T C); (13720 12741662 C A); (13721 12741909 C A); (13722 12742326 C
T); (13723 12742411 G A); (13724 12742488 G A); (13725 12742573 G A); (13726 12742622 C T); (13727
12749354 G GTT); (13728 12749824 G A); (13729 12752654 C T); (13730 12752739 A G); (13731
12752922 T C); (13732 12752945 T A); (13733 12753489 T TC); (13734 12769481 T A); (13735 12770025
T TC); (13736 12770364 G A); (13737 12772502 C T); (13738 12772610 A G); (13739 12772937 A
T); (13740 12773022 C T); (13741 12773159 A G); (13742 12773391 A G); (13743 12773452 C T); (13744
12773464 A C); (13745 12773515 T A); (13746 12773569 G C); (13747 12773612 C G); (13748 12773646
C T); (13749 12773665 C T); (13750 12774846 A G); (13751 12774992 T C); (13752 12775039 A
G); (13753 12775118 A C); (13754 12775256 C T); (13755 12776270 A G); (13756 12776457 C A); (13757
12776606 G A); (13758 12776682 C T); (13759 12776696 A T); (13760 12776945 G A); (13761 12776987
A G); (13762 12777059 G A); (13763 12777060 A G); (13764 12777068 G A); (13765 12777256 C
T); (13766 12777402 A G); (13767 12778233 A ATGCT); (13768 12778369 G A); (13769 12778394 G
A); (13770 12778462 C T); (13771 12778489 G T); (13772 12778732 C G); (13773 12778937 A C); (13774
12779130 G C); (13775 12779131 A G); (13776 12779157 C G); (13777 12779635 C T); (13778 12779751
A G); (13779 12779867 A C); (13780 12780197 A T); (13781 12780216 T G); (13782 12780244 T
C); (13783 12781083 G A); (13784 12781107 C T); (13785 12781115 G A); (13786 12781135 G A); (13787
12781195 G A); (13788 12781224 G A); (13789 12781226 A G); (13790 12781323 A G); (13791 12781346
C T); (13792 12781349 G A); (13793 12781452 T G); (13794 12781462 C T); (13795 12781590 C T); (13796
12781693 T A); (13797 12781818 T G); (13798 12782348 T G); (13799 12782488 G C); (13800 12782514
T C); (13801 12782780 A G); (13802 12782803 T C); (13803 12782914 A G); (13804 12782923 T
C); (13805 12783165 T C); (13806 12783830 G A); (13807 12783945 C T); (13808 12783984 T C); (13809
12784016 C T); (13810 12784459 T C); (13811 12784571 C T); (13812 12784772 G A); (13813 12785051 T
C); (13814 12785109 A G); (13815 12785810 C T); (13816 12786159 G A); (13817 12786557 A G); (13818
12786802 G C); (13819 12786804 G C); (13820 12786864 C G); (13821 12786971 C A); (13822 12787192
G C); (13823 12787417 A T); (13824 12787519 C T); (13825 12788384 T C); (13826 12788389 C T); (13827
12788767 G A); (13828 12789817 C T); (13829 12789977 T A); (13830 12790176 C G); (13831 12790436
T G); (13832 12790875 T C); (13833 12790886 C T); (13834 12790923 G A); (13835 12791059 T
G); (13836 12791082 T A); (13837 12791525 G C); (13838 12791650 A AT); (13839 12791902 G T); (13840
12792098 A C); (13841 12792529 T TA); (13842 12792613 T G); (13843 12792907 T C); (13844 12793039
T G); (13845 12793624 G A); (13846 12793846 G A); (13847 12793987 A G); (13848 12794140 G
A); (13849 12794360 T G); (13850 12795033 T C); (13851 12795098 C A); (13852 12795107 C T); (13853
12795750 C T); (13854 12796230 G C); (13855 12796293 C T); (13856 12796321 G A); (13857 12796344
C T); (13858 12796409 C CGCGCCATTA); (13859 12796626 C T); (13860 12796921 T A); (13861 12797050
G A); (13862 12797180 CA C); (13863 12797237 A G); (13864 12797340 T C); (13865 12797351 C
A); (13866 12797651 A G); (13867 12797912 T G); (13868 12798017 A G); (13869 12798069 T
TTA); (13870 12798556 A G); (13871 12798557 C T); (13872 12799043 A G); (13873 12799258 T
C); (13874 12799280 G T); (13875 12799414 T C); (13876 12799580 G A); (13877 12800174 A G); (13878
12800546 T C); (13879 12801291 T C); (13880 12801361 C T); (13881 12801375 C T); (13882 12801376 G
A); (13883 12801435 C T); (13884 12801441 T C); (13885 12801448 T C); (13886 12801537 G A); (13887
12801674 C T); (13888 12801702 G A); (13889 12801703 T C); (13890 12801734 T C); (13891 12802007
A T); (13892 12802030 A C); (13893 12802184 T C); (13894 12802275 G A); (13895 12802429 G
A); (13896 12802746 C T); (13897 12802785 A C); (13898 12802847 G T); (13899 12803020 G A); (13900
12803123 C G); (13901 12803124 A T); (13902 12803285 C T); (13903 12803366 C T); (13904 12803483
G A); (13905 12803532 G A); (13906 12803691 C T); (13907 12803826 G A); (13908 12803848 C
T); (13909 12804005 C T); (13910 12804067 T C); (13911 12804111 G A); (13912 12804473 C T); (13913
12804615 G T); (13914 12804798 T C); (13915 12805032 G A); (13916 12805126 G A); (13917 12805240
T C); (13918 12805430 T C); (13919 12806244 G GA); (13920 12807947 A G); (13921 12807979 G
A); (13922 12808833 G A); (13923 12809011 C T); (13924 12809024 G T); (13925 12811016 T C); (13926
12812026 T C); (13927 12812052 G A); (13928 12816163 G A); (13929 12818516 C A); (13930 12819540
T G); (13931 12819596 G A); (13932 12819685 C T); (13933 12822964 C T); (13934 12823061 G
A); (13935 12823432 A C); (13936 12823500 C T); (13937 12823726 G A); (13938 12823904 G T); (13939
12823922 C T); (13940 12824061 G T); (13941 12824080 G C); (13942 12824614 C T); (13943 12824709
G A); (13944 12824955 C G); (13945 12825006 C T); (13946 12825271 A G); (13947 12825288 T
C); (13948 12825316 G A); (13949 12825505 A G); (13950 12825584 G A); (13951 12825862 G A); (13952
12825962 A G); (13953 12825982 A G); (13954 12825993 C T); (13955 12826038 T C); (13956 12826208
G A); (13957 12826218 A C); (13958 12826271 T C); (13959 12826282 T C); (13960 12826361 A
G); (13961 12826512 T G); (13962 12826532 T A); (13963 12826748 A G); (13964 12827504 C A); (13965
12827548 TG T); (13966 12828379 T C); (13967 12828881 G A); (13968 12828981 A G); (13969
12828995 A G); (13970 12829131 C T); (13971 12829311 G A); (13972 12829378 T G); (13973 12829426
T C); (13974 12830087 G T); (13975 12830110 G T); (13976 12830488 G T); (13977 12830497 G
A); (13978 12830502 A G); (13979 12830516 A G); (13980 12830797 A G); (13981 12830854 T C); (13982
12830968 T C); (13983 12831018 A T); (13984 12831049 G A); (13985 12831088 G A); (13986 12831586
T G); (13987 12831784 T A); (13988 12832427 G T); (13989 12832925 C T); (13990 12833057 G
A); (13991 12833310 T A); (13992 12833343 A G); (13993 12833510 G A); (13994 12833551 C T); (13995
12833565 C G); (13996 12834025 A C); (13997 12834095 G A); (13998 12834099 T A); (13999 12834101
G A); (14000 12834309 G A); (14001 12835311 C G); (14002 12835446 T C); (14003 12835454 T
C); (14004 12835475 C T); (14005 12835514 C T); (14006 12835640 C T); (14007 12835871 T C); (14008
12835877 G T); (14009 12835900 A G); (14010 12836947 C T); (14011 12837982 A G); (14012 12837998
C T); (14013 12838007 T C); (14014 12838015 C A); (14015 12838032 A G); (14016 12838212 C T); (14017
12838268 A C); (14018 12838441 A T); (14019 12838458 G A); (14020 12838481 T C); (14021 12838602
A G); (14022 12838652 T A); (14023 12838691 T C); (14024 12838714 A T); (14025 12838816 C T); (14026
12839200 C A); (14027 12839209 G A); (14028 12839381 C T); (14029 12839384 T C); (14030 12839417
G A); (14031 12840415 T A); (14032 12840513 A G); (14033 12840704 A G); (14034 12840914 G
A); (14035 12840995 A G); (14036 12841014 A G); (14037 12841119 G A); (14038 12841264 C G); (14039
12841451 A C); (14040 12841937 A G); (14041 12842014 T C); (14042 12842121 A G); (14043 12842142
A G); (14044 12842199 A G); (14045 12842222 C T); (14046 12842472 T C); (14047 12842595 G
T); (14048 12842842 C T); (14049 12842857 G A); (14050 12842972 G A); (14051 12843291 G C); (14052
12843318 T C); (14053 12843710 T A); (14054 12843748 C T); (14055 12843904 A C); (14056 12843914 C
T); (14057 12844290 G T); (14058 12844856 G T); (14059 12844907 G A); (14060 12845172 C T); (14061
12845842 G A); (14062 12846015 T C); (14063 12846128 A C); (14064 12846206 G T); (14065 12846247
C T); (14066 12846265 T G); (14067 12846444 C G); (14068 12846582 G A); (14069 12846704 C
T); (14070 12846708 A AT); (14071 12846802 C T); (14072 12846875 G A); (14073 12846898 C T); (14074
12847087 A G); (14075 12848552 C G); (14076 12849416 C T); (14077 12852553 T C); (14078 12852752
G A); (14079 12853077 A C); (14080 12853135 C T); (14081 12853279 A G); (14082 12853932 G
A); (14083 12853935 C A); (14084 12854101 T C); (14085 12854563 G A); (14086 12854652 A G); (14087
12855261 C A); (14088 12855266 C T); (14089 12855280 C T); (14090 12855369 A C); (14091 12855931
G A); (14092 12855982 C T); (14093 12855987 A G); (14094 12856609 T A); (14095 12856626 G
A); (14096 12856826 C G); (14097 12856846 A G); (14098 12856862 T C); (14099 12857075 G A); (14100
12857650 T C); (14101 12857684 T C); (14102 12857964 C T); (14103 12858384 C T); (14104 12858386 C
T); (14105 12858563 T C); (14106 12858610 A G); (14107 12858816 A G); (14108 12858919 G A); (14109
12859105 T G); (14110 12859295 T C); (14111 12859332 A G); (14112 12859474 A C); (14113 12859824
A G); (14114 12860941 G A); (14115 12860999 A G); (14116 12861123 T G); (14117 12861416 A
T); (14118 12861667 T G); (14119 12862520 G C); (14120 12862527 C T); (14121 12862531 C T); (14122
12862579 T C); (14123 12862612 G T); (14124 12862967 T C); (14125 12862989 G A); (14126 12863238
A G); (14127 12864120 T C); (14128 12866835 C A); (14129 12866848 G A); (14130 12867202 G
A); (14131 12867248 A G); (14132 12867332 G A); (14133 12867784 A G); (14134 12867924 T C); (14135
12868592 C T); (14136 12868610 A C); (14137 12868673 T C); (14138 12869000 C T); (14139 12869209 C
A); (14140 12869433 T C); (14141 12869743 C G); (14142 12869807 T C); (14143 12869886 G A); (14144
12869942 A G); (14145 12870133 T G); (14146 12870402 A G); (14147 12870409 C T); (14148 12870738
G A); (14149 12870804 A C); (14150 12870960 A C); (14151 12871502 C T); (14152 12872062 G
A); (14153 12872227 T G); (14154 12872490 G A); (14155 12872614 G A); (14156 12872707 G A); (14157
12872789 C T); (14158 12873458 C T); (14159 12873507 A G); (14160 12873790 A G); (14161 12873860
T C); (14162 12874055 G A); (14163 12874136 A G); (14164 12874362 G A); (14165 12875193 AT
A); (14166 12875465 C G); (14167 12875686 G T); (14168 12875744 T A); (14169 12875836 A G); (14170
12876159 A T); (14171 12876248 A C); (14172 12876295 A C); (14173 12876319 C A); (14174 12876667
T C); (14175 12878004 A C); (14176 12878204 A G); (14177 12878637 A G); (14178 12879045 G
A); (14179 12879161 G A); (14180 12880482 T A); (14181 12880812 G A); (14182 12881064 T
TTG); (14183 12881800 A G); (14184 12881990 A AC); (14185 12881998 G A); (14186 12882327 C
T); (14187 12882942 C G); (14188 12883064 G C); (14189 12883105 G A); (14190 12883410 C T); (14191
12883592 A C); (14192 12883596 A G); (14193 12883663 T C); (14194 12884403 C T); (14195 12884446
A G); (14196 12884691 A G); (14197 12885040 A C); (14198 12885233 T G); (14199 12885374 C
T); (14200 12886825 C G); (14201 12886864 G T); (14202 12886956 A G); (14203 12887030 C T); (14204
12887291 C T); (14205 12887351 T C); (14206 12887414 G T); (14207 12887425 T G); (14208 12887526
G A); (14209 12887697 A C); (14210 12887756 C A); (14211 12888402 T C); (14212 12888964 C
T); (14213 12889071 G T); (14214 12889286 A ACC); (14215 12889594 G A); (14216 12889633 C
T); (14217 12889763 C T); (14218 12889771 C T); (14219 12889854 C T); (14220 12890137 C T); (14221
12890288 C T); (14222 12890515 A C); (14223 12890903 C T); (14224 12890992 A T); (14225 12891011 C
T); (14226 12891029 A C); (14227 12891328 T A); (14228 12891420 A G); (14229 12892008 C T); (14230
12892032 C T); (14231 12892042 T C); (14232 12892172 G A); (14233 12892491 T C); (14234 12892552 T
C); (14235 12892555 T C); (14236 12892675 G A); (14237 12892774 G A); (14238 12892800 T C); (14239
12892917 G A); (14240 12892955 C T); (14241 12892985 G A); (14242 12893084 C T); (14243 12893218
C G); (14244 12893264 A C); (14245 12893374 T C); (14246 12893637 G A); (14247 12893668 G
A); (14248 12893762 C T); (14249 12894142 A G); (14250 12894447 G A); (14251 12894484 A G); (14252
12894799 T C); (14253 12895138 T G); (14254 12895244 A G); (14255 12895274 C T); (14256 12895317
G A); (14257 12895603 A G); (14258 12895608 C T); (14259 12895613 T G); (14260 12895919 G
A); (14261 12896248 C T); (14262 12896638 C T); (14263 12896957 A G); (14264 12896959 A G); (14265
12897106 G A); (14266 12897455 G C); (14267 12897526 G A); (14268 12897667 G C); (14269 12898220
G A); (14270 12898294 C T); (14271 12898510 T C); (14272 12898570 G A); (14273 12898631 A
G); (14274 12898797 G A); (14275 12898984 C T); (14276 12899118 A T); (14277 12899140 C A); (14278
12899228 C T); (14279 12899275 T A); (14280 12899765 G A); (14281 12899884 A G); (14282 12899918
C T); (14283 12900011 G A); (14284 12900013 T C); (14285 12900079 T G); (14286 12900134 A
G); (14287 12900311 A G); (14288 12900391 A G); (14289 12900506 T G); (14290 12901027 T C); (14291
12901258 G T); (14292 12901368 T C); (14293 12901384 T C); (14294 12901519 G A); (14295 12902103
A G); (14296 12902283 C A); (14297 12902284 T C); (14298 12902345 G T); (14299 12902375 C
T); (14300 12902419 A G); (14301 12902551 C A); (14302 12902785 G A); (14303 12902850 G A); (14304
12902902 C A); (14305 12903247 C T); (14306 12903297 T C); (14307 12903559 G A); (14308 12904082
G A); (14309 12904206 C T); (14310 12904326 G A); (14311 12904641 G A); (14312 12904828 C
G); (14313 12904890 G A); (14314 12904894 G A); (14315 12904965 G A); (14316 12905231 G A); (14317
12905247 C T); (14318 12905410 T C); (14319 12905458 G A); (14320 12905535 G A); (14321 12905606
C T); (14322 12905769 G T); (14323 12906033 G A); (14324 12906043 C T); (14325 12906110 A
G); (14326 12906420 T C); (14327 12906442 A G); (14328 12906571 G A); (14329 12906695 A C); (14330
12906751 C T); (14331 12906767 G A); (14332 12906973 G C); (14333 12906995 T C); (14334 12907176
G A); (14335 12908320 A G); (14336 12908542 A G); (14337 12908652 A T); (14338 12908706 G
A); (14339 12908728 C T); (14340 12908821 T A); (14341 12908867 C T); (14342 12908999 C T); (14343
12909516 A G); (14344 12909602 T C); (14345 12909682 T C); (14346 12910039 G A); (14347 12910090
T C); (14348 12910127 T C); (14349 12910137 C T); (14350 12910698 G A); (14351 12910808 C T); (14352
12910824 C A); (14353 12911213 T A); (14354 12911954 C T); (14355 12912502 G A); (14356 12912875
G C); (14357 12912896 G A); (14358 12913064 T A); (14359 12913161 A G); (14360 12914969 T
C); (14361 12915008 T C); (14362 12915266 C T); (14363 12915643 A G); (14364 12915645 C T); (14365
12915801 C A); (14366 12915850 A G); (14367 12916593 C T); (14368 12917074 T C); (14369 12917285
A G); (14370 12917481 T C); (14371 12917502 T C); (14372 12917943 G A); (14373 12918088 A
C); (14374 12918586 T C); (14375 12921695 G A); (14376 12922601 A G); (14377 12924301 A G); (14378
12924315 T C); (14379 12924526 A G); (14380 12925240 T C); (14381 12925605 T C); (14382 12926054
G A); (14383 12926154 AT A); (14384 12926335 T G); (14385 12926694 T A); (14386 12926873 A
T); (14387 12927132 T C); (14388 12927177 A G); (14389 12927395 C T); (14390 12928612 C T); (14391
12929024 A G); (14392 12931371 A C); (14393 12931567 A G); (14394 12931708 C T); (14395 12931713
A C); (14396 12931808 G A); (14397 12931872 G A); (14398 12931895 C T); (14399 12932001 T
C); (14400 12932117 C T); (14401 12932171 C T); (14402 12932232 G A); (14403 12932387 G A); (14404
12932393 A G); (14405 12932443 C A); (14406 12932566 T C); (14407 12932778 C A); (14408 12932793
A G); (14409 12932875 C T); (14410 12932941 T C); (14411 12932995 T C); (14412 12933015 G
A); (14413 12933468 C T); (14414 12933573 C A); (14415 12933845 C T); (14416 12933917 C T); (14417
12933972 T C); (14418 12934043 T C); (14419 12934210 A G); (14420 12934575 T C); (14421 12935130
A G); (14422 12935156 G A); (14423 12935237 C T); (14424 12935335 T C); (14425 12935440 A
G); (14426 12936285 G C); (14427 12936312 T G); (14428 12936540 C T); (14429 12936928 A G); (14430
12937456 C A); (14431 12937471 T TTTTTCTCTATTTTTCTGGG); (14432 12937491 T C); (14433 12937498
T C); (14434 12937510 G A); (14435 12937523 G A); (14436 12937563 T G); (14437 12937634 G
A); (14438 12937892 T TGTAGAAGTAGGA); (14439 12937966 C T); (14440 12937988 G A); (14441
12938006 G C); (14442 12938099 G A); (14443 12939064 G A); (14444 12939158 G A); (14445 12939178
G A); (14446 12939242 G A); (14447 12940715 C T); (14448 12940882 T C); (14449 12940915 A
T); (14450 12941112 A G); (14451 12942787 G A); (14452 12943594 T C); (14453 12943851 C G); (14454
12944219 A G); (14455 12944353 A G); (14456 12944538 T C); (14457 12944734 T C); (14458 12945054
C A); (14459 12945269 A C); (14460 12945284 G A); (14461 12945315 G T); (14462 12945493 C
A); (14463 12946208 C A); (14464 12947318 C T); (14465 12947352 A T); (14466 12947476 C T); (14467
12947477 A G); (14468 12947597 T TATTAGCTTGTAATGA); (14469 12948026 C A); (14470 12948177 C
T); (14471 12948280 C T); (14472 12948302 T C); (14473 12948427 T C); (14474 12948673 T G); (14475
12948824 C T); (14476 12948963 A G); (14477 12949163 C A); (14478 12949265 A T); (14479 12951862
A G); (14480 12951888 G A); (14481 12953359 A T); (14482 12953493 T C); (14483 12953620 G
A); (14484 12953812 CT C); (14485 12953974 A G); (14486 12954090 C T); (14487 12954207 A C); (14488
12954255 T A); (14489 12954382 G A); (14490 12955087 T C); (14491 12955460 G A); (14492 12955498
G A); (14493 12955696 C A); (14494 12955954 A G); (14495 12956042 A AT); (14496 12956060 T
G); (14497 12956479 A G); (14498 12956486 G T); (14499 12957115 C T); (14500 12957489 T G); (14501
12957793 C T); (14502 12957931 T C); (14503 12958460 C T); (14504 12958957 A G); (14505 12958965 C
T); (14506 12959664 T A); (14507 12959679 A G); (14508 12959691 T C); (14509 12959717 T C); (14510
12959724 A G); (14511 12959736 G A); (14512 12959788 T A); (14513 12959822 A G); (14514 12959844
T C); (14515 12962842 A G); (14516 12963405 T C); (14517 12963512 G A); (14518 12963774 C
G); (14519 12963894 A G); (14520 12964549 G A); (14521 12965058 G T); (14522 12965139 T C); (14523
12965239 G A); (14524 12965336 A ATGATGT); (14525 12965454 C A); (14526 12965501 T A); (14527
12965514 A T); (14528 12965820 T C); (14529 12965843 G A); (14530 12965938 G A); (14531 12966029
C A); (14532 12966032 A C); (14533 12966284 G T); (14534 12966663 G A); (14535 12966771 C
T); (14536 12967349 G A); (14537 12967646 G T); (14538 12968784 A C); (14539 12982696 T A); (14540
12982698 A T); (14541 12982766 CT C); (14542 12983791 C CCAA); (14543 12984111 A G); (14544
12984921 G A); (14545 12985761 G A); (14546 12986918 T A); (14547 12986919 T A); (14548 12987071
A C); (14549 12987109 AG A); (14550 12987208 G GA); (14551 12987350 A G); (14552 12987974 A
C); (14553 12987981 T C); (14554 12988804 T C); (14555 12988825 C T); (14556 12989168 G T); (14557
12989183 T A); (14558 12989468 C T); (14559 12989617 A C); (14560 12990417 C T); (14561 12990443 C
T); (14562 12990791 T C); (14563 12990974 T C); (14564 12991006 T C); (14565 12991015 C A); (14566
12991037 A G); (14567 12991112 A G); (14568 12991186 A C); (14569 12991203 A C); (14570 12991277
C T); (14571 12991354 A G); (14572 12991382 G A); (14573 12991540 T C); (14574 12991600 T
C); (14575 12991746 G A); (14576 12991835 C T); (14577 12992134 G A); (14578 12992142 G A); (14579
12992307 G C); (14580 12992778 G T); (14581 12992812 T C); (14582 12992854 G A); (14583 12993118
T C); (14584 12993205 C T); (14585 12993418 A G); (14586 12993880 G T); (14587 12993918 G
A); (14588 12993997 T C); (14589 12994150 C T); (14590 12994240 A G); (14591 12994353 G T); (14592
12994677 G A); (14593 12995160 C T); (14594 12995429 G A); (14595 12995543 T C); (14596 12995840
T C); (14597 12995940 A G); (14598 12996508 C T); (14599 12996948 A C); (14600 13002024 A
C); (14601 13002456 G A); (14602 13003746 G A); (14603 13003849 G A); (14604 13003878 C T); (14605
13004120 C G); (14606 13004187 G A); (14607 13004260 T C); (14608 13004859 C G); (14609 13004967
C T); (14610 13005147 G A); (14611 13005300 G A); (14612 13005303 T C); (14613 13005304 A
T); (14614 13005333 C T); (14615 13005379 C A); (14616 13005526 A C); (14617 13005996 T C); (14618
13006121 T G); (14619 13006295 C T); (14620 13006312 G A); (14621 13006385 T C); (14622 13006475
C T); (14623 13006480 G A); (14624 13006673 C T); (14625 13006811 A G); (14626 13006841 C
G); (14627 13007376 G A); (14628 13007458 T C); (14629 13007753 C T); (14630 13008497 C T); (14631
13008520 A AAAACAGAC); (14632 13008551 C T); (14633 13008576 C T); (14634 13008842 A T); (14635
13008956 G A); (14636 13009334 G C); (14637 13009521 C A); (14638 13009534 G A); (14639 13009797
A G); (14640 13009865 G A); (14641 13010076 G A); (14642 13010109 C T); (14643 13010131 C
T); (14644 13010630 G C); (14645 13010786 C A); (14646 13010788 T C); (14647 13011024 C T); (14648
13011086 A G); (14649 13011213 C T); (14650 13011242 C T); (14651 13011291 C T); (14652 13011375
G A); (14653 13011596 G A); (14654 13011654 G A); (14655 13011781 G A); (14656 13011799 C
T); (14657 13011867 A G); (14658 13012237 C T); (14659 13012308 C T); (14660 13012436 A C); (14661
13012492 G A); (14662 13012659 T C); (14663 13012706 T C); (14664 13012776 T C); (14665 13012971 C
T); (14666 13013381 C T); (14667 13013415 C G); (14668 13013419 A G); (14669 13013490 C T); (14670
13013496 C G); (14671 13013646 A G); (14672 13013720 T A); (14673 13013753 G T); (14674 13013904
C T); (14675 13014035 A G); (14676 13014373 G T); (14677 13014395 G A); (14678 13014404 T
C); (14679 13014633 C T); (14680 13014640 C T); (14681 13014700 C T); (14682 13014755 A G); (14683
13014815 T C); (14684 13015325 A C); (14685 13015601 C T); (14686 13016142 A G); (14687 13016304
C G); (14688 13016526 C T); (14689 13016635 A G); (14690 13016772 T C); (14691 13016953 C
G); (14692 13017319 TATA T); (14693 13017404 A T); (14694 13017419 A T); (14695 13017745 C
T); (14696 13017791 G A); (14697 13017823 A G); (14698 13017898 T C); (14699 13018040 T G); (14700
13018254 A T); (14701 13018255 A T); (14702 13018574 G A); (14703 13019013 A T); (14704 13019304
G GA); (14705 13019470 T TA); (14706 13020269 A G); (14707 13020411 G T); (14708 13020926 T
TACCCCCC); (14709 13021077 TA T); (14710 13021206 C G); (14711 13021559 C A); (14712 13021560 A
T); (14713 13022402 T A); (14714 13022505 C T); (14715 13023042 CAAGATCTA C); (14716 13023139 G
A); (14717 13023166 T G); (14718 13024091 A G); (14719 13024093 T G); (14720 13024100 G A); (14721
13024422 T C); (14722 13024537 GGGGCC G); (14723 13024694 A G); (14724 13024708 T G); (14725
13024918 G A); (14726 13025074 G A); (14727 13025151 T C); (14728 13025364 G A); (14729 13025710
G T); (14730 13025916 G A); (14731 13026129 A T); (14732 13026264 C T); (14733 13026931 T C); (14734
13027404 A C); (14735 13030176 TA T); (14736 13032138 GA G); (14737 13032211 T A); (14738
13032348 C A); (14739 13035078 T G); (14740 13035094 T C); (14741 13035150 T G); (14742 13036599
A T); (14743 13036798 T C); (14744 13036880 C T); (14745 13036906 G A); (14746 13037027 C A); (14747
13037896 T C); (14748 13038213 G T); (14749 13038290 C G); (14750 13038421 A G); (14751 13038612
A G); (14752 13038727 A G); (14753 13039138 T C); (14754 13039220 A G); (14755 13039301 G
A); (14756 13039592 C T); (14757 13039997 C A); (14758 13040003 G A); (14759 13040042 A C); (14760
13040192 C G); (14761 13040214 T C); (14762 13040534 G A); (14763 13041062 T C); (14764 13041175
C T); (14765 13041207 A G); (14766 13041240 T C); (14767 13041704 A G); (14768 13041946 T
C); (14769 13042176 A G); (14770 13042278 A G); (14771 13042483 A G); (14772 13043007 A C); (14773
13043099 C T); (14774 13043156 A G); (14775 13043387 G A); (14776 13043566 C A); (14777 13043814
T C); (14778 13043942 T A); (14779 13044372 G C); (14780 13044526 T C); (14781 13044815 GT
G); (14782 13045265 G A); (14783 13045394 T G); (14784 13045436 G A); (14785 13045696 G A); (14786
13046512 T C); (14787 13046571 T C); (14788 13046678 A G); (14789 13046689 A G); (14790 13047170
G A); (14791 13047782 G A); (14792 13047870 C G); (14793 13048054 A C); (14794 13048186 A
G); (14795 13048699 T C); (14796 13048870 A G); (14797 13048872 C T); (14798 13048973 C T); (14799
13049018 G A); (14800 13049175 A G); (14801 13049423 G A); (14802 13049565 C A); (14803 13049656
A G); (14804 13052496 A G); (14805 13064874 C T); (14806 13064908 A T); (14807 13064936 T
C); (14808 13064942 T C); (14809 13064950 G T); (14810 13064976 C A); (14811 13065328 T C); (14812
13065386 A G); (14813 13065676 T A); (14814 13068041 C T); (14815 13068113 C T); (14816 13068357
A G); (14817 13068439 C T); (14818 13068514 G A); (14819 13068557 C A); (14820 13068576 C
T); (14821 13068670 A G); (14822 13068908 A G); (14823 13068911 T C); (14824 13069464 A G); (14825
13069924 G A); (14826 13070098 T C); (14827 13070504 G A); (14828 13070696 A G); (14829 13070724
A C); (14830 13070958 A G); (14831 13070959 C T); (14832 13071144 A G); (14833 13071384 C
CAT); (14834 13071401 G A); (14835 13071417 G A); (14836 13072294 T C); (14837 13073483 C
T); (14838 13074172 A G); (14839 13074310 C T); (14840 13074822 A G); (14841 13076056 G A); (14842
13076858 G A); (14843 13076924 A G); (14844 13076977 A G); (14845 13077152 G T); (14846 13077174
C T); (14847 13077601 G A); (14848 13077610 G A); (14849 13077718 T TTGAACAATATGACAA); (14850
13077766 A G); (14851 13078348 T TAGTTAAAA); (14852 13078537 A AC); (14853 13078723 A
T); (14854 13078725 T A); (14855 13079277 A G); (14856 13079394 A G); (14857 13079413 C T); (14858
13079417 A G); (14859 13080578 C T); (14860 13080788 C T); (14861 13080808 G A); (14862 13080829
C T); (14863 13081032 C T); (14864 13081309 C T); (14865 13081389 A G); (14866 13081416 G
A); (14867 13081473 T C); (14868 13081826 T A); (14869 13082052 C T); (14870 13082100 A G); (14871
13082281 G A); (14872 13083037 C T); (14873 13083161 A T); (14874 13083162 A T); (14875 13083466
AC A); (14876 13083524 C T); (14877 13084341 A C); (14878 13084377 C T); (14879 13084409 C
T); (14880 13084528 G GT); (14881 13084695 C T); (14882 13084962 C G); (14883 13085009 G A); (14884
13085307 C T); (14885 13085543 A G); (14886 13085544 G A); (14887 13085788 T C); (14888 13085892
G A); (14889 13086372 A T); (14890 13086678 A G); (14891 13086739 G A); (14892 13086746 A
G); (14893 13086747 A G); (14894 13086811 C T); (14895 13086924 C T); (14896 13086945 G A); (14897
13087037 A C); (14898 13087063 G T); (14899 13087626 A C); (14900 13087988 G A); (14901 13088163
T G); (14902 13088491 A T); (14903 13089056 C CT); (14904 13089531 T A); (14905 13089986 G
A); (14906 13090190 G C); (14907 13090306 C T); (14908 13090525 T G); (14909 13091003 A G); (14910
13091101 G A); (14911 13091197 A G); (14912 13091566 C A); (14913 13092018 G A); (14914 13092945
A AT); (14915 13093355 T C); (14916 13093413 G A); (14917 13093631 G A); (14918 13093670 C
T); (14919 13093677 T G); (14920 13093724 C T); (14921 13093765 A G); (14922 13094742 T C); (14923
13094801 C T); (14924 13094834 G A); (14925 13094871 C T); (14926 13094908 A G); (14927 13094915
C A); (14928 13095057 A G); (14929 13095061 T C); (14930 13095460 A T); (14931 13096096 A
G); (14932 13096130 C T); (14933 13096477 A G); (14934 13096780 T A); (14935 13096786 G A); (14936
13098969 C T); (14937 13099008 C T); (14938 13099056 C T); (14939 13099170 G A); (14940 13100328 C
T); (14941 13101200 G A); (14942 13101206 G A); (14943 13101207 A G); (14944 13103113 A G); (14945
13103120 T G); (14946 13104920 A G); (14947 13105148 G A); (14948 13105383 G A); (14949 13105427
A G); (14950 13105462 G T); (14951 13106299 G A); (14952 13106331 C T); (14953 13106419 C
T); (14954 13106449 T C); (14955 13106700 A G); (14956 13106765 G A); (14957 13106784 G T); (14958
13106899 C T); (14959 13106912 G A); (14960 13107003 A G); (14961 13107090 G A); (14962 13107264
G A); (14963 13107282 A AG); (14964 13107717 T C); (14965 13107825 C A); (14966 13107961 T
G); (14967 13108227 G A); (14968 13108508 G A); (14969 13108793 C T); (14970 13108860 TA T); (14971
13108895 A G); (14972 13108959 G C); (14973 13109225 G C); (14974 13109327 C A); (14975 13109376
T G); (14976 13109500 C A); (14977 13109597 A G); (14978 13109640 G T); (14979 13110010 T
A); (14980 13110438 C T); (14981 13110553 C T); (14982 13110570 C T); (14983 13110838 C G); (14984
13110962 A G); (14985 13111156 C T); (14986 13111200 T TG); (14987 13111454 A G); (14988
13111510 T C); (14989 13111830 C T); (14990 13111985 A G); (14991 13112236 T G); (14992 13112874
T C); (14993 13113416 A G); (14994 13113572 C T); (14995 13114741 G A); (14996 13115165 A
G); (14997 13115230 G T); (14998 13115321 T C); (14999 13115496 G A); (15000 13115966 G A); (15001
13116274 A G); (15002 13116988 G A); (15003 13117081 A G); (15004 13117085 G A); (15005 13117381
T C); (15006 13117498 TG T); (15007 13117605 A G); (15008 13117732 C T); (15009 13117846 C
T); (15010 13117874 A G); (15011 13117878 T C); (15012 13117957 C T); (15013 13117960 A G); (15014
13118018 G A); (15015 13118055 G A); (15016 13118118 T C); (15017 13118131 A G); (15018 13119248
G A); (15019 13119915 C T); (15020 13119923 A G); (15021 13120335 A G); (15022 13120635 T
A); (15023 13120680 A G); (15024 13121130 A G); (15025 13121193 A G); (15026 13121340 A G); (15027
13121461 T C); (15028 13121834 G A); (15029 13122235 T C); (15030 13122674 T C); (15031 13122797
G A); (15032 13123492 G A); (15033 13123502 G A); (15034 13123529 T C); (15035 13123681 A
G); (15036 13124006 G A); (15037 13124314 C T); (15038 13124318 G A); (15039 13124503 C T); (15040
13124877 G A); (15041 13124924 G A); (15042 13124954 T C); (15043 13125154 C T); (15044 13125364
A G); (15045 13125881 G T); (15046 13125921 A G); (15047 13126229 T C); (15048 13126618 G
A); (15049 13126863 GC G); (15050 13126902 T C); (15051 13126931 G A); (15052 13127083 G
C); (15053 13127903 G T); (15054 13127976 A C); (15055 13128562 C A); (15056 13131558 A G); (15057
13135190 G A); (15058 13135262 T C); (15059 13135446 C T); (15060 13135572 C A); (15061 13135792
A G); (15062 13135832 A G); (15063 13135861 T C); (15064 13135935 T C); (15065 13136090 T
G); (15066 13136108 T C); (15067 13136117 C T); (15068 13136247 C T); (15069 13136485 G A); (15070
13136500 G A); (15071 13136527 T C); (15072 13136827 C A); (15073 13136984 G C); (15074 13137014
C T); (15075 13137128 A G); (15076 13137151 C T); (15077 13137174 T C); (15078 13137233 G T); (15079
13137295 C T); (15080 13137392 G C); (15081 13137614 A G); (15082 13137885 A T); (15083 13138043
A T); (15084 13138103 G C); (15085 13138121 C T); (15086 13138135 T C); (15087 13138158 C T); (15088
13138479 G A); (15089 13138571 G A); (15090 13138784 A G); (15091 13139227 C T); (15092 13139414
C T); (15093 13139466 G T); (15094 13139559 C T); (15095 13140307 C T); (15096 13141849 T
TAA); (15097 13142011 T C); (15098 13142348 T C); (15099 13142405 T C); (15100 13142901 A
AATT); (15101 13143514 C CATGATT); (15102 13143697 T A); (15103 13144145 A G); (15104 13144154 A
C); (15105 13144371 C T); (15106 13144426 C T); (15107 13144700 T G); (15108 13145509 C G); (15109
13145878 C T); (15110 13145977 A C); (15111 13146086 G A); (15112 13146163 T C); (15113 13146164
T C); (15114 13146417 G C); (15115 13146614 G A); (15116 13146672 T C); (15117 13148133 A
G); (15118 13149580 C T); (15119 13149869 C T); (15120 13150006 T C); (15121 13150066 T C); (15122
13150089 G T); (15123 13150401 T C); (15124 13150462 G A); (15125 13150514 G A); (15126 13150531
C A); (15127 13150575 T C); (15128 13150739 G T); (15129 13150835 A G); (15130 13150891 T
C); (15131 13150906 T A); (15132 13150957 T C); (15133 13151001 G A); (15134 13151191 A G); (15135
13151289 G GA); (15136 13151945 T C); (15137 13151969 G T); (15138 13152029 C G); (15139
13152081 G C); (15140 13153116 A AT); (15141 13153754 T C); (15142 13153931 G T); (15143 13154181
T C); (15144 13154634 G A); (15145 13154898 C A); (15146 13155558 C T); (15147 13156470 C T); (15148
13156514 G A); (15149 13157415 A C); (15150 13157427 C A); (15151 13157447 T C); (15152 13157500
A G); (15153 13157671 C T); (15154 13158950 T G); (15155 13159430 G T); (15156 13159784 G
A); (15157 13160369 T A); (15158 13160377 G A); (15159 13160519 G C); (15160 13160534 C T); (15161
13160553 T G); (15162 13160719 G GA); (15163 13161110 C A); (15164 13161157 A C); (15165
13161442 A G); (15166 13162163 T G); (15167 13162202 A G); (15168 13162371 C T); (15169 13162515
T C); (15170 13162779 GT G); (15171 13162887 T A); (15172 13162963 G C); (15173 13163209 G
A); (15174 13163213 CTG C); (15175 13163379 A T); (15176 13163400 C G); (15177 13163402 T
C); (15178 13163410 T C); (15179 13163560 G A); (15180 13163564 A G); (15181 13163691 C T); (15182
13163967 A T); (15183 13163974 C T); (15184 13164376 C T); (15185 13164563 G A); (15186 13164946 T
C); (15187 13165151 T C); (15188 13165252 A G); (15189 13165376 C T); (15190 13165685 G A); (15191
13166070 AT A); (15192 13166343 C G); (15193 13166394 A C); (15194 13166795 A G); (15195
13167055 CT C); (15196 13167244 G A); (15197 13167258 A G); (15198 13167268 C T); (15199 13167317
T A); (15200 13167597 A AT); (15201 13167807 C T); (15202 13167836 G A); (15203 13167975 G
T); (15204 13168375 A C); (15205 13168656 G A); (15206 13169170 C T); (15207 13169497 C G); (15208
13169509 T C); (15209 13169920 G T); (15210 13170076 A G); (15211 13170330 C G); (15212 13170437
A C); (15213 13170531 C A); (15214 13170859 C T); (15215 13171212 T C); (15216 13171445 C G); (15217
13171677 G T); (15218 13171847 A AT); (15219 13172843 T C); (15220 13172884 G T); (15221 13173255
A T); (15222 13173704 T C); (15223 13174471 G C); (15224 13174477 A T); (15225 13174769 C T); (15226
13175076 C A); (15227 13175547 TA T); (15228 13175738 G T); (15229 13175752 T A); (15230 13176106
G T); (15231 13176422 A G); (15232 13176450 G A); (15233 13177672 T C); (15234 13179029 T
G); (15235 13179148 G A); (15236 13179170 A C); (15237 13179254 C T); (15238 13179453 C G); (15239
13179474 T C); (15240 13179489 T G); (15241 13179595 C T); (15242 13179747 C T); (15243 13179779 T
C); (15244 13180106 C G); (15245 13180345 G A); (15246 13180460 C T); (15247 13180707 G A); (15248
13181487 A C); (15249 13181543 C T); (15250 13181623 A G); (15251 13181718 A G); (15252 13182264
C T); (15253 13182317 G A); (15254 13182521 T C); (15255 13182812 T C); (15256 13182983 G
A); (15257 13183063 T C); (15258 13183118 AG A); (15259 13183612 G A); (15260 13183892 C
A); (15261 13184248 A G); (15262 13184463 T TA); (15263 13185466 T C); (15264 13185609 C T); (15265
13185767 A G); (15266 13185799 A G); (15267 13185848 C T); (15268 13185918 T C); (15269 13186071
C T); (15270 13186438 C T); (15271 13186610 A G); (15272 13187586 A C); (15273 13187821 A
G); (15274 13188180 A C); (15275 13188291 T A); (15276 13188432 T C); (15277 13190135 G T); (15278
13190387 C T); (15279 13192217 C T); (15280 13193647 C T); (15281 13194288 C T); (15282 13196213 G
A); (15283 13196675 A C); (15284 13198101 C T); (15285 13198162 T A); (15286 13198271 G A); (15287
13198358 G A); (15288 13199289 G A); (15289 13199349 C T); (15290 13199487 G A); (15291 13199541
G T); (15292 13199564 G A); (15293 13199700 T C); (15294 13199802 C T); (15295 13199892 A
G); (15296 13199936 C T); (15297 13199943 A C); (15298 13200026 C T); (15299 13203031 C T); (15300
13203905 A G); (15301 13203983 T TTC); (15302 13203986 T C); (15303 13204069 AG A); (15304
13204101 C T); (15305 13204274 T C); (15306 13204966 G A); (15307 13205116 G C); (15308 13205608
C T); (15309 13205727 C T); (15310 13205942 G A); (15311 13206060 CATTAT C); (15312 13206122 A
G); (15313 13206309 T C); (15314 13206624 T C); (15315 13207289 A C); (15316 13207369 T C); (15317
13207665 A G); (15318 13208346 G A); (15319 13208408 A G); (15320 13208409 A G); (15321 13208641
T C); (15322 13209045 A G); (15323 13209173 C A); (15324 13209559 G A); (15325 13209612 G
A); (15326 13209725 G A); (15327 13210088 C T); (15328 13210102 C G); (15329 13210106 A T); (15330
13210142 A G); (15331 13210375 A G); (15332 13211722 G A); (15333 13211939 T G); (15334 13211994
G A); (15335 13212039 G C); (15336 13212123 T C); (15337 13212167 T G); (15338 13212309 G
A); (15339 13212322 G A); (15340 13212434 T G); (15341 13212603 G A); (15342 13212789 C T); (15343
13212885 A C); (15344 13212901 G A); (15345 13212913 T G); (15346 13213087 T C); (15347 13214495
G A); (15348 13214614 T C); (15349 13214660 G A); (15350 13214984 G A); (15351 13215772 G
A); (15352 13215840 G T); (15353 13216062 TGAGG T); (15354 13216087 A T); (15355 13216088 G
A); (15356 13216100 A G); (15357 13216232 C A); (15358 13216844 C T); (15359 13217393 C T); (15360
13217405 T A); (15361 13217655 A T); (15362 13217733 A G); (15363 13217835 A G); (15364 13217847
T C); (15365 13217865 C T); (15366 13217926 T C); (15367 13218287 T G); (15368 13218377 C A); (15369
13218439 G A); (15370 13218593 G C); (15371 13218625 A T); (15372 13218632 A G); (15373 13218763
T C); (15374 13218768 C G); (15375 13218948 C T); (15376 13219361 T G); (15377 13219381 T C); (15378
13219787 T G); (15379 13219925 G A); (15380 13220032 T C); (15381 13220041 A G); (15382 13220658
A G); (15383 13221364 G C); (15384 13221473 G A); (15385 13221642 C T); (15386 13221732 C
T); (15387 13222183 C T); (15388 13222202 G A); (15389 13222771 A G); (15390 13223115 A G); (15391
13223186 C T); (15392 13223276 A G); (15393 13223341 C A); (15394 13223437 C T); (15395 13223669
T C); (15396 13223776 A G); (15397 13223837 G A); (15398 13224272 A G); (15399 13224324 G
A); (15400 13224359 G A); (15401 13224434 G A); (15402 13224455 T C); (15403 13224527 C G); (15404
13224557 G A); (15405 13224636 T C); (15406 13224754 T C); (15407 13225024 T C); (15408 13225108 C
A); (15409 13225118 T C); (15410 13225212 T C); (15411 13225288 C T); (15412 13225368 T C); (15413
13225746 C G); (15414 13225981 A G); (15415 13226129 C T); (15416 13226273 T G); (15417 13226366
G A); (15418 13226440 T C); (15419 13226720 CT C); (15420 13226813 G A); (15421 13226896 A
G); (15422 13227035 A G); (15423 13227480 C T); (15424 13227717 G A); (15425 13228350 G T); (15426
13228443 A G); (15427 13228463 T G); (15428 13228466 A G); (15429 13228494 G A); (15430 13228501
G A); (15431 13228636 A G); (15432 13228686 T A); (15433 13229475 A C); (15434 13229928 G
A); (15435 13229938 C T); (15436 13229986 A T); (15437 13230776 T C); (15438 13230784 T C); (15439
13231130 C T); (15440 13231574 A G); (15441 13231598 C T); (15442 13231756 C G); (15443 13231847
A G); (15444 13232001 T C); (15445 13232394 T C); (15446 13232736 G T); (15447 13232763 G
A); (15448 13232784 C G); (15449 13233139 C T); (15450 13233372 C T); (15451 13233556 A C); (15452
13234379 G A); (15453 13234565 T C); (15454 13234810 T C); (15455 13234824 G A); (15456 13235058
C T); (15457 13235154 G A); (15458 13235372 C T); (15459 13235952 C T); (15460 13236410 A
G); (15461 13236555 T G); (15462 13236643 G A); (15463 13236681 A G); (15464 13236737 G A); (15465
13236979 G A); (15466 13238544 G A); (15467 13238640 C T); (15468 13238708 A G); (15469 13238797
AAAGT A); (15470 13239102 T C); (15471 13239105 C T); (15472 13239644 A G); (15473 13239777 C
T); (15474 13240302 T G); (15475 13240716 A G); (15476 13241293 A G); (15477 13242270 TTG
T); (15478 13242727 C T); (15479 13242938 GA G); (15480 13243272 T C); (15481 13243403 G A); (15482
13243712 G A); (15483 13243887 A G); (15484 13244108 C T); (15485 13244268 C T); (15486 13244395
G C); (15487 13244526 TA T); (15488 13244559 C T); (15489 13244596 G A); (15490 13244648 A
G); (15491 13244947 A G); (15492 13244958 C T); (15493 13245119 A G); (15494 13245135 G A); (15495
13245194 T G); (15496 13245251 G C); (15497 13246049 A G); (15498 13246121 TG T); (15499
13246467 A G); (15500 13247559 A G); (15501 13247588 G A); (15502 13248005 C T); (15503 13248161
C T); (15504 13248346 G A); (15505 13248657 G A); (15506 13248900 A C); (15507 13249664 C
T); (15508 13249728 G A); (15509 13249914 G A); (15510 13250015 C T); (15511 13250027 C T); (15512
13250823 A G); (15513 13250839 C T); (15514 13251164 T C); (15515 13251364 T C); (15516 13251390 T
C); (15517 13251523 G T); (15518 13251814 A G); (15519 13251880 T C); (15520 13251919 T C); (15521
13252760 A C); (15522 13252811 A G); (15523 13252860 A G); (15524 13252935 G C); (15525 13253158
T C); (15526 13253295 G A); (15527 13253441 T C); (15528 13253771 A G); (15529 13253797 G
T); (15530 13253798 T A); (15531 13254054 T C); (15532 13254287 G A); (15533 13255146 T G); (15534
13255177 G A); (15535 13255607 T C); (15536 13255787 G A); (15537 13256182 T C); (15538 13256498
A C); (15539 13257176 C A); (15540 13257373 C A); (15541 13257597 A G); (15542 13257905 G
A); (15543 13258071 C T); (15544 13258140 C T); (15545 13258669 T C); (15546 13258782 T C); (15547
13258826 GT G); (15548 13259026 C T); (15549 13259435 T C); (15550 13259825 C G); (15551 13259965
A T); (15552 13260049 C A); (15553 13260775 GT G); (15554 13260874 C T); (15555 13261213 T
TATGTGGCAGATGCAAGTG); (15556 13261424 A G); (15557 13261686 G A); (15558 13261917 A
G); (15559 13261988 A T); (15560 13262217 C T); (15561 13262454 G T); (15562 13263517 C T); (15563
13264078 T A); (15564 13264403 C A); (15565 13264519 A G); (15566 13264791 A G); (15567 13264834
TC T); (15568 13265174 A C); (15569 13265823 C T); (15570 13266134 A G); (15571 13266160 CT
C); (15572 13266275 G T); (15573 13266732 A G); (15574 13266963 C T); (15575 13266974 C T); (15576
13267138 T A); (15577 13267216 A G); (15578 13267236 C T); (15579 13267460 G A); (15580 13267616
T C); (15581 13268093 T C); (15582 13268121 A G); (15583 13268188 G T); (15584 13268346 T
G); (15585 13268410 T TGA); (15586 13268536 C A); (15587 13269033 G T); (15588 13269083 C
A); (15589 13270208 A G); (15590 13270483 C T); (15591 13270606 G A); (15592 13270635 C T); (15593
13270678 G A); (15594 13271229 C T); (15595 13271416 A G); (15596 13271449 A T); (15597 13271946
T TA); (15598 13272075 A C); (15599 13272191 G T); (15600 13272227 T C); (15601 13272381 T
C); (15602 13272529 G A); (15603 13272846 C G); (15604 13273175 A G); (15605 13273346 A T); (15606
13273371 T C); (15607 13273403 C T); (15608 13273710 C A); (15609 13273812 G C); (15610 13273871 T
C); (15611 13274036 C G); (15612 13274292 A T); (15613 13274300 T C); (15614 13274366 A G); (15615
13274414 G A); (15616 13274486 T C); (15617 13274529 G A); (15618 13274632 T C); (15619 13274648
T C); (15620 13274845 G T); (15621 13274962 T C); (15622 13275060 G A); (15623 13275062 A
G); (15624 13275391 A G); (15625 13275615 A C); (15626 13276011 C CA); (15627 13276175 T C); (15628
13276650 C T); (15629 13277508 T A); (15630 13277509 A C); (15631 13277549 C T); (15632 13277578
G A); (15633 13278278 G A); (15634 13278518 T C); (15635 13278641 C CT); (15636 13278837 T
C); (15637 13278860 C T); (15638 13279245 C A); (15639 13279318 C T); (15640 13280072 C T); (15641
13280677 A T); (15642 13280730 T A); (15643 13280732 C T); (15644 13280735 C T); (15645 13281014 T
C); (15646 13281056 C T); (15647 13281396 T C); (15648 13281465 C G); (15649 13281547 A G); (15650
13281597 G C); (15651 13281647 G A); (15652 13281707 C T); (15653 13281950 G A); (15654 13282019
T C); (15655 13282142 G T); (15656 13282188 A G); (15657 13282332 C A); (15658 13282502 A
C); (15659 13283454 T C); (15660 13284609 A ACCTAAAACTAG); (15661 13284868 T C); (15662
13284870 C T); (15663 13284879 C A); (15664 13284892 G A); (15665 13284899 G A); (15666 13285074
C T); (15667 13285211 C T); (15668 13285261 T C); (15669 13285275 C T); (15670 13285488 C G); (15671
13285526 G A); (15672 13285752 C T); (15673 13285763 A C); (15674 13285787 G A); (15675 13285825
G A); (15676 13285855 T C); (15677 13286097 G A); (15678 13286557 GA AG); (15679 13287698 C
T); (15680 13287705 G A); (15681 13287826 G A); (15682 13287866 G A); (15683 13288694 C A); (15684
13288784 A G); (15685 13289090 T G); (15686 13289095 G C); (15687 13289636 A G); (15688 13290091
T A); (15689 13290131 C G); (15690 13290301 C T); (15691 13290553 A AT); (15692 13290682 A
G); (15693 13291106 G A); (15694 13291123 G C); (15695 13291841 T C); (15696 13292471 G A); (15697
13292503 A G); (15698 13292521 G A); (15699 13292753 T C); (15700 13292843 A C); (15701 13292891
C T); (15702 13292916 T TA); (15703 13293197 C A); (15704 13293200 C T); (15705 13293327 C
T); (15706 13293402 C T); (15707 13293513 T C); (15708 13293594 C T); (15709 13294168 C T); (15710
13294592 C T); (15711 13295385 C T); (15712 13297301 G A); (15713 13297603 G A); (15714 13297632
C T); (15715 13298307 A G); (15716 13298319 A G); (15717 13298356 C T); (15718 13298371 A
G); (15719 13298373 T C); (15720 13298376 G A); (15721 13298409 C T); (15722 13298620 C T); (15723
13298798 T C); (15724 13300391 A C); (15725 13300394 C T); (15726 13300892 T C); (15727 13300899 G
T); (15728 13300974 C T); (15729 13302159 A G); (15730 13302442 C T); (15731 13302620 T C); (15732
13302659 T C); (15733 13302785 G T); (15734 13302809 C A); (15735 13302858 G A); (15736 13302919
A T); (15737 13302952 C A); (15738 13303001 G A); (15739 13303021 C G); (15740 13303072 C
T); (15741 13303092 A G); (15742 13303262 A T); (15743 13303691 T G); (15744 13304574 A G); (15745
13304583 C T); (15746 13304944 T G); (15747 13304949 C A); (15748 13305153 A AT); (15749 13305198
G A); (15750 13305204 A T); (15751 13305232 T C); (15752 13305473 C T); (15753 13305596 G
GT); (15754 13305610 C G); (15755 13306247 C T); (15756 13306290 T C); (15757 13306504 C T); (15758
13306722 A AT); (15759 13307396 G T); (15760 13308000 G A); (15761 13308081 T C); (15762 13308645
C T); (15763 13309563 A G); (15764 13310188 A G); (15765 13310229 G A); (15766 13310350 T
C); (15767 13310631 G A); (15768 13310641 A T); (15769 13310865 A G); (15770 13311204 C T); (15771
13311665 G C); (15772 13311707 T C); (15773 13311762 A AT); (15774 13311881 G A); (15775
13312073 G A); (15776 13312183 C T); (15777 13312239 G A); (15778 13312240 A T); (15779 13312241
T C); (15780 13312311 T C); (15781 13312404 C T); (15782 13313637 C A); (15783 13314158 T C); (15784
13314210 C T); (15785 13314496 T C); (15786 13314723 G T); (15787 13315702 G A); (15788 13315887
G A); (15789 13315924 G C); (15790 13316318 C T); (15791 13316418 A G); (15792 13316519 T
C); (15793 13316591 G T); (15794 13316890 G A); (15795 13317298 A G); (15796 13317300 C T); (15797
13317454 G A); (15798 13317515 A G); (15799 13317606 G A); (15800 13317771 G T); (15801 13317817
C T); (15802 13317944 T C); (15803 13318160 G C); (15804 13318173 T C); (15805 13319211 G T); (15806
13319289 G A); (15807 13319712 G A); (15808 13319884 G GA); (15809 13319973 C T); (15810
13320048 C A); (15811 13320098 T C); (15812 13320275 G T); (15813 13320373 T G); (15814 13320414
T C); (15815 13320529 G A); (15816 13320530 G C); (15817 13320621 C T); (15818 13320979 C T); (15819
13320992 A T); (15820 13321527 T C); (15821 13321592 C T); (15822 13321620 A G); (15823 13322098
G A); (15824 13322162 C T); (15825 13322473 A G); (15826 13322635 G A); (15827 13322728 T
C); (15828 13322798 TTGGTCAACCATC T); (15829 13323124 C T); (15830 13323365 G A); (15831
13323744 G A); (15832 13323939 C A); (15833 13323979 C T); (15834 13324054 C T); (15835 13324102
G A); (15836 13324179 T C); (15837 13325195 C T); (15838 13325551 T C); (15839 13326045 C T); (15840
13326207 C T); (15841 13327277 C A); (15842 13327594 T C); (15843 13327905 T C); (15844 13328092 T
C); (15845 13328446 C T); (15846 13328464 C T); (15847 13328670 T C); (15848 13328867 T G); (15849
13329050 T C); (15850 13329140 C G); (15851 13329367 A C); (15852 13329793 C G); (15853 13329970
C T); (15854 13329981 A G); (15855 13330061 C T); (15856 13330065 T A); (15857 13330399 G
A); (15858 13330567 C T); (15859 13330758 C T); (15860 13330826 A G); (15861 13331059 T TA); (15862
13331141 T G); (15863 13331503 A G); (15864 13331631 G A); (15865 13331717 T G); (15866 13331727
C T); (15867 13332025 A G); (15868 13332820 AT A); (15869 13332901 G A); (15870 13333346 AG
A); (15871 13333490 A C); (15872 13333681 A G); (15873 13334390 T TA); (15874 13335078
TGGCGCCATC T); (15875 13337480 T C); (15876 13338064 G A); (15877 13339072 AAT A); (15878
13339673 A AT); (15879 13340205 G T); (15880 13340288 T G); (15881 13340810 T A); (15882 13340847
T A); (15883 13340905 C CAT); (15884 13341678 A G); (15885 13341704 G C); (15886 13342265 G
A); (15887 13342438 A C); (15888 13342509 A C); (15889 13343062 A G); (15890 13343639 T C); (15891
13344154 C G); (15892 13344260 G A); (15893 13344842 A G); (15894 13345502 C T); (15895 13345506
G A); (15896 13345552 T C); (15897 13346084 T C); (15898 13346100 G T); (15899 13346127 G
A); (15900 13346128 T C); (15901 13346172 T A); (15902 13346348 C CA); (15903 13347047 G T); (15904
13347161 A G); (15905 13347373 G GT); (15906 13347927 C T); (15907 13348243 T C); (15908 13348318
A G); (15909 13348345 C T); (15910 13348451 T C); (15911 13348472 G T); (15912 13348539 C T); (15913
13348542 A C); (15914 13348673 T C); (15915 13349060 C T); (15916 13349281 C T); (15917 13349532 C
T); (15918 13349608 A G); (15919 13349654 C T); (15920 13349670 C T); (15921 13349696 C T); (15922
13349851 A G); (15923 13350045 T C); (15924 13350100 A C); (15925 13350280 A G); (15926 13350776
GT G); (15927 13350972 C T); (15928 13351036 T C); (15929 13351260 A T); (15930 13351398 C
T); (15931 13351477 AAGT A); (15932 13351478 C A); (15933 13351479 A C); (15934 13353018 T
C); (15935 13353025 C A); (15936 13354260 C T); (15937 13354352 A AT); (15938 13354516 G A); (15939
13355668 T C); (15940 13355755 T C); (15941 13356068 A C); (15942 13356241 G T); (15943 13356250 C
T); (15944 13356286 A G); (15945 13356304 G T); (15946 13356478 C T); (15947 13356513 G A); (15948
13356637 G A); (15949 13356695 G C); (15950 13356834 C T); (15951 13356938 T C); (15952 13357074
A AT); (15953 13357177 C T); (15954 13357363 C T); (15955 13357370 C T); (15956 13357536 C
T); (15957 13357627 A T); (15958 13357774 A G); (15959 13358070 T C); (15960 13358075 G A); (15961
13358093 C T); (15962 13358199 C T); (15963 13358223 C T); (15964 13358322 C T); (15965 13358768 C
T); (15966 13358769 T C); (15967 13358770 A C); (15968 13359036 T C); (15969 13359037 C T); (15970
13359044 C T); (15971 13359109 T G); (15972 13359127 T A); (15973 13359281 G A); (15974 13359661
A G); (15975 13359738 G A); (15976 13359791 C T); (15977 13359844 G A); (15978 13360001 C
T); (15979 13360048 C T); (15980 13360136 T C); (15981 13360304 G C); (15982 13360620 A G); (15983
13361525 T A); (15984 13361741 C A); (15985 13361742 T A); (15986 13362174 A G); (15987 13362284
C T); (15988 13362459 C T); (15989 13362614 A G); (15990 13362706 CT C); (15991 13362898 T
C); (15992 13363160 G GTA); (15993 13363242 G A); (15994 13363430 G A); (15995 13363607 T
A); (15996 13363825 C T); (15997 13365143 T A); (15998 13365356 C T); (15999 13365499 A G); (16000
13365957 T C); (16001 13366186 G A); (16002 13366358 A T); (16003 13368253 C G); (16004 13368291
G A); (16005 13368370 G A); (16006 13368437 C T); (16007 13368519 G A); (16008 13368530 A
G); (16009 13368791 G GA); (16010 13368904 G A); (16011 13369065 A G); (16012 13369525 T
C); (16013 13370371 T C); (16014 13370418 C T); (16015 13370621 G T); (16016 13370827 TA T); (16017
13370992 T A); (16018 13371485 T C); (16019 13371707 A G); (16020 13371717 T C); (16021 13371981
A G); (16022 13373353 A G); (16023 13373472 T C); (16024 13373604 G A); (16025 13373626 G
T); (16026 13374557 G T); (16027 13374664 C G); (16028 13374738 A T); (16029 13374977 G A); (16030
13375018 C T); (16031 13375175 C T); (16032 13375690 A G); (16033 13375729 C A); (16034 13375830
A G); (16035 13376489 C A); (16036 13376822 C T); (16037 13377589 G A); (16038 13378089 A
C); (16039 13378718 G C); (16040 13378932 C T); (16041 13378994 C T); (16042 13379042 A C); (16043
13379045 T C); (16044 13379055 C T); (16045 13379453 C T); (16046 13379461 A G); (16047 13379750
A T); (16048 13379839 A G); (16049 13380154 C T); (16050 13380244 G A); (16051 13380552
CAACAAAAACAGATAGAATTAAACA C); (16052 13380602 T C); (16053 13380717 C T); (16054 13380752
A T); (16055 13381034 T C); (16056 13381245 T G); (16057 13381551 A G); (16058 13381732 C
G); (16059 13381787 C T); (16060 13381965 T C); (16061 13382555 T C); (16062 13383029 A G); (16063
13383094 T C); (16064 13383362 T A); (16065 13384223 C CTA); (16066 13384312 C T); (16067
13384625 C G); (16068 13384986 A T); (16069 13385044 C T); (16070 13385427 T C); (16071 13385492
G A); (16072 13385837 C T); (16073 13386663 G A); (16074 13386843 A G); (16075 13386964 C
G); (16076 13387558 T C); (16077 13387590 G T); (16078 13388089 G A); (16079 13388112 G A); (16080
13388161 T G); (16081 13388796 C A); (16082 13389150 C A); (16083 13389785 A C); (16084 13389876
A C); (16085 13389938 G A); (16086 13390162 C T); (16087 13390485 T C); (16088 13390654 T C); (16089
13390671 A G); (16090 13390938 G A); (16091 13391187 A G); (16092 13391549 T A); (16093 13391714
T TA); (16094 13391741 A G); (16095 13391906 A G); (16096 13392093 G A); (16097 13392295 A
C); (16098 13392403 T C); (16099 13392431 T C); (16100 13392940 T C); (16101 13393075 T C); (16102
13393302 G A); (16103 13393352 G A); (16104 13393375 T C); (16105 13393435 A G); (16106 13394508
C T); (16107 13394724 A G); (16108 13394791 G T); (16109 13394792 T A); (16110 13394883 A
C); (16111 13395027 G A); (16112 13395242 C T); (16113 13395290 C T); (16114 13395871 T C); (16115
13396077 CT C); (16116 13396260 C T); (16117 13396471 A C); (16118 13396636 T A); (16119 13396676
CAT C); (16120 13396723 A G); (16121 13396809 A G); (16122 13396980 G A); (16123 13397310 C
T); (16124 13397420 G A); (16125 13397437 T C); (16126 13397506 A G); (16127 13397589 G C); (16128
13397591 A T); (16129 13397772 A G); (16130 13397890 T A); (16131 13398795 C T); (16132 13398859
C T); (16133 13398989 C T); (16134 13399109 A G); (16135 13399119 A G); (16136 13399140 C
T); (16137 13399179 A C); (16138 13399277 C T); (16139 13399286 T C); (16140 13399427 T A); (16141
13399439 A T); (16142 13399927 C T); (16143 13399958 C T); (16144 13400480 G A); (16145 13401305
G A); (16146 13401430 C T); (16147 13401788 G C); (16148 13401797 G C); (16149 13401812 C
T); (16150 13401836 T G); (16151 13402126 T C); (16152 13402145 ATTGTT A); (16153 13402178 C
T); (16154 13403643 A AG); (16155 13403997 CT C); (16156 13404323 T G); (16157 13404338 T
A); (16158 13404885 T A); (16159 13406078 G A); (16160 13406592 G C); (16161 13406806 T A); (16162
13407005 T G); (16163 13407084 T A); (16164 13407290 C T); (16165 13407675 G C); (16166 13408395
T C); (16167 13408827 G C); (16168 13409293 T C); (16169 13409727 A T); (16170 13409999 G
A); (16171 13410053 G A); (16172 13410080 G A); (16173 13410088 A G); (16174 13410110 A
AT); (16175 13410182 A T); (16176 13410236 A AG); (16177 13410323 A G); (16178 13410489 T
C); (16179 13410632 C T); (16180 13410685 C A); (16181 13410690 C T); (16182 13410737 T C); (16183
13411521 T C); (16184 13411641 A G); (16185 13411740 C T); (16186 13411882 C G); (16187 13412028
C T); (16188 13412319 CGATTT C); (16189 13412686 A G); (16190 13412809 T C); (16191 13412993 G
A); (16192 13413126 T C); (16193 13413545 G A); (16194 13413601 T C); (16195 13414073 C T); (16196
13414357 T A); (16197 13414358 T A); (16198 13414471 A G); (16199 13414608 A C); (16200 13414619
C T); (16201 13414693 A G); (16202 13414753 C T); (16203 13414762 T C); (16204 13415202 A
G); (16205 13415263 T G); (16206 13416421 T C); (16207 13416501 TC T); (16208 13416865 C G); (16209
13417917 A ACAGG); (16210 13418100 C A); (16211 13418117 G A); (16212 13419574 C T); (16213
13419723 C T); (16214 13419813 C T); (16215 13419849 A G); (16216 13419861 T TA); (16217 13419941
A T); (16218 13420163 AT A); (16219 13420307 C A); (16220 13420375 A AT); (16221 13420449 T
C); (16222 13421157 C T); (16223 13421465 T C); (16224 13421716 G T); (16225 13421968 A G); (16226
13422062 A G); (16227 13422590 C T); (16228 13423195 A G); (16229 13423289 T G); (16230 13423295
C T); (16231 13423779 A C); (16232 13423907 G A); (16233 13423962 G A); (16234 13423974 G
A); (16235 13424525 A G); (16236 13424945 C A); (16237 13425209 G A); (16238 13425678 T C); (16239
13425713 T TA); (16240 13426315 T C); (16241 13426412 T C); (16242 13426750 G C); (16243 13426812
T C); (16244 13426885 A G); (16245 13426908 C T); (16246 13426936 T C); (16247 13427630 T C); (16248
13428056 C T); (16249 13428524 T A); (16250 13428678 C A); (16251 13429960 T C); (16252 13430030
G A); (16253 13430777 A G); (16254 13430904 CAGGCCCAAGAAGCACCA C); (16255 13431256 C
T); (16256 13431463 TC T); (16257 13431494 A G); (16258 13431522 G A); (16259 13432114 G T); (16260
13432183 CT C); (16261 13432222 T G); (16262 13432315 A C); (16263 13432356 G A); (16264 13432426
C T); (16265 13433390 A G); (16266 13433709 C G); (16267 13433720 C T); (16268 13434907 C
A); (16269 13435151 T C); (16270 13435539 A G); (16271 13435651 G A); (16272 13435658 T C); (16273
13435680 G T); (16274 13435728 A G); (16275 13435897 T C); (16276 13436026 C T); (16277 13436102
A G); (16278 13436151 C T); (16279 13437038 C A); (16280 13437379 G A); (16281 13438054 G
C); (16282 13438185 T G); (16283 13438305 C T); (16284 13438343 A G); (16285 13439085 G A); (16286
13439130 G T); (16287 13440047 C G); (16288 13440161 G A); (16289 13440301 A C); (16290 13440766
G A); (16291 13441165 G A); (16292 13441589 C T); (16293 13441630 T C); (16294 13441681 A
G); (16295 13441755 C T); (16296 13441773 T G); (16297 13441966 G A); (16298 13442247 T G); (16299
13442326 C A); (16300 13442398 C T); (16301 13442863 T A); (16302 13443782 AC A); (16303 13443861
A G); (16304 13444235 C T); (16305 13444640 G A); (16306 13444782 T C); (16307 13445264 A
T); (16308 13445328 C T); (16309 13445374 C T); (16310 13445432 T C); (16311 13445468 T C); (16312
13445650 G A); (16313 13445924 A G); (16314 13445966 C T); (16315 13446061 C T); (16316 13446226
T A); (16317 13447184 T C); (16318 13447256 T C); (16319 13447305 G A); (16320 13447638 T A); (16321
13447755 G A); (16322 13447894 G A); (16323 13448923 A G); (16324 13449598 G
GATGTTGGAT); (16325 13449684 T C); (16326 13449907 A G); (16327 13451004 A G); (16328 13451198
A T); (16329 13451370 C A); (16330 13451804 G C); (16331 13452098 T C); (16332 13452721 T
G); (16333 13453006 GGT G); (16334 13453012 A G); (16335 13453228 C T); (16336 13453472 T
A); (16337 13453546 A T); (16338 13453715 C T); (16339 13453793 C T); (16340 13454384 A C); (16341
13454620 A G); (16342 13454860 G T); (16343 13454941 AAAAGTTT A); (16344 13455166 G T); (16345
13455221 G A); (16346 13455793 C T); (16347 13455818 T G); (16348 13456500 G A); (16349 13456558
C T); (16350 13457017 A G); (16351 13457136 C T); (16352 13457938 A G); (16353 13458033 AC
A); (16354 13458247 C T); (16355 13458308 T C); (16356 13458473 A G); (16357 13458610 C T); (16358
13458751 G A); (16359 13459257 G A); (16360 13459294 A G); (16361 13459317 G A); (16362 13459510
T C); (16363 13459555 C T); (16364 13459591 A C); (16365 13459595 C T); (16366 13459655 G A); (16367
13460264 T G); (16368 13460301 T TG); (16369 13460667 G T); (16370 13460694 A T); (16371 13460872
G A); (16372 13462280 GT G); (16373 13462780 T TA); (16374 13462979 A T); (16375 13463178 A
G); (16376 13463233 A G); (16377 13463965 C T); (16378 13463966 A T); (16379 13465167 TA T); (16380
13465269 T A); (16381 13465324 T C); (16382 13465415 G C); (16383 13466193 G A); (16384 13466255
A G); (16385 13466398 C T); (16386 13466698 C T); (16387 13467152 C T); (16388 13467240 T A); (16389
13467503 AT A); (16390 13467813 T C); (16391 13467815 T C); (16392 13467819 T C); (16393 13467821
T C); (16394 13467823 T C); (16395 13467831 T C); (16396 13468145 A T); (16397 13468164 C T); (16398
13468303 T C); (16399 13468405 A T); (16400 13468838 A C); (16401 13468844 C A); (16402 13469208
A G); (16403 13469233 A C); (16404 13469234 C T); (16405 13469275 C T); (16406 13469291 A
C); (16407 13469332 C T); (16408 13469499 T A); (16409 13469799 G C); (16410 13470014 C T); (16411
13470131 A C); (16412 13470164 TA T); (16413 13470307 T C); (16414 13470447 A AT); (16415
13470571 C G); (16416 13471761 T C); (16417 13471784 TAATTG T); (16418 13471789 C T); (16419
13471858 C CT); (16420 13471881 G C); (16421 13471908 C T); (16422 13472060 TTTG T); (16423
13472354 G T); (16424 13472429 C G); (16425 13473183 C T); (16426 13474261 A G); (16427 13475325
T C); (16428 13475411 C A); (16429 13475462 A G); (16430 13475647 C T); (16431 13475727 C
A); (16432 13475865 A G); (16433 13475866 A G); (16434 13476276 T C); (16435 13476570 C A); (16436
13476619 T C); (16437 13476784 G A); (16438 13477144 T C); (16439 13477400 G C); (16440 13477419
A G); (16441 13477988 G A); (16442 13478217 A G); (16443 13478934 C T); (16444 13479072 CG
C); (16445 13479141 C T); (16446 13479557 A G); (16447 13479746 G T); (16448 13480092 C T); (16449
13480142 G A); (16450 13480607 C A); (16451 13480622 G A); (16452 13480682 T C); (16453 13480795
C T); (16454 13480897 A G); (16455 13481634 A AAT); (16456 13481710 A G); (16457 13481961 G
GT); (16458 13482643 T C); (16459 13483414 T G); (16460 13483685 A C); (16461 13483846 A G); (16462
13484597 T C); (16463 13484598 T C); (16464 13484629 G A); (16465 13485367 C T); (16466 13485513
A G); (16467 13485631 C T); (16468 13485728 G A); (16469 13485827 T C); (16470 13486168 G
A); (16471 13486841 A T); (16472 13486871 G A); (16473 13486893 A G); (16474 13487028 A G); (16475
13487237 G A); (16476 13487260 A G); (16477 13487910 T C); (16478 13488059 C T); (16479 13488537
G A); (16480 13488699 C T); (16481 13489001 G A); (16482 13489700 T G); (16483 13490038 A
G); (16484 13490441 G A); (16485 13490698 C T); (16486 13491593 A T); (16487 13492154 A G); (16488
13492516 G A); (16489 13493086 A G); (16490 13493208 T G); (16491 13493240 C T); (16492 13493292
A G); (16493 13493400 C T); (16494 13493887 C T); (16495 13494026 T C); (16496 13494157 C A); (16497
13494324 A G); (16498 13494350 G A); (16499 13494417 G A); (16500 13494444 T C); (16501 13495147
C T); (16502 13495241 T C); (16503 13495417 C CAA); (16504 13495421 C G); (16505 13495921 C
T); (16506 13496123 G A); (16507 13496746 G A); (16508 13496957 T C); (16509 13496979 T A); (16510
13497121 G C); (16511 13497537 G A); (16512 13497666 G A); (16513 13497706 G A); (16514 13498265
C T); (16515 13498359 G C); (16516 13498375 A G); (16517 13498662 C A); (16518 13498772 A
G); (16519 13498821 C T); (16520 13498975 A C); (16521 13499389 A G); (16522 13500811 G A); (16523
13501146 G A); (16524 13502281 C T); (16525 13502549 T C); (16526 13502624 A G); (16527 13502983
T G); (16528 13503168 T C); (16529 13503598 A AG); (16530 13503708 A G); (16531 13504051 AC
A); (16532 13504123 T A); (16533 13504169 A G); (16534 13504953 G T); (16535 13504999 T C); (16536
13505048 A G); (16537 13505083 C T); (16538 13505146 G T); (16539 13505337 A T); (16540 13505722
A G); (16541 13506371 G A); (16542 13506644 T C); (16543 13506661 C G); (16544 13507276 T
TC); (16545 13507424 C T); (16546 13507962 A G); (16547 13508198 G A); (16548 13508213 G A); (16549
13508402 C T); (16550 13508671 T C); (16551 13508903 G A); (16552 13508920 T A); (16553 13509052
A G); (16554 13509173 C T); (16555 13509200 G A); (16556 13509741 A G); (16557 13509758 C
T); (16558 13510549 G A); (16559 13510592 A G); (16560 13510741 A T); (16561 13511035 T G); (16562
13511157 T TA); (16563 13511231 T C); (16564 13511534 T C); (16565 13511727 C A); (16566 13512764
T G); (16567 13512984 GA G); (16568 13513560 A G); (16569 13513679 G GA); (16570 13513743 GA
G); (16571 13514394 A G); (16572 13514595 C T); (16573 13514630 T A); (16574 13515036 G A); (16575
13515048 G T); (16576 13515100 C T); (16577 13515335 C T); (16578 13515469 G A); (16579 13515513
A G); (16580 13515541 C T); (16581 13515567 C T); (16582 13515579 C T); (16583 13516160 G
A); (16584 13516324 C A); (16585 13516326 AT A); (16586 13516485 G A); (16587 13516894 C T); (16588
13516909 C T); (16589 13516985 C T); (16590 13517096 C T); (16591 13518584 G A); (16592 13518614 C
T); (16593 13519023 C CAAACCCT); (16594 13519250 T C); (16595 13519298 T C); (16596 13519438 G
A); (16597 13519760 G C); (16598 13519786 C T); (16599 13519884 G A); (16600 13519950 C T); (16601
13520051 A G); (16602 13520123 G A); (16603 13520477 C T); (16604 13520606 T G); (16605 13520688
C T); (16606 13520714 G GT); (16607 13520842 C T); (16608 13520853 C T); (16609 13521038 C
T); (16610 13521263 A T); (16611 13521328 A G); (16612 13521371 T C); (16613 13521549 A G); (16614
13521560 A C); (16615 13521644 G C); (16616 13521705 A G); (16617 13521713 C T); (16618 13521793
A G); (16619 13521900 C T); (16620 13522040 A C); (16621 13522232 A G); (16622 13522417 T
C); (16623 13522421 T C); (16624 13522865 A G); (16625 13523359 A C); (16626 13523585 A G); (16627
13523745 C G); (16628 13523833 G A); (16629 13523923 A G); (16630 13524129 C T); (16631 13524574
C T); (16632 13524645 G A); (16633 13525105 T C); (16634 13525188 C T); (16635 13525351 T C); (16636
13525688 T C); (16637 13525983 G A); (16638 13526020 T C); (16639 13526054 T C); (16640 13526518
G T); (16641 13526794 C T); (16642 13526826 A C); (16643 13527000 A ATC); (16644 13527110 A
G); (16645 13527119 G A); (16646 13527279 C T); (16647 13527504 T A); (16648 13527620 T G); (16649
13527959 T G); (16650 13529154 A C); (16651 13529156 G GTGTAATGTTAATATTTTTTT); (16652
13529897 C T); (16653 13530454 T C); (16654 13530678 C A); (16655 13530704 G A); (16656 13531327
A G); (16657 13531639 C T); (16658 13531880 A T); (16659 13532101 T C); (16660 13532237 G
A); (16661 13532247 G A); (16662 13532259 A T); (16663 13532451 AC A); (16664 13532528 G
A); (16665 13533567 G T); (16666 13533783 G A); (16667 13534380 A T); (16668 13534381 C T); (16669
13534384 G A); (16670 13535107 T C); (16671 13535149 A G); (16672 13535183 A G); (16673 13535246
C T); (16674 13535567 A G); (16675 13535580 G A); (16676 13535586 C T); (16677 13536501 G
T); (16678 13536519 C T); (16679 13536612 T C); (16680 13536947 G A); (16681 13537318 C T); (16682
13538674 C T); (16683 13538848 C T); (16684 13538949 A G); (16685 13538986 C T); (16686 13539526
A G); (16687 13539674 C T); (16688 13539860 G A); (16689 13540170 T C); (16690 13540450 T
G); (16691 13540989 A G); (16692 13541020 G A); (16693 13541044 G A); (16694 13541887 A G); (16695
13542402 T G); (16696 13542441 T C); (16697 13542559 A C); (16698 13543401 G A); (16699 13543452
G C); (16700 13543457 G C); (16701 13543823 G A); (16702 13543972 C T); (16703 13544046 C
G); (16704 13544239 G A); (16705 13545402 C T); (16706 13546019 C T); (16707 13546052 C T); (16708
13546373 C A); (16709 13546678 T C); (16710 13546695 A G); (16711 13546697 A G); (16712 13546953
A C); (16713 13546957 G T); (16714 13547574 T C); (16715 13549067 T C); (16716 13549179 C T); (16717
13549461 T TC); (16718 13549761 C T); (16719 13549944 C A); (16720 13549951 A T); (16721 13550448
A G); (16722 13550722 A G); (16723 13550774 C A); (16724 13550859 G C); (16725 13551375 T
C); (16726 13551547 T C); (16727 13551616 A C); (16728 13551668 C T); (16729 13552734 C T); (16730
13552814 T C); (16731 13553117 A T); (16732 13554318 T C); (16733 13554546 C T); (16734 13555043 A
C); (16735 13555084 A G); (16736 13555142 T C); (16737 13555168 C T); (16738 13555190 T C); (16739
13555242 CA C); (16740 13555947 A G); (16741 13556110 A C); (16742 13556545 C A); (16743
13556568 C A); (16744 13556640 A G); (16745 13556944 A G); (16746 13557045 G T); (16747 13557200
T A); (16748 13557635 C A); (16749 13557700 T C); (16750 13557728 G T); (16751 13557798 C T); (16752
13557815 T C); (16753 13557919 A G); (16754 13558374 G A); (16755 13558665 G A); (16756 13558847
C T); (16757 13558926 T C); (16758 13559253 T C); (16759 13559385 G T); (16760 13559545 G C); (16761
13559917 T TA); (16762 13560687 A G); (16763 13562100 C T); (16764 13562213 T C); (16765 13562368
T C); (16766 13563021 C T); (16767 13563209 G T); (16768 13563511 C G); (16769 13563741 T C); (16770
13564437 T C); (16771 13564487 G A); (16772 13564590 T C); (16773 13564617 T C); (16774 13566338 T
TGCAA); (16775 13566356 A G); (16776 13566491 A G); (16777 13566707 A G); (16778 13566876 T
C); (16779 13567273 C T); (16780 13567552 C T); (16781 13568294 TC T); (16782 13568386 C T); (16783
13568423 C T); (16784 13568435 T C); (16785 13568482 G C); (16786 13568488 T C); (16787 13568792 A
C); (16788 13569055 T C); (16789 13569083 G A); (16790 13569084 G A); (16791 13569190 G A); (16792
13569195 G C); (16793 13569198 T G); (16794 13569253 T G); (16795 13569261 T C); (16796 13569302
CT); (16797 13570047 C A); (16798 13570150 C T); (16799 13570219 A G); (16800 13570624 C T); (16801
13570727 G C); (16802 13570792 T C); (16803 13570936 C T); (16804 13571089 T TA); (16805 13571110
T G); (16806 13571125 G A); (16807 13571231 T C); (16808 13571275 G A); (16809 13571301 C
G); (16810 13571597 C T); (16811 13571899 G A); (16812 13572083 G T); (16813 13572835 G A); (16814
13572981 A G); (16815 13573436 A T); (16816 13573524 A G); (16817 13574385 A C); (16818 13574421
C T); (16819 13575791 A G); (16820 13575822 G T); (16821 13575906 A G); (16822 13576130 C
T); (16823 13576790 A G); (16824 13576794 C T); (16825 13577849 C T); (16826 13577854 C A); (16827
13578030 G A); (16828 13579953 T C); (16829 13580936 C A); (16830 13581058 G A); (16831 13582046
A G); (16832 13582270 T C); (16833 13582438 A G); (16834 13582574 A G); (16835 13582663 C
A); (16836 13582699 G T); (16837 13582909 C T); (16838 13583319 T C); (16839 13583328 G A); (16840
13583443 G A); (16841 13583720 C A); (16842 13584191 G A); (16843 13584227 C CA); (16844
13584275 G C); (16845 13584354 T C); (16846 13584393 A G); (16847 13584539 G A); (16848 13584571
A C); (16849 13584721 T C); (16850 13584771 T C); (16851 13585262 C T); (16852 13585352 T C); (16853
13585356 A G); (16854 13585549 T G); (16855 13585762 C A); (16856 13586191 A G); (16857 13586599
G A); (16858 13587089 C T); (16859 13587504 A G); (16860 13587795 C T); (16861 13587857 T
C); (16862 13588270 C G); (16863 13596146 T C); (16864 13596195 A C); (16865 13596222 T C); (16866
13596238 C T); (16867 13596315 A G); (16868 13596323 T C); (16869 13596353 GT G); (16870 13596428
G A); (16871 13596429 A C); (16872 13599512 C G); (16873 13599700 A G); (16874 13600196 C
T); (16875 13600348 A G); (16876 13600423 A G); (16877 13601231 T C); (16878 13601279 T G); (16879
13601729 T C); (16880 13601959 C T); (16881 13601991 A G); (16882 13602137 T C); (16883 13602178
G A); (16884 13602316 T C); (16885 13602321 C T); (16886 13602934 T C); (16887 13603446 A
G); (16888 13603510 T C); (16889 13603708 T C); (16890 13603806 T C); (16891 13605607 C A); (16892
13605658 C T); (16893 13605770 C A); (16894 13606002 C T); (16895 13606663 A T); (16896 13606925
G A); (16897 13607576 T A); (16898 13607698 A G); (16899 13607730 T A); (16900 13607946 G
T); (16901 13608090 G A); (16902 13608127 CT C); (16903 13608161 A G); (16904 13608178 G A); (16905
13608231 A G); (16906 13608513 G A); (16907 13608857 C T); (16908 13608922 T C); (16909 13609165
G A); (16910 13609167 G A); (16911 13610073 A G); (16912 13613750 G T); (16913 13613929 A
C); (16914 13618997 T A); (16915 13619124 G A); (16916 13619229 T C); (16917 13619429 T G); (16918
13619438 T C); (16919 13619461 A G); (16920 13619754 G A); (16921 13619813 T G); (16922 13619850
T C); (16923 13619936 A G); (16924 13620003 G A); (16925 13620039 A G); (16926 13620061 T
C); (16927 13621055 A T); (16928 13621198 T C); (16929 13621271 T C); (16930 13621453 C
CTG); (16931 13621818 C T); (16932 13621963 AT A); (16933 13622250 A G); (16934 13622328 T
C); (16935 13622672 G A); (16936 13623537 T C); (16937 13623699 T C); (16938 13623880 C T); (16939
13623992 C T); (16940 13624011 G T); (16941 13624075 T C); (16942 13624177 C T); (16943 13624215 T
C); (16944 13624531 T C); (16945 13625056 T C); (16946 13625165 G A); (16947 13625507 G A); (16948
13625841 A G); (16949 13625853 A C); (16950 13625930 A G); (16951 13626085 G A); (16952 13626175
T C); (16953 13626217 C G); (16954 13626817 G A); (16955 13627167 A C); (16956 13628197 T
C); (16957 13628206 C T); (16958 13628460 T A); (16959 13628585 A T); (16960 13628812 G A); (16961
13629095 G A); (16962 13629867 G A); (16963 13630047 C T); (16964 13630088 C T); (16965 13630431
A T); (16966 13630851 G T); (16967 13631141 T A); (16968 13631439 C T); (16969 13631450 C A); (16970
13631494 T C); (16971 13631517 C G); (16972 13632262 C T); (16973 13632307 G A); (16974 13632494
A G); (16975 13632938 T C); (16976 13633307 A C); (16977 13633321 G A); (16978 13633525 C
G); (16979 13633530 AT A); (16980 13633531 C A); (16981 13634739 T C); (16982 13634791 T
TC); (16983 13634946 G A); (16984 13635247 C T); (16985 13635294 T C); (16986 13635440 T C); (16987
13635624 G A); (16988 13635713 C T); (16989 13635857 TA T); (16990 13635861 T G); (16991 13635947
T C); (16992 13636078 T A); (16993 13636169 G A); (16994 13636778 A T); (16995 13636852 C T); (16996
13636862 T TATATATATATATATACAC); (16997 13636865 A T); (16998 13636913 A T); (16999 13637194
C T); (17000 13637317 T TTA); (17001 13637328 G A); (17002 13637644 T TA); (17003 13637867 AC
A); (17004 13638125 A AT); (17005 13638733 C T); (17006 13638737 T A); (17007 13638838 A
AGAAAAAAAGT); (17008 13639591 G A); (17009 13639768 A C); (17010 13639832 C T); (17011
13640322 A T); (17012 13640670 T G); (17013 13640877 G A); (17014 13642255 G A); (17015 13642293
T C); (17016 13643074 A G); (17017 13643326 C T); (17018 13644253 TA T); (17019 13644380 A
C); (17020 13644416 G C); (17021 13644688 T A); (17022 13644854 C T); (17023 13644937 T TA); (17024
13645121 T G); (17025 13645616 C T); (17026 13645702 T G); (17027 13645767 C A); (17028 13646922
A G); (17029 13647366 T C); (17030 13647386 C A); (17031 13647560 C T); (17032 13647582 C T); (17033
13647791 G A); (17034 13647810 C T); (17035 13650693 G A); (17036 13651049 T C); (17037 13651086
G A); (17038 13651365 A C); (17039 13651371 G A); (17040 13651406 T C); (17041 13652291 G
A); (17042 13652319 T G); (17043 13652568 C T); (17044 13652945 G A); (17045 13653301 C A); (17046
13653504 A C); (17047 13654063 T C); (17048 13654087 T C); (17049 13654562 T G); (17050 13654579
G A); (17051 13655287 T C); (17052 13655323 G A); (17053 13655422 A G); (17054 13655535 G
C); (17055 13655693 A G); (17056 13655706 AT A); (17057 13655917 A G); (17058 13656607 A
G); (17059 13656727 T C); (17060 13656941 C T); (17061 13657975 G A); (17062 13658116 C T); (17063
13658271 G C); (17064 13658345 T C); (17065 13658363 C A); (17066 13658496 T C); (17067 13658755
A G); (17068 13658793 C T); (17069 13658941 C T); (17070 13658981 C T); (17071 13658992 G
A); (17072 13659564 T C); (17073 13659569 C T); (17074 13659846 C T); (17075 13660198 T C); (17076
13660199 A G); (17077 13660285 G A); (17078 13660452 C G); (17079 13660603 C T); (17080 13661166
T C); (17081 13661293 G A); (17082 13661465 T C); (17083 13661562 T C); (17084 13661849 G
A); (17085 13662123 G A); (17086 13662254 C T); (17087 13662473 C T); (17088 13662507 A G); (17089
13662626 A G); (17090 13662768 T C); (17091 13662828 G A); (17092 13663386 C T); (17093 13663389
A G); (17094 13663843 A G); (17095 13664959 C T); (17096 13665140 T C); (17097 13665264 A
G); (17098 13665431 C T); (17099 13665446 C A); (17100 13665575 G A); (17101 13666109 T A); (17102
13667337 T G); (17103 13667347 A G); (17104 13667571 A AT); (17105 13667963 G A); (17106
13668694 A G); (17107 13670499 T G); (17108 13673059 T C); (17109 13673645 G A); (17110 13673676
C A); (17111 13674785 C A); (17112 13674903 A G); (17113 13675201 G A); (17114 13675237 A
G); (17115 13675848 C T); (17116 13676043 A G); (17117 13676244 G A); (17118 13676791 T C); (17119
13677135 C T); (17120 13677551 G A); (17121 13677561 C T); (17122 13677594 C T); (17123 13677743
G A); (17124 13677900 T C); (17125 13677969 G A); (17126 13678368 T C); (17127 13678491 G
A); (17128 13678538 C T); (17129 13678554 G A); (17130 13679199 A G); (17131 13679263 A G); (17132
13679515 T C); (17133 13679582 G A); (17134 13679612 A G); (17135 13679763 G T); (17136 13679870
G A); (17137 13679872 T C); (17138 13679932 G A); (17139 13680045 A G); (17140 13680057 G
A); (17141 13680092 C T); (17142 13680149 C G); (17143 13680305 T A); (17144 13680393 A G); (17145
13680664 T TA); (17146 13680710 G T); (17147 13680769 A G); (17148 13680797 G A); (17149
13680810 T C); (17150 13680924 A AT); (17151 13681090 T C); (17152 13681529 C T); (17153 13681544
A G); (17154 13682097 G A); (17155 13682622 T C); (17156 13682639 A G); (17157 13682708 C
T); (17158 13682839 C T); (17159 13683192 C T); (17160 13683318 G T); (17161 13683406 T C); (17162
13683931 C A); (17163 13684366 T C); (17164 13684437 G A); (17165 13684552 T C); (17166 13684568
C T); (17167 13684699 T C); (17168 13684760 C T); (17169 13684794 T C); (17170 13684945 G A); (17171
13685059 A G); (17172 13685202 T C); (17173 13685237 A G); (17174 13685274 G C); (17175 13685474
G A); (17176 13685540 A G); (17177 13685559 C T); (17178 13685617 G A); (17179 13685631 A
G); (17180 13685992 G A); (17181 13686070 T G); (17182 13686081 T C); (17183 13686103 T C); (17184
13686187 A G); (17185 13686235 C A); (17186 13686279 T C); (17187 13686617 T C); (17188 13686655
G A); (17189 13686676 A T); (17190 13686860 T A); (17191 13686911 T A); (17192 13687151 T C); (17193
13688956 T C); (17194 13689192 C A); (17195 13689270 T C); (17196 13689693 C T); (17197 13689797 A
G); (17198 13689872 C CT); (17199 13690044 G A); (17200 13690291 G A); (17201 13690696 A
C); (17202 13690713 G C); (17203 13690764 T C); (17204 13690877 G A); (17205 13691140 A T); (17206
13691842 G C); (17207 13691993 G C); (17208 13692276 A T); (17209 13692392 C T); (17210 13692425
C A); (17211 13693163 G A); (17212 13694054 C T); (17213 13694103 A G); (17214 13694496 T
A); (17215 13694557 C G); (17216 13694584 C G); (17217 13694603 G A); (17218 13694679 T C); (17219
13695031 G A); (17220 13695219 T G); (17221 13695402 A G); (17222 13695459 C T); (17223 13695535
C G); (17224 13696255 T C); (17225 13696984 T G); (17226 13696988 C T); (17227 13697126 C T); (17228
13697195 G A); (17229 13697201 T C); (17230 13697431 T C); (17231 13697545 C T); (17232 13698019 T
C); (17233 13698184 A G); (17234 13698305 C G); (17235 13698361 G T); (17236 13698580 A G); (17237
13698792 A G); (17238 13698872 A G); (17239 13699157 G A); (17240 13699332 T G); (17241 13699419
G T); (17242 13700468 A C); (17243 13700531 G A); (17244 13700645 G A); (17245 13700828 A
C); (17246 13700884 G A); (17247 13701149 C A); (17248 13701189 C T); (17249 13702280 A
AAAT); (17250 13702307 A C); (17251 13702883 A G); (17252 13703452 G T); (17253 13703481 A
G); (17254 13703632 T C); (17255 13704046 A G); (17256 13704305 T C); (17257 13704719 A G); (17258
13704756 A G); (17259 13705003 C T); (17260 13705074 G A); (17261 13705743 A T); (17262 13706184
G A); (17263 13706186 T C); (17264 13706356 G A); (17265 13706448 G A); (17266 13706663 A
G); (17267 13706931 T C); (17268 13706986 C A); (17269 13707024 C T); (17270 13707057 A T); (17271
13707097 A G); (17272 13707398 T A); (17273 13707556 A G); (17274 13707884 C G); (17275 13708000
A G); (17276 13708759 C A); (17277 13708954 C T); (17278 13709601 A G); (17279 13711830 T
C); (17280 13711907 A G); (17281 13712624 T G); (17282 13712629 C CA); (17283 13712803
ACCCCCCCCCCC A); (17284 13712805 A G); (17285 13712806 G T); (17286 13714288 T C); (17287
13715971 C A); (17288 13716673 T C); (17289 13716704 T G); (17290 13729274 G T); (17291 13729872
G A); (17292 13729931 A ATT); (17293 13730782 G T); (17294 13731712 A T); (17295 13731863 C
T); (17296 13732834 T C); (17297 13733256 G A); (17298 13733455 T A); (17299 13734217 C T); (17300
13734280 T C); (17301 13734571 T C); (17302 13734617 G A); (17303 13734669 G A); (17304 13734791
A G); (17305 13734837 C A); (17306 13735360 A G); (17307 13735409 T C); (17308 13735432 G
A); (17309 13735708 T C); (17310 13736052 T C); (17311 13736093 T C); (17312 13736864 A C); (17313
13737361 A G); (17314 13737733 A G); (17315 13738076 C T); (17316 13738663 A G); (17317 13739616
T C); (17318 13739781 T C); (17319 13739786 C T); (17320 13740436 TC T); (17321 13740549 G
T); (17322 13740821 T C); (17323 13740875 C T); (17324 13740941 C T); (17325 13741131 C T); (17326
13741415 T C); (17327 13741641 G A); (17328 13741737 A G); (17329 13741804 G A); (17330 13741822
T C); (17331 13741891 T C); (17332 13741923 G A); (17333 13741943 A T); (17334 13742131 C T); (17335
13742298 T C); (17336 13742375 A G); (17337 13742597 T G); (17338 13742640 G A); (17339 13742744
G A); (17340 13743029 A G); (17341 13743083 C T); (17342 13743579 C T); (17343 13743619 G
A); (17344 13743703 T C); (17345 13743761 T C); (17346 13743827 G A); (17347 13744170 G T); (17348
13744200 A G); (17349 13744232 G A); (17350 13744355 A G); (17351 13744410 A G); (17352 13744744
C T); (17353 13744872 A G); (17354 13745111 A G); (17355 13745197 T C); (17356 13745289 G
A); (17357 13745379 C T); (17358 13745387 G A); (17359 13745988 T G); (17360 13746153 T C); (17361
13746272 A G); (17362 13746408 A G); (17363 13746481 G T); (17364 13746688 T C); (17365 13746821
T C); (17366 13747249 T C); (17367 13747549 G A); (17368 13747560 G T); (17369 13748092 A
G); (17370 13748278 T C); (17371 13748290 T C); (17372 13748485 A C); (17373 13748683 C G); (17374
13748761 A T); (17375 13748912 T C); (17376 13749173 G A); (17377 13749404 A G); (17378 13749467
A C); (17379 13749523 G A); (17380 13749524 T C); (17381 13749608 C T); (17382 13749636 T C); (17383
13749857 G A); (17384 13749917 T C); (17385 13750100 C T); (17386 13750498 A C); (17387 13750618
G A); (17388 13750931 G A); (17389 13751529 T C); (17390 13752062 A G); (17391 13752177 G
A); (17392 13752316 T C); (17393 13752745 G A); (17394 13753331 A G); (17395 13753337 A G); (17396
13753457 G A); (17397 13754568 C T); (17398 13754853 A G); (17399 13754854 T C); (17400 13754904
A G); (17401 13755249 C T); (17402 13755280 A G); (17403 13755330 G A); (17404 13755656 G
A); (17405 13755778 C T); (17406 13755869 A G); (17407 13755977 G A); (17408 13756741 T C); (17409
13756783 T C); (17410 13757096 T C); (17411 13757141 T TCCGCTTCTCCA); (17412 13757177 T
C); (17413 13757209 A G); (17414 13757219 C T); (17415 13757279 G GGCCC); (17416 13760666 T
G); (17417 13761384 A T); (17418 13761974 ATT A); (17419 13762000 A AAATAGGGGG); (17420
13762071 G A); (17421 13762901 A AATGAATT); (17422 13764153 G A); (17423 13765131 A T); (17424
13766131 C T); (17425 13766950 A G); (17426 13766991 T C); (17427 13768550 G C); (17428 13769131
A G); (17429 13769243 G C); (17430 13769792 A C); (17431 13771083 G A); (17432 13771161 CT
C); (17433 13771329 C A); (17434 13772200 C T); (17435 13772281 A G); (17436 13773585 T C); (17437
13775277 T C); (17438 13775942 G A); (17439 13776045 C T); (17440 13776256 G A); (17441 13776269
C A); (17442 13776277 C A); (17443 13777121 G C); (17444 13777555 T C); (17445 13777977 A
G); (17446 13778494 A G); (17447 13779084 C T); (17448 13779234 A G); (17449 13780025 G A); (17450
13784959 G A); (17451 13792392 A G); (17452 13793553 C G); (17453 13793555 T C); (17454 13793706
C T); (17455 13794108 C T); (17456 13794120 C T); (17457 13794313 C T); (17458 13795228 A G); (17459
13795259 G A); (17460 13795641 A G); (17461 13795765 C T); (17462 13796074 G A); (17463 13796084
A G); (17464 13796200 C T); (17465 13796302 C T); (17466 13796625 C A); (17467 13796769 T C); (17468
13796991 A G); (17469 13797045 C T); (17470 13797245 T A); (17471 13797304 C T); (17472 13797347
C T); (17473 13797398 T C); (17474 13797911 A G); (17475 13798541 T G); (17476 13798717 C T); (17477
13798728 G A); (17478 13799149 CCTGGCCCT C); (17479 13799319 T C); (17480 13799331 C T); (17481
13799864 C G); (17482 13800181 T G); (17483 13800238 A T); (17484 13800699 A T); (17485 13801519
A G); (17486 13802181 A G); (17487 13803421 T C); (17488 13805910 G A); (17489 13806156 C
T); (17490 13807277 C T); (17491 13807895 A G); (17492 13808024 A G); (17493 13808059 G C); (17494
13808647 T A); (17495 13809012 C T); (17496 13810279 A G); (17497 13810777 T G); (17498 13810901
C T); (17499 13810918 G A); (17500 13810993 G A); (17501 13811342 T C); (17502 13811456 G
A); (17503 13812305 A C); (17504 13812936 T C); (17505 13813648 C A); (17506 13813711 C T); (17507
13813917 A G); (17508 13814457 G A); (17509 13814881 A G); (17510 13815538 G A); (17511 13817224
T C); (17512 13817599 T C); (17513 13818138 C T); (17514 13818328 C T); (17515 13818928 T C); (17516
13818931 T C); (17517 13819608 TA T); (17518 13819826 G A); (17519 13820528 G A); (17520 13820530
T C); (17521 13820823 A G); (17522 13821307 C T); (17523 13821519 G A); (17524 13822123 G
A); (17525 13822380 A C); (17526 13825200 T C); (17527 13825317 C T); (17528 13825680 C T); (17529
13826896 C T); (17530 13827248 C T); (17531 13827602 C T); (17532 13827641 G T); (17533 13828028 A
C); (17534 13828116 T C); (17535 13829251 C T); (17536 13829419 A T); (17537 13829587 C T); (17538
13829660 A G); (17539 13830011 T G); (17540 13830533 G C); (17541 13831155 G C); (17542 13831164
G A); (17543 13831216 T G); (17544 13831297 A G); (17545 13831633 C T); (17546 13831684 A
G); (17547 13831880 A G); (17548 13831960 G C); (17549 13832067 G A); (17550 13832219 A G); (17551
13832323 T C); (17552 13832737 A G); (17553 13832814 G A); (17554 13832824 C T); (17555 13832880
T C); (17556 13832975 G A); (17557 13833149 G T); (17558 13833533 A G); (17559 13833563 T
C); (17560 13833950 T C); (17561 13834038 G C); (17562 13835004 A G); (17563 13835363 T C); (17564
13836169 CA C); (17565 13836393 T C); (17566 13836617 G C); (17567 13837206 T
TATAATAATAATAATAATAATAATA); (17568 13839056 T TAC); (17569 13839560 T G); (17570 13839903 A
G); (17571 13840836 T A); (17572 13842118 A G); (17573 13842314 G A); (17574 13843394 C T); (17575
13844198 A G); (17576 13844557 T G); (17577 13844811 G A); (17578 13844959 TG T); (17579
13845058 C G); (17580 13845079 G A); (17581 13845155 C T); (17582 13845210 G A); (17583 13845214
C T); (17584 13845238 A G); (17585 13845260 A G); (17586 13846405 G T); (17587 13846882 T
TA); (17588 13847997 A G); (17589 13849485 T C); (17590 13849805 AT A); (17591 13849961 C
G); (17592 13850319 A G); (17593 13850349 C T); (17594 13853644 C T); (17595 13856558 A G); (17596
13856766 C T); (17597 13856793 A G); (17598 13857672 C T); (17599 13857822 G A); (17600 13859134
C T); (17601 13860121 A G); (17602 13860195 T C); (17603 13860259 G A); (17604 13860937 A
G); (17605 13861441 TG T); (17606 13861940 G T); (17607 13861951 G C); (17608 13862427 G
A); (17609 13862429 G A); (17610 13863177 C A); (17611 13863377 G T); (17612 13863546 C A); (17613
13863882 C T); (17614 13863911 T C); (17615 13864524 T G); (17616 13865020 T C); (17617 13866255 C
T); (17618 13866515 A G); (17619 13866586 T C); (17620 13866618 G C); (17621 13866736 A C); (17622
13866824 C T); (17623 13869836 C A); (17624 13870061 C T); (17625 13870180 C T); (17626 13870211 G
A); (17627 13870233 G C); (17628 13871984 T C); (17629 13872589 A G); (17630 13873099 G A); (17631
13873163 T G); (17632 13873218 G C); (17633 13873290 C T); (17634 13873315 T A); (17635 13873340
A C); (17636 13873823 C T); (17637 13875446 AG A); (17638 13877105 T C); (17639 13877212 G
T); (17640 13878030 C T); (17641 13878361 A G); (17642 13879381 C T); (17643 13879595 G A); (17644
13879649 A C); (17645 13879702 C T); (17646 13880183 C T); (17647 13881419 C A); (17648 13882164 C
A); (17649 13882690 A T); (17650 13882811 A G); (17651 13883088 T C); (17652 13883159 G T); (17653
13883274 G A); (17654 13887179 C T); (17655 13889275 G A); (17656 13889420 C T); (17657 13890414
C A); (17658 13890446 T G); (17659 13891884 C T); (17660 13892451 T TAAATAA); (17661 13892554 T
A); (17662 13892572 T C); (17663 13892611 A G); (17664 13892641 G T); (17665 13894021 G T); (17666
13897547 T A); (17667 13897818 T A); (17668 13898548 C T); (17669 13899419 C T); (17670 13899488
AATACCACAAATTTGAT A); (17671 13900943 T C); (17672 13901062 G A); (17673 13901439 G A); (17674
13901474 G C); (17675 13901879 A G); (17676 13901977 A G); (17677 13902159 G A); (17678 13902378
G A); (17679 13902512 C T); (17680 13903073 T C); (17681 13903466 G A); (17682 13904579 G
A); (17683 13905616 G GT); (17684 13906025 A G); (17685 13906176 C T); (17686 13906489 G
A); (17687 13907632 A G); (17688 13907708 A G); (17689 13908339 T G); (17690 13909481 T C); (17691
13910058 C A); (17692 13910963 A G); (17693 13911434 G A); (17694 13911631 A T); (17695 13912298
T G); (17696 13912340 T C); (17697 13912448 C T); (17698 13912502 A G); (17699 13913063 G
A); (17700 13913538 C T); (17701 13914647 T G); (17702 13914708 TTTGA T); (17703 13914917 G
A); (17704 13916568 G A); (17705 13916797 G A); (17706 13916839 C T); (17707 13918930 G A); (17708
13918975 G A); (17709 13919347 T C); (17710 13919558 T G); (17711 13919649 G T); (17712 13920669
T C); (17713 13921686 T C); (17714 13922461 T G); (17715 13923516 G T); (17716 13924424 C T); (17717
13924684 C G); (17718 13924730 T G); (17719 13924796 T C); (17720 13925597 A G); (17721 13925869
C A); (17722 13926041 G A); (17723 13929822 A T); (17724 13930100 A T); (17725 13930112 G
A); (17726 13930571 A G); (17727 13931467 T C); (17728 13932399 C T); (17729 13932408 T C); (17730
13932435 A G); (17731 13932736 G A); (17732 13933573 G A); (17733 13934428 C T); (17734 13934928
G A); (17735 13936596 A G); (17736 13936736 T C); (17737 13936787 C T); (17738 13937209 T
G); (17739 13937460 GCAAAATC G); (17740 13939252 G T); (17741 13939536 G A); (17742 13942320 A
G); (17743 13942669 G A); (17744 13943260 G A); (17745 13944393 T C); (17746 13945351 G A); (17747
13946921 A G); (17748 13947290 A G); (17749 13947818 G A); (17750 13948016 G A); (17751 13948065
C G); (17752 13948434 G C); (17753 13948701 AAG A); (17754 13949384 G C); (17755 13949439 G
A); (17756 13950524 C T); (17757 13952241 C G); (17758 13952881 C G); (17759 13952898 A G); (17760
13953643 C T); (17761 13954746 T C); (17762 13956776 C T); (17763 13957258 A G); (17764 13958611
A T); (17765 13959207 T C); (17766 13959606 T A); (17767 13960391 A T); (17768 13960472 C A); (17769
13963433 G A); (17770 13963485 T C); (17771 13963896 A T); (17772 13964058 A G); (17773 13964147
T G); (17774 13964527 A G); (17775 13965510 G A); (17776 13965886 C T); (17777 13967695 T
A); (17778 13967863 T G); (17779 13967888 A G); (17780 13967996 A C); (17781 13968611 T C); (17782
13969324 G A); (17783 13969564 A T); (17784 13971016 C T); (17785 13971874 A G); (17786 13973617
T G); (17787 13973656 G A); (17788 13974321 C A); (17789 13974577 C A); (17790 13974630 G
A); (17791 13974720 AAAATG A); (17792 13974956 C G); (17793 13976384 T A); (17794 13976819 A
C); (17795 13976953 T C); (17796 13977376 T A); (17797 13977490 A AAC); (17798 13978025 ATG
A); (17799 13981571 A G); (17800 13981747 G A); (17801 13982424 A G); (17802 13982510 A
AC); (17803 13982945 G T); (17804 13983032 A G); (17805 13983040 A C); (17806 13983338 T C); (17807
13983354 A G); (17808 13983654 C T); (17809 13983793 T C); (17810 13983955 G A); (17811 13984370
C T); (17812 13984497 T C); (17813 13984680 C G); (17814 13984719 T C); (17815 13984825 C T); (17816
13985431 CA C); (17817 13985678 T G); (17818 13985960 T G); (17819 13986121 C T); (17820 13986166
T A); (17821 13986645 C T); (17822 13987540 T A); (17823 13987966 C T); (17824 13987984 A T); (17825
13988002 G A); (17826 13988158 C T); (17827 13988185 T C); (17828 13988287 T C); (17829 13988311
GC G); (17830 13989195 T C); (17831 13990047 T C); (17832 13990100 C T); (17833 13990103 T
C); (17834 13990144 T C); (17835 13990341 A G); (17836 13990619 T C); (17837 13990685 T A); (17838
13990778 T C); (17839 13990966 C T); (17840 13991193 T G); (17841 13991744 C T); (17842 13992776
G T); (17843 13996004 A G); (17844 13996018 A G); (17845 13997671 G A); (17846 13997704 A
G); (17847 13997707 A T); (17848 13997724 G T); (17849 13997737 A G); (17850 13997790 C T); (17851
13998160 A G); (17852 13999628 G T); (17853 13999662 G C); (17854 13999676 G A); (17855 13999720
G A); (17856 13999812 G A); (17857 14000726 C T); (17858 14000758 T C); (17859 14000832 A
T); (17860 14000930 T C); (17861 14000947 G A); (17862 14000950 C T); (17863 14003199 G A); (17864
14003495 C G); (17865 14003552 T C); (17866 14003703 A G); (17867 14003896 T C); (17868 14003931
A T); (17869 14004112 G A); (17870 14004221 G A); (17871 14004497 C T); (17872 14005032 G
T); (17873 14005054 T C); (17874 14005138 G A); (17875 14005165 C T); (17876 14005269 T G); (17877
14005312 A G); (17878 14005716 T C); (17879 14005719 A G); (17880 14006296 T C); (17881 14006593
C G); (17882 14006785 A G); (17883 14007319 A G); (17884 14008898 C T); (17885 14009295 A
T); (17886 14010610 A T); (17887 14010768 T C); (17888 14010779 A T); (17889 14011009 T A); (17890
14011625 G A); (17891 14014264 T C); (17892 14015463 T TGAG); (17893 14016705 C G); (17894
14016733 C A); (17895 14016746 C CTT); (17896 14018426 G A); (17897 14018468 A G); (17898
14020416 A ATC); (17899 14021036 G T); (17900 14022468 T G); (17901 14023742 C A); (17902
14025357 ATT A); (17903 14025822 A AT); (17904 14026295 T C); (17905 14026960 A ATT); (17906
14027268 G C); (17907 14027303 C T); (17908 14027334 C T); (17909 14027335 A G); (17910 14027351
C T); (17911 14027394 C T); (17912 14027411 G A); (17913 14027512 A G); (17914 14027737 G
A); (17915 14027921 A G); (17916 14028062 T G); (17917 14028157 C T); (17918 14028393 A G); (17919
14028611 G T); (17920 14029751 G A); (17921 14029865 G A); (17922 14030752 GA G); (17923
14030817 T G); (17924 14031015 T A); (17925 14031254 T C); (17926 14031315 C T); (17927 14031462
G T); (17928 14031476 C T); (17929 14032689 C A); (17930 14033886 A G); (17931 14034267 C
G); (17932 14034293 T G); (17933 14034350 G C); (17934 14034980 T C); (17935 14035026 T C); (17936
14035179 C T); (17937 14035529 A G); (17938 14035822 C T); (17939 14036005 C G); (17940 14036149
A G); (17941 14036562 G GA); (17942 14036630 G A); (17943 14036727 G A); (17944 14036924 GA
G); (17945 14037204 C A); (17946 14037311 T G); (17947 14037395 A G); (17948 14039163 A T); (17949
14039170 C A); (17950 14039319 T G); (17951 14040289 T C); (17952 14040437 A G); (17953 14041607
G C); (17954 14043786 GA G); (17955 14044849 CAGTG C); (17956 14044938 A G); (17957 14045701 A
G); (17958 14046567 G A); (17959 14046779 ATTTG A); (17960 14047127 T G); (17961 14048102 T
TAG); (17962 14048105 A G); (17963 14048614 C T); (17964 14048679 A G); (17965 14048742 T
C); (17966 14048827 A C); (17967 14049021 A T); (17968 14049068 C T); (17969 14049167 G A); (17970
14049214 C T); (17971 14049920 G C); (17972 14050440 T C); (17973 14053603 C T); (17974 14053670 T
G); (17975 14054140 T C); (17976 14054309 A G); (17977 14060847 C T); (17978 14061513 T C); (17979
14062232 T C); (17980 14062267 A G); (17981 14062353 A C); (17982 14062462 C T); (17983 14062508
G A); (17984 14062639 C T); (17985 14062644 A G); (17986 14062775 G T); (17987 14063280 T
A); (17988 14063283 T A); (17989 14063286 T A); (17990 14063289 A AATAATAATTATT); (17991
14063644 C T); (17992 14064051 A G); (17993 14064314 G A); (17994 14064354 C T); (17995 14064396
G A); (17996 14064413 G A); (17997 14064600 T C); (17998 14064791 G T); (17999 14064975 C
A); (18000 14065116 TAATTTTTTTGTCATTTCTCA T); (18001 14065285 AT A); (18002 14065705 G
T); (18003 14066764 G A); (18004 14067413 T TA); (18005 14067515 A G); (18006 14067929 C T); (18007
14068020 G T); (18008 14068294 G C); (18009 14068411 T G); (18010 14069476 A G); (18011 14070241
G T); (18012 14070490 G A); (18013 14073229 C T); (18014 14073866 A G); (18015 14074150 C
T); (18016 14074531 C T); (18017 14075598 G A); (18018 14076609 A C); (18019 14078169 T
TTGAC); (18020 14078280 C T); (18021 14079228 AC A); (18022 14079311 T G); (18023 14079451 G
GA); (18024 14079598 A C); (18025 14079636 T A); (18026 14079864 C G); (18027 14079891 C T); (18028
14080002 T C); (18029 14080032 T A); (18030 14080751 A AT); (18031 14080989 G A); (18032 14084026
T C); (18033 14084681 T G); (18034 14084794 A G); (18035 14085876 T C); (18036 14086196 C T); (18037
14087115 C T); (18038 14087155 T C); (18039 14087587 C T); (18040 14088955 T C); (18041 14088965 C
T); (18042 14089074 C T); (18043 14089359 C T); (18044 14089462 G A); (18045 14089925 T G); (18046
14090396 T C); (18047 14091240 A G); (18048 14091768 T C); (18049 14092863 C T); (18050 14093013 C
G); (18051 14093266 A G); (18052 14093767 T A); (18053 14094657 T C); (18054 14094689 G A); (18055
14095220 A G); (18056 14095490 A G); (18057 14096390 G A); (18058 14097490 G A); (18059 14097790
G A); (18060 14098540 G A); (18061 14099919 A G); (18062 14100163 T C); (18063 14100383 C
T); (18064 14102191 T C); (18065 14102682 G A); (18066 14102953 G A); (18067 14104214 A G); (18068
14104239 C T); (18069 14104273 G A); (18070 14104317 A G); (18071 14104482 C T); (18072 14104661
C T); (18073 14105027 C T); (18074 14105982 G A); (18075 14107584 C CT); (18076 14109886 T
TA); (18077 14110824 A G); (18078 14112590 A G); (18079 14112635 A G); (18080 14114920 G
A); (18081 14115798 C T); (18082 14116036 C T); (18083 14116330 C T); (18084 14116351 GT G); (18085
14116465 ATTACTAGATAACTCATTTATTG A); (18086 14116582 G A); (18087 14116639 AT A); (18088
14117107 T C); (18089 14117134 C T); (18090 14117597 C G); (18091 14118470 T C); (18092 14118540
TCAAACATGTC T); (18093 14118544 A G); (18094 14118637 C G); (18095 14118768 G A); (18096
14118836 C T); (18097 14119498 G A); (18098 14119767 C T); (18099 14119822 C T); (18100 14119828
A G); (18101 14119857 G C); (18102 14120450 A G); (18103 14121751 AATAT A); (18104 14122106 A
G); (18105 14123849 G C); (18106 14124104 C T); (18107 14124767 G A); (18108 14125009 T C); (18109
14125268 C T); (18110 14125855 A G); (18111 14128536 T G); (18112 14128596 T G); (18113 14129343
G A); (18114 14130233 A G); (18115 14130771 G A); (18116 14131988 G C); (18117 14132039 C
A); (18118 14132200 C T); (18119 14132407 A T); (18120 14132784 C G); (18121 14132834 C A); (18122
14132986 G A); (18123 14133086 G T); (18124 14134286 C T); (18125 14134416 A G); (18126 14134641
A G); (18127 14134814 C T); (18128 14134898 G A); (18129 14135088 C T); (18130 14135452 T
A); (18131 14135499 T C); (18132 14135971 T A); (18133 14136020 AT A); (18134 14137691 G T); (18135
14137754 T A); (18136 14138132 A G); (18137 14138447 T C); (18138 14138478 T C); (18139 14138704 T
C); (18140 14138868 T C); (18141 14144176 T TA); (18142 14144565 A T); (18143 14146191 A C); (18144
14148052 A G); (18145 14148627 G A); (18146 14148719 A G); (18147 14149173 TAAAAAGCCC
T); (18148 14150260 A G); (18149 14150267 G A); (18150 14151214 G GCAC); (18151 14152332 T
TA); (18152 14152730 G A); (18153 14152864 T C); (18154 14153130 C T); (18155 14153418 T G); (18156
14154380 T A); (18157 14154842 C T); (18158 14155025 A G); (18159 14155114 A G); (18160 14155569
TTG); (18161 14155724 A T); (18162 14155727 A G); (18163 14155728 G C); (18164 14156046 A
G); (18165 14157408 T G); (18166 14160747 A G); (18167 14160748 T C); (18168 14160892 G A); (18169
14163091 C A); (18170 14163302 G T); (18171 14163721 A G); (18172 14163865 C A); (18173 14164293
C T); (18174 14164379 A C); (18175 14164910 G A); (18176 14165045 G A); (18177 14165060 G
A); (18178 14165063 C T); (18179 14165088 T G); (18180 14165109 T C); (18181 14165833 G T); (18182
14166822 G T); (18183 14168002 A G); (18184 14168423 C T); (18185 14170101 C A); (18186 14170246
G GA); (18187 14170526 C CCTGAATCCCCGCGCG); (18188 14170566 C T); (18189 14172239 C T); (18190
14174406 G A); (18191 14176391 C T); (18192 14176729 A G); (18193 14177093 C T); (18194 14177306
G A); (18195 14178364 T TGA); (18196 14178486 A G); (18197 14178490 G A); (18198 14179164 A
G); (18199 14179397 T C); (18200 14179400 A G); (18201 14179806 G T); (18202 14180427 T C); (18203
14180573 T C); (18204 14180853 A G); (18205 14181242 T C); (18206 14181435 T C); (18207 14181596
A C); (18208 14181772 T C); (18209 14181814 T C); (18210 14182137 C T); (18211 14182262 T C); (18212
14182862 C T); (18213 14182893 G A); (18214 14183173 T C); (18215 14183673 G T); (18216 14183836
G A); (18217 14186299 T G); (18218 14186888 A G); (18219 14187030 A G); (18220 14187189 T
C); (18221 14188170 G C); (18222 14188667 T C); (18223 14189271 T C); (18224 14190329 A G); (18225
14191465 T C); (18226 14191743 G A); (18227 14191801 GA G); (18228 14192351 A G); (18229
14192669 T C); (18230 14193026 C T); (18231 14193626 T C); (18232 14194458 T C); (18233 14194611 A
G); (18234 14195289 G A); (18235 14195306 C T); (18236 14195307 A C); (18237 14195371 G A); (18238
14195596 A G); (18239 14196075 C CT); (18240 14196386 G A); (18241 14196410 G T); (18242
14196681 T G); (18243 14196790 A G); (18244 14197084 G A); (18245 14198402 A G); (18246 14198556
A G); (18247 14199117 G T); (18248 14199238 C A); (18249 14199272 C G); (18250 14199578 T
TA); (18251 14199886 A G); (18252 14200033 A G); (18253 14200134 A G); (18254 14201168 T
A); (18255 14201904 G GTGAACAATGTAATCGATTACT); (18256 14203273 G A); (18257 14203274 A
T); (18258 14203782 A G); (18259 14204019 C T); (18260 14204032 A T); (18261 14204295 G A); (18262
14204641 G A); (18263 14204685 T C); (18264 14204695 G A); (18265 14204709 A G); (18266 14205081
C T); (18267 14205187 G A); (18268 14205216 A C); (18269 14205232 T C); (18270 14205260 A
C); (18271 14205279 G A); (18272 14205491 A G); (18273 14205757 T C); (18274 14208579 G T); (18275
14209111 G A); (18276 14210322 A T); (18277 14210713 G A); (18278 14210848 G T); (18279 14212809
T C); (18280 14214838 C T); (18281 14214920 G A); (18282 14216482 C T); (18283 14217164 T G); (18284
14218038 T C); (18285 14218056 C T); (18286 14218962 G C); (18287 14219473 T C); (18288 14219670 A
G); (18289 14219676 A G); (18290 14219728 G T); (18291 14220036 T C); (18292 14220398 T C); (18293
14220543 C T); (18294 14220594 T C); (18295 14220967 G A); (18296 14221528 G C); (18297 14221793
C CTGGCGCGGA); (18298 14222619 C T); (18299 14223188 C T); (18300 14223497 A G); (18301
14223627 C T); (18302 14225185 A G); (18303 14225567 C T); (18304 14225621 A T); (18305 14226402 T
C); (18306 14227225 T C); (18307 14227681 A T); (18308 14227769 T C); (18309 14228395 G A); (18310
14228802 T C); (18311 14229452 G A); (18312 14230259 T C); (18313 14230796 A G); (18314 14231580
GA); (18315 14233088 G A); (18316 14233536 C T); (18317 14233922 G A); (18318 14234416 A
C); (18319 14236155 C T); (18320 14236579 G A); (18321 14236691 G A); (18322 14237025 T C); (18323
14238014 T C); (18324 14238318 GA G); (18325 14238485 T C); (18326 14238609 C G); (18327
14239294 C T); (18328 14240016 C T); (18329 14240043 A G); (18330 14240199 C T); (18331 14240452
G A); (18332 14241097 T A); (18333 14241263 C T); (18334 14242203 A G); (18335 14242470 G
A); (18336 14242625 G A); (18337 14243238 G C); (18338 14243909 T C); (18339 14244286 T C); (18340
14244469 C G); (18341 14244927 C T); (18342 14246043 TA T); (18343 14246130 C T); (18344 14247205
T G); (18345 14247238 C T); (18346 14247464 G T); (18347 14247513 G T); (18348 14248356 T C); (18349
14248468 T G); (18350 14248526 T C); (18351 14248683 T C); (18352 14249449 C T); (18353 14250939
G A); (18354 14251242 A G); (18355 14251454 C G); (18356 14252253 C T); (18357 14252278 C
T); (18358 14252328 C T); (18359 14252358 C T); (18360 14252377 C T); (18361 14252380 A G); (18362
14253457 T G); (18363 14253768 G T); (18364 14254252 T C); (18365 14255007 G A); (18366 14255037
T C); (18367 14255389 T C); (18368 14255606 A G); (18369 14255610 C T); (18370 14256907 G
A); (18371 14257197 C T); (18372 14257327 C T); (18373 14257342 T C); (18374 14257387 A C); (18375
14257842 G A); (18376 14257856 A G); (18377 14257917 C T); (18378 14258051 C CA); (18379
14261756 T G); (18380 14263592 T G); (18381 14264041 T TA); (18382 14264619 C CTTTTCTAG); (18383
14266171 C T); (18384 14266255 G T); (18385 14267176 G A); (18386 14267206 C T); (18387 14267657
A G); (18388 14267786 A G); (18389 14268025 T C); (18390 14268452 G A); (18391 14268847 G
A); (18392 14268894 G A); (18393 14269264 C G); (18394 14269798 G A); (18395 14269800 G A); (18396
14269855 G A); (18397 14269922 T C); (18398 14270155 C T); (18399 14270392 T C); (18400 14271086 T
C); (18401 14271246 G C); (18402 14271250 T C); (18403 14271275 A C); (18404 14271415 T G); (18405
14273899 G A); (18406 14274304 C A); (18407 14274574 C T); (18408 14274719 T C); (18409 14274973
A T); (18410 14275045 A G); (18411 14275593 A T); (18412 14275689 T A); (18413 14275882 G
A); (18414 14276111 A G); (18415 14277599 C T); (18416 14277994 C T); (18417 14278287 T C); (18418
14278334 G A); (18419 14278695 C T); (18420 14279139 G A); (18421 14280198 C T); (18422 14280434
A T); (18423 14282071 G T); (18424 14282452 G T); (18425 14284300 A T); (18426 14284309 G
A); (18427 14284504 G T); (18428 14286023 G A); (18429 14286379 A G); (18430 14286677 C T); (18431
14286957 A G); (18432 14290261 C T); (18433 14292800 C A); (18434 14293004 T C); (18435 14293006
A G); (18436 14293286 C T); (18437 14294003 A G); (18438 14295462 G A); (18439 14295896 G
A); (18440 14296026 T C); (18441 14296177 T C); (18442 14296390 A G); (18443 14297387 A G); (18444
14297746 G A); (18445 14297757 G A); (18446 14297836 A G); (18447 14298402 C T); (18448 14298657
T C); (18449 14299622 A C); (18450 14300050 C A); (18451 14300468 G A); (18452 14300563 G
A); (18453 14300893 T C); (18454 14301162 C T); (18455 14302125 A G); (18456 14302245 T C); (18457
14303056 A C); (18458 14303492 G A); (18459 14304689 T C); (18460 14304896 A G); (18461 14305191
A G); (18462 14305549 A G); (18463 14305574 G C); (18464 14305929 A T); (18465 14307236 T
C); (18466 14307332 G A); (18467 14307480 C T); (18468 14308151 T C); (18469 14308156 G A); (18470
14308889 A G); (18471 14309053 T C); (18472 14309393 G T); (18473 14309689 A T); (18474 14309797
A G); (18475 14310045 C G); (18476 14310653 A T); (18477 14310758 G A); (18478 14311717 T
C); (18479 14312175 C T); (18480 14312659 C T); (18481 14313186 C T); (18482 14313940 G A); (18483
14314099 G A); (18484 14314318 C T); (18485 14314972 T G); (18486 14316132 A G); (18487 14316214
G A); (18488 14316351 G T); (18489 14316525 A T); (18490 14316814 G A); (18491 14317223 T
C); (18492 14318025 C T); (18493 14318576 C T); (18494 14318622 ACTACCCT A); (18495 14319421 C
A); (18496 14319435 C T); (18497 14321162 T A); (18498 14321599 T C); (18499 14322623 C A); (18500
14322742 G A); (18501 14323436 T C); (18502 14324153 A G); (18503 14325276 G A); (18504 14326137
A T); (18505 14326279 G A); (18506 14326337 A G); (18507 14326382 T C); (18508 14326822 T
C); (18509 14327110 T A); (18510 14327640 G A); (18511 14328122 A G); (18512 14328253 T C); (18513
14328262 A G); (18514 14328802 A G); (18515 14329307 G A); (18516 14329737 G T); (18517 14329771
C T); (18518 14331105 G A); (18519 14331647 T A); (18520 14332086 T C); (18521 14332486 A
G); (18522 14332902 G A); (18523 14333234 G C); (18524 14333640 A T); (18525 14333664 G T); (18526
14333834 A G); (18527 14334311 T G); (18528 14334628 T C); (18529 14335340 G A); (18530 14336075
C T); (18531 14337855 T A); (18532 14338160 C T); (18533 14338392 G T); (18534 14338631 A G); (18535
14339819 G C); (18536 14339946 C T); (18537 14340463 T C); (18538 14340485 C T); (18539 14340668
TA T); (18540 14340799 A G); (18541 14340907 T A); (18542 14340924 A C); (18543 14341008 C
G); (18544 14341045 G A); (18545 14341084 T C); (18546 14341296 AGTGAATG A); (18547 14341821 G
A); (18548 14342514 G A); (18549 14342681 G A); (18550 14343153 C T); (18551 14343562 G A); (18552
14343812 C T); (18553 14344266 C T); (18554 14344680 T C); (18555 14345047 G A); (18556 14345364 C
T); (18557 14345523 A G); (18558 14346004 C T); (18559 14346484 C T); (18560 14347699 C T); (18561
14347700 C T); (18562 14347991 C T); (18563 14348317 T C); (18564 14348656 T C); (18565 14348935 C
T); (18566 14349839 G A); (18567 14350166 A T); (18568 14350174 C T); (18569 14350573 C T); (18570
14350705 T G); (18571 14350707 A T); (18572 14350760 T C); (18573 14351324 A G); (18574 14351830
A G); (18575 14352342 C T); (18576 14352496 T A); (18577 14352768 T A); (18578 14352771 A T); (18579
14354710 G A); (18580 14356278 G A); (18581 14356374 T C); (18582 14357851 G A); (18583 14358098
T C); (18584 14358335 C G); (18585 14358526 C A); (18586 14359670 A G); (18587 14360588 G
A); (18588 14360603 A G); (18589 14360706 G A); (18590 14360780 G A); (18591 14360897 A C); (18592
14360903 A C); (18593 14361180 A C); (18594 14361266 C T); (18595 14361926 C T); (18596 14362001 T
C); (18597 14362566 T C); (18598 14364423 A G); (18599 14364539 C T); (18600 14364845 G A); (18601
14365144 T C); (18602 14365646 C A); (18603 14366303 G C); (18604 14366311 A T); (18605 14367525
TG T); (18606 14368458 T C); (18607 14368571 C T); (18608 14368774 C A); (18609 14368990 A
G); (18610 14369065 T C); (18611 14369556 T C); (18612 14369574 C T); (18613 14370028 A G); (18614
14370401 C T); (18615 14370435 A G); (18616 14370764 G A); (18617 14371315 C T); (18618 14371520
T A); (18619 14372693 C T); (18620 14373031 A C); (18621 14373538 T C); (18622 14373698 C T); (18623
14374001 G A); (18624 14374221 A G); (18625 14374413 G A); (18626 14375002 A G); (18627 14375464
A C); (18628 14375980 A G); (18629 14376170 T G); (18630 14376479 C A); (18631 14376650 C
A); (18632 14377458 C G); (18633 14378923 A C); (18634 14379104 C T); (18635 14379723 G A); (18636
14379941 A C); (18637 14380524 C T); (18638 14381045 T C); (18639 14381046 C T); (18640 14381590 T
G); (18641 14381892 C T); (18642 14381990 A G); (18643 14382254 C T); (18644 14384459 T C); (18645
14384714 T C); (18646 14385714 C T); (18647 14385851 G A); (18648 14385961 C G); (18649 14386162
G A); (18650 14386250 G A); (18651 14386879 C A); (18652 14387038 G A); (18653 14387055 T
TG); (18654 14387432 T C); (18655 14387511 T G); (18656 14387991 T C); (18657 14388180 C T); (18658
14388184 A C); (18659 14388248 G A); (18660 14388274 C A); (18661 14388335 G T); (18662 14389109
C A); (18663 14391220 AT A); (18664 14393788 A C); (18665 14393857 G GT); (18666 14393860 C
T); (18667 14394066 A G); (18668 14401828 T G); (18669 14401829 C T); (18670 14401941 G C); (18671
14402910 T C); (18672 14402985 G A); (18673 14403141 C T); (18674 14403577 G A); (18675 14403729
T C); (18676 14403759 A T); (18677 14404291 G A); (18678 14404643 C T); (18679 14404863 C T); (18680
14405131 G A); (18681 14405243 G T); (18682 14405345 T C); (18683 14405496 G A); (18684 14405532
A G); (18685 14405824 A G); (18686 14406205 C T); (18687 14406310 C G); (18688 14406444 T
C); (18689 14406600 T C); (18690 14406669 TG T); (18691 14407999 A G); (18692 14408085 T A); (18693
14408326 G C); (18694 14408765 G A); (18695 14410492 G T); (18696 14410665 C G); (18697 14411308
C T); (18698 14411873 G A); (18699 14411932 G A); (18700 14412463 A C); (18701 14414304 G
T); (18702 14415160 A T); (18703 14415434 A G); (18704 14416020 C T); (18705 14417178 C A); (18706
14417414 A G); (18707 14418429 T G); (18708 14419857 A AT); (18709 14419968 C A); (18710
14420860 G A); (18711 14421004 C T); (18712 14421227 C T); (18713 14421586 C T); (18714 14421589
G A); (18715 14421639 T G); (18716 14421695 T C); (18717 14421978 A G); (18718 14422030 T
C); (18719 14422464 G A); (18720 14423120 T G); (18721 14423579 G C); (18722 14423852 C A); (18723
14425078 A T); (18724 14425709 C T); (18725 14426059 TCTA T); (18726 14426649 A C); (18727
14427278 A G); (18728 14427872 A T); (18729 14428124 A G); (18730 14428402 G A); (18731 14428504
C T); (18732 14429145 T C); (18733 14429148 T C); (18734 14430456 T C); (18735 14431153 A C); (18736
14431554 A T); (18737 14431688 A T); (18738 14432136 C T); (18739 14432180 G A); (18740 14432372
T C); (18741 14432710 G A); (18742 14433306 T TA); (18743 14433678 G C); (18744 14434444 T
C); (18745 14434720 G T); (18746 14434935 A G); (18747 14435191 A AT); (18748 14435453 A
G); (18749 14436044 G A); (18750 14436345 T TA); (18751 14436472 T C); (18752 14437160 C G); (18753
14437554 G T); (18754 14438293 C T); (18755 14438512 G A); (18756 14439005 T C); (18757 14440123
A G); (18758 14440246 T C); (18759 14440699 G A); (18760 14440909 T A); (18761 14441481 A
G); (18762 14441490 T A); (18763 14442017 G A); (18764 14442361 C G); (18765 14443333 A G); (18766
14444177 A C); (18767 14445477 A G); (18768 14445518 GT G); (18769 14445895 A AT); (18770
14446678 AT A); (18771 14448394 GT G); (18772 14448619 T A); (18773 14448699 A G); (18774
14449846 C T); (18775 14450218 T C); (18776 14450252 C T); (18777 14450812 T C); (18778 14451624 C
T); (18779 14453085 C A); (18780 14453703 A C); (18781 14454300 T A); (18782 14454335 G A); (18783
14455492 T G); (18784 14455985 A G); (18785 14456050 C T); (18786 14457132 G A); (18787 14457433
T A); (18788 14458407 A AT); (18789 14458716 C T); (18790 14458963 G A); (18791 14459609 C
T); (18792 14459616 C T); (18793 14459682 C T); (18794 14459814 T C); (18795 14459888 A G); (18796
14460805 G A); (18797 14461158 A T); (18798 14461237 T TA); (18799 14461439 A G); (18800
14461517 G A); (18801 14461570 C CA); (18802 14461681 A T); (18803 14463807 TA T); (18804
14463942 C T); (18805 14463991 G A); (18806 14464441 A T); (18807 14465543 G A); (18808 14465851
T G); (18809 14466881 A C); (18810 14467764 T C); (18811 14467828 T TTATA); (18812 14468429 C
A); (18813 14470342 T C); (18814 14471453 T C); (18815 14475122 T A); (18816 14475346 C G); (18817
14476661 A G); (18818 14476833 T G); (18819 14477439 TTTTTTTCGGTTTTCAA T); (18820 14477927 C
T); (18821 14477997 T C); (18822 14481086 A T); (18823 14481143 A G); (18824 14481521 G A); (18825
14482408 G A); (18826 14482774 G T); (18827 14483921 T G); (18828 14485529 G A); (18829 14486372
T G); (18830 14486610 T C); (18831 14487005 A G); (18832 14487684 C T); (18833 14487904 A
C); (18834 14488403 C G); (18835 14489833 A G); (18836 14489856 A G); (18837 14490411 G A); (18838
14490606 T TTA); (18839 14491343 A C); (18840 14491739 G A); (18841 14492070 T C); (18842
14492134 C T); (18843 14492260 G A); (18844 14492339 G A); (18845 14493178 T C); (18846 14493797
A C); (18847 14493923 G T); (18848 14494131 T G); (18849 14495211 C T); (18850 14496238 G
A); (18851 14496380 G A); (18852 14496551 G A); (18853 14497099 G A); (18854 14497127 T C); (18855
14497581 C A); (18856 14498414 C T); (18857 14498439 A G); (18858 14498520 T C); (18859 14499043
G C); (18860 14499670 T A); (18861 14501249 T TC); (18862 14501500 C T); (18863 14501735 G
A); (18864 14504035 G A); (18865 14506300 A G); (18866 14507571 C T); (18867 14508035 G A); (18868
14508683 A G); (18869 14508903 A G); (18870 14509250 C T); (18871 14509497 C T); (18872 14509625
G A); (18873 14510042 G C); (18874 14510087 T C); (18875 14511220 C A); (18876 14511917 G
A); (18877 14513269 C A); (18878 14513758 C T); (18879 14513956 G A); (18880 14515756 A G); (18881
14516887 G C); (18882 14516888 A G); (18883 14516889 A G); (18884 14516929 C T); (18885 14517018
CG C); (18886 14517070 C T); (18887 14517073 T C); (18888 14517114 G A); (18889 14517450 T
C); (18890 14517785 T C); (18891 14517941 A G); (18892 14518142 A G); (18893 14518426 C A); (18894
14518798 T C); (18895 14519783 T G); (18896 14519873 G T); (18897 14520007 A G); (18898 14520700
C T); (18899 14520717 T A); (18900 14521077 A T); (18901 14521602 G A); (18902 14522446 T C); (18903
14526213 G A); (18904 14526344 C T); (18905 14526676 G T); (18906 14527038 G A); (18907 14527041
G A); (18908 14527194 A G); (18909 14527298 G A); (18910 14527736 C T); (18911 14527792 C
T); (18912 14528098 G A); (18913 14528949 C T); (18914 14529057 A G); (18915 14530136 G T); (18916
14530275 G A); (18917 14530725 A G); (18918 14531868 A T); (18919 14532321 C T); (18920 14532758
A G); (18921 14533034 A C); (18922 14533391 G C); (18923 14534385 G A); (18924 14534894 T
G); (18925 14535059 A G); (18926 14535245 G A); (18927 14535346 C T); (18928 14537243 A G); (18929
14537457 C T); (18930 14537686 G A); (18931 14538870 T C); (18932 14539011 G A); (18933 14539025
A G); (18934 14539695 C A); (18935 14539753 C T); (18936 14539970 C T); (18937 14540239 G
A); (18938 14540986 A G); (18939 14541841 GA G); (18940 14542665 A G); (18941 14543206 A
C); (18942 14545588 AT A); (18943 14545634 G A); (18944 14547066 G T); (18945 14547205 T A); (18946
14548137 G A); (18947 14548777 G A); (18948 14548824 C A); (18949 14549844 C T); (18950 14549857
G A); (18951 14549866 G A); (18952 14550414 T G); (18953 14551511 C T); (18954 14554313 T
G); (18955 14554527 G A); (18956 14554584 T A); (18957 14554816 C T); (18958 14555458 A T); (18959
14555963 T TA); (18960 14556272 CA C); (18961 14557512 T C); (18962 14558126 A G); (18963
14558283 A G); (18964 14558421 C T); (18965 14559162 T G); (18966 14559891 G T); (18967 14560183
G A); (18968 14560322 G A); (18969 14560396 T A); (18970 14560474 TA T); (18971 14561373 TA
T); (18972 14567603 A G); (18973 14568617 C T); (18974 14569561 A C); (18975 14569662 C A); (18976
14570020 G A); (18977 14570284 T C); (18978 14570758 C T); (18979 14570893 C T); (18980 14571002
A G); (18981 14571436 C T); (18982 14571535 C T); (18983 14571747 G A); (18984 14572085 T
C); (18985 14573188 T C); (18986 14573336 C G); (18987 14573599 A G); (18988 14573864 G A); (18989
14574511 G T); (18990 14574727 G T); (18991 14574997 G A); (18992 14575383 A G); (18993 14575626
C T); (18994 14579187 C T); (18995 14580759 G T); (18996 14581220 A C); (18997 14581911 G
A); (18998 14582083 A C); (18999 14582137 G C); (19000 14583477 C A); (19001 14584321 A G); (19002
14584419 C A); (19003 14585800 A C); (19004 14587428 A G); (19005 14588479 C G); (19006 14588708
AT A); (19007 14589989 G A); (19008 14590688 T C); (19009 14591102 G A); (19010 14591124 G
T); (19011 14591248 T C); (19012 14591421 G A); (19013 14591518 T C); (19014 14591852 A G); (19015
14591930 C T); (19016 14592023 C T); (19017 14593554 C G); (19018 14593607 C A); (19019 14593802
G A); (19020 14594159 C T); (19021 14594307 T G); (19022 14594633 G A); (19023 14594908 G
A); (19024 14594919 C T); (19025 14594927 A T); (19026 14595297 G A); (19027 14595690 G C); (19028
14595694 AAC A); (19029 14595907 T C); (19030 14596060 A G); (19031 14596289 C T); (19032
14596421 C G); (19033 14596855 C T); (19034 14596888 G A); (19035 14597433 T C); (19036 14597554
T C); (19037 14597634 C T); (19038 14597848 A G); (19039 14597929 C G); (19040 14597995 A
C); (19041 14600362 G A); (19042 14600500 G C); (19043 14601186 A G); (19044 14602530 G T); (19045
14603216 C A); (19046 14603422 TA T); (19047 14603588 G T); (19048 14605592 C T); (19049 14605602
G A); (19050 14606139 T C); (19051 14606504 T C); (19052 14607429 T C); (19053 14607603 T G); (19054
14607720 T C); (19055 14608932 A G); (19056 14609092 C T); (19057 14609307 T C); (19058 14609492
G T); (19059 14609502 C T); (19060 14609553 G T); (19061 14610755 T C); (19062 14611823 C A); (19063
14612987 T C); (19064 14613415 C A); (19065 14614348 T C); (19066 14614522 T C); (19067 14614939
GTATTTT G); (19068 14615730 T A); (19069 14616583 G C); (19070 14616633 G A); (19071 14617632 G
A); (19072 14617802 G C); (19073 14617906 A G); (19074 14618548 G C); (19075 14618692 C T); (19076
14619033 A G); (19077 14619075 C T); (19078 14620667 T G); (19079 14621897 A C); (19080 14622001
A C); (19081 14623556 T C); (19082 14623663 TAC T); (19083 14623677 C T); (19084 14623706 A
G); (19085 14623709 A G); (19086 14625976 C T); (19087 14626763 T C); (19088 14626790 G A); (19089
14627122 C T); (19090 14628134 G A); (19091 14630313 T C); (19092 14631336 C G); (19093 14631629
T C); (19094 14631774 A G); (19095 14632196 A G); (19096 14632507 T C); (19097 14632921 A
G); (19098 14633574 C T); (19099 14633653 C T); (19100 14634042 G A); (19101 14635100 T A); (19102
14635174 A G); (19103 14635693 G A); (19104 14635861 A G); (19105 14636010 A G); (19106 14636671
A C); (19107 14636751 C T); (19108 14637876 C T); (19109 14638614 G A); (19110 14640088 C T); (19111
14640206 A T); (19112 14640511 T A); (19113 14641573 A C); (19114 14642754 G A); (19115 14642758
T G); (19116 14642953 C T); (19117 14642973 A G); (19118 14645439 T A); (19119 14646081 A
G); (19120 14648267 A C); (19121 14649919 T C); (19122 14649944 A G); (19123 14650604 A G); (19124
14650949 T C); (19125 14651322 A T); (19126 14651341 G GA); (19127 14651398 C T); (19128 14652275
T A); (19129 14652470 G A); (19130 14652852 A AT); (19131 14654414 C T); (19132 14654916 A
G); (19133 14655217 C G); (19134 14656608 T C); (19135 14656855 A G); (19136 14656949 A G); (19137
14658133 C T); (19138 14659148 T A); (19139 14659358 C A); (19140 14659754 C T); (19141 14660454
G A); (19142 14661491 T C); (19143 14661761 A T); (19144 14661828 G A); (19145 14661835 A
G); (19146 14661844 C A); (19147 14661861 G A); (19148 14662360 A G); (19149 14662692 A G); (19150
14663036 T C); (19151 14663867 A T); (19152 14664302 A G); (19153 14664547 C T); (19154 14664610
C T); (19155 14665766 A C); (19156 14666641 A G); (19157 14668137 T C); (19158 14669494 T C); (19159
14669750 A G); (19160 14669883 A T); (19161 14670572 C T); (19162 14671633 C T); (19163 14671796 T
G); (19164 14671812 TCA T); (19165 14671845 C T); (19166 14671904 A G); (19167 14672391 C
A); (19168 14673605 G A); (19169 14674107 G A); (19170 14674658 T C); (19171 14674885 A G); (19172
14675473 A G); (19173 14675515 T C); (19174 14675729 C T); (19175 14675825 C T); (19176 14675872
A G); (19177 14675945 C G); (19178 14676125 C G); (19179 14677975 C A); (19180 14678910 T
G); (19181 14679479 C T); (19182 14679678 A G); (19183 14680165 G T); (19184 14680485 T
TAC); (19185 14680507 T C); (19186 14680584 T C); (19187 14681276 C A); (19188 14681732 G
A); (19189 14682023 T A); (19190 14682358 A G); (19191 14682376 C A); (19192 14683069 C T); (19193
14683072 G A); (19194 14685014 C T); (19195 14685305 T A); (19196 14685404 ACTT A); (19197
14685787 T G); (19198 14686558 G A); (19199 14686687 C G); (19200 14686830 T C); (19201 14687094
A G); (19202 14687387 T C); (19203 14688303 C T); (19204 14688515 C A); (19205 14688550 TA
T); (19206 14689261 T G); (19207 14689307 C T); (19208 14690155 C T); (19209 14690655 G
GGCTA); (19210 14691066 C T); (19211 14691328 A T); (19212 14691398 G A); (19213 14691402 A
G); (19214 14692082 T C); (19215 14692172 C T); (19216 14693558 G A); (19217 14694183 C T); (19218
14694538 T C); (19219 14694830 C A); (19220 14695585 A G); (19221 14697251 C T); (19222 14697491
A G); (19223 14697630 G A); (19224 14697653 G A); (19225 14697917 T C); (19226 14698078 C
T); (19227 14698523 C T); (19228 14698548 T G); (19229 14699380 A G); (19230 14700385 G A); (19231
14700853 G A); (19232 14700974 A G); (19233 14701379 T A); (19234 14701759 A C); (19235 14702078
T C); (19236 14702245 G A); (19237 14702810 G A); (19238 14703702 G A); (19239 14704271 G
A); (19240 14705342 T C); (19241 14705653 G A); (19242 14706580 T C); (19243 14707003 A G); (19244
14707468 C T); (19245 14708196 T G); (19246 14708366 A C); (19247 14708551 T C); (19248 14708767 T
C); (19249 14718501 T C); (19250 14718580 C T); (19251 14720106 G A); (19252 14721999 C T); (19253
14722031 C T); (19254 14722568 A G); (19255 14722970 C A); (19256 14722971 G A); (19257 14723012
C T); (19258 14723275 G A); (19259 14723276 T C); (19260 14723280 C A); (19261 14723287 G
A); (19262 14723502 G A); (19263 14723817 C T); (19264 14723853 C G); (19265 14723860 T G); (19266
14723861 G A); (19267 14723868 G A); (19268 14723929 G A); (19269 14728174 A G); (19270 14728657
G A); (19271 14728896 T G); (19272 14730387 T A); (19273 14731016 T C); (19274 14731778 G
A); (19275 14731834 G A); (19276 14732088 C T); (19277 14732221 T G); (19278 14733162 T
TGGTTGGGGTTTAGG); (19279 14734539 G A); (19280 14735086 T C); (19281 14736279 C T); (19282
14736492 A AT); (19283 14736781 A G); (19284 14737762 T A); (19285 14738081 C T); (19286 14739400
A C); (19287 14739406 G A); (19288 14740038 C T); (19289 14740197 C A); (19290 14740326 C
T); (19291 14740498 A G); (19292 14741583 T C); (19293 14744490 G A); (19294 14745087 G A); (19295
14745105 T C); (19296 14745587 A G); (19297 14746746 T C); (19298 14748522 A G); (19299 14748926
C T); (19300 14750950 G T); (19301 14751298 G T); (19302 14751753 T A); (19303 14752345 C A); (19304
14753789 G A); (19305 14754200 T C); (19306 14755222 G A); (19307 14755362 T
TGTGGCGCCAGGGGCGCCAGA); (19308 14755399 C T); (19309 14755559 A C); (19310 14755569 C
T); (19311 14755608 A G); (19312 14756512 T C); (19313 14756603 G A); (19314 14757947 A C); (19315
14757993 T C); (19316 14758200 C T); (19317 14758936 T C); (19318 14759005 C T); (19319 14759084 G
A); (19320 14759085 A C); (19321 14760139 C T); (19322 14760541 T C); (19323 14760879 C A); (19324
14761262 A G); (19325 14761832 A C); (19326 14761849 G A); (19327 14761879 A G); (19328 14763816
G A); (19329 14766039 C T); (19330 14766085 C T); (19331 14766949 T C); (19332 14767775 G C); (19333
14768406 A G); (19334 14769463 T C); (19335 14770200 A G); (19336 14770599 C T); (19337 14771251
A C); (19338 14771519 C T); (19339 14771567 T C); (19340 14771878 C T); (19341 14772001 T A); (19342
14773167 C T); (19343 14773295 C T); (19344 14774402 C T); (19345 14774691 G A); (19346 14775351
A T); (19347 14777316 C T); (19348 14778357 C T); (19349 14778365 G A); (19350 14778611 C T); (19351
14778765 T C); (19352 14778812 T C); (19353 14778900 C T); (19354 14779729 C CT); (19355 14780098
T G); (19356 14780460 C A); (19357 14780637 GTAATCGATTAT G); (19358 14780796 C T); (19359
14780862 A C); (19360 14780880 A C); (19361 14781374 C T); (19362 14781705 G C); (19363 14782001
C T); (19364 14782129 C T); (19365 14782200 T C); (19366 14782526 T G); (19367 14783329 G A); (19368
14784228 C T); (19369 14784514 T C); (19370 14784818 G A); (19371 14785057 C T); (19372 14785614
G A); (19373 14786408 G T); (19374 14786868 T A); (19375 14786908 A ACATCGATTTTT); (19376
14787424 T C); (19377 14788176 C T); (19378 14788242 C T); (19379 14788512 C T); (19380 14788555 C
T); (19381 14788741 G A); (19382 14788963 T A); (19383 14789028 T C); (19384 14789240 CA C); (19385
14789258 A G); (19386 14789845 T C); (19387 14790185 T C); (19388 14790618 G T); (19389 14791339
G A); (19390 14791659 A C); (19391 14791750 C T); (19392 14792020 C T); (19393 14792123 T
TTGTTTTC); (19394 14792531 T C); (19395 14792632 C T); (19396 14792871 C A); (19397 14792905 G
A); (19398 14794481 A G); (19399 14794901 G T); (19400 14795042 T C); (19401 14795831 G A); (19402
14796602 T C); (19403 14797484 G A); (19404 14797939 G A); (19405 14798087 A C); (19406 14798132
A G); (19407 14798560 T G); (19408 14798739 A T); (19409 14799067 C T); (19410 14800439 A
T); (19411 14800868 G A); (19412 14800901 G A); (19413 14801292 C T); (19414 14801815 A G); (19415
14801955 C T); (19416 14802976 A G); (19417 14804032 T C); (19418 14804148 G A); (19419 14804153
C T); (19420 14805466 G GA); (19421 14805666 C A); (19422 14806137 AAT A); (19423 14807586 T
C); (19424 14807662 G C); (19425 14808097 A T); (19426 14808655 G A); (19427 14810503 T A); (19428
14810534 G A); (19429 14811014 G A); (19430 14811211 A AT); (19431 14811284 A G); (19432
14811499 G A); (19433 14811500 G A); (19434 14812620 T G); (19435 14813023 A C); (19436 14813617
T C); (19437 14814064 C T); (19438 14814596 C A); (19439 14814703 G A); (19440 14815698 G
A); (19441 14816466 G A); (19442 14816558 T C); (19443 14816613 G A); (19444 14816638 T C); (19445
14816665 G T); (19446 14816886 T C); (19447 14817005 A G); (19448 14817104 G A); (19449 14817361
T G); (19450 14818913 G T); (19451 14818973 C T); (19452 14819151 C T); (19453 14819854 T C); (19454
14820038 T C); (19455 14820643 C A); (19456 14821143 T C); (19457 14821230 A C); (19458 14822547 T
C); (19459 14823575 C CT); (19460 14823689 T C); (19461 14823959 A C); (19462 14825865 C
CT); (19463 14826845 TA T); (19464 14827233 G T); (19465 14827623 G A); (19466 14828761 A
AT); (19467 14829132 T C); (19468 14829334 C A); (19469 14829405 T C); (19470 14831024 G A); (19471
14831082 C T); (19472 14831531 T C); (19473 14831746 AT A); (19474 14833490 G A); (19475 14834166
CA C); (19476 14834581 C T); (19477 14834666 G A); (19478 14834850 C T); (19479 14835808 T
C); (19480 14836855 G A); (19481 14837199 T G); (19482 14837401 G T); (19483 14837882 A G); (19484
14838210 C T); (19485 14839180 G A); (19486 14839739 A C); (19487 14840119 G A); (19488 14842590
G A); (19489 14842842 C T); (19490 14842866 C T); (19491 14843452 A T); (19492 14843525 C T); (19493
14843891 G A); (19494 14844250 A T); (19495 14844540 G GT); (19496 14844675 A C); (19497
14845222 AT A); (19498 14845400 C T); (19499 14846462 G T); (19500 14846557 C T); (19501 14847073
C G); (19502 14847126 A G); (19503 14848331 T C); (19504 14848358 T A); (19505 14849053 C
T); (19506 14849186 T C); (19507 14849422 G A); (19508 14851301 G C); (19509 14852619 G T); (19510
14852891 G T); (19511 14854618 T C); (19512 14855719 A T); (19513 14856103 AT A); (19514 14856990
ATTT A); (19515 14857573 A G); (19516 14857647 A C); (19517 14857768 G A); (19518 14857784 A
G); (19519 14857809 A G); (19520 14858830 A G); (19521 14858928 T G); (19522 14859525 G A); (19523
14859641 C T); (19524 14859713 T TGCCCCAAGCGTGGGTTCCCAC); (19525 14860140 G A); (19526
14860580 G A); (19527 14860748 C T); (19528 14860917 A G); (19529 14860932 A G); (19530 14861468
C T); (19531 14862359 C T); (19532 14862467 G A); (19533 14862823 A T); (19534 14862956 T G); (19535
14863021 C T); (19536 14863368 C T); (19537 14864653 AT A); (19538 14864844 G C); (19539 14864994
G A); (19540 14865424 A G); (19541 14865437 A C); (19542 14865974 A T); (19543 14866167 G
A); (19544 14866584 C T); (19545 14868188 C T); (19546 14868367 C T); (19547 14869229 A C); (19548
14870003 A C); (19549 14871834 C T); (19550 14874279 T C); (19551 14874539 C T); (19552 14875536 T
C); (19553 14875906 A G); (19554 14875925 C A); (19555 14876284 G A); (19556 14876438 AG
A); (19557 14876858 A G); (19558 14877078 A T); (19559 14877307 C T); (19560 14877344 A G); (19561
14877443 A C); (19562 14878301 A G); (19563 14878371 TA T); (19564 14878814 T G); (19565 14878950
G GA); (19566 14881915 A T); (19567 14886646 C A); (19568 14886800 T C); (19569 14888046 C
T); (19570 14888561 T C); (19571 14889047 T C); (19572 14889831 G A); (19573 14890310 TTATATA
T); (19574 14890322 T C); (19575 14890592 A AC); (19576 14890853 T C); (19577 14890870
TGTCTATCATTATTA T); (19578 14890936 A G); (19579 14892045 C T); (19580 14892309 G C); (19581
14892499 T C); (19582 14893304 G C); (19583 14895139 G A); (19584 14895656 C T); (19585 14896156
T C); (19586 14896265 A G); (19587 14896467 G A); (19588 14896619 G C); (19589 14897455 A
G); (19590 14897525 A G); (19591 14897592 TTA T); (19592 14898335 T A); (19593 14898398 C
T); (19594 14898667 A T); (19595 14898958 A AT); (19596 14899004 T C); (19597 14899198 T C); (19598
14899511 G A); (19599 14899642 C T); (19600 14900016 C T); (19601 14900359 G A); (19602 14900371
G GT); (19603 14901855 G C); (19604 14902549 G A); (19605 14903228 C T); (19606 14903513 G
A); (19607 14904602 C T); (19608 14906341 C T); (19609 14906359 C T); (19610 14906470 G A); (19611
14906815 C T); (19612 14907423 C T); (19613 14909018 G A); (19614 14909309 G A); (19615 14909668
C T); (19616 14909920 G A); (19617 14910402 A AT); (19618 14910590 A AT); (19619 14910732 G
A); (19620 14911042 G A); (19621 14911384 T C); (19622 14911388 C T); (19623 14911825 C T); (19624
14912699 G T); (19625 14913277 A G); (19626 14913442 G A); (19627 14913741 C G); (19628 14914486
A G); (19629 14914564 T C); (19630 14914609 T C); (19631 14914876 C T); (19632 14915004 C A); (19633
14915133 A T); (19634 14915191 C T); (19635 14915606 C A); (19636 14916004 A G); (19637 14916339
A G); (19638 14916401 T A); (19639 14916699 A G); (19640 14917001 G A); (19641 14917369 A
G); (19642 14917433 C A); (19643 14918604 C T); (19644 14918657 C T); (19645 14919578 C T); (19646
14919596 G A); (19647 14921022 G C); (19648 14921582 G A); (19649 14921670 A G); (19650 14921885
T C); (19651 14921908 G A); (19652 14926626 G A); (19653 14927468 G A); (19654 14927626 T
C); (19655 14928317 C CT); (19656 14929646 C A); (19657 14929968 G A); (19658 14930530 T G); (19659
14931604 C CACAT); (19660 14933941 G A); (19661 14934120 G A); (19662 14934174 T C); (19663
14934878 C T); (19664 14934902 A G); (19665 14934915 G A); (19666 14934921 C T); (19667 14935161
T A); (19668 14935210 A G); (19669 14937426 T C); (19670 14938035 C T); (19671 14939023 C T); (19672
14939603 C T); (19673 14939622 A G); (19674 14939730 C T); (19675 14939976 A G); (19676 14941145
A G); (19677 14942472 T C); (19678 14942578 G A); (19679 14942719 G A); (19680 14944416 A
AT); (19681 14944853 C T); (19682 14945406 T C); (19683 14946046 T C); (19684 14946518 C T); (19685
14946540 C T); (19686 14946989 C CA); (19687 14947465 G A); (19688 14947512 A C); (19689 14947558
C T); (19690 14947606 T C); (19691 14947748 C T); (19692 14947757 T C); (19693 14947825 A G); (19694
14948348 T C); (19695 14949503 G A); (19696 14949733 C CAAG); (19697 14949853 T C); (19698
14949925 C T); (19699 14950192 T C); (19700 14950505 C A); (19701 14950996 C A); (19702 14951073
A G); (19703 14951183 C T); (19704 14951303 C T); (19705 14951473 T G); (19706 14952381 A
G); (19707 14952687 A G); (19708 14952926 A G); (19709 14953172 T G); (19710 14953510 C T); (19711
14953586 C T); (19712 14953794 CT C); (19713 14955579 C A); (19714 14956423 C T); (19715 14957294
G C); (19716 14957984 G A); (19717 14958397 A T); (19718 14958626 G T); (19719 14962131 A
G); (19720 14962640 T C); (19721 14962946 G A); (19722 14963196 A G); (19723 14963376 C T); (19724
14963428 T A); (19725 14963447 C T); (19726 14964045 T A); (19727 14964198 A G); (19728 14965031
C A); (19729 14965086 C T); (19730 14965442 G A); (19731 14965614 C T); (19732 14965826 G
A); (19733 14965885 C T); (19734 14967873 G T); (19735 14967940 C G); (19736 14968510 G A); (19737
14968538 G A); (19738 14968602 C T); (19739 14971600 T G); (19740 14971684 G T); (19741 14971906
A C); (19742 14972596 C T); (19743 14973738 G A); (19744 14974051 A G); (19745 14975830 G
A); (19746 14975957 T A); (19747 14976084 C T); (19748 14976105 G A); (19749 14976449 G A); (19750
14976659 C T); (19751 14977179 G A); (19752 14978161 G A); (19753 14979434 T C); (19754 14980205
A G); (19755 14980279 G C); (19756 14982473 A G); (19757 14982603 A C); (19758 14983418 A
G); (19759 14983951 C T); (19760 14984430 C T); (19761 14984488 G A); (19762 14984774 C A); (19763
14984808 C T); (19764 14985241 C A); (19765 14985344 G A); (19766 14986461 C T); (19767 14986514
C A); (19768 14986702 T C); (19769 14986735 G A); (19770 14986873 C T); (19771 14987132 A
C); (19772 14987271 T C); (19773 14987435 C T); (19774 14987466 G A); (19775 14988457 A G); (19776
14988491 T C); (19777 14988516 C T); (19778 14988535 T C); (19779 14988558 T A); (19780 14988879 G
A); (19781 14989591 G A); (19782 14990065 G A); (19783 14990253 A G); (19784 14992304 C A); (19785
14992662 G T); (19786 14993729 A G); (19787 14995600 C CA); (19788 14995964 G GTTAA); (19789
14996330 T C); (19790 14996332 A T); (19791 14996334 A T); (19792 14996878 G A); (19793 14996879
A C); (19794 14998432 C T); (19795 14998962 A G); (19796 14999940 G T); (19797 15001621 A
G); (19798 15002201 C G); (19799 15002243 G A); (19800 15002650 C T); (19801 15003262 C T); (19802
15003639 C T); (19803 15003967 G T); (19804 15005199 T C); (19805 15005432 G A); (19806 15005641
T C); (19807 15006169 T A); (19808 15006181 T A); (19809 15006199 C A); (19810 15006311 G
A); (19811 15006704 T G); (19812 15007200 C T); (19813 15007252 A G); (19814 15009388 C T); (19815
15009402 C T); (19816 15009417 T G); (19817 15009612 A T); (19818 15009756 A G); (19819 15010406
A AT); (19820 15011991 G T); (19821 15012788 A G); (19822 15012870 A G); (19823 15013932 G
A); (19824 15014389 T C); (19825 15014835 A G); (19826 15016095 G A); (19827 15016102 G A); (19828
15016126 T A); (19829 15016135 C T); (19830 15016144 T A); (19831 15016186 G A); (19832 15016246
G A); (19833 15016255 C T); (19834 15016377 C T); (19835 15016399 G A); (19836 15016409 C
T); (19837 15016432 C T); (19838 15016437 A G); (19839 15016443 A C); (19840 15016449 T C); (19841
15016477 C T); (19842 15016479 T G); (19843 15016528 A C); (19844 15016529 G T); (19845 15016531
C A); (19846 15016596 C T); (19847 15016931 G A); (19848 15017405 T G); (19849 15017632 G
A); (19850 15017971 G T); (19851 15018150 C T); (19852 15018177 C A); (19853 15018403 T C); (19854
15018408 C T); (19855 15018639 T C); (19856 15018646 C T); (19857 15019221 A G); (19858 15019965 T
A); (19859 15022502 G A); (19860 15024029 A T); (19861 15024608 C T); (19862 15025706 A G); (19863
15025967 T C); (19864 15026088 C T); (19865 15026306 G A); (19866 15026948 T C); (19867 15027220
G A); (19868 15031415 GT G); (19869 15034054 G A); (19870 15034072 A G); (19871 15034114 A
C); (19872 15034172 C T); (19873 15034207 G A); (19874 15034217 T C); (19875 15034288 T C); (19876
15034303 T A); (19877 15035134 G T); (19878 15035139 A G); (19879 15035164 C A); (19880 15035202
C T); (19881 15035698 C T); (19882 15035713 C A); (19883 15035722 T C); (19884 15036688 G A); (19885
15039398 A T); (19886 15039401 T A); (19887 15039475 T C); (19888 15043983 GT G); (19889 15045152
A G); (19890 15045161 C T); (19891 15045260 G A); (19892 15045265 A G); (19893 15045307 T
C); (19894 15045335 T C); (19895 15045347 T C); (19896 15045373 C A); (19897 15045375 A G); (19898
15045380 AG A); (19899 15046619 G A); (19900 15046679 A C); (19901 15046737 C T); (19902
15046772 G A); (19903 15046782 T C); (19904 15046789 A C); (19905 15047185 A G); (19906 15047219
A G); (19907 15047281 G A); (19908 15047288 A T); (19909 15047423 C T); (19910 15047483 C
T); (19911 15047504 A G); (19912 15049145 T A); (19913 15051595 A G); (19914 15053892 A G); (19915
15054550 A G); (19916 15055239 T C); (19917 15055788 G A); (19918 15055891 T C); (19919 15056020
A G); (19920 15057264 G T); (19921 15057625 C T); (19922 15057635 T C); (19923 15057713 G
C); (19924 15057799 A T); (19925 15059126 C T); (19926 15059292 C T); (19927 15059342 T C); (19928
15059364 G A); (19929 15059959 A C); (19930 15060038 T C); (19931 15060307 C A); (19932 15060818
T A); (19933 15061855 G T); (19934 15061924 T A); (19935 15061925 T C); (19936 15062864 G
A); (19937 15063620 C T); (19938 15063663 A C); (19939 15063896 G A); (19940 15064174 G A); (19941
15065101 A G); (19942 15065126 A T); (19943 15066288 A G); (19944 15066745 T TA); (19945
15067303 A G); (19946 15068936 C T); (19947 15069116 G A); (19948 15069316 T C); (19949 15069624
T G); (19950 15070244 G T); (19951 15071826 T A); (19952 15071872 T C); (19953 15073892 A C); (19954
15074726 C A); (19955 15075205 A C); (19956 15076549 T C); (19957 15076859 T C); (19958 15077070 C
A); (19959 15077432 G T); (19960 15077500 G A); (19961 15078176 G A); (19962 15078508 C T); (19963
15079615 C T); (19964 15080102 A C); (19965 15080186 A T); (19966 15080224 TAA T); (19967
15080527 A G); (19968 15080786 A G); (19969 15080809 T C); (19970 15081267 A T); (19971 15081728
A G); (19972 15081963 G A); (19973 15081970 AC A); (19974 15082200 A C); (19975 15083027 A
AC); (19976 15083088 A AT); (19977 15083524 A T); (19978 15085975 C T); (19979 15086507 T
C); (19980 15086858 C A); (19981 15087900 G A); (19982 15088359 T G); (19983 15088506 A G); (19984
15088596 A G); (19985 15089396 C T); (19986 15089398 C T); (19987 15089441 T C); (19988 15089710
G A); (19989 15090581 C T); (19990 15090904 G A); (19991 15092037 G T); (19992 15092322 G
T); (19993 15092983 A G); (19994 15093509 A G); (19995 15094314 G T); (19996 15095113 CATTTGT
C); (19997 15095991 T G); (19998 15096178 T C); (19999 15096557 T C); (20000 15097680 G A); (20001
15097786 G T); (20002 15097877 C G); (20003 15098083 T C); (20004 15099154 A C); (20005 15099226
T C); (20006 15099380 A G); (20007 15099456 G A); (20008 15100057 T TA); (20009 15100094 G
A); (20010 15100137 A G); (20011 15100861 T C); (20012 15101154 T C); (20013 15101613 C T); (20014
15102160 G A); (20015 15102806 A G); (20016 15102897 C T); (20017 15103094 G A); (20018 15103646
A G); (20019 15103776 A C); (20020 15104027 A G); (20021 15104071 G A); (20022 15104381 A
G); (20023 15104983 C T); (20024 15105008 G T); (20025 15105548 C T); (20026 15105838 C T); (20027
15106075 A C); (20028 15106102 A G); (20029 15107303 T A); (20030 15109162 G A); (20031 15109300
C G); (20032 15109463 G A); (20033 15111027 C G); (20034 15111615 C T); (20035 15113739 A
G); (20036 15113874 G A); (20037 15114536 T C); (20038 15114569 G A); (20039 15115764 C T); (20040
15117235 T C); (20041 15117261 G A); (20042 15117334 G A); (20043 15117509 A T); (20044 15118533
G T); (20045 15118761 G A); (20046 15119075 T A); (20047 15119409 T C); (20048 15119759 A
C); (20049 15120091 T G); (20050 15121066 C G); (20051 15121226 A AC); (20052 15121940 C T); (20053
15123858 C T); (20054 15124220 G A); (20055 15124699 C T); (20056 15124885 G A); (20057 15125052
G A); (20058 15125344 C T); (20059 15127321 A G); (20060 15127334 C A); (20061 15127347 C
G); (20062 15127478 C T); (20063 15127785 A G); (20064 15127911 T C); (20065 15129060 G A); (20066
15129395 T TTTTCAATTGTA); (20067 15129575 C T); (20068 15131105 G A); (20069 15131230 C
T); (20070 15131553 G A); (20071 15131865 G A); (20072 15131884 G C); (20073 15132392 G C); (20074
15132499 G A); (20075 15132900 A G); (20076 15132981 T C); (20077 15133016 C T); (20078 15133139
C T); (20079 15133148 C T); (20080 15133296 G A); (20081 15134032 A G); (20082 15134182 C
T); (20083 15135281 T C); (20084 15136975 T C); (20085 15138316 C T); (20086 15139428 A G); (20087
15141046 C G); (20088 15142030 G T); (20089 15142691 C A); (20090 15142736 G A); (20091 15143566
G A); (20092 15144031 G A); (20093 15144377 C T); (20094 15144476 C T); (20095 15144601 G
A); (20096 15144710 A G); (20097 15144768 T A); (20098 15144794 G A); (20099 15145836 G A); (20100
15146267 A G); (20101 15146537 T C); (20102 15147009 A ACT); (20103 15148385 T A); (20104
15149281 T C); (20105 15150305 G T); (20106 15150701 C T); (20107 15150872 C T); (20108 15151379
G A); (20109 15151498 G A); (20110 15151509 T C); (20111 15151748 A G); (20112 15152586 T
C); (20113 15152847 G A); (20114 15153064 A G); (20115 15155379 A G); (20116 15155896 C T); (20117
15155898 A T); (20118 15156090 C G); (20119 15156775 G A); (20120 15157368 C T); (20121 15157427
A G); (20122 15158212 C G); (20123 15158461 A G); (20124 15160204 A G); (20125 15160478 C
T); (20126 15160518 G A); (20127 15160738 G A); (20128 15160817 C T); (20129 15161117 G A); (20130
15161121 A T); (20131 15161123 A ATT); (20132 15161752 C T); (20133 15162020 A T); (20134
15164845 G A); (20135 15165012 G A); (20136 15165121 A G); (20137 15165481 C T); (20138 15166157
T C); (20139 15166183 A AT); (20140 15166293 G A); (20141 15169096 C T); (20142 15169670 A
AT); (20143 15169874 T TG); (20144 15170252 C G); (20145 15170254 T C); (20146 15170758 CA
C); (20147 15170898 C T); (20148 15171016 A G); (20149 15171050 A T); (20150 15171688 T C); (20151
15172109 G A); (20152 15175058 G A); (20153 15175103 C A); (20154 15175550 T G); (20155 15175610
C T); (20156 15175707 A G); (20157 15176290 C T); (20158 15176855 G A); (20159 15177054 TAATG
T); (20160 15177708 T G); (20161 15177748 T TA); (20162 15177910 G A); (20163 15177997 C T); (20164
15178411 C T); (20165 15178951 G A); (20166 15178961 C T); (20167 15180686 C A); (20168 15180913
C T); (20169 15181302 A G); (20170 15181692 G A); (20171 15181711 G C); (20172 15181964 C
T); (20173 15182618 A G); (20174 15182948 A T); (20175 15183086 G A); (20176 15183237 C T); (20177
15183436 G A); (20178 15183649 C T); (20179 15183693 A G); (20180 15183710 A T); (20181 15184016
G A); (20182 15184365 T C); (20183 15184810 C T); (20184 15185945 G A); (20185 15186000 T
G); (20186 15186388 T C); (20187 15186435 G A); (20188 15186460 G A); (20189 15186731 C G); (20190
15186894 T C); (20191 15186955 C T); (20192 15188008 A C); (20193 15188383 G A); (20194 15188928
C T); (20195 15188976 T C); (20196 15189793 G A); (20197 15190106 G A); (20198 15190875 A
G); (20199 15190992 G A); (20200 15191984 C T); (20201 15192495 G T); (20202 15193576 G A); (20203
15194883 C T); (20204 15194915 AT A); (20205 15194995 C A); (20206 15195410 A G); (20207 15195663
G A); (20208 15195973 C A); (20209 15196016 A G); (20210 15196090 G A); (20211 15196157 C
T); (20212 15196194 C T); (20213 15196285 G C); (20214 15196504 G A); (20215 15196526 C A); (20216
15196710 T C); (20217 15197242 C T); (20218 15197556 G T); (20219 15197720 G A); (20220 15198033
G A); (20221 15199095 A T); (20222 15199228 A G); (20223 15202345 C T); (20224 15204800 T
A); (20225 15206325 A G); (20226 15206890 G A); (20227 15207410 C A); (20228 15207448 G A); (20229
15207941 A G); (20230 15208424 G A); (20231 15208513 A C); (20232 15209813 AGG A); (20233
15210093 CT C); (20234 15210231 GA G); (20235 15210286 A AAAGTTTTAT); (20236 15210762 A
G); (20237 15211465 A G); (20238 15212929 G A); (20239 15212950 A G); (20240 15213332 A C); (20241
15213523 A G); (20242 15213528 A G); (20243 15214037 C G); (20244 15214154 A G); (20245 15214544
C T); (20246 15216136 G A); (20247 15216446 T G); (20248 15216476 G A); (20249 15216705 A
C); (20250 15218194 G A); (20251 15218934 T C); (20252 15219834 G A); (20253 15220102 C T); (20254
15220294 A G); (20255 15222058 T G); (20256 15223752 G A); (20257 15223757 G T); (20258 15223943
A G); (20259 15224000 T C); (20260 15224434 A T); (20261 15225213 C A); (20262 15225264 T C); (20263
15225784 A T); (20264 15225953 T C); (20265 15226081 T C); (20266 15227885 C A); (20267 15228070 C
T); (20268 15230546 T C); (20269 15231666 T G); (20270 15231998 G T); (20271 15234279 C T); (20272
15235082 C T); (20273 15235219 A T); (20274 15238307 TTTAATAAG T); (20275 15238322 G A); (20276
15238812 G A); (20277 15239909 C G); (20278 15240163 C CA); (20279 15241867 CAAAATATG
C); (20280 15244483 T A); (20281 15246416 C T); (20282 15246628 C G); (20283 15247706 T C); (20284
15247886 A G); (20285 15247935 T A); (20286 15248086 T C); (20287 15248260 C T); (20288 15248332
G T); (20289 15249139 T C); (20290 15250032 A G); (20291 15252143 G A); (20292 15252838 A
G); (20293 15256093 G A); (20294 15256530 T C); (20295 15256689 C T); (20296 15256758 T TC); (20297
15257309 C T); (20298 15257554 TC T); (20299 15257679 G A); (20300 15258176 A G); (20301 15258860
A G); (20302 15259319 T C); (20303 15260033 AT A); (20304 15260430 T TA); (20305 15261098 A
G); (20306 15261222 C T); (20307 15261536 T C); (20308 15261746 G C); (20309 15262907 G A); (20310
15263053 C A); (20311 15264656 A T); (20312 15265033 A C); (20313 15265868 GAAGCGGAGCA
G); (20314 15266292 C T); (20315 15266708 A C); (20316 15266813 C T); (20317 15266818 T C); (20318
15266945 C T); (20319 15266947 C T); (20320 15267112 T G); (20321 15267186 A G); (20322 15267414
C T); (20323 15267728 C T); (20324 15267733 C T); (20325 15267762 A G); (20326 15267784 C T); (20327
15267842 T C); (20328 15267843 G A); (20329 15268132 T G); (20330 15269359 G A); (20331 15270039
T G); (20332 15271822 T TACCTCCG); (20333 15271864 T C); (20334 15271909 G A); (20335 15271912 T
G); (20336 15272013 C A); (20337 15272284 G A); (20338 15273073 T TTATA); (20339 15274071 A
G); (20340 15274403 A C); (20341 15274688 T C); (20342 15275483 G A); (20343 15275611 T C); (20344
15275661 G A); (20345 15276054 G A); (20346 15276547 T C); (20347 15276848 A G); (20348 15276854
A G); (20349 15276855 T C); (20350 15276890 G A); (20351 15276945 C T); (20352 15277234 A
G); (20353 15277452 A C); (20354 15277642 AC A); (20355 15277746 G T); (20356 15278216 T C); (20357
15278341 A G); (20358 15278500 G A); (20359 15278642 T C); (20360 15278797 C T); (20361 15278982
A C); (20362 15279125 T C); (20363 15279383 C A); (20364 15279758 G A); (20365 15279872 C
T); (20366 15280088 C T); (20367 15281153 C A); (20368 15281231 T C); (20369 15281382 A G); (20370
15281525 C A); (20371 15281971 G A); (20372 15282028 A G); (20373 15282098 A G); (20374 15282305
T C); (20375 15283280 C T); (20376 15283493 C T); (20377 15283803 T G); (20378 15284806 T
TA); (20379 15285082 T C); (20380 15285957 C T); (20381 15286080 T C); (20382 15286192 G A); (20383
15286414 G T); (20384 15286513 C T); (20385 15287274 C T); (20386 15287526 A C); (20387 15287604
A G); (20388 15288009 T G); (20389 15288467 AC A); (20390 15289052 C T); (20391 15289180 C
G); (20392 15289192 G A); (20393 15289295 A T); (20394 15290432 C T); (20395 15291570 C T); (20396
15291880 AT A); (20397 15292867 G A); (20398 15293994 G A); (20399 15294152 T C); (20400
15295633 T C); (20401 15296328 C A); (20402 15298296 G A); (20403 15298858 A G); (20404 15299041
A C); (20405 15299349 AT A); (20406 15299656 T C); (20407 15300897 T G); (20408 15301305 C
T); (20409 15301583 G A); (20410 15301595 C T); (20411 15301633 C T); (20412 15302283 A G); (20413
15302633 C T); (20414 15302834 G A); (20415 15303946 G A); (20416 15304074 G A); (20417 15304262
T C); (20418 15306230 C T); (20419 15306266 C T); (20420 15306386 T C); (20421 15306931 A T); (20422
15307548 A G); (20423 15307610 G A); (20424 15307661 C G); (20425 15307915 T A); (20426 15307989
G C); (20427 15308097 G A); (20428 15308669 C T); (20429 15309260 C T); (20430 15309470 G
A); (20431 15309607 G A); (20432 15310040 C T); (20433 15310059 G A); (20434 15310576 T C); (20435
15311328 G A); (20436 15311621 A G); (20437 15312100 G A); (20438 15312493 C T); (20439 15312608
C T); (20440 15312696 T C); (20441 15312725 G T); (20442 15312898 C T); (20443 15312901 A G); (20444
15313360 G C); (20445 15314115 T C); (20446 15314226 C T); (20447 15314398 C T); (20448 15314757 A
G); (20449 15315250 T G); (20450 15315371 ATAAATACAAGGCC A); (20451 15315455 T C); (20452
15315501 C T); (20453 15316128 C A); (20454 15316616 T G); (20455 15317016 G A); (20456 15317204
G A); (20457 15317348 G A); (20458 15317567 C T); (20459 15317609 G A); (20460 15317646 G
T); (20461 15317648 T C); (20462 15318066 T C); (20463 15318110 G A); (20464 15318266 A G); (20465
15318337 C T); (20466 15318421 T C); (20467 15319030 C T); (20468 15319060 T C); (20469 15319350 T
C); (20470 15319489 T C); (20471 15319603 A G); (20472 15319615 G A); (20473 15319623 T C); (20474
15320825 G A); (20475 15321307 C T); (20476 15321963 G A); (20477 15322206 G A); (20478 15322272
G A); (20479 15322935 G T); (20480 15322970 A T); (20481 15324413 A C); (20482 15324621 TA
T); (20483 15325007 T G); (20484 15325513 G A); (20485 15326068 G A); (20486 15328211 C T); (20487
15328408 A G); (20488 15328448 G T); (20489 15328573 T C); (20490 15332271 A G); (20491 15335054
A C); (20492 15335295 A G); (20493 15335328 G A); (20494 15336162 T C); (20495 15336219 T
C); (20496 15336348 G A); (20497 15337063 G T); (20498 15337437 G A); (20499 15337467 C T); (20500
15337486 C T); (20501 15337541 A G); (20502 15337841 A C); (20503 15338051 G A); (20504 15340044
G A); (20505 15340365 G T); (20506 15341236 G A); (20507 15341282 C T); (20508 15341923 A
C); (20509 15341924 A G); (20510 15342289 A G); (20511 15342563 C T); (20512 15342764 C T); (20513
15342937 C A); (20514 15343305 C G); (20515 15343344 G C); (20516 15343635 G A); (20517 15343793
G C); (20518 15343890 G A); (20519 15343994 G A); (20520 15344022 C T); (20521 15344032 G
A); (20522 15344154 T C); (20523 15345103 C T); (20524 15345517 A G); (20525 15345637 G A); (20526
15345641 C T); (20527 15345749 G A); (20528 15345751 T C); (20529 15345876 C T); (20530 15346058
G A); (20531 15346406 C A); (20532 15346589 A G); (20533 15347257 C T); (20534 15347593 C
T); (20535 15348643 A G); (20536 15348713 A G); (20537 15349058 A G); (20538 15349157 G T); (20539
15349465 A C); (20540 15349796 G T); (20541 15350207 A C); (20542 15351927 T A); (20543 15352385
A G); (20544 15352471 G A); (20545 15352885 A G); (20546 15353490 T A); (20547 15353882 T
C); (20548 15354018 C A); (20549 15354038 C A); (20550 15354141 G A); (20551 15354355 T A); (20552
15354786 C CT); (20553 15355173 T C); (20554 15355194 G A); (20555 15355702 G A); (20556 15356116
C T); (20557 15356198 T A); (20558 15356678 C A); (20559 15356961 C A); (20560 15358377 C T); (20561
15358868 T C); (20562 15359370 G A); (20563 15359521 C T); (20564 15360639 C CT); (20565 15360668
T G); (20566 15360717 T C); (20567 15360926 A AT); (20568 15361039 G A); (20569 15361791 T
G); (20570 15361928 A G); (20571 15361994 A G); (20572 15362208 C T); (20573 15362444 A G); (20574
15362570 C T); (20575 15362632 A C); (20576 15363143 A C); (20577 15363148 C A); (20578 15363704
T C); (20579 15364163 A G); (20580 15364395 G A); (20581 15364804 T C); (20582 15368742 C
T); (20583 15369113 G A); (20584 15375069 G A); (20585 15375210 C G); (20586 15375544 C T); (20587
15376245 C T); (20588 15376305 T C); (20589 15376490 G A); (20590 15376803 A G); (20591 15377731
A C); (20592 15378113 T A); (20593 15378334 C T); (20594 15379075 A G); (20595 15379392 C
A); (20596 15379453 G T); (20597 15379454 T C); (20598 15379589 G A); (20599 15379930 G A); (20600
15379953 C T); (20601 15380014 A G); (20602 15380125 G T); (20603 15380645 C T); (20604 15381046
G A); (20605 15381072 G A); (20606 15381645 C T); (20607 15381683 C T); (20608 15381698 T
C); (20609 15381997 G A); (20610 15382004 C T); (20611 15382305 CT C); (20612 15382463 A G); (20613
15382537 T C); (20614 15382979 C T); (20615 15383017 C T); (20616 15383041 C T); (20617 15383527 T
C); (20618 15384278 A G); (20619 15384615 A T); (20620 15384700 T C); (20621 15384728 AC A); (20622
15384926 C T); (20623 15385596 G A); (20624 15386066 T C); (20625 15386666 T TA); (20626 15387032
A T); (20627 15387064 G C); (20628 15387138 T C); (20629 15388195 G T); (20630 15388435 C
G); (20631 15389405 G GAC); (20632 15389985 G A); (20633 15390136 A G); (20634 15390335 C
T); (20635 15390861 G T); (20636 15390884 C G); (20637 15392067 C T); (20638 15393146 C A); (20639
15393463 G A); (20640 15394632 G A); (20641 15394911 C T); (20642 15395702 C T); (20643 15397003
A G); (20644 15397108 C T); (20645 15397380 C T); (20646 15398214 A G); (20647 15398243 C
T); (20648 15398410 G T); (20649 15398609 A G); (20650 15398623 C A); (20651 15398877 G A); (20652
15399391 C T); (20653 15400359 C T); (20654 15400369 C T); (20655 15400590 C T); (20656 15400635 T
C); (20657 15401425 C T); (20658 15401515 T C); (20659 15401667 A G); (20660 15401914 T C); (20661
15401964 C T); (20662 15402066 C T); (20663 15402227 G C); (20664 15403104 C T); (20665 15403407 T
C); (20666 15403453 A G); (20667 15403485 A G); (20668 15403500 C T); (20669 15404406 G C); (20670
15404710 C T); (20671 15405155 G A); (20672 15406982 G T); (20673 15407069 C T); (20674 15407219
G A); (20675 15407220 A G); (20676 15407304 A ATG); (20677 15409179 C T); (20678 15409186 T
C); (20679 15409743 G A); (20680 15409774 A G); (20681 15411386 A C); (20682 15411746 G A); (20683
15411801 A G); (20684 15411924 C T); (20685 15411928 G A); (20686 15411994 G A); (20687 15412824
T C); (20688 15413467 C T); (20689 15415804 C T); (20690 15416166 A T); (20691 15417211 T A); (20692
15418075 G A); (20693 15419032 T G); (20694 15420338 T A); (20695 15420831 A G); (20696 15420954
A C); (20697 15421484 G A); (20698 15421762 A G); (20699 15423294 G C); (20700 15424109 C
T); (20701 15424393 G A); (20702 15424749 G A); (20703 15426160 C T); (20704 15426180 G T); (20705
15426186 C T); (20706 15426436 T C); (20707 15426797 A G); (20708 15426897 C A); (20709 15426969
C T); (20710 15427061 T C); (20711 15427189 A G); (20712 15427483 G A); (20713 15427484 A
T); (20714 15428319 T C); (20715 15428548 C A); (20716 15430558 C T); (20717 15430697 T C); (20718
15431360 A G); (20719 15431762 A T); (20720 15432483 G A); (20721 15433994 G A); (20722 15433996
T C); (20723 15434295 C T); (20724 15434399 C T); (20725 15435005 T C); (20726 15435412 G A); (20727
15435825 A T); (20728 15435948 A T); (20729 15438885 A AGAAACT); (20730 15439263 C T); (20731
15440289 GT G); (20732 15440347 G GAAAAATCGTCATCGTATGTT); (20733 15440515 A G); (20734
15440664 C CTA); (20735 15440703 C A); (20736 15441665 G A); (20737 15442413 C T); (20738
15443525 A G); (20739 15444121 C T); (20740 15444123 G T); (20741 15444135 T A); (20742 15444152
G T); (20743 15444542 C T); (20744 15444636 T C); (20745 15444781 C T); (20746 15444866 C T); (20747
15444916 C T); (20748 15444944 T C); (20749 15445034 G T); (20750 15445070 C G); (20751 15445310
A G); (20752 15445634 G A); (20753 15445943 G A); (20754 15446296 T A); (20755 15446343 C
T); (20756 15446348 T C); (20757 15447229 C A); (20758 15447473 C T); (20759 15447521 C T); (20760
15447612 G A); (20761 15447885 C T); (20762 15447936 C T); (20763 15448025 T C); (20764 15448170 C
A); (20765 15448792 A G); (20766 15449005 C T); (20767 15449098 A C); (20768 15449304 G A); (20769
15450019 G A); (20770 15450044 G A); (20771 15450177 A G); (20772 15450218 TCTTTA T); (20773
15450900 A G); (20774 15451613 A G); (20775 15452742 G A); (20776 15452804 A G); (20777 15452806
C T); (20778 15453241 T C); (20779 15453243 G T); (20780 15453247 A G); (20781 15453374 A
AT); (20782 15454242 G A); (20783 15454631 A C); (20784 15454639 T C); (20785 15455346 T C); (20786
15456472 G A); (20787 15456526 A G); (20788 15457342 T C); (20789 15457436 GTT G); (20790
15457647 G A); (20791 15457856 C A); (20792 15458408 A G); (20793 15458689 T C); (20794 15458929
A G); (20795 15459579 T A); (20796 15459727 C T); (20797 15459860 C A); (20798 15460320 C T); (20799
15460343 C T); (20800 15460672 G A); (20801 15464084 GT G); (20802 15464143 T C); (20803 15464163
C T); (20804 15464164 A G); (20805 15464165 G A); (20806 15464166 A G); (20807 15472856 T
C); (20808 15474384 C T); (20809 15474562 A C); (20810 15474794 C A); (20811 15476789 C A); (20812
15477201 A C); (20813 15477468 C T); (20814 15477913 C T); (20815 15478001 A C); (20816 15479091
G T); (20817 15479123 G A); (20818 15479799 A T); (20819 15479826 A G); (20820 15481340 C
T); (20821 15481518 A C); (20822 15481887 T C); (20823 15482034 A G); (20824 15482263 A G); (20825
15482374 C T); (20826 15482463 AT A); (20827 15483236 G A); (20828 15484288 C T); (20829 15484947
G A); (20830 15486636 T C); (20831 15486999 C T); (20832 15487193 A G); (20833 15488130 A
G); (20834 15488250 C T); (20835 15488278 G A); (20836 15488860 A T); (20837 15489069 T C); (20838
15489865 C T); (20839 15489895 T C); (20840 15490236 A G); (20841 15491156 C A); (20842 15491523
A G); (20843 15491973 C T); (20844 15492739 TA T); (20845 15493263 C T); (20846 15493303 G
A); (20847 15493491 A G); (20848 15493904 T G); (20849 15494078 G A); (20850 15494194 C T); (20851
15494262 G A); (20852 15494454 T G); (20853 15494919 G A); (20854 15495014 A G); (20855 15496109
G A); (20856 15496147 C T); (20857 15496282 A G); (20858 15496389 A G); (20859 15496875 C
A); (20860 15497904 T C); (20861 15498338 C CTA); (20862 15498609 G T); (20863 15498655 G
A); (20864 15499768 T C); (20865 15499950 C T); (20866 15500125 T C); (20867 15500462 A G); (20868
15501315 G A); (20869 15501749 A G); (20870 15503477 G A); (20871 15503610 G A); (20872 15505796
TCAA T); (20873 15506537 C T); (20874 15506553 T C); (20875 15506941 A C); (20876 15508615 C
T); (20877 15509441 T C); (20878 15509635 C T); (20879 15509642 G A); (20880 15509731 C T); (20881
15510027 C G); (20882 15510323 G A); (20883 15510563 G A); (20884 15512757 T C); (20885 15512830
A G); (20886 15512989 A G); (20887 15512999 C A); (20888 15513708 A G); (20889 15513720 A
T); (20890 15513986 G T); (20891 15514168 G A); (20892 15514339 T G); (20893 15514411 C G); (20894
15514466 A G); (20895 15515102 G A); (20896 15515159 A C); (20897 15515677 T C); (20898 15516248
A G); (20899 15517034 C T); (20900 15517665 C A); (20901 15517762 T A); (20902 15517799 G
A); (20903 15517971 A T); (20904 15518482 A C); (20905 15519590 T C); (20906 15519966 A T); (20907
15520324 T G); (20908 15520649 A T); (20909 15520700 G A); (20910 15520891 T A); (20911 15521499
T C); (20912 15521681 G A); (20913 15521723 T C); (20914 15521874 C T); (20915 15522043 A
G); (20916 15522250 C G); (20917 15522359 TTTTGAAATCGTGCTCAGCTC T); (20918 15522812 G
A); (20919 15522844 T C); (20920 15523307 C T); (20921 15524081 C A); (20922 15524934 C T); (20923
15525077 G A); (20924 15525206 C A); (20925 15525279 A C); (20926 15525509 G A); (20927 15525908
A T); (20928 15526088 G A); (20929 15526654 T C); (20930 15527419 T G); (20931 15527885 C T); (20932
15528135 C T); (20933 15528939 T C); (20934 15528950 G A); (20935 15529425 C T); (20936 15530625 T
TGA); (20937 15530922 TA T); (20938 15531118 T A); (20939 15531262 G A); (20940 15532892 A
T); (20941 15535146 A C); (20942 15541352 C G); (20943 15541463 T G); (20944 15543614 T A); (20945
15545332 T G); (20946 15545479 T G); (20947 15545649 C T); (20948 15545696 G A); (20949 15545888
T C); (20950 15546083 C T); (20951 15546392 C T); (20952 15546600 G A); (20953 15548783 G
A); (20954 15549306 G A); (20955 15549320 T G); (20956 15549485 C T); (20957 15550619 G A); (20958
15551385 A G); (20959 15551720 G A); (20960 15552945 G A); (20961 15553858 G T); (20962 15553944
G T); (20963 15554007 G A); (20964 15554013 A G); (20965 15554068 G A); (20966 15554115 G
A); (20967 15555242 G A); (20968 15555348 C T); (20969 15555455 C T); (20970 15555506 A G); (20971
15555683 A C); (20972 15555853 A G); (20973 15555925 C T); (20974 15556559 G A); (20975 15557073
A T); (20976 15557535 T C); (20977 15558114 G A); (20978 15558272 C A); (20979 15558285 C T); (20980
15558303 A G); (20981 15558335 T G); (20982 15558382 T C); (20983 15558426 G A); (20984 15559283
A C); (20985 15559792 A G); (20986 15560978 T C); (20987 15561140 A C); (20988 15562918 G
A); (20989 15563476 G A); (20990 15563486 G A); (20991 15563835 C A); (20992 15566196 C T); (20993
15566681 G A); (20994 15567280 A G); (20995 15567437 A G); (20996 15567461 G A); (20997 15567699
T G); (20998 15568213 A G); (20999 15568672 T C); (21000 15568840 T C); (21001 15569031 T A); (21002
15569311 A G); (21003 15569392 A AC); (21004 15569461 T C); (21005 15569720 G A); (21006
15570597 A G); (21007 15570710 G A); (21008 15570758 C T); (21009 15571896 G A); (21010 15572759
C A); (21011 15572824 G A); (21012 15572825 A G); (21013 15573730 A T); (21014 15574083 T
C); (21015 15574188 G A); (21016 15574485 T C); (21017 15574968 A G); (21018 15575215 T C); (21019
15575608 A G); (21020 15575896 C A); (21021 15576131 T C); (21022 15576208 A T); (21023 15576397
A G); (21024 15576593 G T); (21025 15576820 A G); (21026 15577232 C T); (21027 15577724 G
A); (21028 15577794 T C); (21029 15577835 A G); (21030 15578026 T A); (21031 15578639 C A); (21032
15579469 C T); (21033 15582081 C T); (21034 15582089 A G); (21035 15582365 C T); (21036 15583136 C
T); (21037 15583312 TAGAG T); (21038 15583736 C T); (21039 15583992 T C); (21040 15583993 T
G); (21041 15584555 C G); (21042 15584767 C T); (21043 15584886 G A); (21044 15584889 A G); (21045
15585088 T C); (21046 15585852 T C); (21047 15586206 C T); (21048 15586708 C T); (21049 15586799 A
G); (21050 15586869 G T); (21051 15587183 T C); (21052 15587240 C T); (21053 15587280 T G); (21054
15587624 A T); (21055 15587812 C T); (21056 15588259 G T); (21057 15588550 G A); (21058 15588562
T C); (21059 15589117 T C); (21060 15589614 T G); (21061 15589974 G T); (21062 15590695 T C); (21063
15591182 C T); (21064 15591891 G T); (21065 15591927 A G); (21066 15592019 T C); (21067 15592169
A T); (21068 15592281 T C); (21069 15592527 G A); (21070 15592680 G A); (21071 15592769 C
T); (21072 15594962 T C); (21073 15595597 A G); (21074 15596202 A G); (21075 15596322 A G); (21076
15596330 T G); (21077 15596375 G A); (21078 15597840 C A); (21079 15598464 T G); (21080 15598511
G T); (21081 15598512 A T); (21082 15598551 A G); (21083 15598711 G T); (21084 15599352 T
C); (21085 15599410 T C); (21086 15599411 C G); (21087 15599412 A T); (21088 15599413 A C); (21089
15599414 T A); (21090 15599415 G A); (21091 15599416 A T); (21092 15599417 C G); (21093 15599615
G T); (21094 15599619 G A); (21095 15599837 T A); (21096 15600490 T C); (21097 15606672 G
A); (21098 15606674 G A); (21099 15606724 G A); (21100 15606866 G A); (21101 15608440 T C); (21102
15608673 A T); (21103 15609105 C T); (21104 15609466 A T); (21105 15609515 A G); (21106 15609917
C G); (21107 15610220 G T); (21108 15610709 GTCCT G); (21109 15611315 A G); (21110 15611912 C
T); (21111 15612348 A G); (21112 15612393 C T); (21113 15614494 C T); (21114 15614720 AT A); (21115
15615337 A AC); (21116 15615385 T G); (21117 15615416 C A); (21118 15615977 C T); (21119 15616231
T C); (21120 15616322 T C); (21121 15616795 C T); (21122 15617870 T C); (21123 15618022 AT
A); (21124 15618111 C T); (21125 15618284 T C); (21126 15618959 A C); (21127 15618966 C T); (21128
15619667 G A); (21129 15620353 C T); (21130 15620918 T G); (21131 15621541 C T); (21132 15621589
G A); (21133 15621690 A C); (21134 15621776 G A); (21135 15621963 T G); (21136 15622001 G
T); (21137 15622142 C T); (21138 15622400 G C); (21139 15623277 C T); (21140 15624439 G A); (21141
15624751 C T); (21142 15625045 G A); (21143 15625168 A G); (21144 15625445 A C); (21145 15625508
C T); (21146 15625681 G A); (21147 15625914 A G); (21148 15627134 T C); (21149 15628286 A
G); (21150 15629032 C A); (21151 15629908 G A); (21152 15631061 G GC); (21153 15632260 C
T); (21154 15632277 T C); (21155 15632280 T A); (21156 15632426 C T); (21157 15632820 G C); (21158
15632894 C T); (21159 15633161 A G); (21160 15633442 A G); (21161 15633916 G A); (21162 15634429
G A); (21163 15634464 T C); (21164 15634788 G A); (21165 15634861 T C); (21166 15635274 G
T); (21167 15636097 G A); (21168 15636293 G A); (21169 15636304 G A); (21170 15638210 T C); (21171
15638293 G A); (21172 15638678 T G); (21173 15639148 C T); (21174 15639185 T G); (21175 15639411
A C); (21176 15639558 C T); (21177 15639702 C T); (21178 15639945 G A); (21179 15640163 G
C); (21180 15640373 G A); (21181 15641075 G T); (21182 15641248 T C); (21183 15641900 G A); (21184
15642117 A T); (21185 15642273 G A); (21186 15642612 GT G); (21187 15644080 C G); (21188
15644258 T C); (21189 15644293 G A); (21190 15644369 C T); (21191 15645251 C T); (21192 15646093 T
C); (21193 15646656 A G); (21194 15647391 A G); (21195 15648144 G GA); (21196 15648468 T
C); (21197 15649802 A C); (21198 15650998 C T); (21199 15651013 A G); (21200 15651020 G A); (21201
15651062 C T); (21202 15651483 C T); (21203 15651560 T A); (21204 15651632 C T); (21205 15651809 A
G); (21206 15653078 C T); (21207 15653402 G A); (21208 15653511 C A); (21209 15654143 A C); (21210
15654954 T C); (21211 15655441 C T); (21212 15659380 G T); (21213 15659648 A G); (21214 15659839
C T); (21215 15660562 A C); (21216 15660597 A T); (21217 15660758 TG T); (21218 15660980 G
A); (21219 15662691 C T); (21220 15663094 T C); (21221 15663717 G T); (21222 15663935 G A); (21223
15664470 G A); (21224 15664624 G A); (21225 15665570 A G); (21226 15665766 C T); (21227 15665782
A G); (21228 15665994 A G); (21229 15666663 A G); (21230 15668425 G C); (21231 15668783 C
T); (21232 15669326 A G); (21233 15669555 G A); (21234 15669733 G A); (21235 15669769 A T); (21236
15669876 G A); (21237 15669884 G A); (21238 15670014 T C); (21239 15670355 T C); (21240 15670592
T C); (21241 15670650 G A); (21242 15671312 G A); (21243 15671940 C T); (21244 15674454 T
C); (21245 15674458 G A); (21246 15674486 A G); (21247 15674569 C T); (21248 15674687 A G); (21249
15674692 G A); (21250 15676204 C T); (21251 15678277 G C); (21252 15678759 C T); (21253 15679210
A G); (21254 15680133 A C); (21255 15680160 G A); (21256 15682156 G A); (21257 15682403 G
C); (21258 15683411 C T); (21259 15684035 T A); (21260 15684515 T C); (21261 15685182 G A); (21262
15685220 C T); (21263 15685276 A G); (21264 15685281 G A); (21265 15685291 T C); (21266 15685472
A T); (21267 15685488 T C); (21268 15686437 A T); (21269 15686696 A G); (21270 15686839 C
CATGAATGGAATATGATTGTTGGTATGTGAA); (21271 15686888 T A); (21272 15687358 C T); (21273
15687494 T C); (21274 15687981 C T); (21275 15689107 T G); (21276 15689540 A G); (21277 15689681
C T); (21278 15690535 T C); (21279 15690657 C T); (21280 15691095 T C); (21281 15691508 T C); (21282
15691677 T C); (21283 15691836 A G); (21284 15692305 C T); (21285 15692493 G C); (21286 15692647
C T); (21287 15693036 G A); (21288 15693247 T C); (21289 15693441 A G); (21290 15693542 G
A); (21291 15694482 C T); (21292 15694521 T C); (21293 15694902 T A); (21294 15695472 A G); (21295
15696035 T C); (21296 15696137 C A); (21297 15696838 G T); (21298 15697294 T C); (21299 15698002 C
A); (21300 15698189 T G); (21301 15698402 A T); (21302 15698988 T A); (21303 15699110 G T); (21304
15699324 C T); (21305 15699414 T C); (21306 15699480 C T); (21307 15699625 C G); (21308 15699761 C
T); (21309 15699925 C A); (21310 15701458 T C); (21311 15701610 G A); (21312 15702566 C T); (21313
15702583 A G); (21314 15702932 A G); (21315 15702959 A C); (21316 15703205 A AT); (21317
15703350 C A); (21318 15703396 C T); (21319 15703878 C A); (21320 15704135 G A); (21321 15705029
T G); (21322 15705305 C T); (21323 15706578 C T); (21324 15706980 C T); (21325 15707068 T C); (21326
15707368 A C); (21327 15707630 G A); (21328 15708449 C T); (21329 15709438 C T); (21330 15709778
C A); (21331 15710601 C T); (21332 15710729 A G); (21333 15710793 C T); (21334 15710969 G
A); (21335 15711580 T C); (21336 15712138 G A); (21337 15712549 G A); (21338 15712581 A G); (21339
15712797 A G); (21340 15712998 A T); (21341 15713517 T C); (21342 15713934 A C); (21343 15714785
A G); (21344 15715244 G A); (21345 15715429 T C); (21346 15716446 G A); (21347 15717382 G
A); (21348 15717476 G C); (21349 15717528 G C); (21350 15718836 A C); (21351 15718841 T C); (21352
15719367 TG T); (21353 15719657 C A); (21354 15719667 C T); (21355 15720169 T C); (21356 15720234
G T); (21357 15721282 G A); (21358 15722344 T C); (21359 15723229 C CACTAA); (21360 15723383 A
G); (21361 15723394 G C); (21362 15723420 C T); (21363 15723843 G A); (21364 15724241 T C); (21365
15724321 A G); (21366 15724593 G A); (21367 15725028 A T); (21368 15725819 A G); (21369 15725885
G A); (21370 15725933 C T); (21371 15726573 C T); (21372 15727923 C A); (21373 15727925 C T); (21374
15728959 A G); (21375 15729148 A C); (21376 15730615 G T); (21377 15730756 C T); (21378 15731497
C A); (21379 15731596 G A); (21380 15731722 C T); (21381 15732138 T C); (21382 15732155 T C); (21383
15733261 T A); (21384 15733707 T C); (21385 15733869 C T); (21386 15733908 T G); (21387 15734590 T
C); (21388 15734697 C T); (21389 15735032 A G); (21390 15736018 C T); (21391 15737138 G A); (21392
15739415 G A); (21393 15739550 T C); (21394 15740076 C A); (21395 15740540 T C); (21396 15740542
T C); (21397 15740856 T C); (21398 15740865 T C); (21399 15741056 C T); (21400 15741222 C
CAG); (21401 15741793 C T); (21402 15741965 T C); (21403 15741966 C T); (21404 15741967 A
T); (21405 15742948 T C); (21406 15743338 C T); (21407 15743598 G A); (21408 15744916 T C); (21409
15744994 G T); (21410 15745939 C A); (21411 15746905 G A); (21412 15747445 C A); (21413 15748236
CAAATATTTTTGAAAACGCACCTTATCGGT C); (21414 15750621 A G); (21415 15751412 G A); (21416
15751859 G A); (21417 15752196 C A); (21418 15753443 G A); (21419 15753450 C T); (21420 15753844
C T); (21421 15754289 G C); (21422 15754538 G T); (21423 15754577 A C); (21424 15756083 C T); (21425
15756220 C T); (21426 15756255 T G); (21427 15757219 C T); (21428 15758037 G A); (21429 15758490
C T); (21430 15758492 T C); (21431 15758498 C T); (21432 15758500 T C); (21433 15758512 C T); (21434
15758514 T TATAC); (21435 15758538 TATATATATATATAC T); (21436 15759024 G A); (21437 15759030
G A); (21438 15759757 G GT); (21439 15760026 C CT); (21440 15760047 C T); (21441 15760054 C
T); (21442 15760389 T G); (21443 15760857 GA G); (21444 15761624 A G); (21445 15761885 T
A); (21446 15762298 T C); (21447 15762676 C T); (21448 15765370 G A); (21449 15765512 A AT); (21450
15765645 A G); (21451 15765670 G A); (21452 15765953 G T); (21453 15766152 A G); (21454 15766241
C T); (21455 15766331 T A); (21456 15766582 C T); (21457 15766965 A G); (21458 15767011 C
G); (21459 15767028 C T); (21460 15767243 T C); (21461 15767255 A G); (21462 15767683 A G); (21463
15767707 G A); (21464 15767882 A G); (21465 15768023 G A); (21466 15768031 C T); (21467 15768300
C T); (21468 15768391 T C); (21469 15769003 C T); (21470 15769044 T G); (21471 15769070 A G); (21472
15769671 G A); (21473 15769725 G A); (21474 15770660 C T); (21475 15772627 G A); (21476 15772731
C CT); (21477 15773232 G A); (21478 15774289 T A); (21479 15774292 T A); (21480 15774293 A
T); (21481 15774402 TA T); (21482 15776148 A T); (21483 15777107 T G); (21484 15777545 A C); (21485
15777720 C T); (21486 15778244 C T); (21487 15779250 G T); (21488 15780551 G A); (21489 15781031
G A); (21490 15781686 G C); (21491 15782057 G T); (21492 15782412 A C); (21493 15782609 C
T); (21494 15783313 T C); (21495 15783809 TAGTGTACACATTTG T); (21496 15786133 T C); (21497
15787069 A G); (21498 15789217 T A); (21499 15789603 T C); (21500 15789642 TG T); (21501 15791134
T C); (21502 15791910 C CATAT); (21503 15792034 C T); (21504 15792067 T C); (21505 15792478 A
G); (21506 15794246 T C); (21507 15794486 TTA T); (21508 15794987 C A); (21509 15795078 A
G); (21510 15796406 C T); (21511 15796682 C G); (21512 15797110 G T); (21513 15798628 C T); (21514
15799250 G A); (21515 15800330 G T); (21516 15800461 T C); (21517 15800916 T C); (21518 15801487
G GTGTTGAA); (21519 15802775 T G); (21520 15803466 T C); (21521 15807254 T G); (21522 15808019 C
T); (21523 15808781 G A); (21524 15809968 T A); (21525 15810082 C T); (21526 15810211 A G); (21527
15810578 C T); (21528 15812556 G T); (21529 15812649 G T); (21530 15815885 A T); (21531 15816254
G A); (21532 15816604 C G); (21533 15817050 G A); (21534 15817873 T C); (21535 15818028 G
A); (21536 15818153 G T); (21537 15818708 T C); (21538 15818730 G A); (21539 15818802 C T); (21540
15818906 T G); (21541 15819006 G C); (21542 15819820 A G); (21543 15821936 C T); (21544 15822209
A G); (21545 15822952 T C); (21546 15824732 A G); (21547 15825849 C CA); (21548 15825999 G
T); (21549 15826187 C T); (21550 15827444 C A); (21551 15827541 G A); (21552 15827635 T C); (21553
15828092 C G); (21554 15829028 G A); (21555 15829271 T G); (21556 15829830 G GACA); (21557
15830413 G T); (21558 15830845 G C); (21559 15832090 T TA); (21560 15833535 G A); (21561
15833696 C T); (21562 15833874 G A); (21563 15833933 G A); (21564 15834361 A G); (21565 15834380
A G); (21566 15834530 G A); (21567 15836004 C T); (21568 15836016 T C); (21569 15836052 G
A); (21570 15838736 A G); (21571 15839019 G A); (21572 15839363 G A); (21573 15839659 C T); (21574
15839834 G T); (21575 15840633 A G); (21576 15841214 G T); (21577 15841656 T C); (21578 15842195
G T); (21579 15842524 A G); (21580 15842628 G C); (21581 15844633 G A); (21582 15844862 A
G); (21583 15845160 T G); (21584 15845646 T G); (21585 15846037 C T); (21586 15847534 A G); (21587
15847674 T A); (21588 15847712 C G); (21589 15847746 G A); (21590 15847969 G
GTCTATCATAGTGT); (21591 15848067 C T); (21592 15848561 A G); (21593 15848606 A G); (21594
15848634 C T); (21595 15850073 G T); (21596 15850162 T C); (21597 15850636 C T); (21598 15850902 A
C); (21599 15851018 A G); (21600 15851049 G A); (21601 15852448 T C); (21602 15852774 A G); (21603
15852858 A C); (21604 15853009 C T); (21605 15853260 C G); (21606 15853857 A G); (21607 15854176
C CT); (21608 15854454 G A); (21609 15855799 G A); (21610 15856678 T A); (21611 15857029 T
C); (21612 15857287 G A); (21613 15857924 G T); (21614 15858259 G A); (21615 15858538 T C); (21616
15858622 C T); (21617 15858726 G A); (21618 15858884 C T); (21619 15859191 C T); (21620 15860035 T
C); (21621 15862963 G T); (21622 15863375 T C); (21623 15864175 A AT); (21624 15864272 A C); (21625
15864407 T C); (21626 15864420 G A); (21627 15864536 A G); (21628 15864721 G A); (21629 15864899
G A); (21630 15864901 C T); (21631 15865291 T C); (21632 15865662 AT A); (21633 15866933 C
A); (21634 15867549 C T); (21635 15867923 T C); (21636 15869193 C T); (21637 15869324 T C); (21638
15869868 C T); (21639 15870476 C T); (21640 15870946 A G); (21641 15871825 C T); (21642 15872076
G T); (21643 15872629 A G); (21644 15874208 G C); (21645 15874282 C A); (21646 15874283 C
T); (21647 15874402 G C); (21648 15875060 T C); (21649 15875217 C T); (21650 15876328 A G); (21651
15876768 T C); (21652 15877098 C T); (21653 15878293 C T); (21654 15879198 A G); (21655 15879566
G A); (21656 15879604 A G); (21657 15879737 G A); (21658 15880094 G A); (21659 15880295 A
G); (21660 15880465 G A); (21661 15881139 T C); (21662 15881426 G A); (21663 15881463 T C); (21664
15881917 T C); (21665 15882236 T C); (21666 15882417 C T); (21667 15882563 C A); (21668 15882600 T
G); (21669 15882814 T C); (21670 15883199 A G); (21671 15883240 G A); (21672 15883648 G A); (21673
15884906 G T); (21674 15885168 T C); (21675 15885276 C A); (21676 15885403 C T); (21677 15885404 T
A); (21678 15885405 A G); (21679 15885406 A G); (21680 15885420 GT G); (21681 15886724 A
G); (21682 15887069 C G); (21683 15887182 T G); (21684 15887353 A C); (21685 15887570 A T); (21686
15888021 A C); (21687 15889295 T A); (21688 15890860 T G); (21689 15890891 A AAT); (21690
15893078 G T); (21691 15893838 C T); (21692 15894013 C T); (21693 15895115 T C); (21694 15895130
TC T); (21695 15895180 A G); (21696 15895673 G A); (21697 15895709 A G); (21698 15895921 C
T); (21699 15896423 G T); (21700 15897075 GT G); (21701 15897346 C T); (21702 15897626 G T); (21703
15898417 A T); (21704 15899033 T C); (21705 15899821 G A); (21706 15902152 A G); (21707 15902612
A G); (21708 15902909 A C); (21709 15903405 G C); (21710 15904429 T A); (21711 15904682 G
A); (21712 15905575 T C); (21713 15905659 C T); (21714 15905689 T TG); (21715 15905691 G T); (21716
15906238 T G); (21717 15907813 C T); (21718 15907907 A G); (21719 15907967 T C); (21720 15907989
T A); (21721 15909165 G A); (21722 15909185 T C); (21723 15909529 C T); (21724 15911137 G
A); (21725 15912061 G T); (21726 15912189 C G); (21727 15913382 T C); (21728 15913919 C T); (21729
15914522 A G); (21730 15915292 T A); (21731 15916155 C T); (21732 15916650 C A); (21733 15917275
CT C); (21734 15917586 A AT); (21735 15917833 A G); (21736 15918237 T C); (21737 15918629 T
C); (21738 15918662 T C); (21739 15918746 G A); (21740 15918928 G T); (21741 15921256 C T); (21742
15921570 G GTT); (21743 15922683 C G); (21744 15923167 C T); (21745 15923669 G GA); (21746
15923937 G A); (21747 15924018 C T); (21748 15924131 T C); (21749 15925387 G A); (21750 15927608
A T); (21751 15927815 T G); (21752 15930731 C G); (21753 15930762 C A); (21754 15932133 A
T); (21755 15932788 A G); (21756 15932865 C T); (21757 15933114 C T); (21758 15933186 C T); (21759
15933578 C T); (21760 15933801 A C); (21761 15934231 A G); (21762 15934232 C T); (21763 15934298
A T); (21764 15936015 C T); (21765 15938015 T G); (21766 15938543 C A); (21767 15938852 A T); (21768
15938854 G GAT); (21769 15939085 T C); (21770 15940156 T C); (21771 15940648 C T); (21772
15941894 A T); (21773 15942049 T C); (21774 15942830 A C); (21775 15943849 C T); (21776 15943861 T
C); (21777 15944662 G T); (21778 15945567 C T); (21779 15945956 G C); (21780 15946397 T C); (21781
15946682 CCTT C); (21782 15947164 G A); (21783 15947803 C T); (21784 15948579 A G); (21785
15949594 G A); (21786 15949882 C T); (21787 15949926 C T); (21788 15949994 C T); (21789 15950106
A G); (21790 15950630 T C); (21791 15950657 G A); (21792 15951198 A C); (21793 15951362 C
T); (21794 15951798 C G); (21795 15952043 G T); (21796 15952269 G C); (21797 15953297 C T); (21798
15954097 G T); (21799 15954805 G C); (21800 15955916 A AT); (21801 15956202 A G); (21802
15956969 G A); (21803 15957108 T C); (21804 15957662 G A); (21805 15960188 GTGTATGTATGTA
G); (21806 15960805 G A); (21807 15960944 A G); (21808 15961171 C G); (21809 15961607 A G); (21810
15961664 G A); (21811 15961783 C G); (21812 15962903 A G); (21813 15962994 A AT); (21814
15964364 T TA); (21815 15964719 T C); (21816 15965050 T C); (21817 15965120 G A); (21818 15966001
T C); (21819 15966479 C T); (21820 15966733 A C); (21821 15969031 G A); (21822 15969850 C T); (21823
15969882 G A); (21824 15970425 T C); (21825 15971269 A T); (21826 15972604 G A); (21827 15972666
T G); (21828 15973574 C T); (21829 15975487 C T); (21830 15975626 T C); (21831 15975633 G A); (21832
15976252 T C); (21833 15977530 A G); (21834 15978585 T TTA); (21835 15979662 C T); (21836
15980047 C T); (21837 15980970 G A); (21838 15981677 T C); (21839 15981678 T A); (21840 15981688
C T); (21841 15981995 A T); (21842 15982256 A T); (21843 15982897 T C); (21844 15983616 G A); (21845
15984721 A C); (21846 15985398 T C); (21847 15985638 G A); (21848 15987094 C T); (21849 15987507
G T); (21850 15987718 G A); (21851 15987728 T G); (21852 15988105 G A); (21853 15988410 G
GA); (21854 15988908 G A); (21855 15988909 C T); (21856 15989075 G A); (21857 15990956 C
A); (21858 15990977 C T); (21859 15991359 C T); (21860 15991384 G A); (21861 15991459 A G); (21862
15992152 T A); (21863 15992205 G A); (21864 15992299 A G); (21865 15992547 A T); (21866 15993090
C T); (21867 15993999 A G); (21868 15994695 C T); (21869 15994753 A G); (21870 15995022 T
G); (21871 15995083 CAA C); (21872 15996588 C T); (21873 15996928 G A); (21874 15997408 A
T); (21875 15997938 A G); (21876 15998666 G A); (21877 16000362 A G); (21878 16000909 T C); (21879
16001029 C T); (21880 16001228 T C); (21881 16001300 G T); (21882 16001460 C T); (21883 16001962 A
G); (21884 16002020 T C); (21885 16002281 G T); (21886 16003038 A G); (21887 16003179 C T); (21888
16003296 A T); (21889 16004597 T C); (21890 16004629 T C); (21891 16005590 C G); (21892 16005902 C
G); (21893 16005930 G A); (21894 16006088 C T); (21895 16006153 G A); (21896 16008723 CAACTT
C); (21897 16009133 T A); (21898 16010076 G A); (21899 16011917 T C); (21900 16012157 G A); (21901
16012244 T G); (21902 16012364 G A); (21903 16012707 T C); (21904 16012914 C T); (21905 16012929
T C); (21906 16012939 G A); (21907 16012998 G A); (21908 16014660 G A); (21909 16014742 T
C); (21910 16014852 T G); (21911 16016108 G T); (21912 16017459 T C); (21913 16017528 G A); (21914
16017712 G A); (21915 16019106 C T); (21916 16019216 C T); (21917 16019301 A G); (21918 16019484
C T); (21919 16019531 C T); (21920 16019826 G A); (21921 16020085 G A); (21922 16020970 G
A); (21923 16021425 G A); (21924 16021478 T C); (21925 16021684 CT C); (21926 16023035 T C); (21927
16023123 T G); (21928 16024316 C T); (21929 16024736 C T); (21930 16025422 A G); (21931 16025547
G GAAAACAAACAAAAAATAT); (21932 16025607 C T); (21933 16026280 C T); (21934 16027431 G
A); (21935 16027451 T A); (21936 16028638 G A); (21937 16029272 C T); (21938 16030985 G A); (21939
16032078 C T); (21940 16032457 G T); (21941 16032483 A T); (21942 16033357 A C); (21943 16034210
GT G); (21944 16034240 G A); (21945 16034585 C T); (21946 16035647 A G); (21947 16037605 C
A); (21948 16037780 G T); (21949 16038977 G C); (21950 16039488 C T); (21951 16039632 T C); (21952
16040123 A C); (21953 16040281 T G); (21954 16040380 T C); (21955 16040421 C T); (21956 16040519
AGTGAT A); (21957 16041985 G A); (21958 16042357 T G); (21959 16042455 G A); (21960 16043601 T
TTTATTATTATTATTATTATTA); (21961 16044467 T A); (21962 16044469 T A); (21963 16045388 C
T); (21964 16046068 A G); (21965 16046183 T A); (21966 16046357 TC T); (21967 16046359 T A); (21968
16046380 C T); (21969 16046510 C T); (21970 16046637 T C); (21971 16047694 GA G); (21972 16049365
A T); (21973 16051185 T C); (21974 16051342 T G); (21975 16051480 CTACAAAAGGCA C); (21976
16051558 A C); (21977 16052396 G T); (21978 16052870 C T); (21979 16053239 A T); (21980 16054738
C CATAT); (21981 16055005 C T); (21982 16055643 T C); (21983 16055731 G T); (21984 16055947 C
T); (21985 16056481 G A); (21986 16057073 A G); (21987 16058039 C A); (21988 16058907 G A); (21989
16059241 T C); (21990 16059255 A G); (21991 16060956 T A); (21992 16060974 C T); (21993 16062120
C T); (21994 16062415 T G); (21995 16063641 C T); (21996 16063724 G A); (21997 16066901 A
G); (21998 16067001 G A); (21999 16068344 C T); (22000 16068524 T C); (22001 16069218 C A); (22002
16069272 C G); (22003 16069492 G A); (22004 16070014 A G); (22005 16070030 C T); (22006 16070121
G A); (22007 16070139 C T); (22008 16070270 G A); (22009 16070885 A G); (22010 16071179 A
G); (22011 16071483 CCGGGGGAGTGGTG C); (22012 16071506 C G); (22013 16072454 C T); (22014
16072523 C T); (22015 16072714 A G); (22016 16072730 G A); (22017 16072779 C T); (22018 16072824
T TTGGCGCTGA); (22019 16073143 G A); (22020 16073244 T TA); (22021 16073999 G A); (22022
16074089 G A); (22023 16074397 C T); (22024 16074578 G A); (22025 16075154 A
ATGGCGCCATCTGGCGCTAC); (22026 16075971 C T); (22027 16077050 C G); (22028 16077213 C
A); (22029 16077775 G C); (22030 16078239 C T); (22031 16079023 A G); (22032 16079942 T G); (22033
16080725 G T); (22034 16081065 A G); (22035 16081183 C A); (22036 16082041 T C); (22037 16083576
T C); (22038 16083707 GT G); (22039 16083866 T A); (22040 16084024 A G); (22041 16084113 A
G); (22042 16088614 C T); (22043 16088677 C A); (22044 16089073 A
AAGTGAAGGTACTTTATACCAAATT); (22045 16089426 T C); (22046 16091363 T A); (22047 16093233 G
A); (22048 16093262 A G); (22049 16093330 C G); (22050 16093391 A G); (22051 16093407 A G); (22052
16093617 G GA); (22053 16095669 T C); (22054 16097972 T C); (22055 16098007 C T); (22056 16098760
T C); (22057 16100690 A C); (22058 16101880 C T); (22059 16102139 G A); (22060 16102389 A
C); (22061 16102767 C G); (22062 16102816 T C); (22063 16103954 T G); (22064 16104240 A G); (22065
16104617 T C); (22066 16107011 T C); (22067 16108223 T C); (22068 16108524 G A); (22069 16108534
A G); (22070 16111125 G C); (22071 16112400 C G); (22072 16112739 CATCCA C); (22073 16112834 C
T); (22074 16113037 G A); (22075 16114202 A G); (22076 16114442 T C); (22077 16114792 C A); (22078
16114891 C T); (22079 16115202 A G); (22080 16115390 G A); (22081 16115526 T C); (22082 16115842
G A); (22083 16115970 A G); (22084 16117844 C T); (22085 16118069 G C); (22086 16118094 C
A); (22087 16118182 C T); (22088 16119139 C T); (22089 16119413 A G); (22090 16119470 C T); (22091
16119581 C T); (22092 16119639 G A); (22093 16119665 T C); (22094 16119693 T C); (22095 16119764 T
C); (22096 16119780 C T); (22097 16119978 C T); (22098 16120127 A G); (22099 16120139 C G); (22100
16120166 C T); (22101 16120432 C T); (22102 16120540 T C); (22103 16120623 A G); (22104 16120775 T
C); (22105 16121412 A G); (22106 16122909 C T); (22107 16124356 A G); (22108 16125117 A G); (22109
16125327 G T); (22110 16125360 T C); (22111 16125575 C T); (22112 16126086 A C); (22113 16126159 T
C); (22114 16127938 G A); (22115 16128036 G A); (22116 16128403 GA G); (22117 16129391 C
A); (22118 16131071 A C); (22119 16131271 AT A); (22120 16131599 T G); (22121 16132143 G A); (22122
16132899 A T); (22123 16132989 T C); (22124 16133111 A C); (22125 16133265 G T); (22126 16133443
A T); (22127 16133489 T A); (22128 16133565 C A); (22129 16134400 G T); (22130 16135346 T C); (22131
16135991 A C); (22132 16136237 C G); (22133 16136673 G A); (22134 16136925 C T); (22135 16137484
C T); (22136 16137816 G C); (22137 16138043 G A); (22138 16138238 C T); (22139 16138399 C T); (22140
16138508 G A); (22141 16139700 A G); (22142 16140493 C T); (22143 16141038 C T); (22144 16141636
T C); (22145 16141641 G A); (22146 16141881 G T); (22147 16142393 A AT); (22148 16143094 C
T); (22149 16144021 G A); (22150 16144158 A G); (22151 16144662 A G); (22152 16145267 T G); (22153
16145734 G A); (22154 16145804 C A); (22155 16145979 C T); (22156 16146657 A G); (22157 16146661
G A); (22158 16146731 G A); (22159 16146854 G A); (22160 16146874 G T); (22161 16147154 A
G); (22162 16147163 G T); (22163 16147210 G C); (22164 16147270 A G); (22165 16147551 A
AT); (22166 16147841 T A); (22167 16148175 A G); (22168 16148286 A T); (22169 16148497 G A); (22170
16148993 C T); (22171 16149678 G T); (22172 16149826 G A); (22173 16149847 T A); (22174 16150318
G A); (22175 16150475 A T); (22176 16151091 C T); (22177 16151092 A C); (22178 16151328 T C); (22179
16151826 C A); (22180 16152492 A C); (22181 16152602 G A); (22182 16153217 G A); (22183 16153874
G A); (22184 16154045 A G); (22185 16154209 C T); (22186 16154366 C T); (22187 16155127 G
T); (22188 16155385 C T); (22189 16155682 C T); (22190 16156538 T C); (22191 16156545 G A); (22192
16156885 T C); (22193 16157204 G A); (22194 16157281 A G); (22195 16158115 A G); (22196 16158129
C T); (22197 16158201 A G); (22198 16158274 G A); (22199 16158425 G A); (22200 16160108 G
A); (22201 16160157 C T); (22202 16160286 G A); (22203 16160881 A T); (22204 16160992 C T); (22205
16161441 A G); (22206 16161661 G A); (22207 16162155 G A); (22208 16162161 G A); (22209 16162802
G T); (22210 16163239 G T); (22211 16163889 C T); (22212 16165661 T C); (22213 16165895 T C); (22214
16166780 A T); (22215 16167025 A G); (22216 16167478 T C); (22217 16168160 G A); (22218 16168942
G A); (22219 16169039 C T); (22220 16169948 T A); (22221 16171249 C T); (22222 16171381 C T); (22223
16172000 A G); (22224 16172121 C T); (22225 16172719 T G); (22226 16172934 C T); (22227 16173135
G A); (22228 16174046 G T); (22229 16174320 C T); (22230 16174359 G C); (22231 16175257 C
T); (22232 16175454 T C); (22233 16175549 T A); (22234 16177568 A G); (22235 16177638 G A); (22236
16177665 G A); (22237 16177989 G A); (22238 16178006 C T); (22239 16178347 A C); (22240 16178461
G A); (22241 16178550 G A); (22242 16178697 T C); (22243 16179046 G A); (22244 16181716 A
G); (22245 16181863 G A); (22246 16184916 A C); (22247 16185152 C T); (22248 16185527 A C); (22249
16185729 T C); (22250 16185770 T G); (22251 16186116 G T); (22252 16186261 G C); (22253 16186315
G A); (22254 16186471 G A); (22255 16186921 G A); (22256 16186971 A G); (22257 16187281 A
T); (22258 16187566 A G); (22259 16187996 G A); (22260 16188571 G A); (22261 16188834 C T); (22262
16189559 A C); (22263 16189560 G A); (22264 16189582 A G); (22265 16189838 G A); (22266 16190512
G A); (22267 16190513 T G); (22268 16190530 T C); (22269 16190892 A G); (22270 16191115 G
A); (22271 16191352 G A); (22272 16192541 T C); (22273 16192724 A T); (22274 16192767 G A); (22275
16193127 A T); (22276 16193167 C G); (22277 16193308 A G); (22278 16193411 G A); (22279 16193474
C CAT); (22280 16193545 C G); (22281 16195463 A G); (22282 16195584 G A); (22283 16196460 T
C); (22284 16197070 G A); (22285 16197465 T G); (22286 16197493 C T); (22287 16198231 G C); (22288
16198680 T C); (22289 16198797 T C); (22290 16199298 G A); (22291 16199912 C T); (22292 16200063
A G); (22293 16201051 G A); (22294 16201448 T C); (22295 16201512 T C); (22296 16201876 T
C); (22297 16202087 C T); (22298 16202666 T C); (22299 16202692 C T); (22300 16202866 G A); (22301
16202917 G A); (22302 16203408 T C); (22303 16203990 G A); (22304 16205045 A T); (22305 16205393
G T); (22306 16206673 C T); (22307 16207211 A G); (22308 16208589 G A); (22309 16208794 T
G); (22310 16208897 G A); (22311 16208947 A G); (22312 16209745 C T); (22313 16209910 T C); (22314
16210119 T C); (22315 16210121 A G); (22316 16210210 T G); (22317 16210240 C T); (22318 16210273
A G); (22319 16210287 T C); (22320 16211463 C G); (22321 16211781 C T); (22322 16212508 C T); (22323
16212842 G GA); (22324 16212952 T A); (22325 16212979 T A); (22326 16214705 A G); (22327
16215764 T A); (22328 16216603 A G); (22329 16217188 A G); (22330 16217189 T G); (22331 16217190
A T); (22332 16217191 T A); (22333 16217192 A C); (22334 16217193 T G); (22335 16217195 T A); (22336
16217930 G A); (22337 16218875 T C); (22338 16218875 TATAC T); (22339 16219053 A AT); (22340
16220128 A G); (22341 16221044 C G); (22342 16222217 G A); (22343 16223033 C G); (22344 16223416
C T); (22345 16223495 G A); (22346 16223625 C T); (22347 16224098 G C); (22348 16225819 G
T); (22349 16227272 G A); (22350 16227848 T G); (22351 16228084 G T); (22352 16228090 A T); (22353
16228746 A G); (22354 16229926 A G); (22355 16230210 GA G); (22356 16230548 C T); (22357
16230904 A G); (22358 16231151 T C); (22359 16231169 G A); (22360 16231387 A G); (22361 16233615
G A); (22362 16234224 T TTA); (22363 16235161 C T); (22364 16236303 A AT); (22365 16236453 G
A); (22366 16236676 A G); (22367 16236776 T C); (22368 16238251 G A); (22369 16238455 G A); (22370
16238686 C A); (22371 16239013 C A); (22372 16239037 T C); (22373 16239969 T C); (22374 16240046
A C); (22375 16240380 G A); (22376 16240600 G C); (22377 16240630 T G); (22378 16240731 G
A); (22379 16242641 G T); (22380 16243098 G A); (22381 16243779 T G); (22382 16243798 T G); (22383
16244293 C T); (22384 16244345 G A); (22385 16244658 GA G); (22386 16245899 A G); (22387
16246249 A G); (22388 16246484 C A); (22389 16248610 C T); (22390 16249261 T A); (22391 16250022
A G); (22392 16250434 A C); (22393 16250713 G T); (22394 16251322 A G); (22395 16251462 G
C); (22396 16251536 G A); (22397 16251655 T G); (22398 16251688 C A); (22399 16252492 A G); (22400
16252662 A G); (22401 16253918 C T); (22402 16254005 T C); (22403 16254296 T C); (22404 16254962
A G); (22405 16256433 C A); (22406 16256668 T C); (22407 16256796 C A); (22408 16256877 T
C); (22409 16257550 G A); (22410 16258105 A C); (22411 16258254 C A); (22412 16258333 A C); (22413
16258360 A G); (22414 16259054 C A); (22415 16259635 G T); (22416 16260186 G A); (22417 16260493
T C); (22418 16260566 G A); (22419 16261429 A G); (22420 16261521 A G); (22421 16262022 C
G); (22422 16262113 C T); (22423 16262434 A C); (22424 16262609 A G); (22425 16263061 A G); (22426
16263280 C T); (22427 16264102 T C); (22428 16265266 A G); (22429 16265453 C G); (22430 16266239
T A); (22431 16266245 C T); (22432 16266592 AT A); (22433 16267717 C T); (22434 16267771 G
T); (22435 16267854 C T); (22436 16268102 T C); (22437 16269260 C T); (22438 16269868 T C); (22439
16269911 T A); (22440 16270512 G A); (22441 16272024 G A); (22442 16272276 C T); (22443 16272517
A C); (22444 16273854 C A); (22445 16274009 G A); (22446 16274115 T C); (22447 16274125 G
A); (22448 16274130 C A); (22449 16275620 A G); (22450 16275782 T C); (22451 16276671 C T); (22452
16276977 C T); (22453 16277582 A G); (22454 16277642 C T); (22455 16277710 T C); (22456 16278197
G A); (22457 16278937 A G); (22458 16279887 C T); (22459 16280195 T G); (22460 16280830 T
C); (22461 16280987 G A); (22462 16281069 C G); (22463 16282070 G A); (22464 16282813 T C); (22465
16282911 C T); (22466 16283841 C G); (22467 16283843 A ACATAT); (22468 16283886 T G); (22469
16285470 C T); (22470 16285658 C T); (22471 16285790 C T); (22472 16285866 A G); (22473 16285957
A G); (22474 16286694 C T); (22475 16286761 C T); (22476 16287122 G A); (22477 16287376 T
C); (22478 16287548 G A); (22479 16287619 C A); (22480 16287835 C T); (22481 16287842 G T); (22482
16288892 G A); (22483 16288989 G A); (22484 16289003 G A); (22485 16289363 G C); (22486 16289569
C T); (22487 16289589 G T); (22488 16289601 A G); (22489 16290508 G A); (22490 16290811 T
TA); (22491 16291429 G A); (22492 16291497 G A); (22493 16291592 A G); (22494 16291594 T
C); (22495 16292001 A G); (22496 16292067 C T); (22497 16292880 A G); (22498 16293320 T C); (22499
16293800 A C); (22500 16294270 G C); (22501 16295061 G A); (22502 16296337 G A); (22503 16297175
G A); (22504 16297905 T C); (22505 16297958 C T); (22506 16297960 C T); (22507 16297961 G
A); (22508 16297962 G T); (22509 16297964 G T); (22510 16299089 A G); (22511 16299169 A G); (22512
16299329 T C); (22513 16302139 A C); (22514 16303331 G A); (22515 16303441 C T); (22516 16303739
T C); (22517 16304400 C T); (22518 16304438 G A); (22519 16305263 G A); (22520 16305272 C
A); (22521 16305303 C G); (22522 16307079 A T); (22523 16307080 G T); (22524 16307081 G A); (22525
16307082 T A); (22526 16307105 ACCAAGAGGGGGGGTGAATTGGT A); (22527 16307337 A G); (22528
16308424 T G); (22529 16308696 G A); (22530 16308851 G A); (22531 16309314 A G); (22532 16309327
A C); (22533 16310726 A C); (22534 16310853 T C); (22535 16310965 C T); (22536 16311463 G C); (22537
16312017 G T); (22538 16312406 C T); (22539 16312476 G A); (22540 16313467 C T); (22541 16314463
G T); (22542 16315589 G C); (22543 16317594 TA T); (22544 16317846 G A); (22545 16318094 A
G); (22546 16319785 T A); (22547 16319814 C T); (22548 16320127 G A); (22549 16320244 T A); (22550
16320497 AT A); (22551 16322375 A G); (22552 16322753 C T); (22553 16323622 G T); (22554 16324505
T G); (22555 16325297 T C); (22556 16325777 A G); (22557 16326261 T G); (22558 16326339 C
T); (22559 16327097 C T); (22560 16328781 C T); (22561 16328984 T A); (22562 16329073 A G); (22563
16329495 T C); (22564 16329617 G T); (22565 16329622 A T); (22566 16330090 A G); (22567 16330212
G A); (22568 16330821 T C); (22569 16330934 AT A); (22570 16331288 G T); (22571 16331492 G
T); (22572 16332074 G A); (22573 16332286 A G); (22574 16332354 T C); (22575 16333967 A G); (22576
16334036 G A); (22577 16334301 T C); (22578 16334304 G T); (22579 16334399 G A); (22580 16334738
C T); (22581 16334997 A G); (22582 16335319 A ATAATT); (22583 16336241 C T); (22584 16337280 AT
A); (22585 16337504 CT C); (22586 16338711 G A); (22587 16339047 G A); (22588 16339186 C A); (22589
16339305 G A); (22590 16340060 T G); (22591 16340319 C T); (22592 16340818 T C); (22593 16340833
T TGG); (22594 16341093 C T); (22595 16341096 C T); (22596 16341231 C T); (22597 16341429 T
C); (22598 16341637 A G); (22599 16342129 G GA); (22600 16342234 T G); (22601 16344788 T
C); (22602 16346434 A G); (22603 16346651 G A); (22604 16346949 C T); (22605 16347024 C A); (22606
16347090 G A); (22607 16347838 C A); (22608 16348127 AT A); (22609 16348359 T A); (22610
16349586 C T); (22611 16349599 C A); (22612 16350151 T C); (22613 16351432 A G); (22614 16352201
G A); (22615 16352309 G C); (22616 16352737 C T); (22617 16353034 C A); (22618 16353585 C
G); (22619 16355197 A C); (22620 16355308 G T); (22621 16355575 A T); (22622 16356099 C T); (22623
16356139 C T); (22624 16356237 A C); (22625 16356287 T G); (22626 16357132 G A); (22627 16358806
C T); (22628 16359199 A G); (22629 16360630 A T); (22630 16360655 T G); (22631 16362829 C T); (22632
16363408 C A); (22633 16364335 A G); (22634 16364348 G A); (22635 16364631 T C); (22636 16365110
G T); (22637 16365790 T A); (22638 16365878 C T); (22639 16365894 T C); (22640 16365911 G T); (22641
16365913 C T); (22642 16365958 T C); (22643 16366634 C T); (22644 16366997 C T); (22645 16367534 T
C); (22646 16367708 GA G); (22647 16368191 C A); (22648 16368378 G GT); (22649 16369607 C
G); (22650 16370072 C T); (22651 16370096 C T); (22652 16370136 A T); (22653 16370774 T C); (22654
16371386 G A); (22655 16372751 T G); (22656 16373376 G A); (22657 16373480 T C); (22658 16375000
G A); (22659 16375982 T C); (22660 16376363 C T); (22661 16376459 C T); (22662 16376944 A
G); (22663 16377280 C T); (22664 16377694 G A); (22665 16378235 T C); (22666 16378257 G C); (22667
16378794 G A); (22668 16379068 G A); (22669 16379178 T G); (22670 16379615 C T); (22671 16379760
C T); (22672 16380256 T C); (22673 16381618 G A); (22674 16381814 C G); (22675 16384027 G
A); (22676 16384243 G A); (22677 16384244 T G); (22678 16384347 G A); (22679 16384354 T C); (22680
16385240 C A); (22681 16385442 T C); (22682 16385477 G A); (22683 16388827 T C); (22684 16389509
G A); (22685 16390208 C T); (22686 16391038 A G); (22687 16391096 A G); (22688 16391892 A
AT); (22689 16392708 G A); (22690 16393563 G T); (22691 16394255 T TGATTAAAATTACTC); (22692
16394508 G T); (22693 16394546 A G); (22694 16395179 A G); (22695 16395488 G A); (22696 16395975
T C); (22697 16396249 G C); (22698 16396730 T A); (22699 16397675 G A); (22700 16399574 G
A); (22701 16399748 T C); (22702 16399755 C A); (22703 16400338 T C); (22704 16400726 G C); (22705
16401009 TA T); (22706 16401114 A G); (22707 16401332 A C); (22708 16401389 C T); (22709 16402267
C T); (22710 16402292 A T); (22711 16402451 C A); (22712 16402758 G A); (22713 16403660 G
T); (22714 16404162 A C); (22715 16404515 T TATATAG); (22716 16405079 A G); (22717 16405278 C
T); (22718 16407184 A G); (22719 16407642 C T); (22720 16407892 C G); (22721 16408687 C A); (22722
16409006 G A); (22723 16409749 T G); (22724 16409842 C T); (22725 16409962 T C); (22726 16409978
T A); (22727 16410216 A G); (22728 16410233 A G); (22729 16411063 T C); (22730 16411086 T
C); (22731 16411126 C T); (22732 16411138 C T); (22733 16411167 G C); (22734 16414596 C A); (22735
16415063 G A); (22736 16417000 C T); (22737 16418372 G T); (22738 16421051 A T); (22739 16421130
G A); (22740 16422652 G A); (22741 16423037 G C); (22742 16423423 G A); (22743 16423796 C
T); (22744 16423905 G C); (22745 16424064 T C); (22746 16424178 G A); (22747 16424308 C T); (22748
16424701 G C); (22749 16424750 C T); (22750 16425195 A G); (22751 16425229 C T); (22752 16425260
A AT); (22753 16426692 A G); (22754 16426700 T G); (22755 16427934 C T); (22756 16428052 A
G); (22757 16428088 C A); (22758 16434266 G A); (22759 16435066 CAGCATTT C); (22760 16435067 G
A); (22761 16436223 T TTTTTCTTC); (22762 16436763 C A); (22763 16436870 C T); (22764 16437169 G
C); (22765 16437604 C A); (22766 16437825 C T); (22767 16438090 G A); (22768 16439243 G T); (22769
16440184 C T); (22770 16441812 G T); (22771 16442993 A G); (22772 16445254 T G); (22773 16445338
T C); (22774 16445765 G GTTGACTACCTAACAATTCTTGTAAGGTACA); (22775 16447231 T C); (22776
16447446 A C); (22777 16447809 T C); (22778 16449381 A C); (22779 16449463 A G); (22780 16450508
C T); (22781 16451004 G C); (22782 16451710 C T); (22783 16451780 G A); (22784 16451868 G
A); (22785 16452103 G A); (22786 16452886 G A); (22787 16453417 G A); (22788 16454050 C T); (22789
16454828 G T); (22790 16454860 T C); (22791 16455312 A G); (22792 16455385 C T); (22793 16455404
A C); (22794 16455806 A G); (22795 16455874 A C); (22796 16456138 A G); (22797 16457168 C
T); (22798 16457522 A G); (22799 16457697 C A); (22800 16457924 G T); (22801 16458148 T C); (22802
16458416 T C); (22803 16458417 A T); (22804 16458418 G A); (22805 16458650 C T); (22806 16459059
C T); (22807 16459889 G C); (22808 16460386 A G); (22809 16460515 T C); (22810 16460625 G
A); (22811 16460681 C T); (22812 16461122 G A); (22813 16461962 G A); (22814 16462000 A G); (22815
16462027 C T); (22816 16462146 A G); (22817 16462627 G A); (22818 16463473 A G); (22819 16463585
G A); (22820 16464140 G A); (22821 16464481 G A); (22822 16465142 G A); (22823 16465555 G
A); (22824 16465704 C T); (22825 16465720 C A); (22826 16466600 G A); (22827 16467575 G A); (22828
16468280 C T); (22829 16468379 A C); (22830 16468740 C T); (22831 16470254 T C); (22832 16470258 A
T); (22833 16471124 G A); (22834 16471258 A G); (22835 16472826 T A); (22836 16473133 T G); (22837
16473805 T C); (22838 16473827 T C); (22839 16474721 G C); (22840 16476343 T G); (22841 16476745
T C); (22842 16476797 C A); (22843 16476968 G A); (22844 16477390 T C); (22845 16477546 A T); (22846
16477830 C A); (22847 16478424 C T); (22848 16478797 AT A); (22849 16479546 G A); (22850 16479807
G A); (22851 16480424 C T); (22852 16481650 T C); (22853 16482366 T C); (22854 16482684 T
TC); (22855 16482896 G T); (22856 16483148 T G)

### Example 3: Introgression of R-Gene Interval Into Glycine max Lines

Known methods of introgression from wild glycine species involve doubled F1 plants (FiD). However, such methods tend to be inefficient with a low number of infertile hybrids being produced. Further, few hybrids survive the subsequent chromosome doubling process wherein the chromosomes of the infertile hybrid are doubled by a chemical agent (typically colchicine) to make it fertile.

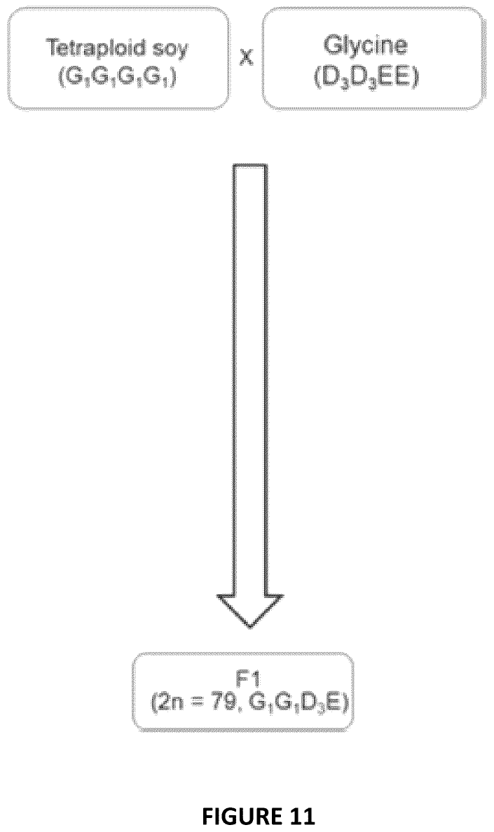

In another method of introgression from wild glycine species, as described below and illustrated at FIG. 11, Applicants introgressed the ASR resistance trait using tetraploid soy. Briefly, the method of introgression using tetraploid soy involves doubling a domestic G. max genome to make it more compatible and efficient for crossing with a wild Glycine genome. The method allows for the efficient production of fertile hybrids that can be further backcrossed to move desirable genes and traits from wild glycine into domestic and elite G. max soybean lines, without the need for artificial genetic modification or gene editing.

Doubled soy lines (tetraploid soy) were generated from two ASR susceptible G. max elite lines, herein referred to as Female 1 and Female 2 (two Syngenta proprietary lines). The double susceptible G. max line had 2n=40 chromosomes (GiGi genome) and after doubling is in a tetraploid state as 4n=80. In comparison, the G. tomentella resistant parent has 2n=78 or 40 chromosomes (D₃E₁ or D genome, respectively). For the doubling, immature soybean embryos of the G. max lines in tissue culture medium were treated with approximately 0.25-1.0 Omg/ml colchicine for 3-4 days at 25° C. Regenerated plants were transferred to soil, and leaf samples were taken for ploidy analysis to confirm chromosome doubling. Tetraploid plants were allowed to self, and ploidy analysis was performed on embryos to confirm doubling. An unlimited seed supply was produced by allowing the tetraploid soy to self.

Flower buds prior to anthesis were prepared for the doubled female lines by gently removing sepals and petals to expose the mature stigma. Pollen from freshly opened flowers of G. tomentella (2n=78 or 40) was obtained by gently removing the petals to expose the mature anthers and dusting the pollen onto the soybean stigma. The crosses are shown at Table 3.

TABLE 3
Plant Crossings

| | Male species and | | Female species and |
|---|---|---|---|
| PI# (male) | 2n Chromosome # | PI # (female) | 2n Chromosome # |
| PI505267 | G. tomentella; | Female 1 | G. max; 2n = 40 |
| | 2n = 40 | | |
| PI505267 | G. tomentella; | Female 2 | G. max; 2n = 40 |
| | 2n = 40 | | |

Dicamba, a synthetic auxin herbicide (FeXapan, Corteva Agriscience, Wilmington, DE), was sprayed on the tetraploid x Glycine crosses to produce pod and embryo formation. Dicamba was sprayed at a 3 to 20 mg/L concentration. A spray bottle or atomizer was used to achieve good saturation of the pollinated gynoecia and the node to which it was attached.

Multiple B1 embryos and a B1 plant was generated by crossing the doubled susceptible G. max parent with the resistant G. tomentella parent (these plants would have been the FlD plants if standard introgression was used). The cross was then verified via TaqMan assays (Applied Biosystems, Waltham, MA). In one particular example, the TaqMan assays depicted at Table 4 were used to confirm the presence of the chromosomal interval associated with ASR resistance (that is, the interval comprising SEQ ID NO: 1) in the hybrid plants.

TABLE 4
Taqman data to confirm hybrids

| | TaqMan | TaqMan | TaqMan | TaqMan | TaqMan | TaqMan |
|---|---|---|---|---|---|---|
| | 3289 | 3316 | 3434 | 3435 | 3537 | 3538 |
| | Copy# | Copy# | Copy# | Copy# | Copy# | Copy# |
| Cross | level | level | level | level | level | level |
| Female only | 0 | 0 | 0 | 0 | 0 | 0 |
| Female 1 × | 0 | 1 | 1 | 0 | 1 | 0 |
| PI505267 | | | | | | |

It is well known in the art that given the sequence and the SNP allele associated with a given trait (e.g. ASR resistance), one having ordinary skill in the art could develop oligonucleotide primers and use said primers to identify plants carrying the chromosomal interval depicted in SEQ ID NO: 1, or a functional fragment thereof comprising the causative gene. A TAQMAN® assay (e.g. generally a two-step allelic discrimination assay or similar), a KASP™ assay (generally a one-step allelic discrimination assay defined below or similar), or both can be employed to assay the SNPs as disclosed herein. In an exemplary two-step assay, a forward primer, a reverse primer, and two assay probes (or hybridization oligos; herein also referred to as assay primers or assay probes) are employed (see SEQ ID NOs: 22-237 detailed at Tables 5-6). The forward and reverse primers are employed to amplify genetic loci that comprise SNPs that are associated with ASR resistance loci. The particular nucleotides that are present at the SNP positions are then assayed using the assay primer, which in each pair differ from each other with respect to the nucleotides that are present at the SNP position (although it is noted that in any given pair, the primers can differ in their 5′ or 3′ ends without impacting their abilities to differentiate between nucleotides present at the corresponding SNP positions). In some embodiments, each pair of assay primers are differentially labeled with, for example, fluorophores to permit distinguishing between the two assay probes in a single reaction. In some embodiments, the assay primers and the reaction conditions are designed such that an assay primer will only hybridize to the reverse complement of a 100% perfectly matched sequence, thereby permitting identification of which allele(s) is/are present based upon detection of hybridizations.

Table 5 provides a list of example assay IDs, wherein each assay ID corresponds to a particular SNP position within the chromosomal interval represented by SEQ ID NO: 1. The assays are designed to differentiate between favorable and unfavorable alleles associated with a given SNP position, as indicated.

Table 6 provides a list and sequence of the assay components used in each of the assays listed in Table 5. Particularly, Table 6 lists the sequences of the specific forward and reverse primers as well as the sequence and combination of fluorophores used for each of the assays. In the listing of the assay components, the assay component ID indicates the associated assay ID (Table 5) and the nature of the component (whether it is a probe or a primer). The suffix F2 indicates that the corresponding sequence is for a forward primer, the suffix R1 indicates that the corresponding sequence is for a forward primer, the suffix FM indicates that the corresponding sequence is for an assay probe having the FAM fluorophore, and the suffix TT indicates that the corresponding sequence is for an assay probe having the TET fluorophore. For example, “S21399A1FM”, “S21399A1TT”, “S21399F2” and “S21399R1” refer, respectively, to the FAM probe, TET probe, forward primer, and reverse primer for Assay ID S21399 used for identification of the allele corresponding to the SNP at position 10832017.

TABLE 5
Assays associated with SNP positions within SEQ ID NO:
1 that are associated with increased resistance to ASR

| | Favorable Allele | Unfavorable allele | |
|---|---|---|---|
| Position | (Rust Resistant) | (Rust Susceptible) | Assay ID |
| 9673275 | A | G | S21473 |
| 10442099 | C | T | S21899 |
| 10458451 | G | T | S21868 |
| 10577275 | G | T | S21574 |
| 10584306 | G | A | S21546 |
| 10653990 | T | C | S21524 |
| 10789947 | C | T | S21595 |
| 10790147 | A | T | S21550 |
| 10791059 | A | G | S21410 |
| 10791213 | G | A | S21484 |
| 10791310 | T | C | S21425 |
| 10791380 | T | A | S21556 |
| 10791581 | G | A | S21416 |
| 10832017 | C | T | S21399 |
| 10832133 | C | T | S21435 |
| 10832300 | G | A | S21504 |
| 10832584 | G | A | S21470 |
| 10833393 | T | C | S21495 |
| 10867232 | T | G | S21525 |
| 10867366 | A | G | S21593 |
| 10867536 | G | A | S21406 |
| 10875079 | G | A | S21516 |
| 10875110 | C | T | S21466 |
| 10875181 | C | T | S21421 |
| 10875235 | G | T | S21472 |
| 10881334 | G | C | S21587 |
| 10881474 | A | G | S21559 |
| 10881948 | A | C | S21530 |
| 10892735 | A | G | S21500 |
| 10892762 | G | T | S21536 |
| 10892926 | C | T | S21584 |
| 10892975 | A | G | S21468 |
| 10892980 | T | C | S21572 |
| 10892981 | A | G | S21509 |
| 10893019 | A | G | S21471 |
| 10907830 | T | C | S21443 |
| 10907844 | G | A | S21420 |
| 10907888 | T | C | S21441 |
| 10907918 | A | G | S21520 |
| 10916866 | T | C | S21422 |
| 11641302 | C | T | S21911 |
| 12396655 | A | G | S21459 |
| 12556909 | C | T | S21407 |
| 12677703 | T | G | S21527 |
| 12689190 | A | G | S21508 |
| 12830968 | T | C | S21463 |
| 12931808 | G | A | S21505 |
| 12940915 | A | T | S21446 |
| 13106765 | G | A | S21476 |
| 13605770 | C | A | S21442 |
| 13658793 | C | T | S21562 |
| 15286080 | T | C | S21497 |
| 15315501 | C | T | S21512 |
| 15586799 | A | G | S21454 |

TABLE 6
Sequences of assay components used in assays associated with SNP positions
within SEQ ID NO: 1. Suffix “F2” refers to a forward primer; Suffix
“R1” refers to a reverse primer. Primers with a common prefix can
form a primer pair. Suffix FM and TT refer to probes.

| Assay | | | | |
|---|---|---|---|---|
| component | DNA sequence | SEQ ID NO. | Fluor. | Allele |
| S21399A1FM | TCGAGCTCTGGTGTC | SEQ ID NO: 2 | FAM | G |
| S21399A2TT | CCTCGAGCTTTGGTGT | SEQ ID NO: 3 | TET | A |
| S21399F2 | GAGACCCATTGCGTGGAAAG | SEQ ID NO: 4 | | |
| S21399R1 | CAACGGCGTTAAGGAATACCCT | SEQ ID NO: 5 | | |
| S21406A1FM | CTTCAGCCTCATCGG | SEQ ID NO: 6 | FAM | G |
| S21406A2TT | TCTTCAGCTTCATCGG | SEQ ID NO: 7 | TET | A |
| S21406F2 | GACGCCCGTGCAGTAGAG | SEQ ID NO: 8 | | |
| S21406R1 | AGTGCTCCTAAGGTGTTGCCT | SEQ ID NO: 9 | | |
| S21407A1FM | CTTTAATCCCCACAAACTG | SEQ ID NO: 10 | FAM | A |
| S21407A2TT | TTAATCCCCGCAAACTG | SEQ ID NO: 11 | TET | G |
| S21407F1 | CAGCTCATGCTCATAGTCCTCA | SEQ ID NO: 12 | | |
| S21407R2 | AAGACCACCGTCCTCACTG | SEQ ID NO: 13 | | |
| S21410A1FM | CCCCGTTTTCACTTTAAA | SEQ ID NO: 14 | FAM | G |
| S21410A2TT | CCCCCGTTTTTACTTTAA | SEQ ID NO: 15 | TET | A |
| S21410F1 | GAGGCGATACGGCCAATCAG | SEQ ID NO: 16 | | |
| S21410R2 | CCTCCATATCCTCCCTTGTGAAG | SEQ ID NO: 17 | | |
| S21416A1FM | CGATTCTCCGGGTACA | SEQ ID NO: 18 | FAM | G |
| S21416A2TT | AGCTCGATTCTCTGGGTA | SEQ ID NO: 19 | TET | A |
| S21416F2 | GGAACTGGAGCAAATGGAGGTT | SEQ ID NO: 20 | | |
| S21416R1 | GGTCGAGGTTTCATGTCGCTAT | SEQ ID NO: 21 | | |
| S21420A1FM | TCATGGACCCGTCTTT | SEQ ID NO: 22 | FAM | G |
| S21420A2TT | TTTCATGGACCTGTCTTT | SEQ ID NO: 23 | TET | A |
| S21420F2 | CTTAGAATCTTGTGCGGTGACAAAT | SEQ ID NO: 24 | | |
| S21420R1 | CAAACTCGTGCTTTCAGATTCAGT | SEQ ID NO: 25 | | |
| S21421A1FM | CTACTAACAAATTATAAGGCACT | SEQ ID NO: 26 | FAM | A |
| S21421A2TT | ACTAACAAATTATGAGGCACT | SEQ ID NO: 27 | TET | G |
| S21421F1 | CCTCTTTGTGAGTCGTGTCCAA | SEQ ID NO: 28 | | |
| S21421R2 | TCAGTCTCTCTCCTCAACTACCTT | SEQ ID NO: 29 | | |
| S21422A1FM | CAGGTGGCACAACATCA | SEQ ID NO: 30 | FAM | A |
| S21422A2TT | AGGTGGCACGACATCAC | SEQ ID NO: 31 | TET | G |
| S21422F1 | CTCCAGCCGTAGGATGACTT | SEQ ID NO: 32 | | |
| S21422R2 | GGAGGCAGTTAGTGAAGAATGTG | SEQ ID NO: 33 | | |
| S21425A1FM | CGAGAAGGCCGACTGA | SEQ ID NO: 34 | FAM | G |
| S21425A2TT | TCCGAGAAGGCTGACTG | SEQ ID NO: 35 | TET | A |
| S21425F1 | CCTGTGTCCGGATCACATACTG | SEQ ID NO: 36 | | |
| S21425R2 | TTGCGAGATGCAATTGAGAAGT | SEQ ID NO: 37 | | |
| S21435A1FM | AATTCCAACCCCATCTC | SEQ ID NO: 38 | FAM | G |
| S21435A2TT | AATTCCAACTCCATCTCA | SEQ ID NO: 39 | TET | A |
| S21435F2 | GAGGAGGTGGTCCTAGGGTAT | SEQ ID NO: 40 | | |
| S21435R1 | CCCGGAACATAAGTCTCTACAAGA | SEQ ID NO: 41 | | |
| S21441A1FM | CACCGCACAAGATTCTA | SEQ ID NO: 42 | FAM | A |
| S21441A2TT | CACCGCACAGGATTCT | SEQ ID NO: 43 | TET | G |
| S21441F1 | CAGTTTTCATGGACCCGTCTTTG | SEQ ID NO: 44 | | |
| S21441R2 | AGTGGATCCTAAACCCTAAACACA | SEQ ID NO: 45 | | |
| S21442A1FM | TTGGGAGGGATGGGAGGT | SEQ ID NO: 46 | FAM | C |
| S21442A2TT | CTTGGGAGGGATTGGAGGT | SEQ ID NO: 47 | TET | A |
| S21442F2 | CAAGGAGGTGGGTTGGACTATAAG | SEQ ID NO: 48 | | |
| S21442R1 | GCCAGCTGCAACATAGCCT | SEQ ID NO: 49 | | |
| S21443A1FM | TCCATGAAAACCGAATCTG | SEQ ID NO: 50 | FAM | G |
| S21443A2TT | TCCATGAAAACTGAATCTGA | SEQ ID NO: 51 | TET | A |
| S21443F2 | TGAAACGTCCAGGTACAACGAAT | SEQ ID NO: 52 | | |
| S21443R1 | CTTAGAATCTTGTGCGGTGACA | SEQ ID NO: 53 | | |
| S21446A1FM | CTCTCCCTTTAATTGATCA | SEQ ID NO: 54 | FAM | T |
| S21446A2TT | CTCTCCCTTTTATTGATCA | SEQ ID NO: 55 | TET | A |
| S21446F1 | TGATTTACCTGCCAACCTCTCTA | SEQ ID NO: 56 | | |
| S21446R2 | AAGCCACTCGCAAGGACCATT | SEQ ID NO: 57 | | |
| S21454A1FM | ACCCTATGGACTCCTACAG | SEQ ID NO: 58 | FAM | G |
| S21454A2TT | CCTATGGACTCTTACAGC | SEQ ID NO: 59 | TET | A |
| S21454F1 | TCGAGTCGGGAATGGGTAGAG | SEQ ID NO: 60 | | |
| S21454R2 | GAAAGCCAAACCTTCAAACCAC | SEQ ID NO: 61 | | |
| S21459A1FM | AGGGATGACTATGCTGTTATAG | SEQ ID NO: 62 | FAM | G |
| S21459A2TT | AGGAGGGATGACTATGTTGTTA | SEQ ID NO: 63 | TET | A |
| S21459F1 | GGACTCTTGGAGACTATGCTTATCA | SEQ ID NO: 64 | | |
| S21459R2 | GGCTTCAGTTCCACAACCTTATTG | SEQ ID NO: 65 | | |
| S21463A1FM | CAATTTGGCGACTGCAT | SEQ ID NO: 66 | FAM | A |
| S21463A2TT | AATTTGGCGGCTGCATG | SEQ ID NO: 67 | TET | G |
| S21463F2 | GCATCACACATTAGCTCAAAGGG | SEQ ID NO: 68 | | |
| S21463R1 | TGCAAAGAGGCATTTAAGGGATT | SEQ ID NO: 69 | | |
| S21466A1FM | TTTGTGAGTCATGTCCAA | SEQ ID NO: 70 | FAM | A |
| S21466A2TT | TGTGAGTCGTGTCCAA | SEQ ID NO: 71 | TET | G |
| S21466F2 | GCCGAATGGATCACATGTCCT | SEQ ID NO: 72 | | |
| S21466R1 | TCCTCAACTACCTTTTCCCCAAA | SEQ ID NO: 73 | | |
| S21468A1FM | AGACAACAATGTCAATGAGTCT | SEQ ID NO: 74 | FAM | A |
| S21468A2TT | ACAACAATGTCAGTGAGTCT | SEQ ID NO: 75 | TET | G |
| S21468F2 | GTCACTCCATCCATATCTAGCCTT | SEQ ID NO: 76 | | |
| S21468R1 | GAAGCCGTTGTATCCGAGGTG | SEQ ID NO: 77 | | |
| S21470A1FM | CAGGCTCACATCCTAC | SEQ ID NO: 78 | FAM | A |
| S21470A2TT | AGGCTCACGTCCTACA | SEQ ID NO: 79 | TET | G |
| S21470F1 | CGTGGCTACTACCACCAGAGAT | SEQ ID NO: 80 | | |
| S21470R2 | TCATGTGAACGACTTAGGACCAT | SEQ ID NO: 81 | | |
| S21471A1FM | CCATCCATATCTAGCCTT | SEQ ID NO: 82 | FAM | A |
| S21471A2TT | CCATATCTGGCCTTGAC | SEQ ID NO: 83 | TET | G |
| S21471F1 | GTACCTCAAGCAGTGAACTGAA | SEQ ID NO: 84 | | |
| S21471R2 | GAGGTGCAAAGACTCATTGACA | SEQ ID NO: 85 | | |
| S21472A1FM | TGTGTAGTAGGGTGATTAAATT | SEQ ID NO: 86 | FAM | C |
| S21472A2TT | TATTGTGTAGTAGGGTTATTAAATT | SEQ ID NO: 87 | TET | A |
| S21472F1 | TGCCGAATGGATCACATGTCCT | SEQ ID NO: 88 | | |
| S21472R2 | TCCTCAACTACCTTTTCCCCAAA | SEQ ID NO: 89 | | |
| S21473A1FM | TTGGTCTTGTGAACATAGG | SEQ ID NO: 90 | FAM | G |
| S21473A2TT | TTTGGTCTTGTGAATATAGGA | SEQ ID NO: 91 | TET | A |
| S21473F2 | CCGGAGGATGAACTGATGGA | SEQ ID NO: 92 | | |
| S21473R1 | TGGTGGCCTTACAATGCTG | SEQ ID NO: 93 | | |
| S21476A1FM | TGCCACCGAAGTGC | SEQ ID NO: 94 | FAM | G |
| S21476A2TT | CTTGCCACTGAAGTGC | SEQ ID NO: 95 | TET | A |
| S21476F1 | GGAGGCTCCAAGATCACATAAGG | SEQ ID NO: 96 | | |
| S21476R2 | TGATGCCATCCCACTATCCA | SEQ ID NO: 97 | | |
| S21484A1FM | AAGAGACCCCATGAAGCC | SEQ ID NO: 98 | FAM | A |
| S21484A2TT | AGACCCCGTGAAGCC | SEQ ID NO: 99 | TET | G |
| S21484F1 | CAACATGACAAAAGAGATGAGGGA | SEQ ID NO: 100 | | |
| S21484R2 | GGTTCCGGGAATATGTCGTTATC | SEQ ID NO: 101 | | |
| S21495A1FM | TGTCAACACTCAGGTGA | SEQ ID NO: 102 | FAM | G |
| S21495A2TT | CCTGTCAACACTTAGGTG | SEQ ID NO: 103 | TET | A |
| S21495F1 | GCCTCCTCCTTGGAGATGTTC | SEQ ID NO: 104 | | |
| S21495R2 | CAAGGAATCAAGGTCTCAAATCACA | SEQ ID NO: 105 | | |
| S21497A1FM | AGACCTCAACAGCAATATAATC | SEQ ID NO: 106 | FAM | A |
| S21497A2TT | CTCAACAGCAGTATAATCC | SEQ ID NO: 107 | TET | G |
| S21497F1 | CCATGCAACCTGACCAGTCATTC | SEQ ID NO: 108 | | |
| S21497R2 | GGACCAGGACCATACCTCAA | SEQ ID NO: 109 | | |
| S21500A1FM | AGAAGATGGTCCCATATGTCT | SEQ ID NO: 110 | FAM | A |
| S21500A2TT | AAGATGGTCCCGTATGTC | SEQ ID NO: 111 | TET | G |
| S21500F1 | TCACCATGTCATATCCATGTCGTA | SEQ ID NO: 112 | | |
| S21500R2 | TGCAATTTGCCTCCGAAAGAAG | SEQ ID NO: 113 | | |
| S21504A1FM | TAGAGCTGTTGACATGTTG | SEQ ID NO: 114 | FAM | G |
| S21504A2TT | TTAGAGCTGTTGATATGTTGT | SEQ ID NO: 115 | TET | A |
| S21504F1 | GACTCTGCTCAGGCTAAGACA | SEQ ID NO: 116 | | |
| S21504R2 | GGGCTGATTATATGGTTGCTATGA | SEQ ID NO: 117 | | |
| S21505A1FM | AAGATCGCGTTTTAGAGGAG | SEQ ID NO: 118 | FAM | A |
| S21505A2TT | CGCGTTTTGGAGGAGA | SEQ ID NO: 119 | TET | G |
| S21505F1 | GGTCGGTGCAGACTATATTAAAGGG | SEQ ID NO: 120 | | |
| S21505R2 | GGGTCATCAGGCAAGGTCAT | SEQ ID NO: 121 | | |
| S21508A1FM | TTTGGTTCCAAGTGTGGG | SEQ ID NO: 122 | FAM | A |
| S21508A2TT | ATTGATTTGGTTCCAGGTGTG | SEQ ID NO: 123 | TET | G |
| S21508F1 | CACCGGACAGAGGGATAGAGTT | SEQ ID NO: 124 | | |
| S21508R2 | CATGCGGTAAGGAGCTATGGA | SEQ ID NO: 125 | | |
| S21509A1FM | CTCATTGACACTGTTGTC | SEQ ID NO: 126 | FAM | G |
| S21509A2TT | AGACTCATTGACATTGTTGTC | SEQ ID NO: 127 | TET | A |
| S21509F2 | TGTCACTCCATCCATATCTAGCCT | SEQ ID NO: 128 | | |
| S21509R1 | GAAGCCGTTGTATCCGAGGT | SEQ ID NO: 129 | | |
| S21512A1FM | CCATCCATTCGAGCAG | SEQ ID NO: 130 | FAM | G |
| S21512A2TT | CATCCATTTGAGCAGTC | SEQ ID NO: 131 | TET | A |
| S21512F2 | CACAACCCACGCGGAGTC | SEQ ID NO: 132 | | |
| S21512R1 | CGTAGTTCCACGCGATCAGT | SEQ ID NO: 133 | | |
| S21516A1FM | AGACTTCAATGCCGAATGG | SEQ ID NO: 134 | FAM | G |
| S21516A2TT | CAAAGACTTCAATGTCGAATG | SEQ ID NO: 135 | TET | A |
| S21516F2 | TTGGACACGACTCACAAAGA | SEQ ID NO: 136 | | |
| S21516R1 | GTGTTCCTACAAGACAATGCCTCT | SEQ ID NO: 137 | | |
| S21520A1FM | CGACATCAAATCTAAGACAATT | SEQ ID NO: 138 | FAM | A |
| S21520A2TT | CGACATCAAATCTAGGACAAT | SEQ ID NO: 139 | TET | G |
| S21520F1 | AGTGGATCCTAAACCCTAAACACA | SEQ ID NO: 140 | | |
| S21520R2 | TTGTCACCGCACAAGATTCTAAG | SEQ ID NO: 141 | | |
| S21524A1FM | ACCACAGCTATCAGAAAAGA | SEQ ID NO: 142 | FAM | G |
| S21524A2TT | TAGGACCACAGCTATTAGAAA | SEQ ID NO: 143 | TET | A |
| S21524F1 | CCACCAAGCTACCTGATTCCAGAT | SEQ ID NO: 144 | | |
| S21524R2 | TGGGATGACCTTAAGAGAATGTTTC | SEQ ID NO: 145 | | |
| S21525A1FM | CTTTCGACGAAGATTCTCA | SEQ ID NO: 146 | FAM | A |
| S21525A2TT | TCGACGACGATTCTCA | SEQ ID NO: 147 | TET | C |
| S21525F2 | TTGCAGGTGTCGTTGTAAGTTG | SEQ ID NO: 148 | | |
| S21525R1 | AGCAGCGACAATGGCAGAG | SEQ ID NO: 149 | | |
| S21527A1FM | TGACCACAGCTCCAG | SEQ ID NO: 150 | FAM | C |
| S21527A2TT | TTGACCACATCTCCAGT | SEQ ID NO: 151 | TET | A |
| S21527F2 | CCGCTTGGAAGCATCACA | SEQ ID NO: 152 | | |
| S21527R1 | TGAGGAGTGTTTCTTGAGTTTGAA | SEQ ID NO: 153 | | |
| S21530A1FM | ACAGAGTACACATGAACTAC | SEQ ID NO: 154 | FAM | C |
| S21530A2TT | TACAGAGTACACATTAACTACT | SEQ ID NO: 155 | TET | A |
| S21530F2 | GCATTGTTGTGGCAGATATGTTTG | SEQ ID NO: 156 | | |
| S21530R1 | TCTCGAGGGCCTATTTCATTTGA | SEQ ID NO: 157 | | |
| S21536A1FM | TAAGTCTTCTGAATCCCA | SEQ ID NO: 158 | FAM | C |
| S21536A2TT | TATAAGTCTTCTTAATCCCA | SEQ ID NO: 159 | TET | A |
| S21536F1 | AGACCGGATAGGGATGGAAA | SEQ ID NO: 160 | | |
| S21536R2 | CCGTCTGCGTGTCTGTGAAT | SEQ ID NO: 161 | | |
| S21546A1FM | TTCAAAATGGTAGACACTCA | SEQ ID NO: 162 | FAM | A |
| S21546A2TT | TTCAAAATGGTGGACACTC | SEQ ID NO: 163 | TET | G |
| S21546F1 | GATGAAGTTGATAGGACACAGTGAA | SEQ ID NO: 164 | | |
| S21546R2 | AGTGAGAGCTTGATGGAATATGGA | SEQ ID NO: 165 | | |
| S21550A1FM | TGAGTTGTTACAAGCTGGAT | SEQ ID NO: 166 | FAM | A |
| S21550A2TT | TGAGTTGTTACATGCTGGAT | SEQ ID NO: 167 | TET | T |
| S21550F1 | GAACACAGGCATTACACAATCACA | SEQ ID NO: 168 | | |
| S21550R2 | GGACCATGACCACATTTGAAAGC | SEQ ID NO: 169 | | |
| S21556A1FM | CCGGGTCCATTGTGG | SEQ ID NO: 170 | FAM | T |
| S21556A2TT | CGGGTCCTTTGTGGAA | SEQ ID NO: 171 | TET | A |
| S21556F1 | GGTCGCATTCACCGAGATGA | SEQ ID NO: 172 | | |
| S21556R2 | CCAGCATTGGTCCGTGGTATG | SEQ ID NO: 173 | | |
| S21559A1FM | TTAGTTGGTTCAGCAAACA | SEQ ID NO: 174 | FAM | A |
| S21559A2TT | ATTAGTTGGTTCGGCAAAC | SEQ ID NO: 175 | TET | G |
| S21559F2 | TGCAAGAAACAATAGCCTACAAAGG | SEQ ID NO: 176 | | |
| S21559R1 | GCTCCCAGGCTAAGATAATGC | SEQ ID NO: 177 | | |
| S21562A1FM | ATGCTTTAAATTCGTTCCCA | SEQ ID NO: 178 | FAM | G |
| S21562A2TT | TGCTTTAAATTTGTTCCCAA | SEQ ID NO: 179 | TET | A |
| S21562F2 | TCCAAAGGCAGGCAATAGC | SEQ ID NO: 180 | | |
| S21562R1 | ACCACTCAGAATCTGTGCAACTTC | SEQ ID NO: 181 | | |
| S21572A1FM | ACTCATTGACATTGTTGTC | SEQ ID NO: 182 | FAM | A |
| S21572A2TT | AAGACTCATTGACGTTGTTGT | SEQ ID NO: 183 | TET | G |
| S21572F1 | GAAGCCGTTGTATCCGAGGT | SEQ ID NO: 184 | | |
| S21572R2 | TGTCACTCCATCCATATCTAGCCT | SEQ ID NO: 185 | | |
| S21574A1FM | TGAACCACGATGTCTTC | SEQ ID NO: 186 | FAM | A |
| S21574A2TT | TGAACCACGCTGTCTT | SEQ ID NO: 187 | TET | C |
| S21574F2 | GGCAACAACCTCACTAGATTCATTG | SEQ ID NO: 188 | | |
| S21574R1 | TGGTTGGTGAAGTCGTACTCT | SEQ ID NO: 189 | | |
| S21584A1FM | CACTCTCCAACGTATCA | SEQ ID NO: 190 | FAM | G |
| S21584A2TT | AATCACTCTCCAATGTATCA | SEQ ID NO: 191 | TET | A |
| S21584F2 | CGTGATCTGGGACATGAGTCT | SEQ ID NO: 192 | | |
| S21584R1 | ACCTCGGATACAACGGCTTCTTC | SEQ ID NO: 193 | | |
| S21587A1FM | AGGAATACAAGAGCAGAAGCTC | SEQ ID NO: 194 | FAM | G |
| S21587A2TT | AAGGAATACAAGAGGAGAAGCTC | SEQ ID NO: 195 | TET | C |
| S21587F2 | GGTTCAGCAAACAAAGGAGACA | SEQ ID NO: 196 | | |
| S21587R1 | AAGGACAGAGGAAGTATAGGAGGTG | SEQ ID NO: 197 | | |
| S21593A1FM | TACGATTCCTCTATCTTC | SEQ ID NO: 198 | FAM | G |
| S21593A2TT | TTCTACGATTCCTTTATCTT | SEQ ID NO: 199 | TET | A |
| S21593F2 | GCGTAGCCTTGGTGGATGAAG | SEQ ID NO: 200 | | |
| S21593R1 | GTCGCTGCTCCTGCTCTTG | SEQ ID NO: 201 | | |
| S21595A1FM | TCCGGAGAATACATCCAT | SEQ ID NO: 202 | FAM | A |
| S21595A2TT | CGGAGAATGCATCCATA | SEQ ID NO: 203 | TET | G |
| S21595F2 | GGGTGCATCGGGATTTGATTG | SEQ ID NO: 204 | | |
| S21595R1 | AGACCAGTTGGTCGATAATTCTTC | SEQ ID NO: 205 | | |
| S21868A1FM | TATATTTTTACACAATTTGGTG | SEQ ID NO: 206 | FAM | A |
| S21868A2TT | TATTTTTACACGATTTGGTG | SEQ ID NO: 207 | TET | G |
| S21868F1 | CACAAATGATGCTAAGGTGGTGATA | SEQ ID NO: 208 | | |
| S21868R2 | TGTTGCAGAAATGCGTTCCTC | SEQ ID NO: 209 | | |
| S21899A1FM | CGAACTGGCACGATTTG | SEQ ID NO: 210 | FAM | A |
| S21899A2TT | CTGGCGCGATTTGC | SEQ ID NO: 211 | TET | G |
| S21899F1 | TCGAGTTACATTCCCACATCTGAA | SEQ ID NO: 212 | | |
| S21899R2 | CGCGGTACTCACACGACTA | SEQ ID NO: 213 | | |
| S21911A1FM | CCAGTATGAGGTATTAGACC | SEQ ID NO: 214 | FAM | A |
| S21911A2TT | AGCCAGTATGAGGTGTTAGAC | SEQ ID NO: 215 | TET | G |
| S21911F1 | TGGCATGGCTCTATGAGATGA | SEQ ID NO: 216 | | |
| S21911R2 | CCCAGTTCTCTGGTTACTCAAC | SED ID NO: 217 | | |

Introgression of the R gene intervals into G. max can alternatively be achieved using embryo rescue and chemical doubling. Therein, first an infertile hybrid of G. max and G. tomentella must be produced, which is an inefficient process resulting in low numbers of infertile hybrids. Next, embryo rescue must be performed and chemical treatment applied in order to generate amphidiploid shoots. If the amphidiploid plants are fertile, they are used to backcross with G. max for several generations in order to gradually eliminate the perennial Glycine chromosomes.

In one example, a wide cross is performed wherein Elite Syngenta soybean lines (RM 3.7 to 4.8) are used as the females (pollen recipients) and the recited accession line of Glycine tomentella is used as the males or pollen donors. Next, flowers are selected from the glycine plant containing anthers at the proper developmental stage. New, fully opened, brightly colored flowers hold anthers with mature pollen. The pollen appears as loose, yellow dust. These flowers are removed from the glycine plant and taken to the soybean plant for pollination. Pollen from the Glycine plants is generally used within 30 minutes of flower removal. Soybean flower buds that are ready for pollination are identified and selected. A soybean flower bud is generally ready when it is larger in size when compared to an immature bud. The sepals of the soybean blossoms are lighter in color and the petals are just beginning to appear. First, a pair of fine-tipped tweezers are used to carefully detach the sepals from the flower bud to expose the outer set of petals. Then, gently grasping and removing the petals (5 in total) from the flower, the ring of stamens surrounding the pistil is exposed. Since the stigma is receptive to pollen 1 day before the anthers begin shedding pollen it is important to recognize the stage development of “female ready, male not ready”. When pollinating soybean flowers at this developmental stage it is not necessary to emasculate the female flower. Next, the stigma is located on the soybean flower. Then using 1 male flower, the petals are carefully peeled off to expose the anthers and the pollen grains are gently dusted onto the stigma of the soybean flower. Care is taken not to damage the stigma at any time during this process. Starting the day after pollination, a hormone mixture is sprayed onto the pollinated flower and eventual developing F1 pod one time every day until harvest. The pollinated flower or pod is saturated with a light mist of the hormone mixture, taking care not to cause the flower/pod to prematurely detach from the plant. The mixture contains 100 mg GA3, 25 mg NAA and 5 mg kinetin/L distilled water. These hormones aid in the retention of the developing pod and in increased pod growth.

Pods from wide crosses are harvested at approximately 14 to 16 days post pollination. Before selecting an individual pod to harvest, it is verified that the sepals were removed (to indicate a wide cross attempt) and that the seed size is as expected for a wide cross. Pods are collected and counted according to wide cross combination to determine crossing success. The average crossing success may be approximately 40%. The wide cross pods can contain 1 to 3 seeds but generally 2 seeds are found in each F1 pod.

Harvested pods are collected and brought back to the lab to be sterilized. The pods are first rinsed with 70% EtOH for 2 to 3 minutes and then placed in 10% Clorox bleach for an additional 30 minutes on a platform shaker at approximately 130 RPM. Finally, the pods are rinsed multiple times with sterile water to remove any residual bleach. Embryo isolation can begin immediately following pod sterilization or pods can be stored at 4° C. for up to 24 hours prior to embryo isolation. The sterilized pods are next taken to a laminar flow hood where the embryos can be rescued. Individual pods are placed in a sterile petri dish and opened using a scalpel and forceps. An incision is made along the length of the wide cross pod away from the seed. The pod can then be easily opened to expose the seed. Alternatively, two pair of forceps can be used to separate the pod shell. The seed is carefully removed from the pod and placed in a sterile petri dish under the dissection microscope. Very fine forceps are used to isolate the embryo from the seed. With forceps in one hand, the side of the seed away from the embryo is gently held, with hilum facing up. Using another pair of forceps in the other hand, the seed coat is removed from the side of the seed containing the embryo. The membrane surrounding the embryo is peeled off and the embryo is pushed up from the bottom side. Embryos should be past the globular developmental stage and preferably past the early heart developmental stage (middle to late heart stage, cotyledon stage and early maturation stage embryos are desired). Isolated embryos are transferred to embryo rescue medium. Embryos can be treated to induce chromosome doubling at this time. (See below for chromosome doubling details.) Isolated embryos remain on embryo rescue medium for 21 to 30 days at 24° C. Embryos may remain in the dark for the entire incubation on the embryo rescue medium, may begin the incubation in the dark and complete it in the light, or may spend the entire incubation in the light. There is not a callus induction stage in this protocol. Shoots are developed directly from the embryos.

Either colchicine or trifluralin (both, Sigma-Aldrich, St. Louis, MO) can be used to induce chromosome doubling. Ideally, late heart stage wide cross embryos (or larger) are chemically treated to induce chromosome doubling at any time from immediately following isolation up to 1 week post isolation. The doubling agent can be mixed in either solid or liquid medium and applied for several hours or up to a few days. Trifluralin is used at a concentration of 10-40 uM in either solid or liquid media. Additionally, colchicine is used at a concentration of 0.4-1 mg/ml in either solid or liquid media. Following the chemical treatment, the embryos are transferred to fresh embryo rescue medium.

Developing embryos are transferred from rescue medium to germination medium such as Soy ER GSMv2 (i.e. 3.1 g B5 basal salt, Gamborg's, 1 ml B5 vitamins 1000×, 40 g sucrose [C12H22O11], 0.25 g casein hydrolysate, 0.25 ml BAP, 0.75 g MgCl2*6H20, 20 ml glutamine 25 mg/ml, 0.1 g serine [C3H7NO3], 4 ml Asparagine 25 mg/ml and 0.05 ml of IBA 1 mg/ml) for approximately 3 to 5 weeks in the light at 24° C. Alternatively, developing embryos may be transferred from rescue medium to elongation medium such as Soy E1 0 No TCV (i.e., 4.3 g MS Basal salt Mixture [MSPO1], 5 ml MS iron 200×, 30 g Sucrose [C12H22011], 1 g MES [C6H13NO4S], 8 g purified agar, 1 ml B5 vitamins 100×, 2 ml glutamine 25 mg/ml, 0.50 ml zeatin riboside, trans isomers 1 mg/ml, 0.1 ml IAA 1 mg/ml, 0.2 ml GA3 5 mg/ml, 1.5 ml timentin 100 mg/ml, 0.3 ml cefotaxime 250 mg/ml, 0.5 ml vancomycin 100 mg/ml) for approximately 3 to 5 weeks in the light at 24° C. Developing shoots may be transferred from media plates to Phytocons (PhytoTechnology Laboratories, Lenexa, KS) containing either germination or elongation medium for further shoot development. Established shoots are moved to soil. Initial plant care is critical for survival of these shoots.

Ploidy analysis is conducted using a flow cytometer. Leaf tissue for ploidy analysis is collected from small shoots either in culture or after establishment in soil. Tissue is collected on dry ice and stored at −80° C. until analysis or collected on wet ice and analyzed the same day. A sample size of 0.5 cm² is sufficient. Samples are prepared according to standard techniques. Each sample set contains an untreated F1 plant (not treated to induce chromosome doubling) as a control.

The method described above results in a significantly higher wide cross success rate than is reported in literature. Further, no emasculation of female flowers is required, which saves time and reduces risk of damage to the stigma.

### Example 4: Identification of Candidate R-Genes within the Identified Chromosomal Interval

Further genotyping of the G. tomentella chromosomal interval (SEQ ID NO: 1) led to the discovery of three potential causative genes for ASR resistance (herein also referred to as the candidate R-genes) located on chromosome 3 within the disclosed interval. Associations between each of the candidate genes and ASR resistance was validated and the efficacy of each of the genes in conferring ASR resistance was assessed.

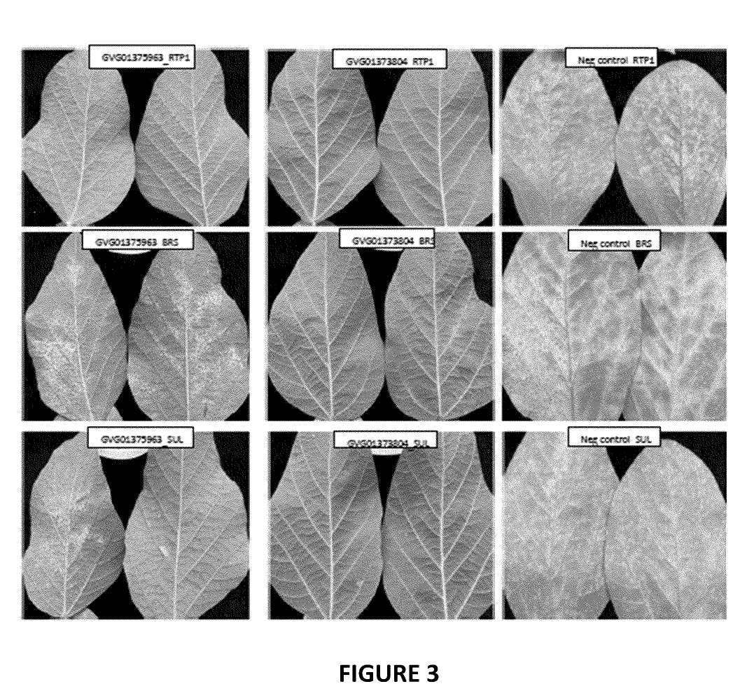

One of the genes, herein also referred to as “GtoRG30”, encodes an R-gene with a TNL motif (SEQ ID NOS: 2-4). The sequence of the polypeptide encoded by the R-gene is depicted at SEQ ID NO: 5. The native promoter for this gene is provided at SEQ ID NO: 15. Details regarding the validation and efficacy of the R-gene is provided at Examples 6, 8 and FIGS. 3-5 and 9-10.

Additionally, two other R genes encoding a CNL motif were identified, as depicted at SEQ ID NOs: 6 and 7. Both these R-genes were validated.

It is contemplated that the R-genes of the present invention, or variants, fragments, homologs or orthologs thereof, can be employed in a transgenic, gene editing, or breeding method utilizing embryo rescue, tetraploid soy, or other introgression methods, as described above, to generate plants having increased resistance to fungal pathogens including ASR. Additionally, or optionally, nucleic acid molecules comprising the R-gene coding sequence of the present invention (e.g., any of SEQ ID NOS: 2-4, 11-12), or nucleic acid molecules with a nucleotide sequence substantially identical to the R-gene coding sequence of the present invention (e.g., SEQ ID NOs: 2-4, 11-12), or nucleic acid molecules comprising a nucleotide sequence encoding a polypeptide having the amino acid sequence of SEQ ID NO: 5, or a polypeptide having an amino acid sequence substantially identical to SEQ ID NO: 5, can be employed in a transgenic, gene editing, or breeding method utilizing embryo rescue, tetraploid soy, or other introgression methods, as described above to generate pathogen resistant (e.g., ASR resistant) plants.

Given the sequence of the R-gene and the associated trait (e.g. ASR resistance), oligonucleotide primers can be developed and use said primers to identify plants carrying the gene with the nucleotide sequence depicted in SEQ ID NOs: 2-4, 11-12. A TAQMAN® assay (e.g. generally a two-step allelic discrimination assay or similar), a KASP™ assay (generally a one-step allelic discrimination assay defined below or similar), or both can be employed to assay the genes. In an exemplary two-step assay, a primer pair comprising a forward primer and a reverse primer are employed to amplify the gene, or a functional part thereof, associated with conferring ASR resistance. Further, an assay probe (or hybridization oligo) can be employed with the primer pair to detect a target sequence present in the amplified gene. The probe may be labeled with, for example, fluorophores to permit easy detection. Further, the probes may include a minor groove binder (MGB) moiety at the 3′ end that increases the melting temperature (Tm) of the probe and stabilizes probe-target hybrids. This allows the length of the probe to be shortened while still providing sequence discrimination and flexibility to accommodate the target. Further, the probe can include a nonfluorescent quencher (NFQ) to absorb (quench) signal from the fluorescent dye label at the other end of the probe, reducing background noise and improving sensitivity of the probe. In some embodiments, the assay primers and the reaction conditions are designed such that an assay primer will only hybridize to the reverse complement of a 100% perfectly matched sequence, thereby permitting detection of the gene based upon detection of hybridizations.

As a non-limiting example, presence of a nucleic acid molecule comprising the R-gene sequence of any of SEQ ID NOs: 2-4, 11-12 or a sequence having at least 90% sequence identity, at least 95% sequence identity, or at least 99% sequence identity to any of SEQ ID NO: 2-4, 11-12; or a nucleotide sequence encoding the protein of SEQ ID NO: 5 or encoding a protein having at least 90% sequence identity, at least 95% sequence identity, or at least 99% sequence identity to SEQ ID NO: 5, can be detected by generating an amplicon using the primer pair of SEQ ID NOs: 8-9 and/or detected using the probe sequence of SEQ ID NO: 10, the probe comprising a FAM fluorophore at the 5′-end and an MGB and NFQ moiety at the 3′-end. In one example, presence of the R-gene, or a functional part thereof, in a disease resistant plant (e.g., ASR resistant soybean plant) may be identified by isolating nucleic acid molecules from said plant and generating an amplicon comprising at least a portion of the R-gene using the above-mentioned primers and/or probes.

In still further examples, presence of a nucleic acid molecule comprising the R-gene sequence of any of SEQ ID NOs: 2-4, 11-12 or a sequence having at least 90% sequence identity, at least 95% sequence identity, or at least 99% sequence identity to any of SEQ ID NO: 2-4, 11-12 or a nucleotide sequence encoding the protein of SEQ ID NO: 5 or encoding a protein having at least 90% sequence identity, at least 95% sequence identity, or at least 99% sequence identity to SEQ ID NO: 5, can be determined by detecting for the presence of the R-protein encoded by the R-gene. For example, presence of the R-gene, or a functional part thereof, in a disease resistant plant (e.g., ASR resistant soybean plant) can be determined by isolating proteins from said plant and detecting the presence of a protein encoded by the R-gene (such as a protein having the polypeptide sequence of SEQ ID NO: 5, or at least 90% sequence identity to SEQ ID NO: 5) using commonly known protein detection assays (e.g., Western blot, ELISA, radioimmunoassay, etc.).

In still further examples, presence of a nucleic acid molecule comprising the R-gene sequence of any of SEQ ID NOs: 2-4, 11-12 or a sequence having at least 90% sequence identity, at least 95% sequence identity, or at least 99% sequence identity to any of SEQ ID NO: 2-4, 11-12; or a nucleotide sequence encoding the protein of SEQ ID NO: 5 or encoding a protein having at least 90% sequence identity, at least 95% sequence identity, or at least 99% sequence identity to SEQ ID NO: 5, can be detected by generating an amplicon using a primer pair comprising a forward primer and a reverse primer selected from the primers of Tables 5-6, and detected using a probe selected from the probes of Tables 5-6.

### Example 5: Construction of Vectors comprising R-Genes and Transformation

DNA constructs, particularly vectors, were generated comprising the R-gene operably coupled to a heterologous promoter. The nucleotide sequence of the R-gene used in the vectors comprised genomic DNA sequences or coding sequences for the R-gene, herein also referred to as “GtoRG30” and previously described in Example 4 (SEQ ID NOS: 2-4, 11-12). The DNA constructs comprise the R-gene coding sequence operably linked to a heterologous promoter capable of enabling expression of the R-gene in a plant cell. In a first set of constructs, transcription of R-gene GtoRG30 was driven by Medicago truncatula promoter prMt12344. In a second set of constructs, transcription of R-gene GtoRG30 was driven by Medicago truncatula promoter prMt51186. In a third set of constructs, transcription of R-gene GtoRG30 was driven by a native promoter (RG30_promoter). Binary vectors created comprising R-gene GtoRG30 are listed in Table 7.

TABLE 7
Constructs created using the identified novel R-gene

| | | | R-gene sequence |
|---|---|---|---|
| Construct ID | Promoter used | Terminator used | used |
| 25845 | prMt12344 | tMt12344 | gGtoRG30-02 |
| | (SEQ ID NO: 13) | (SEQ ID NO: 16) | (SEQ ID NO: 3) |
| 25899 | prMt51186 | prMt51186 | gGtoRG30-02 |
| | (SEQ ID NO: 14) | (SEQ ID NO: 17) | (SEQ ID NO: 3) |
| 25992 | prMt12344 | tMt12344 | cGtoRG30-01 |
| | (SEQ ID NO: 13) | (SEQ ID NO: 16) | (SEQ ID NO: 4) |
| 26015 | prMt51186 | tMt51186 | cGtoRG30-01 |
| | (SEQ ID NO: 14) | (SEQ ID NO: 17) | (SEQ ID NO: 4) |
| 25950 | Native | Native | gGtoRG30-02 |
| | RG30_promoter | RG30_terminator | (SEQ ID NO: 11) |
| | (SEQ ID NO: 15) | (SEQ ID NO: 18) | |

a) 25845 Vector Construction for R-Gene Comprising SEQ ID NO: 3

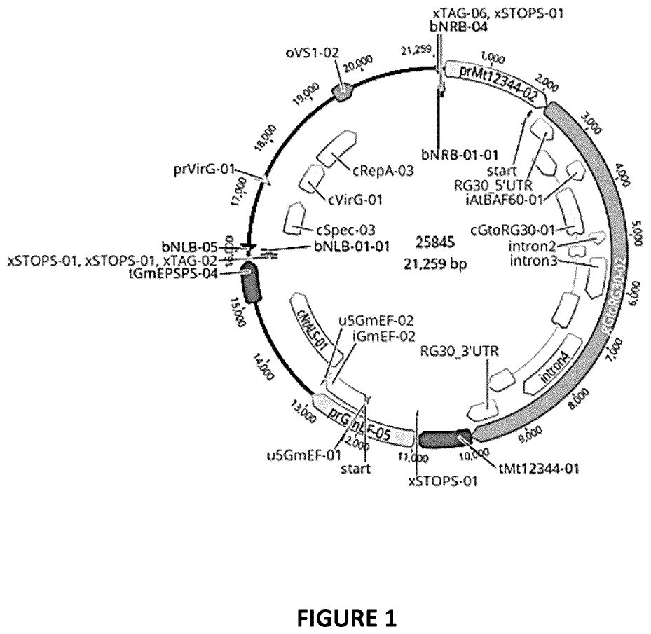

FIG. 1 provides an illustration of vector 25845 for an R-gene comprising SEQ ID NO: 3. Features are described below.
Vector type: Binary Vector
Construct Size (bp): 30,982

Functional description: A binary vector for soybean transformation with the ALS selection harboring a soy rust resistance candidate gene, gGtoRG30-02 from chr3b of PI_505267 (G. tomentella), encoding a protein containing toll/interleukin receptor-1 (TIR), nucleotide-binding site (NBS), and leucine rich repeat (LRR) domains. This resistance gene includes its native 5′UTR and 3′UTR and the coding sequence cGtoRG30-01. The first native intron was replaced with Arabidopsis intron. This resistance gene is driven by the Medicago truncatula promoter, prMt12344-02 and corresponding terminator, tMt12344-01. Vector also contains the ALS selection cassette prGmEF-05/cNtALS-01/tGmEPSPS-04.

Features:

oVS1. Start: 19359 End: 19763. Origin of replication in Agrobacterium tumefaciens host.

cRepA. Start: 18243 End: 19316. cRepA-01 with A to G at nt735.

cVirG. Start: 17488 End: 18213. virG (putative) from pAD1289 with TTG start codon. virGN54D came from pAD1289 described in Hansen et al. 1994, PNAS 91:7603-7607.

prVirG. Start: 17283 End: 17413. virG promoter (Winans J. Bact. 172:2433-38 (1990)) composed of two promoter elements, one responsive to acetosyringone and phosphate-starvation (bp 45 to 83) and another to medium acidification (86 to 128).

cSpec. Start: 16400 End: 17188. Also called aadA; gene encoding the enzyme aminoglycoside 3′adenyltransferase that confers resistance to spectinomycin and streptomycin for maintenance of the vector in E. coli and Agrobacterium.

bNLB. Start: 16026 End: 16050. 25 bp Left border repeat region of T-DNA of Agrobacterium tumefaciens nopaline ti-plasmid.

bNLB. Start: 15991 End: 16120. Left border region of T-DNA of Agrobacterium tumefaciens nopaline ti-plasmid.

xTAG. Start: 15943 End: 15982. 40 bp site for plant insert intactness testing and to stop readthrough ORFs.

xSTOPS Start: 15931 End: 15942. 6-frame stop to minimize unintended ORF read-through.

xSTOPS Start: 15855 End: 15866. 6-frame stop to minimize unintended ORF read-through.

tGmEPSPS Start: 15057 End: 15854. An EPSPS terminator from Glycine max.

cNtALS Start: 13056 End: 15050. The NtALS DNA fragment encodes an Acetolactate synthase (ALS) double mutant (P191A, W568L) from Nicotiana tabacum. It was codon-optimized for soybean expression.

u5GmEF Start: 13037 End: 13047. Second 5′ UTR of the soybean elongation factor (EF) gene.

iGmEFStart: 12128 End: 13036. The first intron of the soybean elongation factor (EF) gene. iGmEF-01 with an internal BamHI site and a 3′ end unintended ORF removed.

u5GmEF Start: 12065 End: 12127. First 5′ UTR of the soybean elongation factor (EF) gene.

start Start: 12065 End: 12065. Transcription start site.

prGmEF Start: 10982 End: 13047. Translation elongation factor EF-1 alpha/Tu promoter, including the first intron and neighboring UTR, from soybean (williams 82).

xSTOPS Start: 10970 End: 10981. 6-frame stop to minimize unintended ORF read-through.

tMt12344 Start: 9956 End: 10962. The terminator based on the Medicago truncatula gene. It consists of the 3′-UTR and 3′-non-transcribed sequence.

gGtoRG30-02 Start: 2231 End: 9955. A genomic fragment containing a soy R-gene (SEQ ID NO: 3) along with its native 5′UTR and 3′UTR that encodes a protein containing toll/interleukin receptor-1 (TIR), nucleotide-binding site (NBS), and leucine rich repeat (LRR) domains. The first native intron was replaced with the Arabidopsis intron, iAtBAF60-01. The genomic fragment comprises the following components: RG30_5′UTR Start: 2231; End: 2730; RG30_3′UTR Start: 9318 End: 9955; intron4 Start: 7094 End: 8693; intron3 Start: 5401 End: 6262; intron2 Start: 4877 End: 5124; iAtBAF60-01 Start: 3357 End: 3765; Intron of Arabidopsis thaliana BAF60 homolog (CHC1 by the Chromatin database) inserted in GUS coding sequence to prevent bacterial expression; cGtoRG30-01 Start: 2731 End: 9317; the CDS from the R-gene. The coding sequence comprises (with reference to SEQ ID NO: 3):

| | | |
|---|---|---|
| gGtoRG30-02 | 1 | 7,725 |
| RG30_3′UTR | 7,088 | 7,725 |
| intron4 | 4,864 | 6,463 |
| intron3 | 3,171 | 4,032 |
| intron2 | 2,647 | 2,894 |
| iAtBAF60-01 | 1,127 | 1,535 |
| cGtoRG30-01 (CDS) | 501 | 7,087 |
| RG30_5′UTR | 1 | 500 |

start Start: 2043 End: 2043 The transcription start site based on cDNA/gDNA alignment.

prMt12344 Start: 217 End: 2218 The promoter from the Medicago truncatula gene. It consists of 5′-non-transcribed sequence and the 5′ UTR.

xSTOPS Start: 184 End: 195 6-frame stop to minimize unintended ORF read-through

xTAG Start: 144 End: 183 40 bp site for plant insert intactness testing and to stop readthrough ORFs.

bNRB-01 Start: 101 End: 125 Right Border Repeat

bNRB-04 Start: 4 End: 143 Right border region of T-DNA of Agrobacterium tumefaciens nopaline ti-plasmid.

b) 25899 Vector Construction for R-Gene Comprising SEQ ID NO: 3

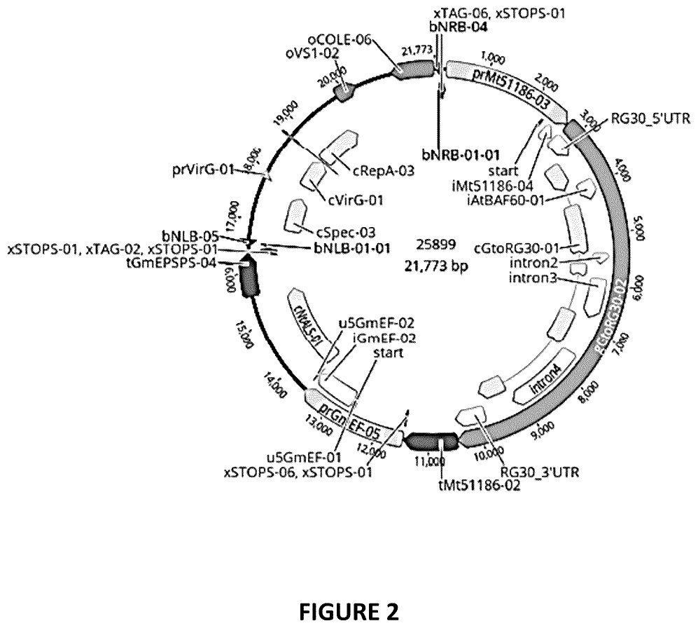

FIG. 2 provides an illustration of vector 25899 for an R-gene comprising SEQ ID NO: 4. Features are described below.

oCOLE Start: 20,955 End: 21,761 The ColE1 origin of replication functional in E. coli derived from pUC19.

oVS1 Start: 19,873 End: origin of replication in Agrobacterium tumefaciens host.

cRepA Start: 18,757 End: 19,830 cRepA with A to G at nt735.

cVirG Start: 18,002 End: 18,727 virG (putative) from pAD1289 with TTG start codon. virGN54D came from pAD1289 described in Hansen et al. 1994, PNAS 91:7603-7607.

prVirGStart: 17,797 End: 17,927 virG promoter (Winans J. Bact. 172:2433-38 (1990)) composed of two promoter elements, one responsive to acetosyringone and phosphate-starvation (bp 45 to 83) and another to medium acidification (86 to 128).

cSpec Start: 16,914 End: 17,702 Also called aadA; gene encoding the enzyme aminoglycoside 3′adenyltransferase that confers resistance to spectinomycin and streptomycin for maintenance of the vector in E. coli and Agrobacterium.

bNLB Start: 16,540 End: 16,564 25 bp Left border repeat region of T-DNA of Agrobacterium tumefaciens nopaline ti-plasmid.

bNLB Start: 16,505 End: 16,634 Left border region of T-DNA of Agrobacterium tumefaciens nopaline ti-plasmid.

xTAG Start: 16,457 End: 16,496 40 bp site for plant insert intactness testing and to stop readthrough ORFs.

xSTOPS Start: 16,445 End: 16,456 6-frame stop to minimize unintended ORF read-through.

xSTOPS Start: 16,369 End: 16,380 6-frame stop to minimize unintended ORF read-through.

tGmEPSPS Start: 15,571 End: 16,368 An EPSPS terminator from Glycine max. Removal of 6 frame stops at 3′ end of terminator.

cNtALS Start: 13,570 End: 15,564 The NtALS DNA fragment encodes an Acetolactate synthase (ALS) double mutant (P191A, W568L) from Nicotiana tabacum. It was codon-optimized for soybean expression and synthesized by GeneArt.

u5GmEF Start: 13,551 End: 13,561 Second 5′ UTR of the soybean elongation factor (EF) gene.

iGmEF Start: 12,642 End: 13,550 The first intron of the soybean elongation factor (EF) gene.

u5GmEF Start: 12,579 End: 12,641 First 5′ UTR of the soybean elongation factor (EF) gene.

start Start: 12,579 End: 12,579 transcription start site.

prGmEF Start: 11,496 End: 13,561 Translation elongation factor EF-1 alpha/Tu promoter, including the first intron and neighboring UTR, from soybean (williams 82).

xSTOPS Start: 11,484 End: 11,495 6-frame stop to minimize unintended ORF read-through.

xSTOPS Start: 11,465 End: 11,476 6-frame stop to minimize unintended ORF read-through.

tMt51186 Start: 10,465 End: 11,464 Modified terminator of Medicago truncatula gene.

gGtoRG30-02 (SEQ ID NO: 3) Start: 2,734 End: 10,458; A genomic fragment containing a soy R-gene along with its native 5′UTR and 3′UTR that encodes a protein containing toll/interleukin receptor-1 (TIR), nucleotide-binding site (NBS), and leucine rich repeat (LRR) domains. The first native intron was replaced with the Arabidopsis intron, iAtBAF60-01. The genomic fragment comprises the following components: RG30_5′UTR Start: 2,734 End: 3,233; RG30_3′UTR Start: 9,821 End: 10,458; intron4 Start: 7,597 End: 9,196; intron3 Start: 5,904 End: 6,765; intron2 Start: 5,380 End: 5,627; iAtBAF60-01 Start: 3,860 End: 4,268; intron of Arabidopsis thaliana BAF60 homolog (CHC1 by the Chromatin database) inserted in GUS coding sequence to prevent bacterial expression; cGtoRG30-01 Start: 3,234 End: 9,820; the CDS of the R-gene.

iMt51186 Start: 2,450 End: 2,700 The first intron of the Medicago truncatula gene.

start Start: 2,313 End: 2,313 Transcription start based on cDNA/gDNA alignment.

prMt51186 Start: 217 End: 2,721 The promoter from the Medicago truncatula gene.

xSTOPS Start: 184 End: 195 6-frame stop to minimize unintended ORF read-through.

xTAG Start: 144 End: 183 40 bp site for plant insert intactness testing and to stop readthrough ORFs.

bNRB Start: 101 End: 125 Right Border Repeat

bNRB-04 Start: 4 End: 143 Right border region of T-DNA of Agrobacterium tumefaciens nopaline ti-plasmid. Differs from bNRB-03 by 20 bp at 5′ end.

c) 25950 Vector Construction for R-Gene Comprising SEQ ID NO: 11

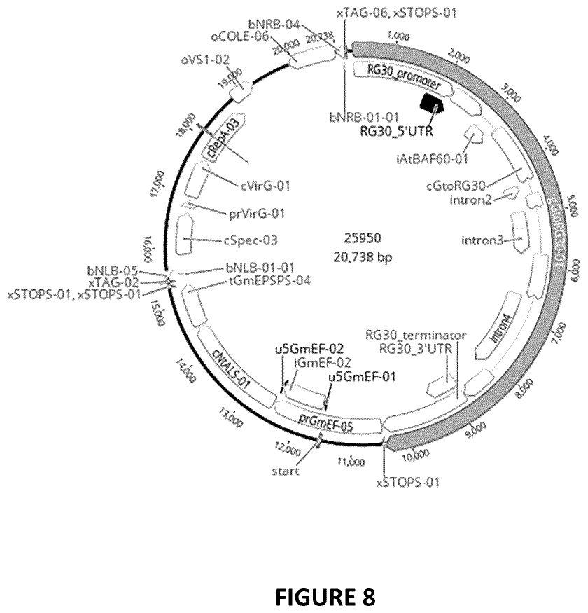

FIG. 8 provides an illustration of vector 29590 for an R-gene comprising SEQ ID NO: 11. Features are described below.

Vector type: Binary Vector

Construct Size (bp): 20,738

Functional description: A binary vector for soybean transformation with the ALS selection harboring a soy rust resistance candidate gene, gGtoRG30-01 encoding a protein containing toll/interleukin receptor-1 (TIR), nucleotide-binding site (NBS), and leucine rich repeat (LRR) domains. The first native intron was replaced with Arabidopsis intron. Vector also contains the ALS selection cassette prGmEF-05/cNtALS-01/tGmEPSPS-04.

Features:

xTAG Start: 144 End: 183 40 bp site for plant insert intactness testing and to stop readthrough ORFs.

xTAG Start: 15,422 End: 15,461 40 bp site for plant insert intactness testing and to stop readthrough ORFs.

xSTOPS Start: 15,410 End: 15,421 6-frame stop to minimize unintended ORF read-through

xSTOPS Start: 15,334 End: 15,345 6-frame stop to minimize unintended ORF read-through

xSTOPS Start: 10,449 End: 10,460 6-frame stop to minimize unintended ORF read-through

xSTOPS Start: 184 End: 195 6-frame stop to minimize unintended ORF read-through

prGmEF Start: 12,516 End: 12,526 Translation elongation factor EF-1 alpha/Tu promoter, including the first intron and neighboring UTR, from soybean (williams 82).

prGmEF Start: 11,544 End: 11,606 Translation elongation factor EF-1 alpha/Tu promoter, including the first intron and neighboring UTR, from soybean (williams 82).

tGmEPSPS Start: 15,536 End: 15,333 An EPSPS terminator from Glycine max.

gGtoRG30-01 Start: 8,804 End: 10,441 A genomic fragment containing a soy rust resistance candidate gene that encodes a protein containing toll/interleukin receptor-1 (TIR), nucleotide-binding site (NBS), and leucine rich repeat (LRR) domains. The first native intron was replaced with Arabidopsis intron, iAtBAF60-01. The coding sequence comprises (with reference to SEQ ID NO: 11):

| | | |
|---|---|---|
| gGtoRG30-01 loci | 1 | 10,158 |
| RG30_3′UTR | 8,521 | 9,158 |
| RG30_terminator | 8,521 | 10,158 |
| intron4 | 6,297 | 7,896 |
| intron3 | 4,604 | 5,465 |
| intron2 | 4,080 | 4,327 |
| iAtBAF60-01 | 2,560 | 2,968 |
| cGtoRG30 (CDS) | 1,934 | 8,520 |
| RG30_5′UTR | 1,434 | 1,933 |
| RG30_promoter | 1 | 1,933 |

prVirG Start: 16,762 End: 16,892 virG promoter (Winans J. Bact. 172:2433-38 (1990)) composed of two promoter elements, one responsive to acetosyringone and phosphate-starvation (bp 45 to 83) and another to medium acidification (86 to 128)

prGmEF Start: 10,461 End: 12,526 Translation elongation factor EF-1 alpha/Tu promoter, including the first intron and neighboring UTR, from soybean (williams 82).

oVS1 Start: 18,838 End: 19,242 origin of replication in Agrobacterium tumefaciens host

oCOLE Start: 20,726 End: 19,920 The ColE1 origin of replication functional in E. coli

prGmEF-05 Start: 11,607 End: 12,515 Translation elongation factor EF-1 alpha/Tu promoter, including the first intron and neighboring UTR, from soybean (williams 82).

iAtBAF60 Start: 2,843 End: 3,251 intron of Arabidopsis thaliana BAF60 homolog (CHC1 by the Chromatin database) inserted to prevent bacterial expression</nobr></html>

cVirG Start: 16,967 End: 17,692 virG from pAD1289 with TTG start codon described in Hansen et al. 1994, PNAS 91:7603-7607

cSpec Start: 15,879 End: 16,667 gene encoding the enzyme aminoglycoside 3′adenyltransferase that confers resistance to spectinomycin and streptomycin for maintenance of the vector in E. coli and Agrobacterium.

cRepA Start: 17,722 End: 18,795 cRepA-01 with A to G at nt735

cNtALS Start: 12,535 End: 14,529 encodes an Acetolactate synthase double mutant from Nicotiana tabacum that was codon-optimized for soybean expression

bNRB Start: 125 End: 101 Right border region of T-DNA of Agrobacterium tumefaciens nopaline ti-plasmid

bNLB Start: 15,529 End: 15,505 Left border repeat region of T-DNA of Agrobacterium tumefaciens nopaline ti-plasmid

Each of the constructs was transformed into soybean cells using known methods of plant transformation (e.g., via Agrobacterium mediated transformation) to create primary soybean events.

### Example 6: Validation of R-Genes Against ASR

In the phenotyping work, both symptom evaluation and molecular assays for rust resistance or susceptibility ratings have been used. The symptom evaluation is a modified version of a rust rating scale from Burdon and Speer (Euphytica, 33: 891-896, 1984; also T A G 1984). The molecular assay is based on a fungal housekeeping gene, β-tubulin, wherein the probe for β-tubulin targets a specific region in soy rust but not in other pathogens or plant species. Further, the molecular assay was validated by coupling with phenotypic symptomatic observations, as shown in FIGS. 3-4 and 9.

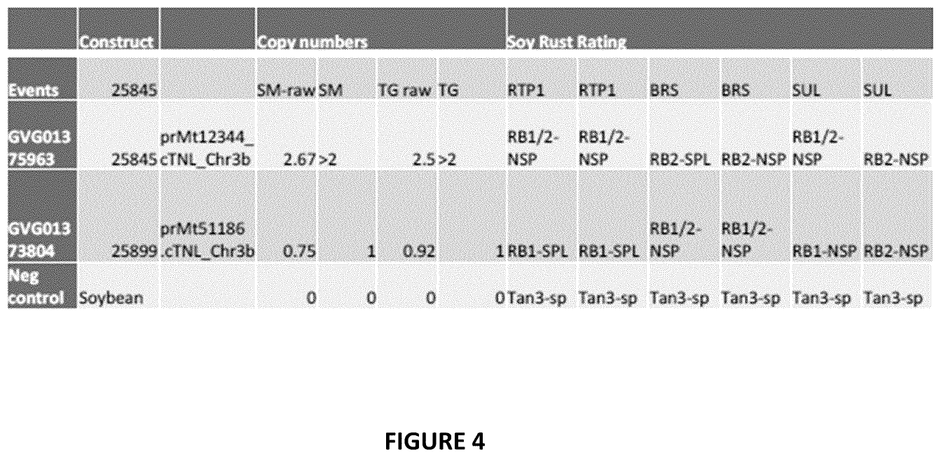

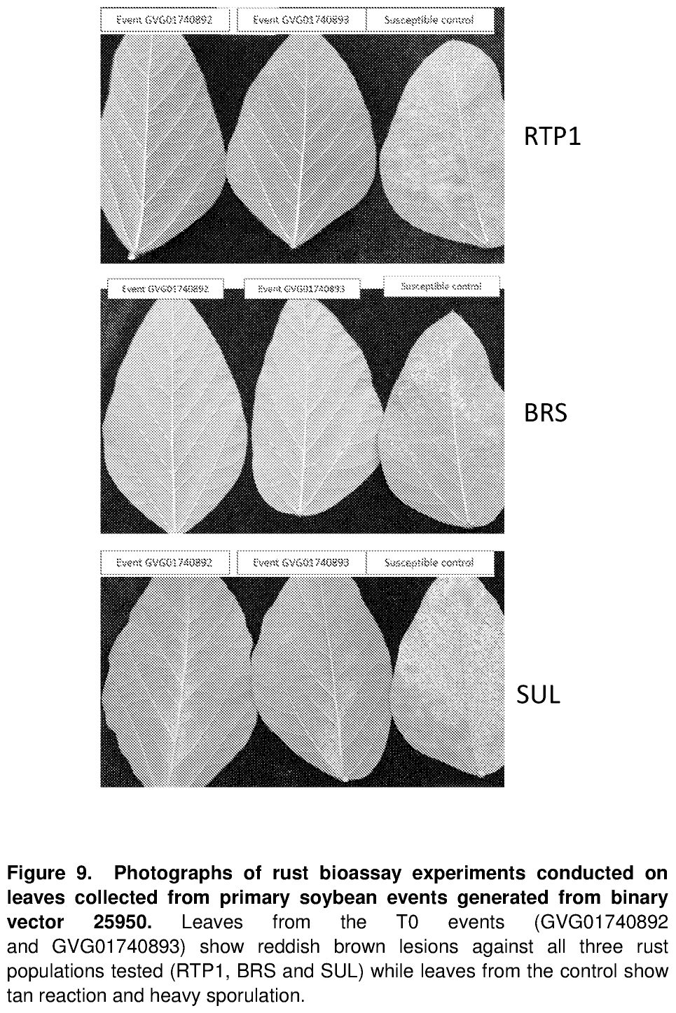

FIG. 3 compares leaves from primary soybean events generated from transformation of binary construct 24845 (TO event GVG01375963) and binary construct 25899 (TO event GVG013773804) with leaves from a control. FIG. 9 compares leaves from primary soybean events generated from binary construct 25950 (TO events GVG01740892 and GVG01740893) with leaves from a control. FIG. 4 shows the Disease resistance ratings of the primary soybean events relative to the control. The control has the same genetic background without the transgene.

Leaves from primary events were placed in a petri dish on moist paper towel and then inoculated with a soybean rust spore suspension from three different rust populations (RTP1; BRO1—Brazil; BRO3—Brazil). Leaves from plants that had the same genetic background but did not have the transgene served as negative controls. After 14 days, leaves from both events and the control were evaluated for resistance to soybean rust. As shown in FIGS. 3, 4 and 9, the leaves from T0 events show clear evidence of resistance to soybean rust compared to the wild type control. The T0 event leaves show the presence of reddish-brown lesions (first two panels on each row) while leaves from the control (last panel on each row) show tan reaction and are heavily sporulating, thus indicating that the novel R-gene confers resistance to ASR.

The results in FIGS. 3 and 9 are shown using the standard soy rust rating scale with RB types indicative of the event being resistant and Tan ratings indicative of the event being susceptible. Numbers listed after the RB are a rating of combination of density of lesions or size of the lesions with 1-4 scale from high to intermediate resistance, with no sporulation (NSP) or very little sporulation (SPL). Numbers listed after Tan is a rating of combination of density of pustules and level of sporulation with 1-5 scale from low to high sporulation levels. As can be seen from the difference in ratings, the TO events repeatedly shows high level of disease resistance compared to the control.

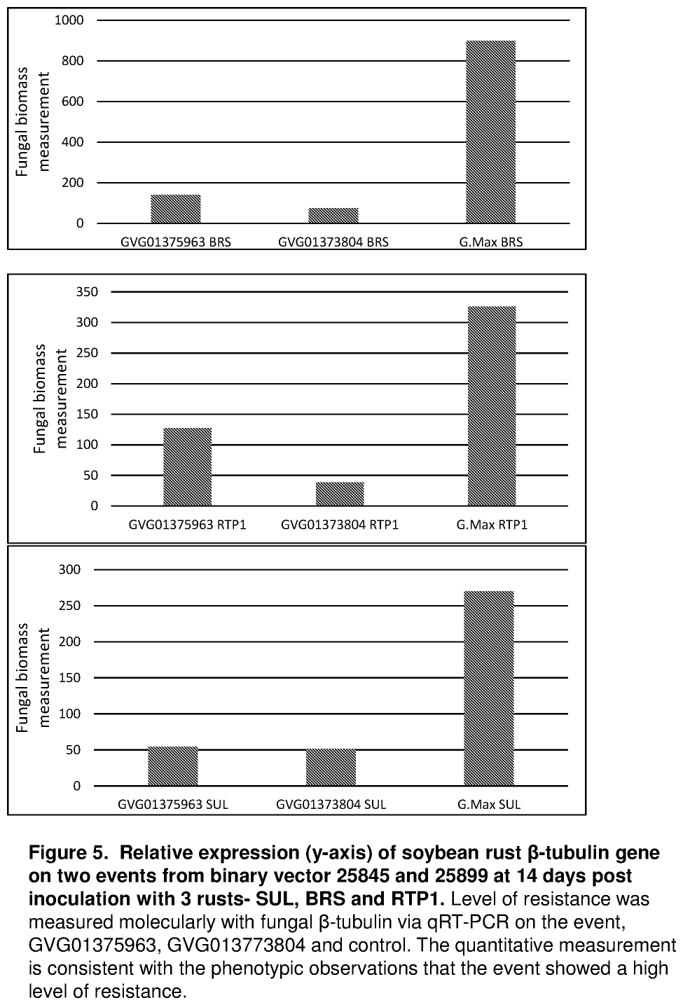

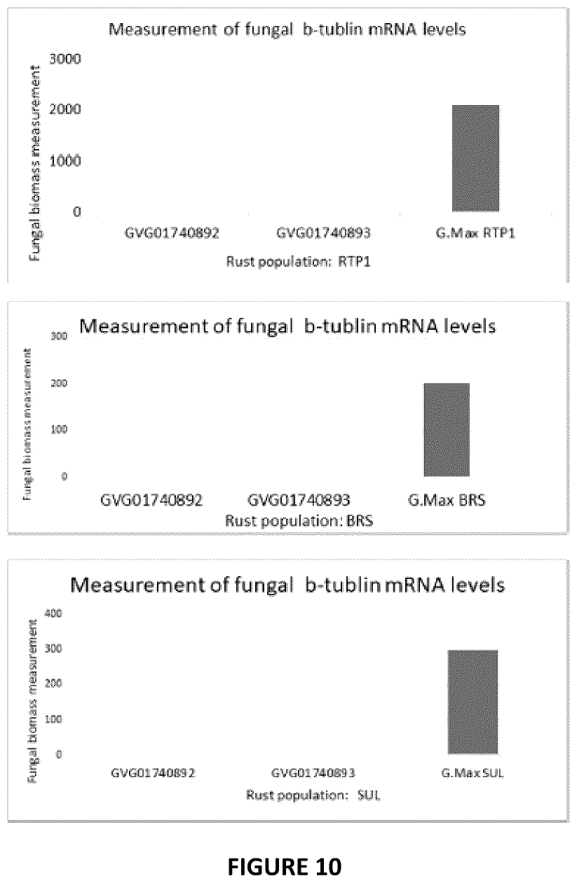

Quantitative measurements taken using fungal β-tubulin transcripts are consistent with these phenotypic observations, as shown in the graph of FIGS. 5 and 10. The level of resistance was measured molecularly with fungal β-tubulin via qRT-PCR on the event and control. The event comprising the TNL R-gene showed a high level of resistance with more than 90% reduction in fungal biomass as compared to the control. The average qRT value for the control is about 981, while the average qRT value for the event is about 79. The data also shows that the identified TNL R-gene is expressed in the assayed events.

In this validation, we demonstrated that construct 25845 and construct 25950 show strong resistance (>90%) and broad spectrum against all rusts tested.

### Example 7: Additional Vector Construction Examples

Additional vector constructs are also provided by way of example.

d) 25992 Vector Construction for R-Gene Comprising SEQ ID NO: 4

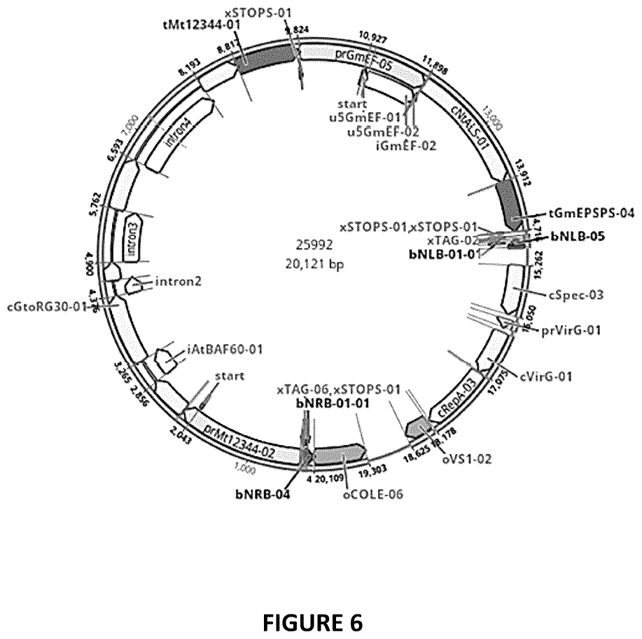

FIG. 6 provides an illustration of vector 25992 for an R-gene comprising SEQ ID NO: 4. Features are described below.

oCOLE Start: 19,303 End: 20,109. The ColE1 origin of replication functional in E. coli.

oVS1 Start: 18,221 End: 18,625. Origin of replication in Agrobacterium tumefaciens host.

cRepA Start: 17,105 End: 18,178. cRepA-01 with A to G at nt735.

cVirG Start: 16,350 End: 17,075. virG (putative) from pAD1289 with TTG start codon. virGN54D came from pAD1289 described in Hansen et al. 1994, PNAS 91:7603-7607.

prVirG Start: 16,145 End: 16,275. virG promoter (Winans J. Bact. 172:2433-38 (1990)) composed of two promoter elements, one responsive to acetosyringone and phosphate-starvation (bp 45 to 83) and another to medium acidification (86 to 128).

cSpec Start: 15,262 End: 16,050. Also called aadA; gene encoding the enzyme aminoglycoside 3′adenyltransferase that confers resistance to spectinomycin and streptomycin for maintenance of the vector in E. coli and Agrobacterium.

bNLB Start: 14,888 End: 14,912 25 bp. Left border repeat region of T-DNA of Agrobacterium tumefaciens nopaline ti-plasmid.

bNLB Start: 14,853 End: 14,982. Left border region of T-DNA of Agrobacterium tumefaciens nopaline ti-plasmid.

xTAG Start: 14,805 End: 14,844. 40 bp site for plant insert intactness testing and to stop readthrough ORFs.

xSTOPS Start: 14,793 End: 14,804. 6-frame stop to minimize unintended ORF read-through.

xSTOPS Start: 14,717 End: 14,728. 6-frame stop to minimize unintended ORF read-through.

tGmEPSPS Start: 13,919 End: 14,716. Modified version of tGMEPS-02; an EPSPS terminator from Glycine max.

cNtALS Start: 11,918 End: 13,912. The NtALS DNA fragment encodes an Acetolactate synthase double mutant (P191A, W568L) from Nicotiana tabacum. It was codon-optimized for soybean expression.

u5GmEF Start: 11,899 End: 11,909. Second 5′ UTR of the soybean elongation factor (EF) gene.

iGmEFStart: 10,990 End: 11,898. The first intron of the soybean elongation factor (EF) gene.

u5GmEF Start: 10,927 End: 10,989. First 5′ UTR of the soybean elongation factor (EF) gene.

start Start: 10,927 End: 10,927. Transcription start site

prGmEF Start: 9,844 End: 11,909. Translation elongation factor EF-1 alpha/Tu promoter, including the first intron and neighboring UTR, from soybean (williams 82).

xSTOPS Start: 9,832 End: 9,843. 6-frame stop to minimize unintended ORF read-through.

tMt12344 Start: 8,818 End: 9,824. The terminator based on the Medicago truncatula gene.

It consists of the 3′-UTR and 3′-non-transcribed sequence.

| | |
|---|---|
| intron4 Start: 6,594 | End: 8,193 |
| intron3 Start: 4,901 | End: 5,762 |
| intron2 Start: 4,377 | End: 4,624 |

iAtBAF60 Start: 2,857 End: 3,265. Intron of Arabidopsis thaliana BAF60 homolog inserted in GUS coding sequence to prevent bacterial expression.

cGtoRG30 (SEQ ID NO: 4) Start: 2,231 End: 8,817. A CDS of soy R-gene that encodes a protein containing toll/interleukin receptor-1 (TIR), nucleotide-binding site (NBS), and leucine rich repeat (LRR) domains. This CDS is from an R-gene on Chromosome 3 of G. tomentella P1 505267.

start Start: 2,043 End: 2,043. The transcription start site based on cDNA/gDNA alignment.

prMt12344 Start: 217 End: 2,218. The promoter from the Medicago truncatula gene. It consists of 5′-non-transcribed sequence and the 5′ UTR.

xSTOPS Start: 184 End: 195. 6-frame stop to minimize unintended ORF read-through.

xTAG Start: 144 End: 183. 40 bp site for plant insert intactness testing and to stop readthrough ORFs. Typically, by agro RB. 1 bp different than −01.

bNRB Start: 101 End: 125. Right Border Repeat.

bNRB Start: 4 End: 143. Right border region of T-DNA of Agrobacterium tumefaciens nopaline ti-plasmid.

e) 26015 Vector Construction for R-Gene Comprising SEQ ID NO: 4

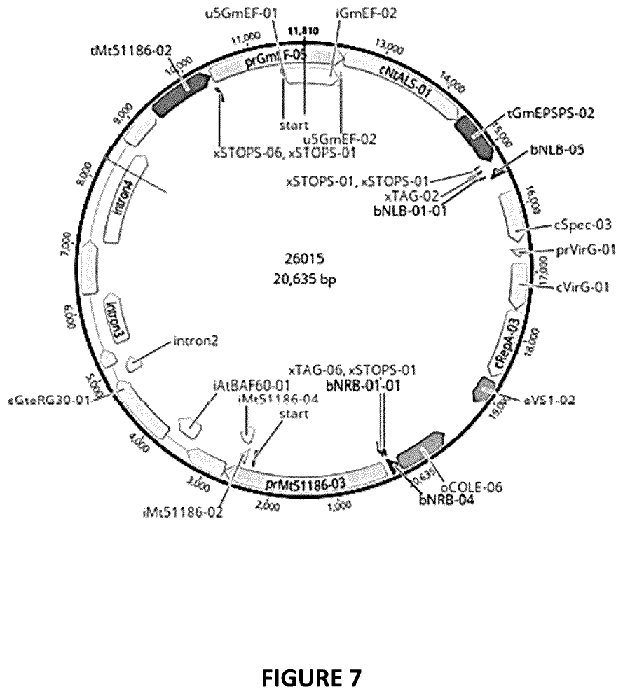

FIG. 7 provides an illustration of vector 26015 for an R-gene comprising SEQ ID NO: 4. Features are described below.

oCOLE Start: 19,817 End: 20,623. The ColE1 origin of replication functional in E. coli.

oVS1 Start: 18,735 End: 19,139. Origin of replication in Agrobacterium tumefaciens host.

cRepA Start: 17,619 End: 18,692. cRepA-01 with A to G at nt735.

cVirG Start: 16,864 End: 17,589. virG (putative) from pAD1289 with TTG start codon. virGN54D came from pAD1289 described in Hansen et al. 1994, PNAS 91:7603-7607.

prVirGStart: 16,659 End: 16,789. virG promoter (Winans J. Bact. 172:2433-38 (1990)) composed of two promoter elements, one responsive to acetosyringone and phosphate-starvation (bp 45 to 83) and another to medium acidification (86 to 128).

cSpec Start: 15,776 End: 16,564. Also called aadA; gene encoding the enzyme aminoglycoside 3′adenyltransferase that confers resistance to spectinomycin and streptomycin for maintenance of the vector in E. coli and Agrobacterium.

bNLB Start: 15,402 End: 15,426 25 bp Left border repeat region of T-DNA of Agrobacterium tumefaciens nopaline ti-plasmid

bNLB Start: 15,367 End: 15,496 Left border region of T-DNA of Agrobacterium tumefaciens nopaline ti-plasmid.

xTAG Start: 15,319 End: 15,358. 40 bp site for plant insert intactness testing and to stop readthrough ORFs.

xSTOPS-01 Start: 15,307 End: 15,318. 6-frame stop to minimize unintended ORF read-through.

xSTOPS Start: 15,231 End: 15,242. 6-frame stop to minimize unintended ORF read-through

tGmEPSPS Start: 14,433 End: 15,242. EPSPS terminator from Glycine max.

cNtALS Start: 12,432 End: 14,426. The NtALS DNA fragment encodes an Acetolactate synthase double mutant (P191A, W568L) from Nicotiana tabacum. It was codon-optimized for soybean expression.

u5GmEF Start: 12,413 End: 12,423. Second 5′ UTR of the soybean elongation factor (EF) gene.

iGmEFStart: 11,504 End: 12,412. The first intron of the soybean elongation factor (EF) gene.

u5GmEF Start: 11,441 End: 11,503. First 5′ UTR of the soybean elongation factor (EF) gene.

start Start: 11,441 End: 11,441. Transcription start site

prGmEF Start: 10,358 End: 12,423. Translation elongation factor EF-1 alpha/Tu promoter, including the first intron and neighboring UTR, from soybean (williams 82).

xSTOPS Start: 10,346 End: 10,357. 6-frame stop to minimize unintended ORF read-through.

xSTOPS Start: 10,327 End: 10,338. 6-frame stop to minimize unintended ORF read-through.

tMt51186 Start: 9,327 End: 10,326. Modified terminator of Medicago truncatula gene.

| | | | |
|---|---|---|---|
| intron4 Start: | 7,097 | End: | 8,696. |
| intron3 Start: | 5,404 | End: | 6,265. |
| intron2 Start: | 4,880 | End: | 5,127. |

iAtBAF60 Start: 3,360 End: 3,768. Intron of Arabidopsis thaliana BAF60 homolog (CHC1 by the Chromatin database) inserted in GUS coding sequence to prevent bacterial expression.

cGtoRG30-01 (SEQ ID NO: 4) Start: 2,734 End: 9,320. A CDS of a soy R-gene that encodes a protein containing toll/interleukin receptor-1 (TIR), nucleotide-binding site (NBS), and leucine rich repeat (LRR) domains. This CDS is from an R-gene in Chromosome 3 of G. tomentella PI_505267.

iMt51186 Start: 2,450 End: 2,700 The first intron of the Medicago truncatula gene.

iMt51186 Start: 2,450 End: 2,574 Truncated version of the first intron of the Medicago truncatula gene.

start Start: 2,313 End: 2,313 Transcription start based on cDNA/gDNA alignment.

prMt51186 Start: 217 End: 2,721 The promoter from the Medicago truncatula gene.

xSTOPS Start: 184 End: 195 6-frame stop to minimize unintended ORF read-through

xTAG Start: 144 End: 183 40 bp site for plant insert intactness testing and to stop readthrough ORFs.

bNRB Start: 101 End: 125 Right Border Repeat

bNRB Start: 4 End: 143 Right border region of T-DNA of Agrobacterium tumefaciens nopaline ti-plasmid.

The above examples clearly illustrate the advantages of various embodiments of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

1. A polypeptide selected from:
(a) a polypeptide comprising the amino acid sequence shown in SEQ ID NO: 5, or any portion thereof, and further comprising a heterologous amino acid sequence attached thereto, wherein expression of the polypeptide or portion thereof in a plant confers increased pathogen resistance on the plant;
(b) an isolated polypeptide having at least 99%, at least 95%, or at least 90% sequence identity with the amino acid sequence of SEQ ID NO: 5, wherein expression of the polypeptide confers increased pathogen resistance on the plant; or
(c) a fusion polypeptide comprising the amino acid sequence of SEQ ID NO: 5, or the polypeptide of (a) or (b).

2. A nucleic acid molecule comprising:
(a) a nucleotide sequence encoding a protein comprising an amino acid sequence having at least 90%, at least 95% or at least 99% sequence identity to SEQ ID NO: 5, wherein said nucleotide sequence comprises a heterologous nucleic acid sequence attached thereto and expression of the nucleic acid molecule in a plant confers increased pathogen resistance on the plant;
(b) an isolated nucleotide sequence encoding a polypeptide having at least 99%, at least 95%, or at least 90% sequence identity with the amino acid sequence of SEQ ID NO: 5, wherein expression of the polypeptide confers increased pathogen resistance on the plant;
(c) the nucleotide sequence of (a) or (b) comprising a sequence of any one of SEQ ID NOS: 2-4 and 11-12; or
(d) the nucleotide sequence of (a) or (b) having at least 99%, at least 95%, or at least 90% sequence identity to any one of SEQ ID NOs: 2-4 and 11-12.

3. An expression cassette comprising the nucleic acid molecule of claim 2.

4. The expression cassette of claim 3, wherein the nucleic acid molecule is operably linked to a promoter capable of directing expression in a plant cell.

5. The expression cassette of claim 4, wherein the promoter is an endogenous promoter.

6. The expression cassette of claim 4, wherein the promoter is an exogenous promoter.

7. The expression cassette of claim 4, wherein the promoter comprises any one of SEQ ID NOS: 13-15.

8. A vector comprising the nucleic acid molecule of claim 2.

9. A transgenic cell comprising the nucleic acid molecule of claim 2.

10. A plant having stably incorporated into its genome a nucleic acid sequence operably linked to a promoter active in the plant, wherein the nucleic acid sequence encodes a polypeptide having:
(a) an amino acid sequence comprising at least 90% sequence identity, to SEQ ID NO: 5; or
(b) an amino acid sequence as set forth in SEQ ID NO: 5,
wherein said nucleic acid sequence is heterologous to the plant, and wherein expression said nucleic acid sequence in the plant results in an increased pathogen resistance as compared to a control plant not comprising the nucleic acid sequence.

11. The plant of claim 10, wherein
(a) the nucleic acid sequence comprises at least 90% identity, or at least 95% identity to any one of SEQ ID NOs: 2-4 and 11-12; or
(b) the nucleic acid sequence is SEQ ID NO: 2, 3, 4, 11 or 12.

12. The plant of claim 10, wherein the nucleic acid sequence is introduced into the genome by transgenic expression.

13. The plant of claim 10, wherein the promoter is an endogenous promoter.

14. The plant of claim 13, wherein the endogenous promoter comprises at least 95% sequence identity to SEQ ID NO: 15, or wherein the endogenous promoter comprises SEQ ID NO: 15.

15. The plant of claim 10, wherein the promoter is a heterologous promoter, and wherein the heterologous promoter comprises at least 95% sequence identity to SEQ ID NO: 13 or 14, or where in the heterologous promoter comprises SEQ ID NO: 13 or 14.

16. The plant of claim 10, wherein the promoter is a constitutive promoter, inducible promoter, or a tissue-specific promoter

17. The plant of claim 10, wherein the plant is a dicot plant.

18. The plant of claim 17, wherein the dicot plant is a soybean plant or an elite soybean plant.

19-20. (canceled)

21. The plant of claim 17, wherein the plant comprises:
(a) an agronomically elite plant having a commercially significant yield and/or commercially susceptible vigor, seed set, standability, threshability, abiotic/biotic resistance, or herbicide tolerance; or
wherein the plant has increased resistance to any one of the following pathogens: soy cyst nematode, bacterial pustule, root knot nematode, frog eye leaf spot, phytophthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme.

22. (canceled)

23. The plant of claim 17, wherein the plant is a soybean plant and wherein the soybean plant has increased resistance to ASR as compared to the control plant.

24-32. (canceled)

33. An elite Glycine max plant comprising in its genome a nucleic acid sequence from a donor Glycine plant, wherein the donor Glycine plant is a different strain from the elite Glycine max plant, and wherein the nucleic acid sequence encodes at least one polypeptide having at least 90% identity or 95% identity to SEQ ID NO: 5, wherein said polypeptide confers increased pathogen resistance on the elite Glycine max plant as compared to a control plant not comprising said nucleic acid sequence.

34. The plant of claim 33, wherein the donor Glycine plant is a Glycine tomentella plant, or a progeny thereof.

35. The plant of claim 34, wherein the Glycine tomentella plant is a plant of Glycine tomentella accession line PI505267 or a progeny thereof.

36. The plant of claim 33, wherein the nucleic acid sequence has at least 90% identity, at least 95% identity, or, 100% identity to any one of SEQ ID NOs: 2-4 and 11-12.

37-52. (canceled)

53. A method for producing a transgenic plant with improved resistance against ASR, comprising: introducing the nucleic acid molecule of claim 2 into a recipient plant to obtain a transgenic plant, wherein the transgenic plant has increased resistance against ASR compared to the recipient plant.

54. A method of producing a soybean plant having increased pathogen resistance, the method comprising the steps of:
a) providing a donor soybean plant comprising in its genome heterologous nucleic acid sequence encoding at least one polypeptide having at least 90% identity, at least 95% or 100% identity to SEQ ID NO: 5, wherein expression of said nucleic acid sequence confers to said donor soybean plant increased pathogen resistance as compared to another donor soybean plant not comprising said nucleic acid sequence in its genome;
b) crossing the donor soybean plant of a) with a recipient soybean plant not comprising said nucleic acid sequence; and
c) selecting a progeny plant from the cross of b) by detecting the presence of the nucleic acid sequence, or selecting a plant with increased pathogen resistance, thereby producing a soybean plant having increased pathogen resistance.

55-68. (canceled)

69. A method of conferring increased ASR resistance to a plant, comprising:
a) introducing into the genome of the plant, a nucleic acid molecule operably linked to a promoter active in the plant, wherein the nucleic acid sequence is stably incorporated into the genome, wherein the nucleic acid sequence encodes a polypeptide having
(i) an amino acid sequence comprising at least 90%, or at least 95% identity to SEQ ID NO: 5, or
(ii) an amino acid sequence as set forth in SEQ ID NO: 5,
wherein said nucleic acid sequence is heterologous to the plant, and
wherein expression of said nucleic acid sequence in the plant increases ASR resistance of the plant compared to a control plant not expressing said nucleic acid sequence.

70. The method of claim 69, wherein the nucleic acid sequence is introduced into the genome of the plant by transformation.

71. The method of claim 69, wherein the nucleic acid sequence is introduced into the genome of the plant by crossing a donor plant comprising the nucleic acid sequence with the plant to produce a progeny plant having increased ASR resistance.

72-73. (canceled)

74. The method of any of claim 69, wherein the promoter is an endogenous promoter, and wherein optionally the endogenous promoter comprises SEQ ID NO: 15.

75. (canceled)

76. The method of any one of claim 69, wherein the plant is a dicot plant, and wherein the dicot plant is a soybean plant.

77. (canceled)

78. A plant produced by the method of claim 69.

79-81. (canceled)
